SEQUENCE LISTING (4 sequences)

SEQ ID NO: 1 - protein, 25 aa, Homo sapiens
CVVRFQYYEDSSGKSILYVVEEPEC

SEQ ID NO: 2 - protein, 20 aa, Homo sapiens
KKFDREPYMTENTCNRYCRD

SEQ ID NO: 3 - protein, 5 aa, Homo sapiens
CLNNE

SEQ ID NO: 4 - protein, 7 aa, Homo sapiens
MGLKIGD

(19) United States
(12) Patent Application Publication
(10) Pub. No.: US 2006/0142548 A1
(43) Pub. Date: Jun. 29, 2006

(54) Methods of identifying and designing cell surface receptor inhibitors

(76) Inventors: M. Amin Arnaout, Chestnut Hill, MA (US); Thilo Stehle, Somerville, MA (US); Simon Goodman, Griesheim (DE)

Correspondence Address: FISH & RICHARDSON PC, P.O. BOX 1022, MINNEAPOLIS, MN, 55440-1022, US

(21) Appl. No.: 11/042,964

(22) Filed: Jan. 24, 2005

Related U.S. Application Data
(63) Continuation of application No. 10/769,497, filed on Jan. 30, 2004, now abandoned.
(60) Provisional application No. 60/444,609, filed on Jan. 30, 2003.

(51) Int. Cl. G06F 19/00; C07K 14/705

(52) U.S. Cl. 530/350; 702/19

(57) ABSTRACT

The methods described herein provide for ways of modulating, specifically inhibiting, integrin activation. The methods include identifying compounds such as naturally occurring and non-naturally occurring polypeptides and small molecules which bind to the βA domain of an integrin thus mimicking the contact between the CD loop with βA domain. Contacting the βA domain in this manner locks the integrin such that it is unable to be activated.

### CROSS REFERENCE TO RELATED APPLICATIONS

This application is a continuation (and claims the benefit of priority under 35 USC 120) of U.S. application Ser. No. 10/769,497 filed Jan. 30, 2004, which claims priority to U.S. application Ser. No. 60/444,609 filed Jan. 30, 2003. The disclosure of the prior applications are considered part of (and is incorporated by reference in) the disclosure of this application.

### TECHNICAL FIELD

This invention relates to methods of identifying and designing cell surface receptor inhibitors, particularly integrin inhibitors.

### STATEMENT REGARDING GOVERNMENT SPONSORED RESEARCH

This research was supported by grant numbers DK48549 and HL70219 awarded by the National Institutes of Health (NIDDK and NHLBI).

### BACKGROUND

Integrins are adhesion receptors that mediate vital bi-directional signals during morphogenesis, tissue remodeling and repair (reviewed in 2). Integrins are heterodimers formed by noncovalent association of an α and a β subunit, both type I membrane proteins with large extracellular segments. In mammals, eighteen α and eight β subunits assemble into 24 different receptors. Integrins depend on divalent-cations to bind their extracellular ligands. Although these ligands are structurally diverse, they all employ an acidic residue during integrin recognition. Specificity for a particular ligand is then determined by additional contacts with the integrin. High affinity binding of integrins to ligands is usually not constitutive, but is elicited in response to cell “activation” signals (so-called “inside-out” signaling) that alter the tertiary and quaternary structure of the extracellular region, making the integrin ligand-competent. Ligand binding in turn induces structural rearrangements in integrins that trigger “outside-in” signaling (reviewed in 4).

Integrins can be grouped into two classes based on the presence or absence of α˜180 amino acid A-type domain (αA or I domain; see 18). In the nine αA-containing integrins (αA-integrins), αA is the major ligand binding site. Thus, isolated αA binds directly and in a divalent-cation-dependent manner to physiologic ligands with affinity equal to that of the respective ligand-competent heterodimer.¹⁸ The structures of isolated αA domains in “liganded” (high-affinity) and “unliganded” (low-affinity) conformations have revealed how this domain interacts with ligands.^{19,30} A metal ion is coordinated at the ligand-binding interface of αA through a conserved five amino acid motif, the metal-ion-dependent-adhesion-site (MIDAS), and the metal coordination is completed by a glutamate from the ligand or, in its absence, by a water molecule.^{19,20,29} In αA-lacking integrins, we proposed that ligand recognition is supported by an αA-like domain (αA) present in all integrin β subunits.¹⁹ The a subunit is also believed to participate in ligand recognition; we have recently defined its binding site to the prototypical ligand RGD.²³

### SUMMARY

The invention features methods for identifying compounds which bind and modulate integrins. The compounds identified by the invention can inhibit integrins in a manner referred to herein as “deadbolt inhibition.” The region involved in deadbolt inhibition includes the βA domain strand-F/α7 loop contacted by the CD loop of the β tail domain (βTD) of an unliganded integrin (e.g., αVβ3). The contact region between the βA domain and the βTD acts as a regulatable deadbolt to lock the βA domain in an inactive state by preventing strand-F/α7 loop movement associated with the activation-initiated inward movement of the α1 helix. The deadbolt contact is stabilized in place by an additional contact on the same side between the βTD and the hybrid domain and by an ionic bond through an ADMIDAS cation, linking the strand-F/α7 loop to the α1 helix of βA. The compounds (e.g., mimetics of this loop, of the ADMIDAS cation, or of the βTD/βA contact) identified by the methods of the invention act to stabilize the CD loop in locking the integrin in an inactivated state.

The invention features methods for evaluating potential of a compound to associate with a molecule or molecular complex comprising a non-ligand binding site of an integrin βA domain, the method comprising: (a) employing computational means to perform a fitting operation between the compound and β tail domain (βTD) contact region on the strand-F/α7 loop of an unliganded integrin (e.g., αVβ3); and (b) analyzing the results of the fitting operation to quantify said association potential. In certain embodiments, the compound mimics the interaction of a peptide, comprising the amino acid sequence C⁶⁶³VVRFQYYE⁶⁷¹D⁶⁷²S⁶⁷³S⁶⁷⁴G⁶⁷⁵KSILYVVEEPEC⁶⁸⁷ or a fragment thereof, or K⁶¹⁸KFDREPYMTENTCNR⁶³³YCRD or a fragment thereof, with the strand-F/α7 loop of a βA domain of the integrin.

In another aspect, the invention features a method for identifying a candidate selective modulator of the activity of an integrin, the method comprising: (a) modeling test compounds that fit spatially and preferentially into a βA domain non-ligand binding site of an integrin of interest using an atomic structural model of the integrin βA domain, wherein the atomic structural model is generated using amino acid sequence comprising C⁶⁶³VVRFQYYE⁶⁷¹D⁶⁷²S⁶⁷³S⁶⁷⁴G⁶⁷⁵KSILYVVEEPEC⁶⁸⁷ or a fragment thereof, or K⁶¹⁸KFDREPYMTENTCNR⁶³³YCRD or fragment thereof; (b) screening test compounds in a biological assay for integrin activation characterized by binding of a test compound to the βA domain non-ligand binding site of the integrin; and (c) identifying a test compound that selectively modulates the activity of the integrin, and optionally, (d) screening an identified test compound in a biological assay for its ability to prevent interaction of the βA domain and the βTD domain by binding of the identified test compound to the βA domain non-ligand binding site of the integrin, and (e) identifying the screened test compound as a compound capable of selectively modulating the activity of an integrin from the pool of test compounds. In various embodiments: the modulating comprises inhibiting the integrin activation and the modulating comprises inhibiting ligand binding to the integrin.

In another aspect, the invention features a method of identifying a candidate inhibitor of the activity of an integrin, the method comprising: (a) introducing into a suitable computer program information defining a non-ligand binding site of an integrin βA domain, the information comprising a conformation defined by the coordinate atoms as in Table 1 and Table 2, wherein the program displays the three-dimensional structure thereof; (b) creating a three dimensional structure of a test compound in the computer program; (c) superimposing the model of the test compound on the model of the non-ligand binding site of the integrin βA domain; and (c) assessing whether the test compound model fits spatially into the non-ligand binding site. In one embodiment, the atoms include those in Table 3 and Table 4.

In another embodiment, the invention features a method for identifying a candidate integrin modulating compound, the method comprising: (a) generating a three-dimensional structure of the βTD contact region of strand-F/α7 loop of an βA domain of a non-ligand bound integrin; (b) employing the three dimensional structure to design or select the candidate integrin modulating compound, and (c) identifying said candidate integrin modulating compound by the data obtained by steps (a) and (b). In one embodiment, the method also includes: (d) synthesizing the integrin modulating compound; and (e) determining the ability of the integrin inhibitor to bind to the integrin by contacting the modulating compound with the integrin.

In another aspect, the invention features a method for identifying a candidate integrin modulating compound, the method comprising (a) expressing recombinant integrin fragments containing βA domain and βTD domains and (b) employing the protein-protein interaction of these two domains to in screening assays to identify modulators of the βA domain and βTD domain interaction. The method can further include: (c) synthesizing the integrin modulating compound; (d) determining the ability of the integrin modulator to bind to the integrin by measuring the interaction of the modulating compound with the integrin; and (e) using the modulating compound as a basis for drug design.

The invention also features a method of inhibiting activation of an integrin the method comprising contacting a compound to with an integrin thereby locking the βA domain structure of the integrin into a non-activatable form. In various embodiments, the compound mimics an intrachain ligand in its interaction with the integrin, the intrachain ligand comprises the sequence of SEQ ID No:1, or a fragment thereof, or SEQ ID No. 2, or a fragment thereof; and the intrachain ligand is a member of the statin family.

In another aspect, the invention features a method of identifying an integrin modulator comprising: (a) selecting a potential inhibitor by performing rational drug design with the three-dimensional structural coordinates of Table 1 and Table 2, wherein selecting is performed in conjunction with computer modeling; (b) contacting the potential inhibitor with an integrin domain; and (c) detecting the ability of the potential inhibitor for inhibiting the integrin. In various embodiments, detecting the ability of the potential inhibitor for inhibiting the integrin in step (c) is performed using a ligand binding assay; detecting the ability of the potential inhibitor for inhibiting the integrin in step (c) is performed using a cellular-based assay; and the method further includes: (d) growing a supplemental crystal comprising a complex formed between the integrin domain and a first potential inhibitor from step (a), the supplemental crystal effectively diffracts X-rays for the atomic coordinates of the complex a resolution of greater than 4.0 Å; (e) determining the three-dimensional structure of the supplemental crystal; (f) selecting a second potential inhibitor by performing rational drug design with the three-dimensional structure determined for the supplemental crystal, wherein selecting is performed in conjunction with computer modeling;

(g) contacting the second potential inhibitor with the integrin domain; and (h) detecting the ability of the second potential inhibitor for inhibiting the integrin.

Also within the invention are compounds identified by the methods of the invention, with the proviso that the compound is not lovastatin, and pharmaceutical compositions comprising such compounds and a pharmaceutically acceptable carrier.

The invention also features a method for modulating, inhibiting or stimulating binding of ligands or associated proteins to integrins by modifying the interaction of integrin beta-A domain (βA) with the beta-tail domain (βTD). In certain embodiments the integrin is selected from the group consisting of: αVβ1, αVβ3, αVβ5, αVβ6, αVβ8, α3β1, α4β1, α5β1, α6β1, α6β4, α7β1, α9β1, α4β7, gp3b3a, α1β1, α2β1, α10β1, α11β1, LFA-1, MAC-1, or α150β95; the interaction of βA with βTD is investigated using either computational or biochemical or biophysical techniques; and either the βA, the βTD or both serve as structures on which the computational or biochemical or biophysical techniques are based.

The invention also features a method for discovering pharmacologically relevant substances including antibodies, small molecules, polypeptides, peptides, and peptide mimetics, which can perturb or stimulate the interaction of integrin beta-A domain (βA) with the beta-tail domain (βTD). In certain embodiments, the pharmacologically relevant substances modulate (e.g., inhibit) the interaction of the native integrin with its ligands or cofactors.

Definitions

As used herein, “integrin” and “integrin receptor” are used interchangeably. “Integrin” or “integrin receptor” refers to any of the many cell surface receptor proteins, also referred to as adhesion receptors which bind to extracellular matrix ligands or other cell adhesion protein ligands thereby mediating cell-cell and cell-matrix adhesion processes. The integrins are encoded by genes belonging to a gene superfamily and are typically composed of heterodimeric transmembrane glycoproteins containing α- and β-subunits. Integrin subfamilies contain a β-subunit combined with different α-subunits to form adhesion protein receptors with different specificities. The integrins are grouped into two classes, those containing the αA domain and those that do not contain the αA domain. Both classes have a βA domain.

“Compounds” refer to a chemical entity that can comprise a peptide or polypeptide, including antibodies, phage display antibodies, and their biologically active fragments, a small molecules, (e.g., chemically synthesized or of natural origin), or synthetic peptides or polypeptides (e.g., non-naturally occurring polypeptides, e.g., peptoids or peptidomimetics). The compound has the ability to bind the integrin at a non-ligand binding site of the βA domain. The “non-ligand binding site of an integrin βA domain” includes the site with which the CD loop of the β tail domain (βTD) of the β subunit that contacts the strand-F/α7 loop in the βA domain of the β subunit of an unliganded integrin. As such, ligand refers to the naturally occurring ligand that binds the integrin so that it can perform its physiological function or functions. This site is distinguishable from the ligand binding site. Compounds can include synthetic and naturally occurring mimetics of the CD loop or fragments of the CD loop of the β tail domain (βTD) that contacts the strand-F/α7 loop in the βA domain of an unliganded integrin, the α1/strand-A loop or fragments of that loop that contact the hybrid domain of an unliganded integrin, and the ADMIDAS coordination site in the βA domain of an unliganded integrin. A “mimetic” has structural similarity or has similar binding properties as the entity it is mimicking. Compounds can comprise or consist of the mimetic. According to the invention, “compounds” preferably refers to chemical entities as described above, selected from the group consisting of
a) naturally occurring polypeptides, preferably naturally occurring polypeptides of less than 160 kDa and especially of less than 100 kDa, but preferably of more than 32 kDa;
b) synthetic polypeptides, preferably synthetic polypeptides of less than 160 kDa and especially of less than 80 kDa, but preferably of more than 32 kDa;
c) small molecules, preferably small molecules as defined below;
d) antibodies, preferably antibodies selected from the group consisting of monoclonal antibodies, polyclonal antibodies, chimeric antibodies, antibody fusions, and the like;
e) antibody fragments, preferably antibody fragments of antibodies as given under d); and
f) non-peptidic organic molecules, preferably having a formula weight above 150 g/mol and preferably less than 1500 g/mol, more preferred above 250 g/mol and preferably less than 800 g/mol.

“Modulation” refers to regulating or changing the activatability or ligand binding of an integrin. For example, a modulator can inhibit or promote the activation of the integrin or it can prevent or promote ligand binding to the integrin.

A “peptidomimetic” refers to a chemical variant of a polypeptide or a peptide in which the side chains of the polypeptide or peptide are substantially maintained in the variant, yet the chemical backbone of the peptidomimetic is altered relative to the polypeptide or peptide in at least one peptide bond.

A “peptoid” is an oligomer of N-substituted glycines. A peptoid can be synthesized from a variety of different N-alkylglycines that have side chains similar to amino acid side chains, (e.g., as described in Simon et al., (1992) PNAS 89:9367-9371). It can serve as a motif for the generation of chemically diverse libraries of novel molecules. As an alternative to natural polymers, it is a modular system that allows one to synthesize monomers in large amounts. The monomers have a wide variety of functional groups presented as side chains off of an oligomeric backbone, the linking chemistry is high yielding and amenable to automation. The linkage in a peptoid is resistant to hydrolytic enzymes such as proteases. Another advantage is that the monomers are achiral.

A “small molecule” is a molecule of less than 32 kDa, e.g., 0.5 kDa, 1 kDa, 5 kDa, 10, kDa, 15 kDa, 20 kDa, 25 kDa, 30 kDa, or 32 kDa.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is the crystal structure of an extracellular active form of αVβ3 showing 12 domains: four in the α subunit and eight in the β subunit. FIG. 1B is the structure of a Vb3 computationally straightened at the genu (by a 135° flexion and a 120° rotation), resembling the more familiar jellyfish-like earlier EM images of integrins.

FIG. 2 is the structure of extracellular αVβ3 bound to a prototypical Arg-Gly-Asp (RGD) ligand determined after allowing the cyclic RGDf[N-Me]V to diffuse into existing αVβ3 crystals grown in the presence of Mn²⁺²³. The RGD sequence occupies a shallow crevice between the propeller and βA domains (FIG. 2A), with the Arg and Asp residues exclusively contacting the propeller and βA domains respectively. FIG. 2B is a ball and stick representation of tertiary and quaternary changes observed in the liganded structure. The tertiary changes are largely confined to βA and allow the ligand Asp to make direct contact with βA through a second metal ion. FIG. 2C shows schematized small quaternary changes, observed when the unliganded and liganded structures are superimposed at their αβ leg sections (calf-½ and βTD domains): the propeller undergoes a small rotation at the propeller/thigh interface with the βA and hybrid domains following in concert. In addition, the propeller and βA domains move closer together at the peptide-binding site. In the “closed” structure, a water molecule completes coordination of the metal ion (FIG. 2D) and the threonine of loop 2 and aspartate of loop 3 form respectively indirect and direct bonds to the metal ion. In the open conformation (FIG. 2E), the metal ion moves by ˜2 Å, resulting in a subtle change in its coordination: the threonine now contacts it directly, whereas the aspartate contact is via a water molecule. A ligand Glu (indicated in gold in FIG. 2E) replaces the water molecule at the sixth metal coordination site^{19,20,29}; this face of αA has consequently been named the metal ion-dependent adhesion site (MIDAS)¹⁹.

FIG. 3A is a schematic showing that the changes in metal ion position and coordination are linked to a 2 Å movement of loop 1 towards loop 2. FIG. 3B is a schematic showing large tertiary changes in two switch regions linked to tertiary changes in metal coordination in the closed and open forms of αA are strikingly similar respectively to those found in the inactive and active states of G-proteins. When βA is liganded, it displays significant tertiary changes that reshape the loops surrounding its ligand-binding site. These changes resemble in magnitude and direction those seen in the transition from closed to open αA and are likewise triggered by a similar movement of the α1 helix (compare FIGS. 3A and 3C). FIG. 3D is a schematic showing that height and position of the C-terminal α7 helix relative to the central β-sheet in unliganded βA more closely matches that of open αA when these structures are superimposed using TOP.

FIG. 4 shows a schematic of the structure of the α5β1-FN complex modeled on that of liganded αVβ3, aided by the observation that the crystal structures of RGD in the αVβ3-bound peptide²³ and in FN⁶¹ are almost identical.

FIG. 5 is a schematic showing that the flexible 14-17-residue C-terminal linker (C-linker) connecting αA to the propeller contains an invariant glutamic acid (Glu320 in CD11b) within a conserved motif that just follows the α7 helix of αA and as a result of the 10 Å downward movement of this helix, this invariant glutamic acid is likely to contact the MIDAS cation in βA directly as a ligand-mimic, forming the core of an interface between αA and βA that locks αA in the open state⁶⁷.

FIG. 6A is a schematic showing integrin activation can likewise be triggered from either its head or its tails, the latter associated with the separation of the feet and legs. FIG. 6B is a schematic showing that the flexible CD loop of the βTD contacts strand-F/α7 loop (Ser674 of the former contacts Val332 in βA) in unliganded αVβ3, a region in βA that undergoes the most dramatic change upon RGD binding (as seen in FIG. 3C).

FIG. 7A is a schematic showing the α1-strand/A loop of the βTD contacting D/D′ (C³⁷⁴LNNE; SEQ ID NO:3) and D″/E (M³⁸⁷GLKIGD; SEQ ID NO:4) loops of the hybrid domain (Table 2) in the unliganded structure. FIG. 7B shows coordination of the ADMIDAS (adjacent to metal-ion-dependent-adhesion-site) cation in the unliganded structure.

FIG. 8 is a graph showing ligand binding by the αMβ2 integrin. The x-axis shows increasing calcium concentration in the presence of 1 mM Mg²⁺ and the y-axis shows the percent of normal of the iC3b test ligand binding. Binding of the mutant membrane-bound integrin (D¹⁴¹D¹⁴²/A¹⁴¹A¹⁴²) to physiologic ligand increases by 1.5 to 2-fold. Similar results were obtained for an analogous mutation in αVβ3.

FIG. 9 is a table of the atomic coordinates of αVβ3 amino acids.

FIG. 10 is a table of the atomic coordinates of αVβ3 amino acids.

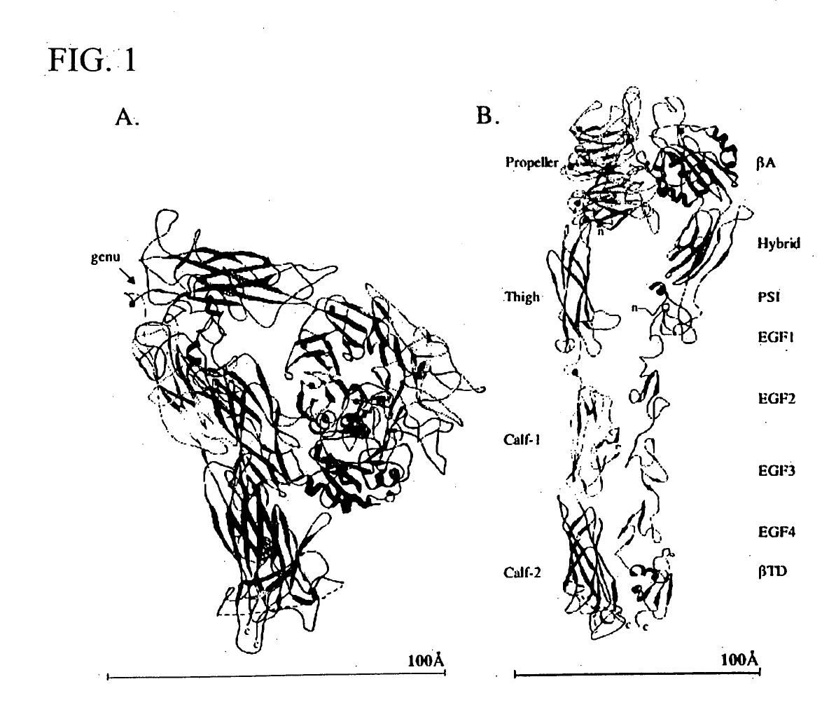

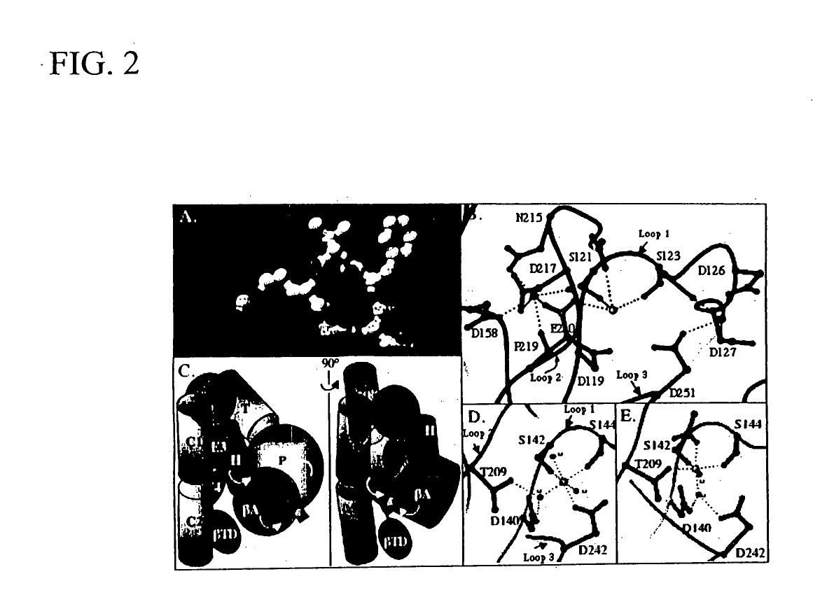

### DETAILED DESCRIPTION

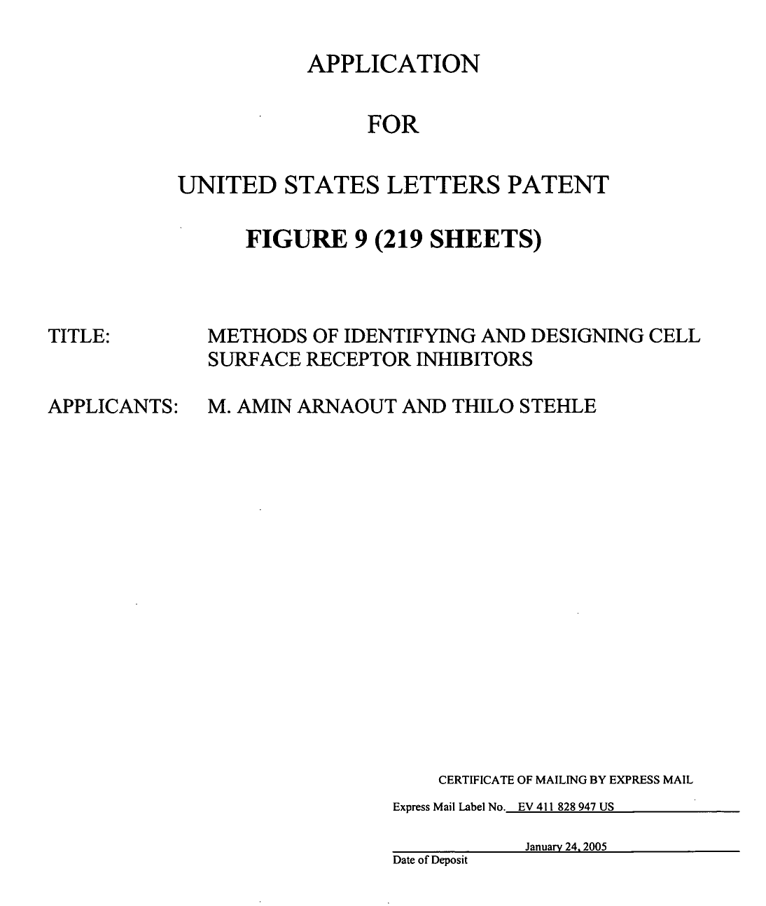

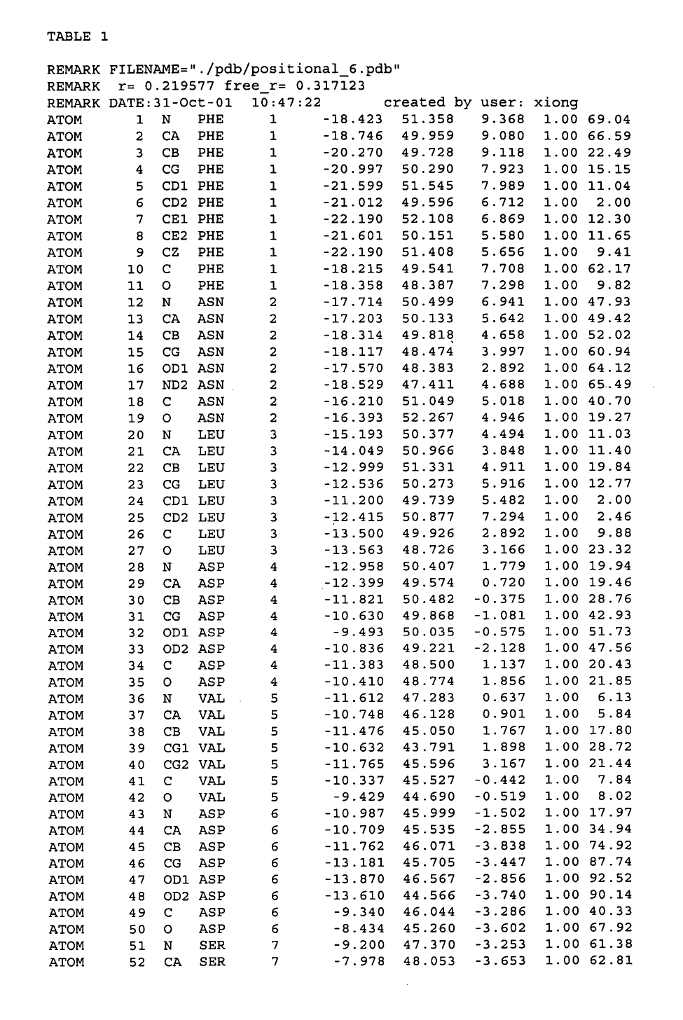

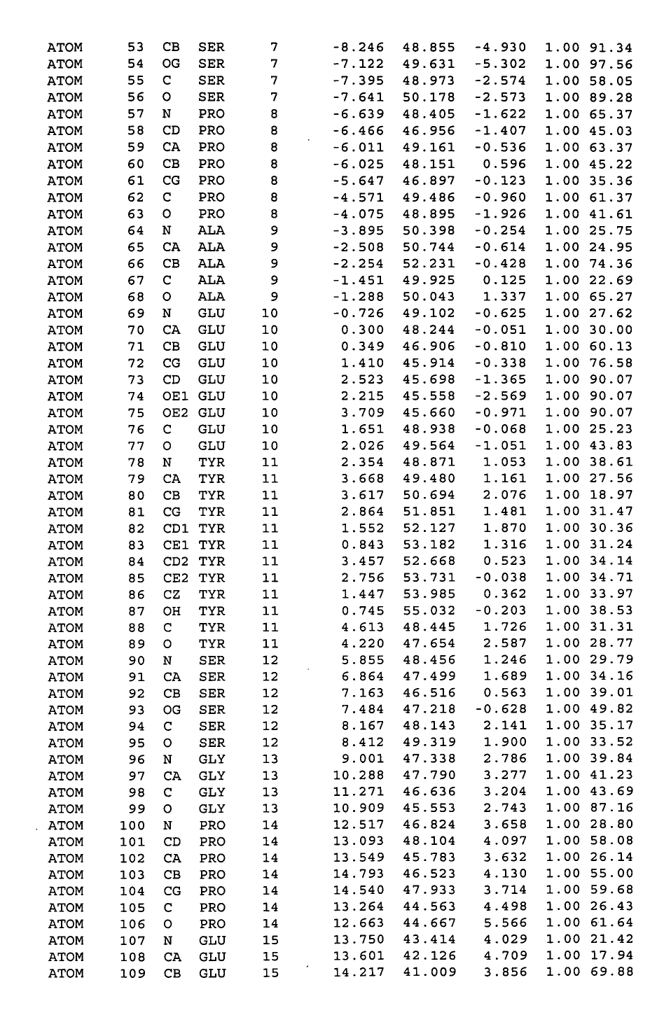

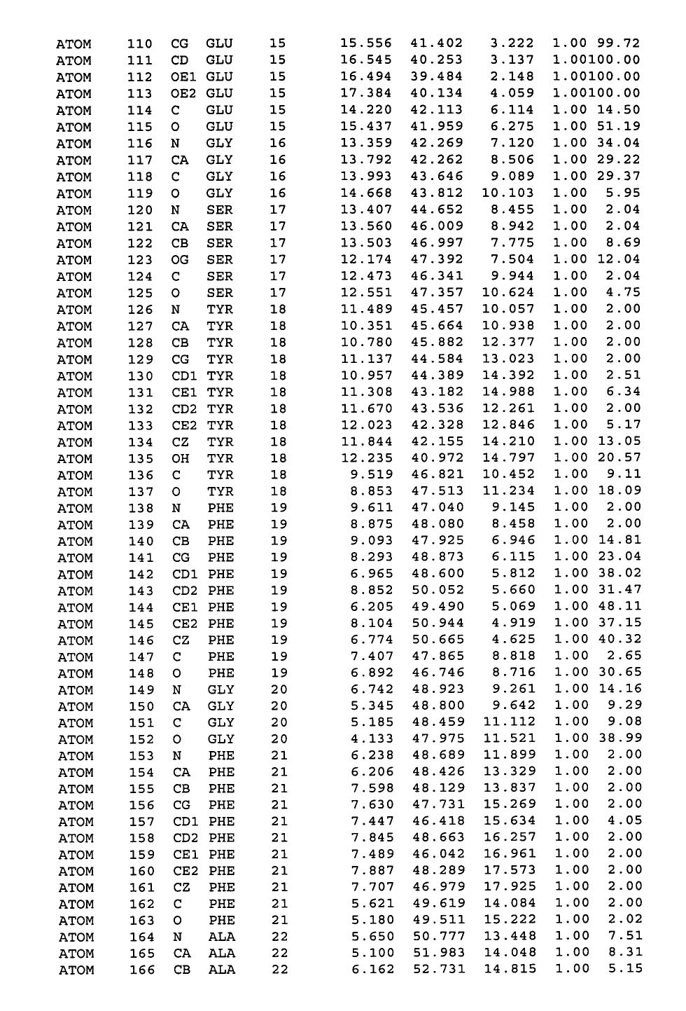

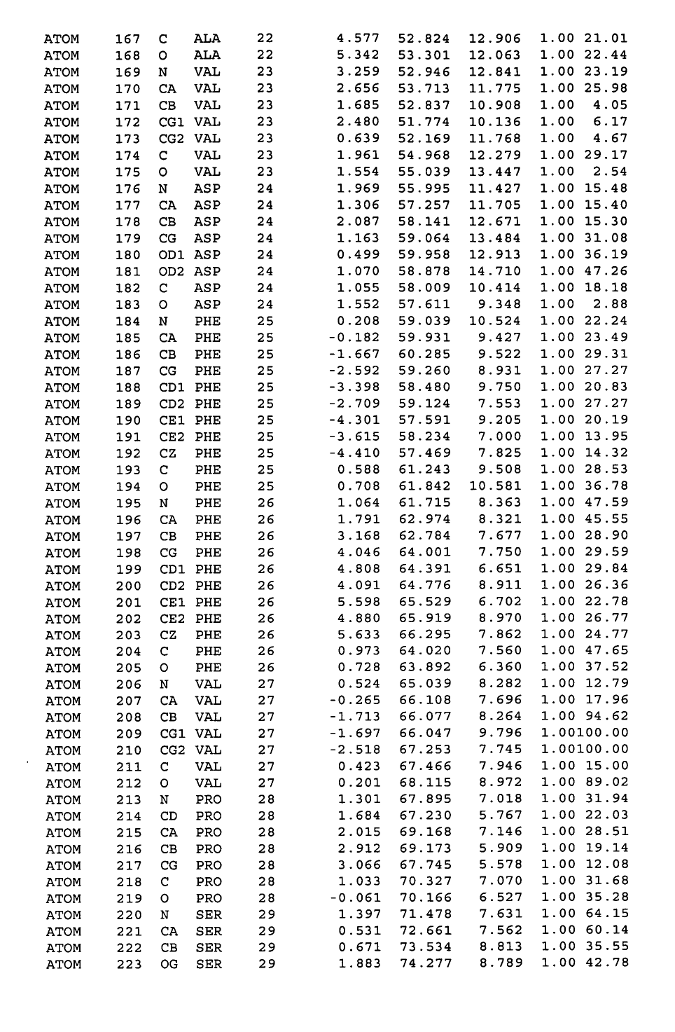

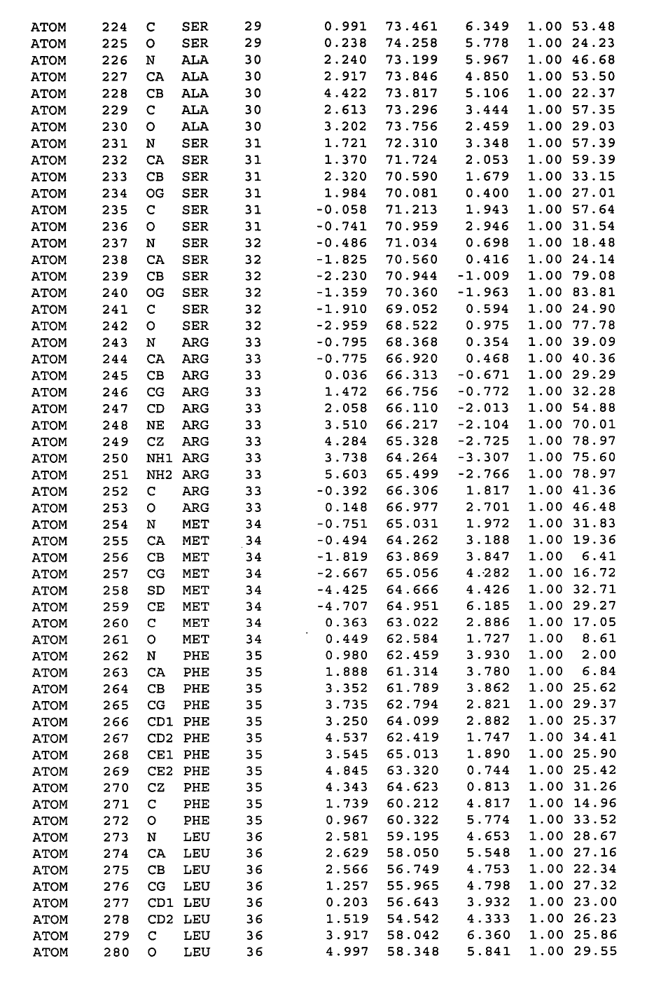

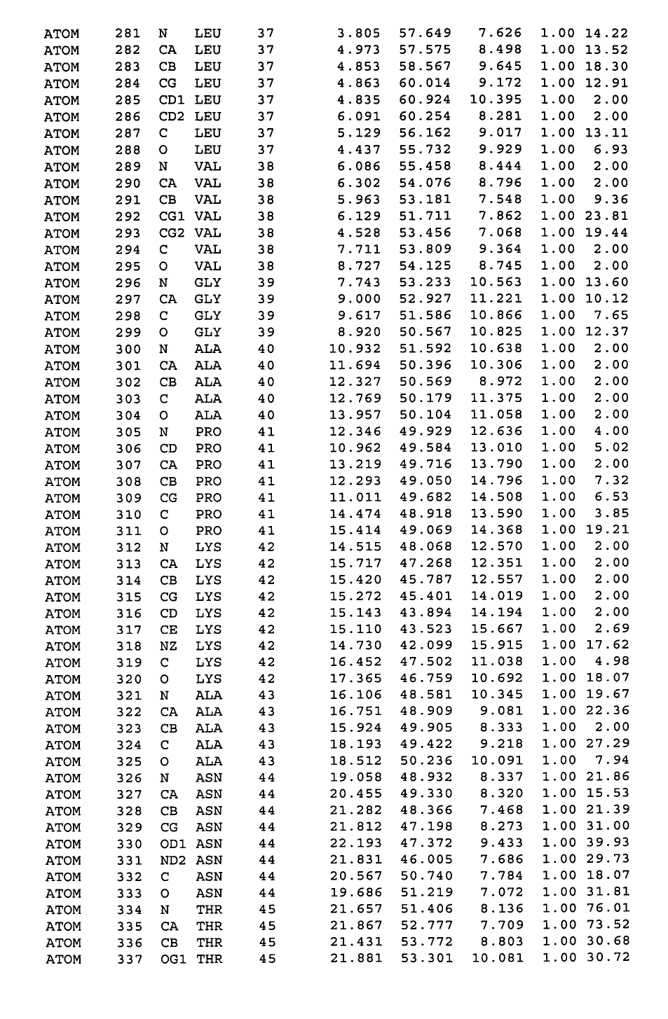

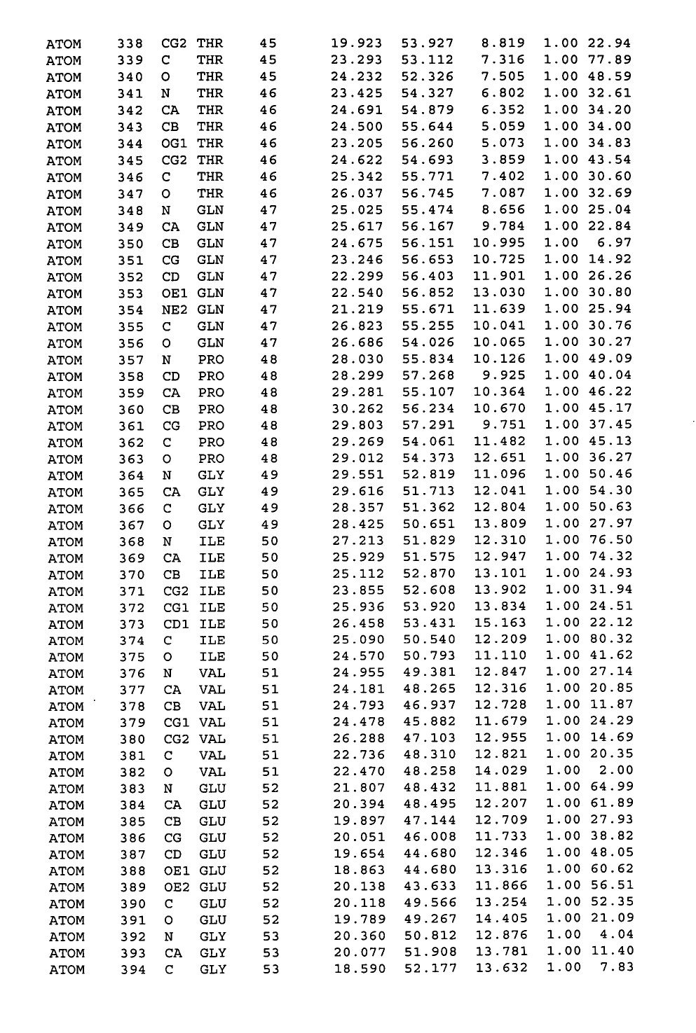

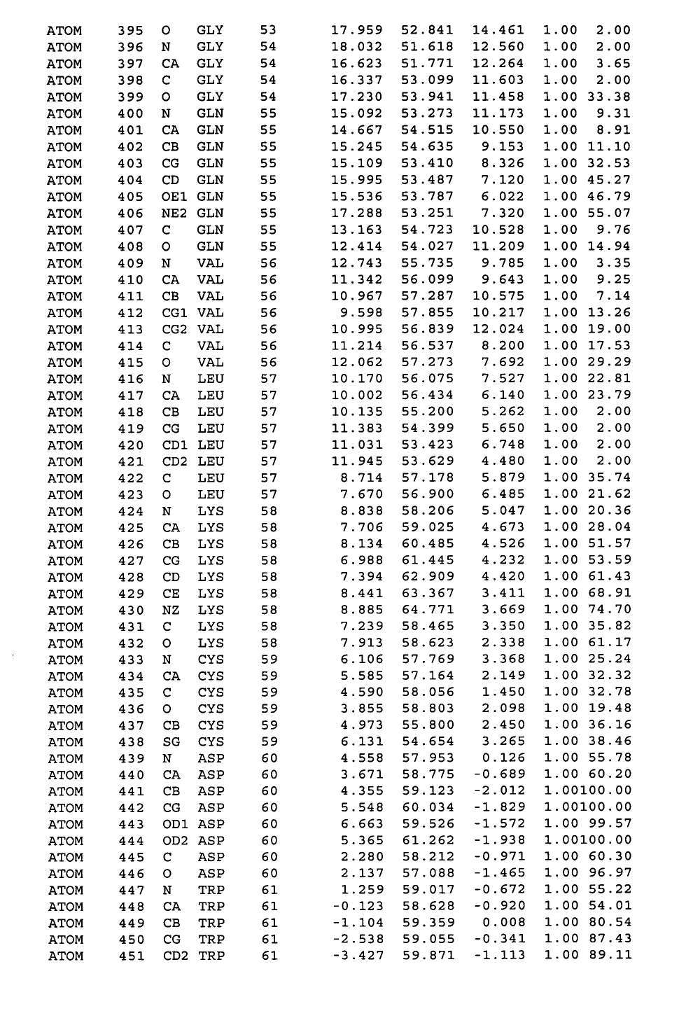

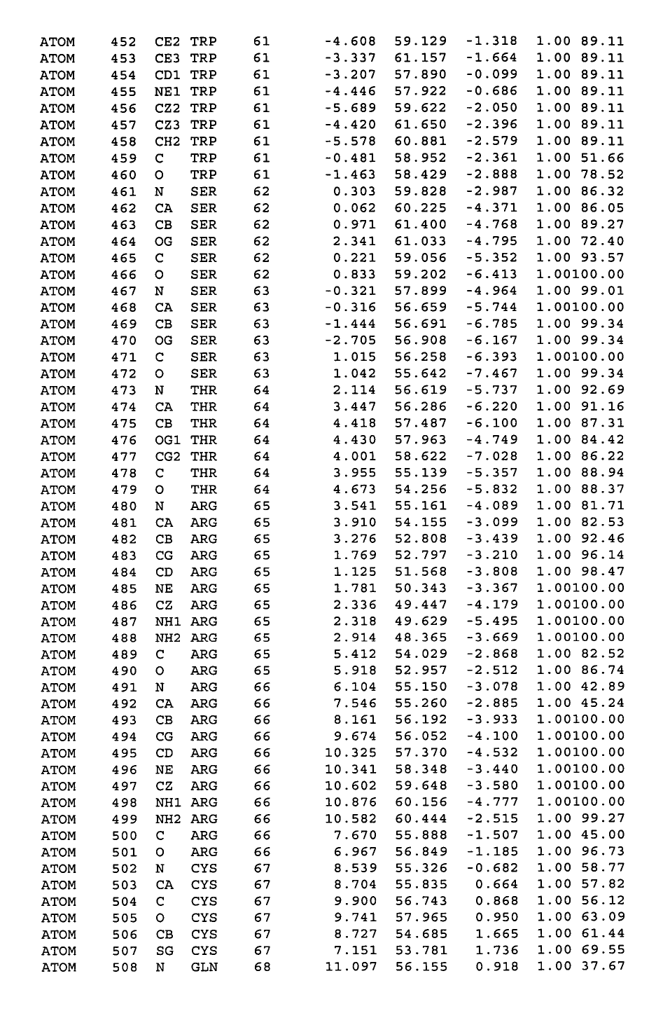

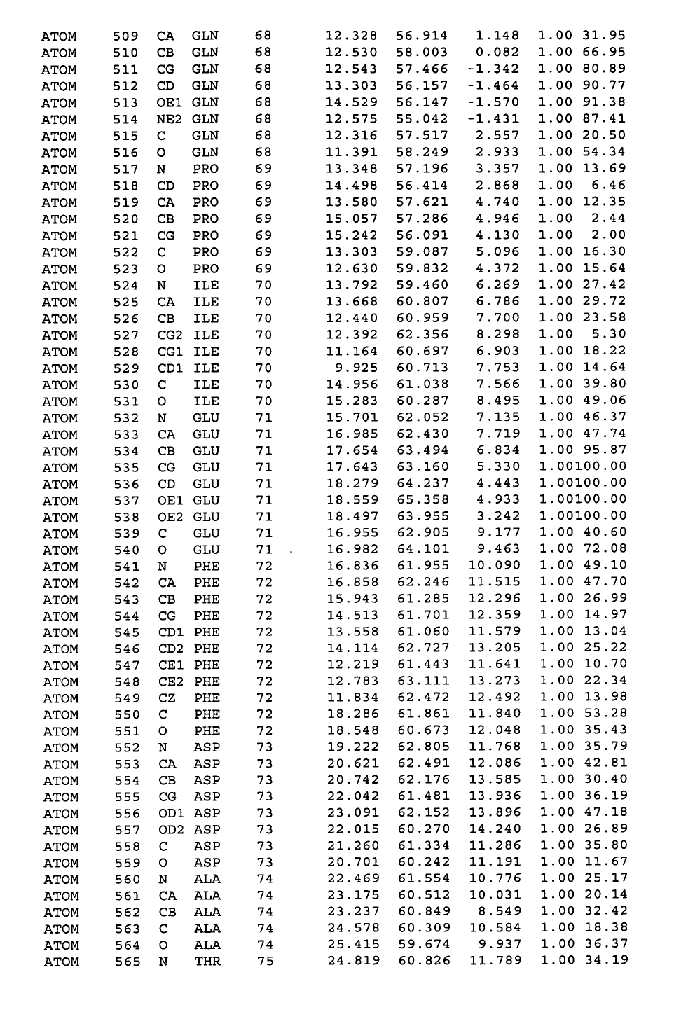

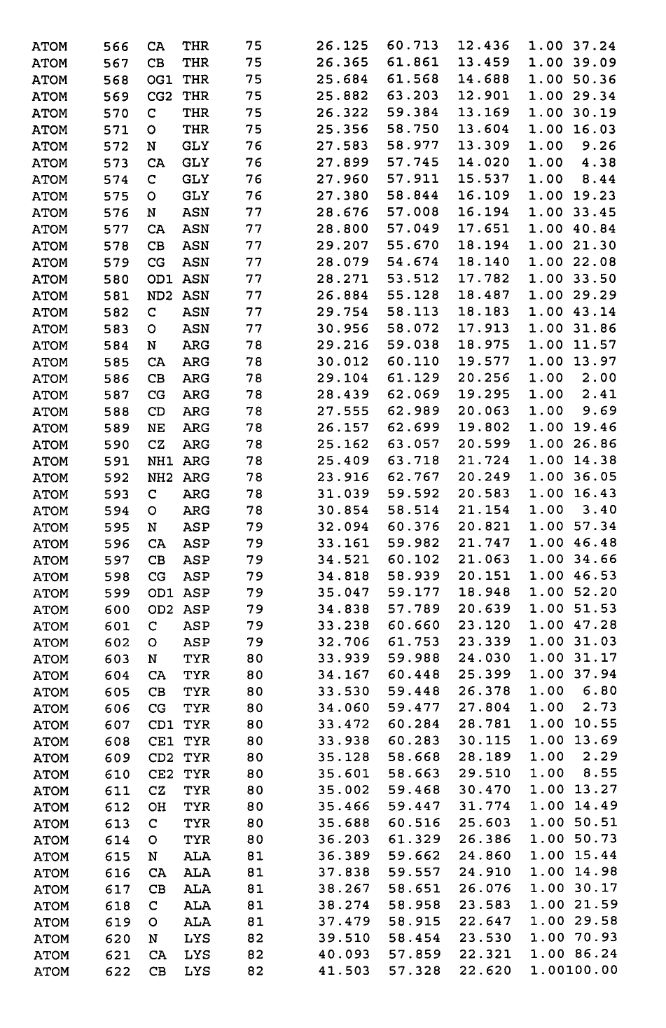

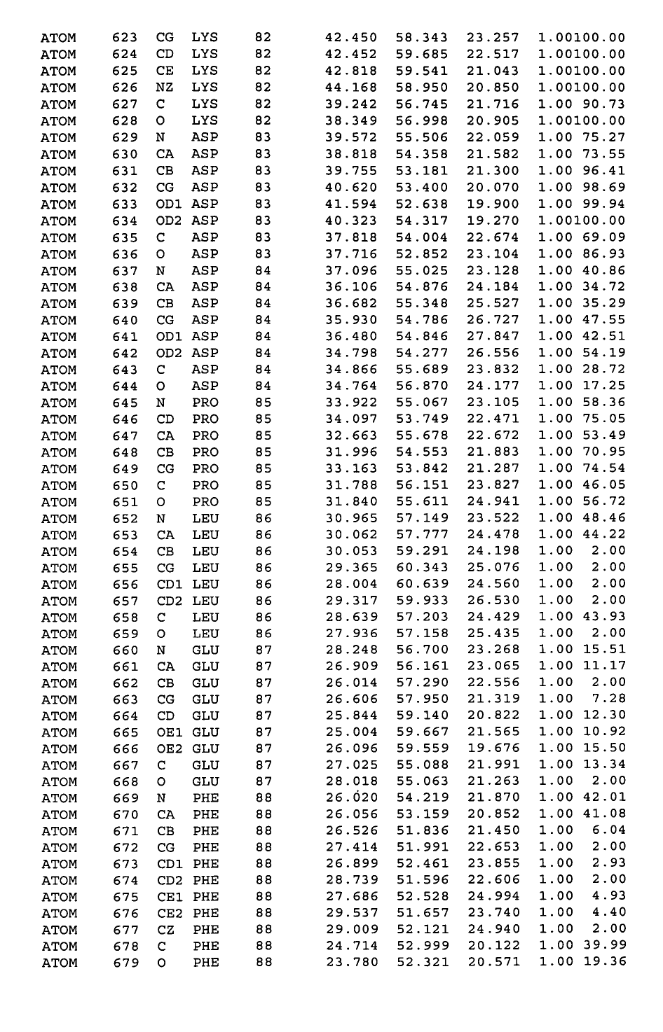

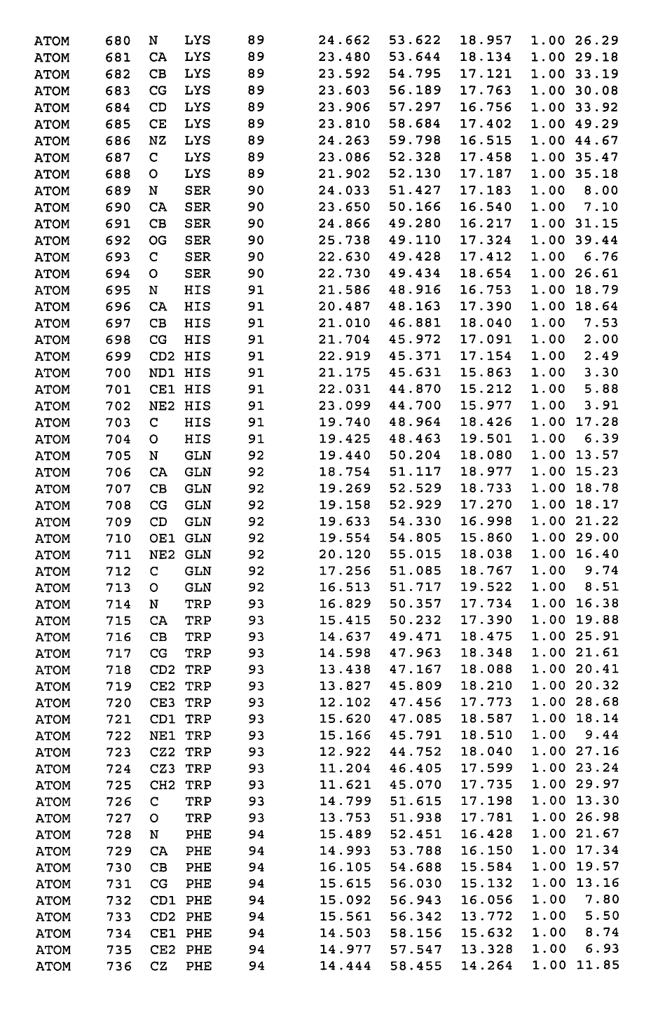

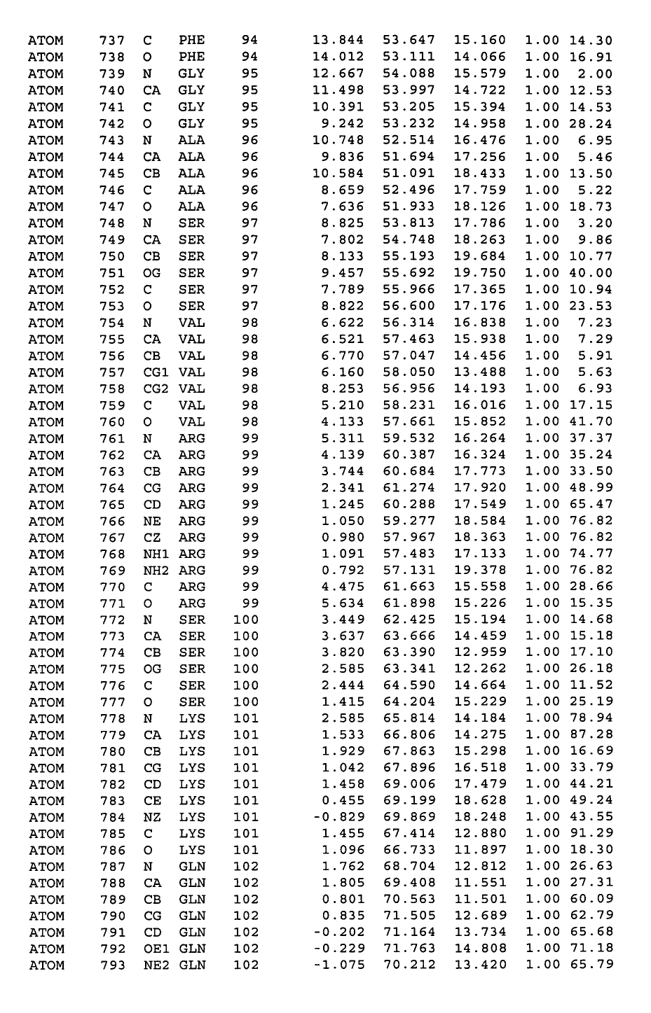

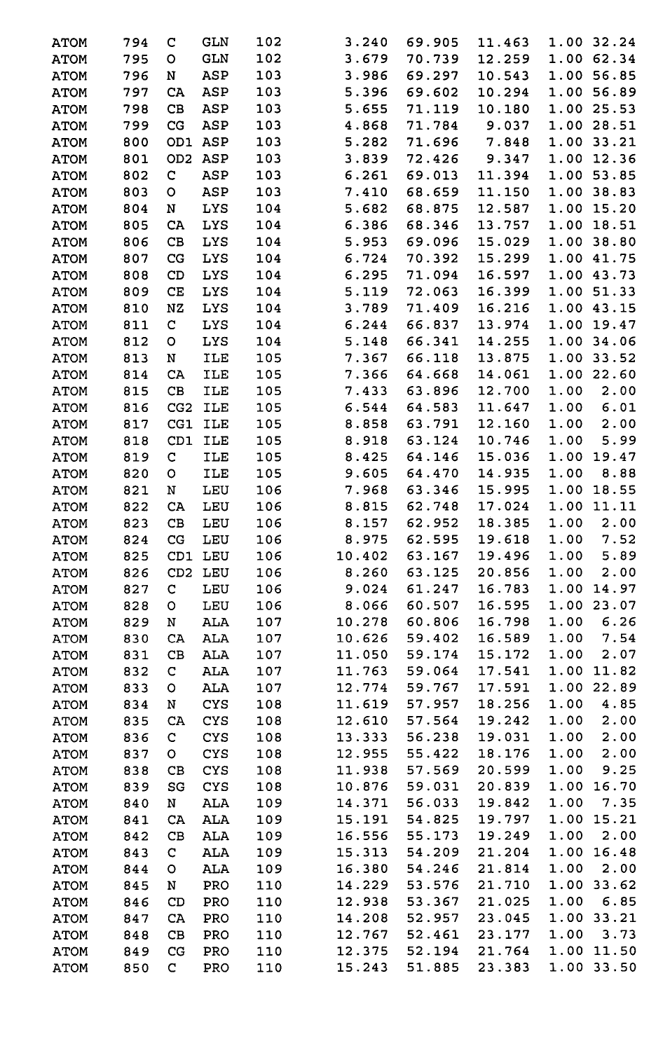

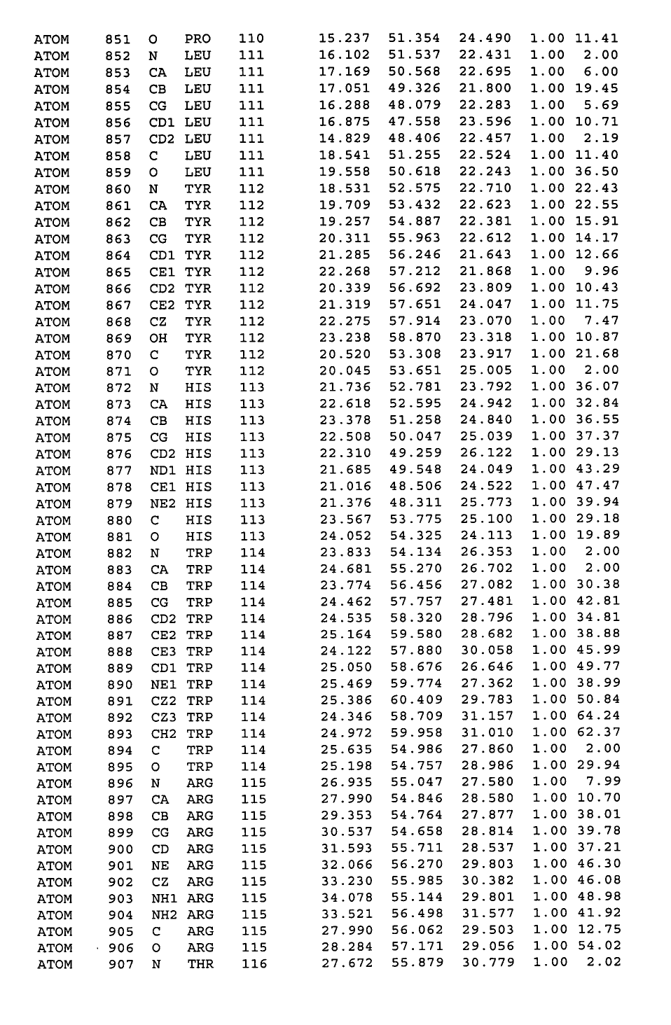

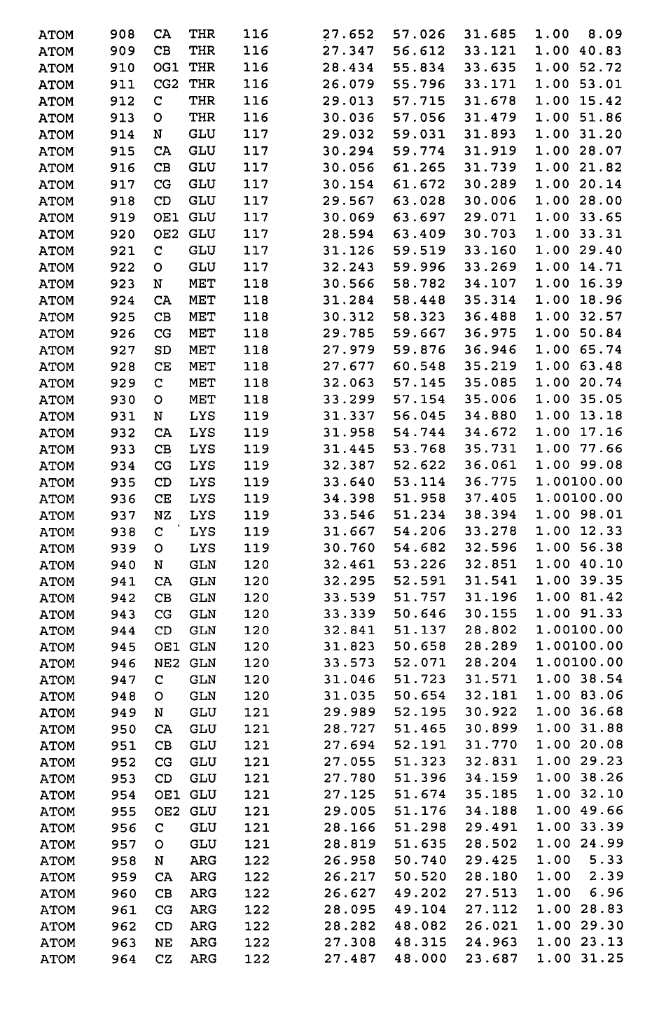

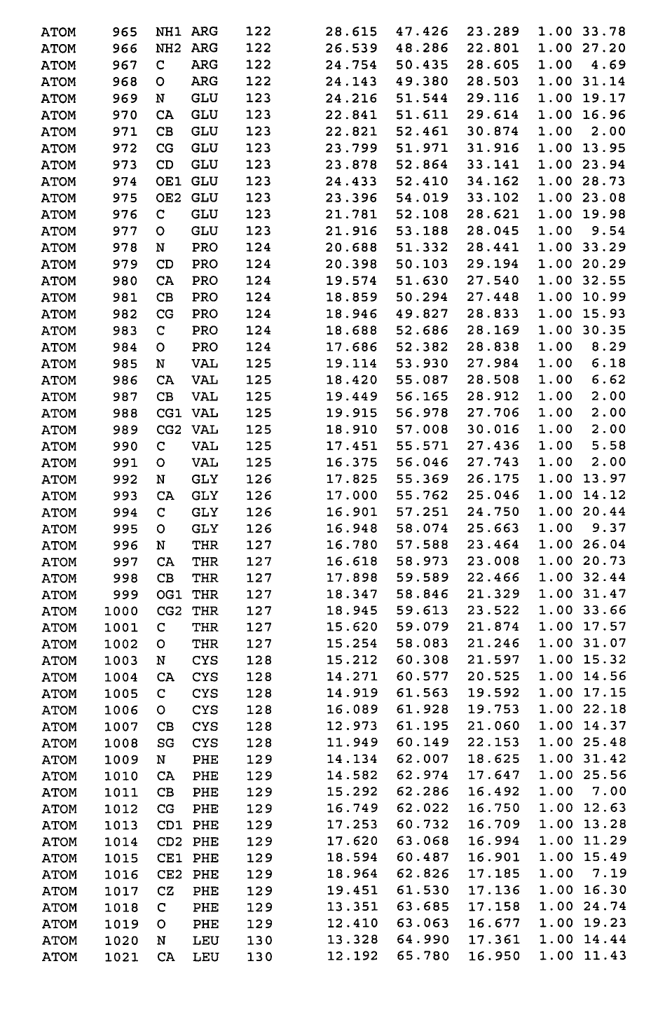

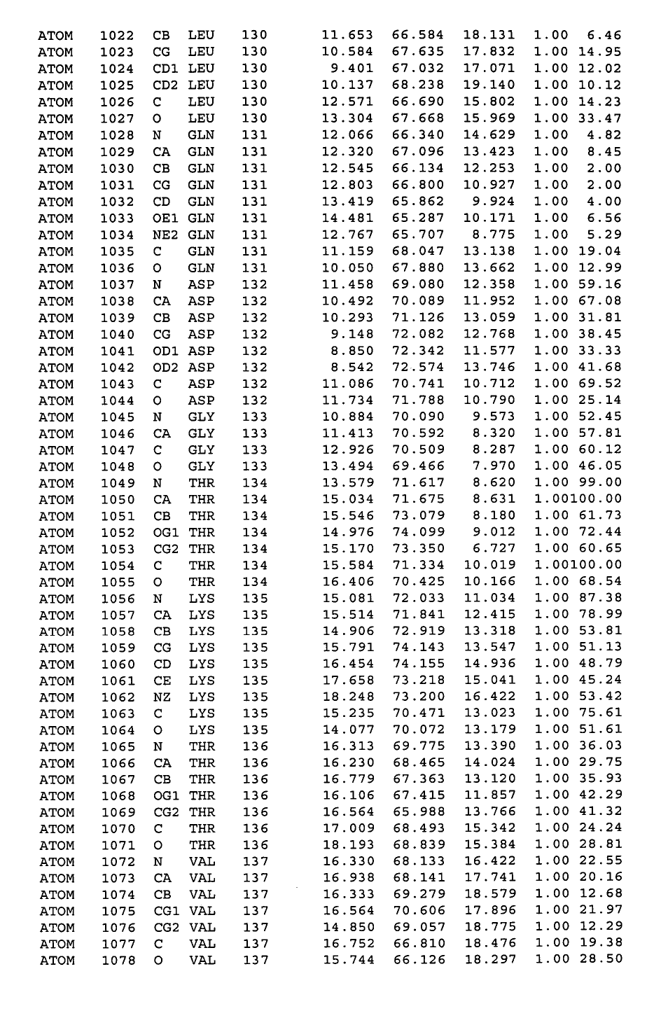

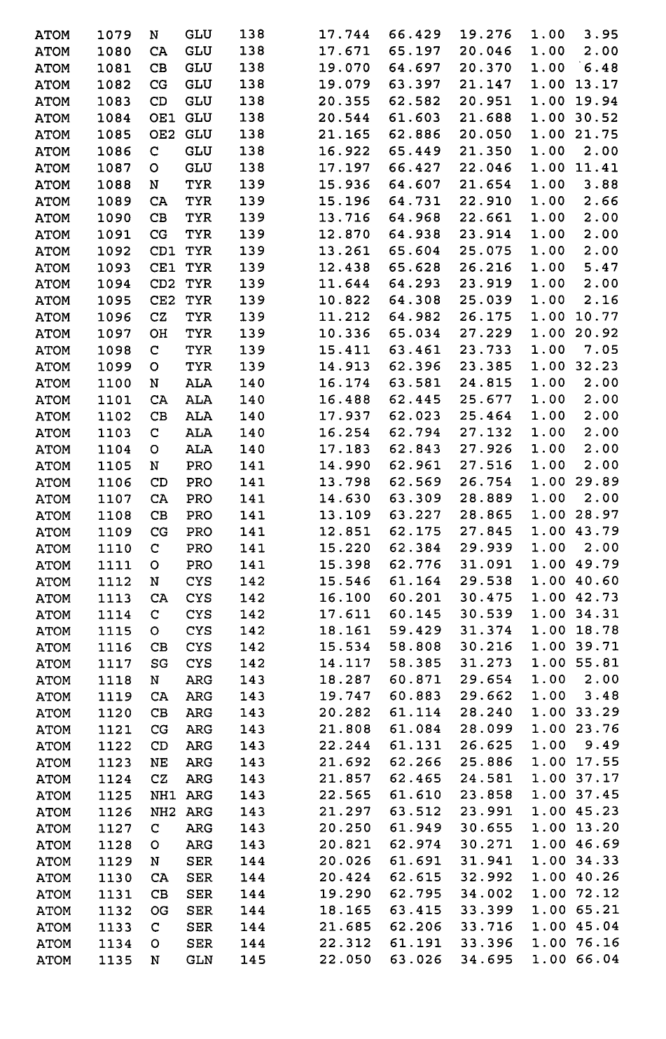

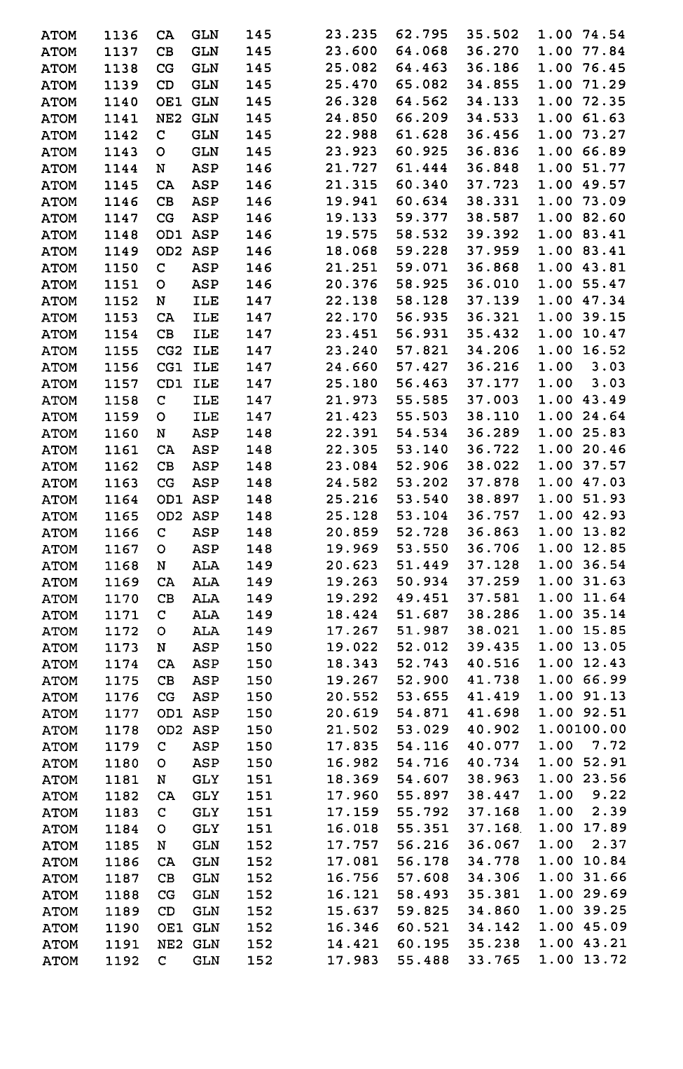

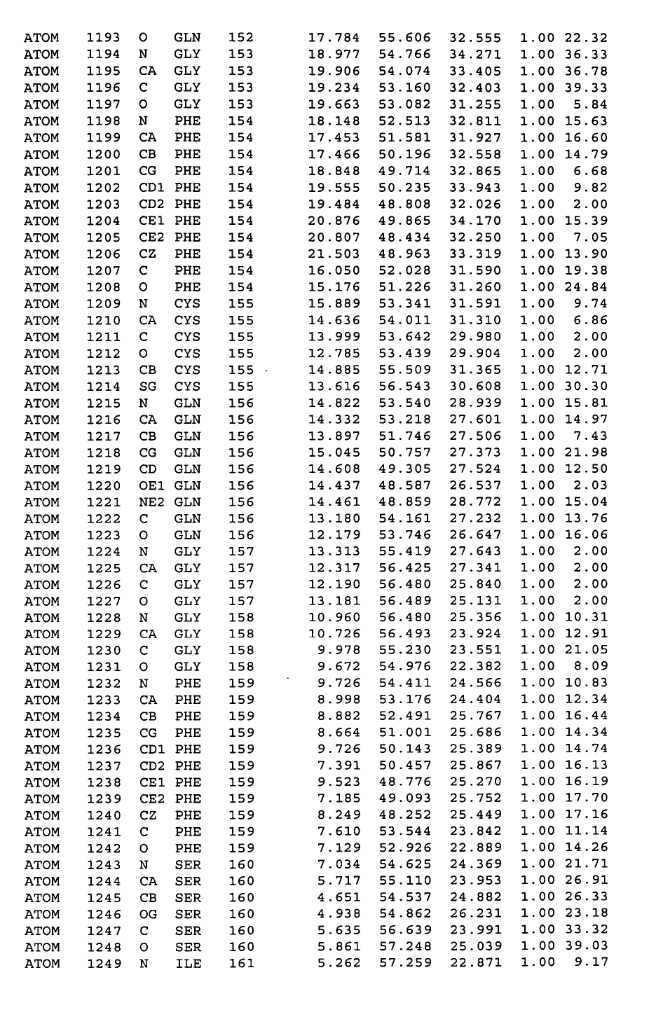

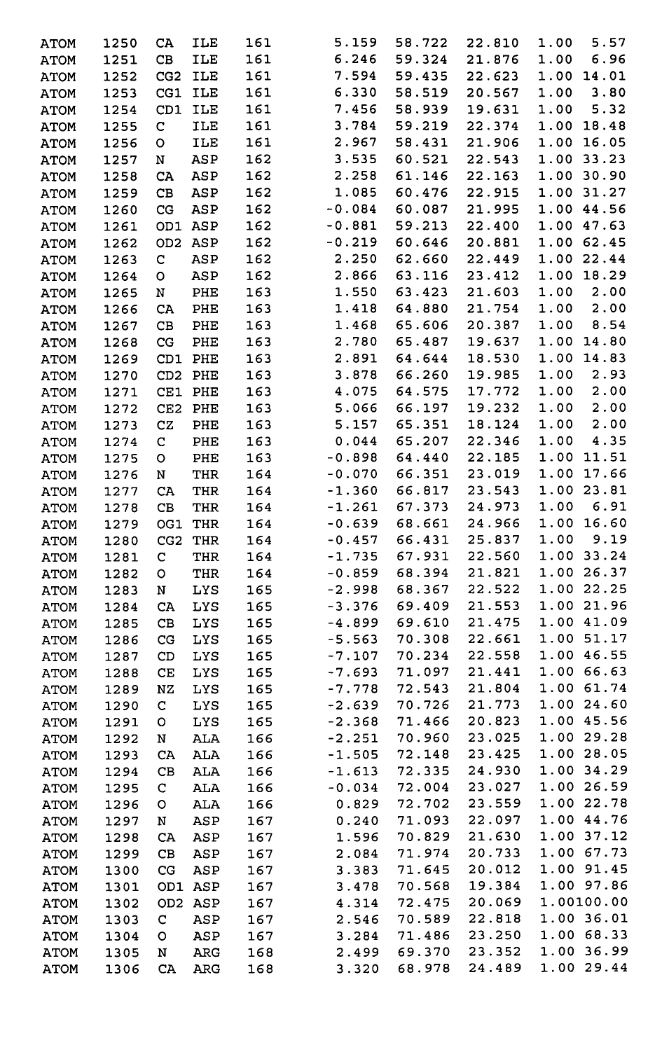

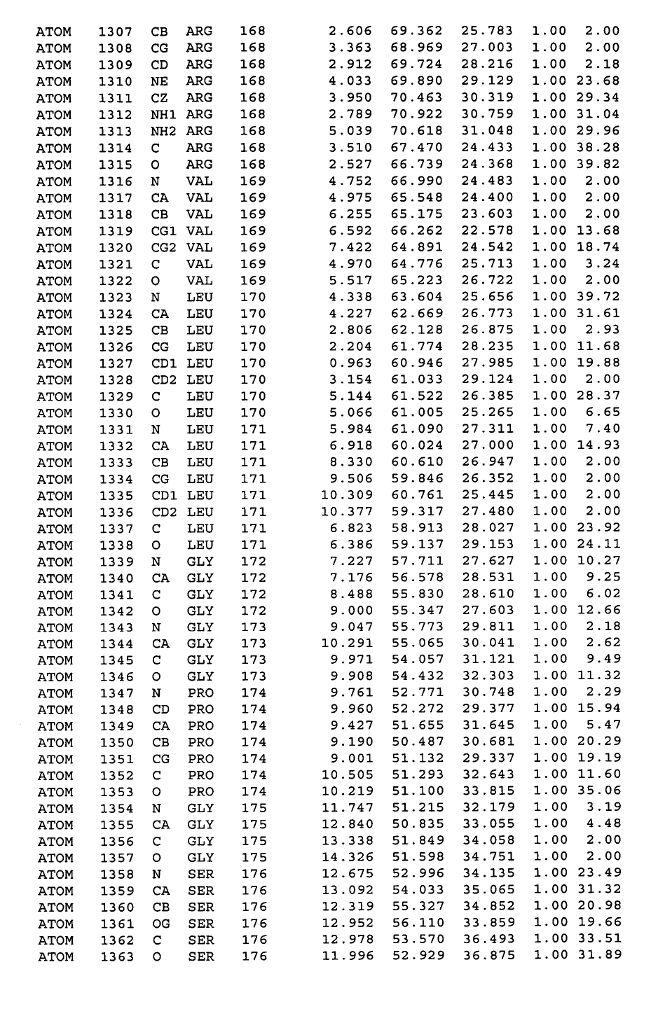

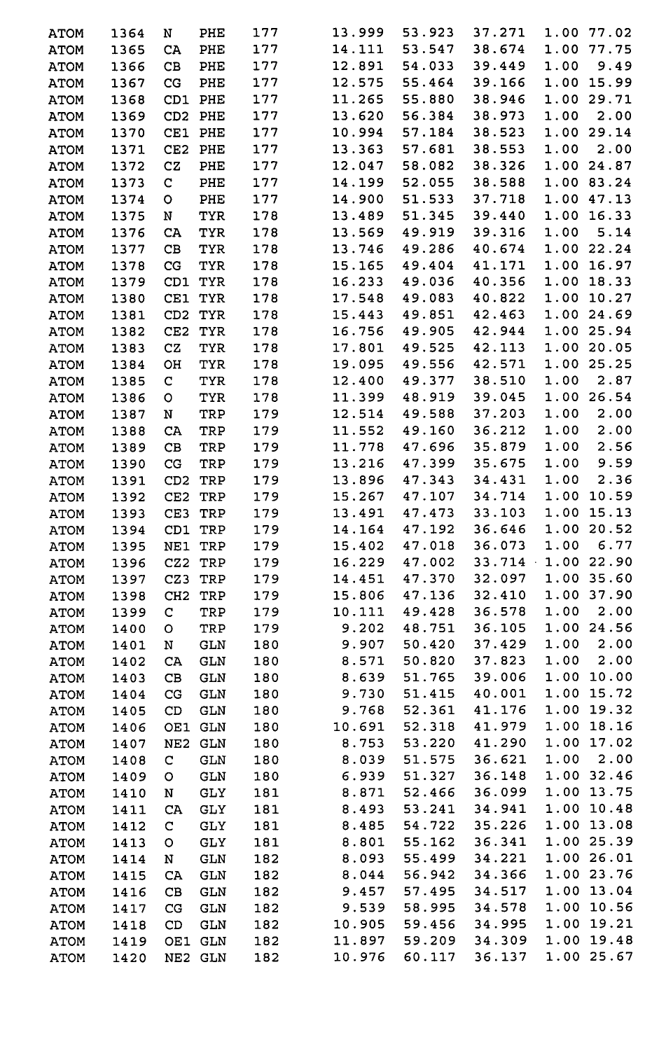

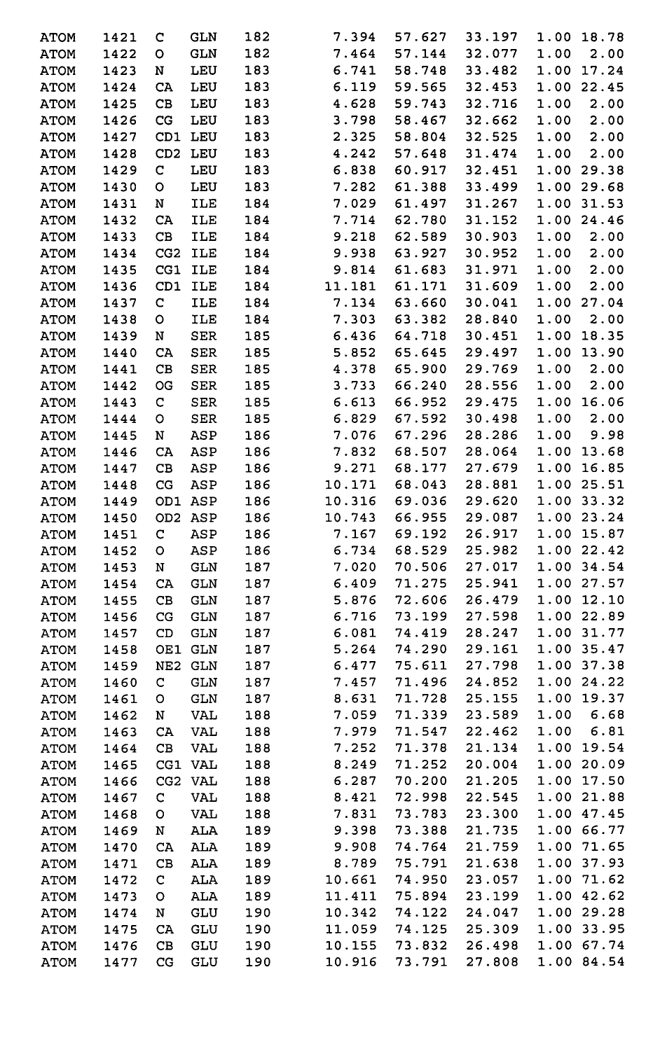

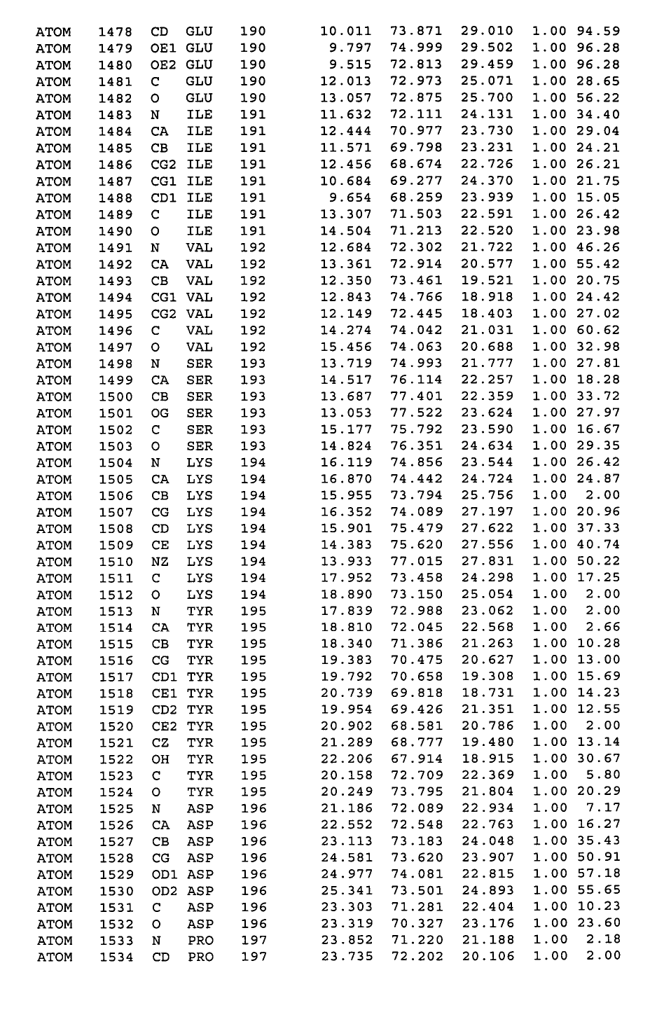

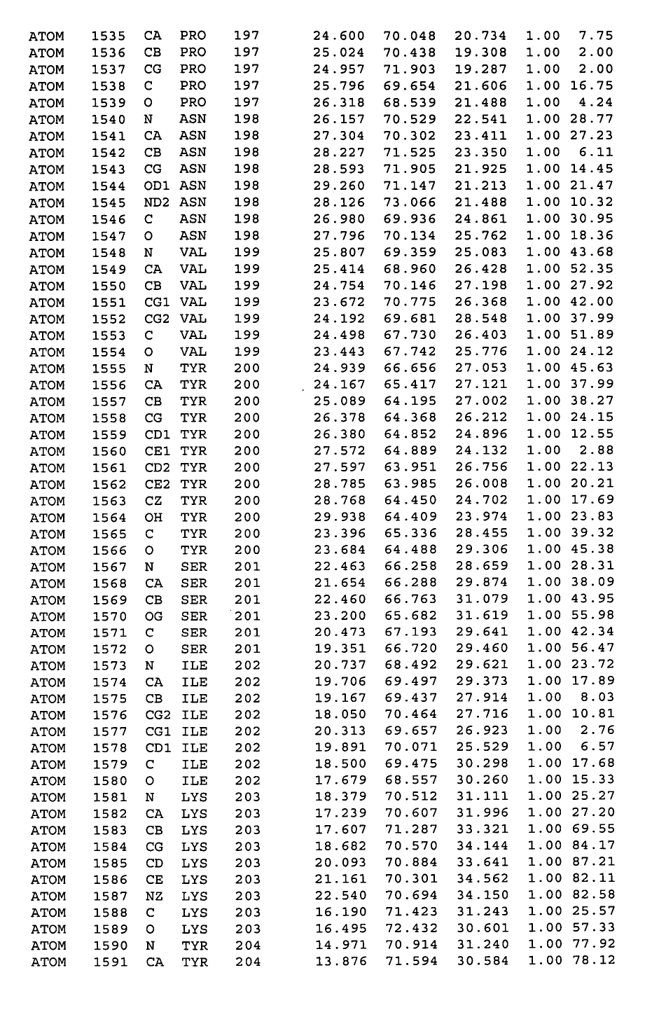

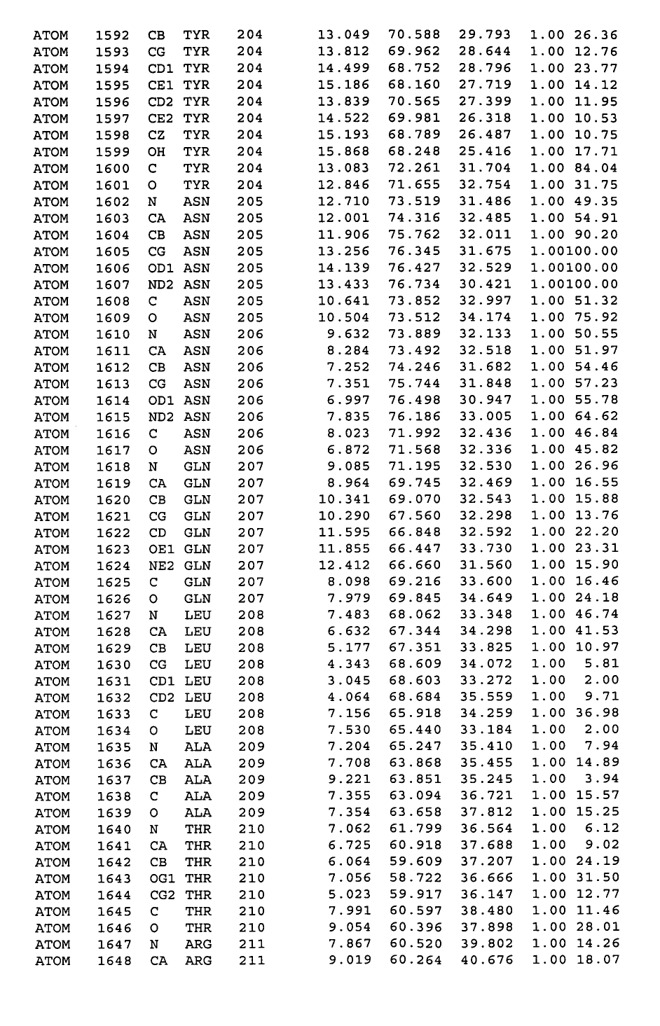

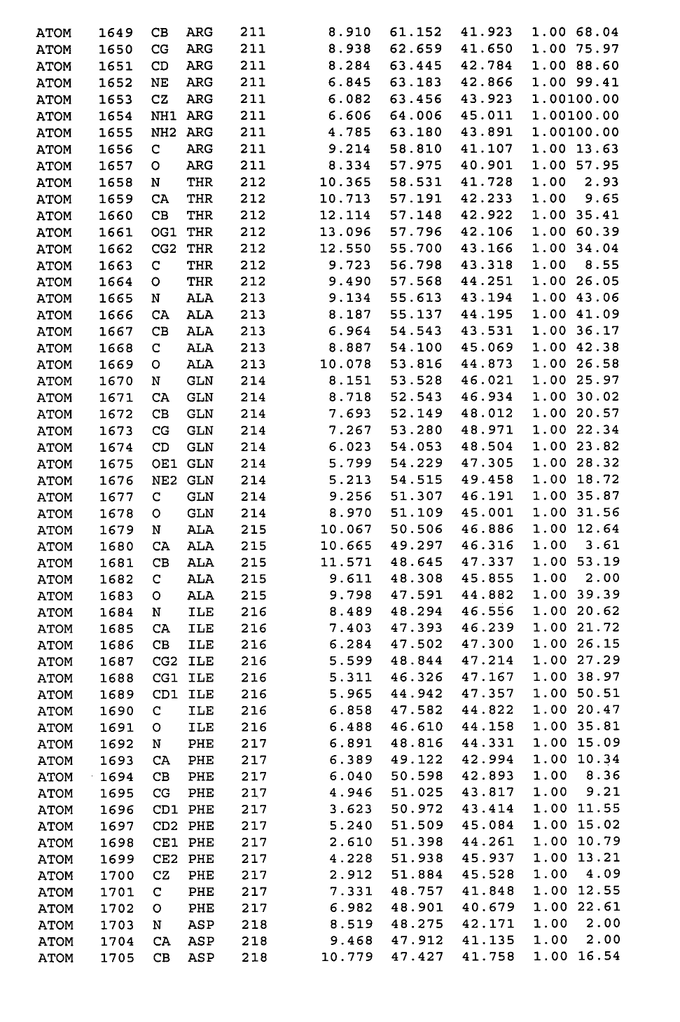

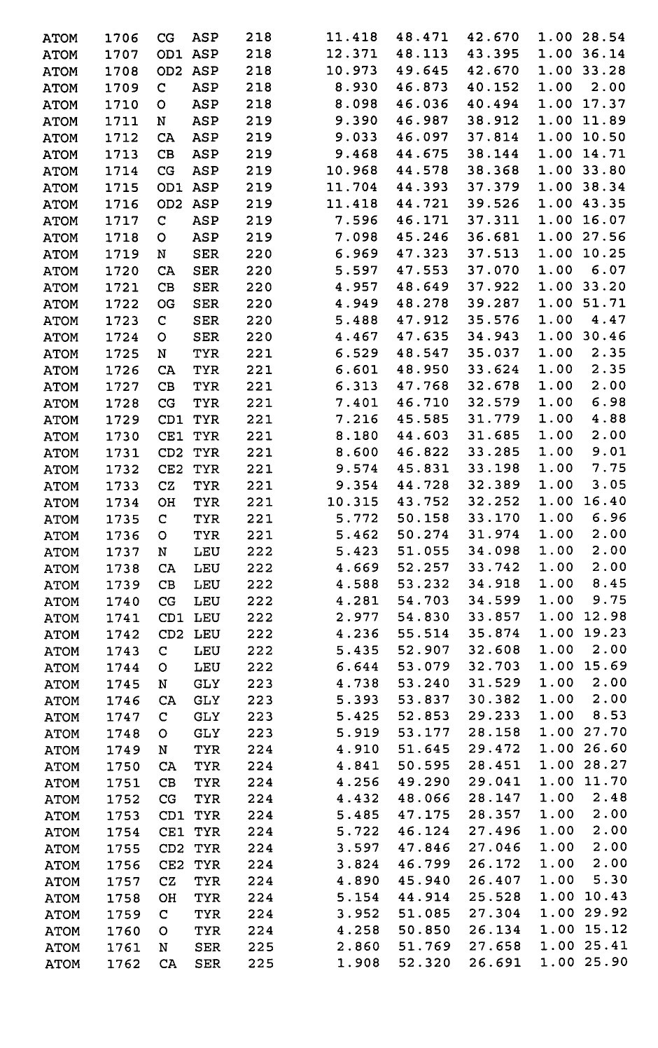

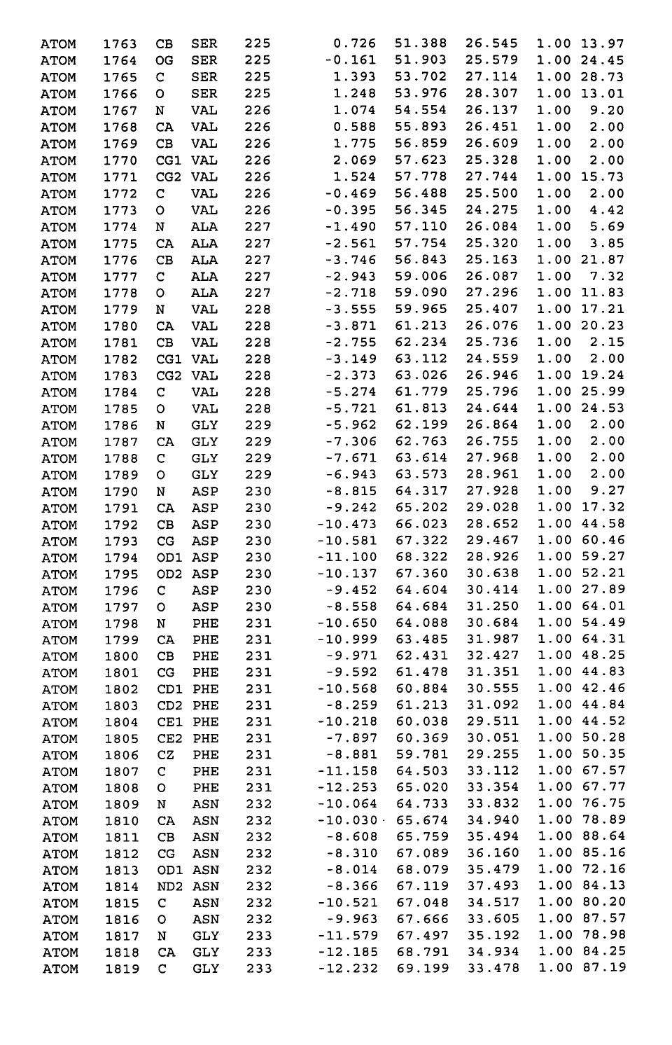

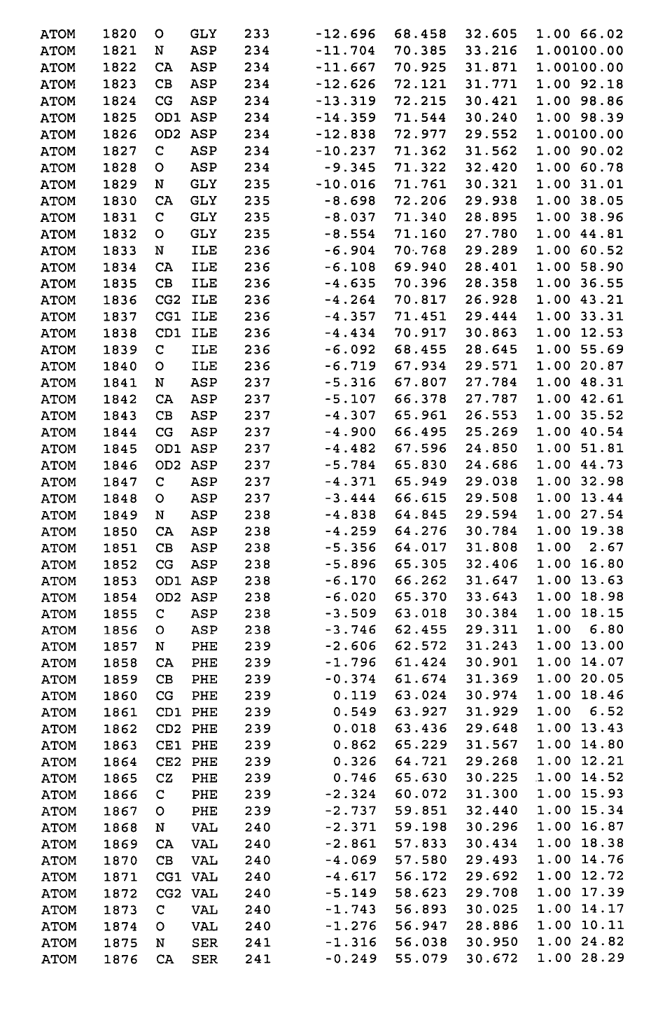

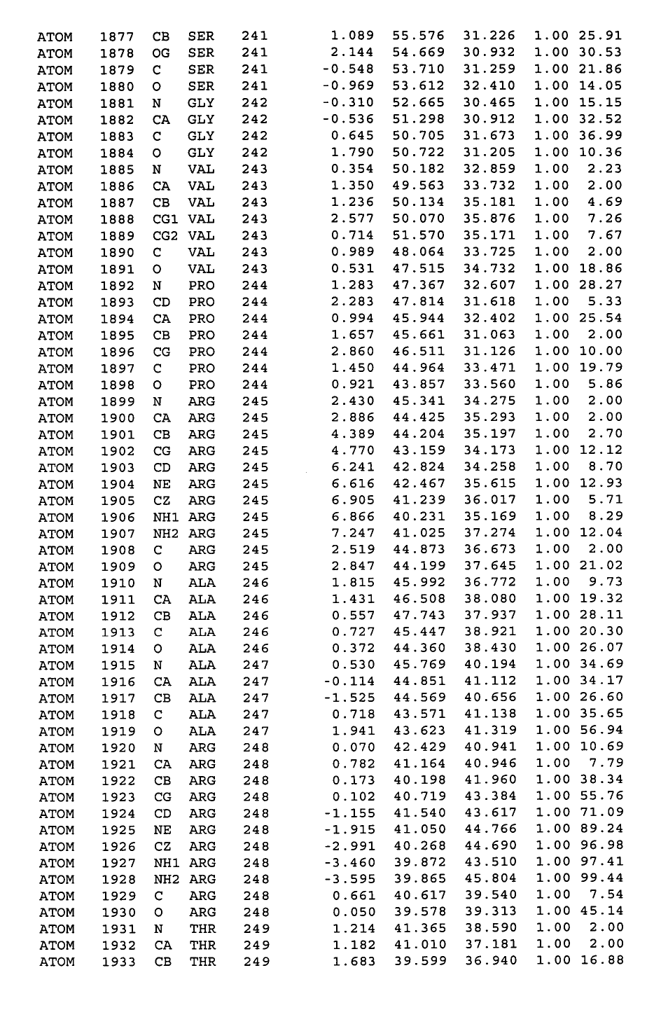

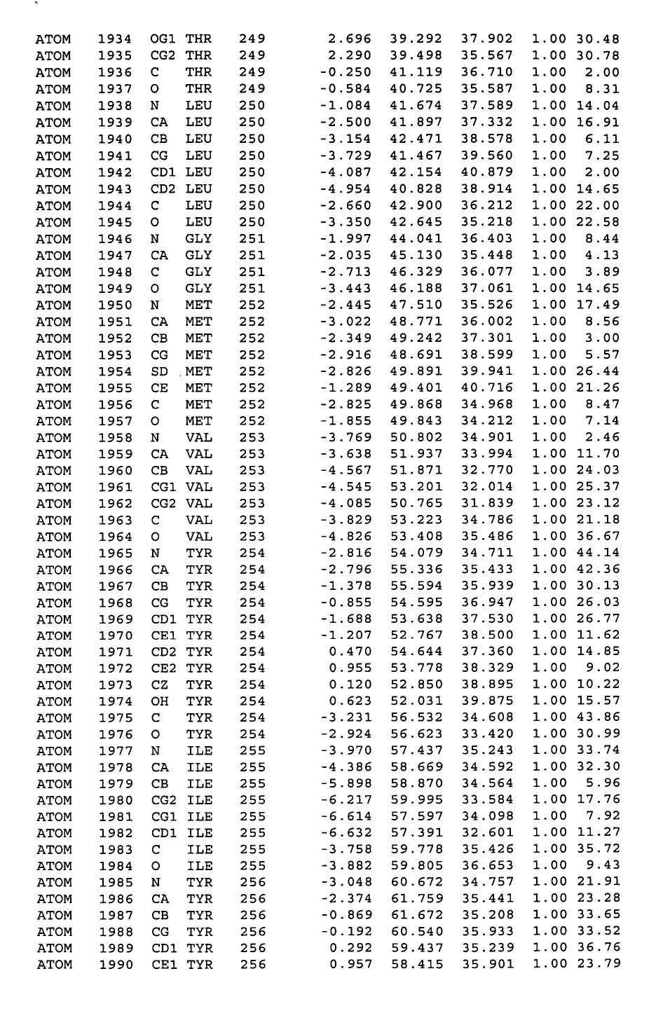

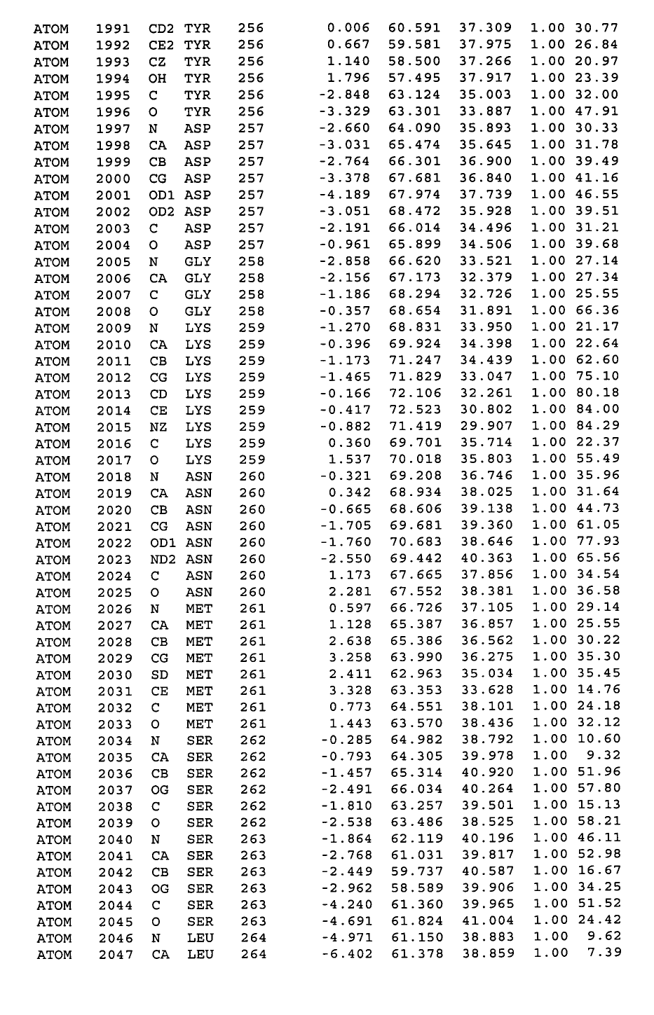

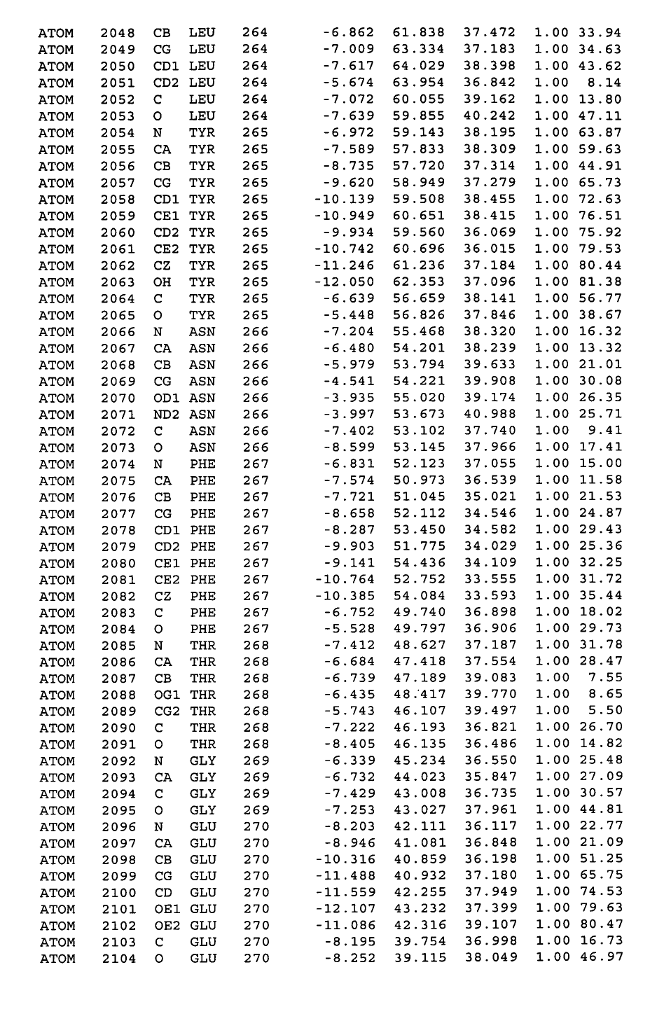

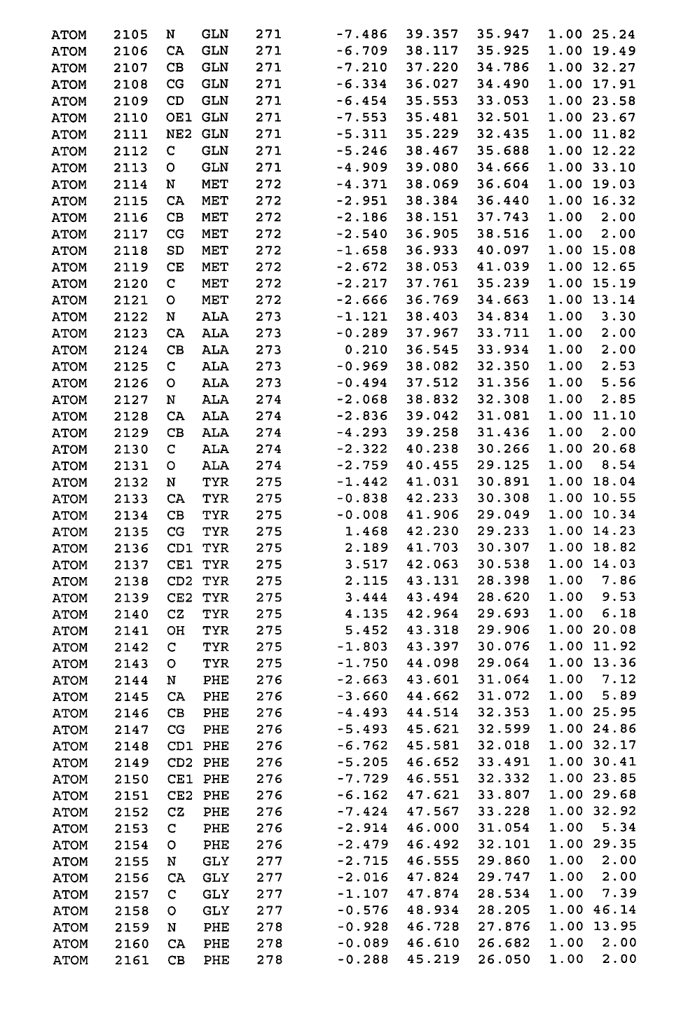

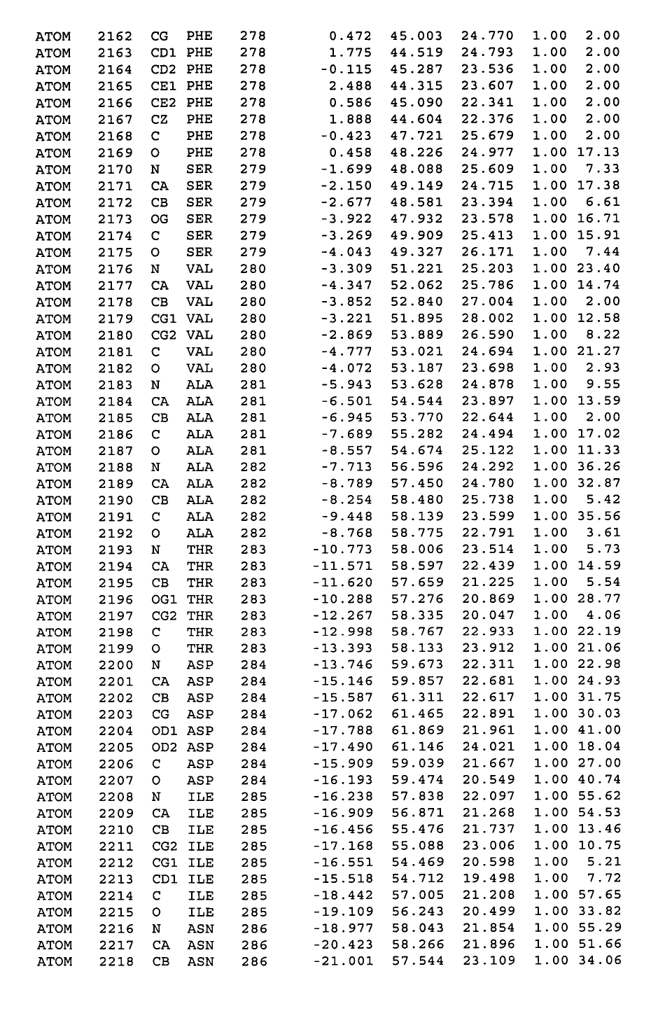

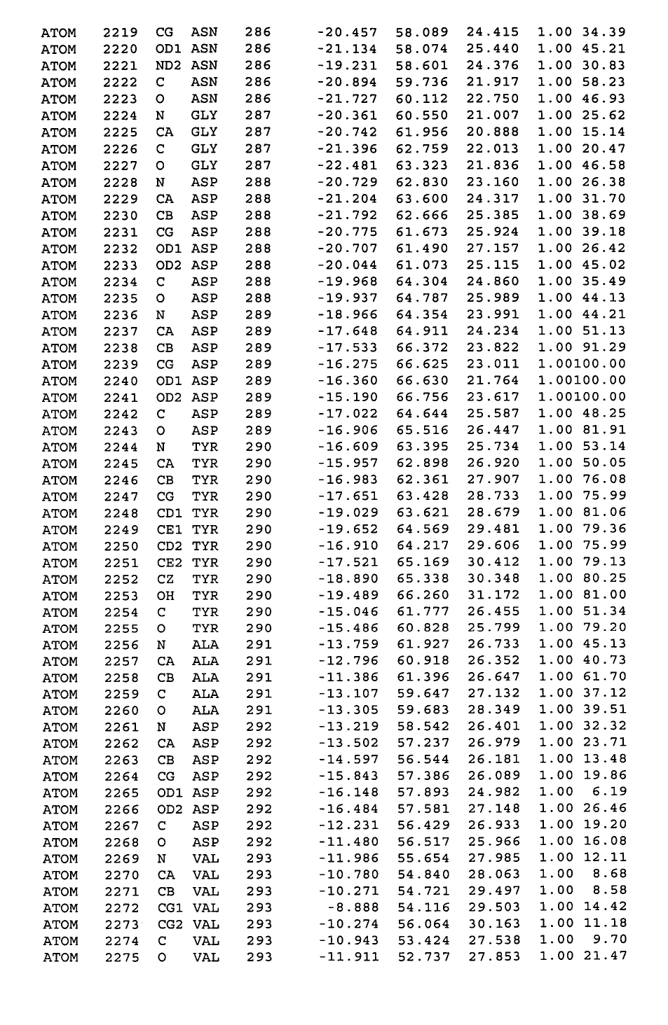

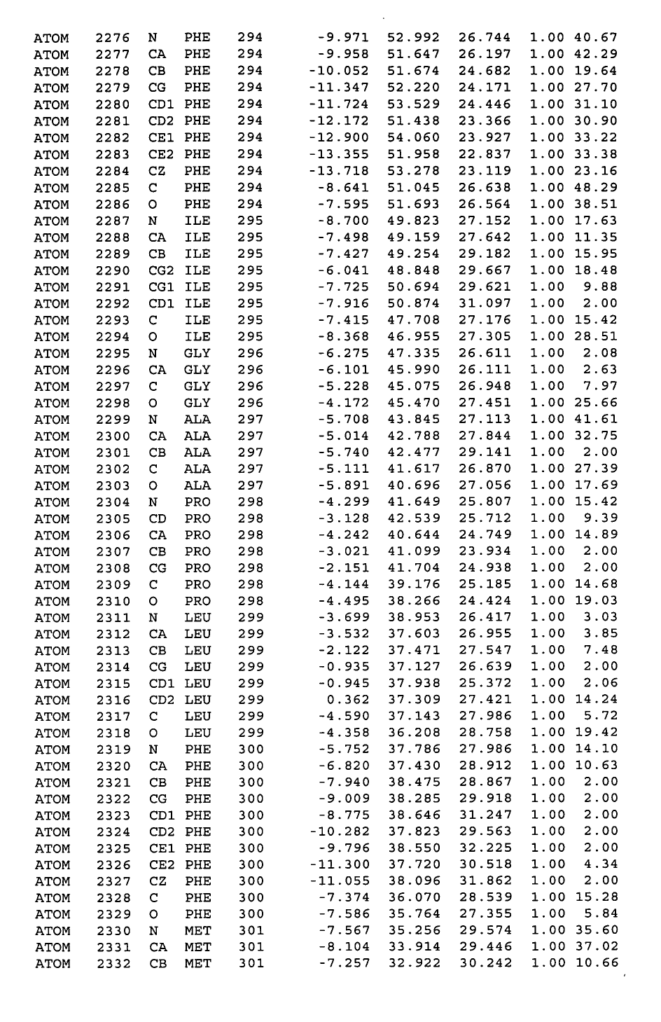

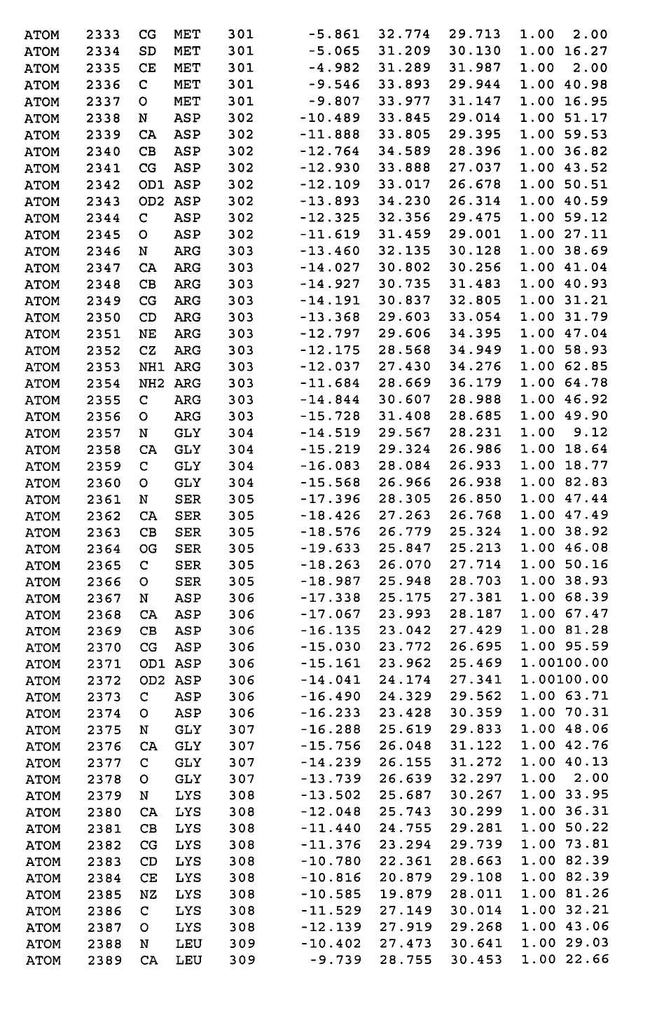

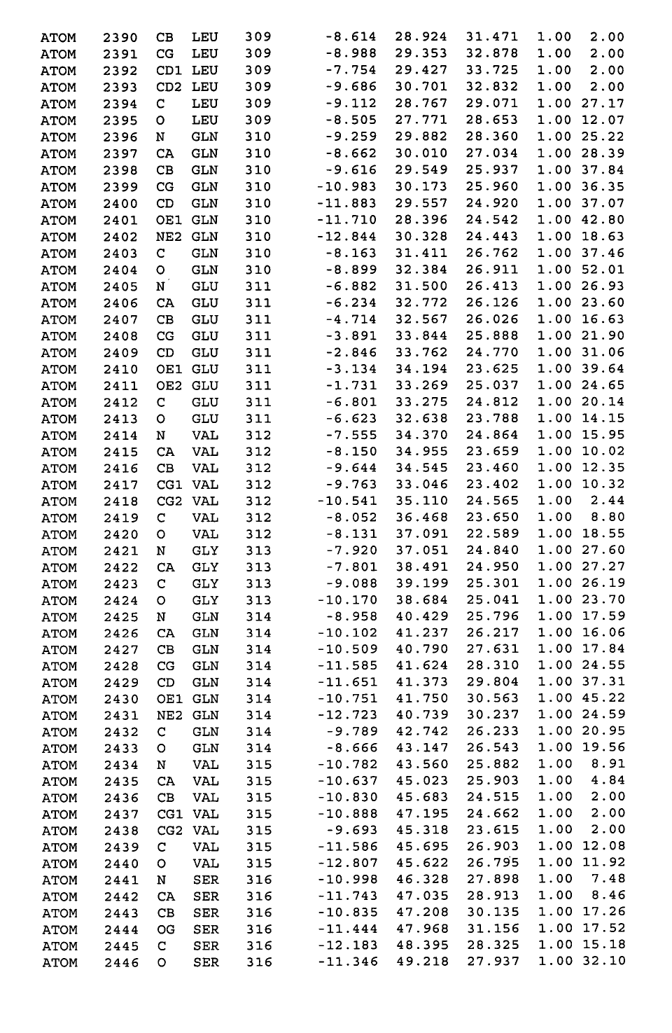

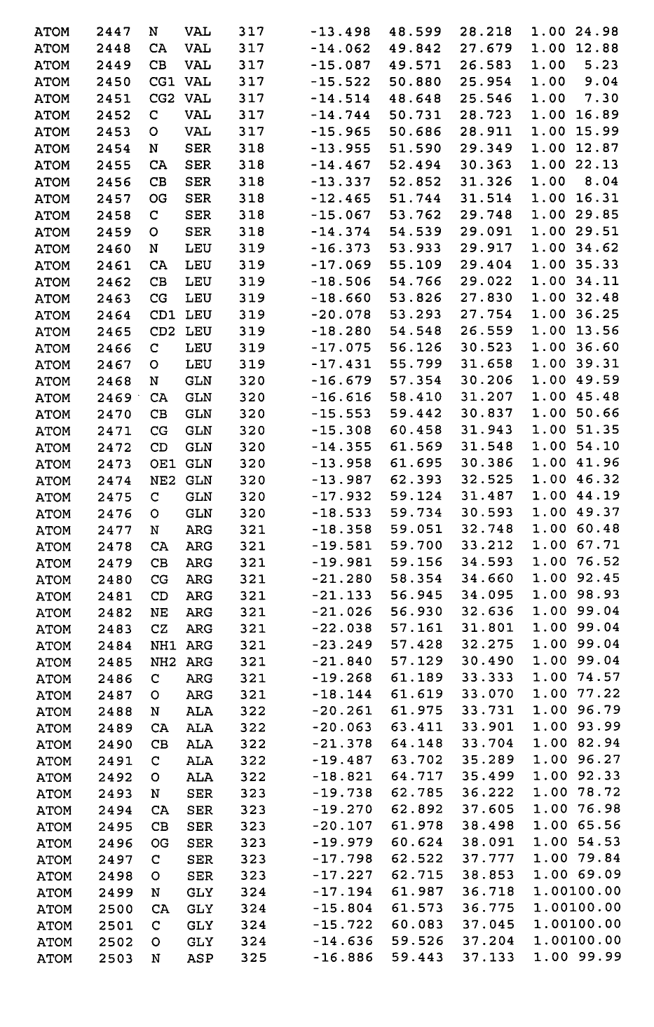

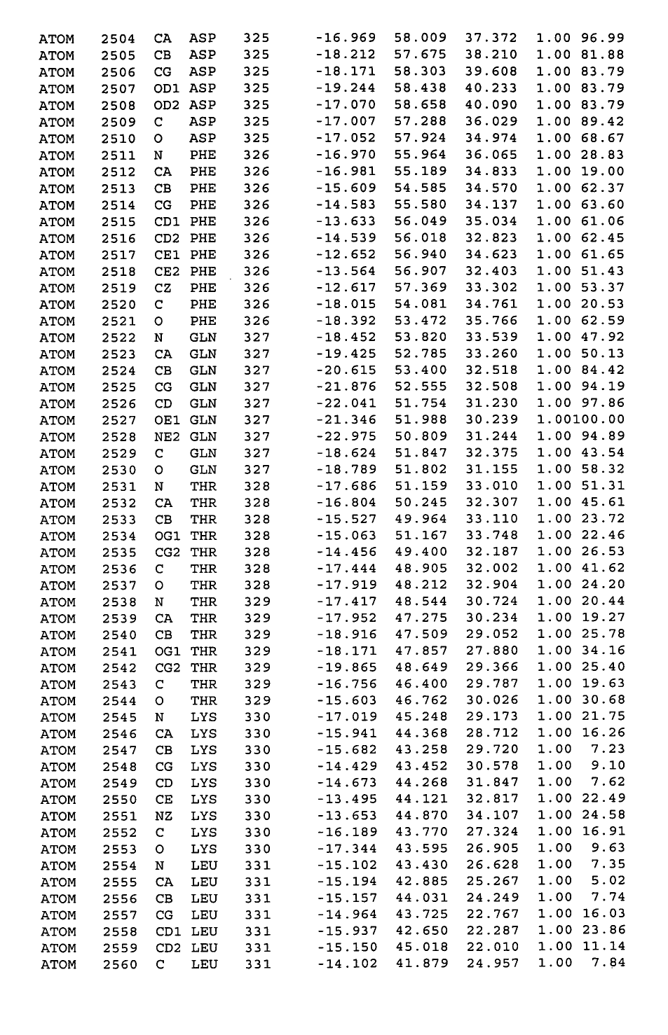

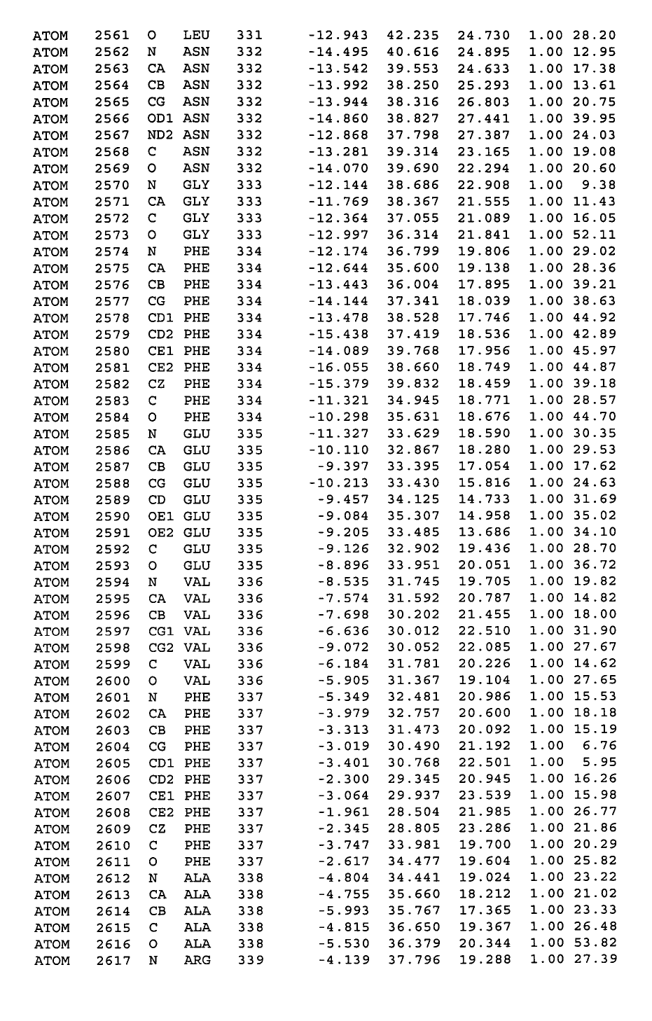

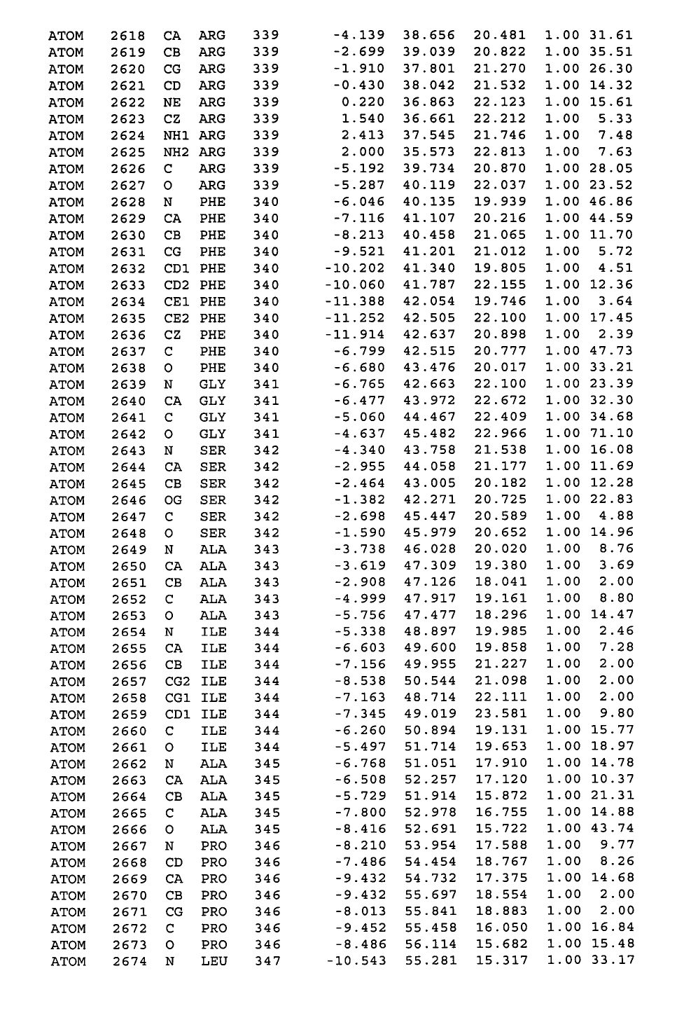

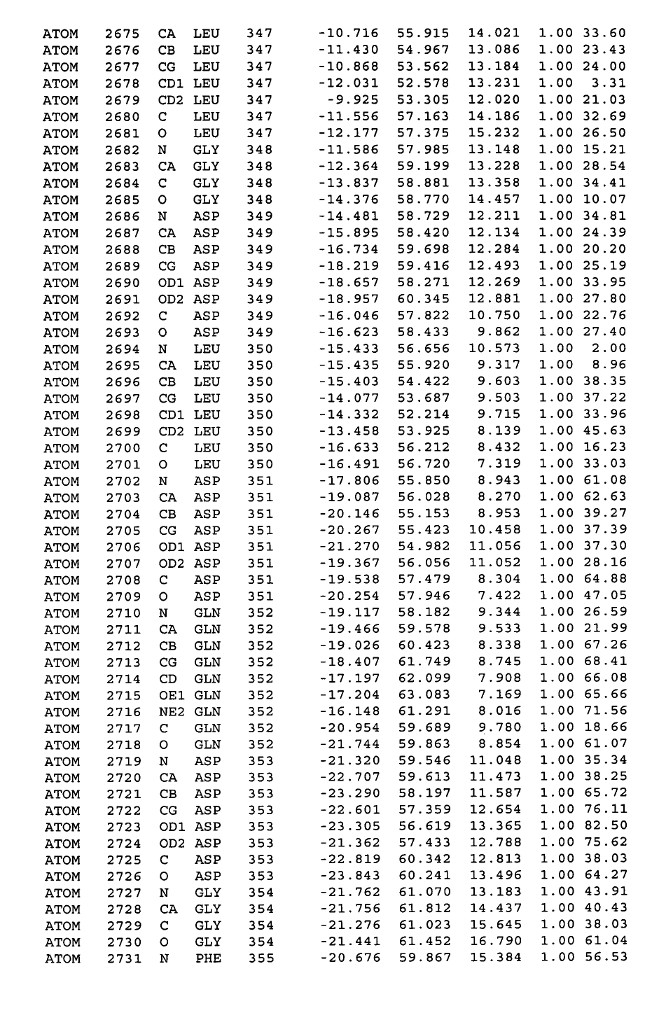

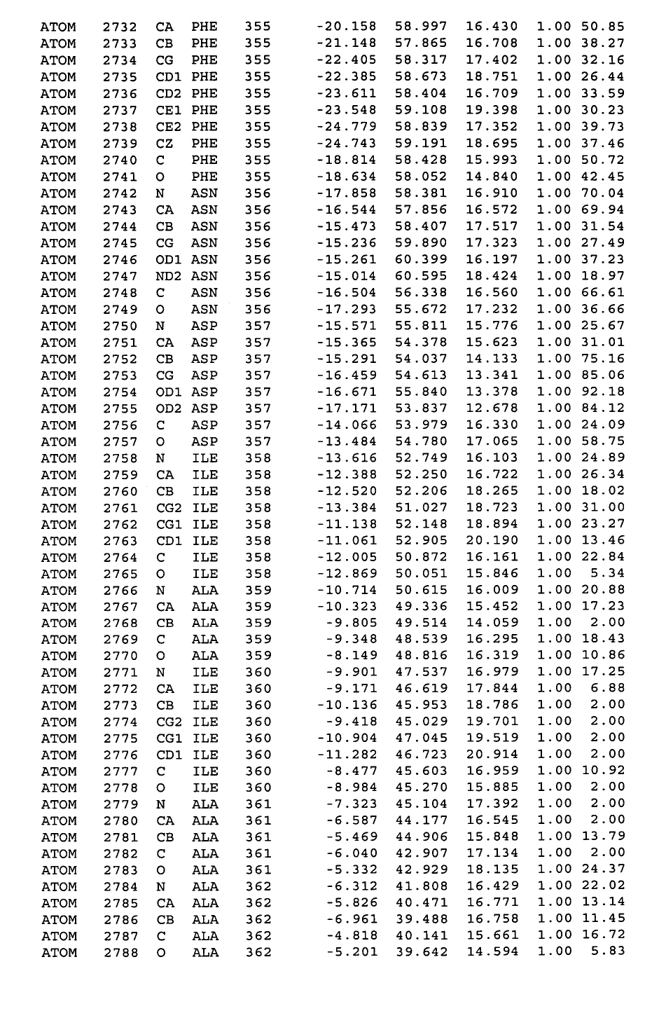

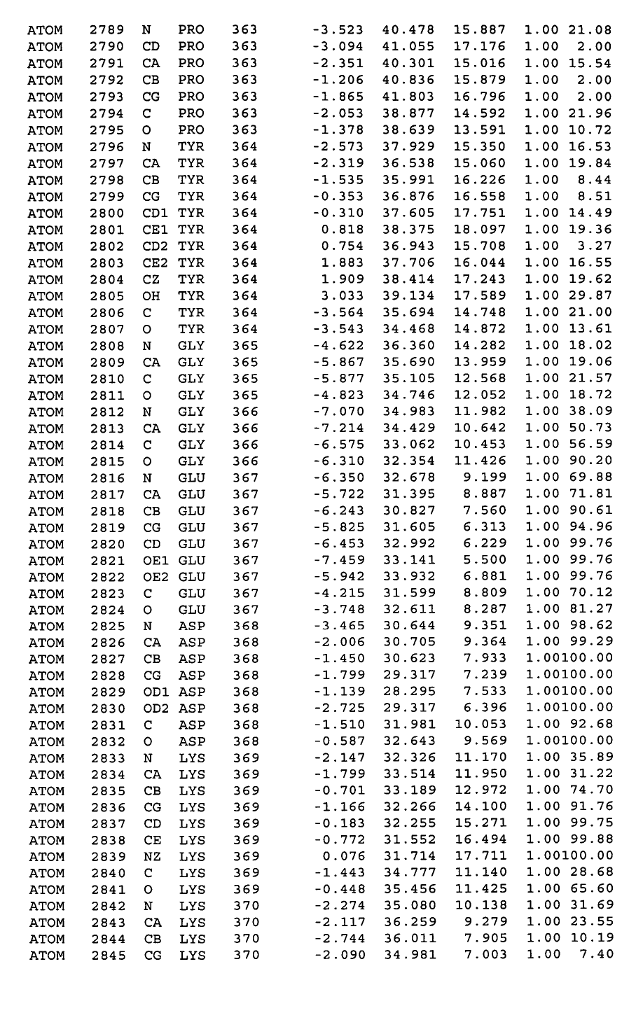

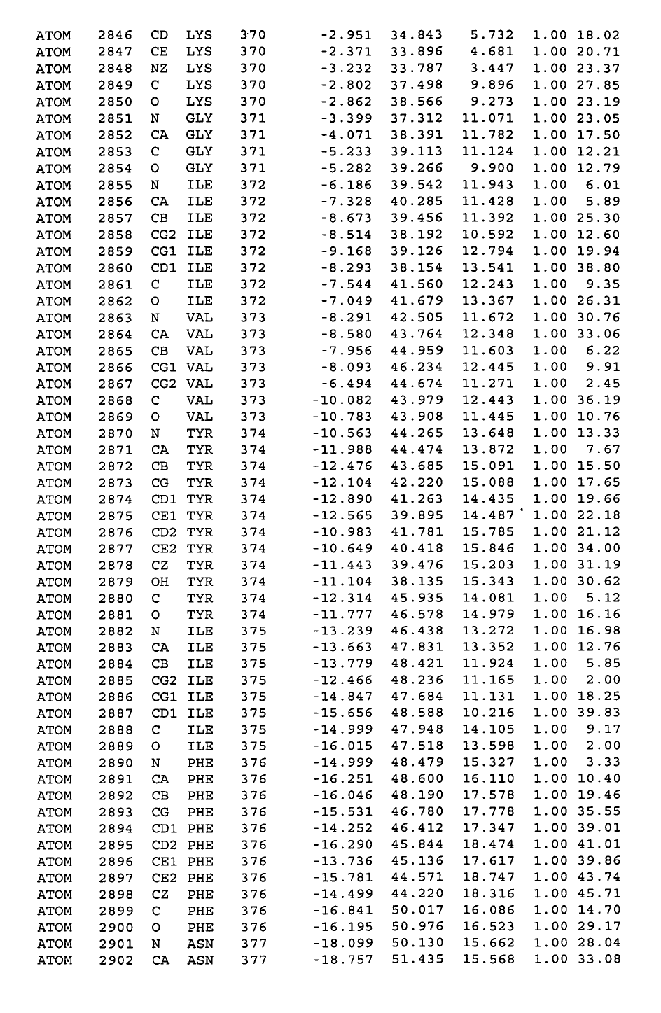

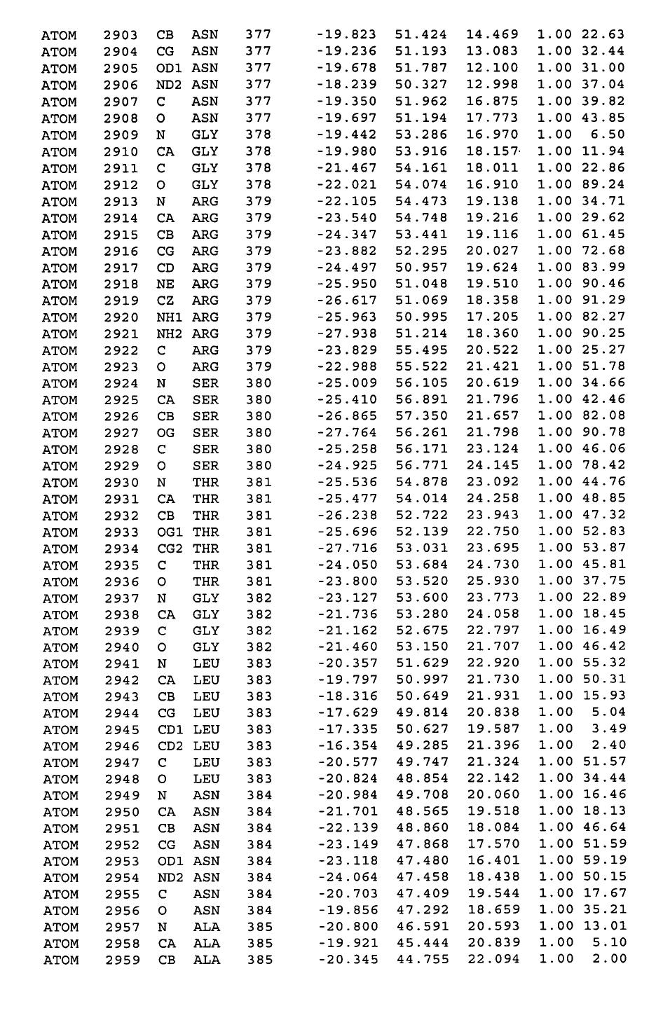

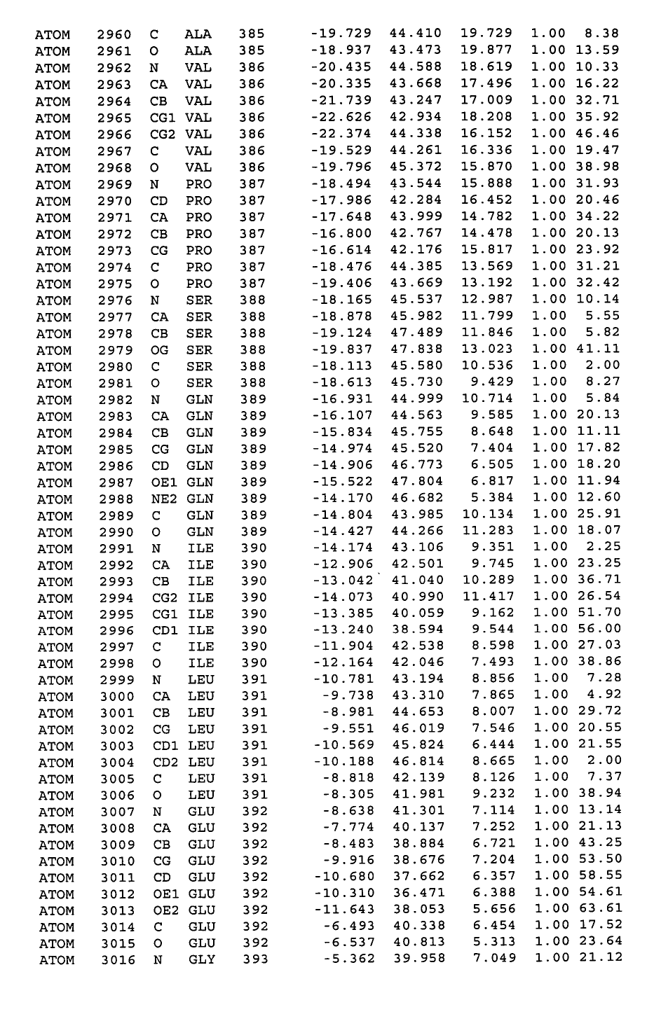

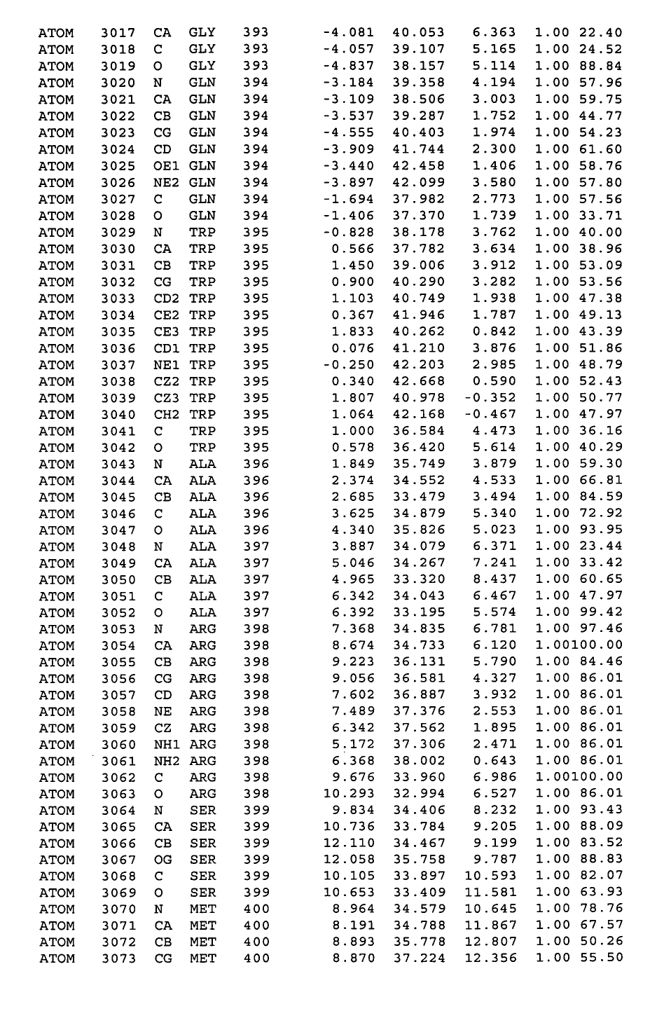

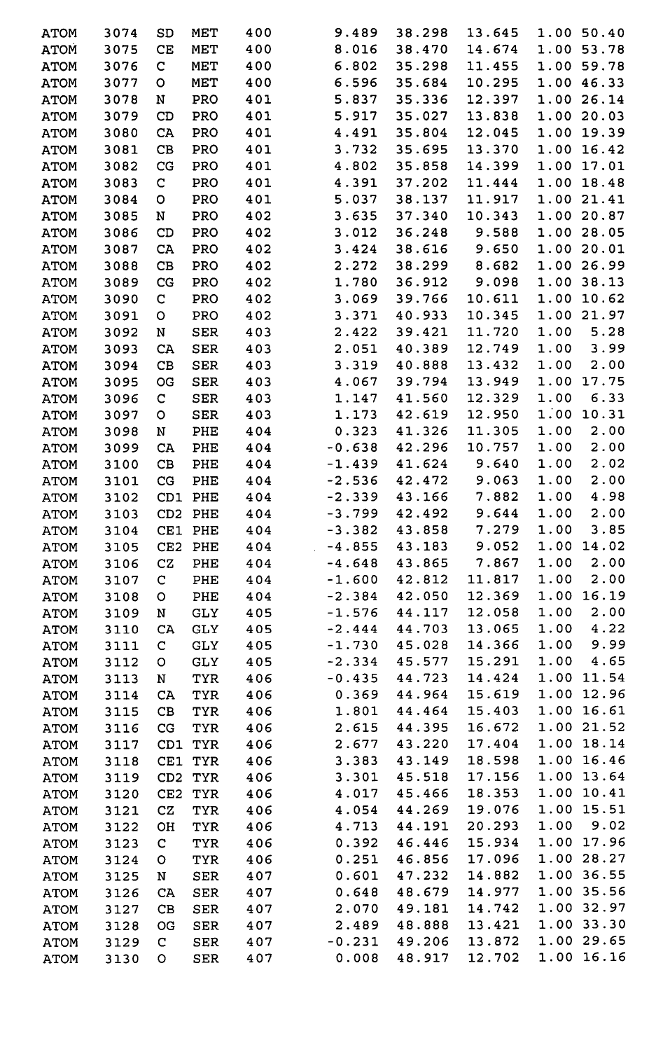

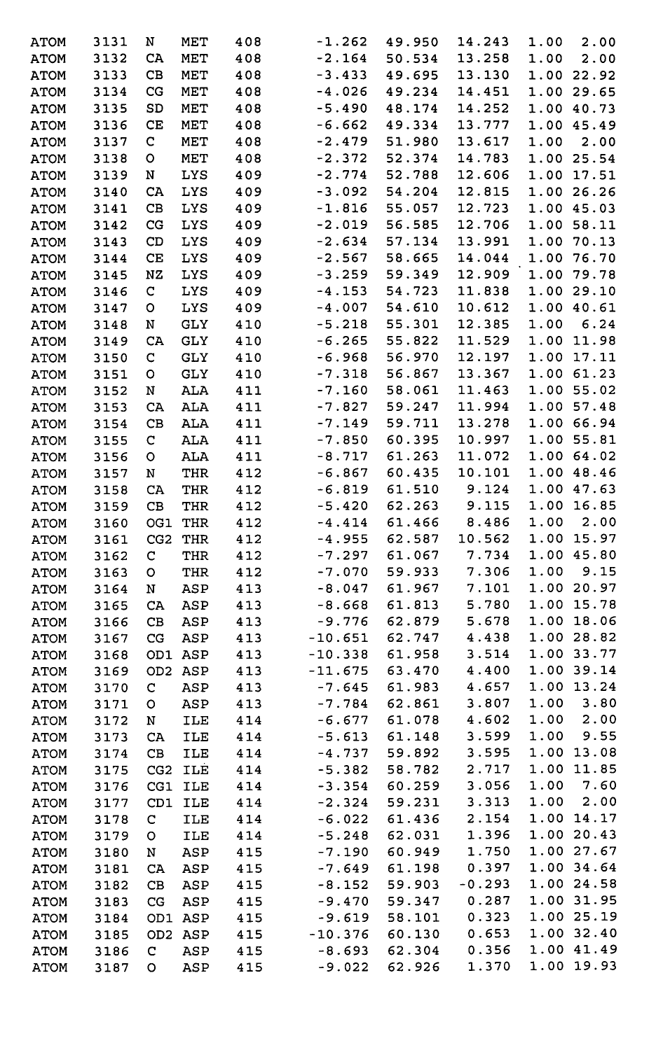

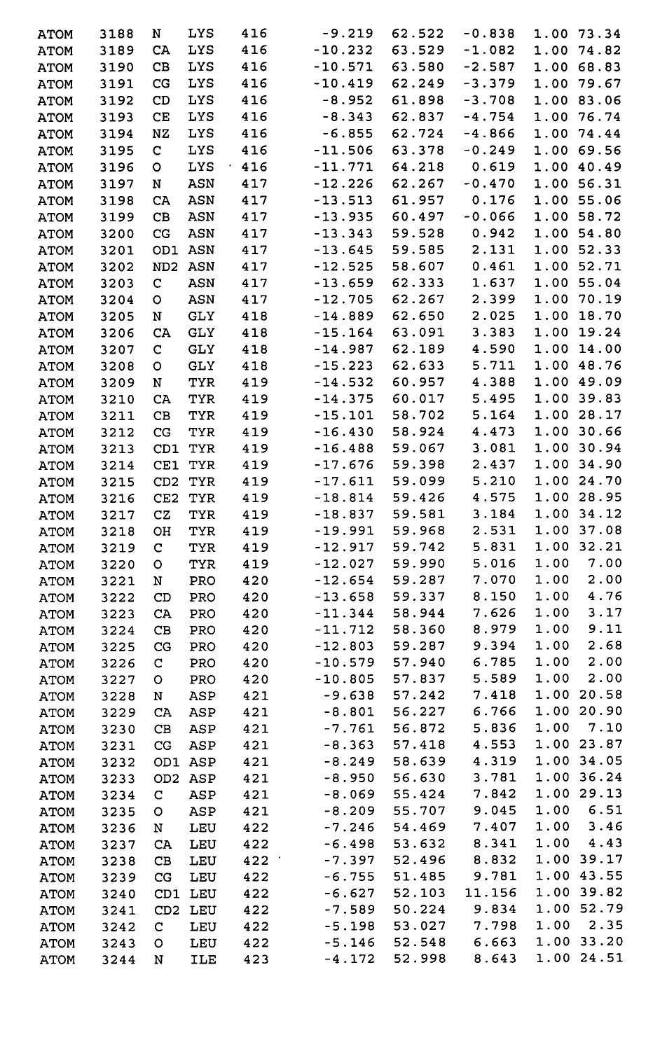

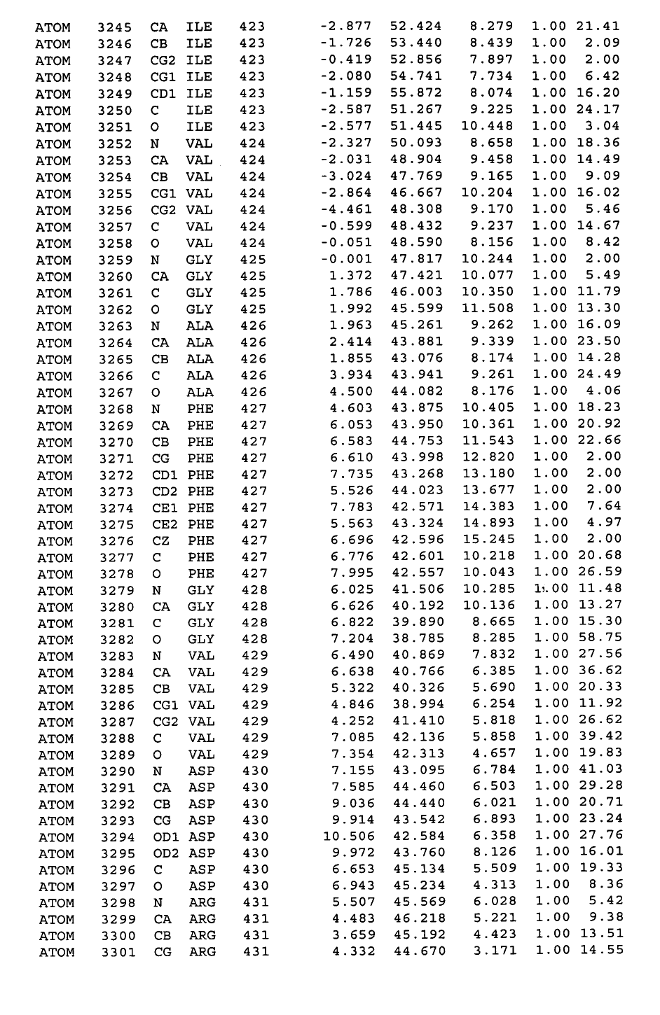

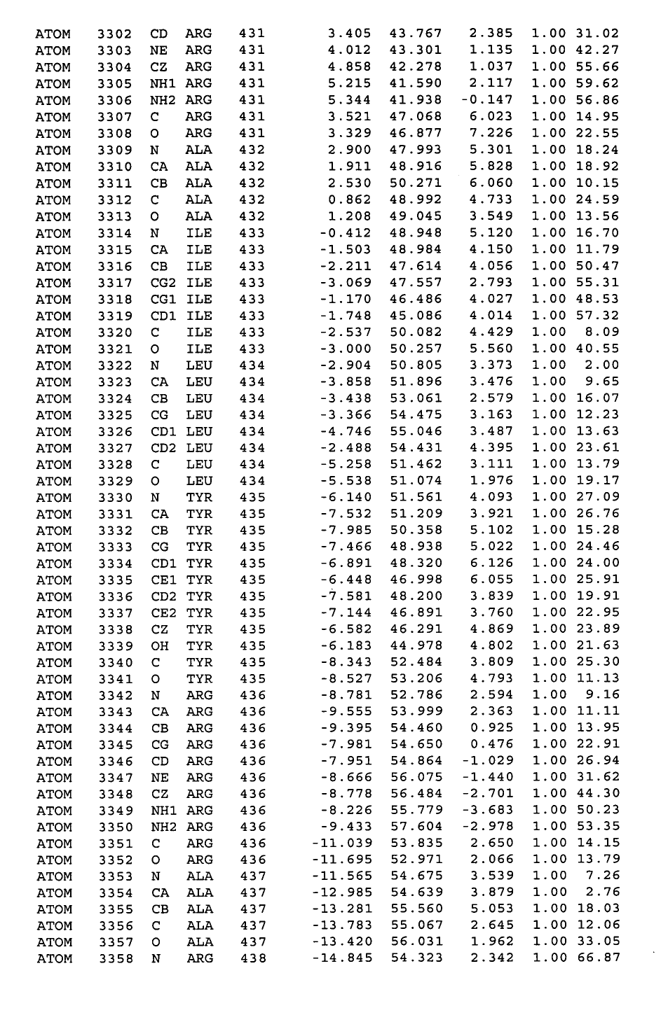

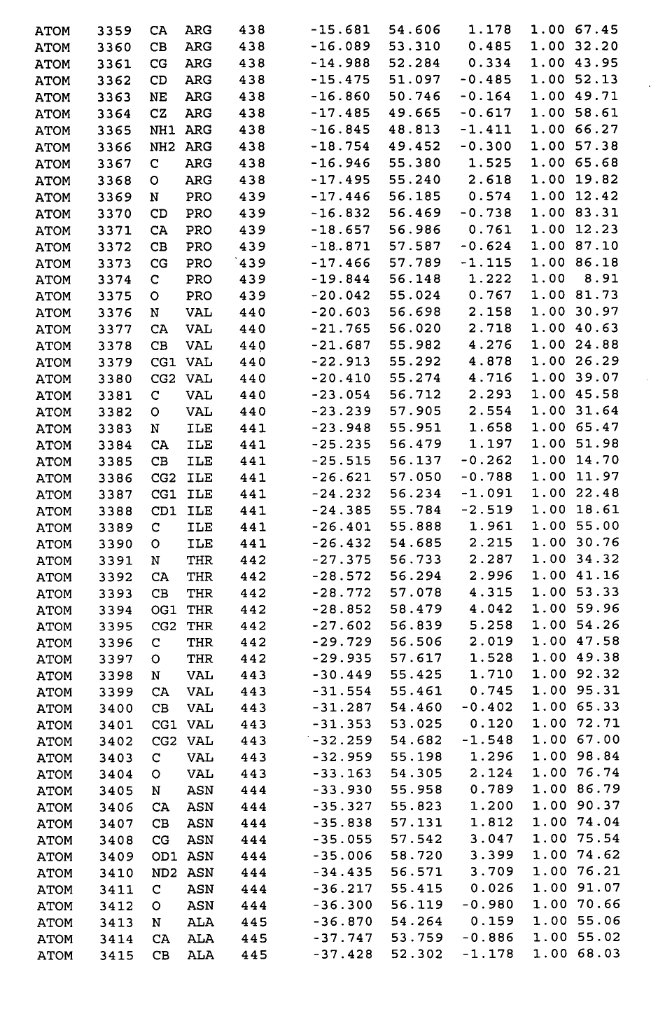

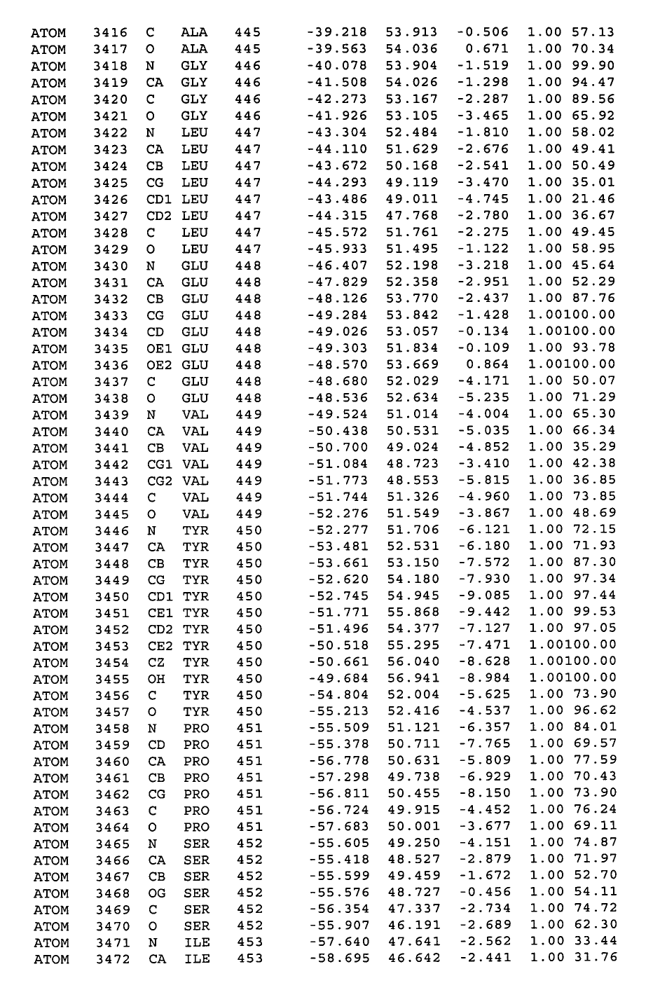

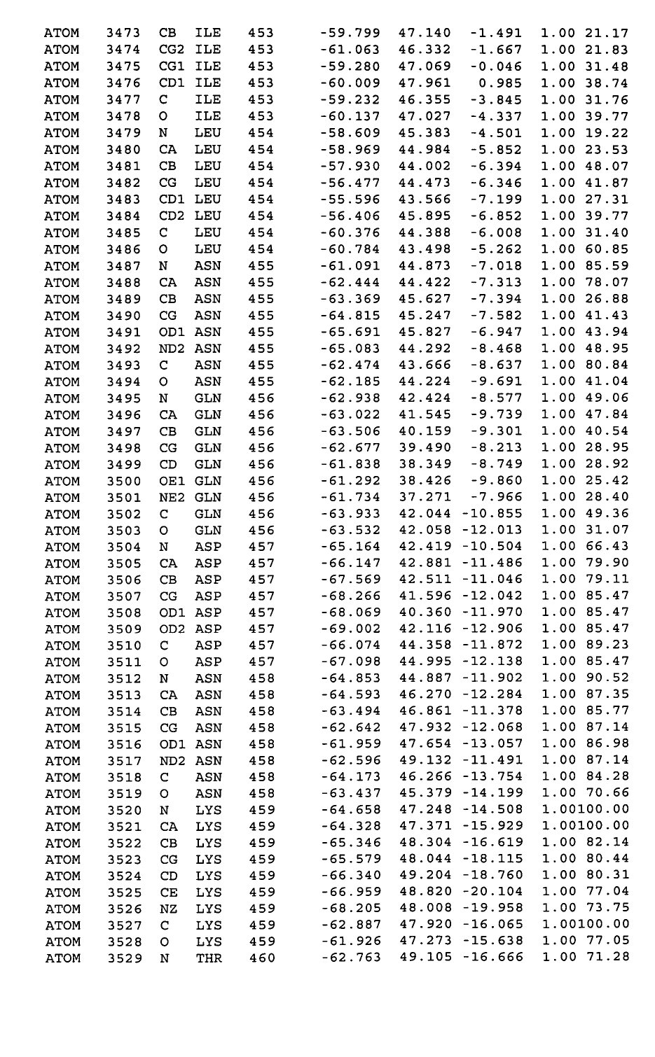

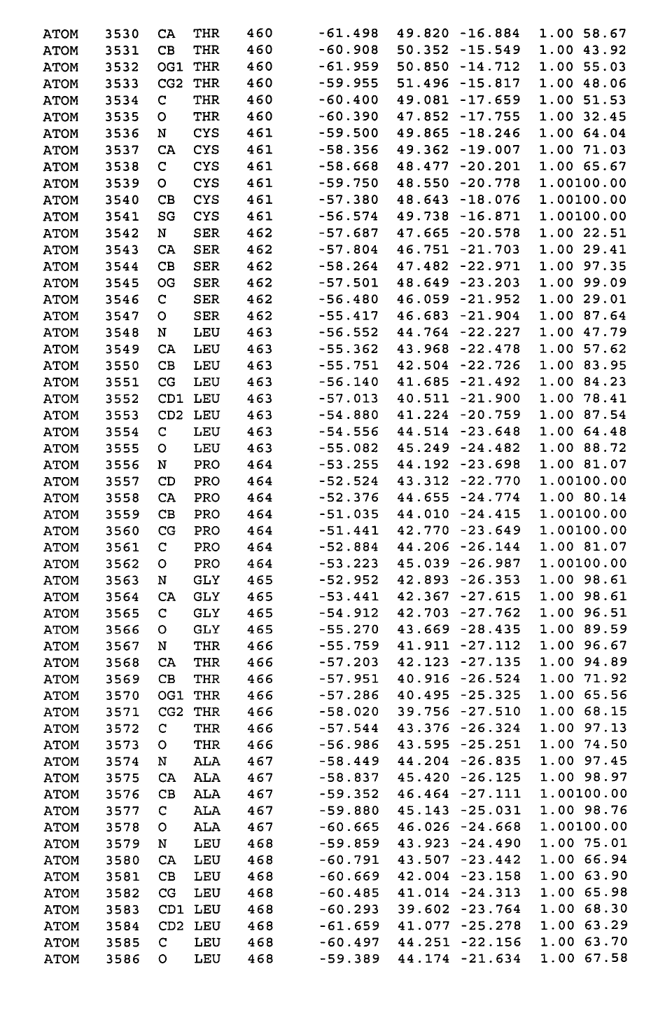

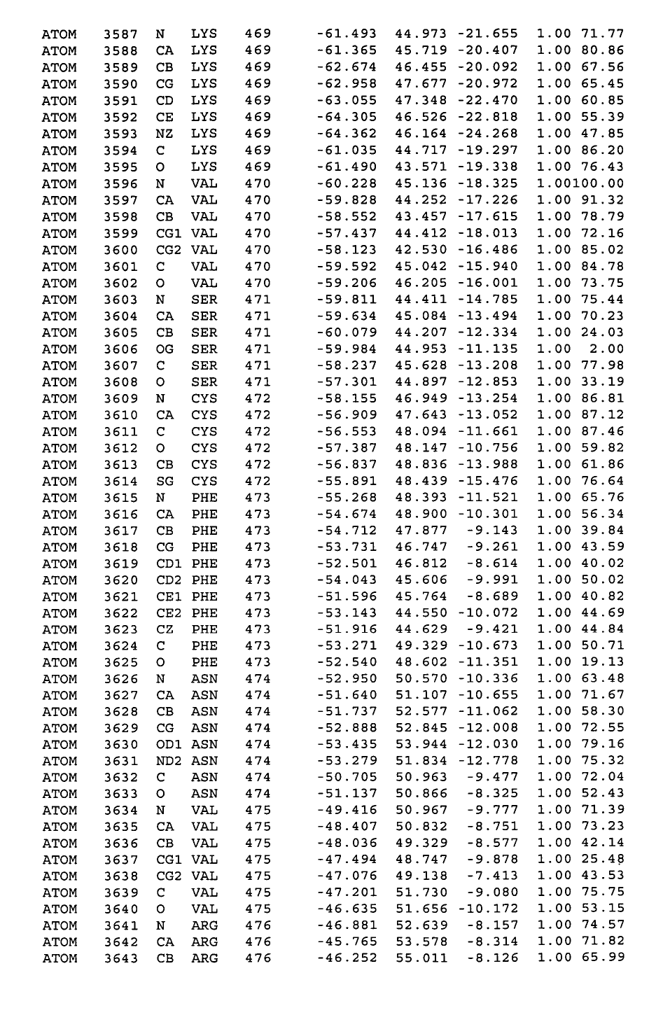

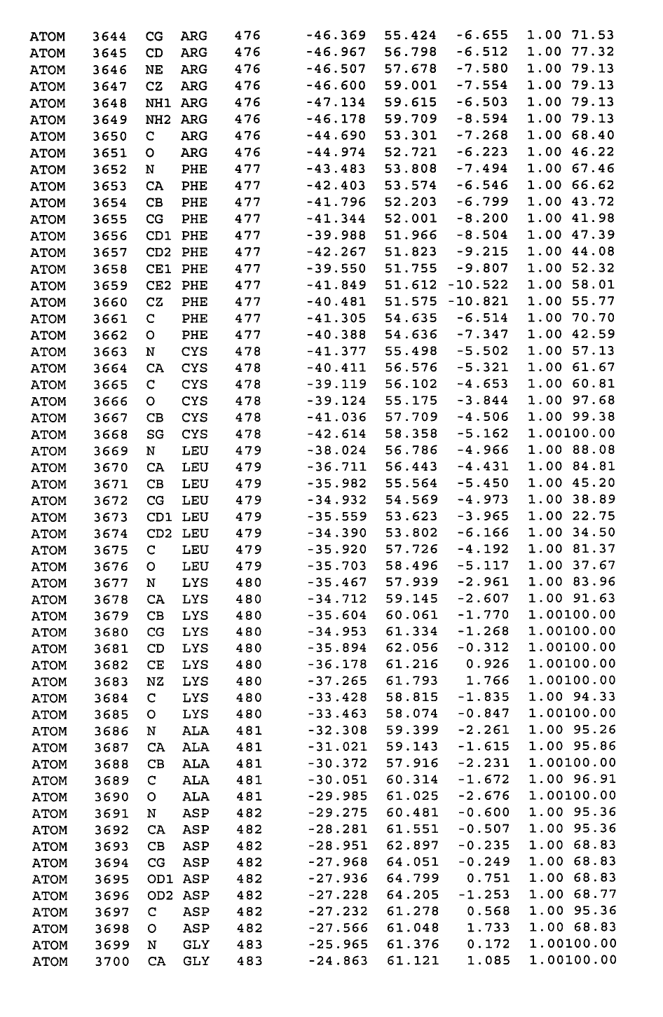

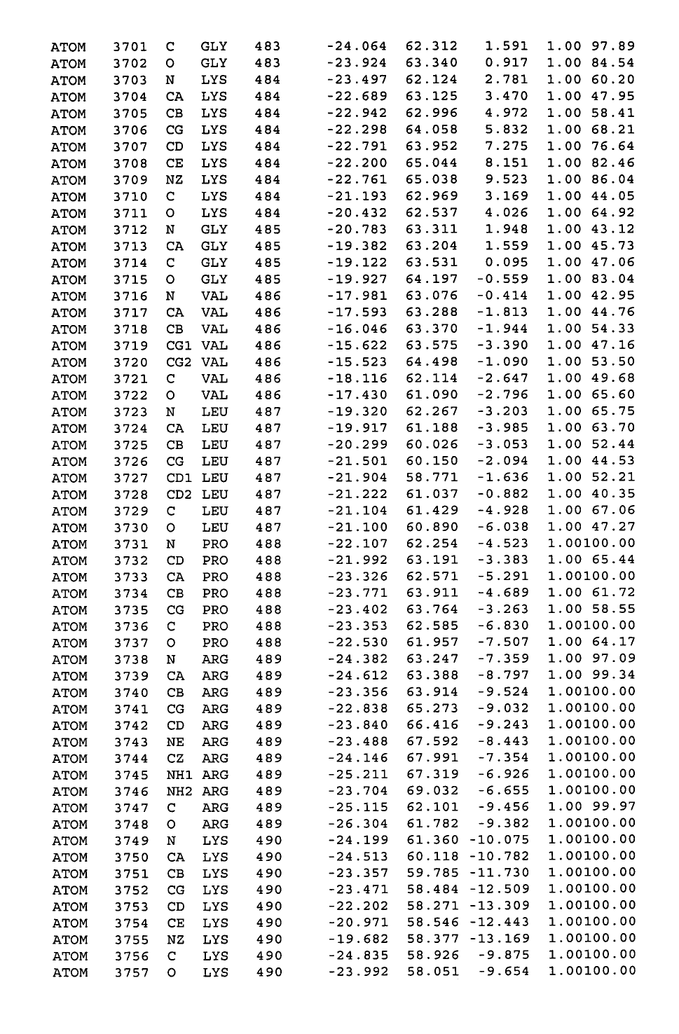

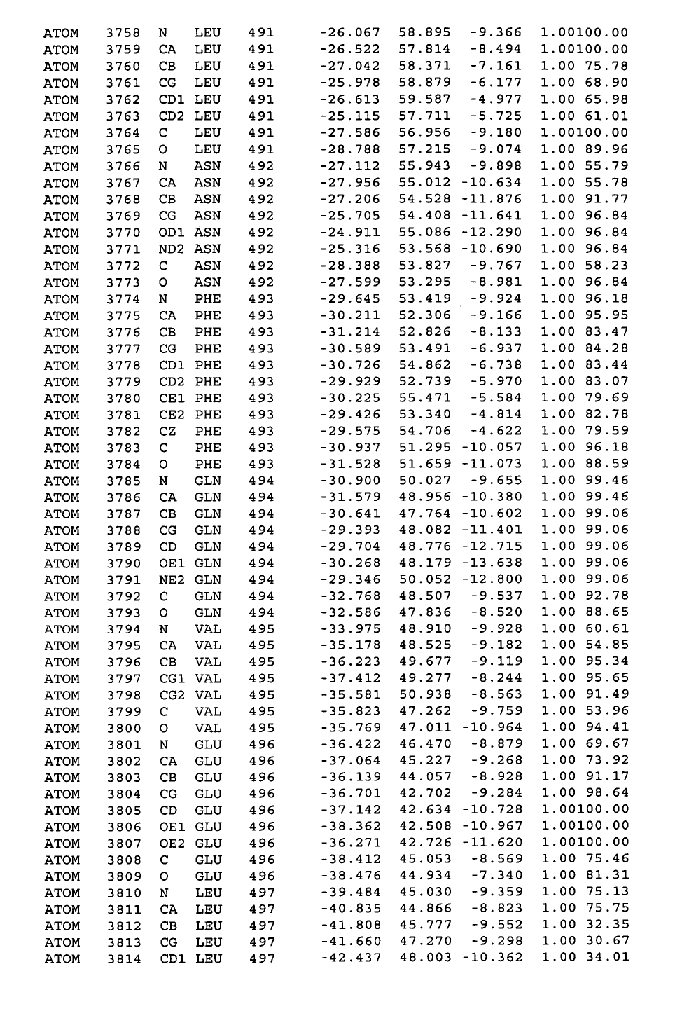

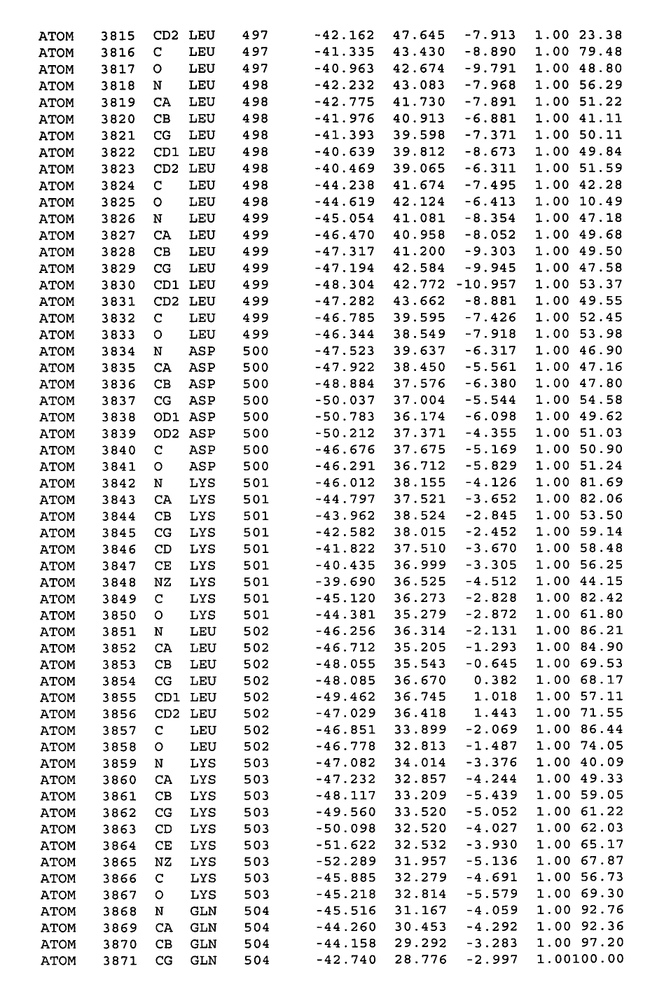

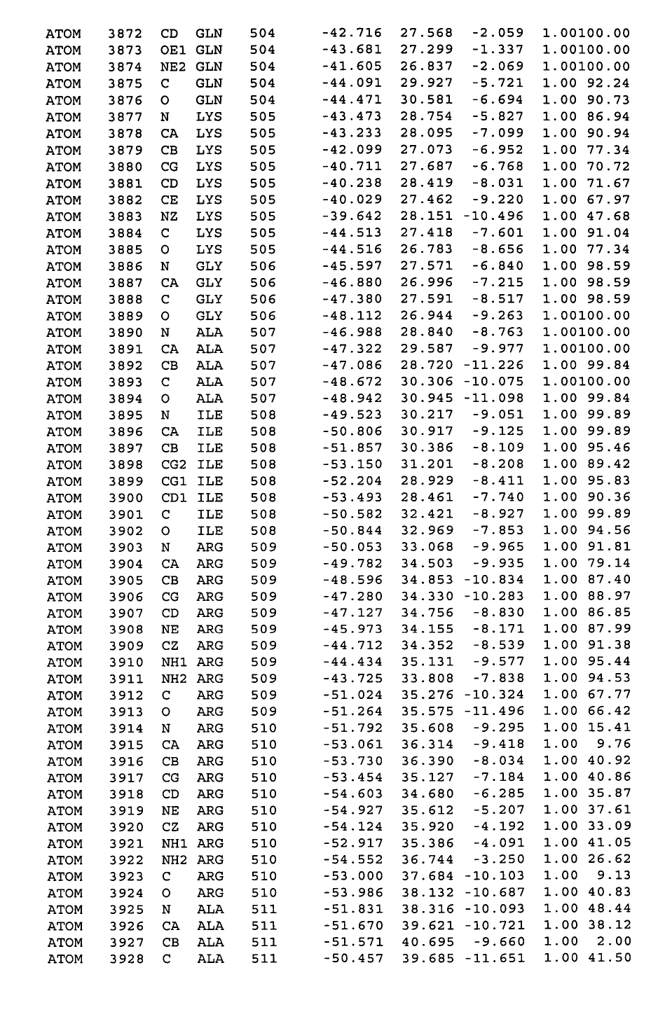

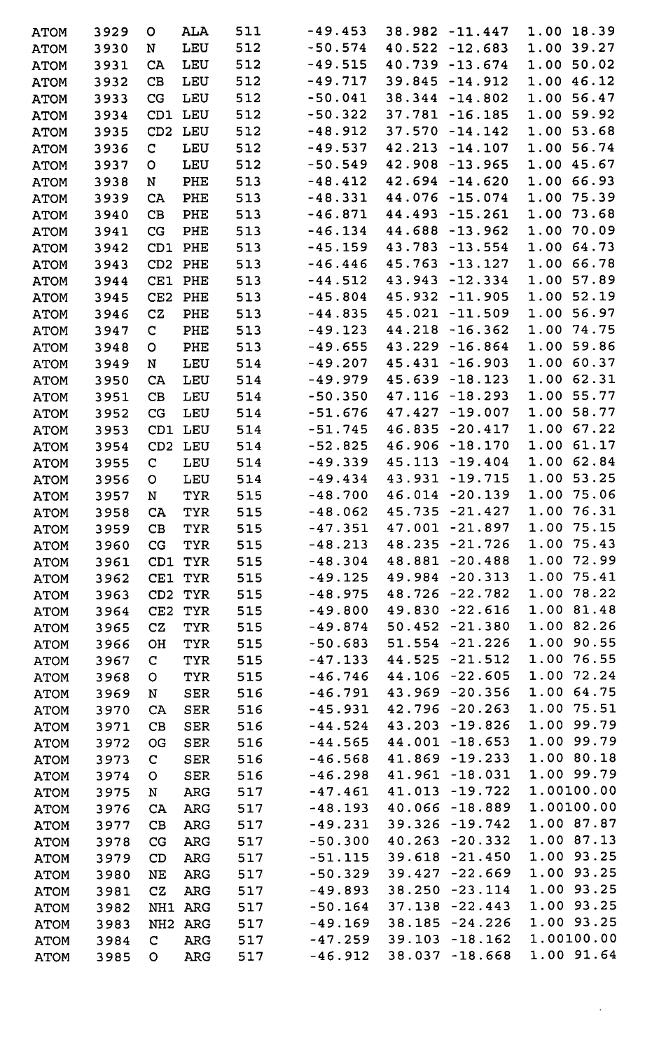

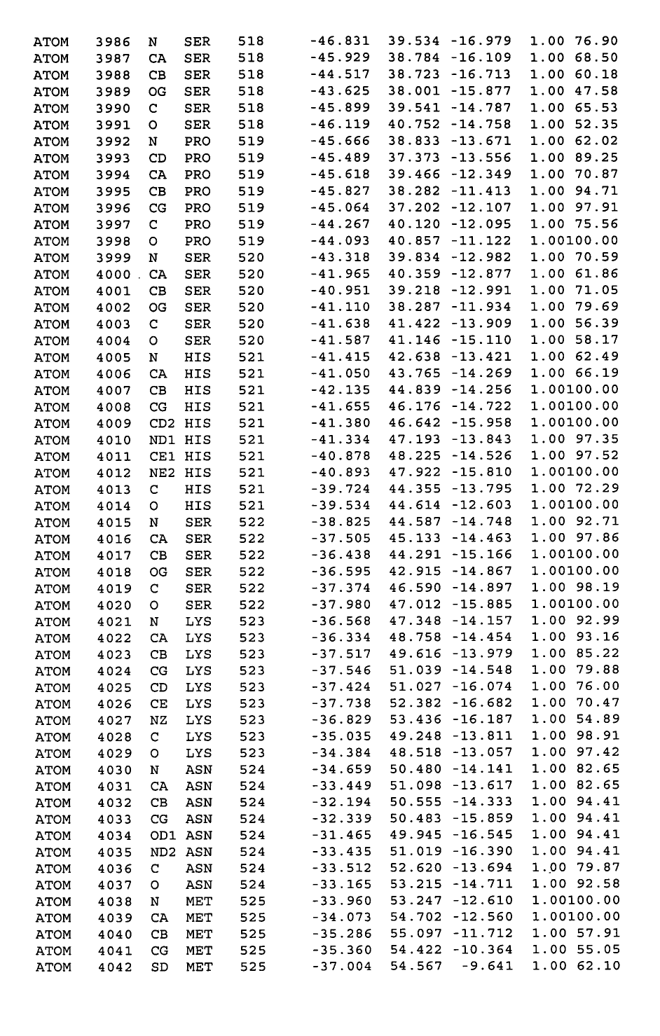

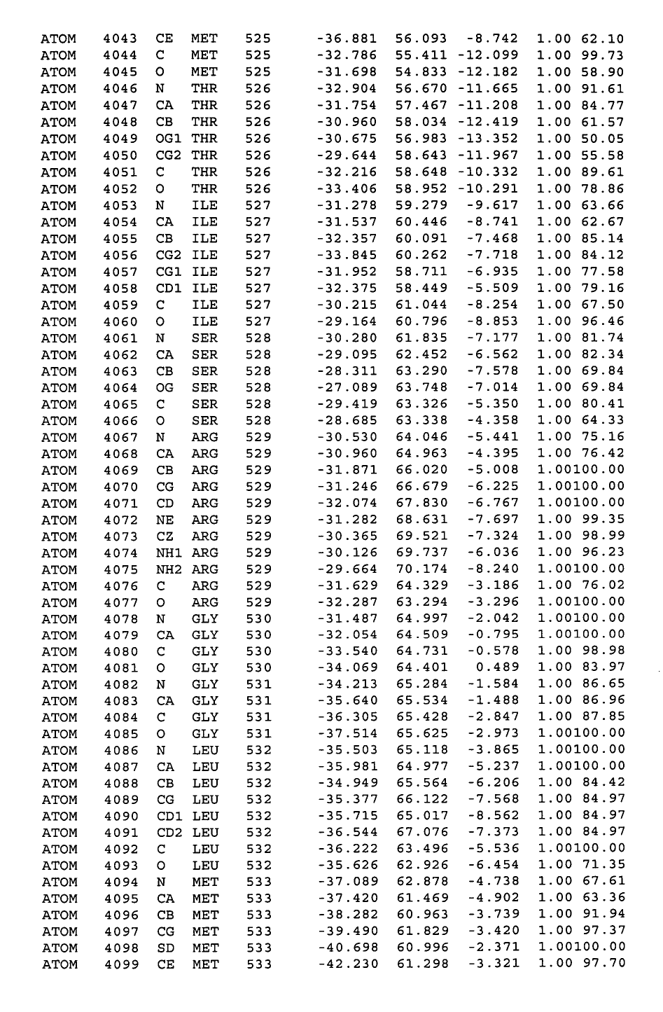

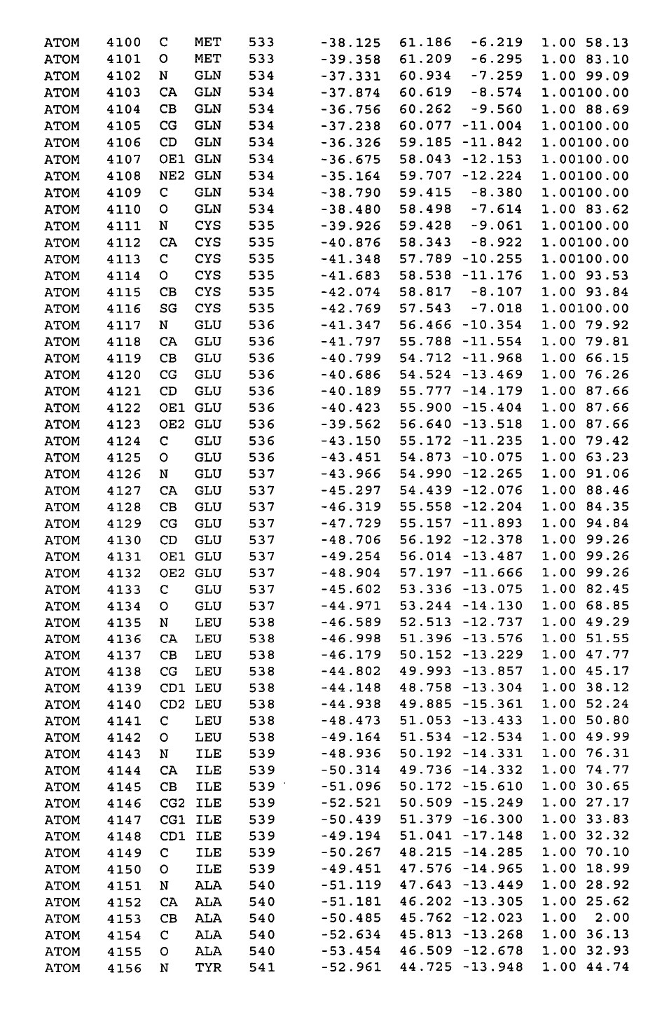

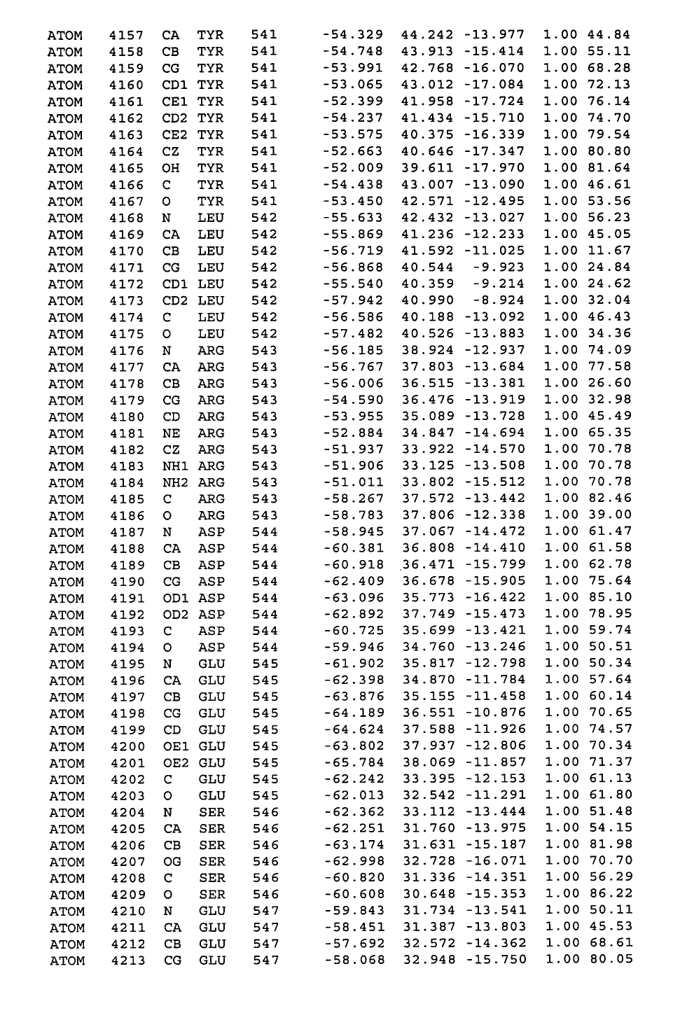

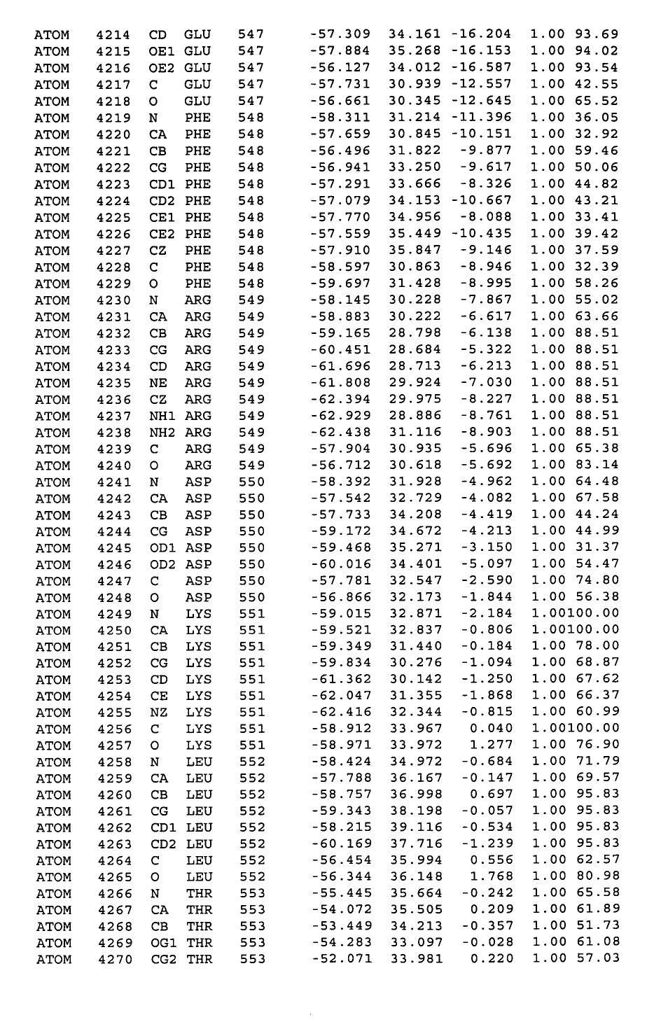

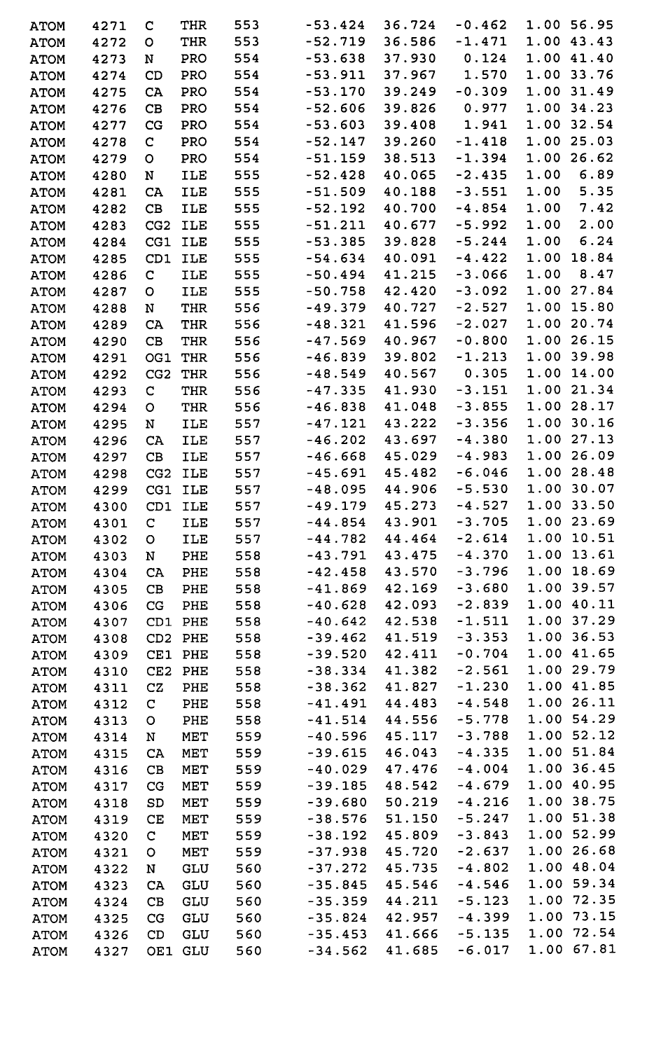

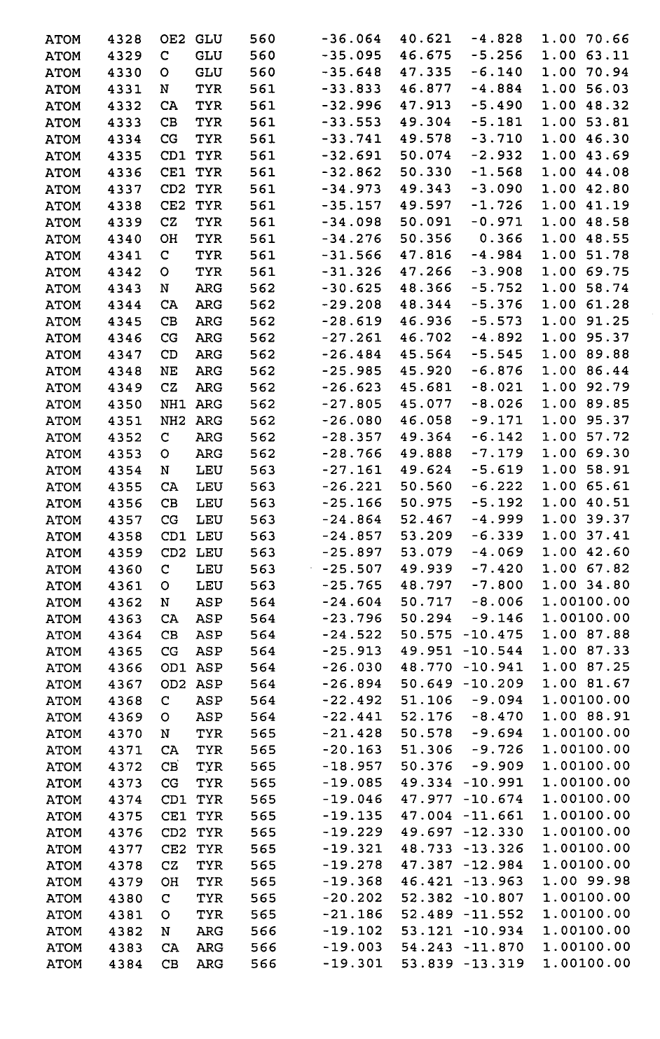

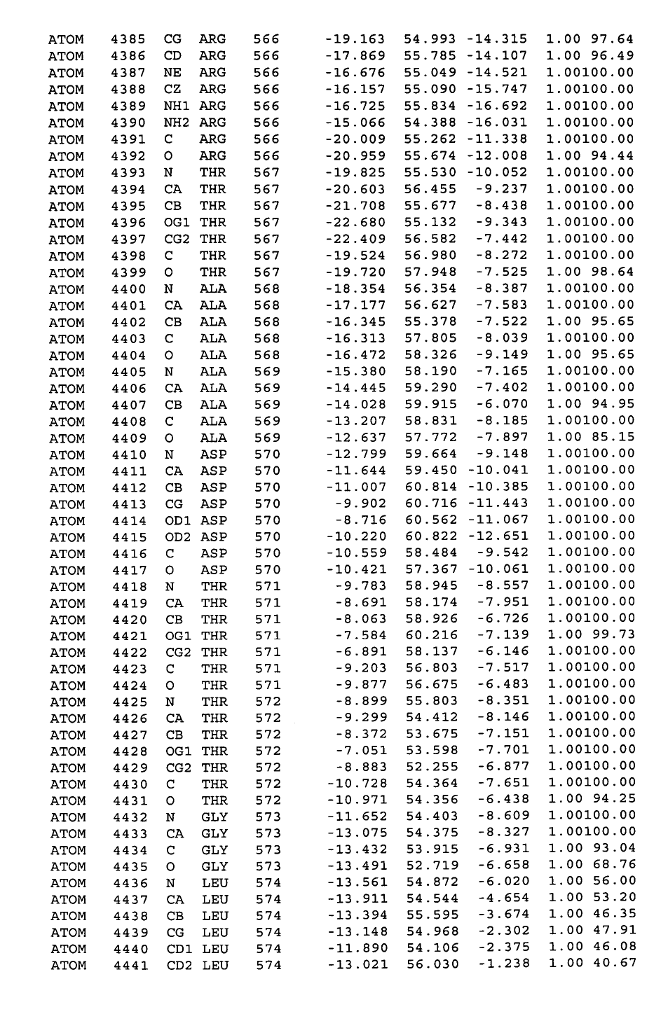

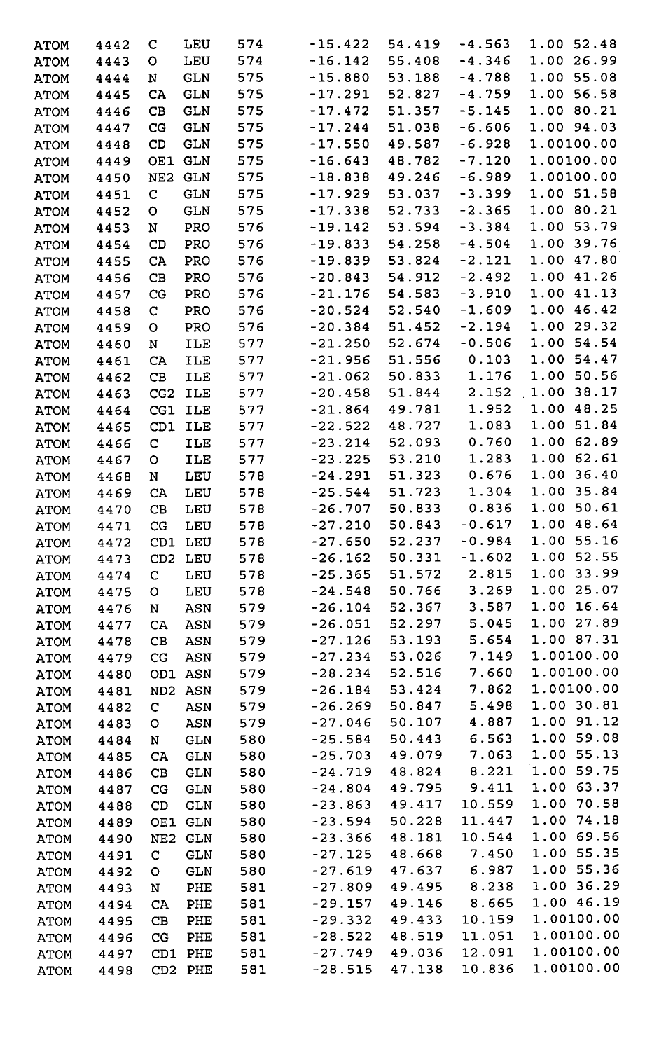

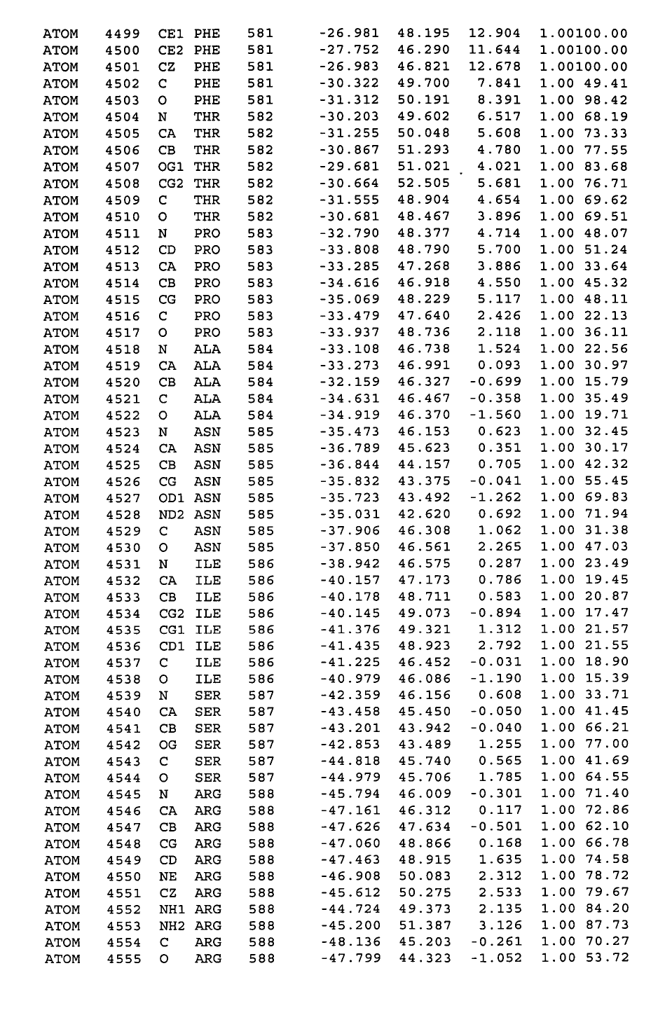

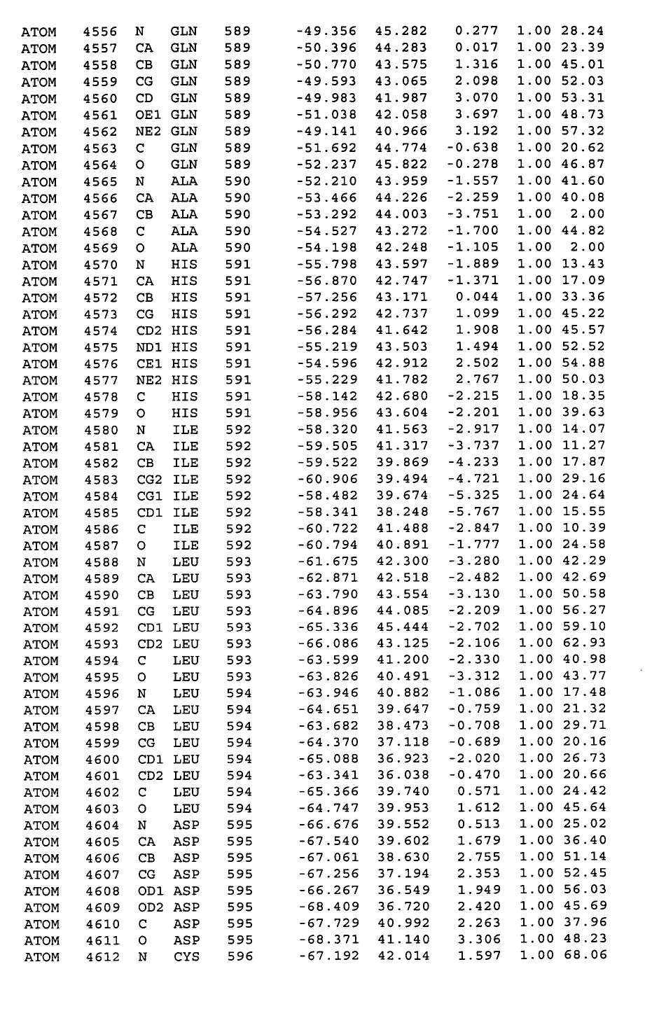

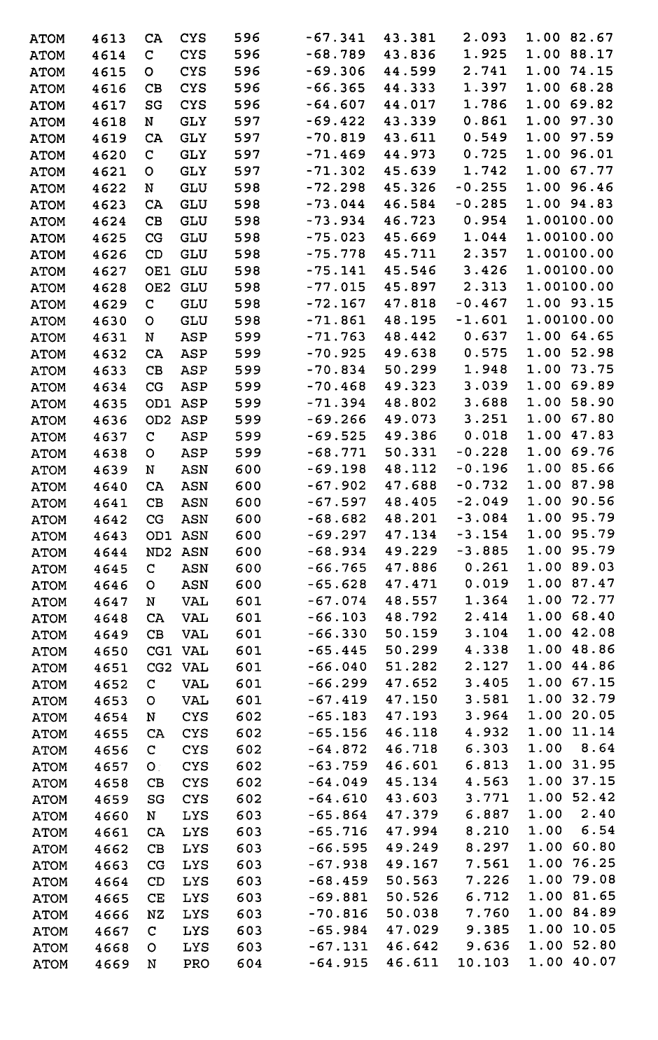

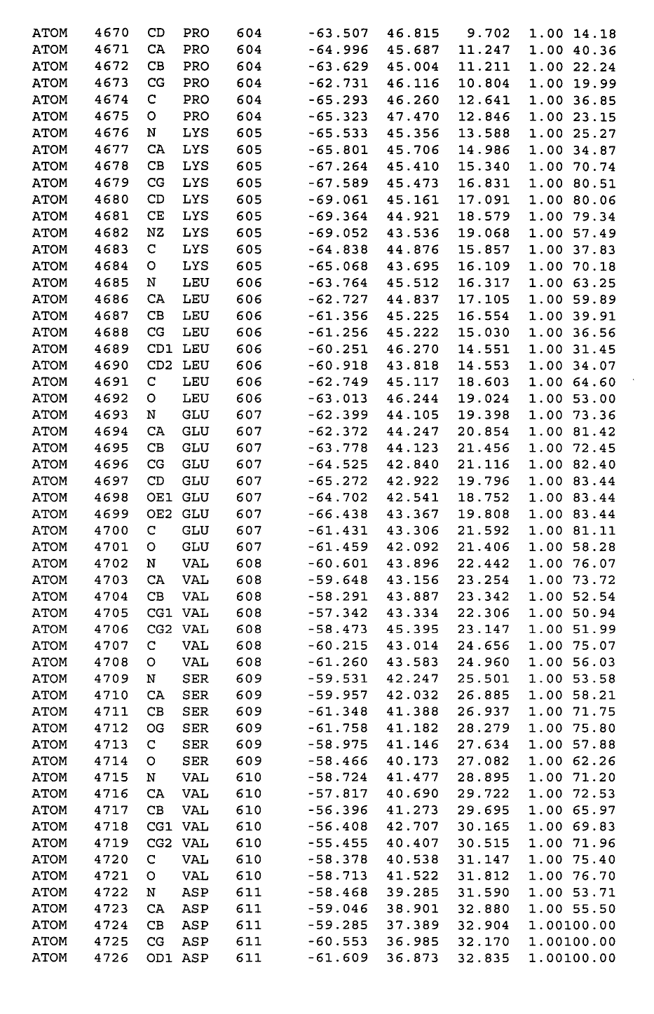

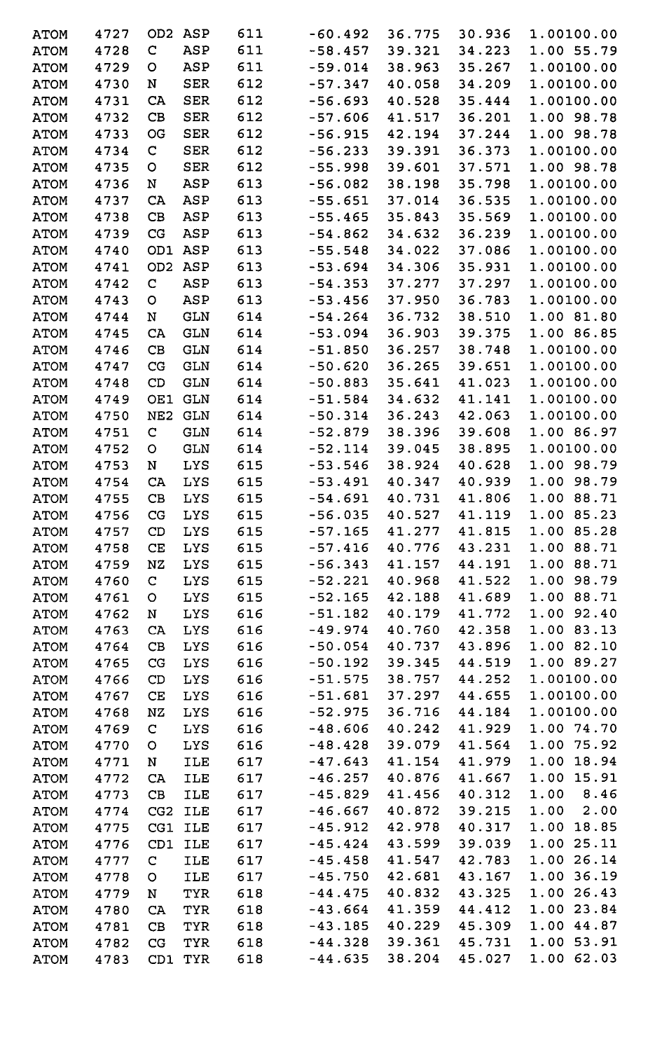

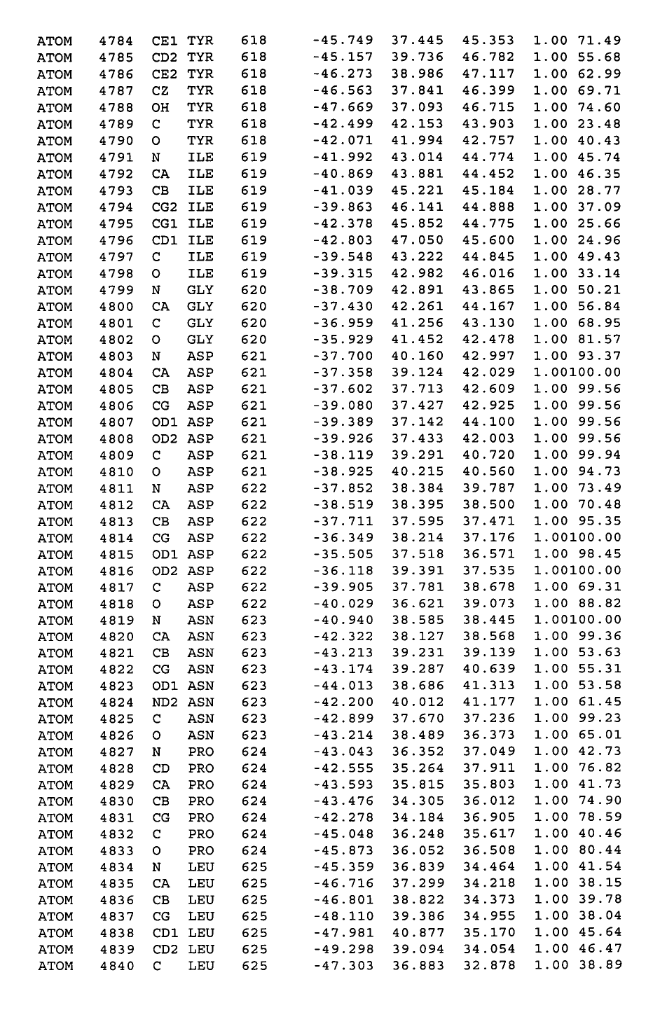

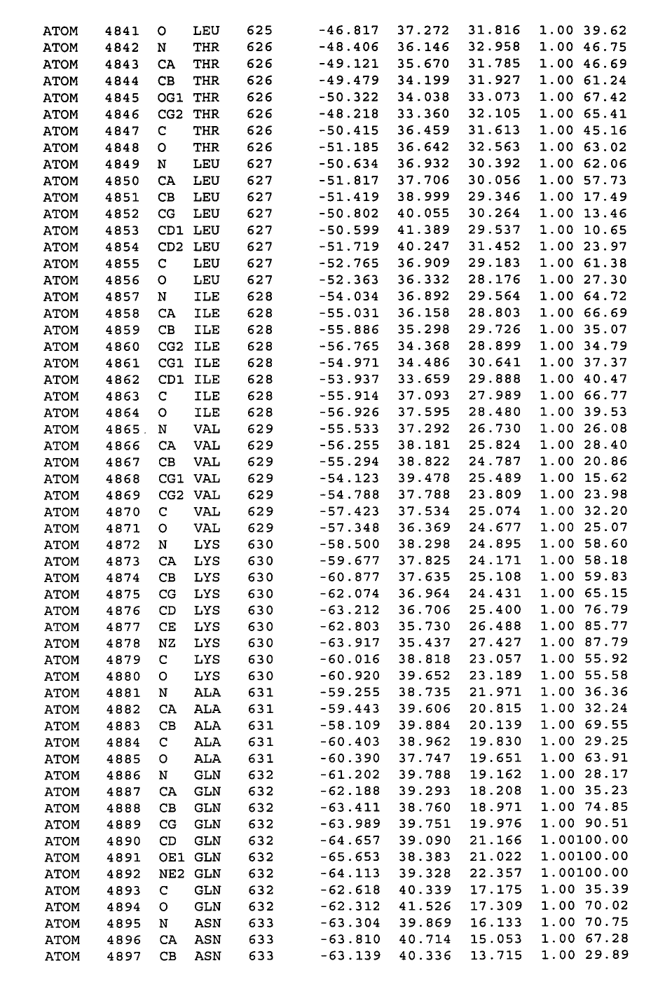

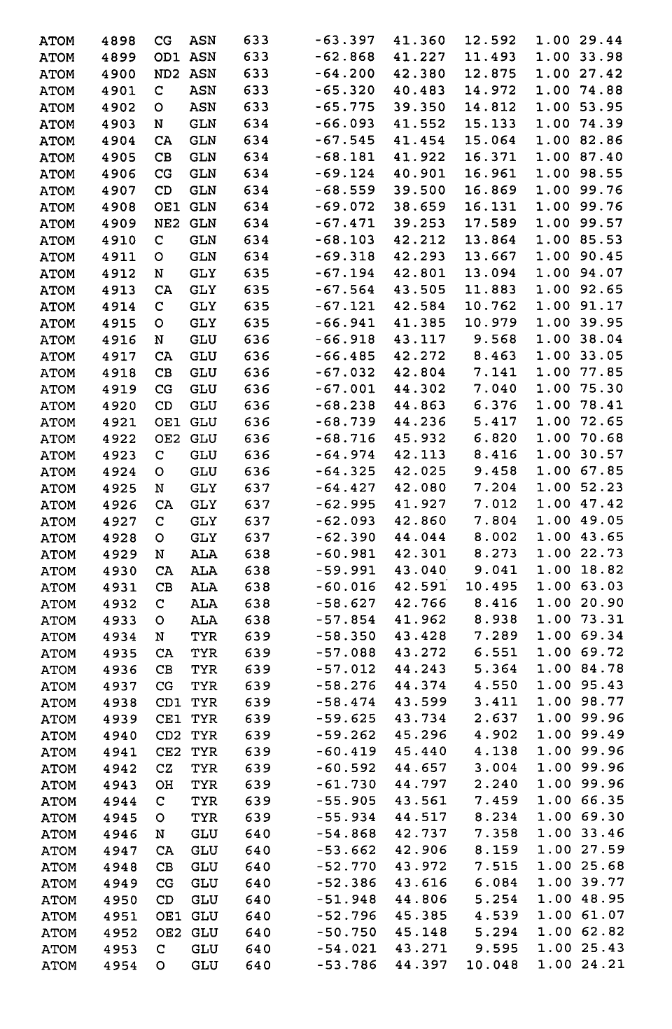

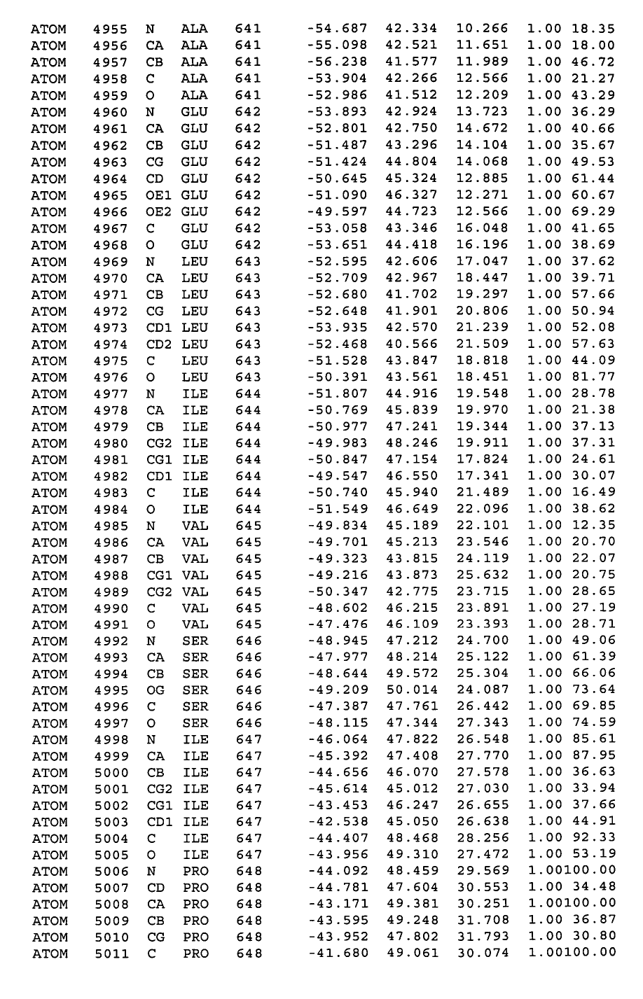

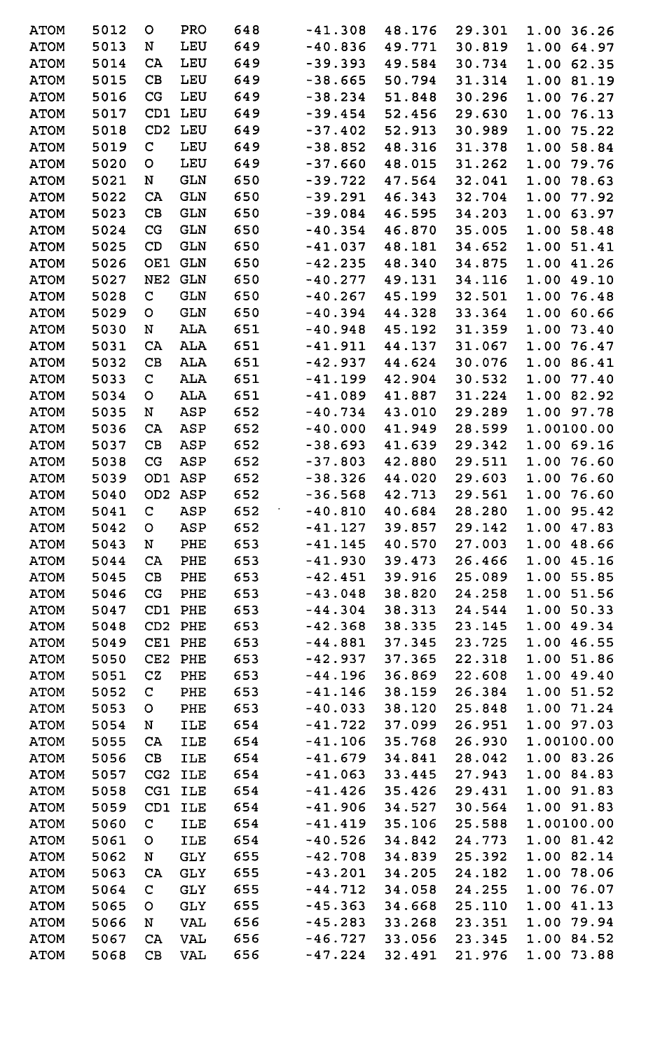

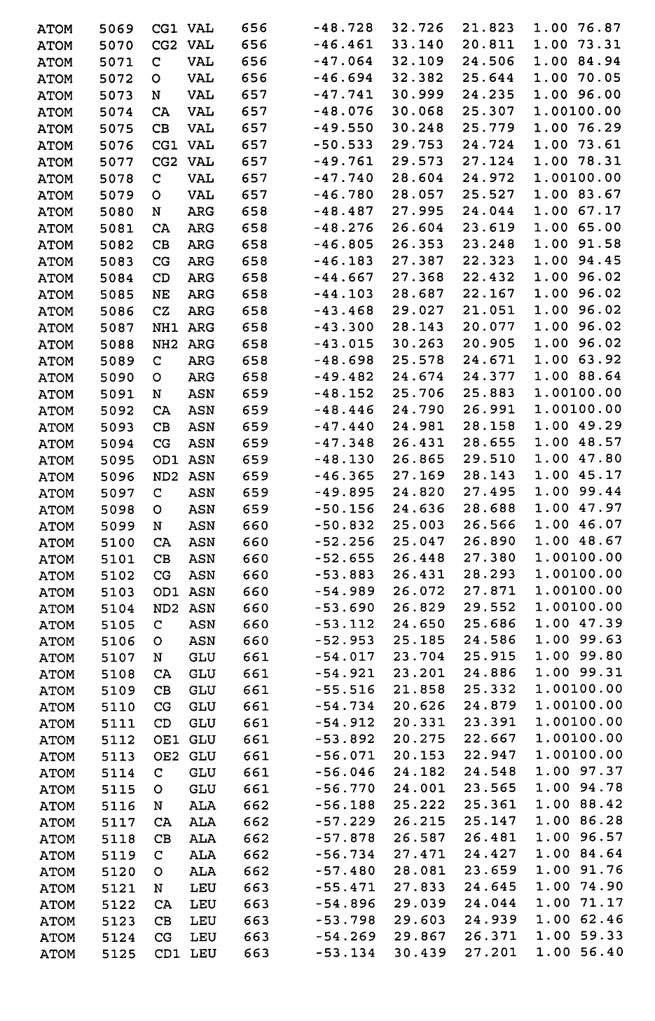

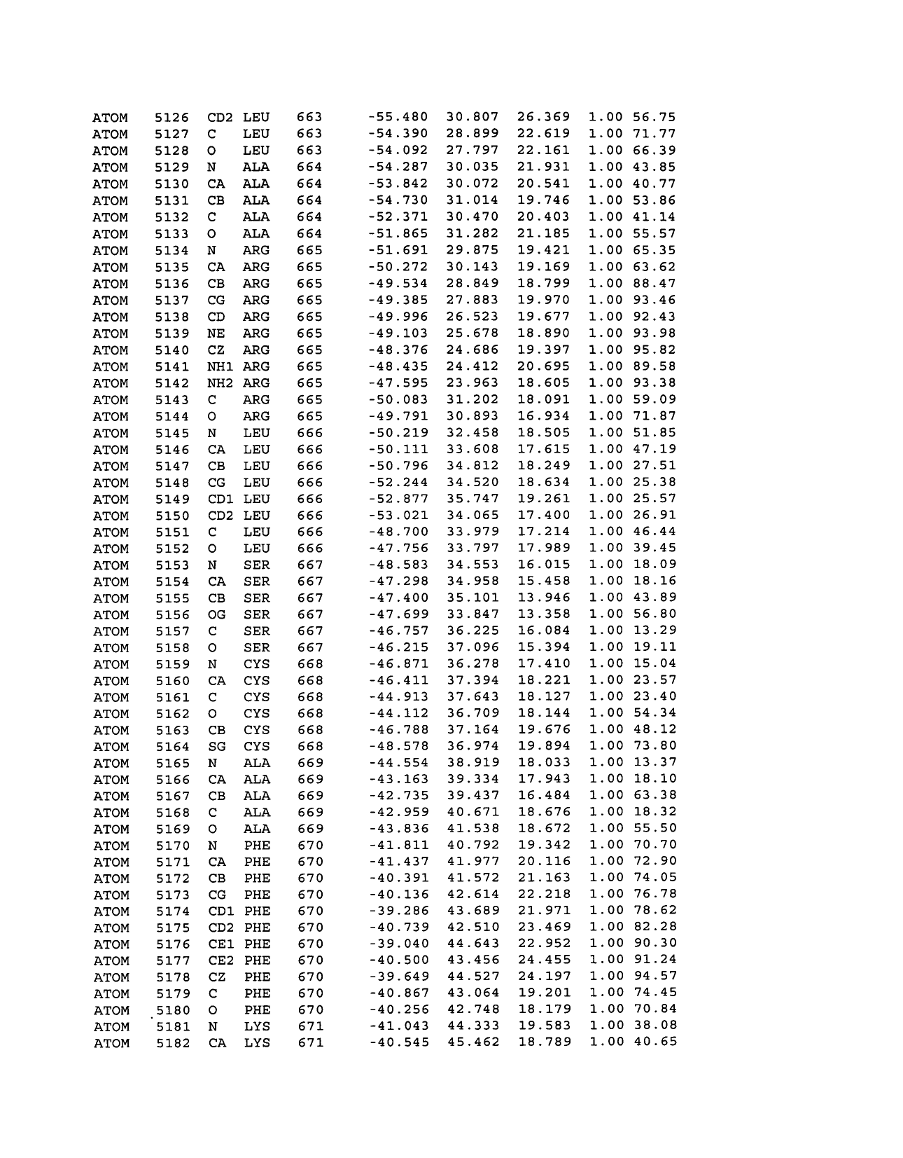

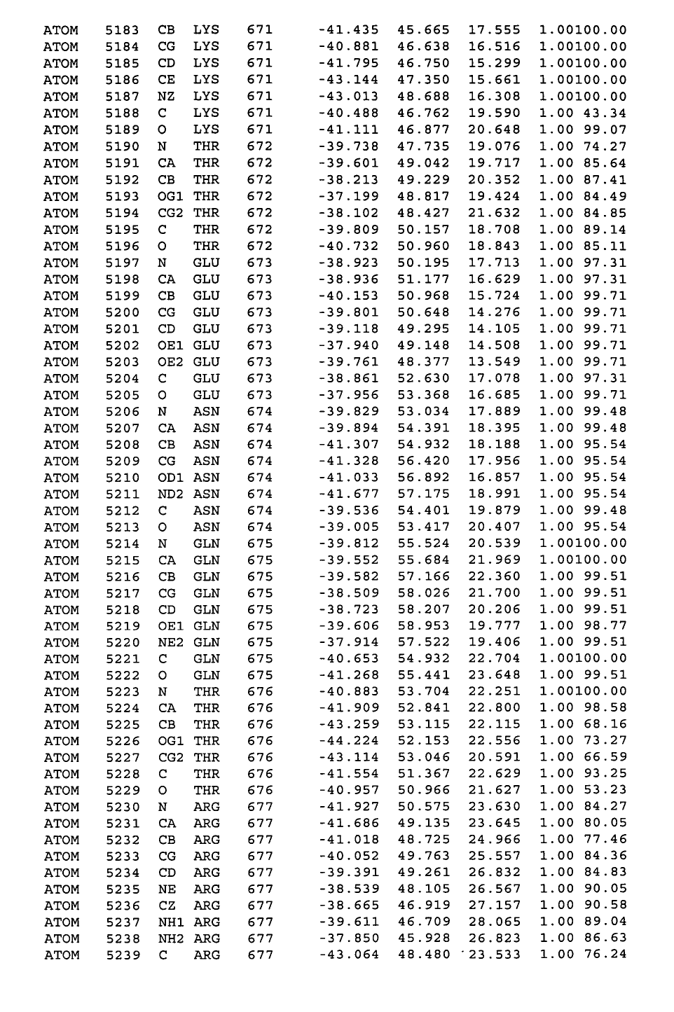

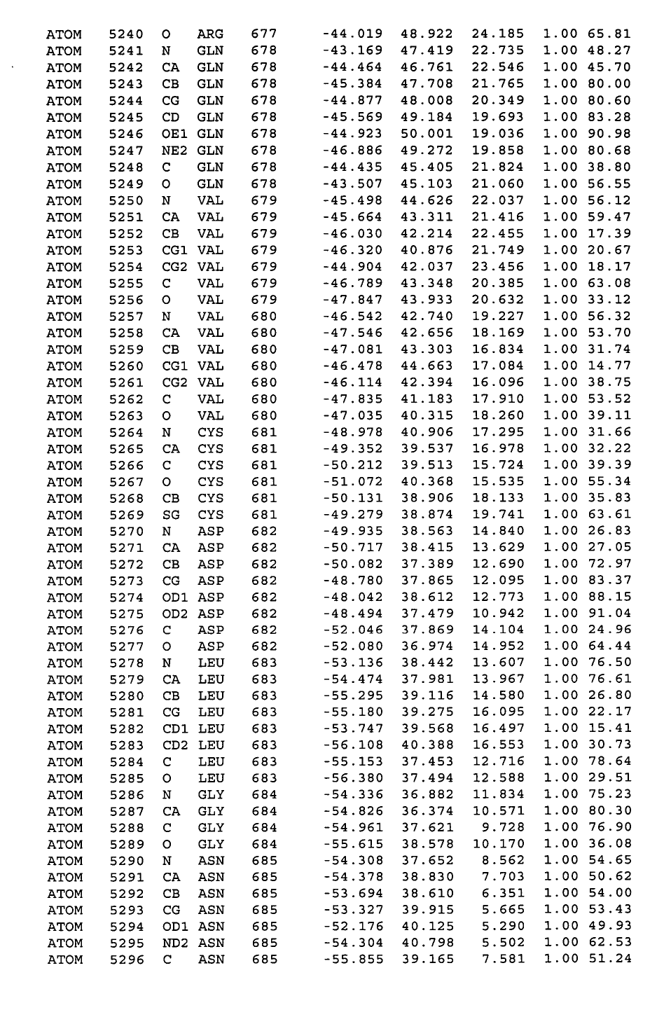

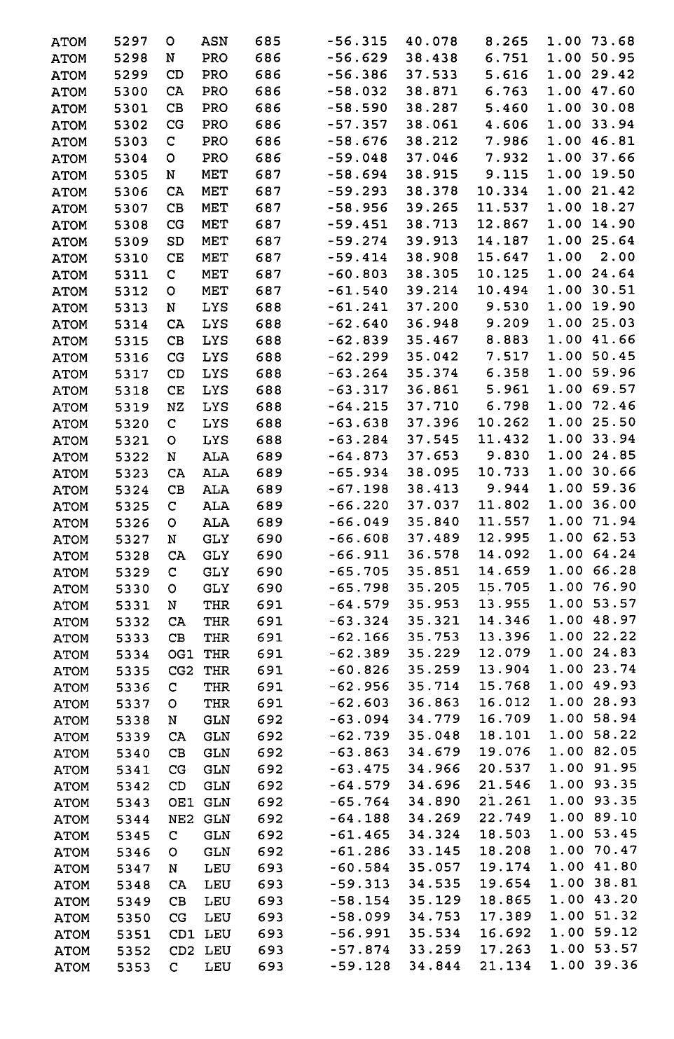

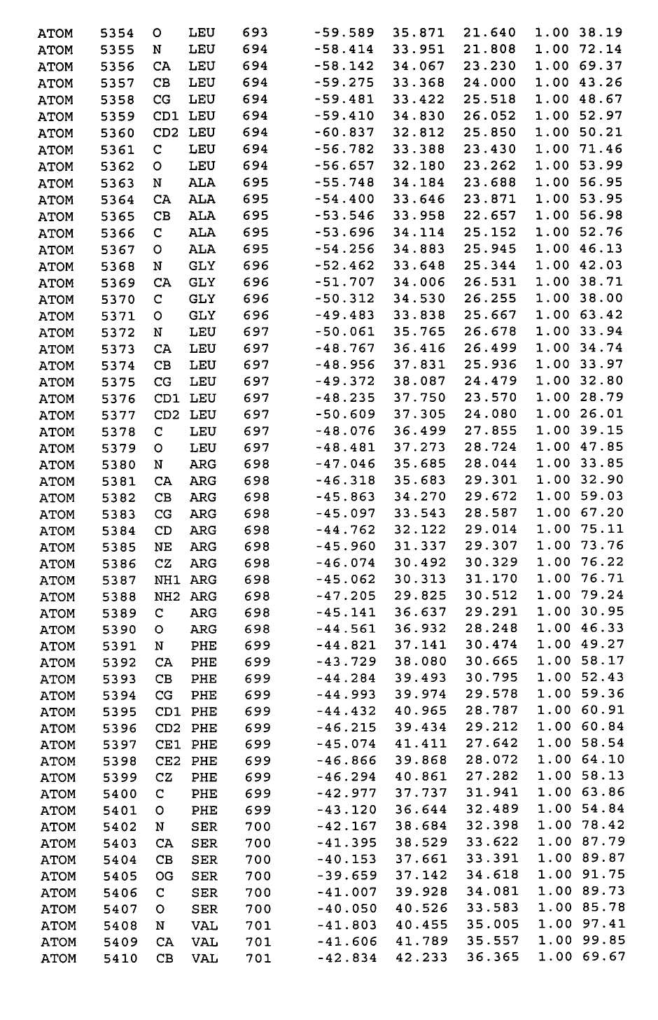

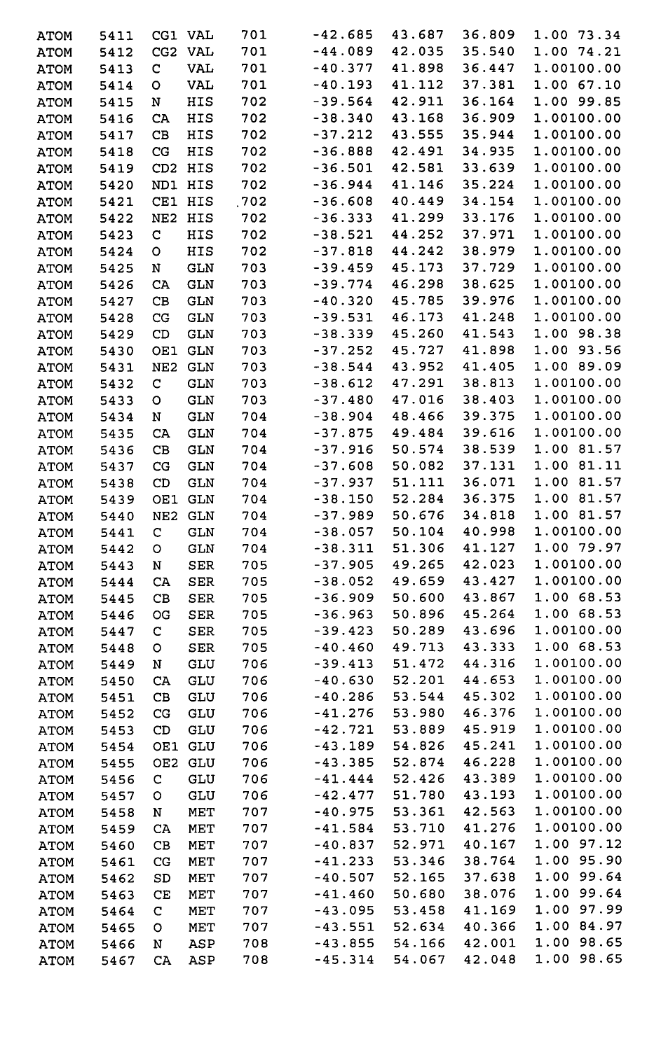

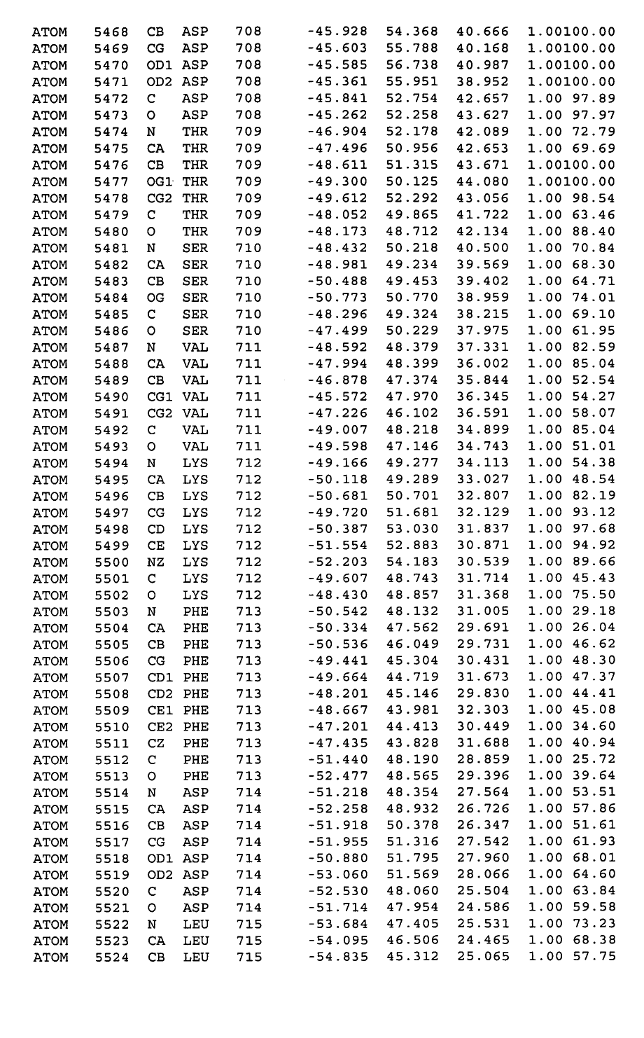

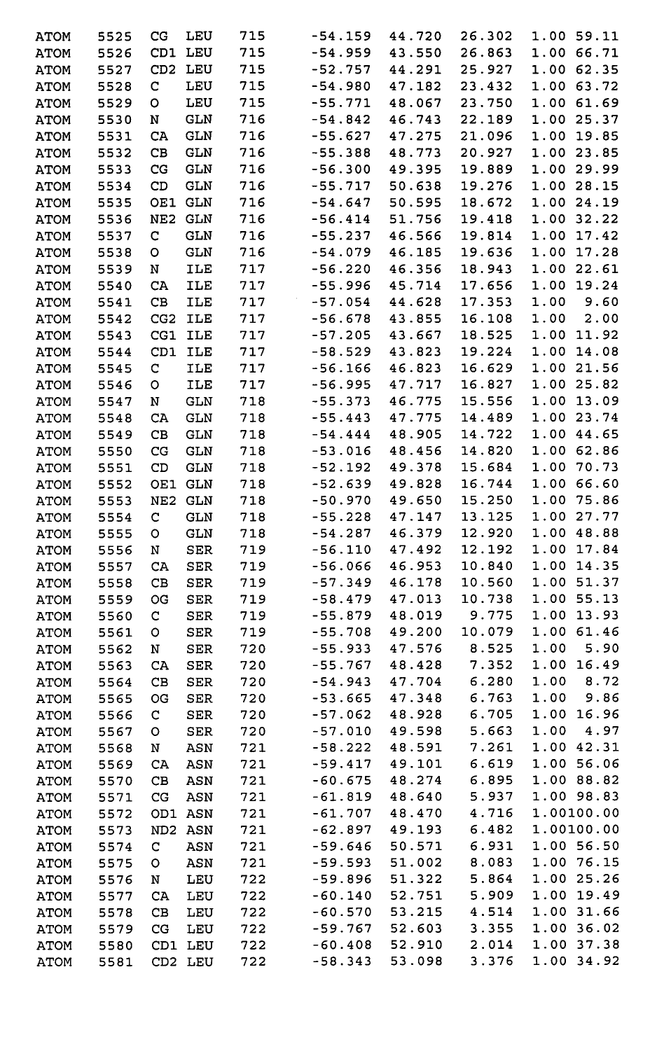

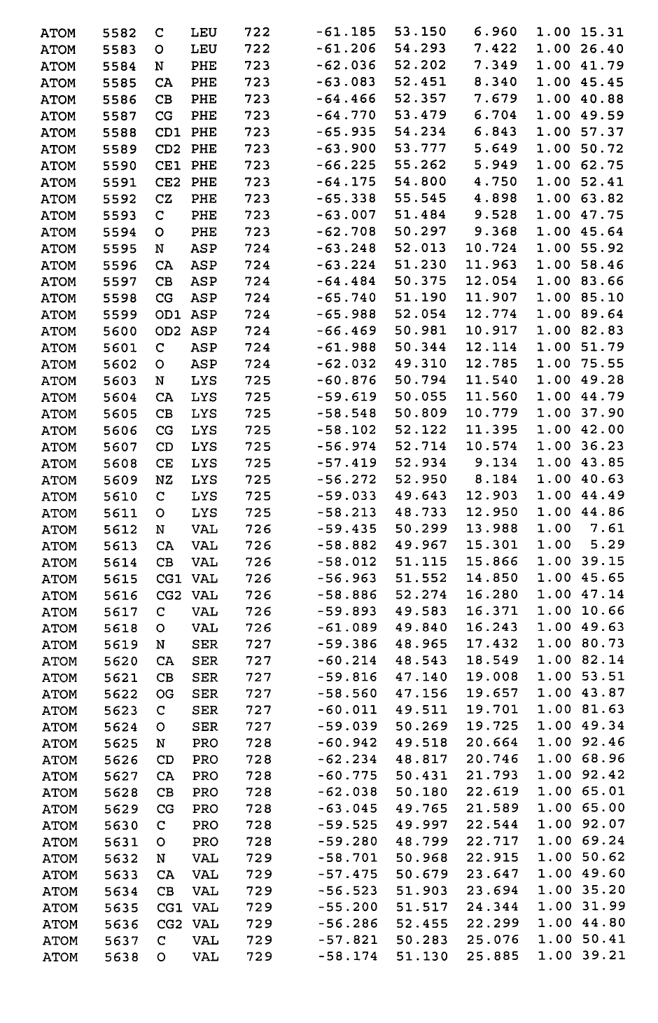

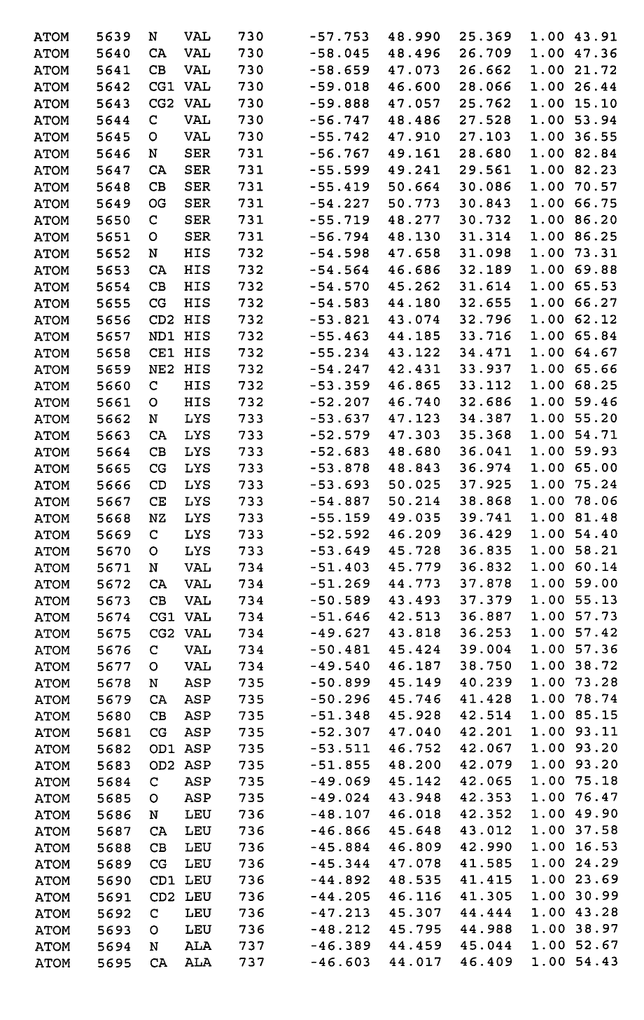

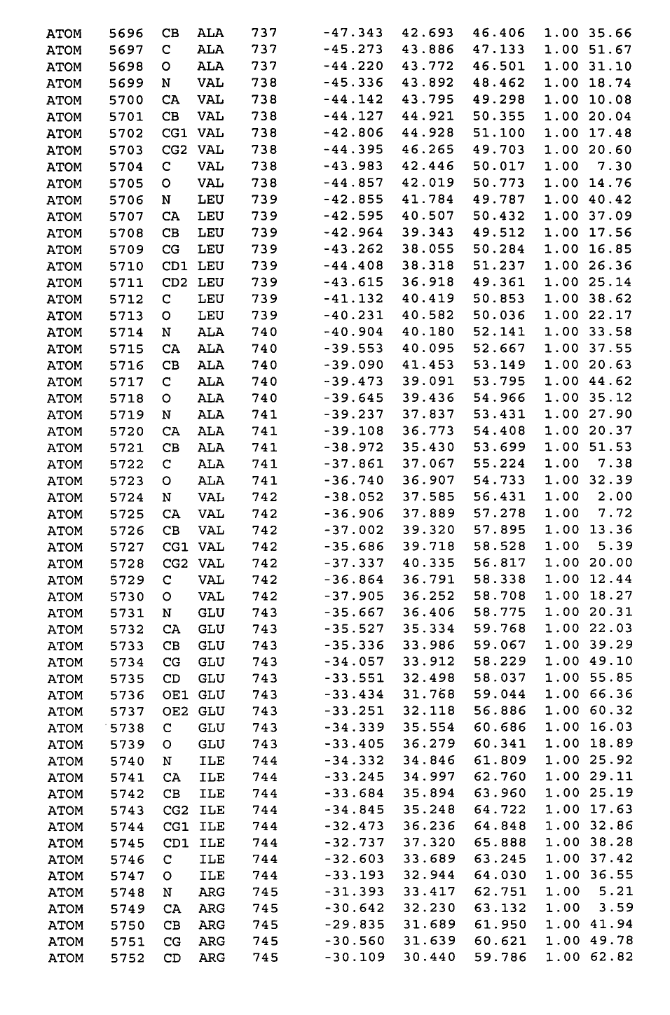

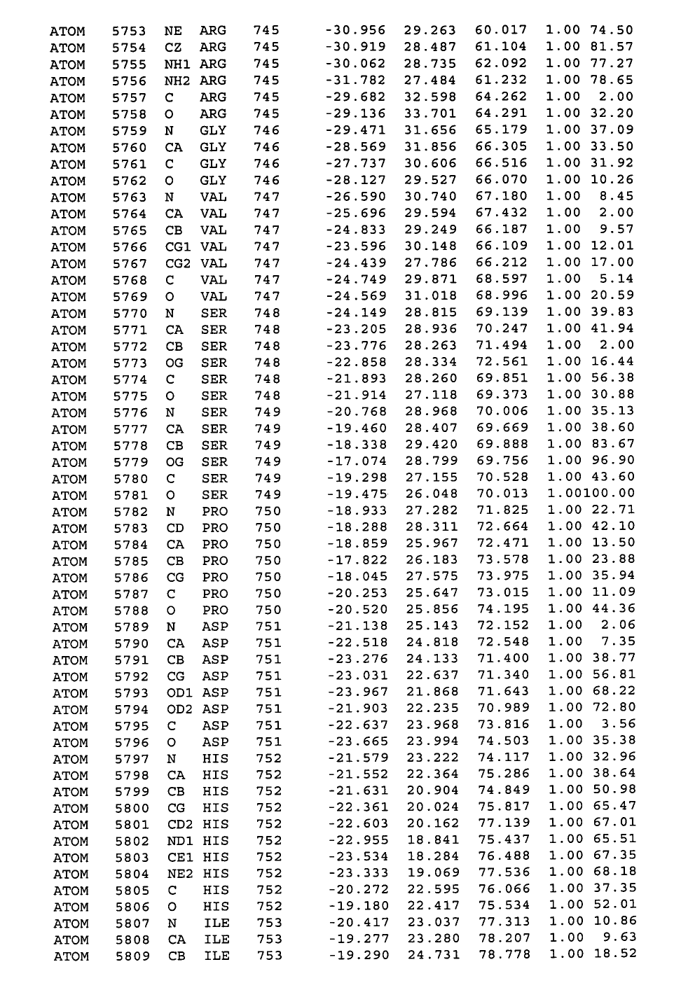

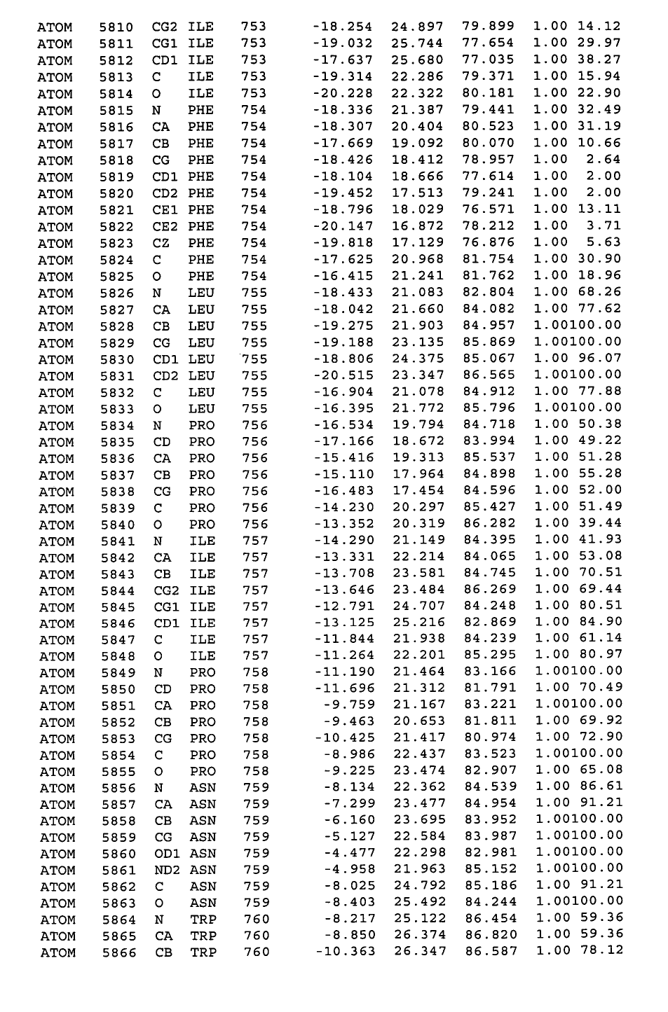

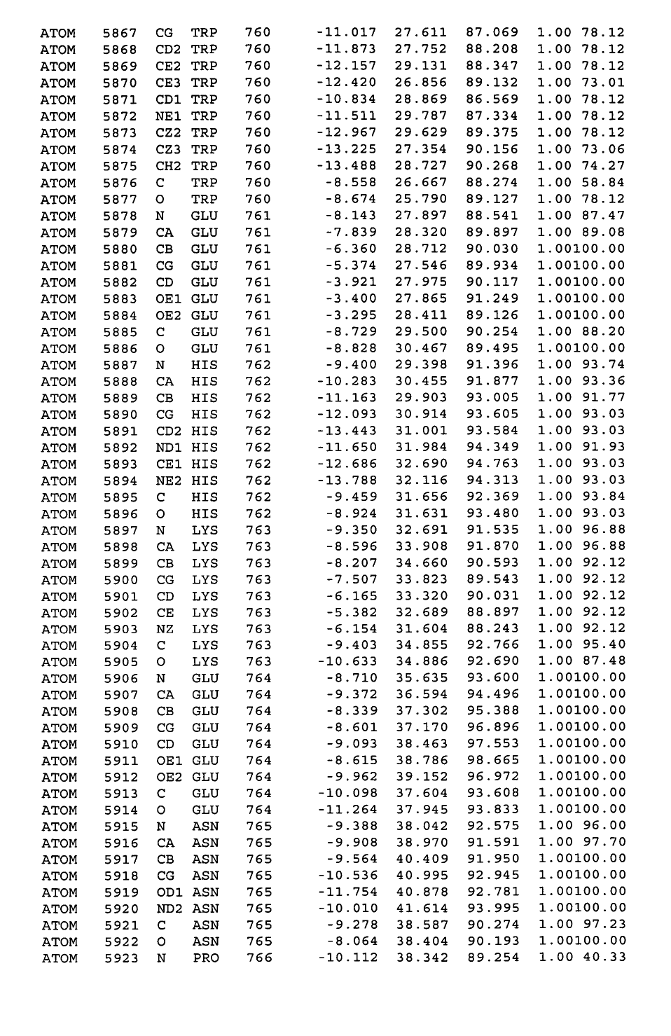

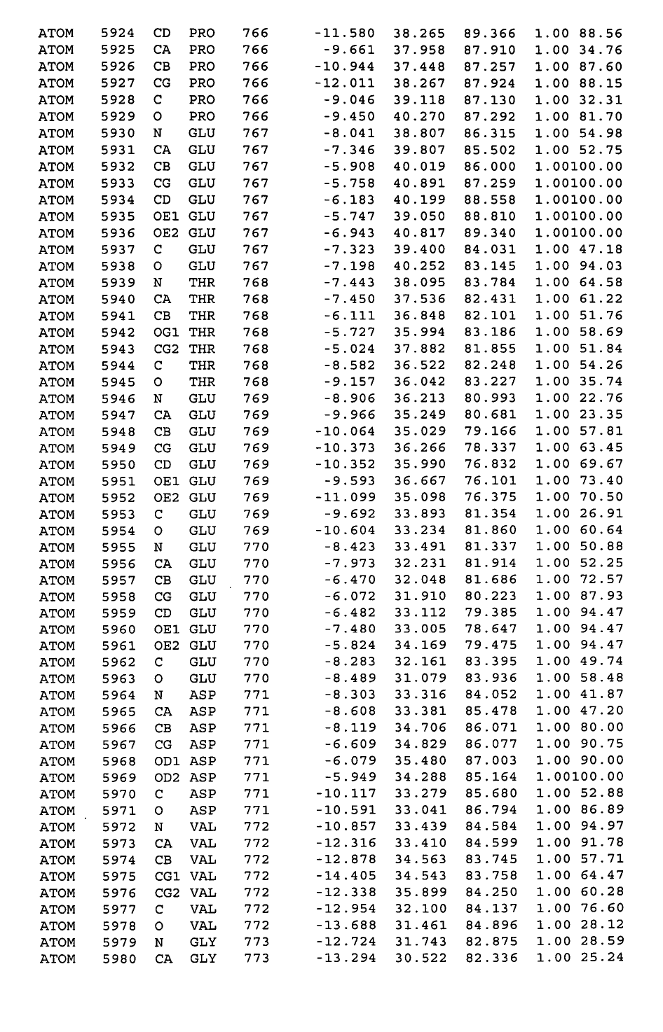

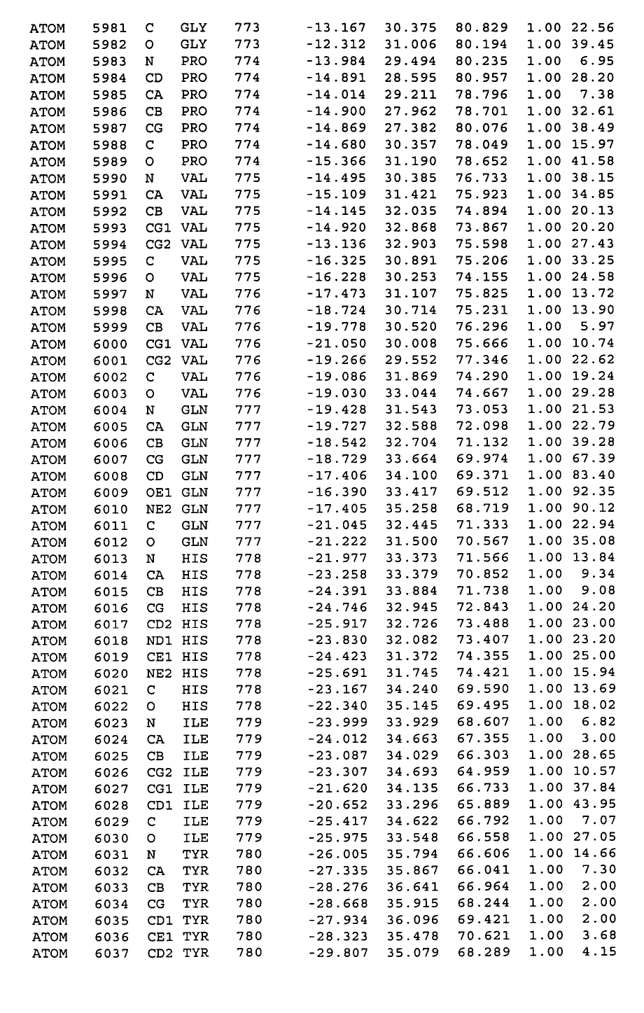

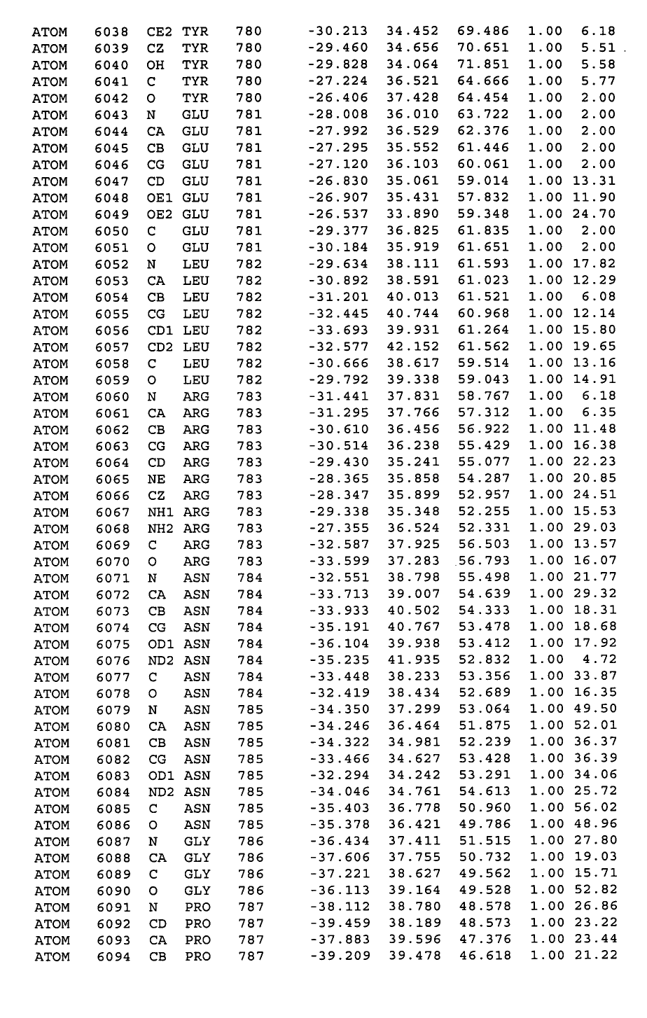

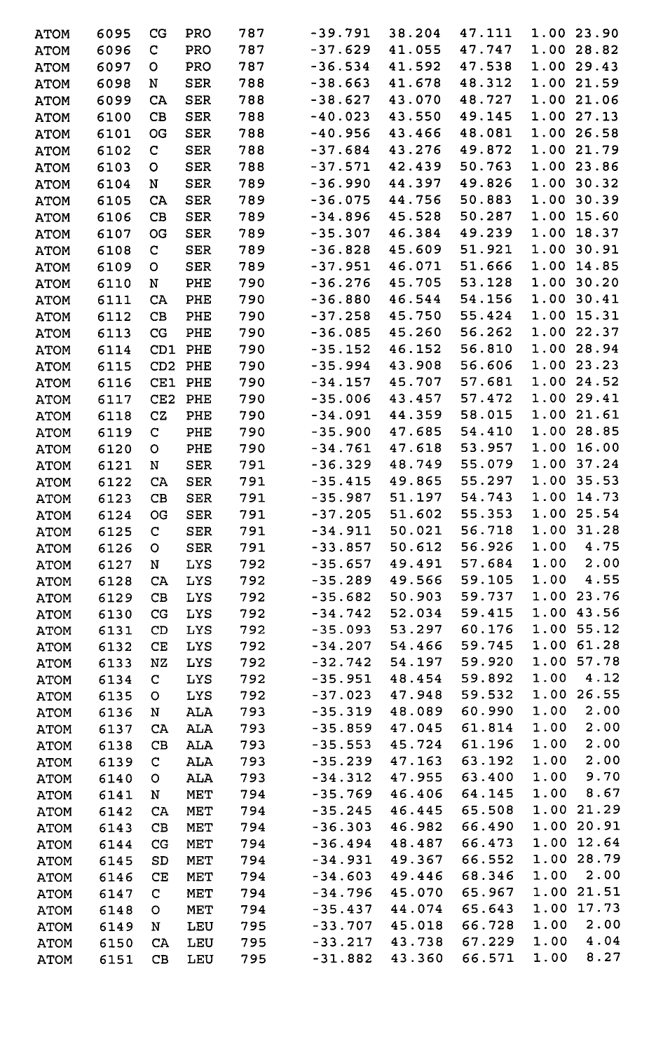

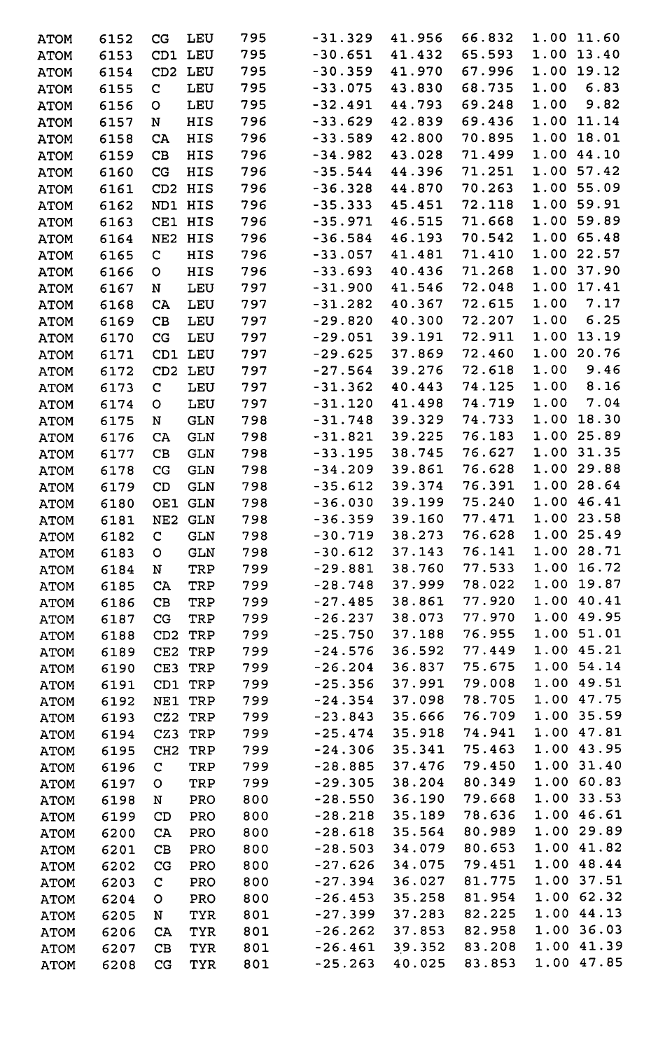

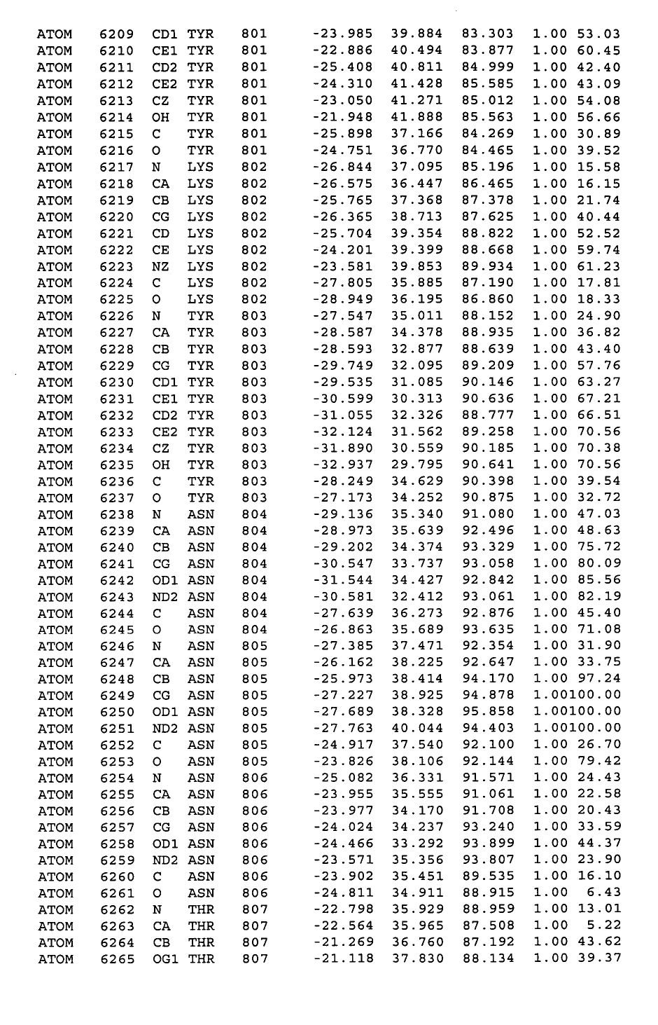

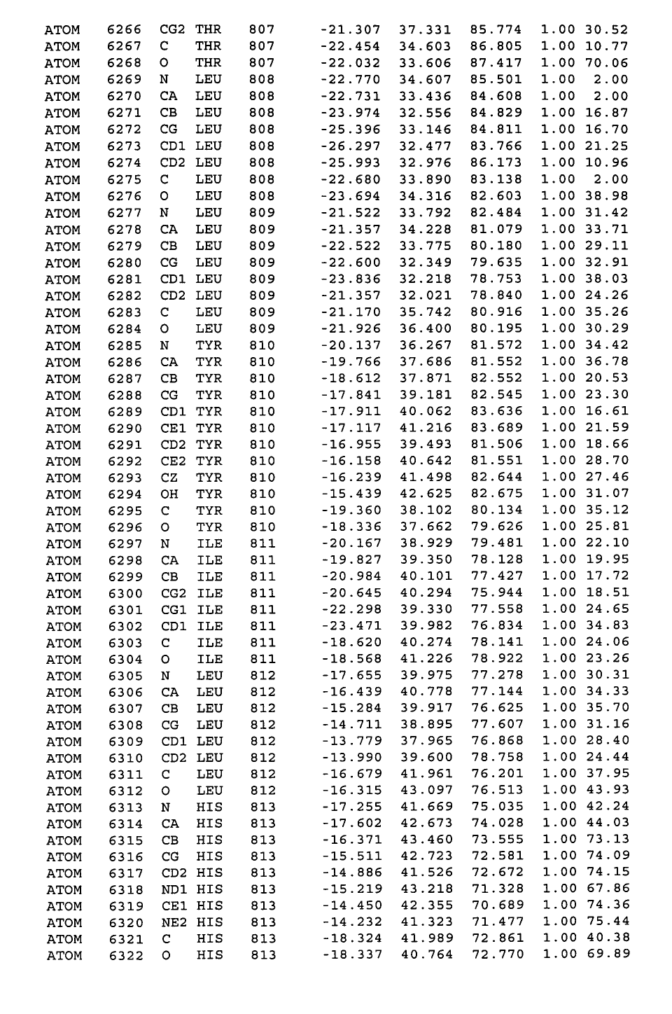

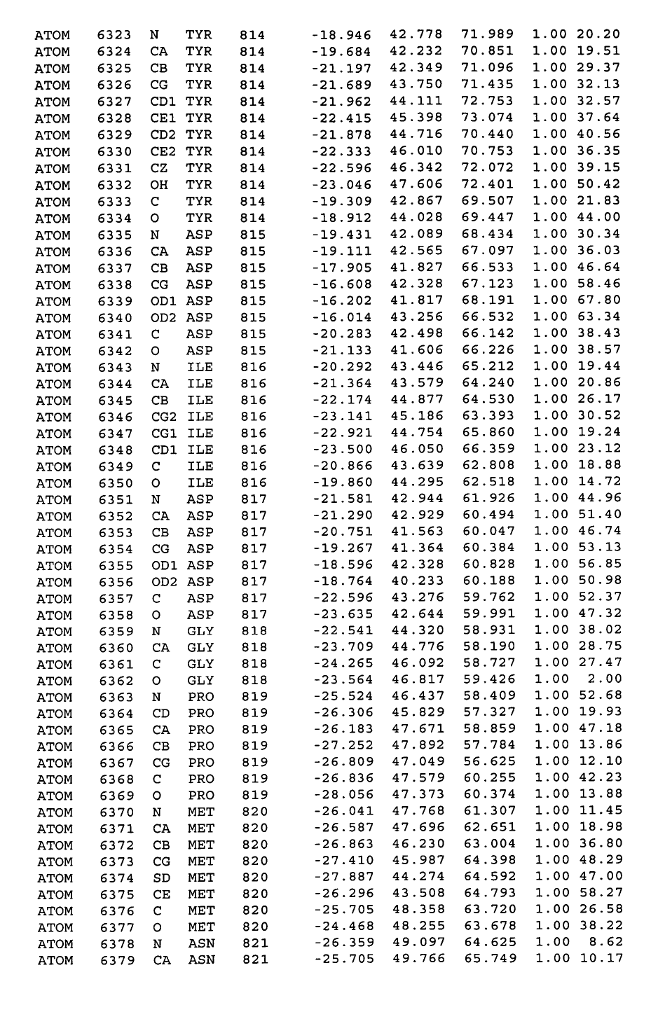

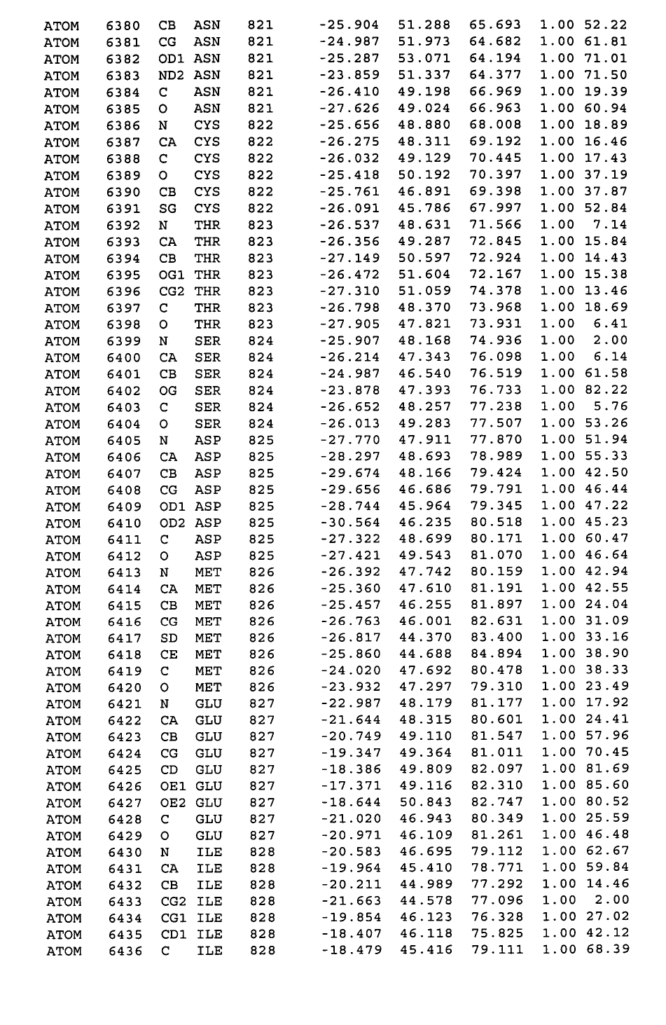

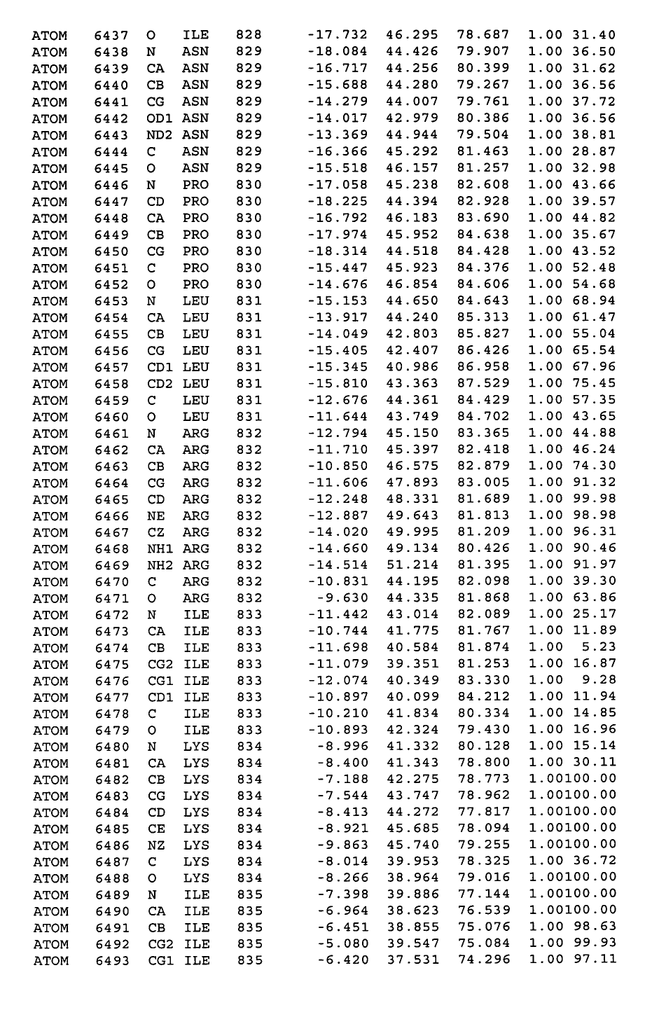

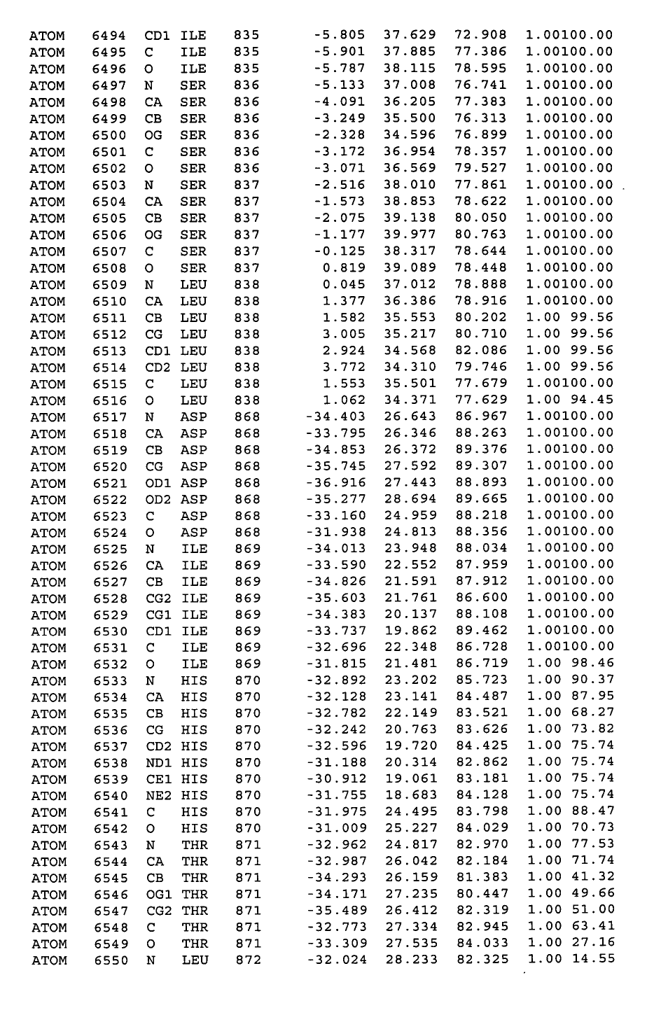

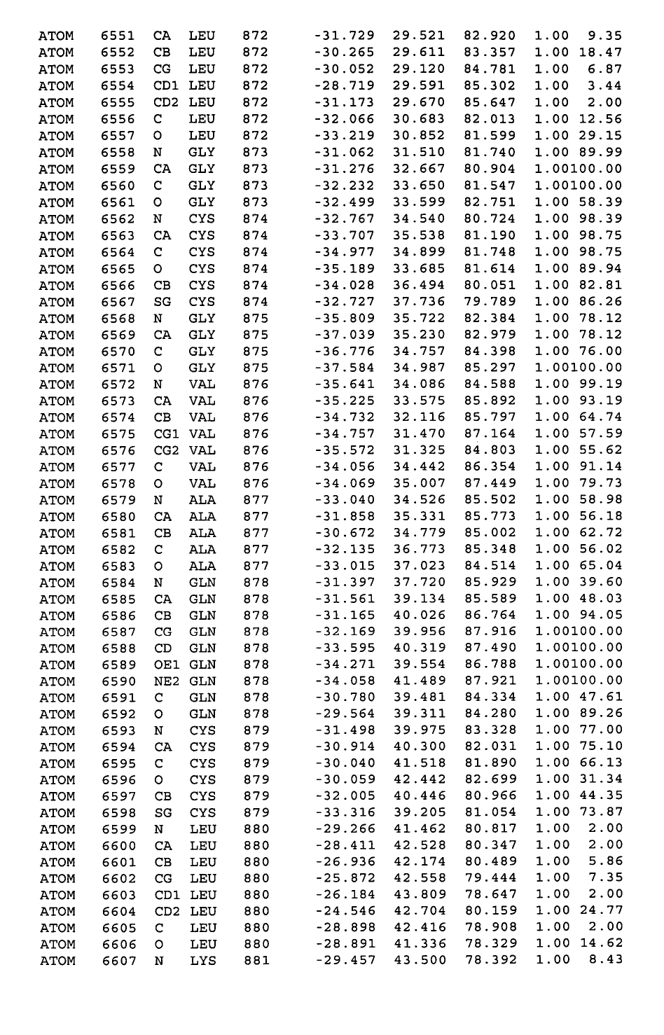

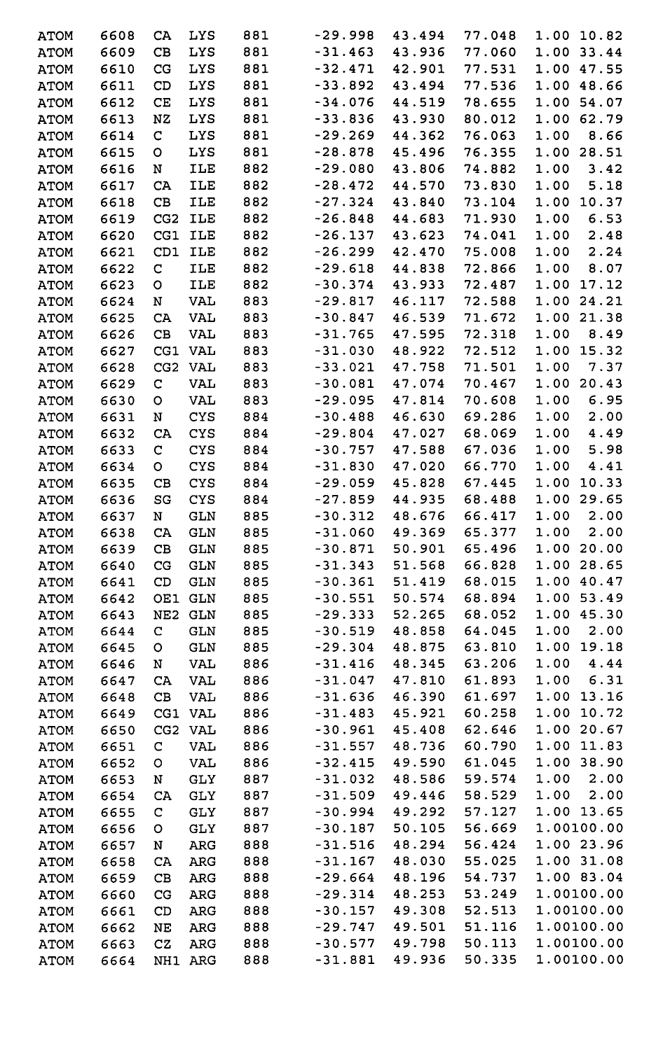

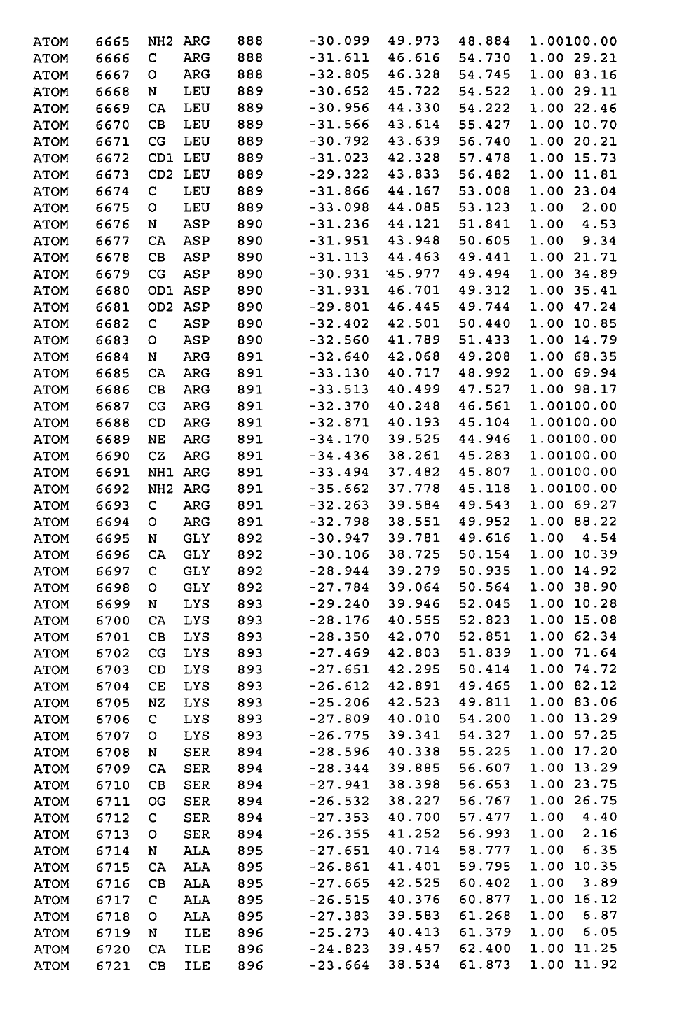

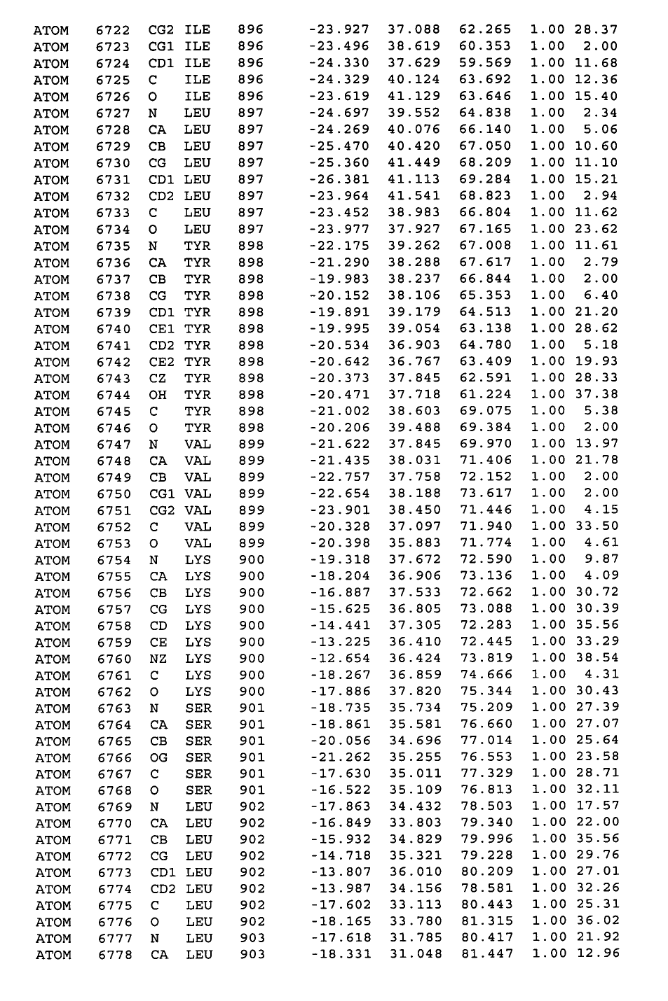

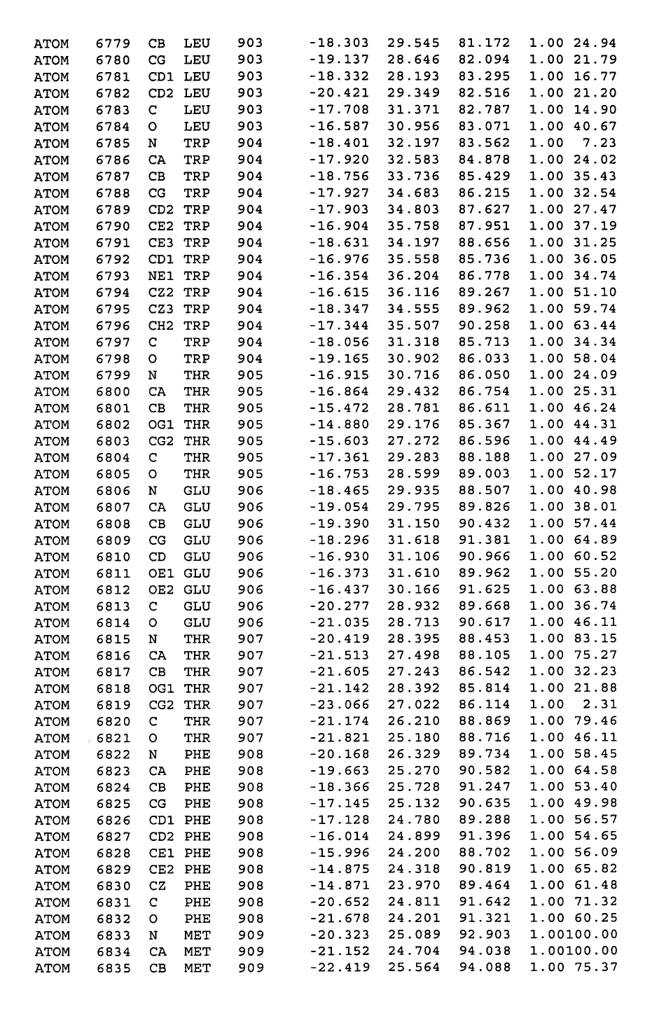

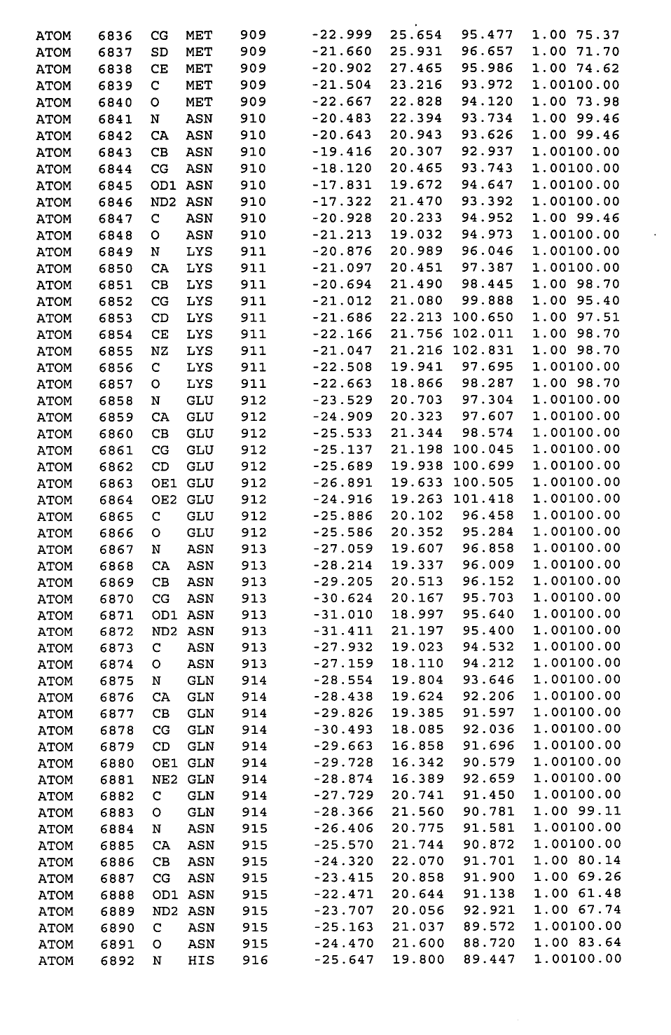

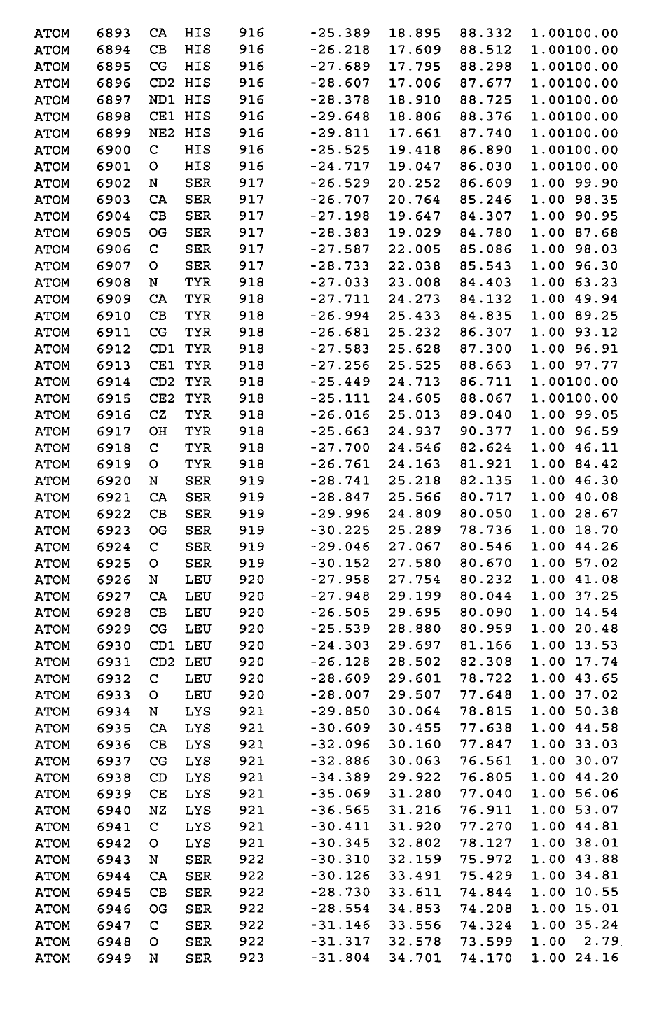

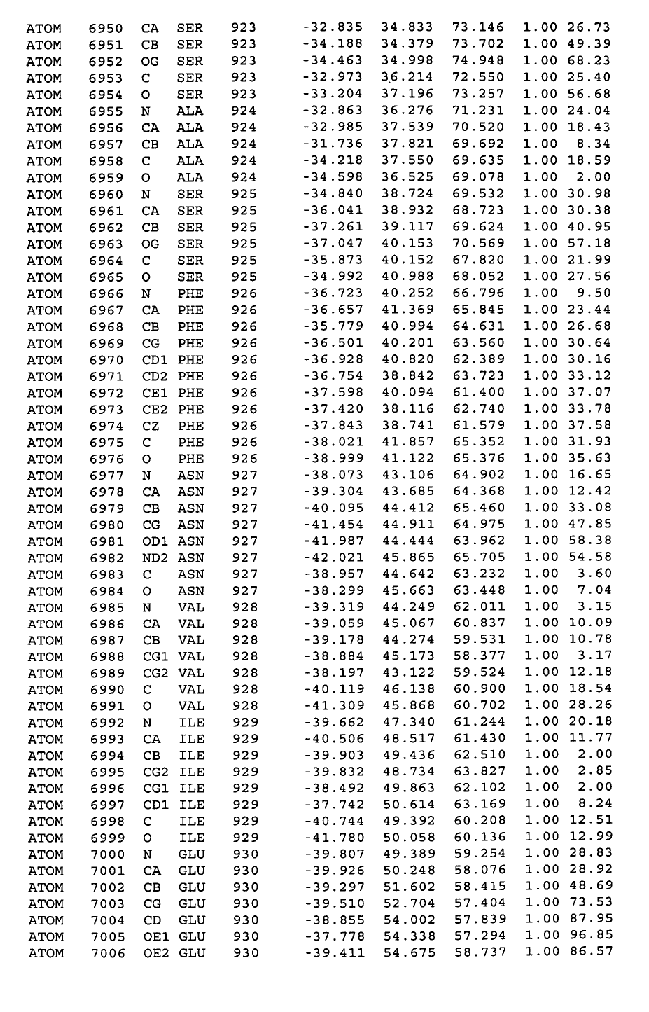

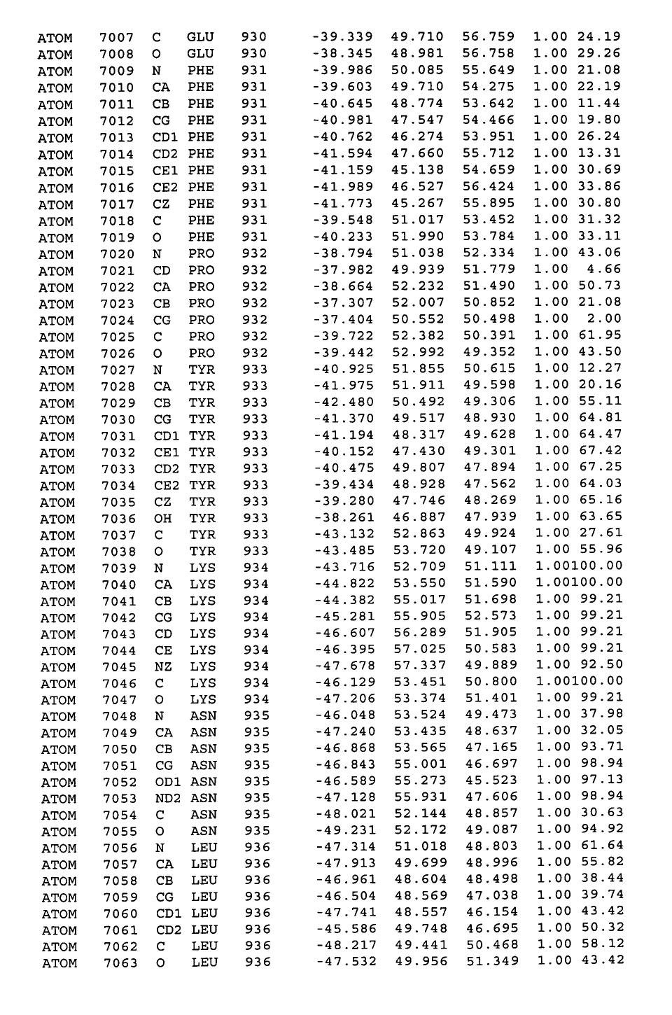

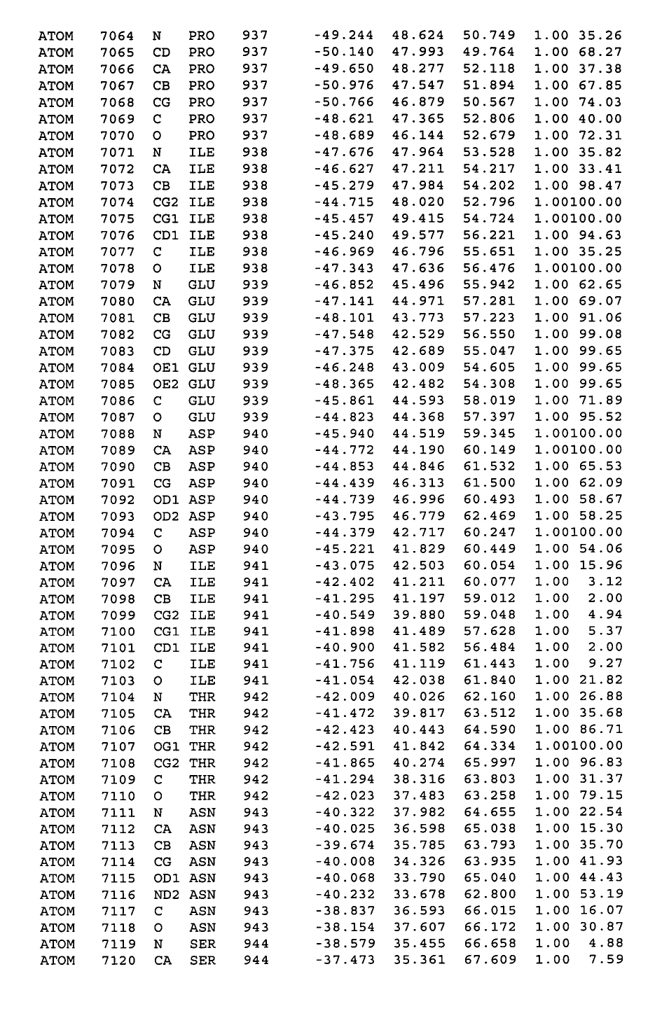

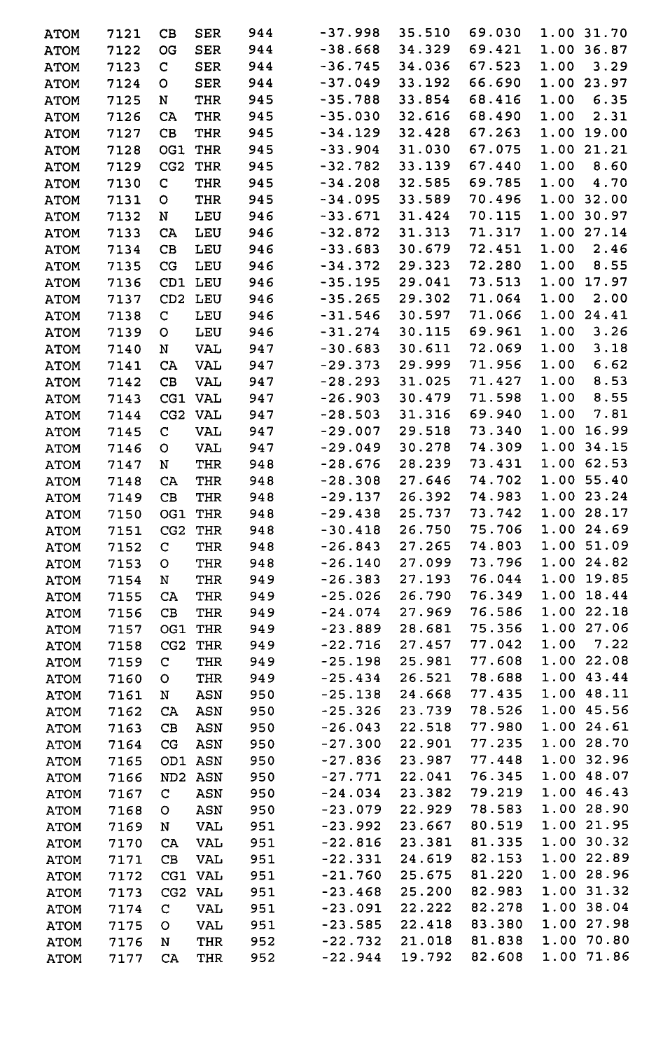

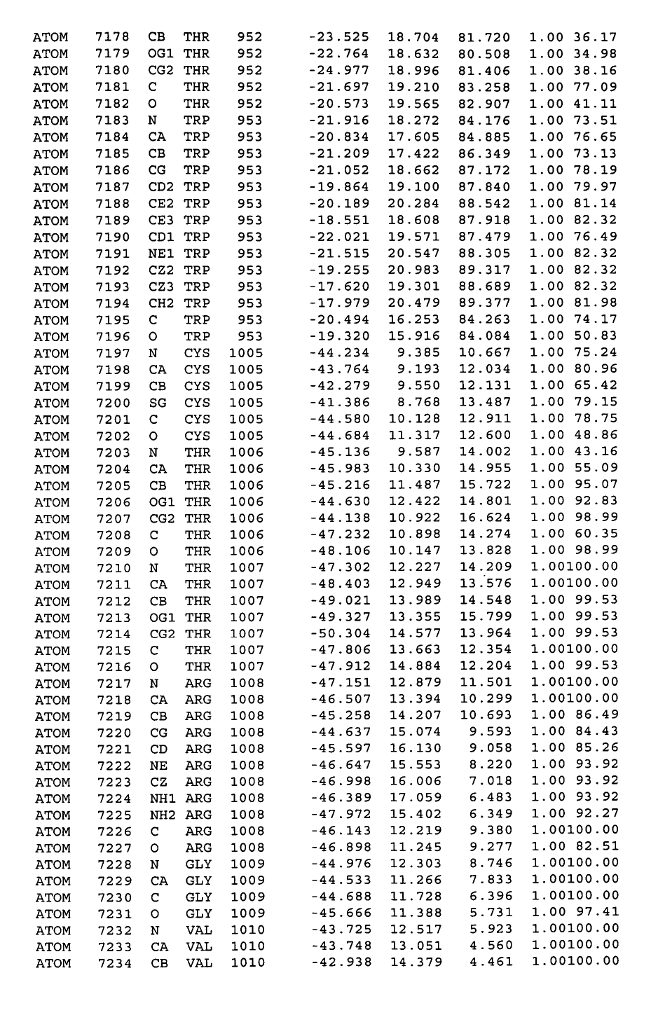

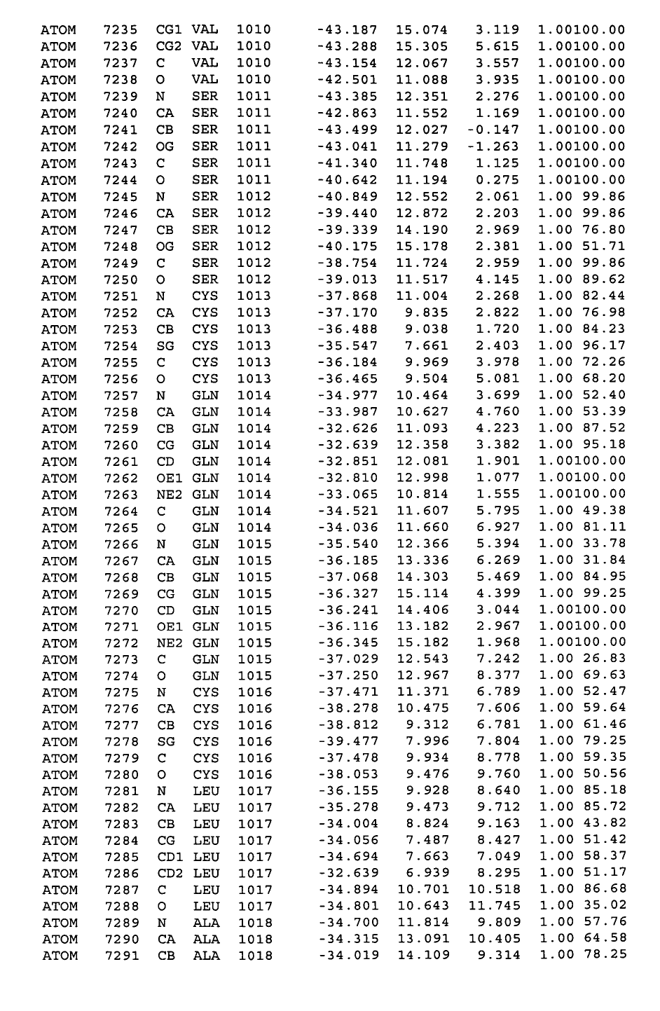

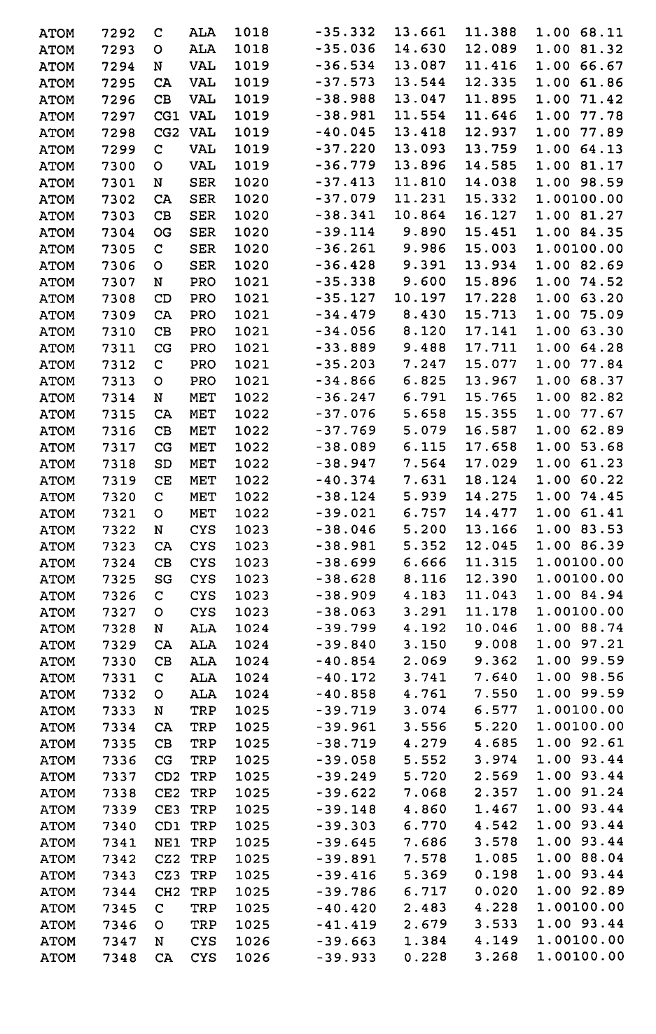

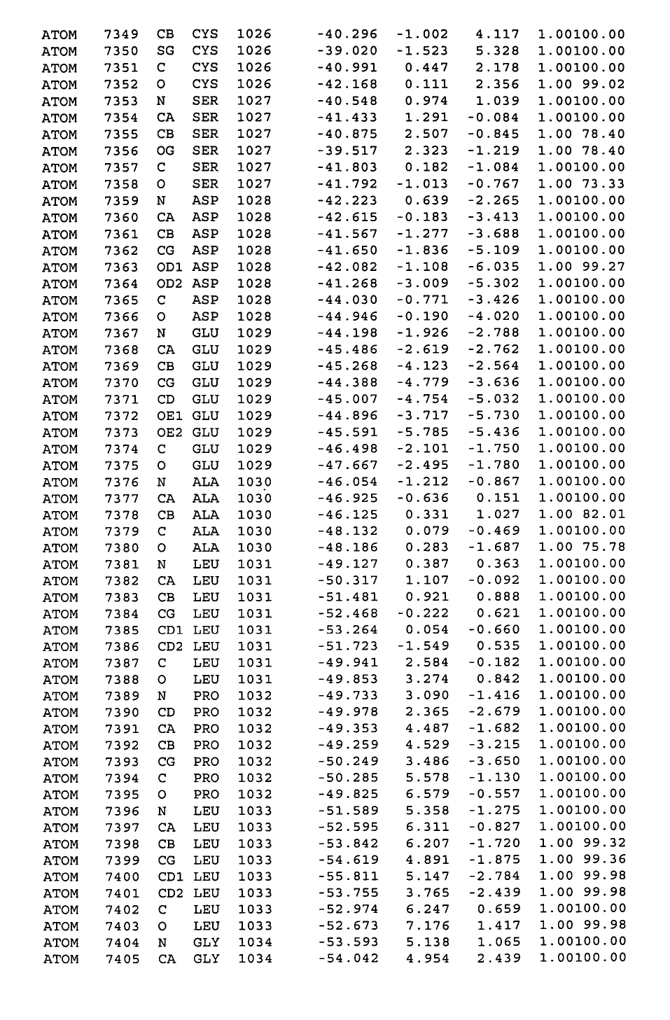

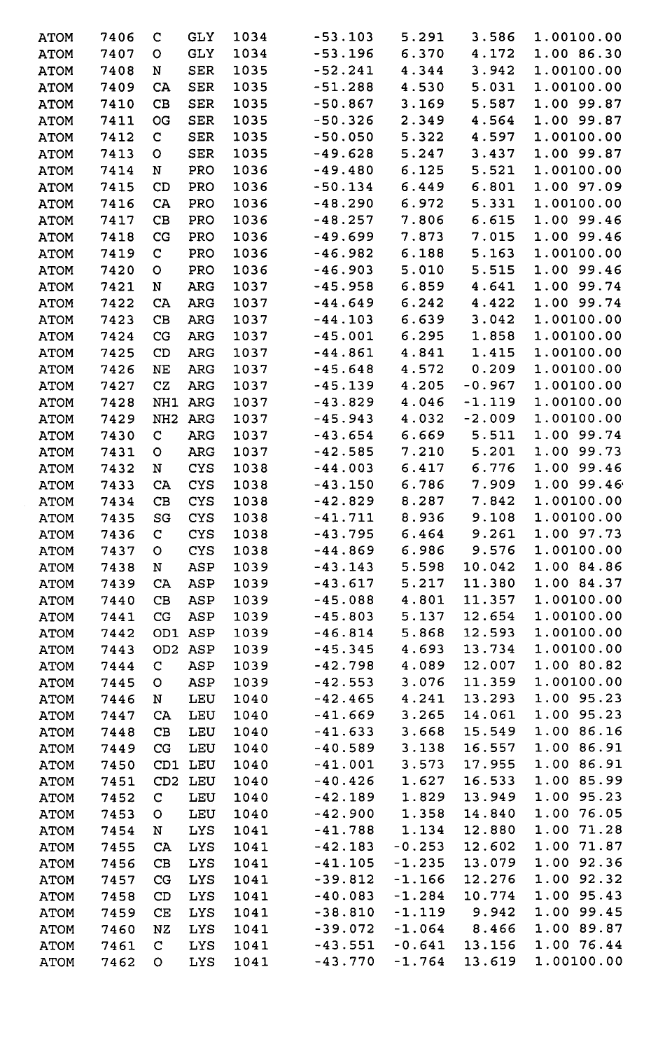

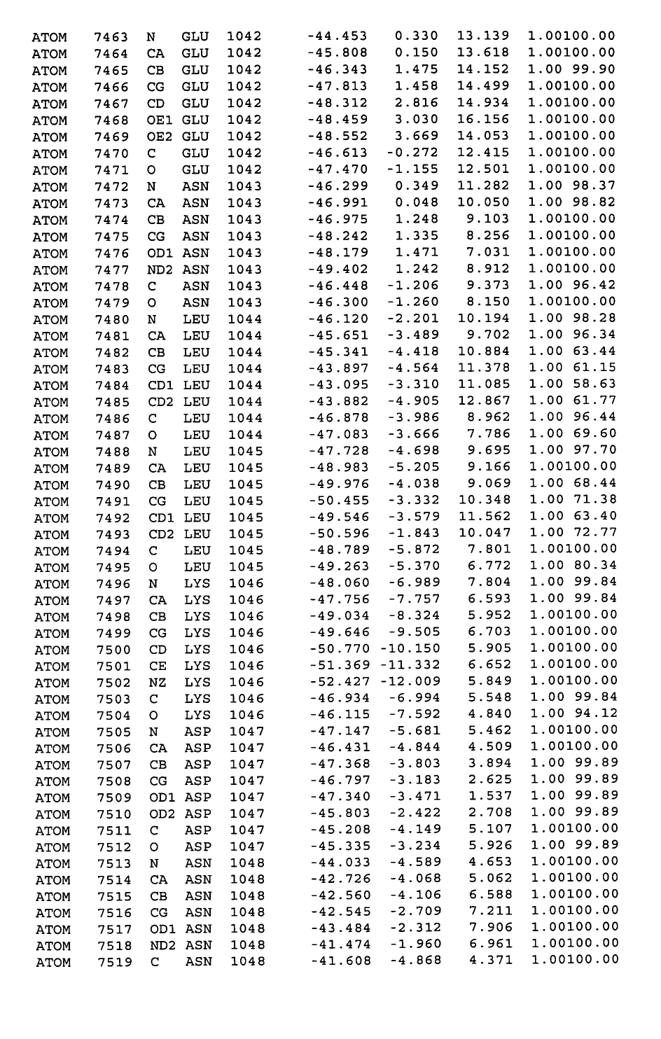

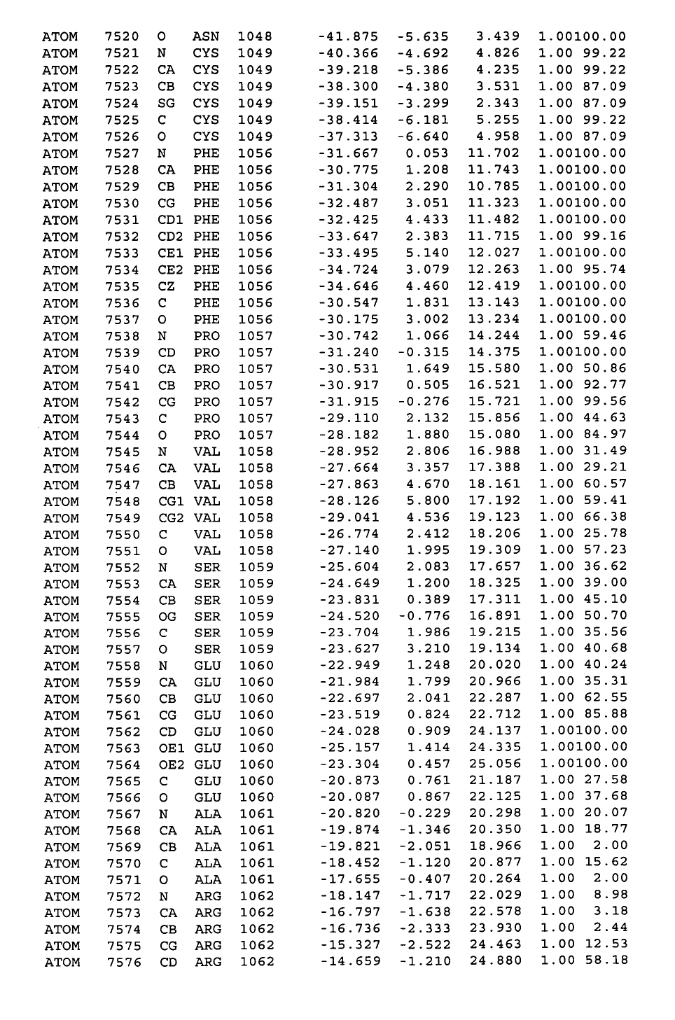

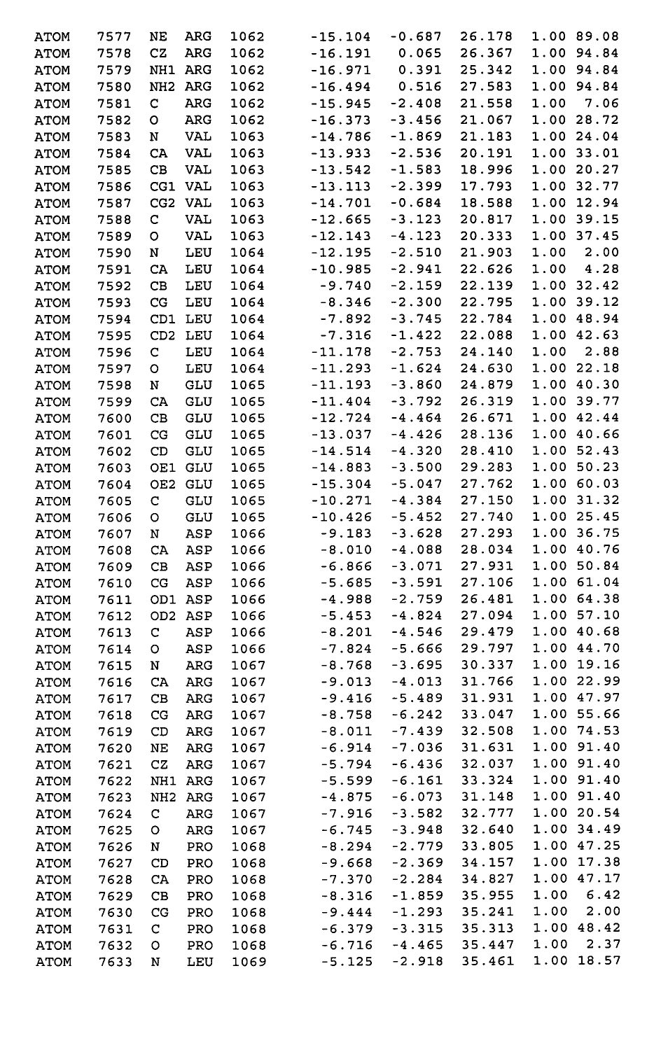

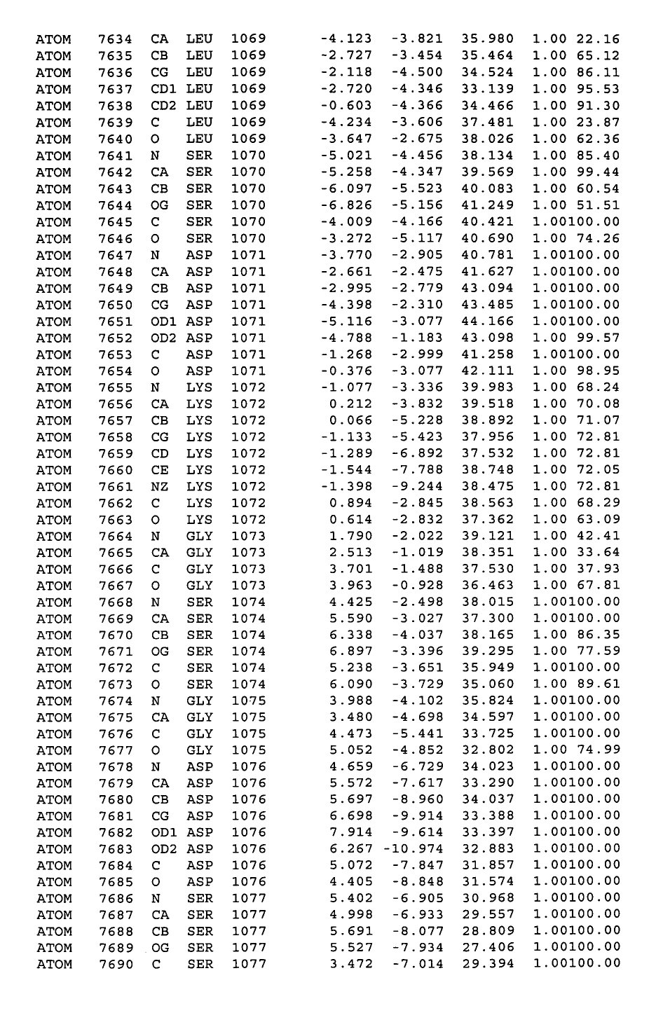

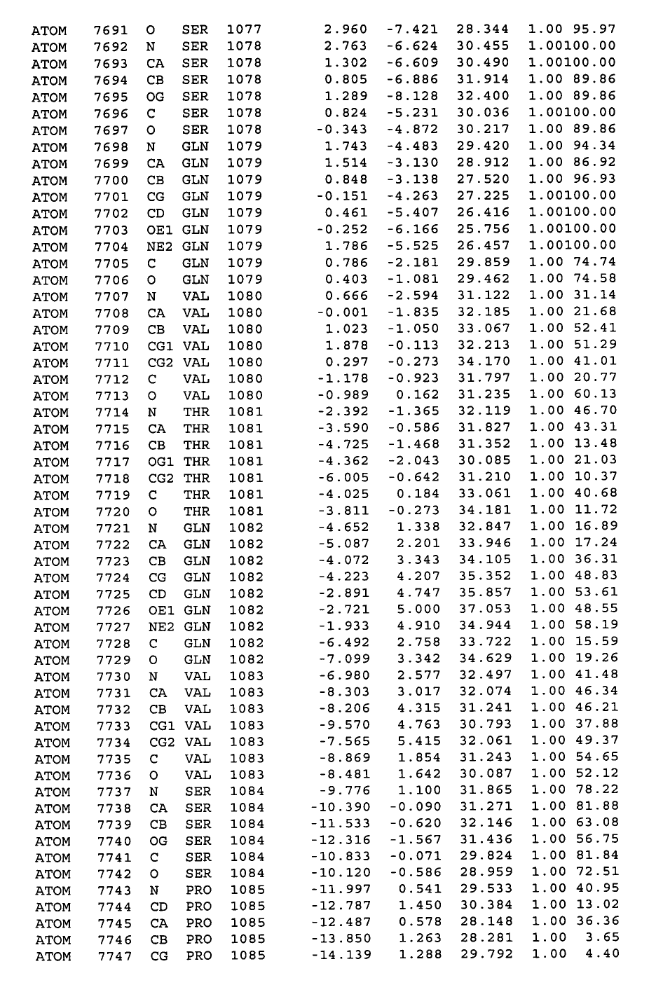

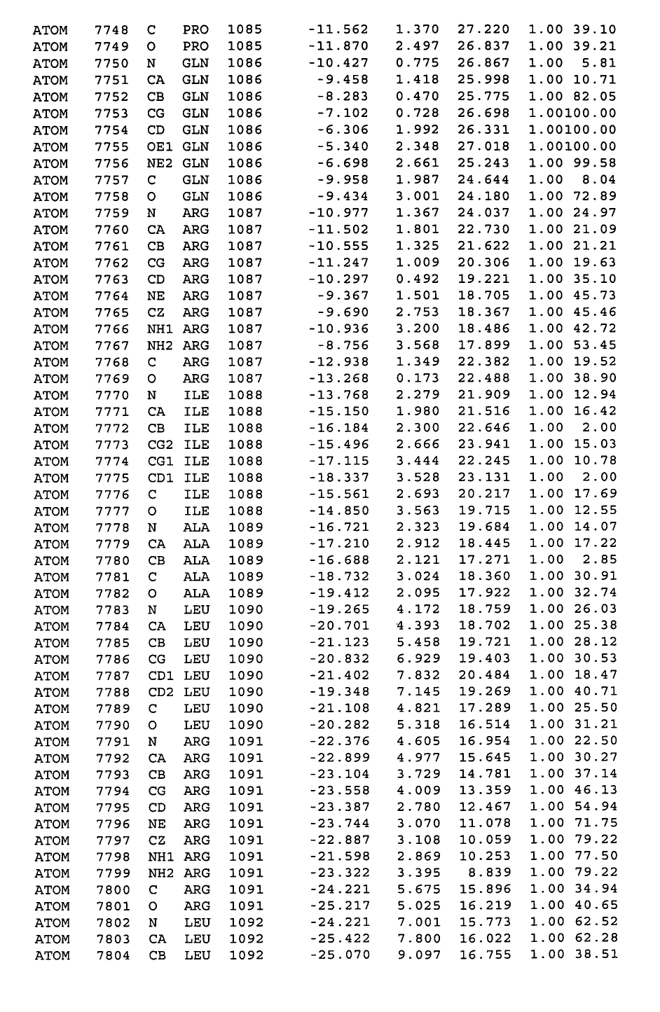

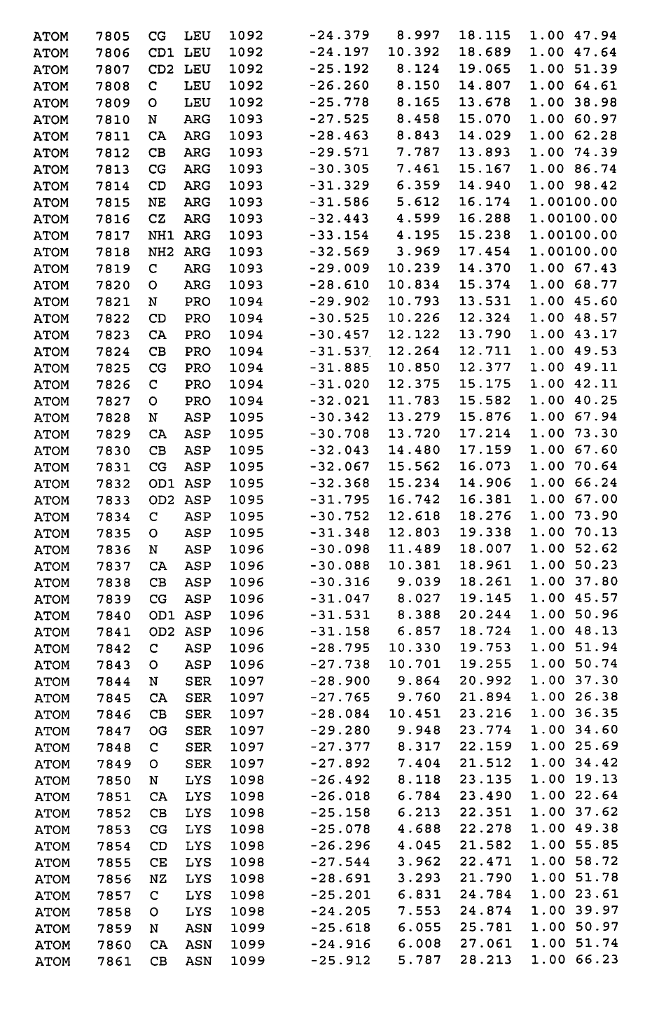

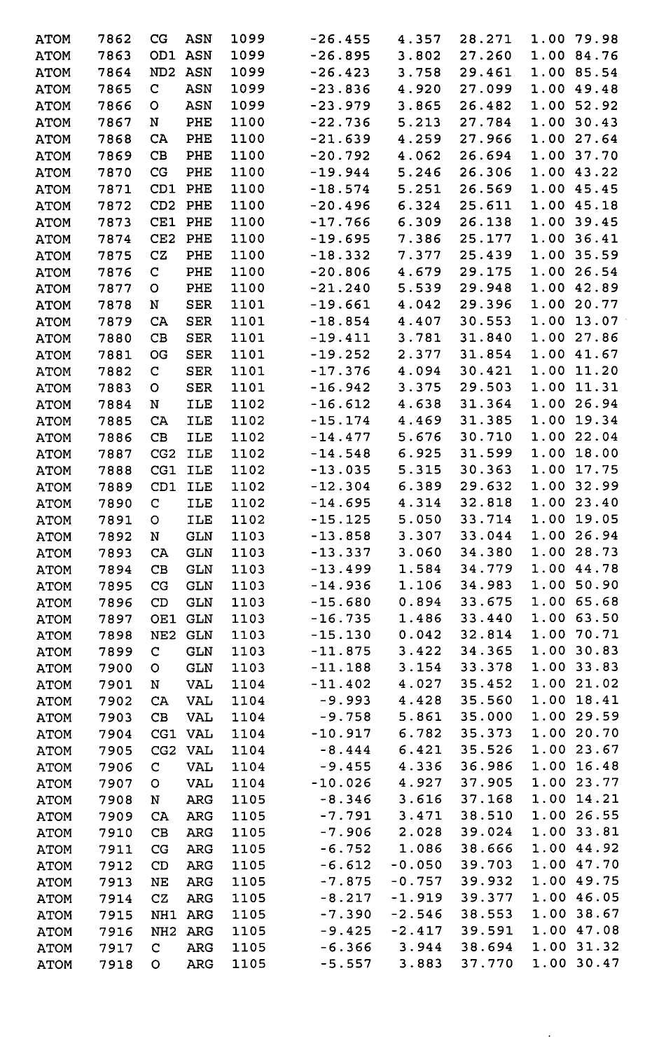

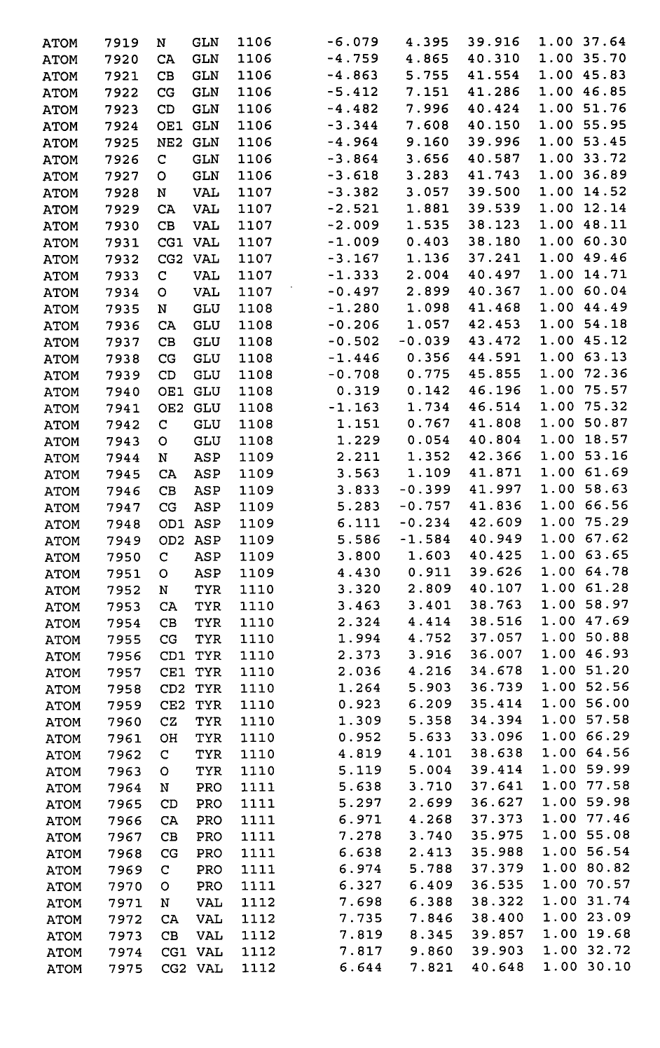

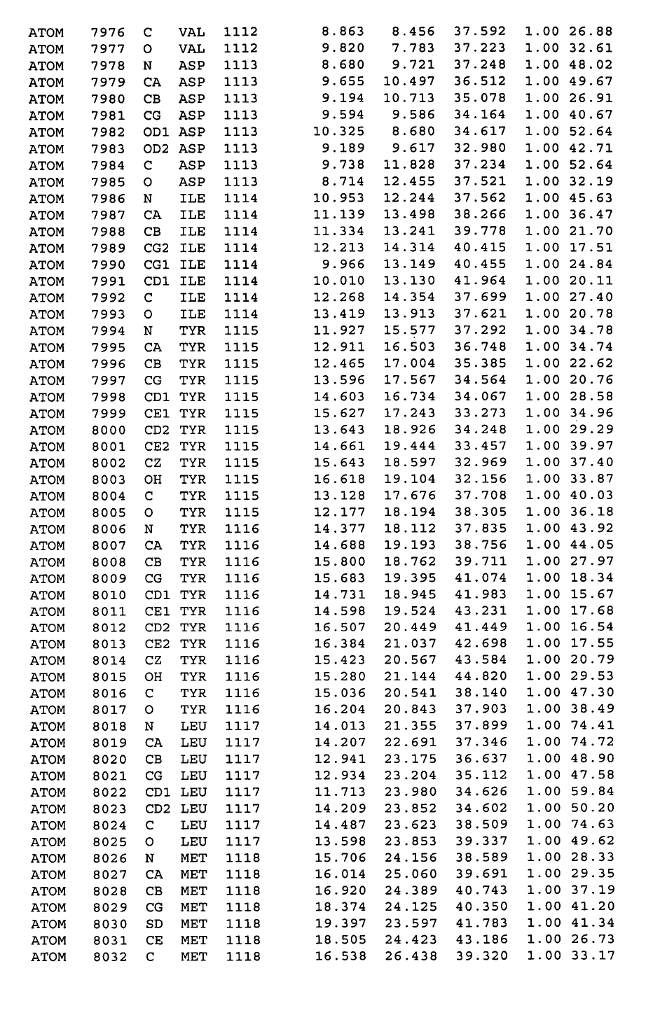

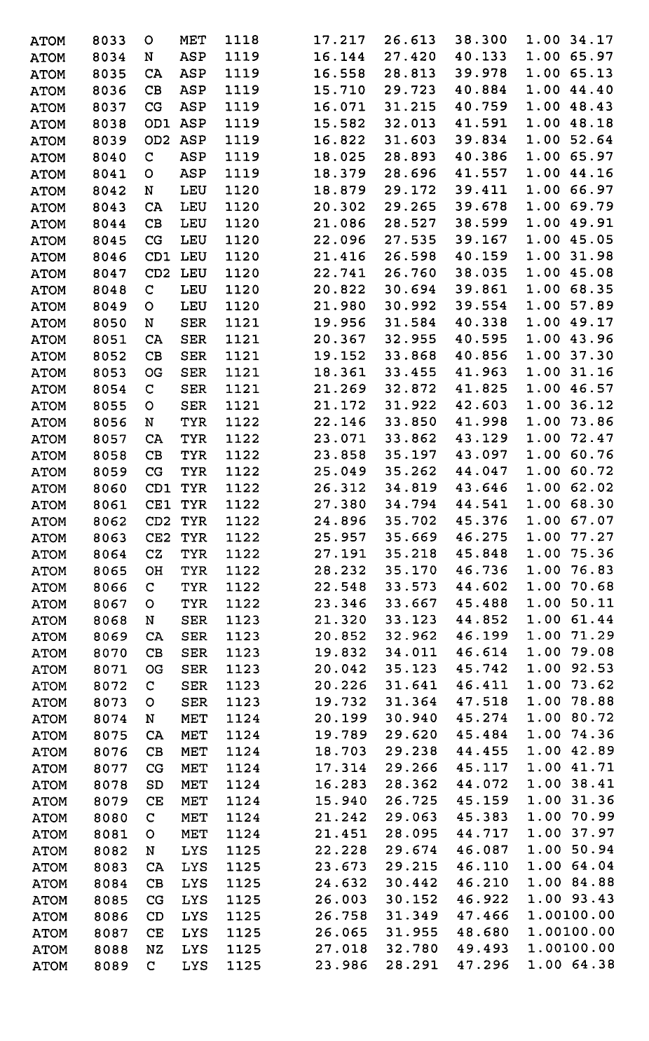

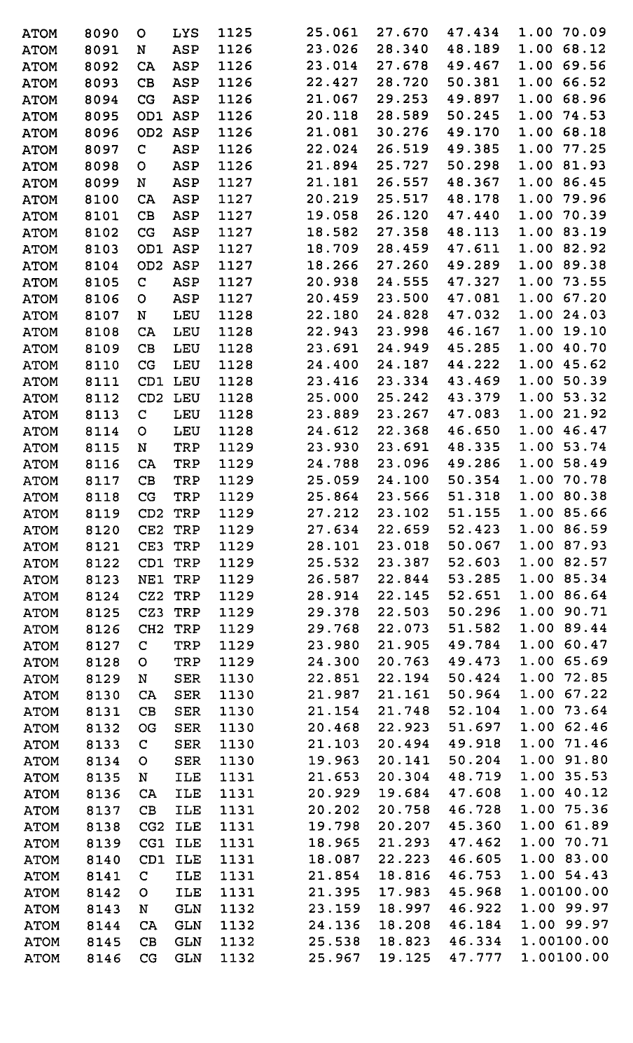

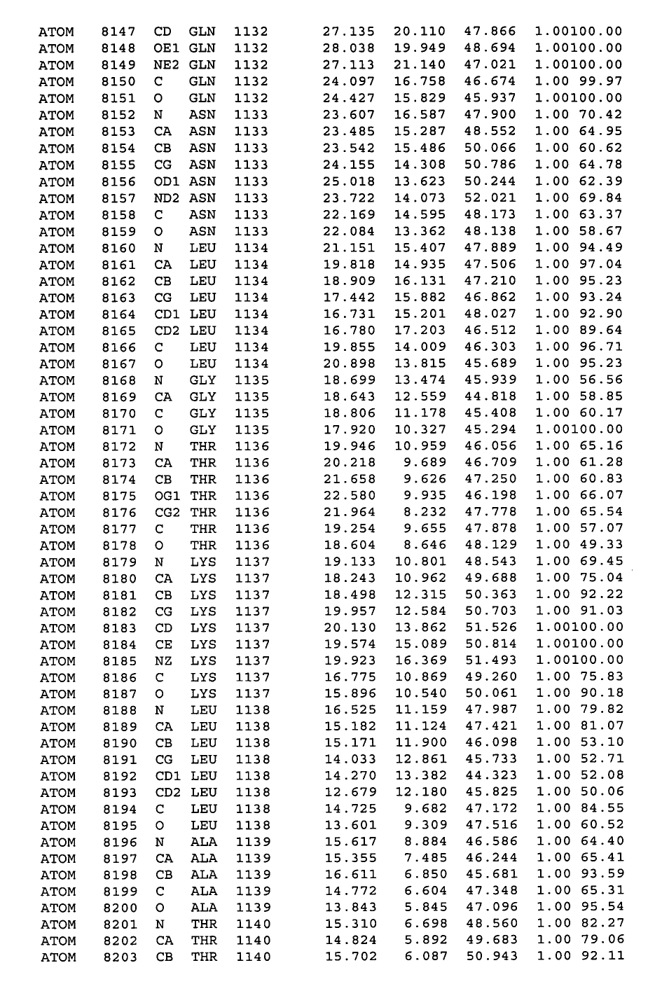

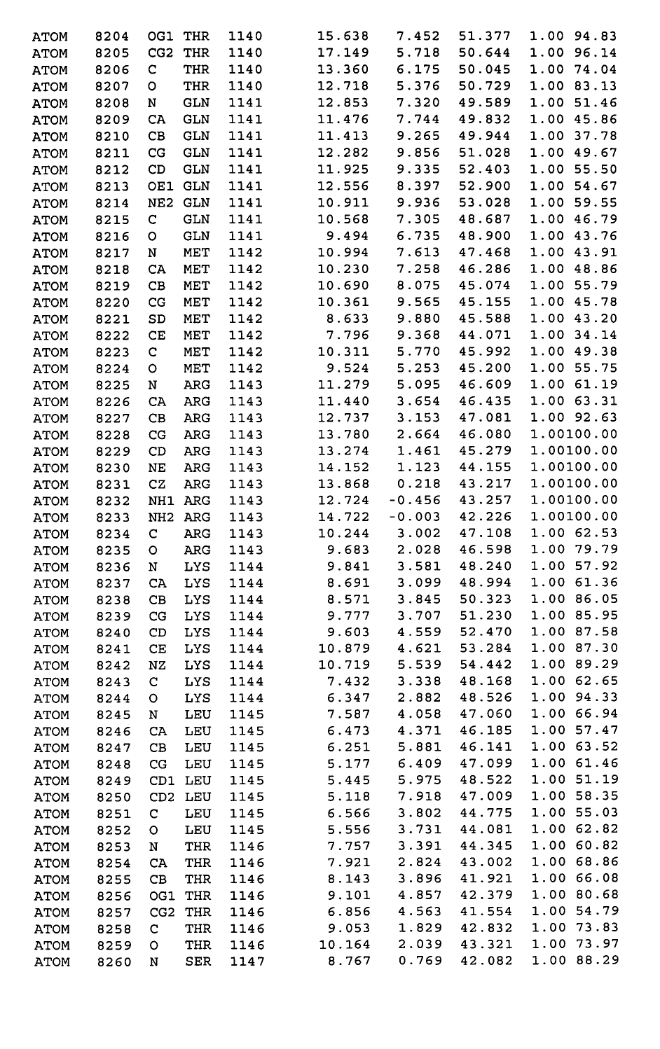

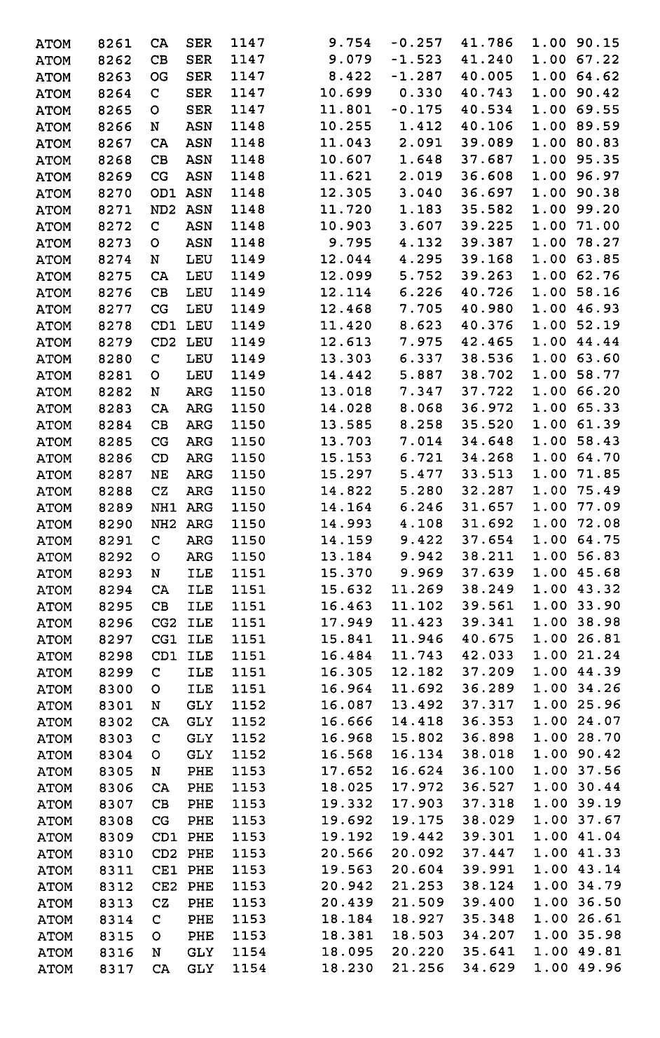

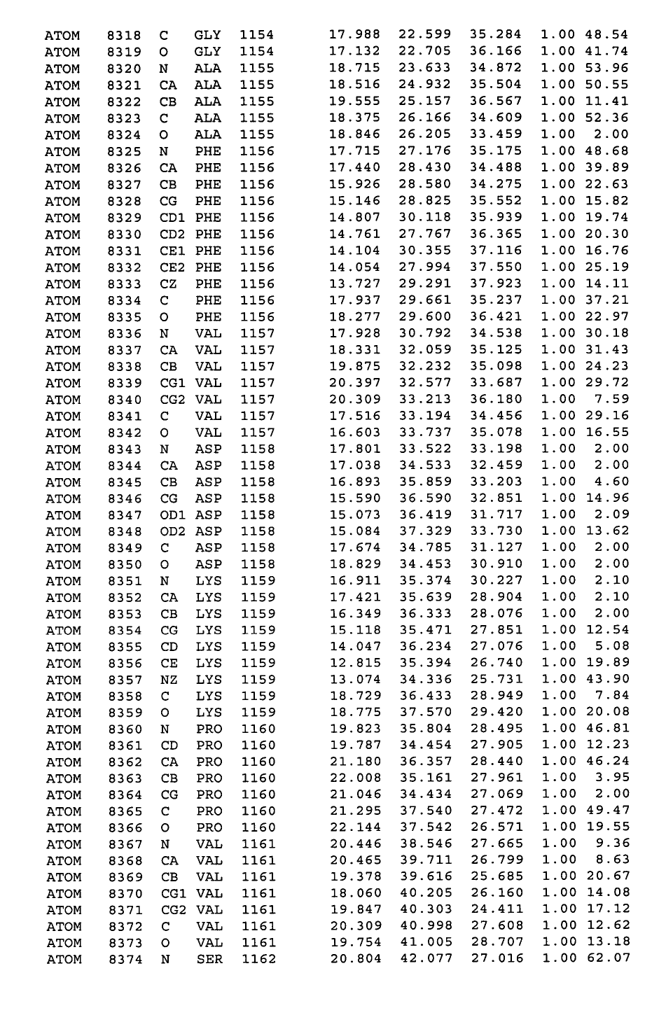

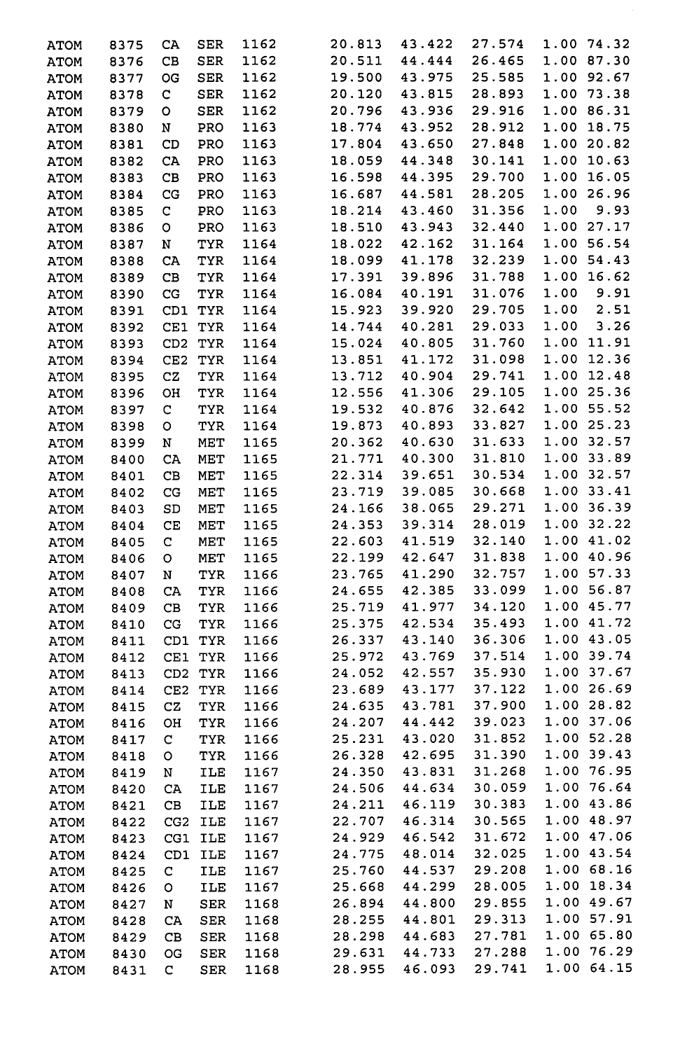

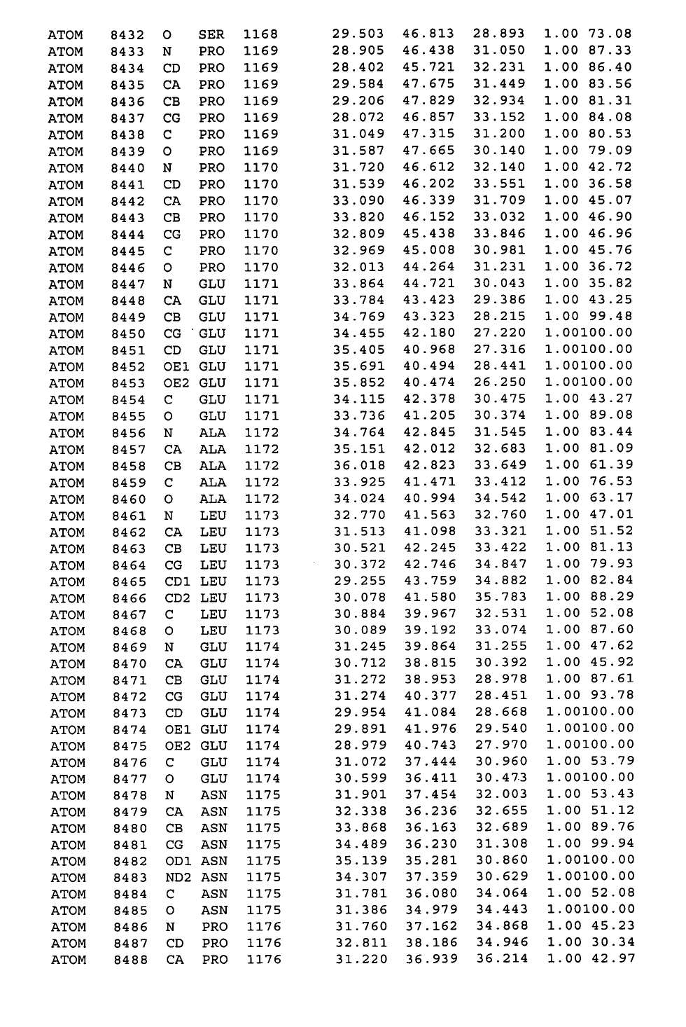

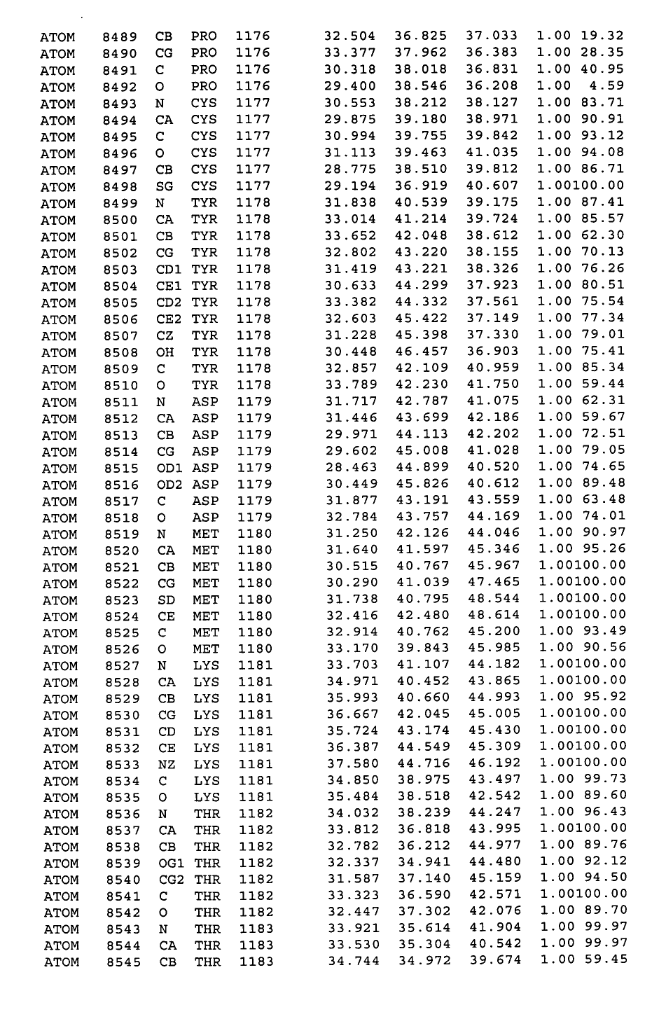

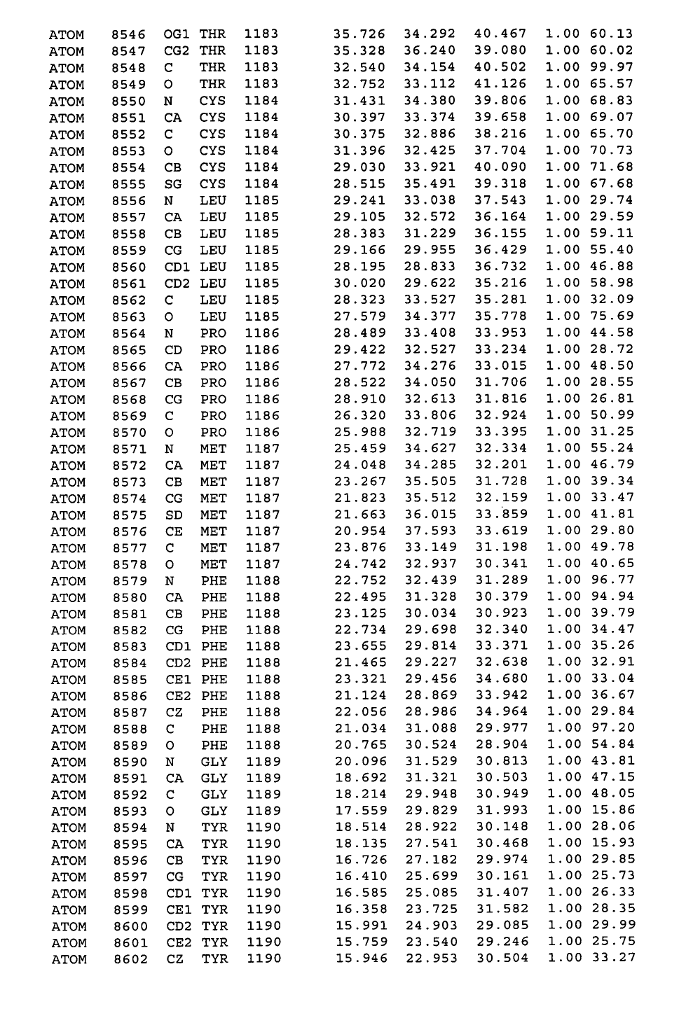

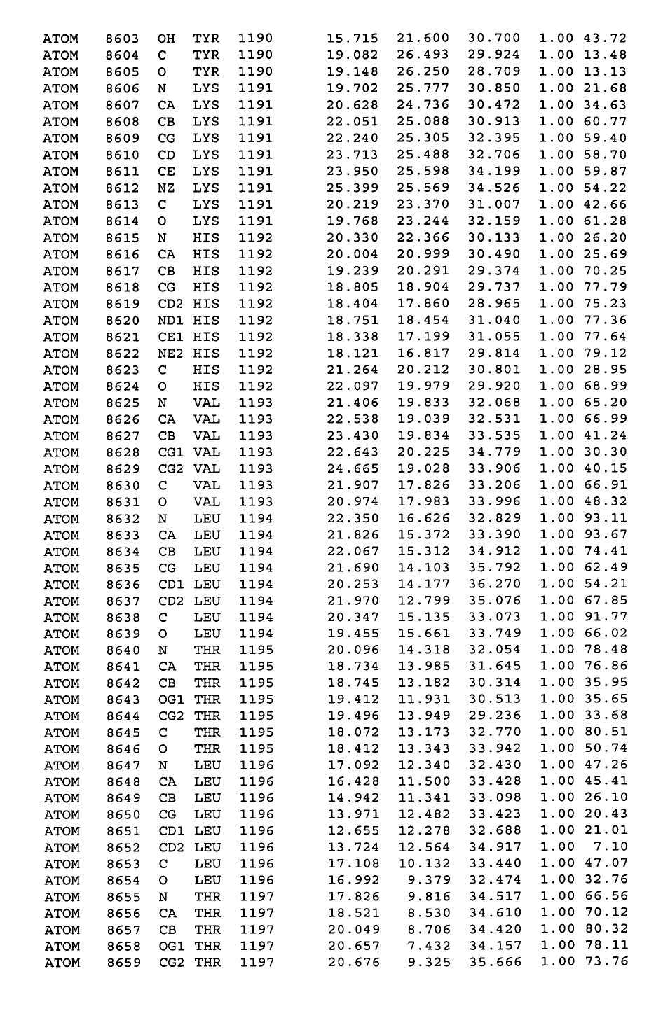

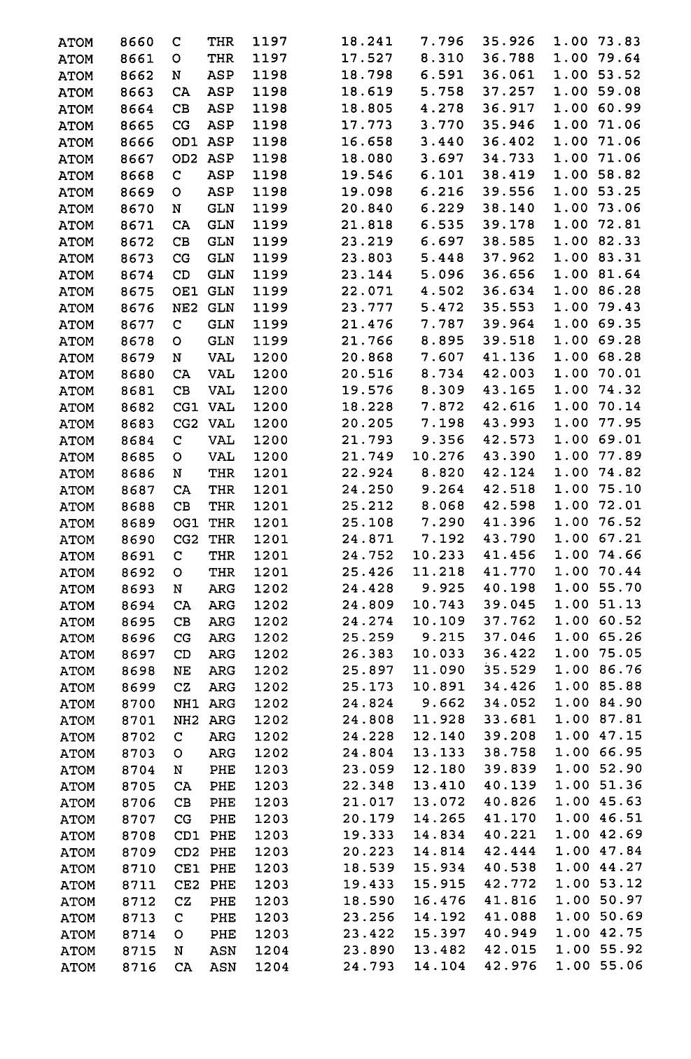

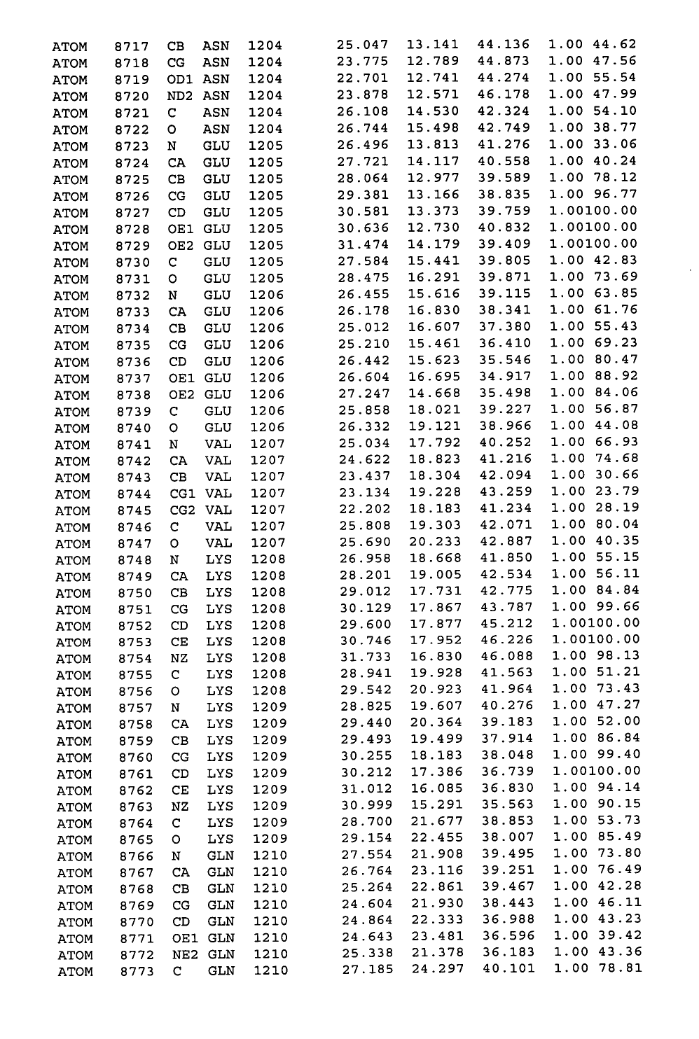

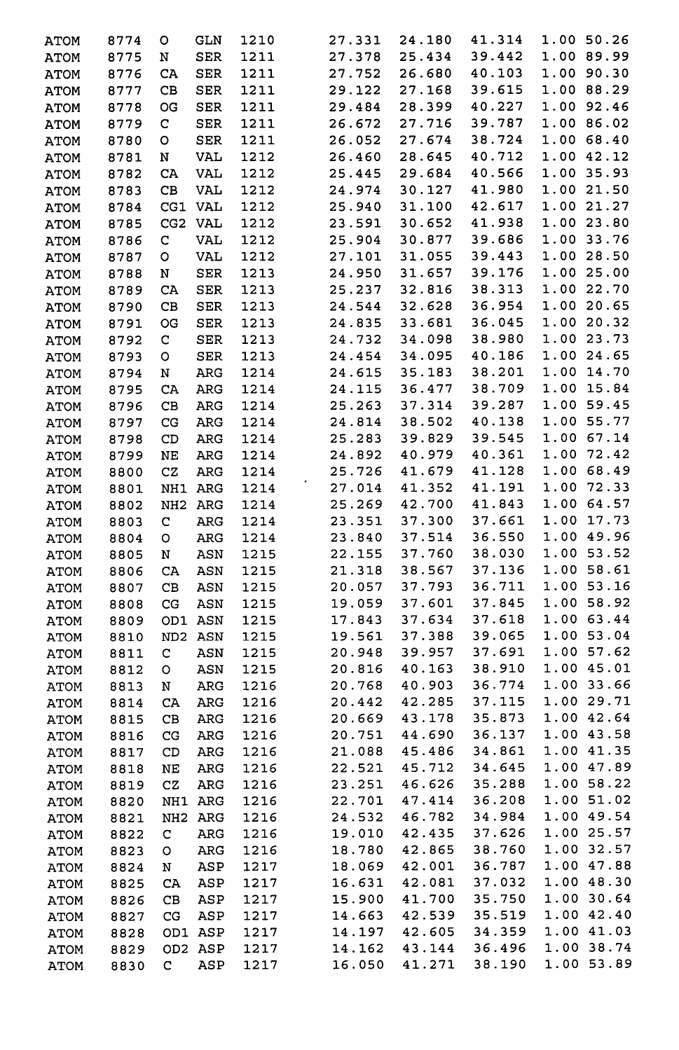

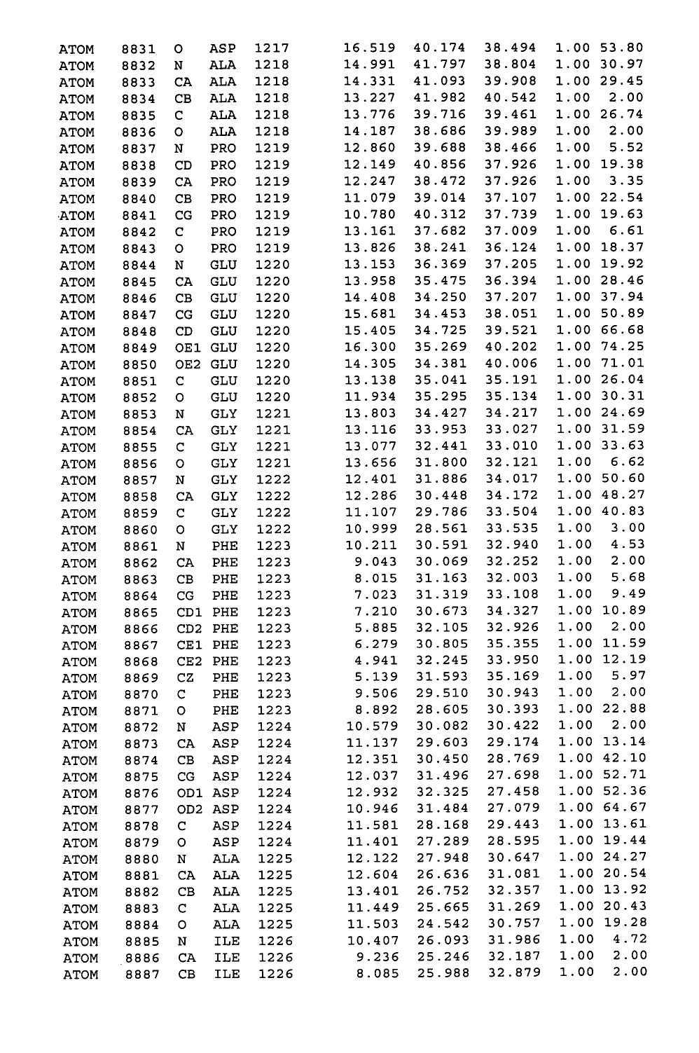

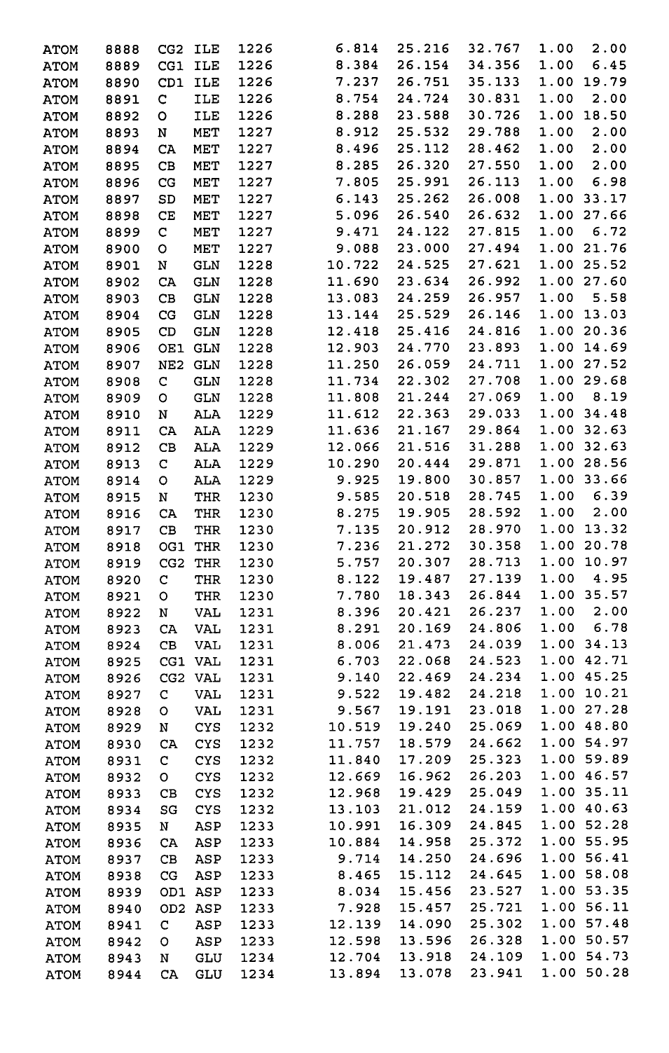

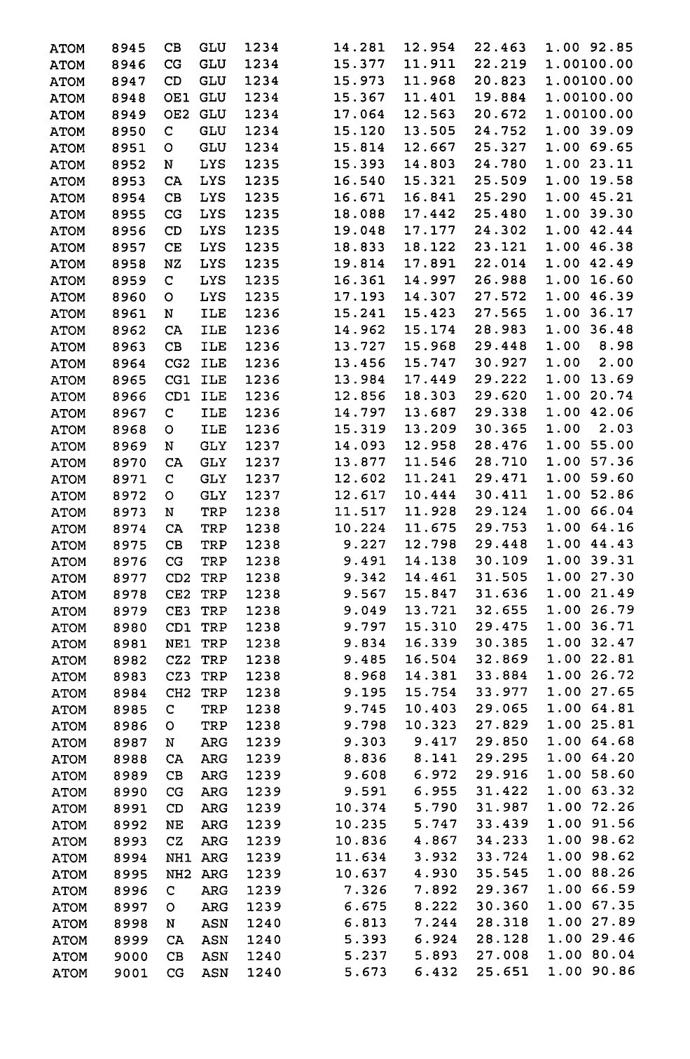

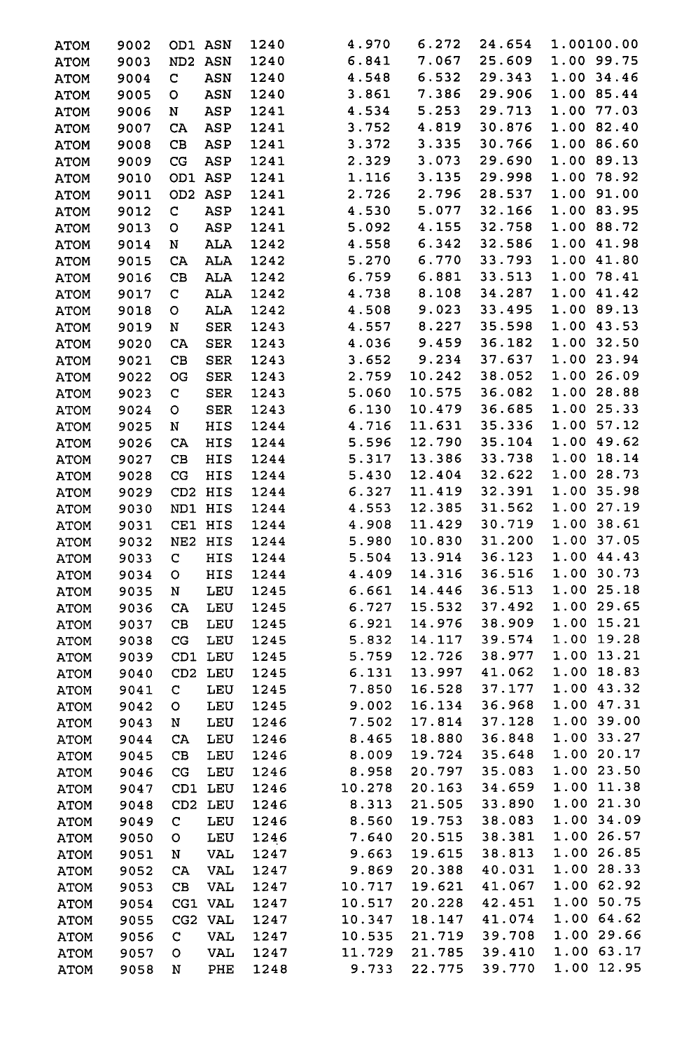

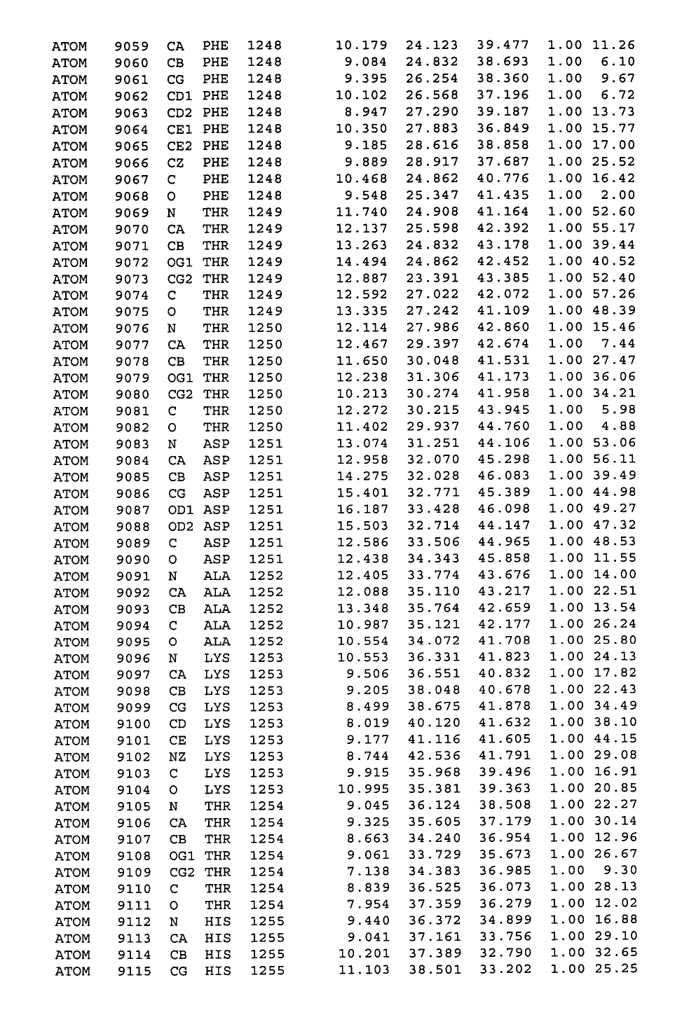

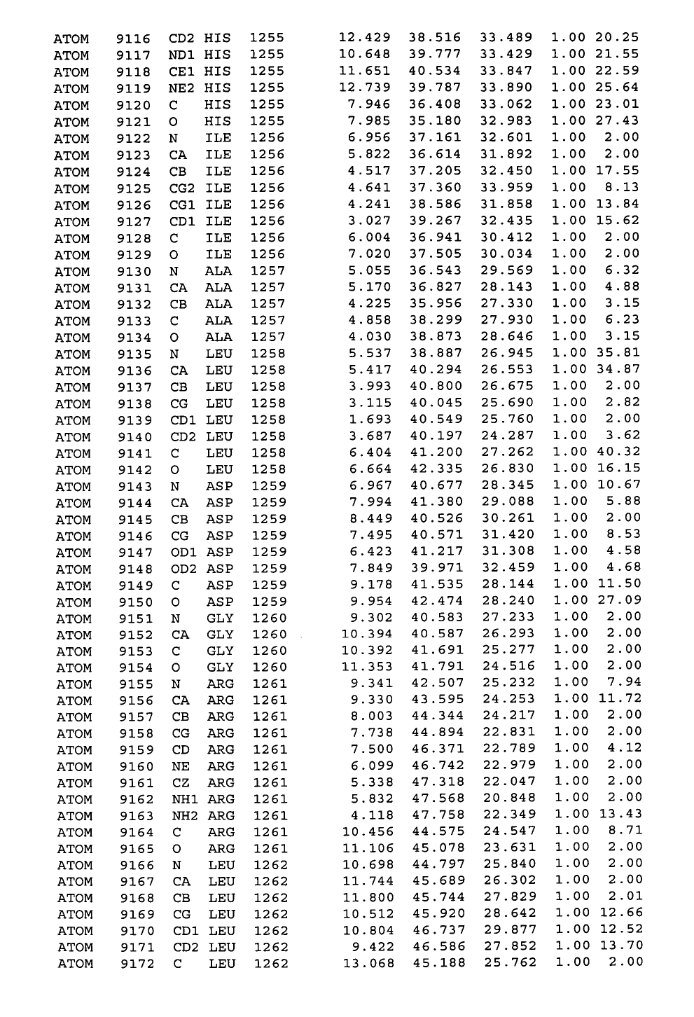

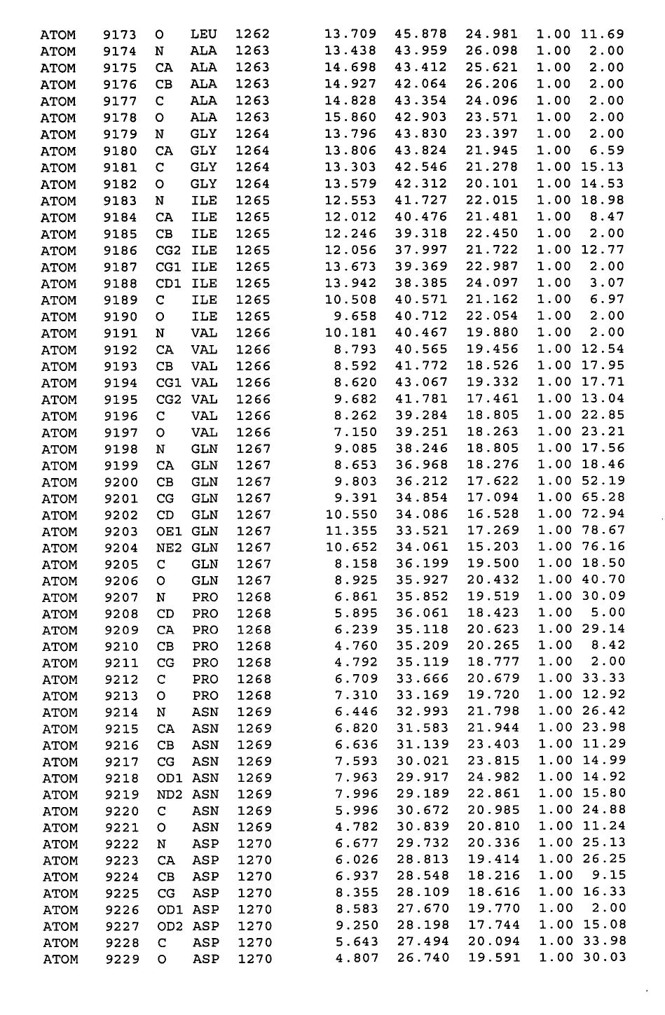

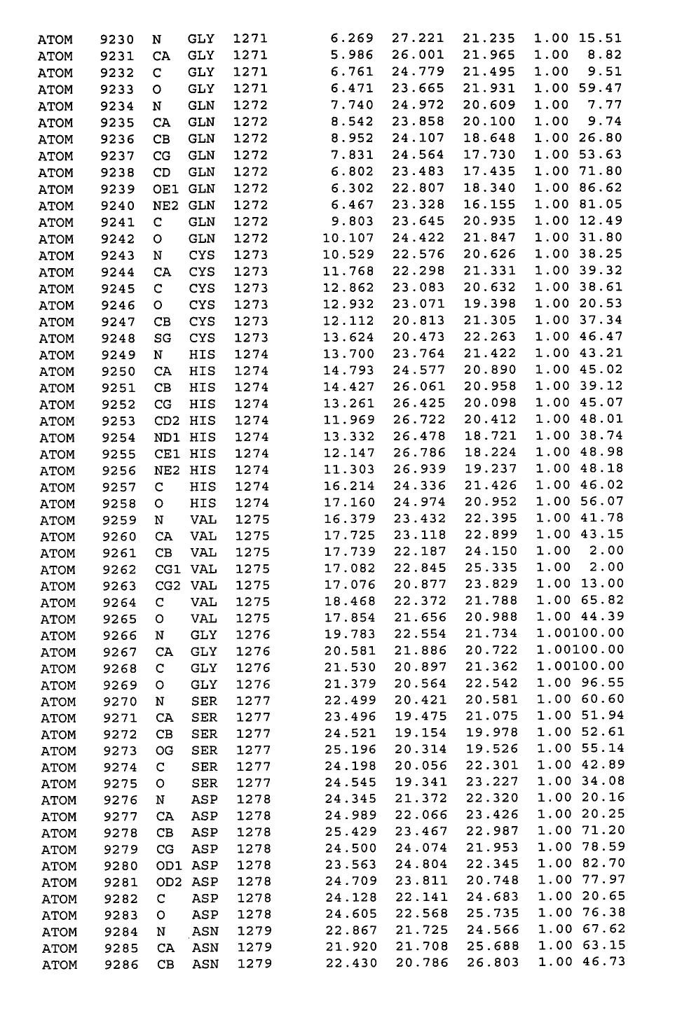

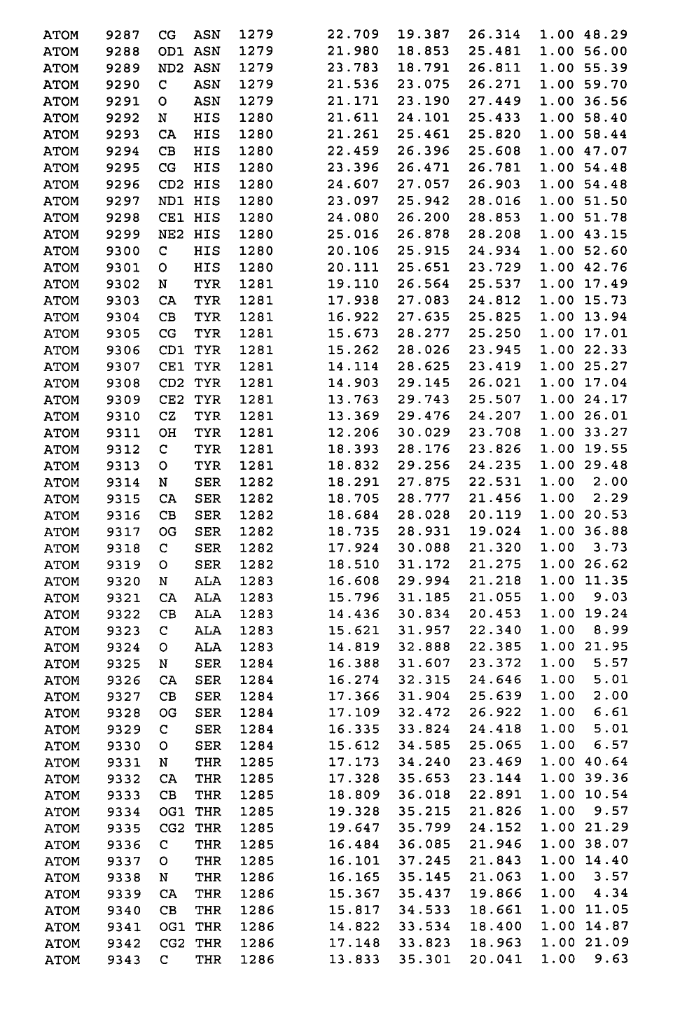

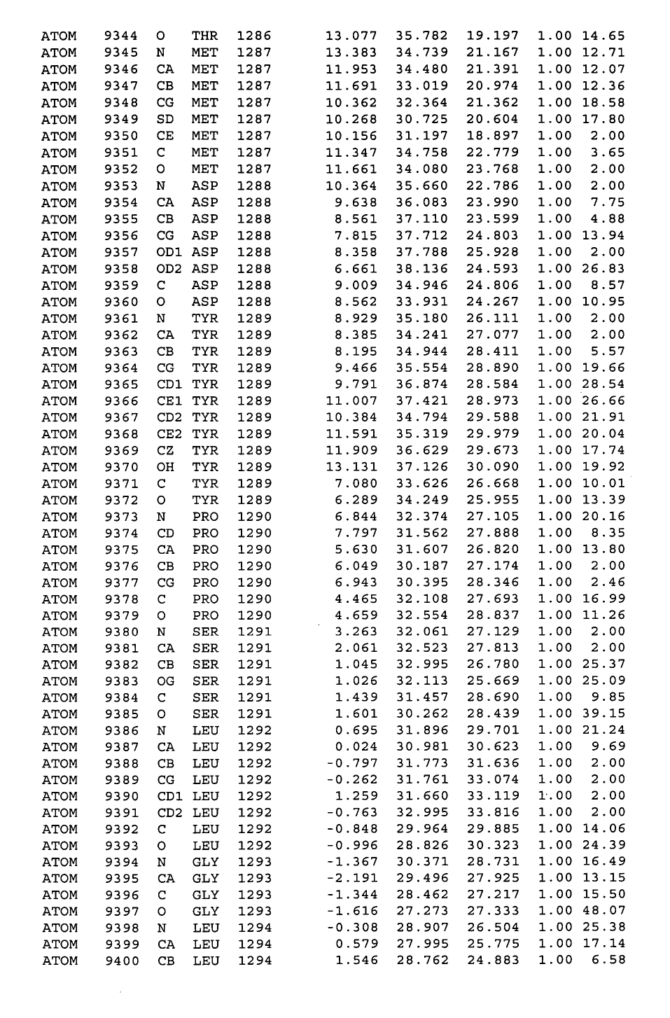

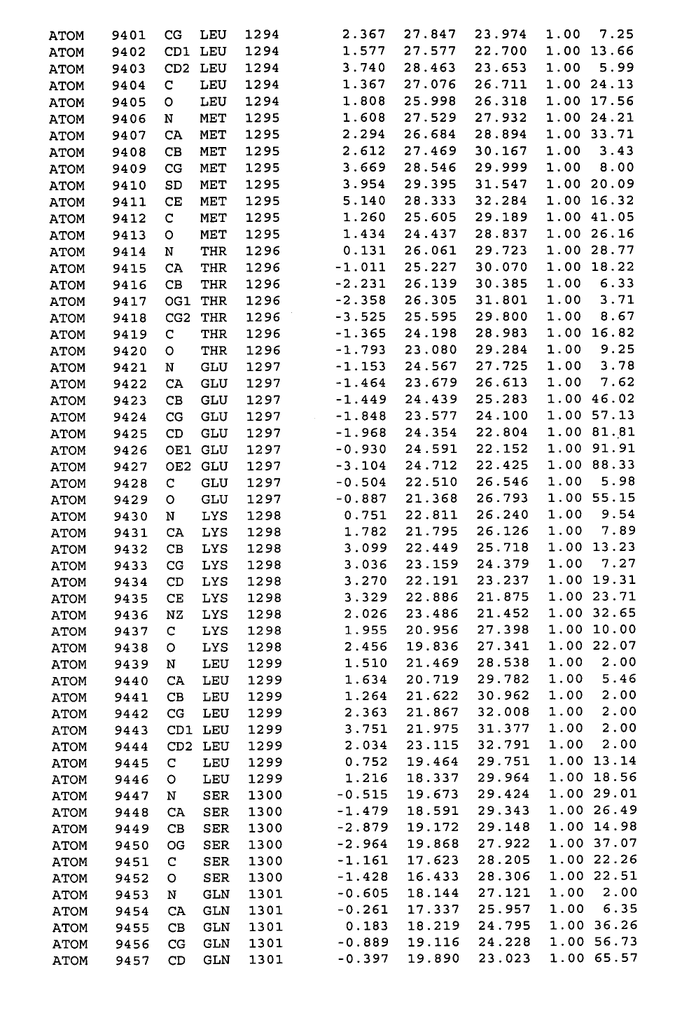

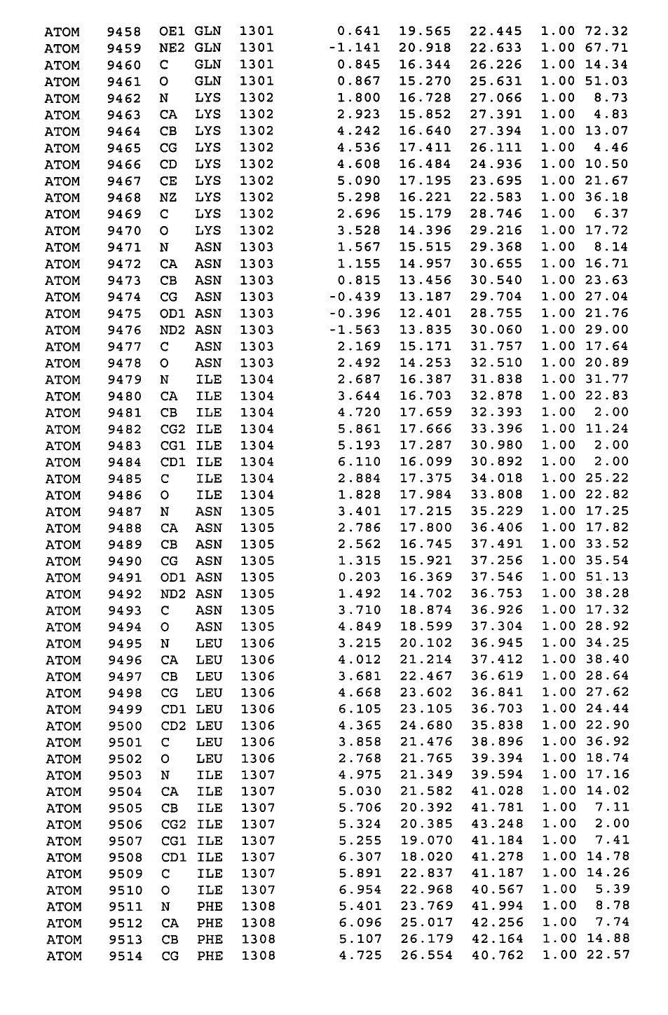

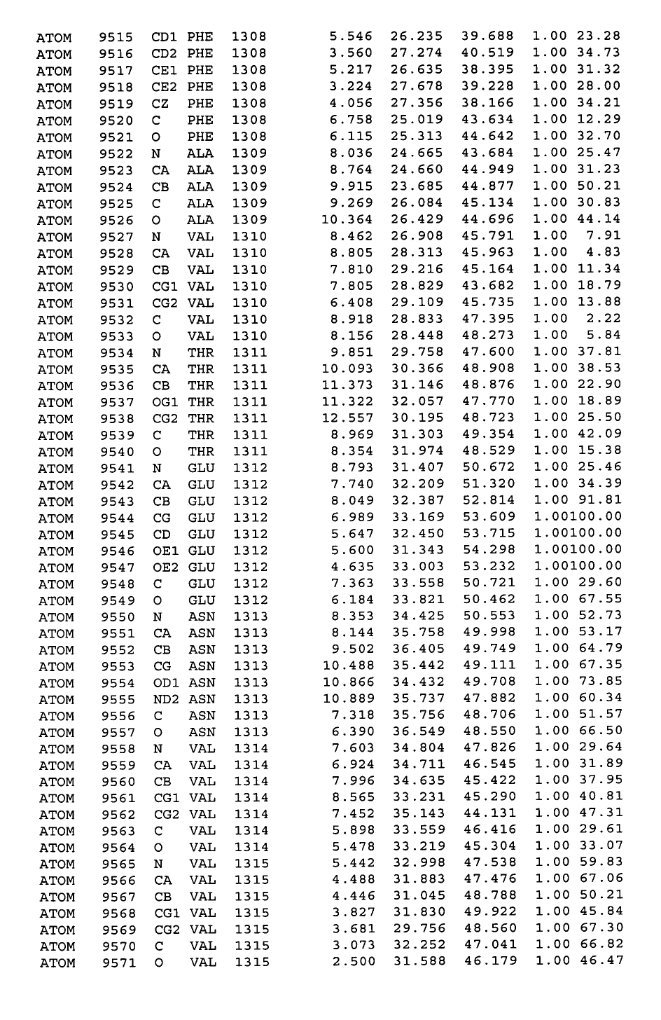

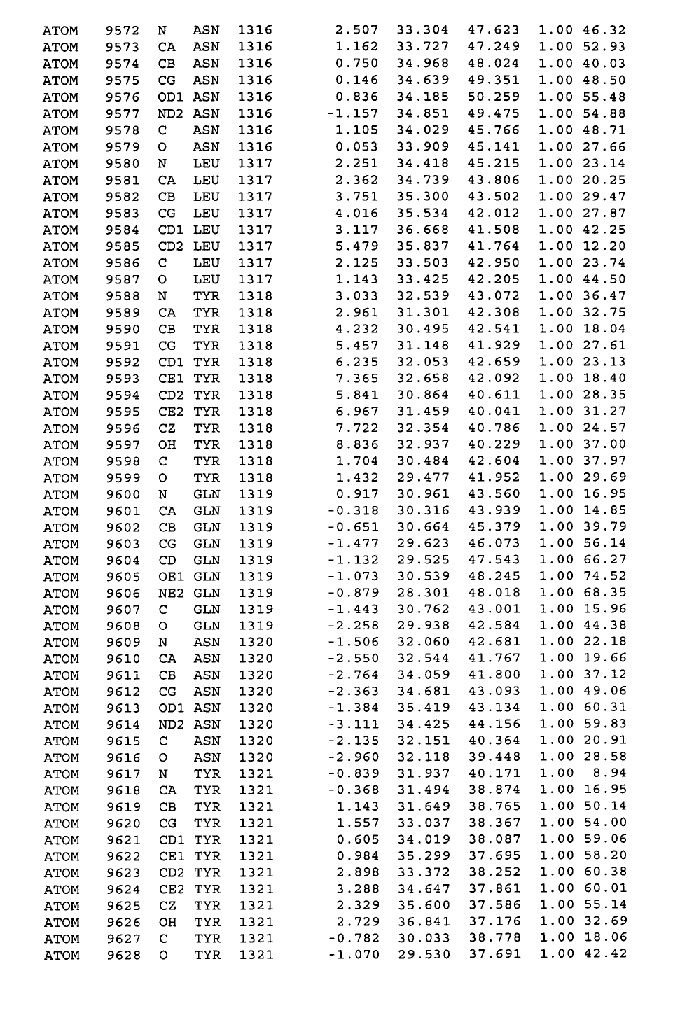

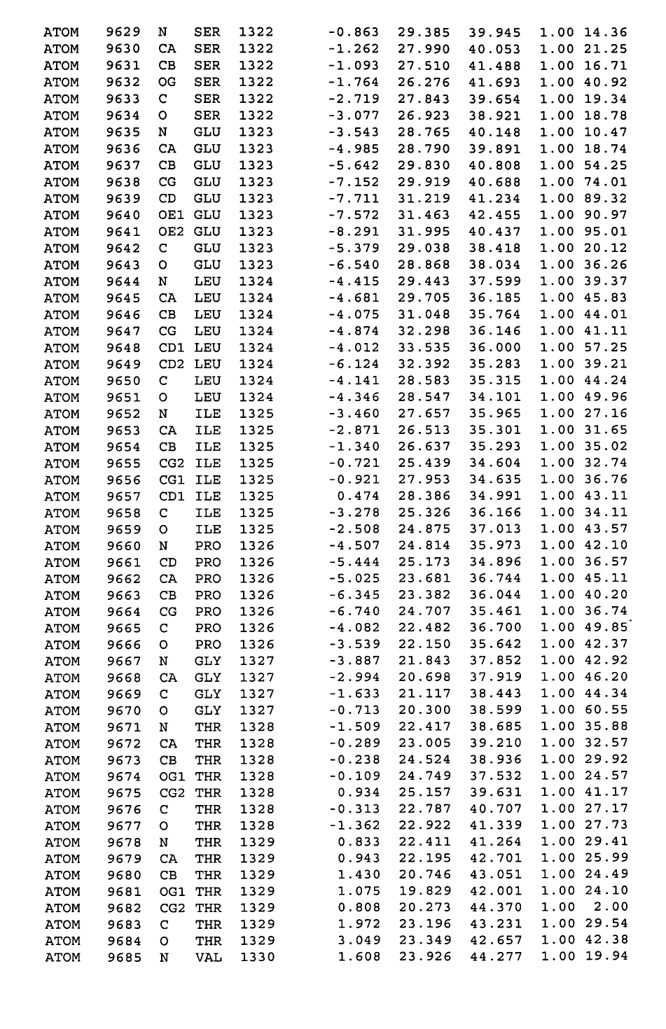

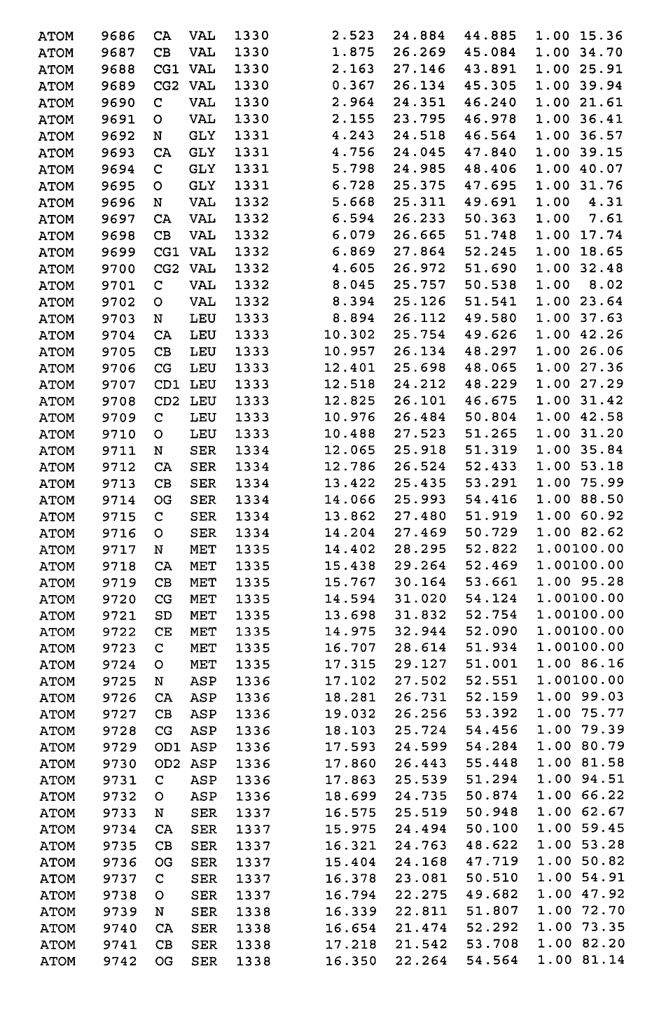

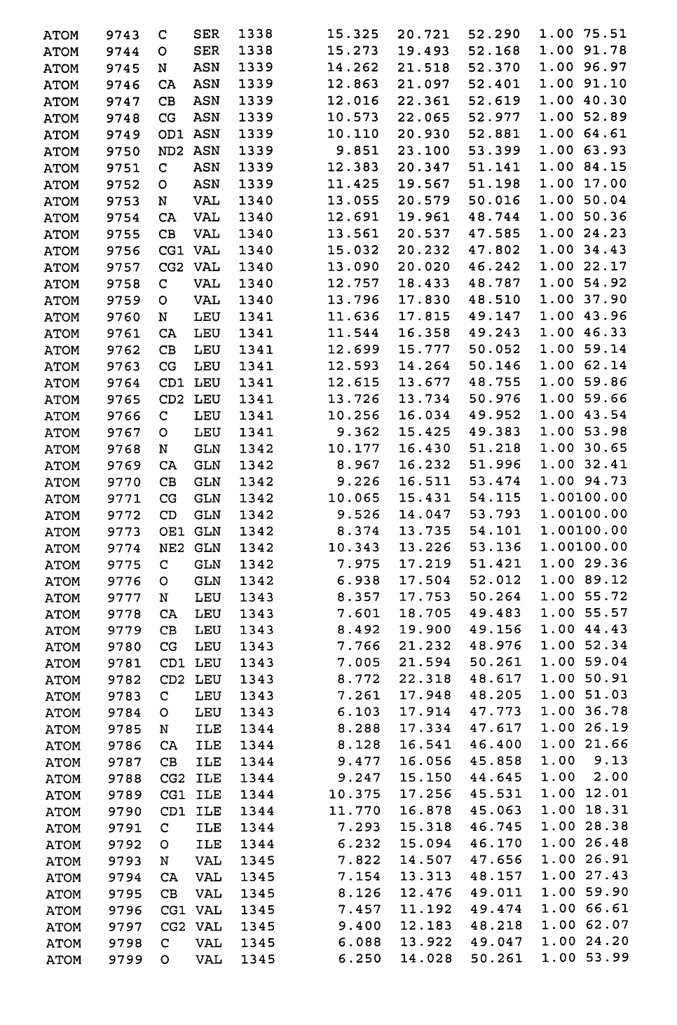

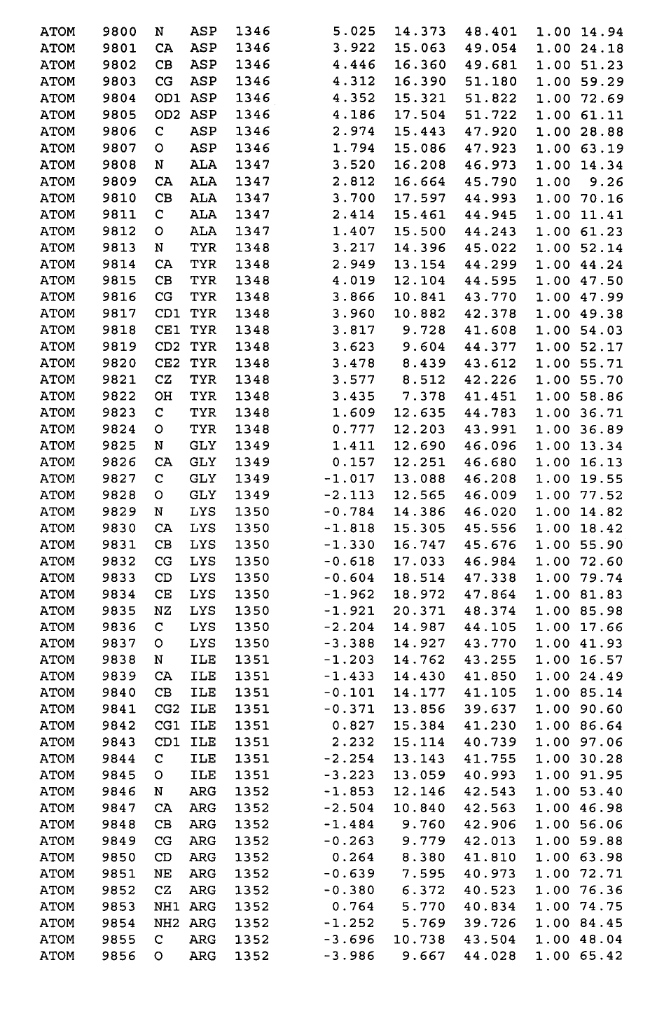

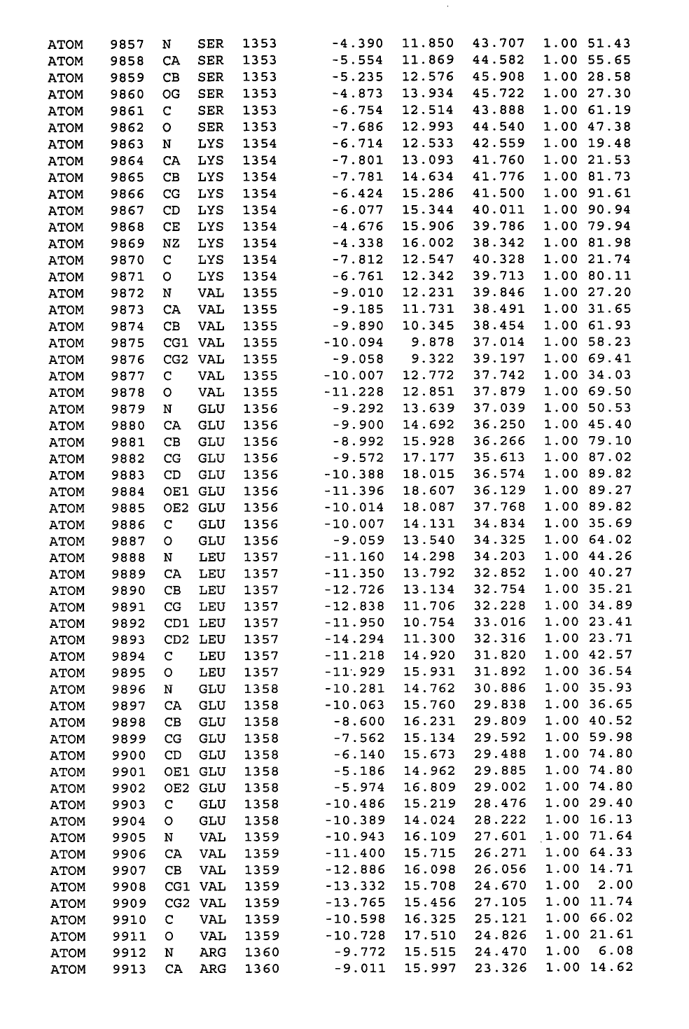

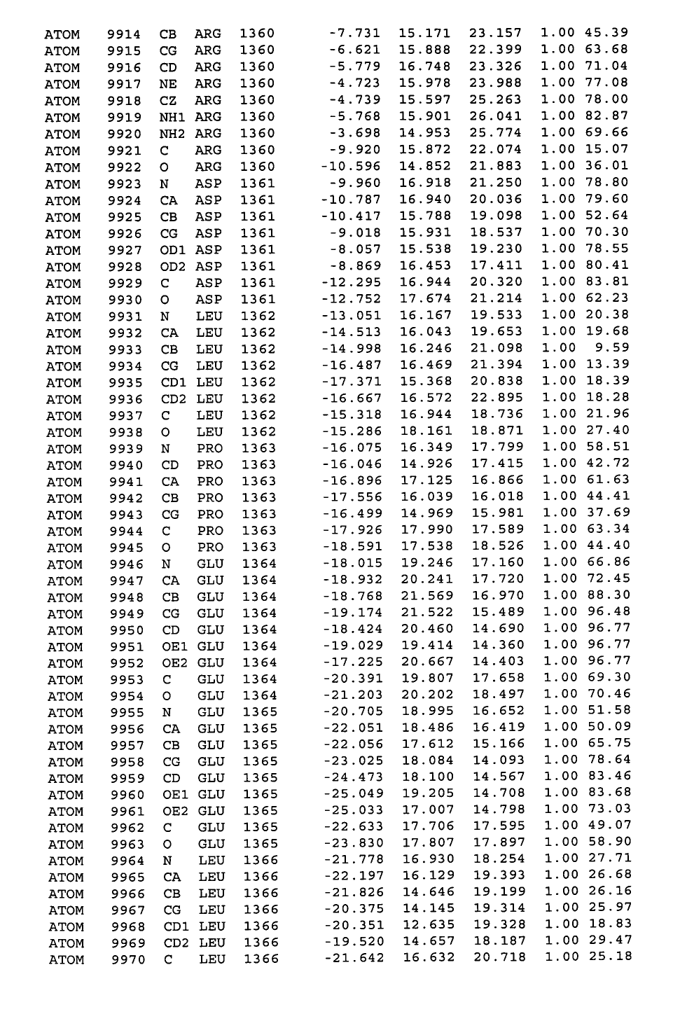

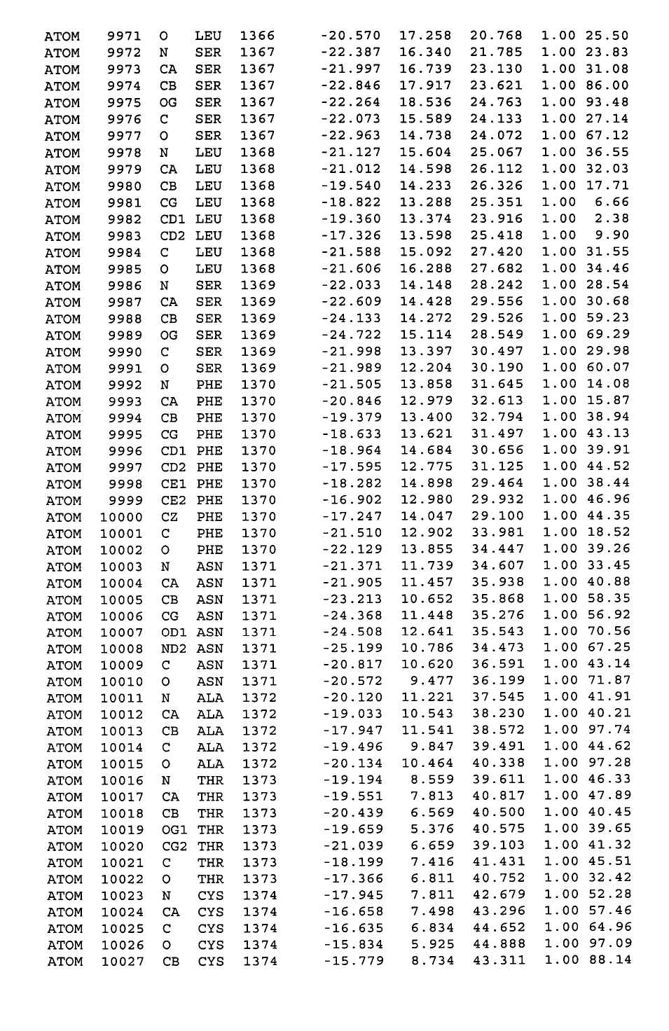

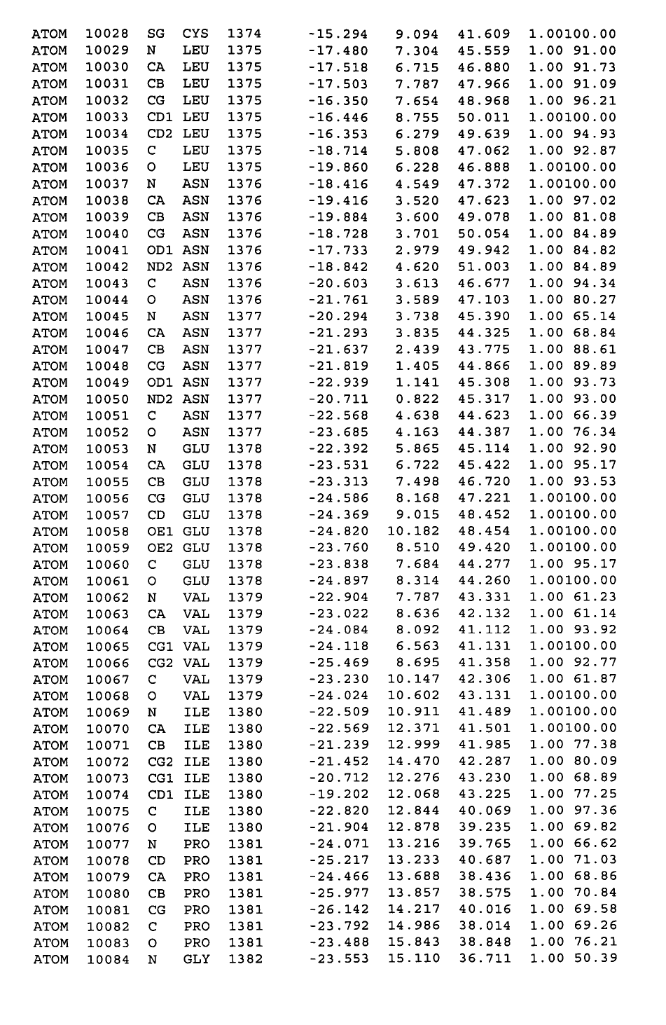

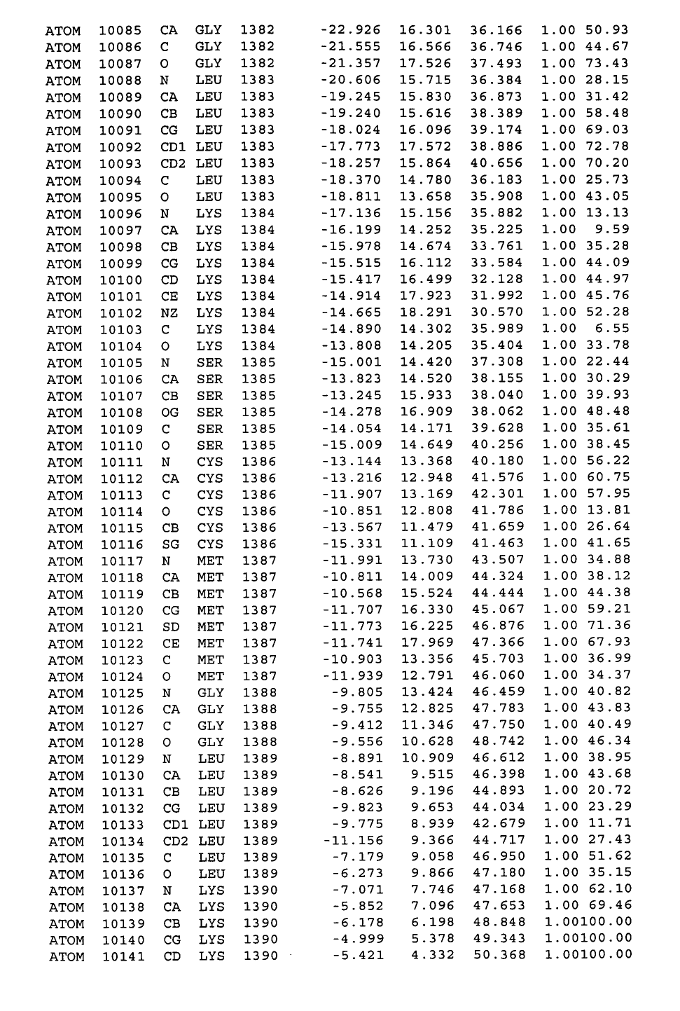

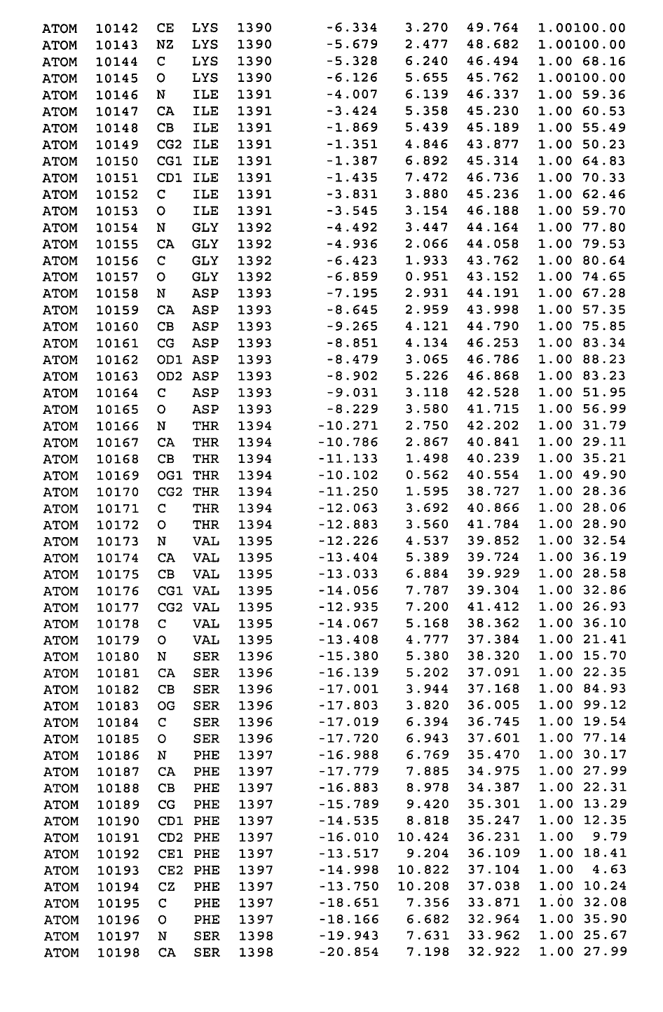

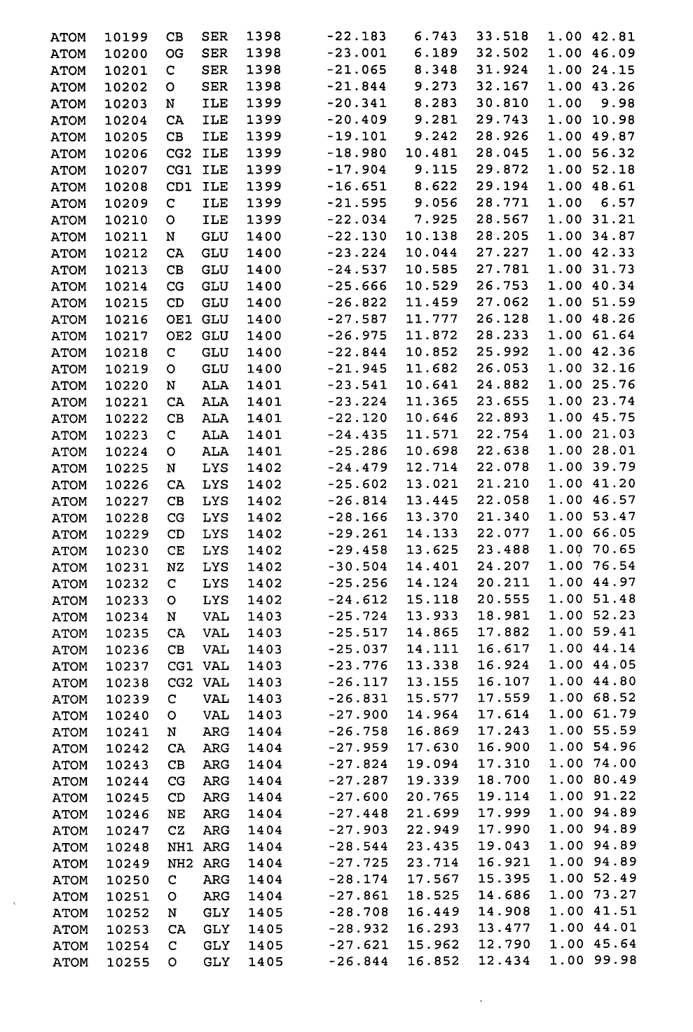

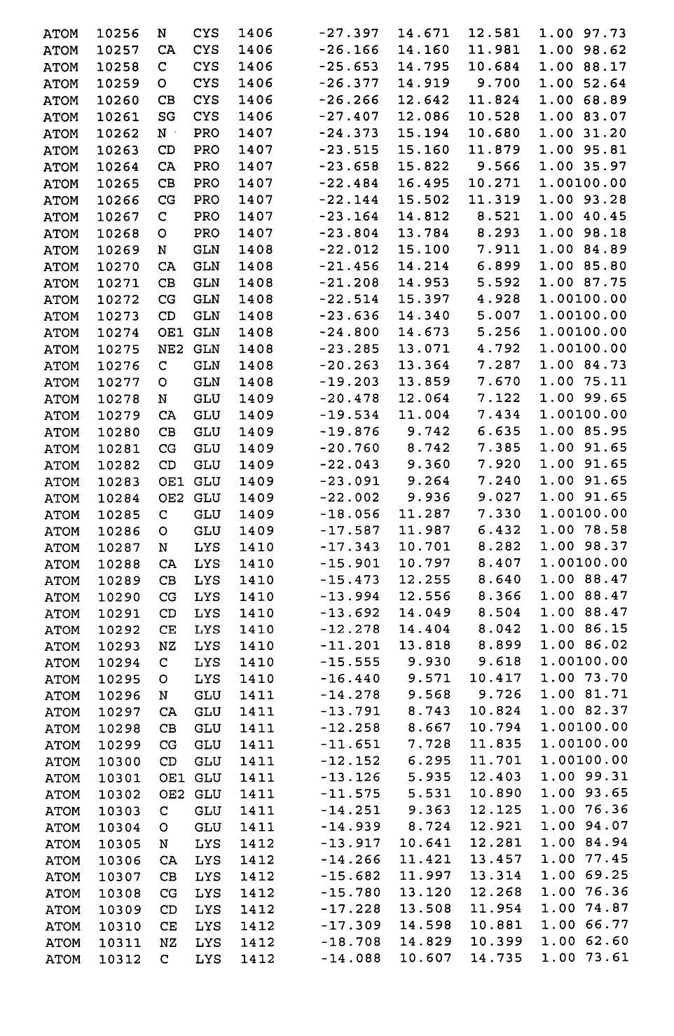

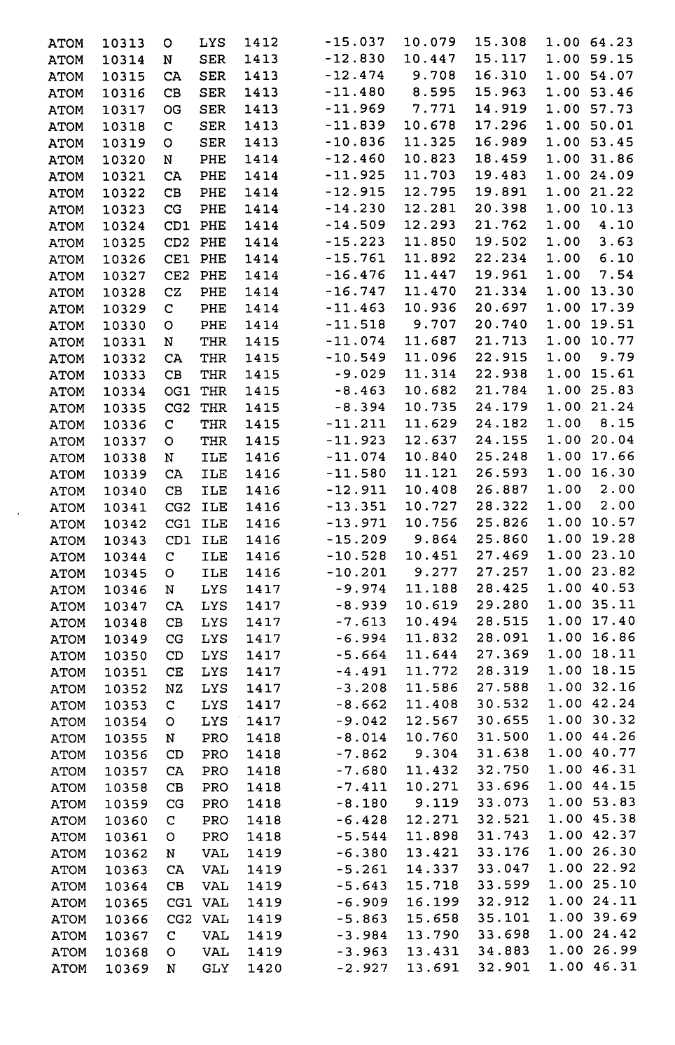

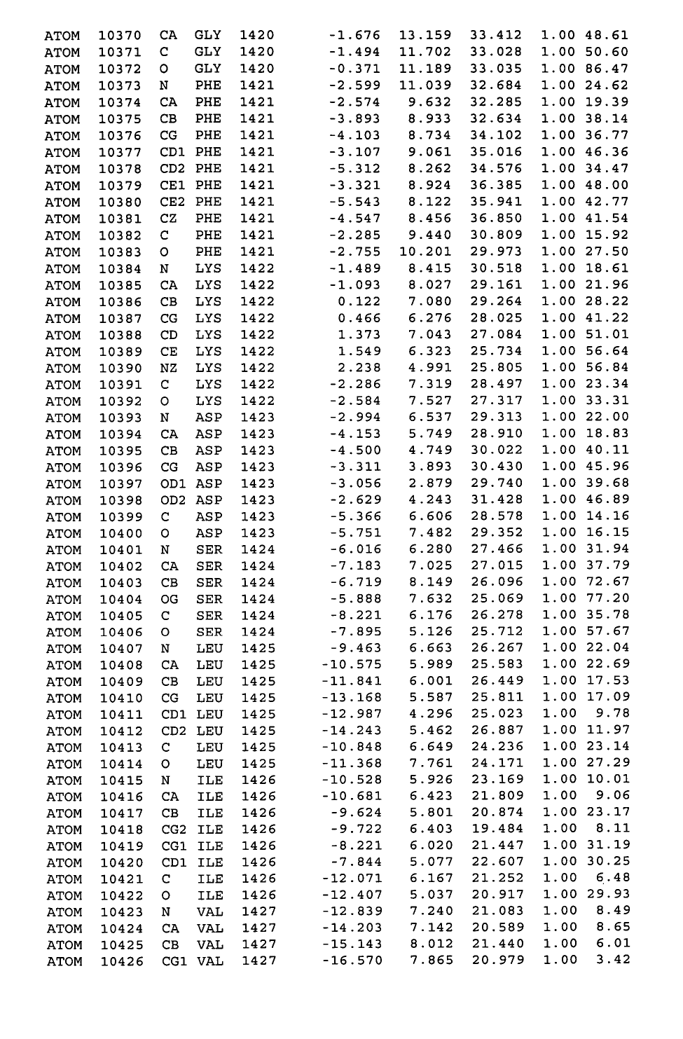

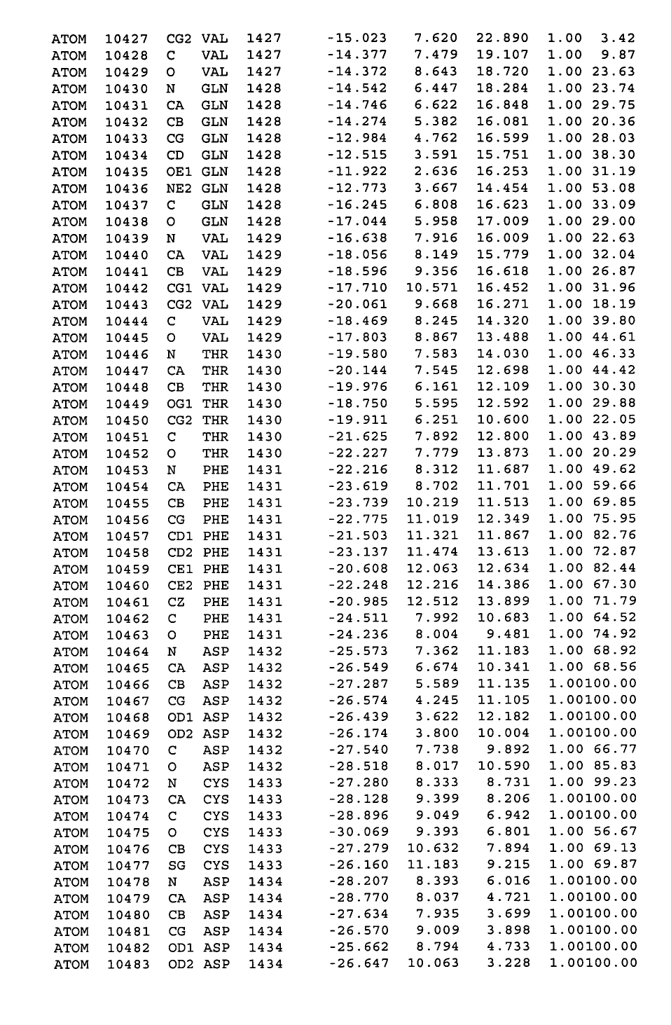

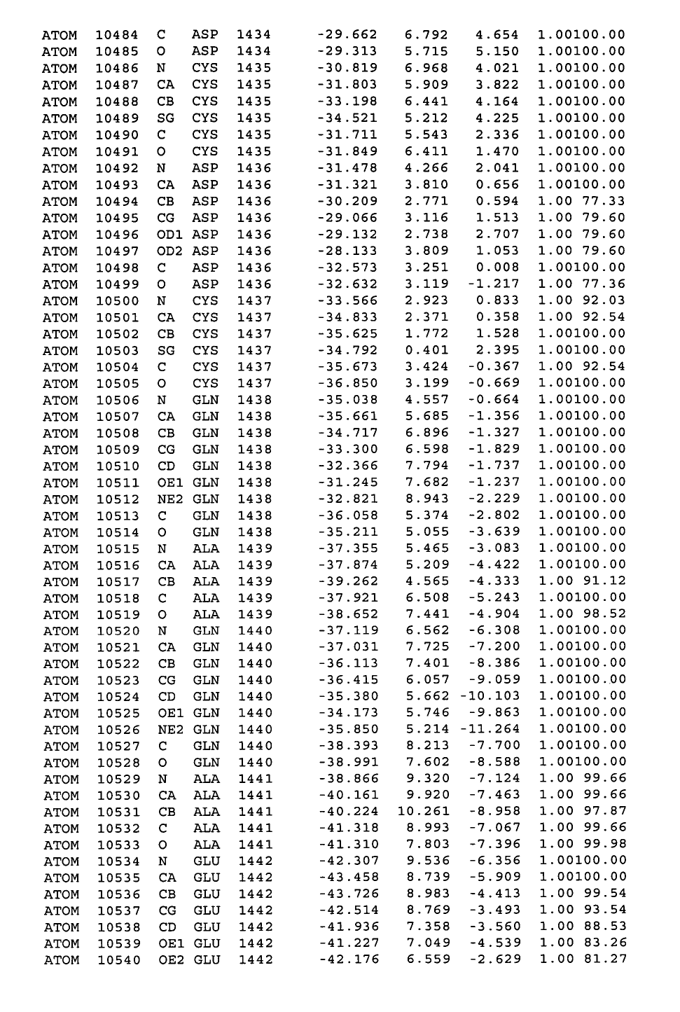

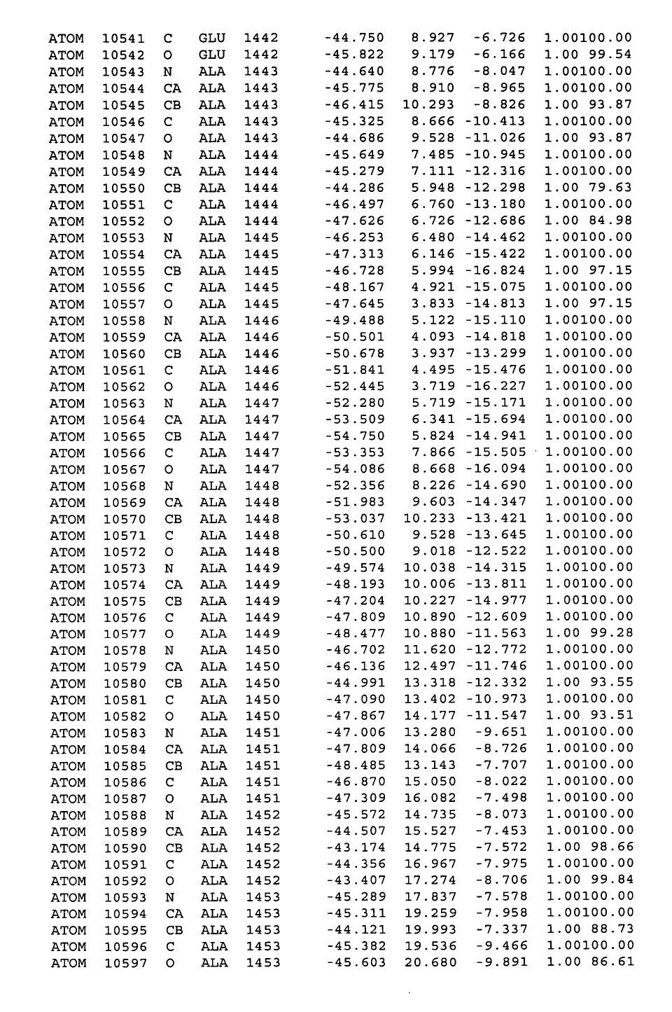

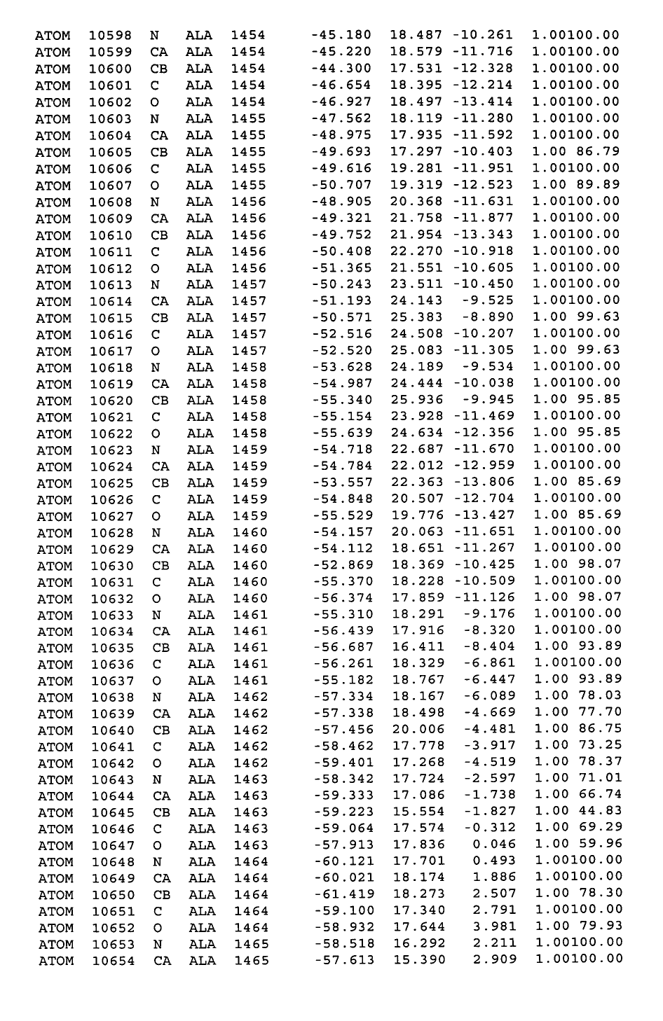

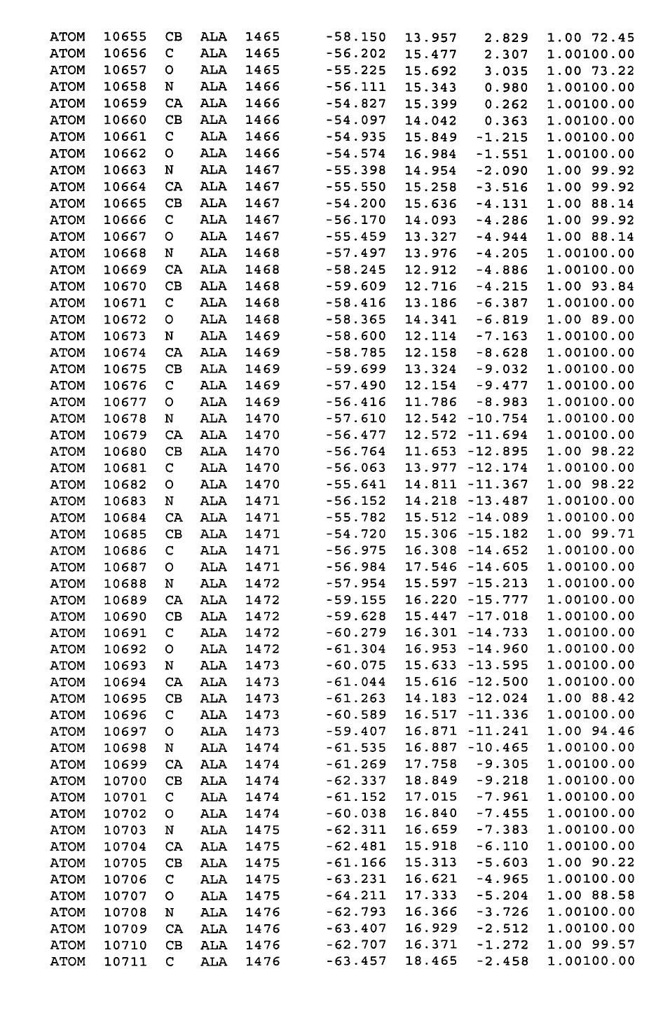

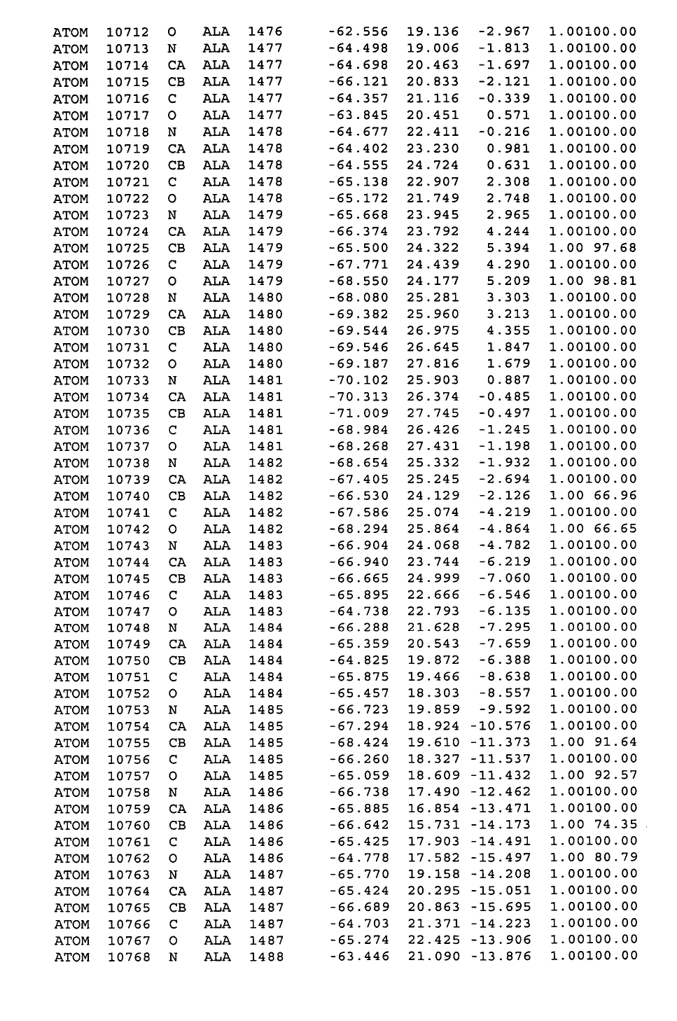

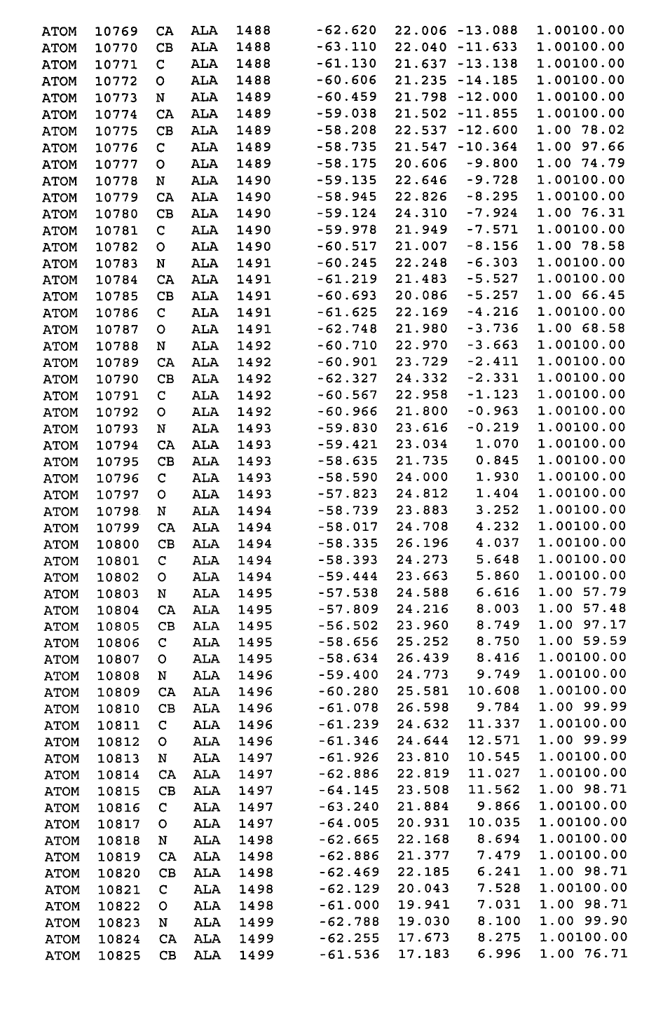

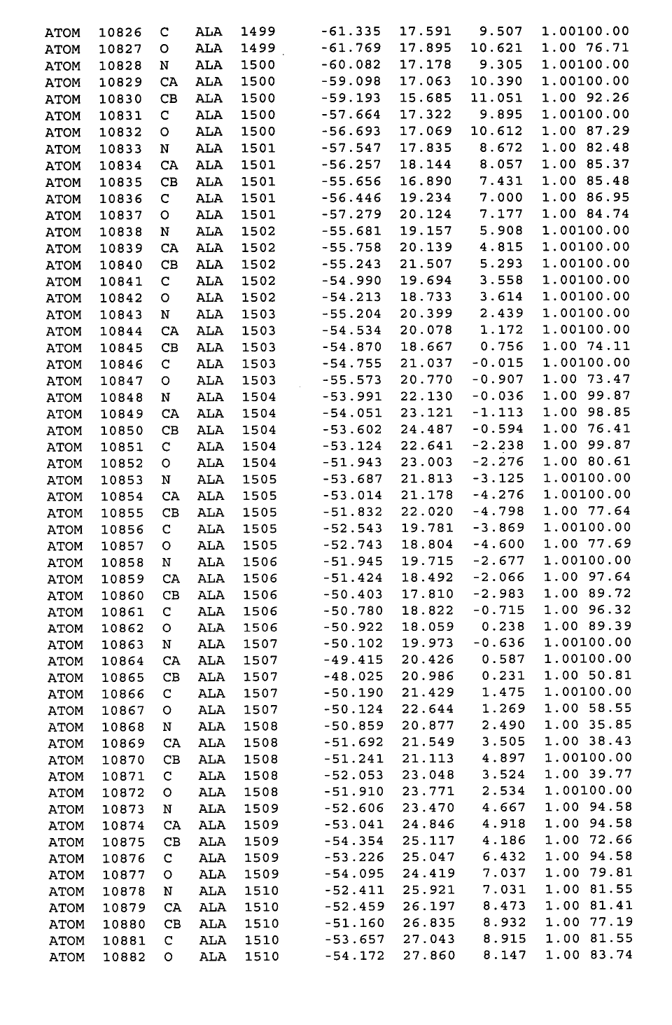

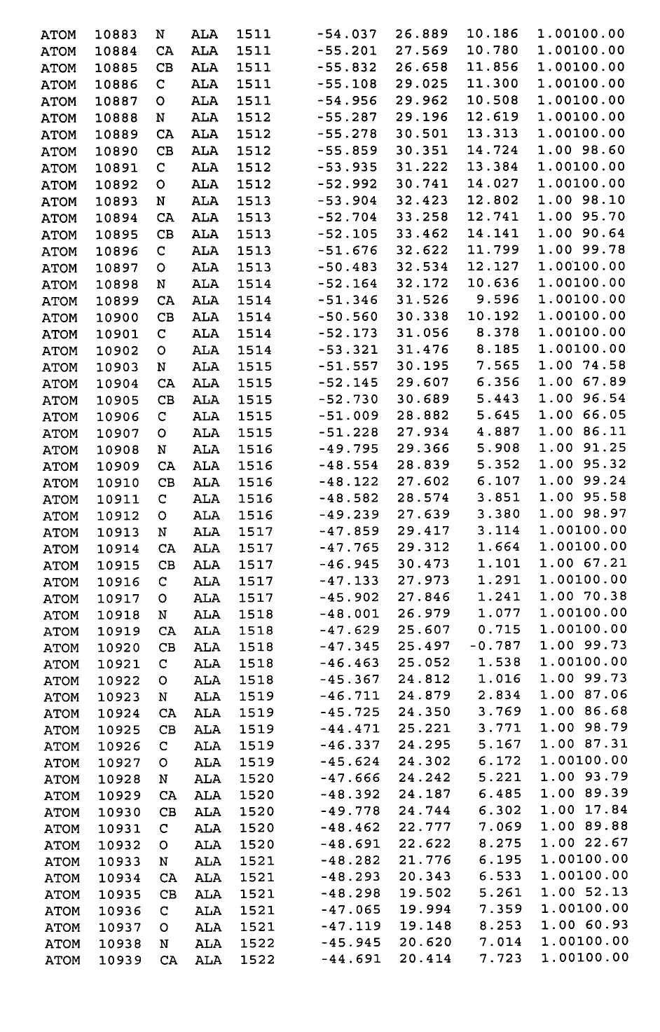

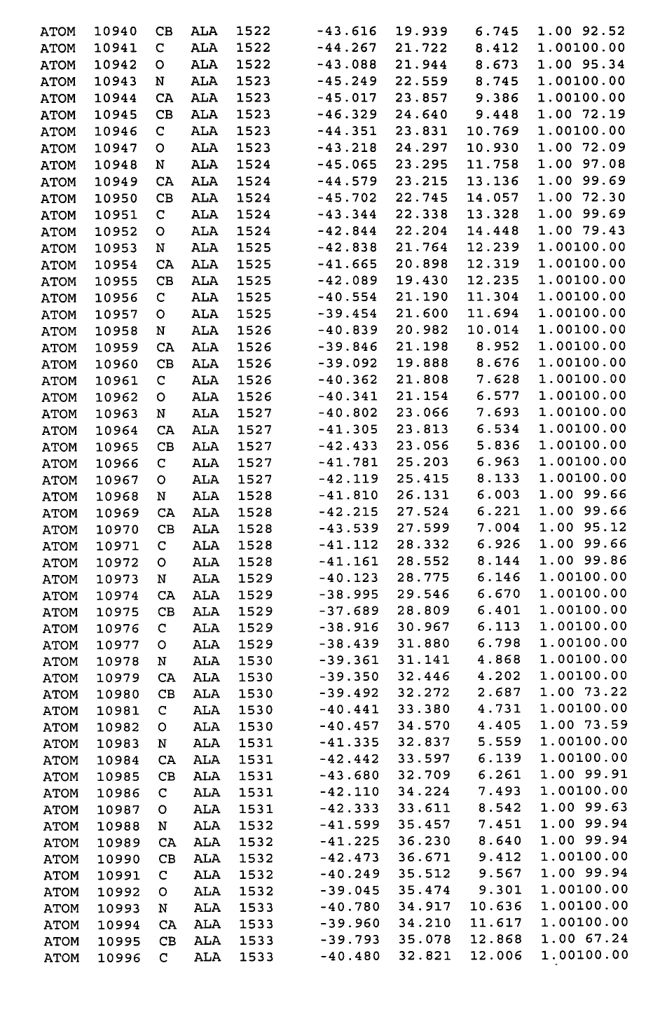

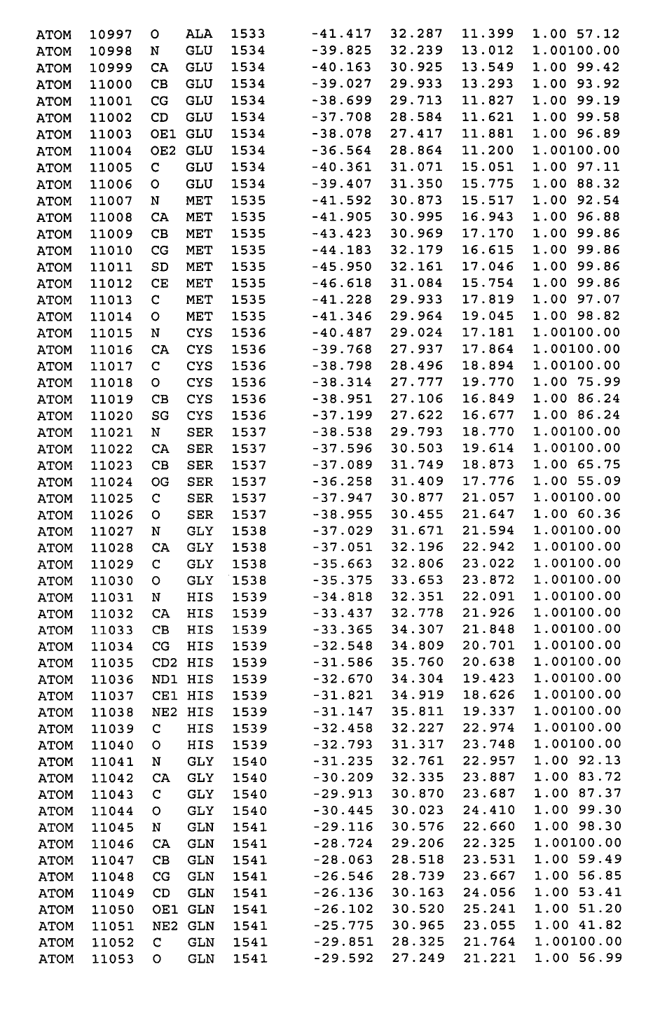

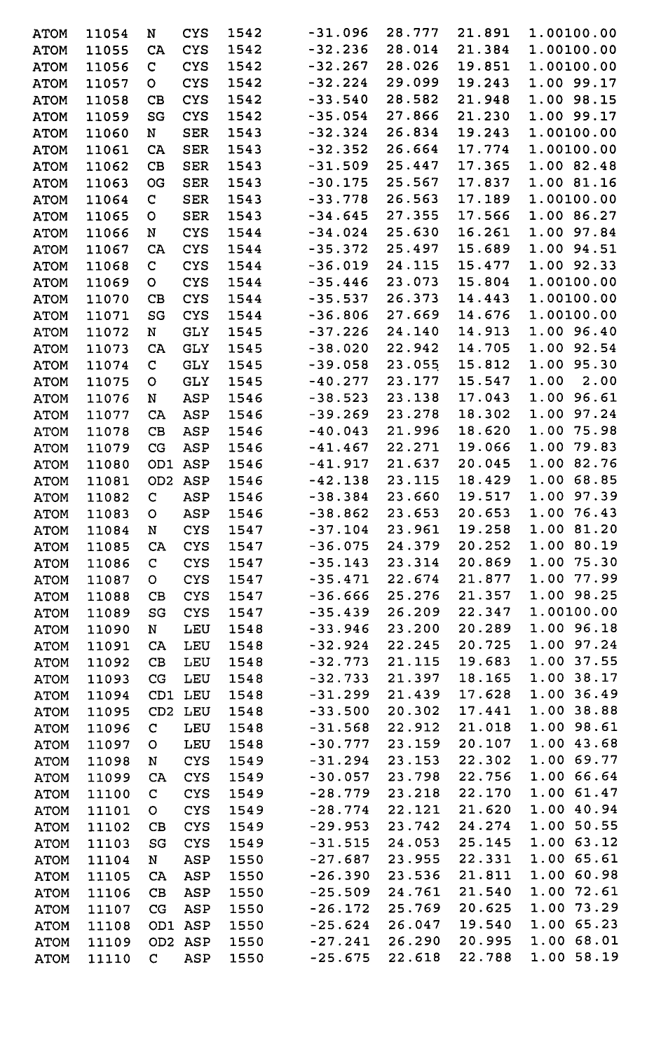

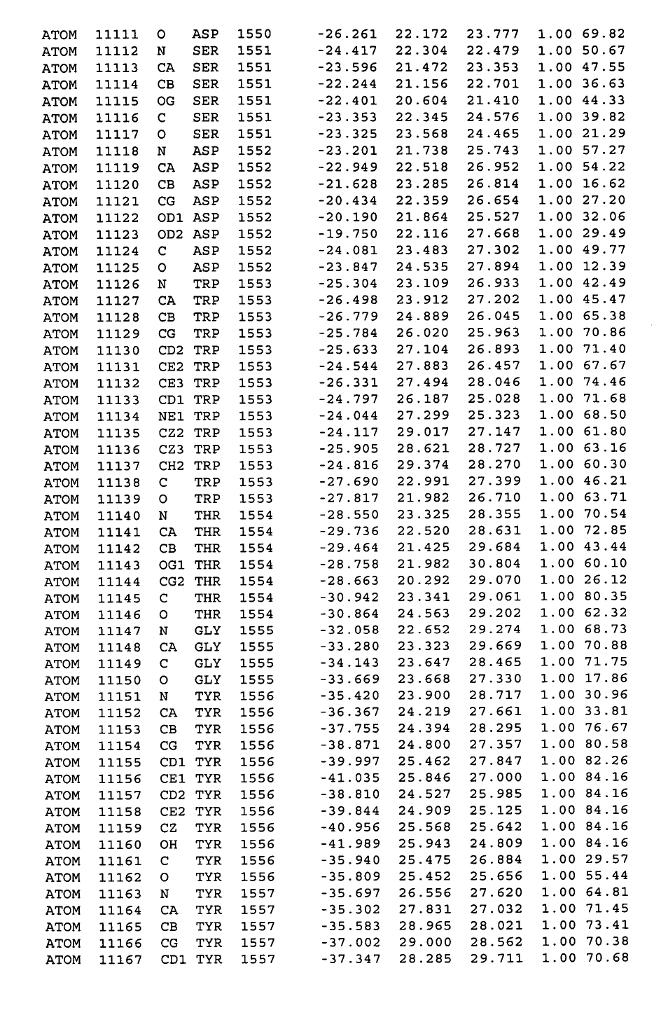

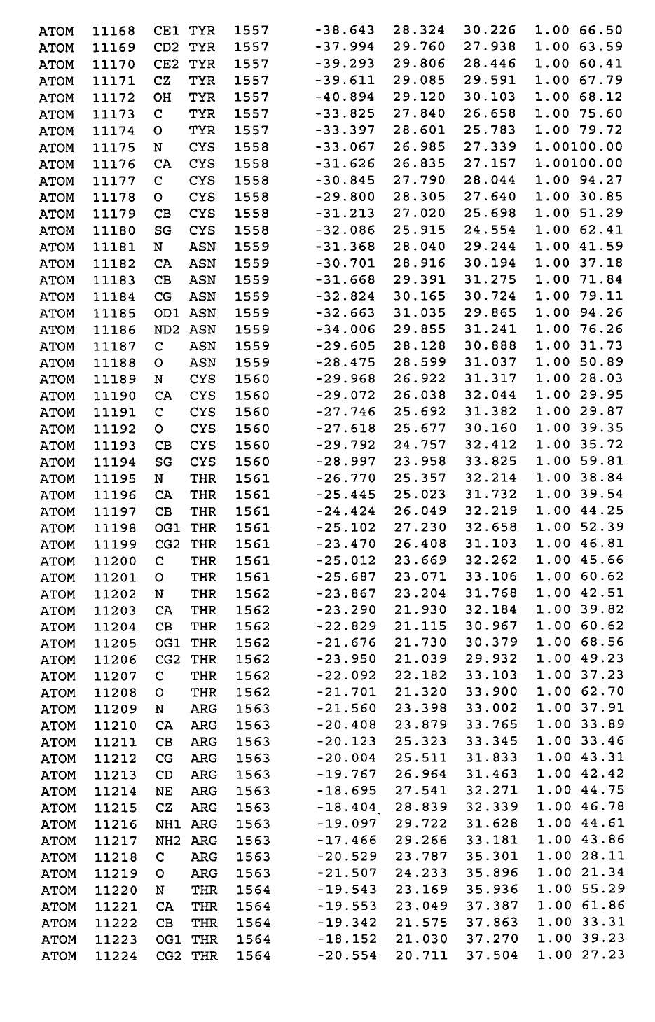

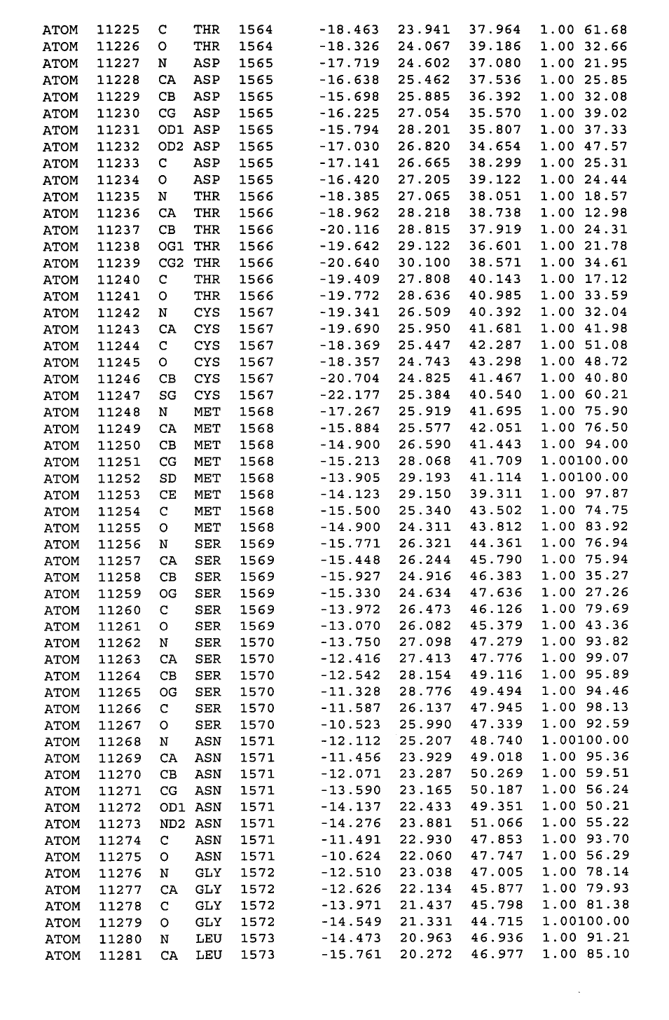

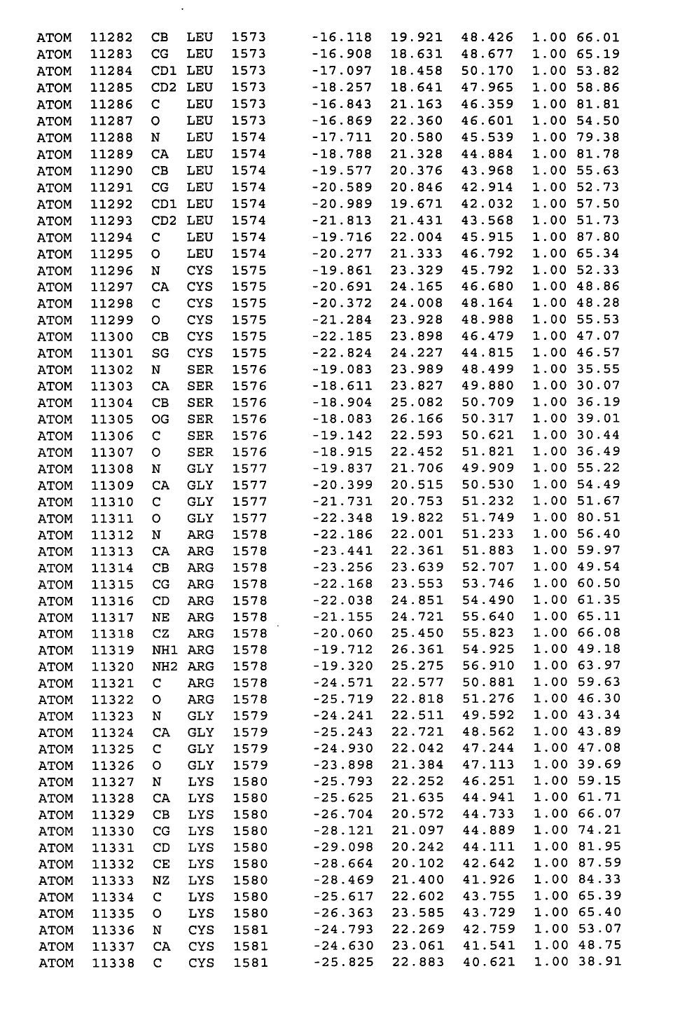

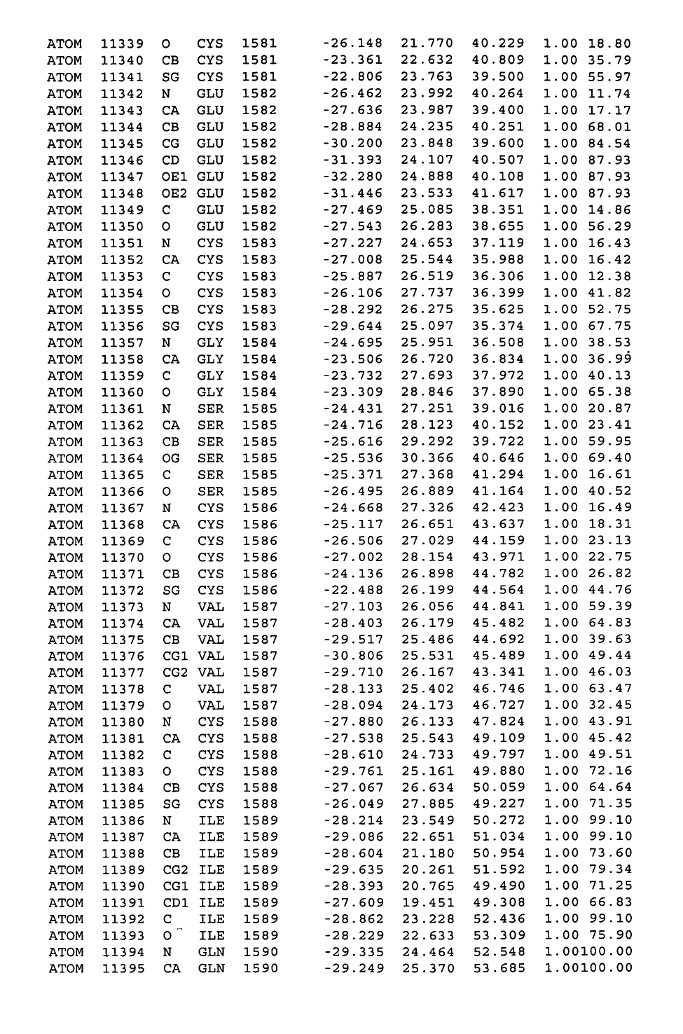

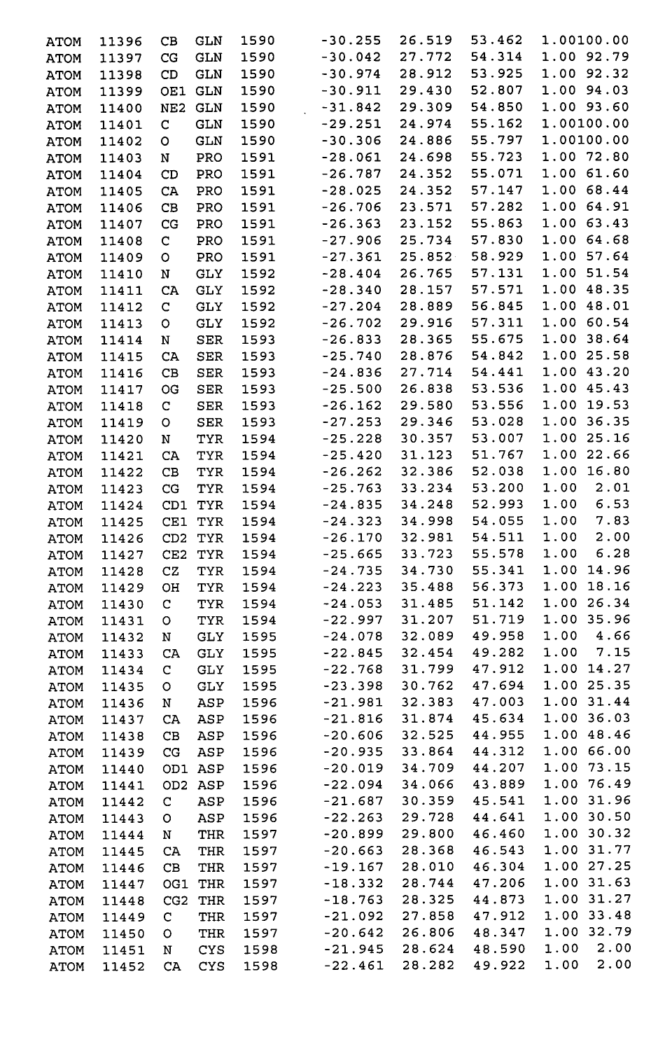

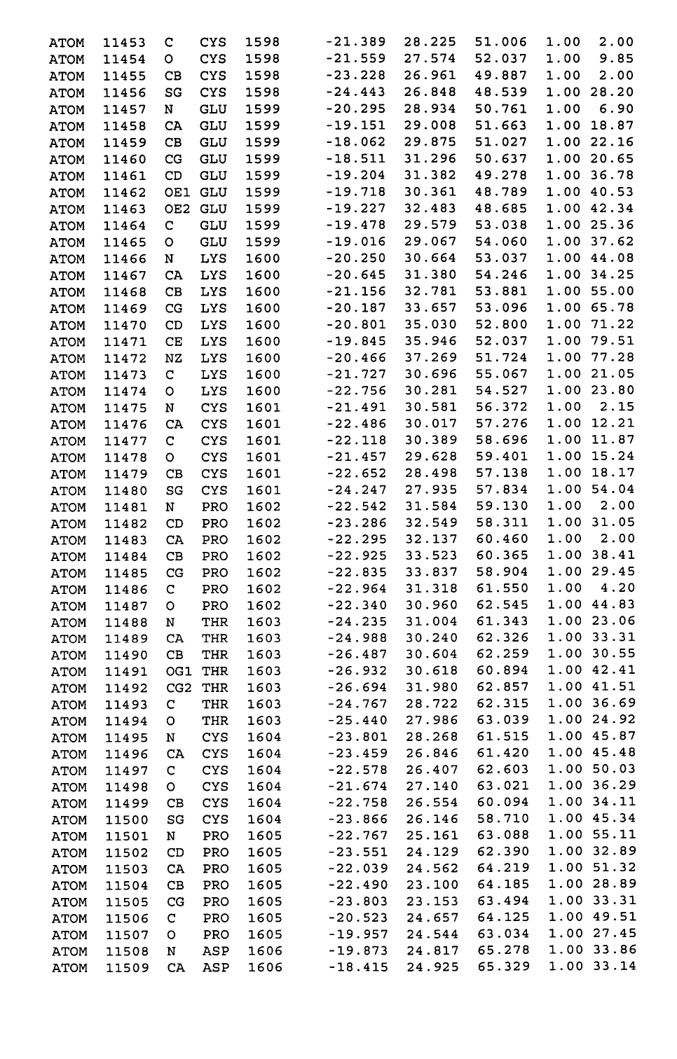

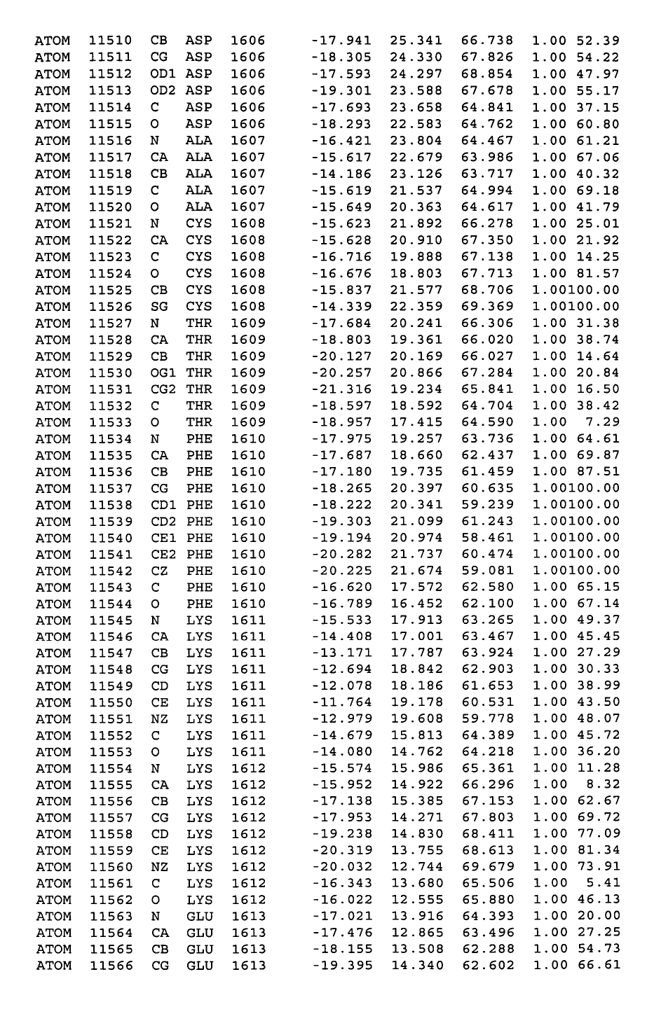

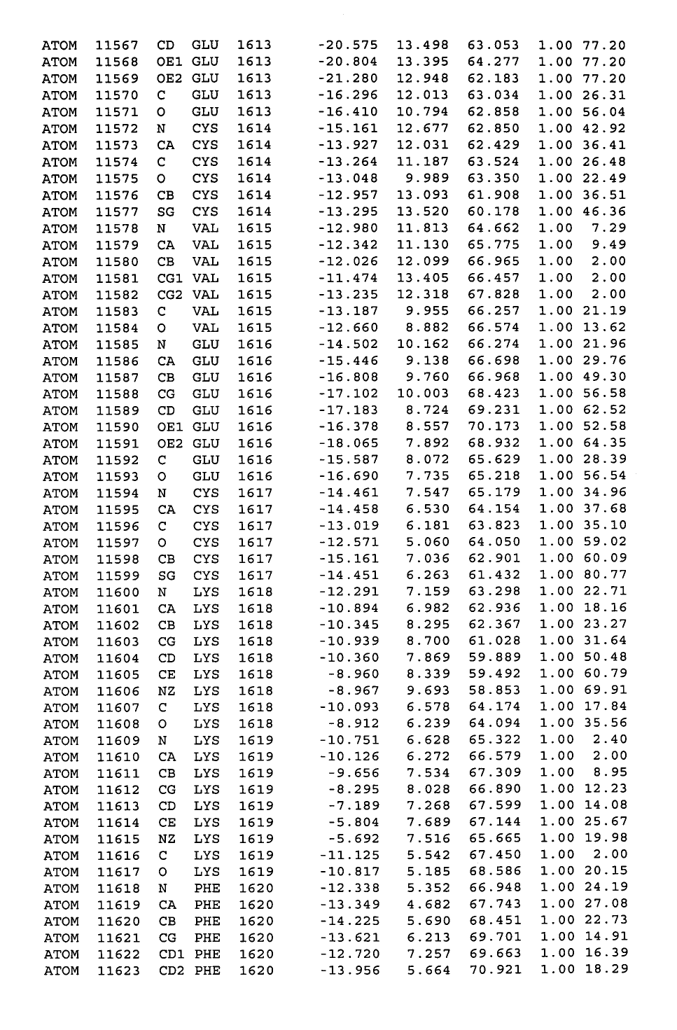

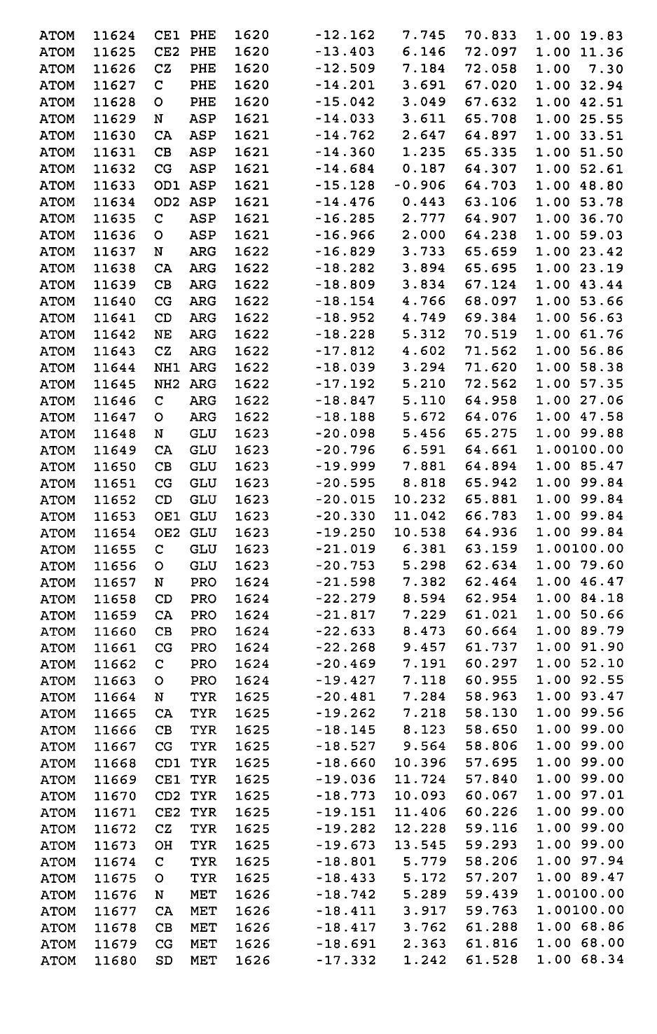

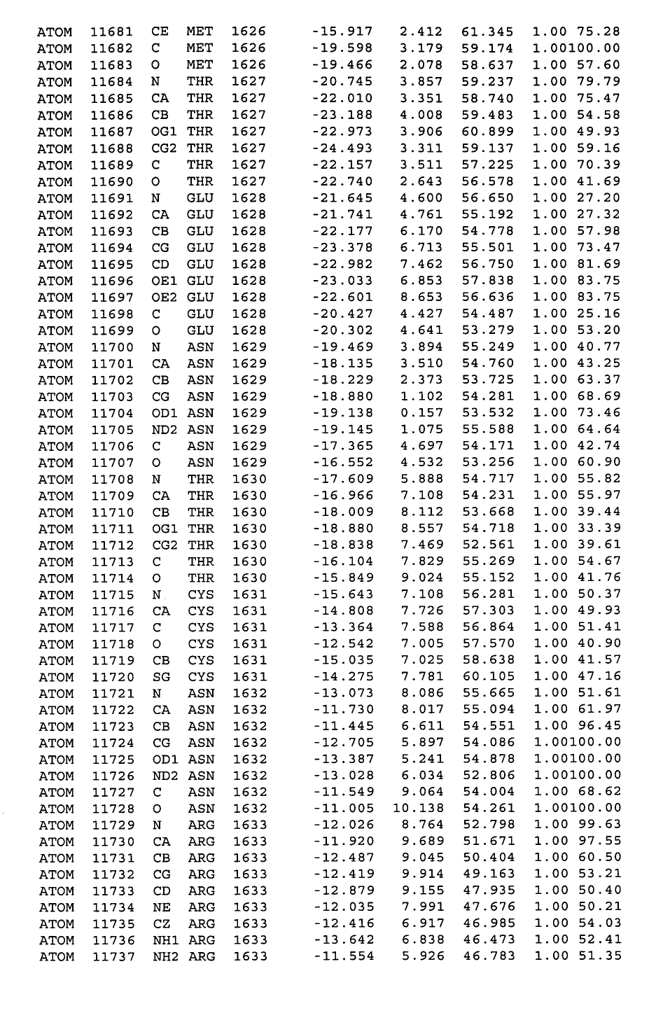

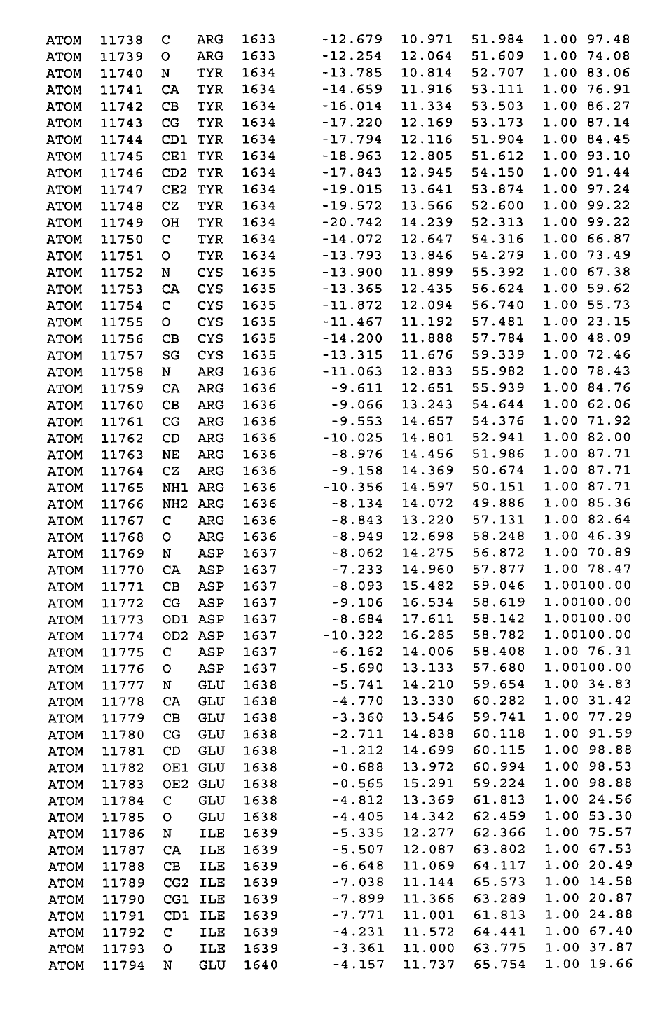

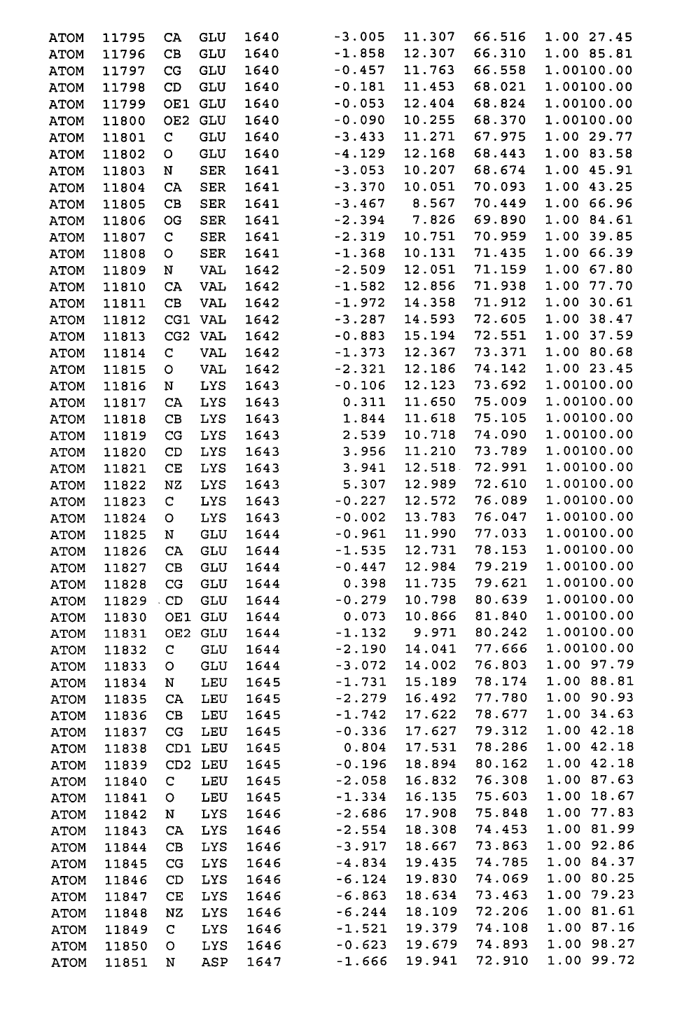

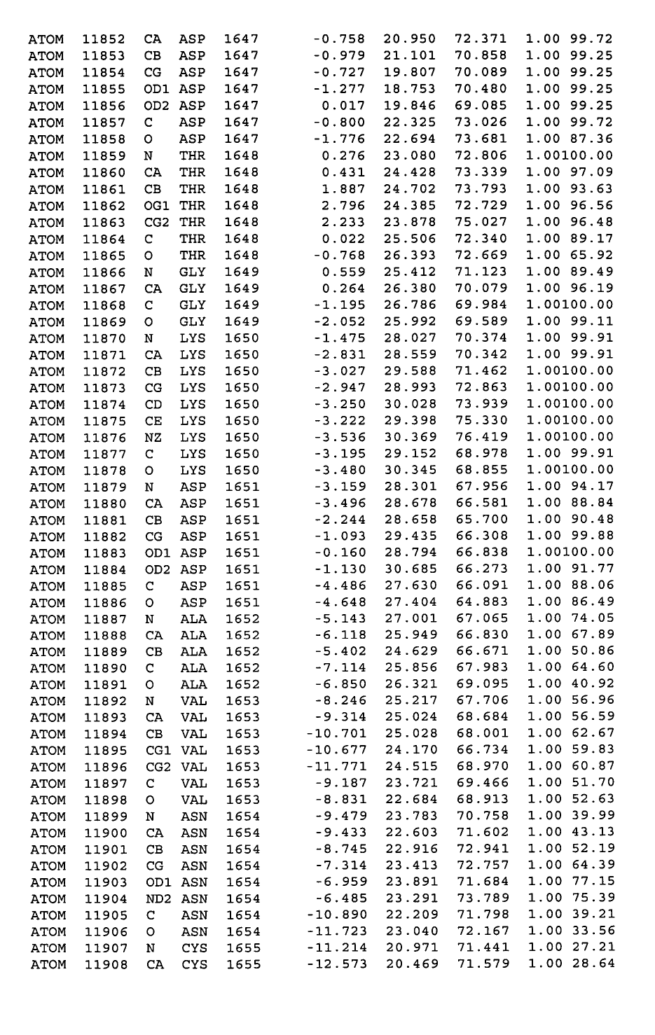

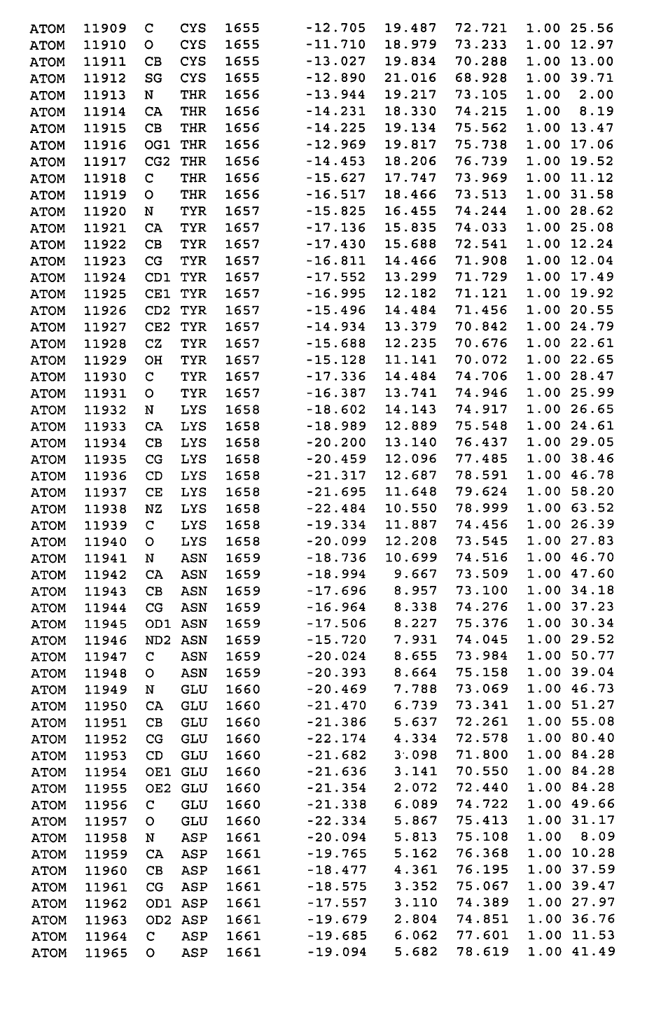

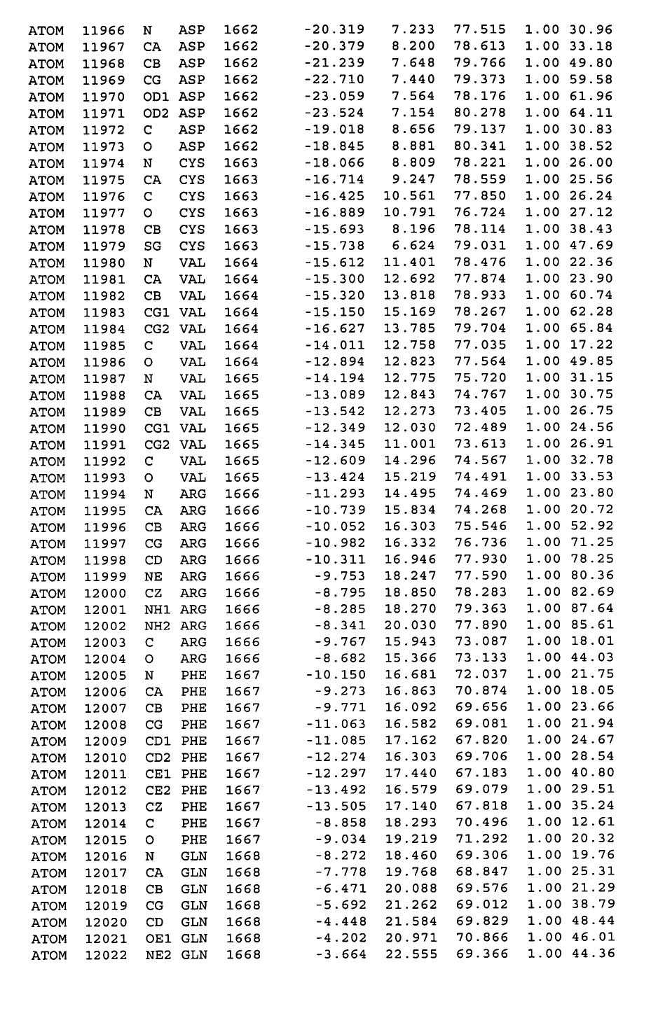

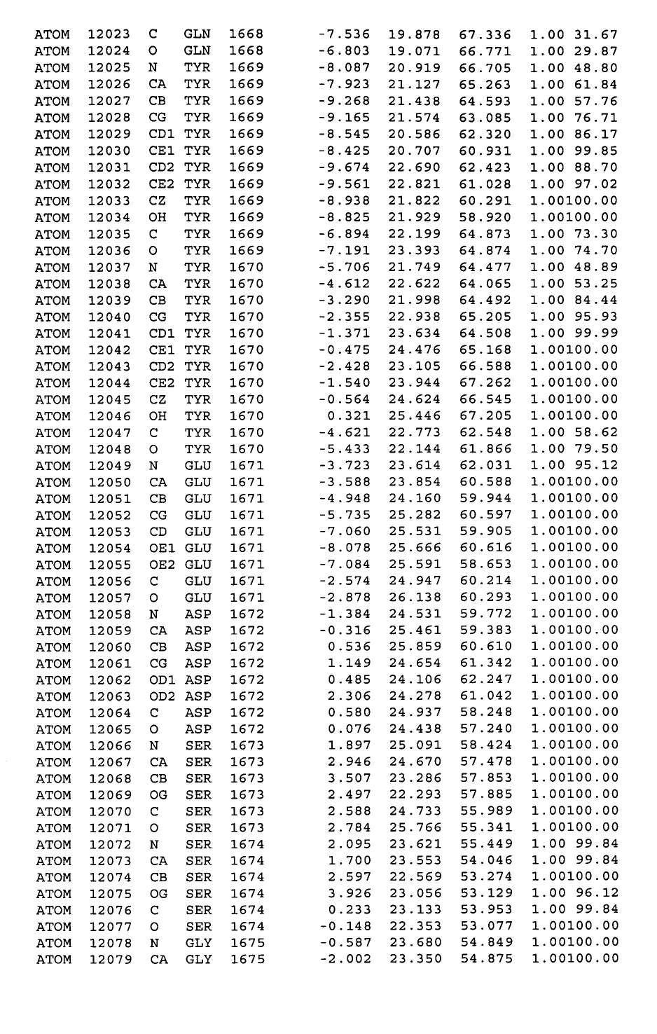

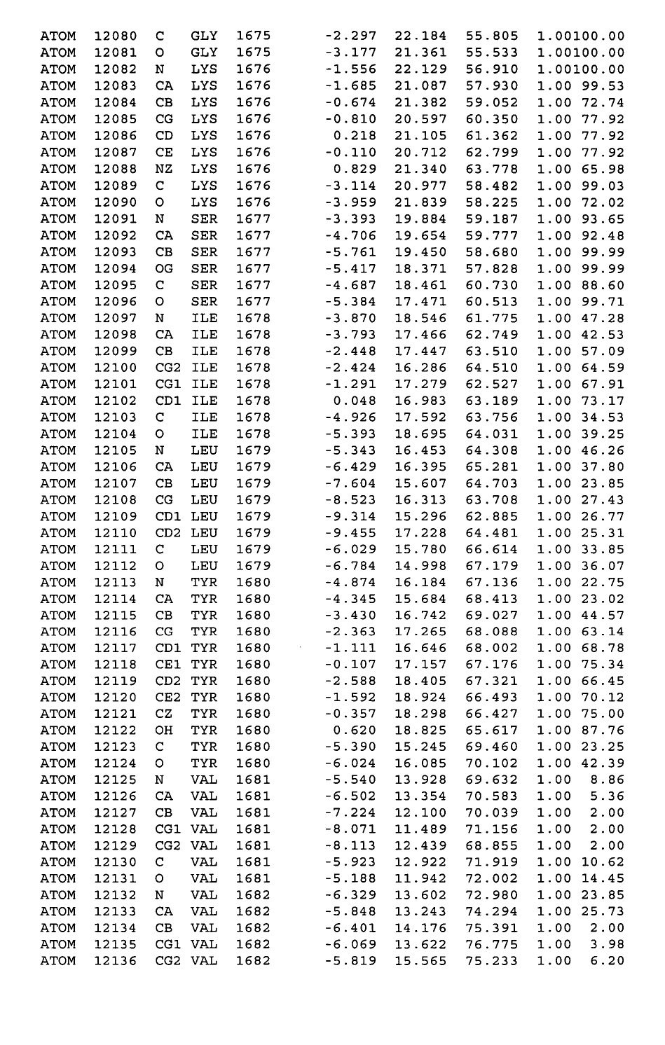

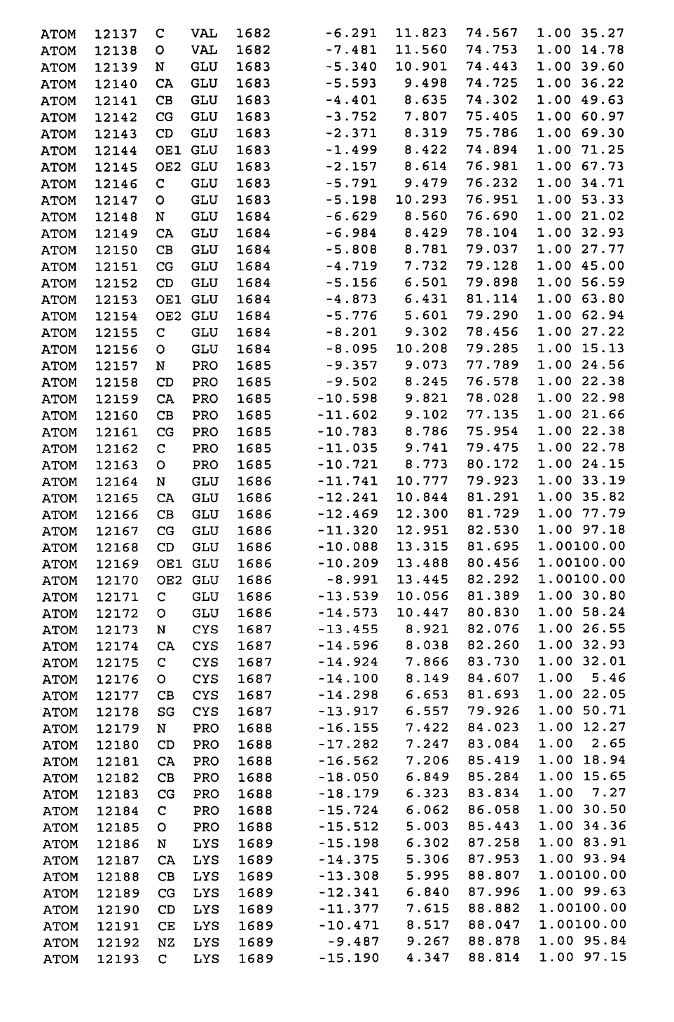

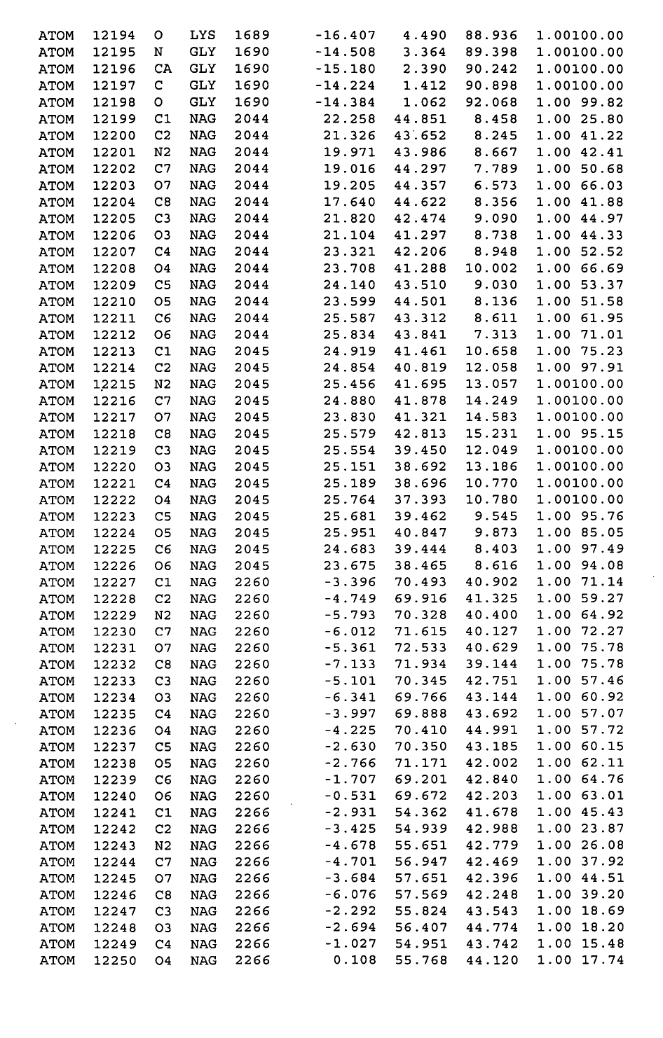

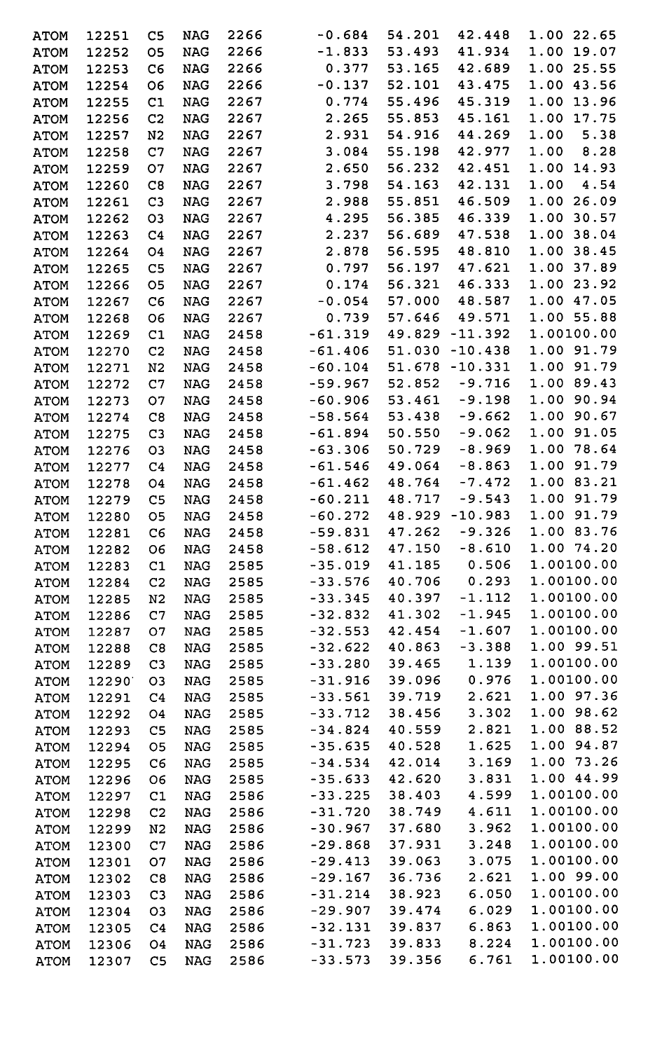

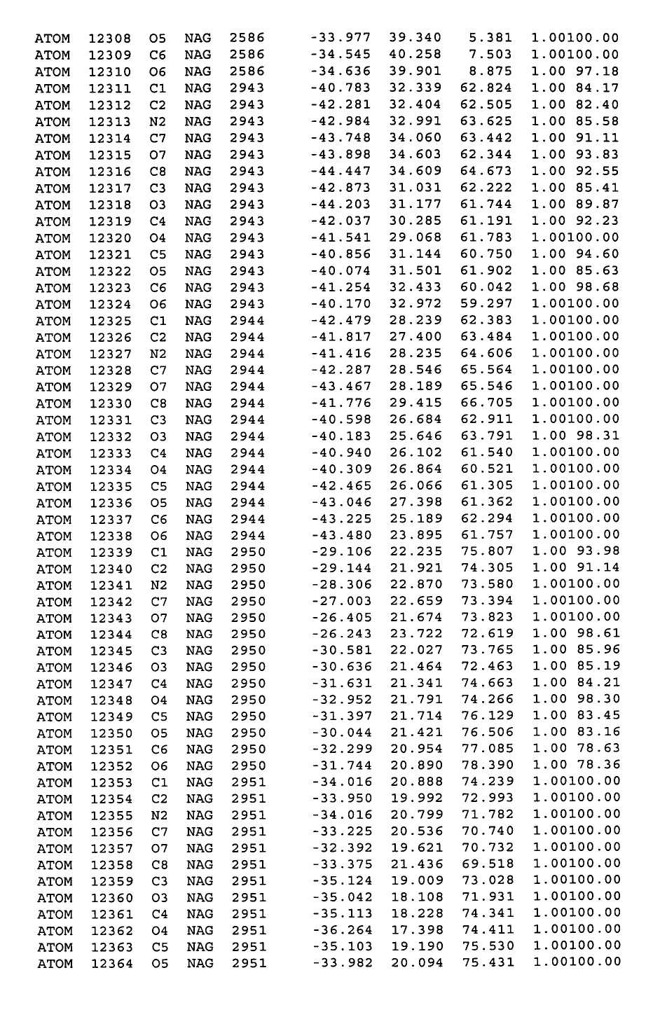

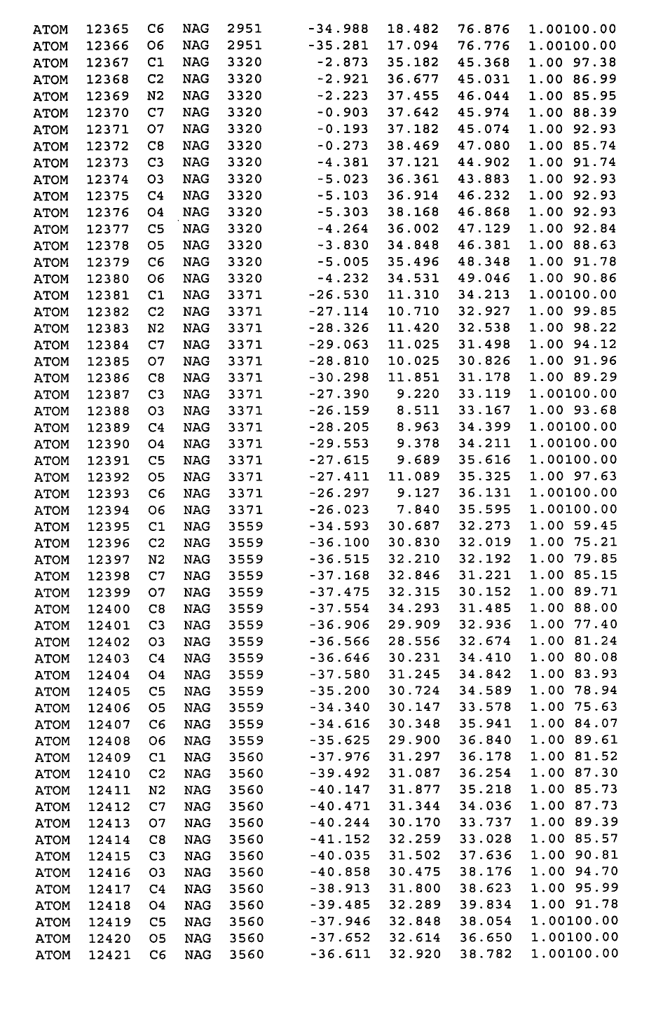

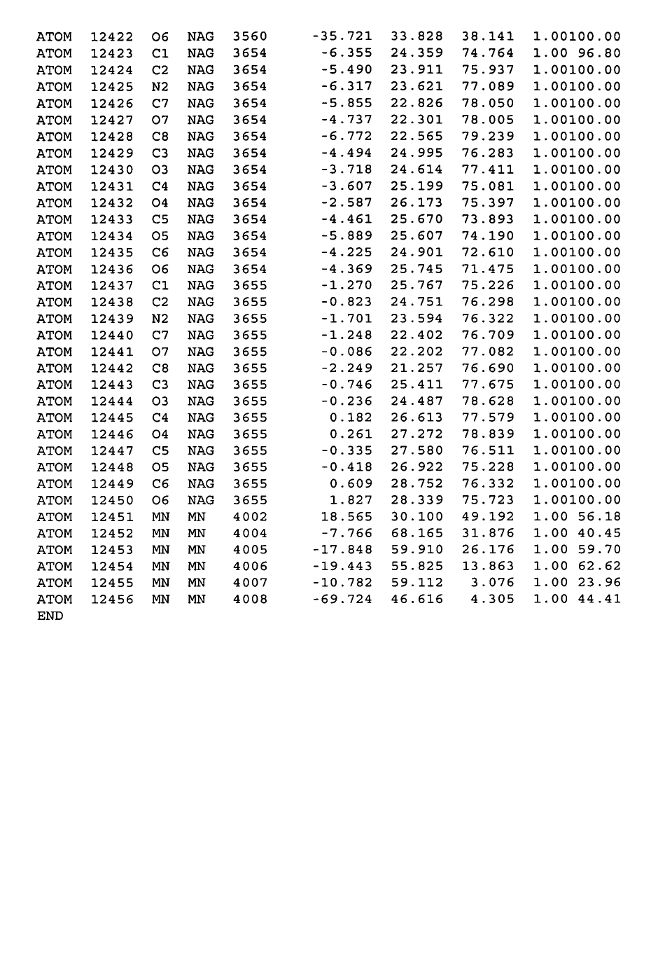

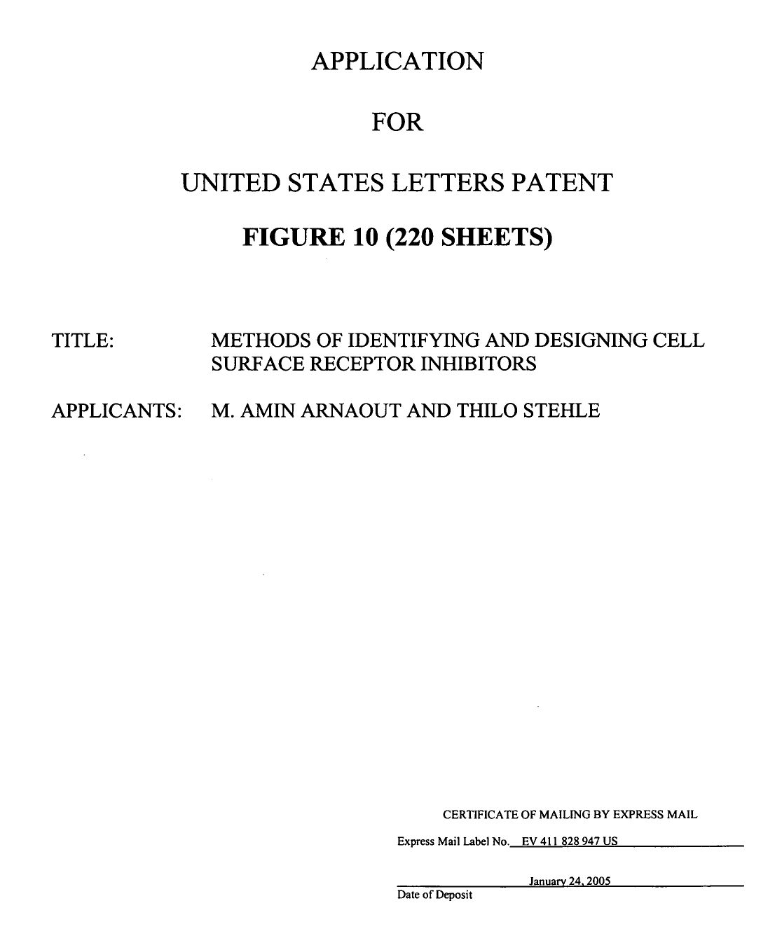

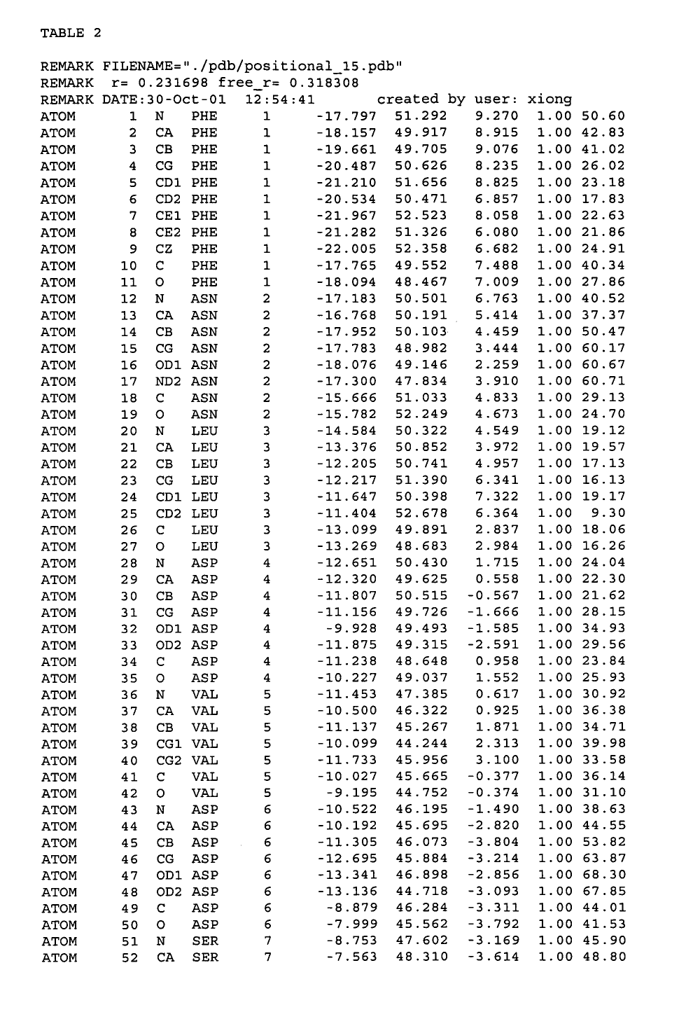

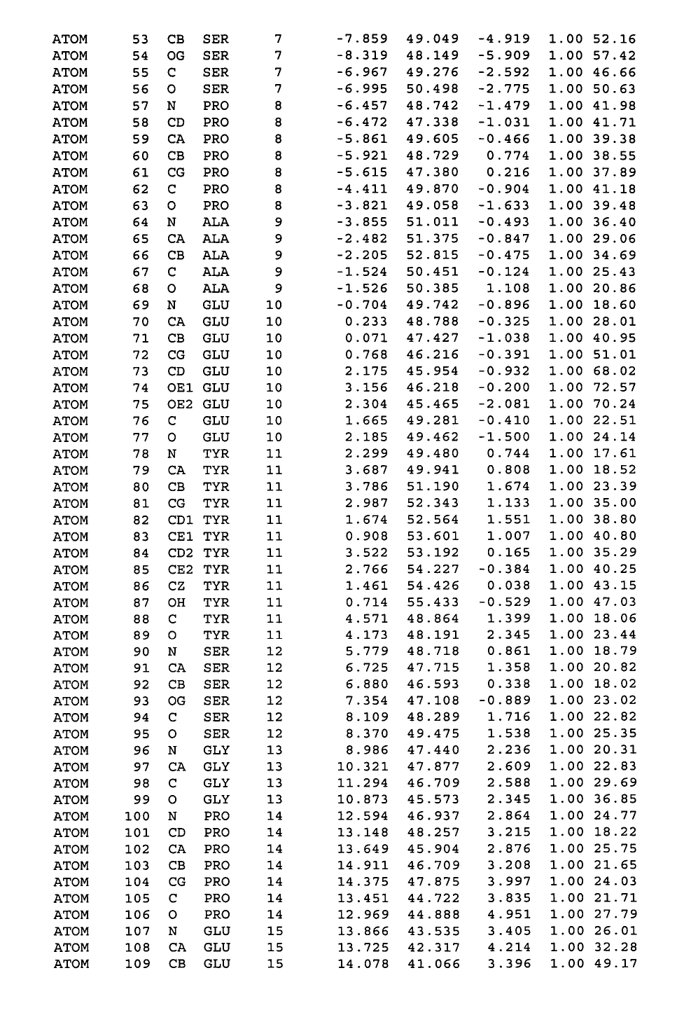

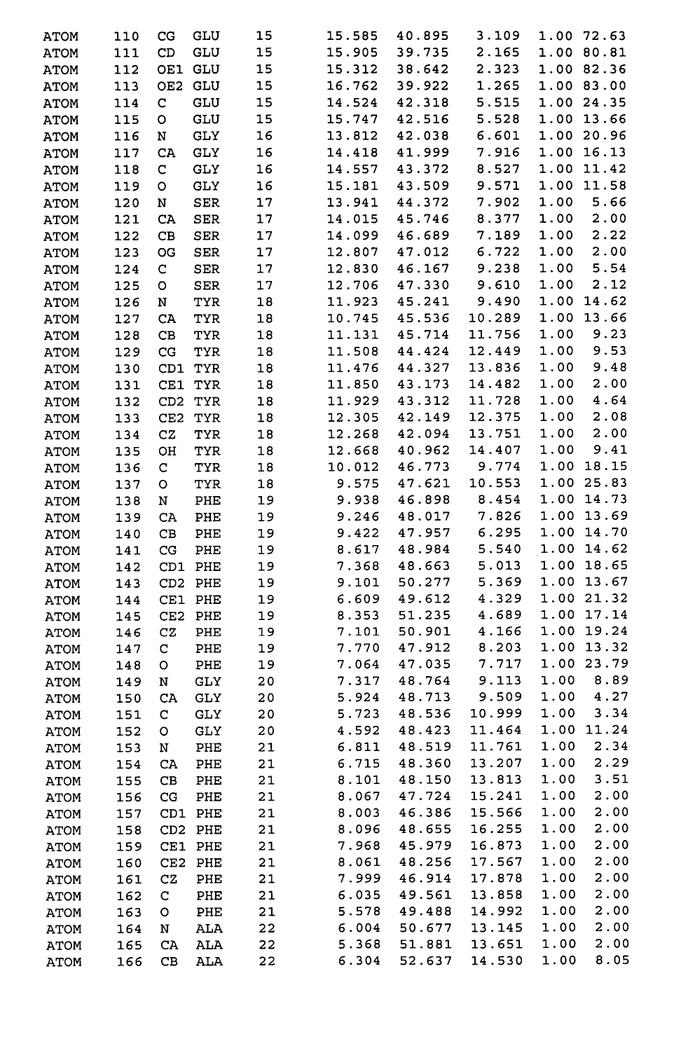

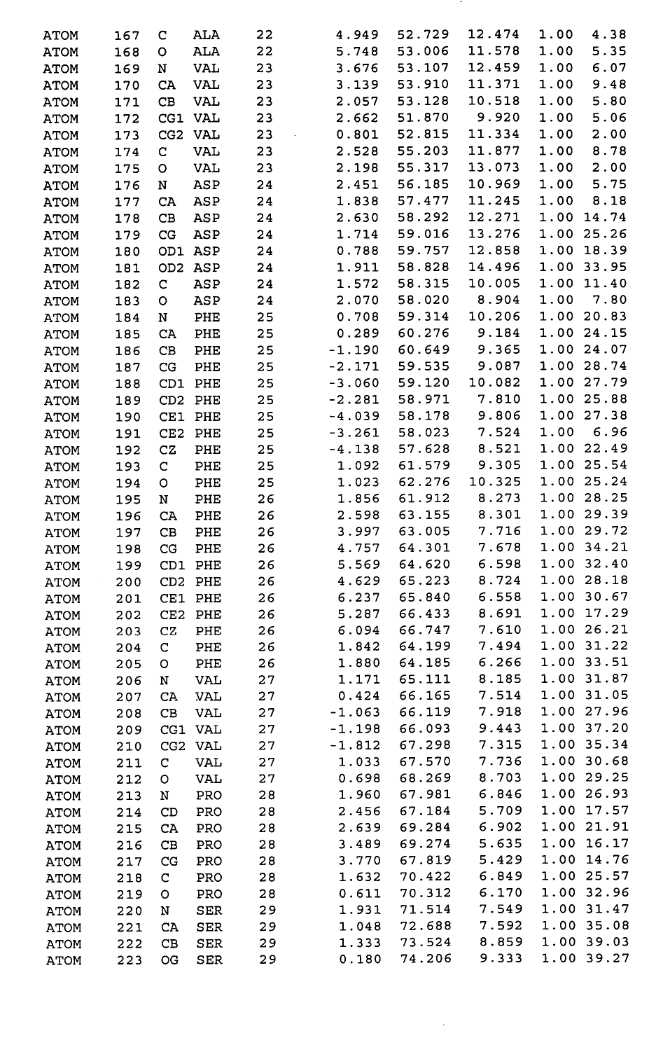

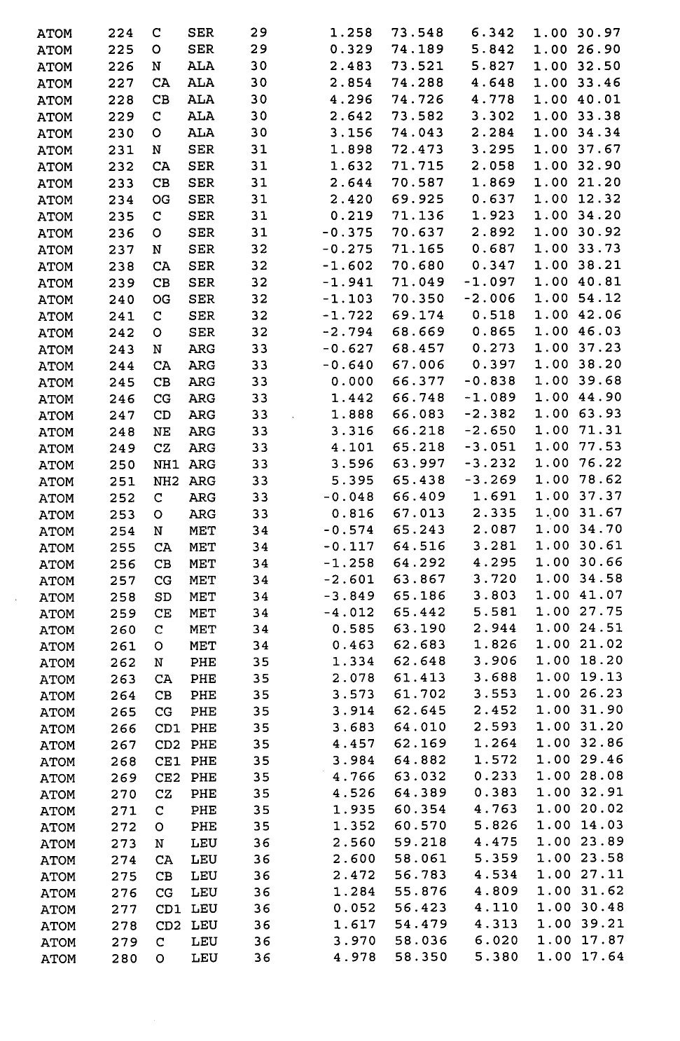

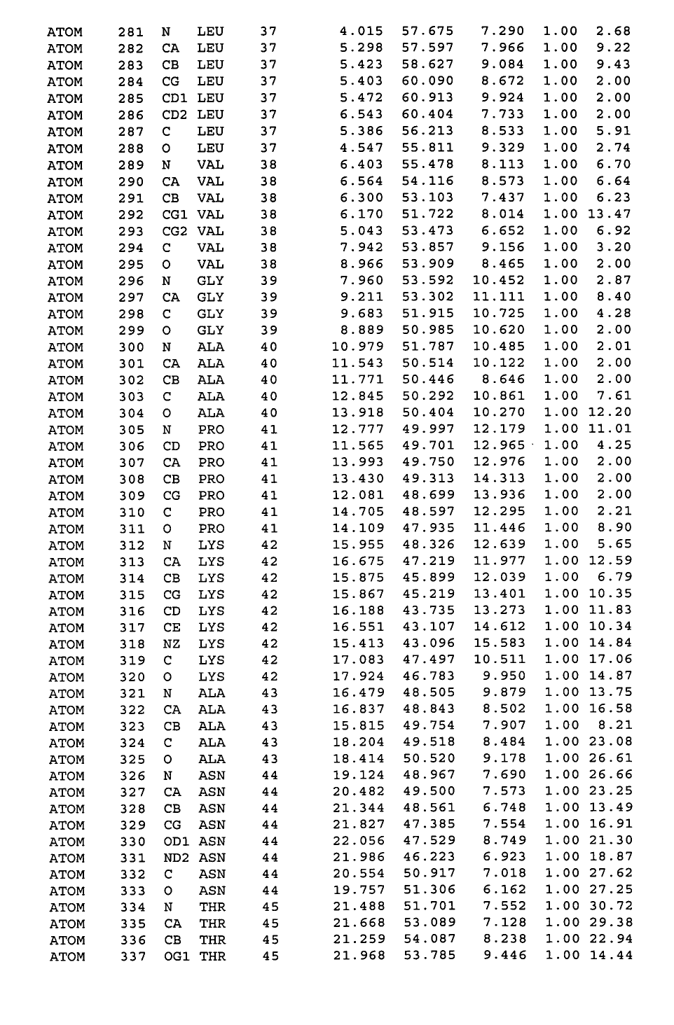

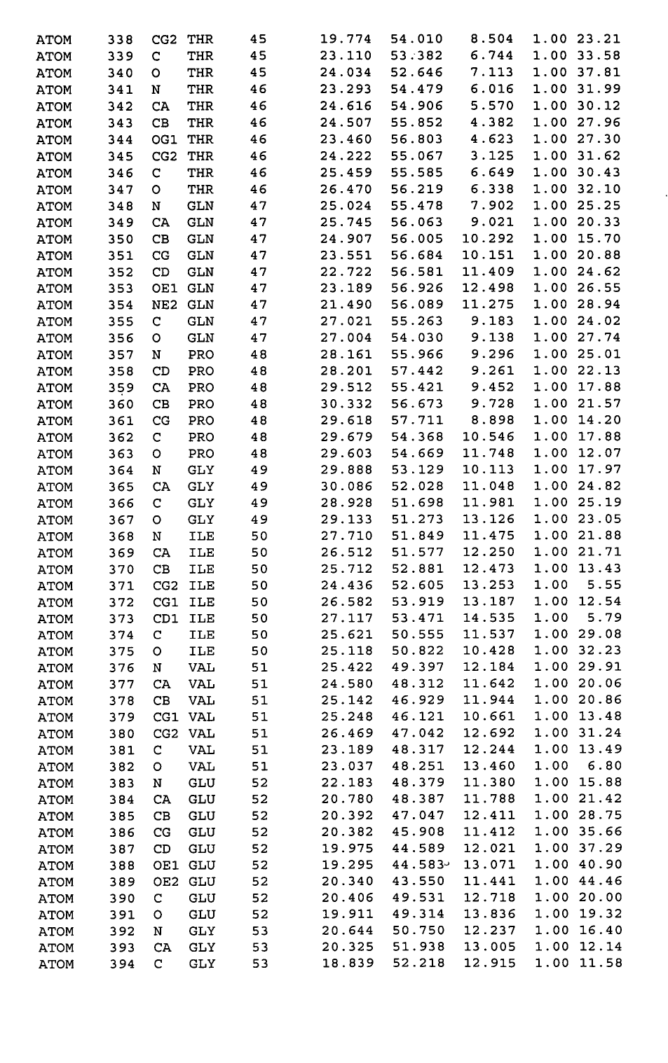

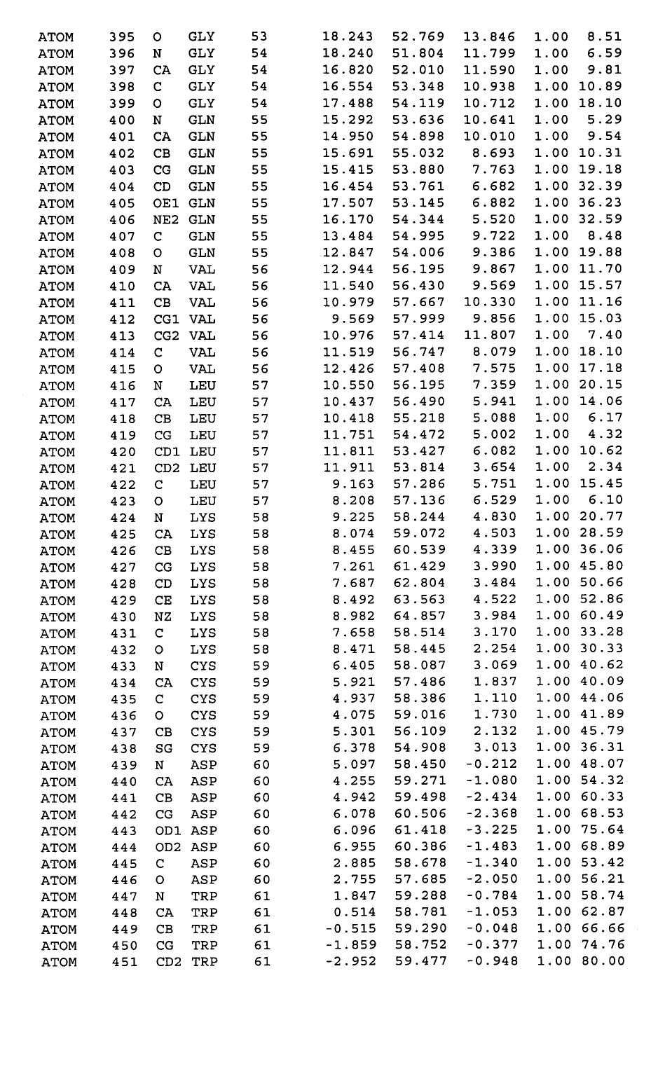

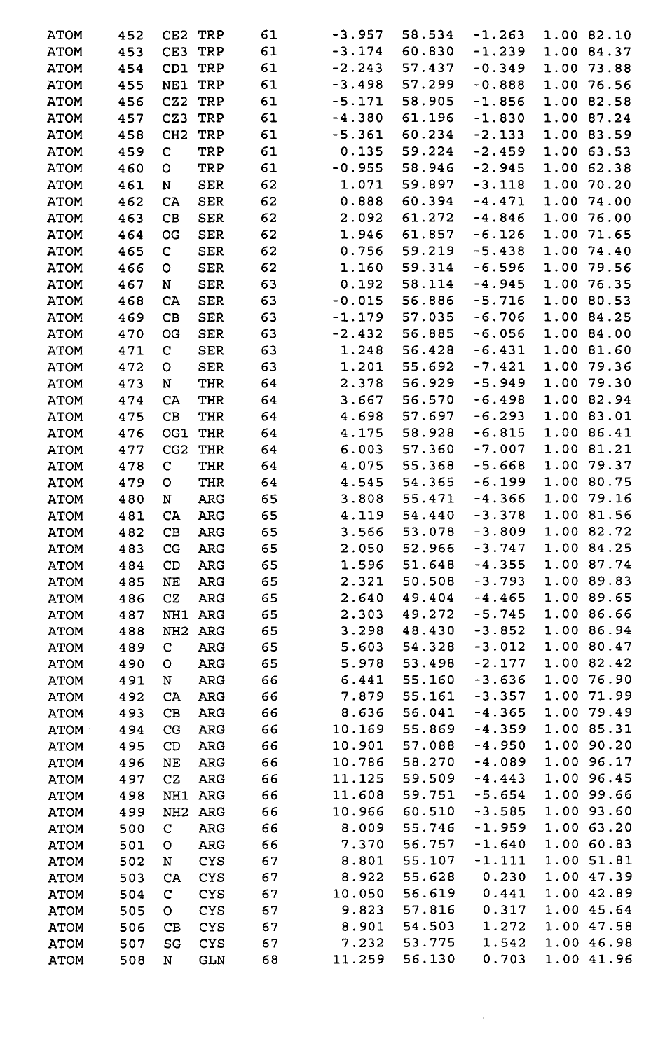

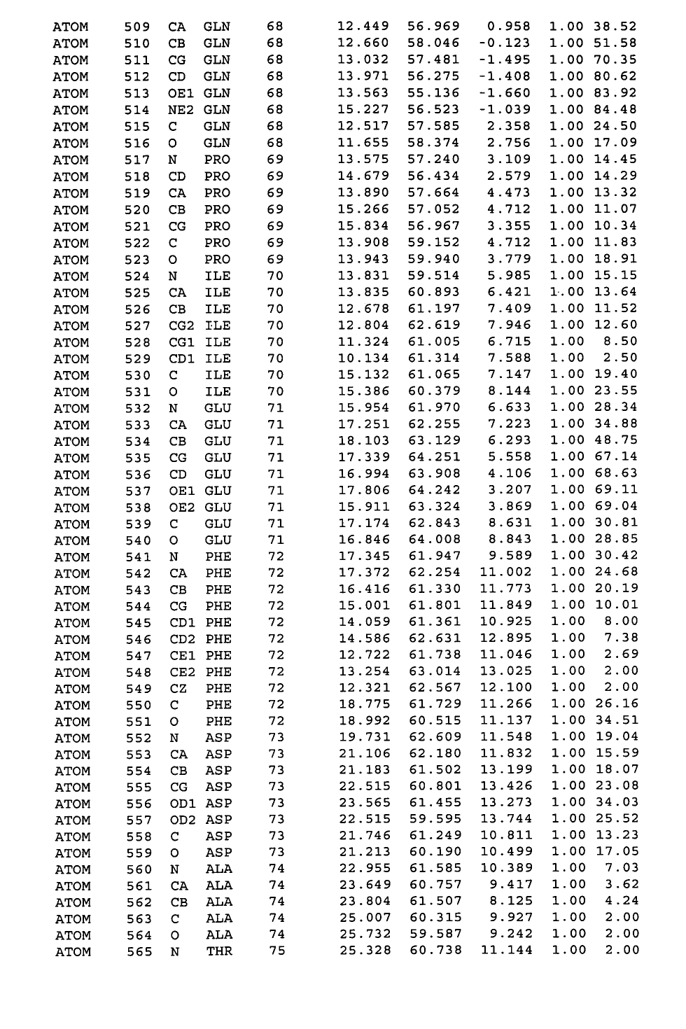

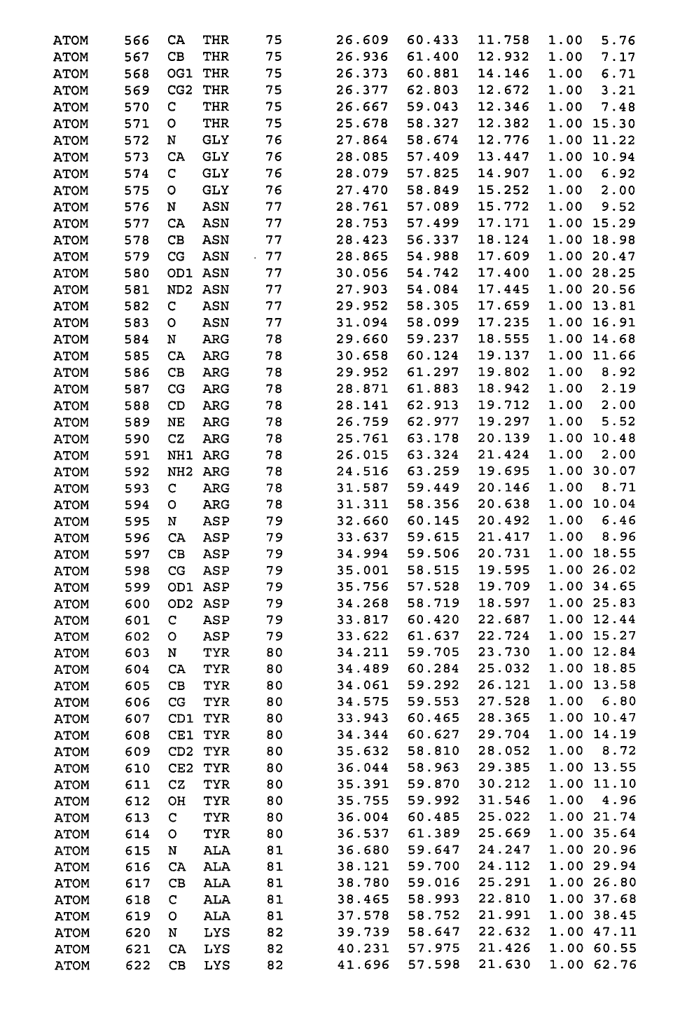

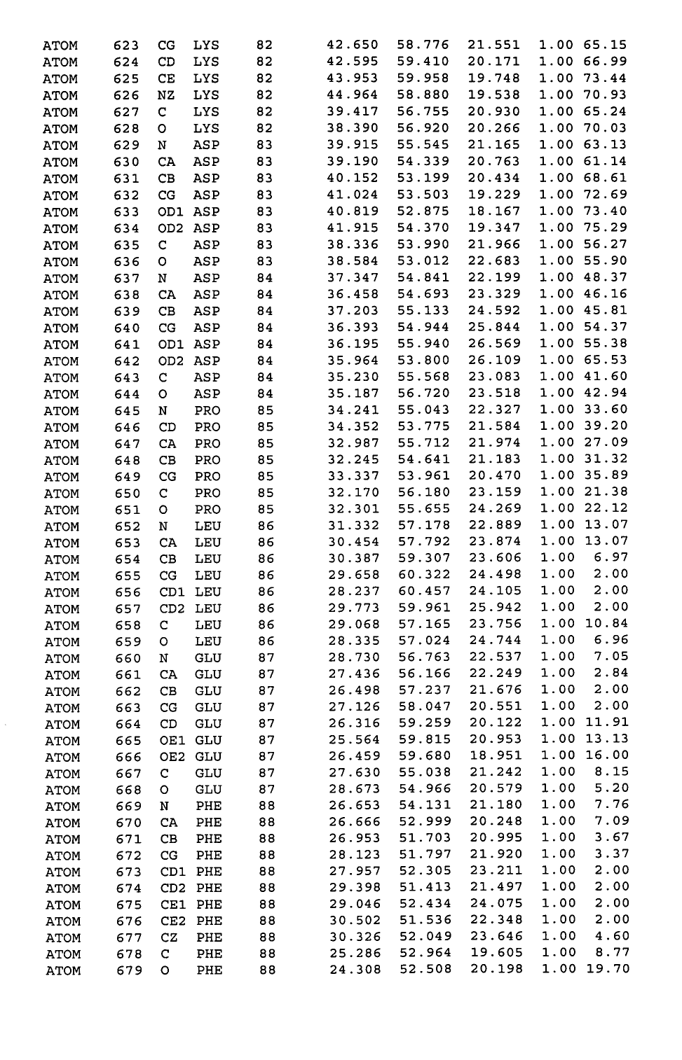

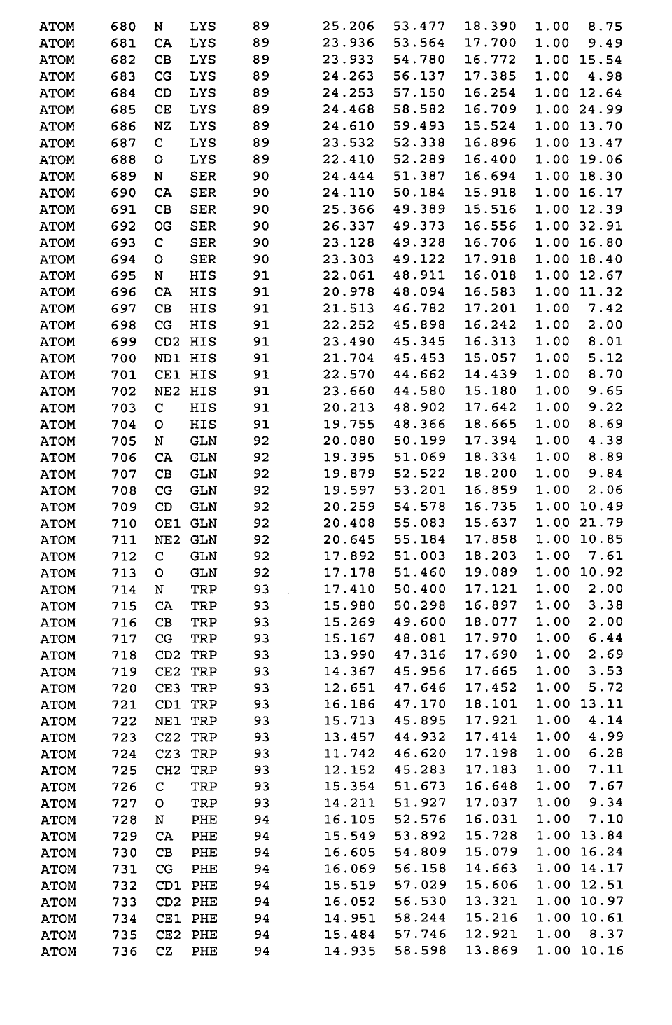

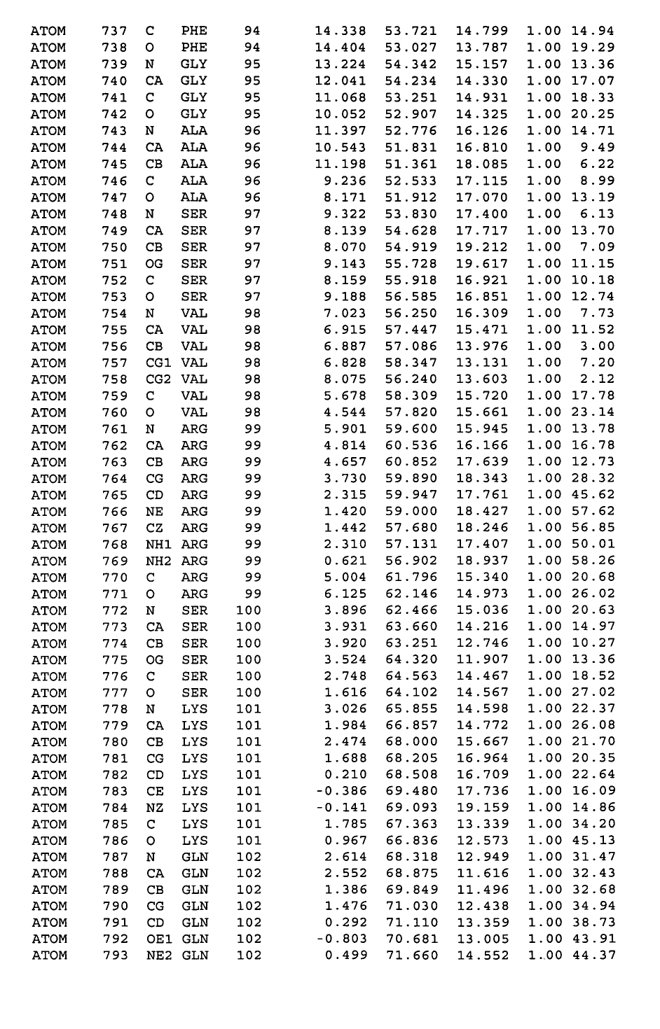

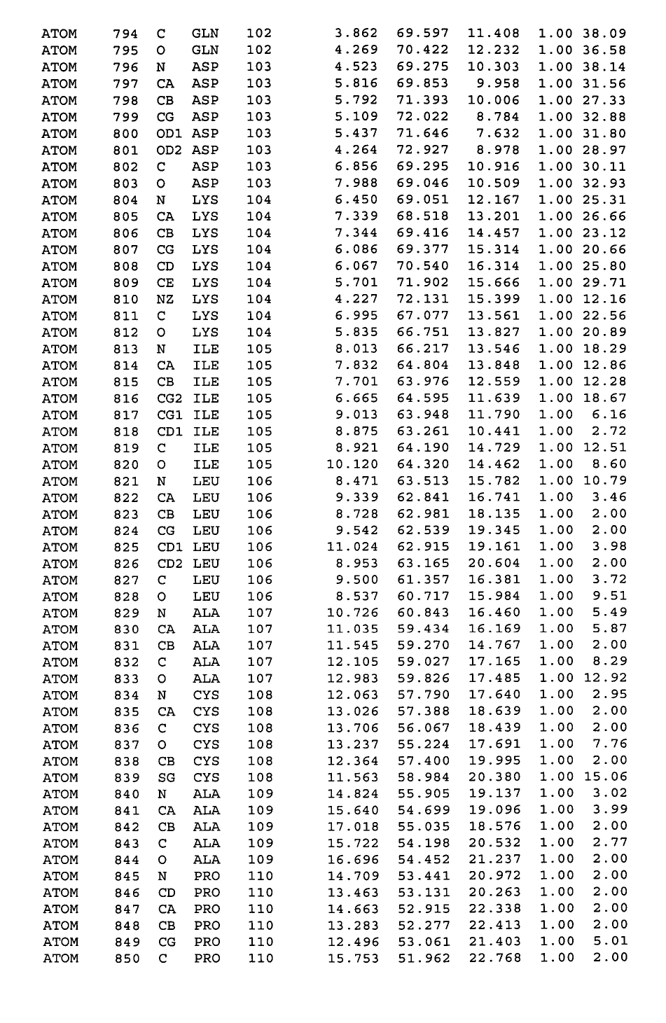

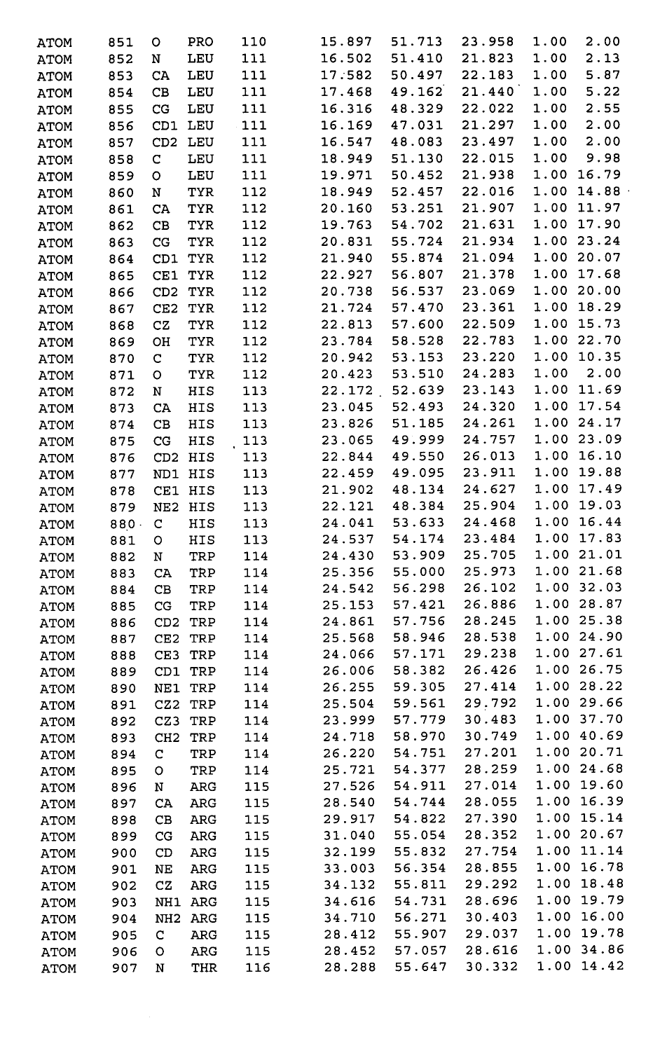

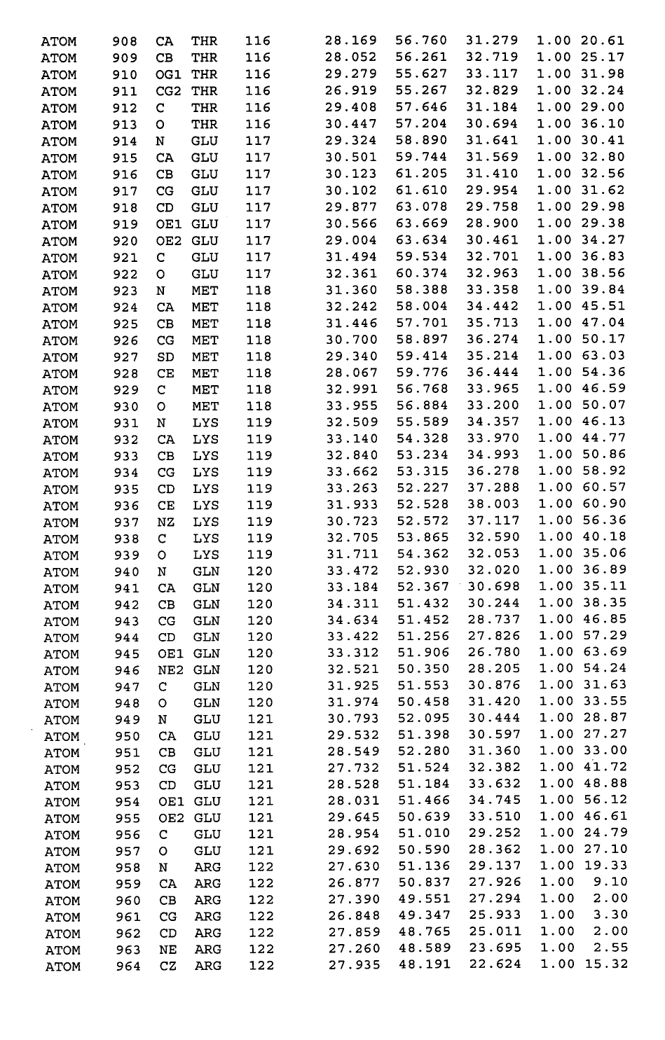

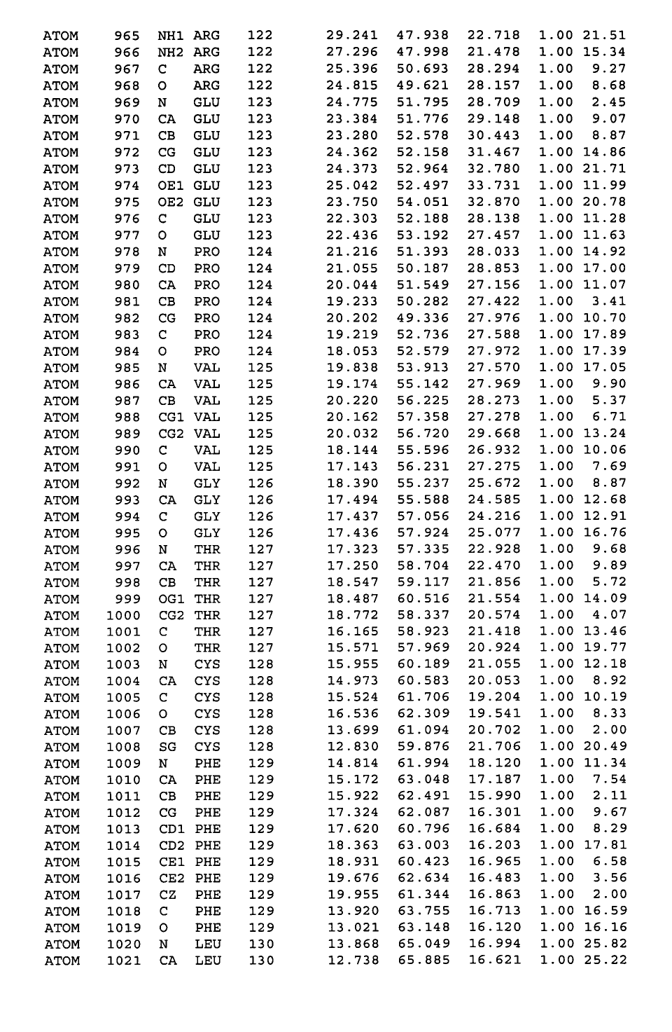

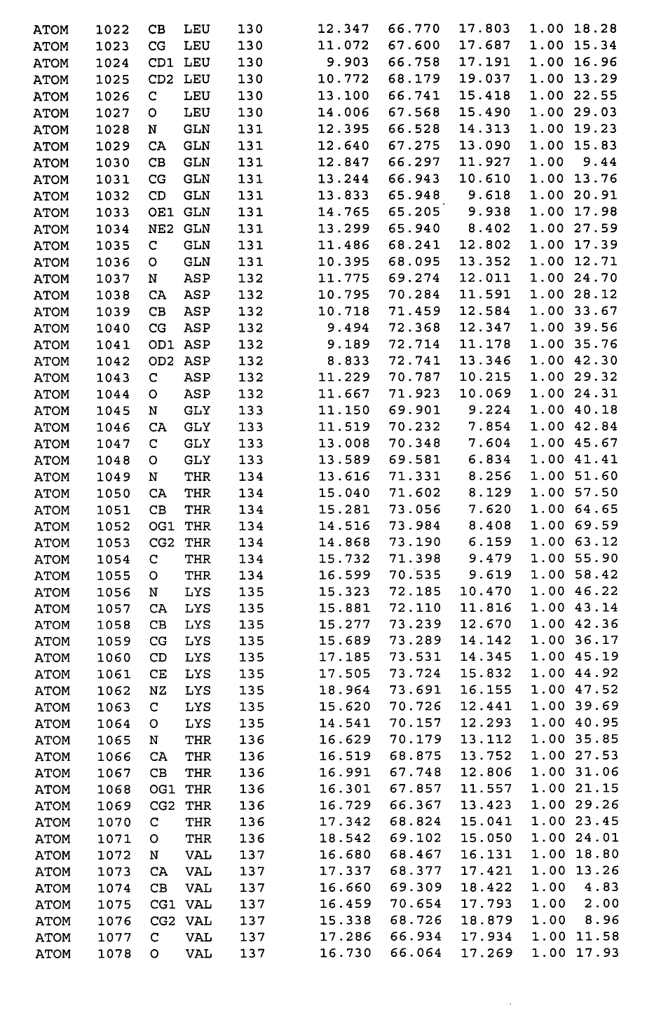

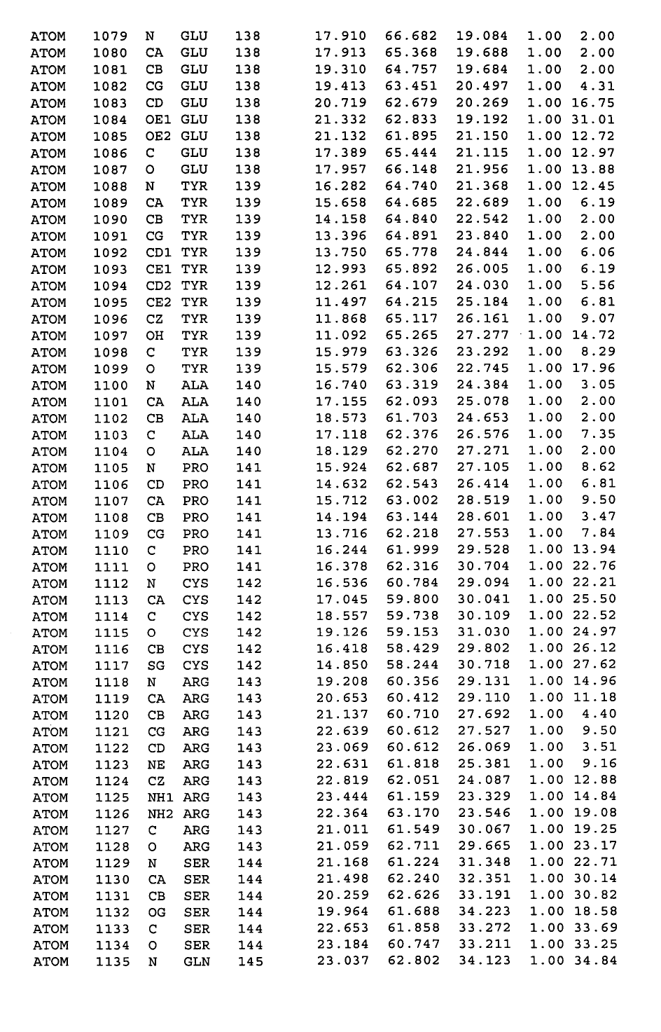

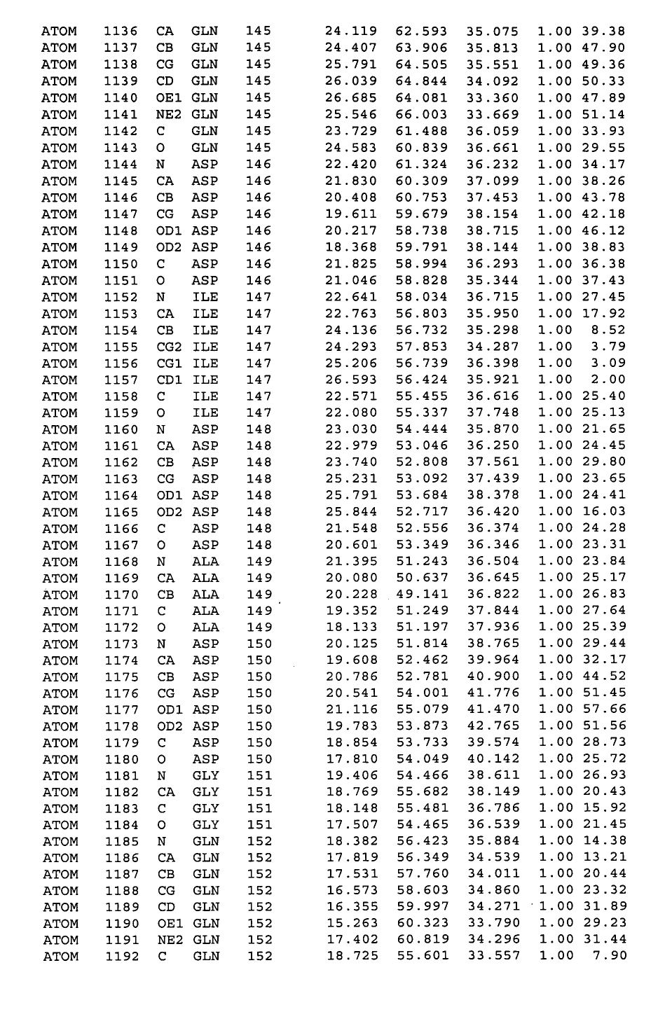

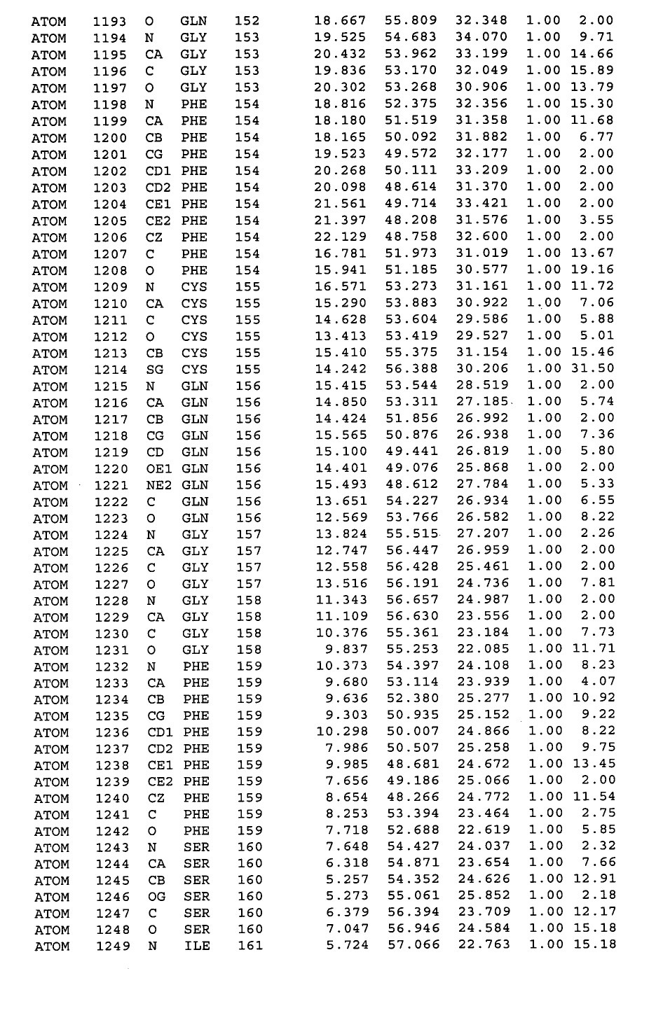

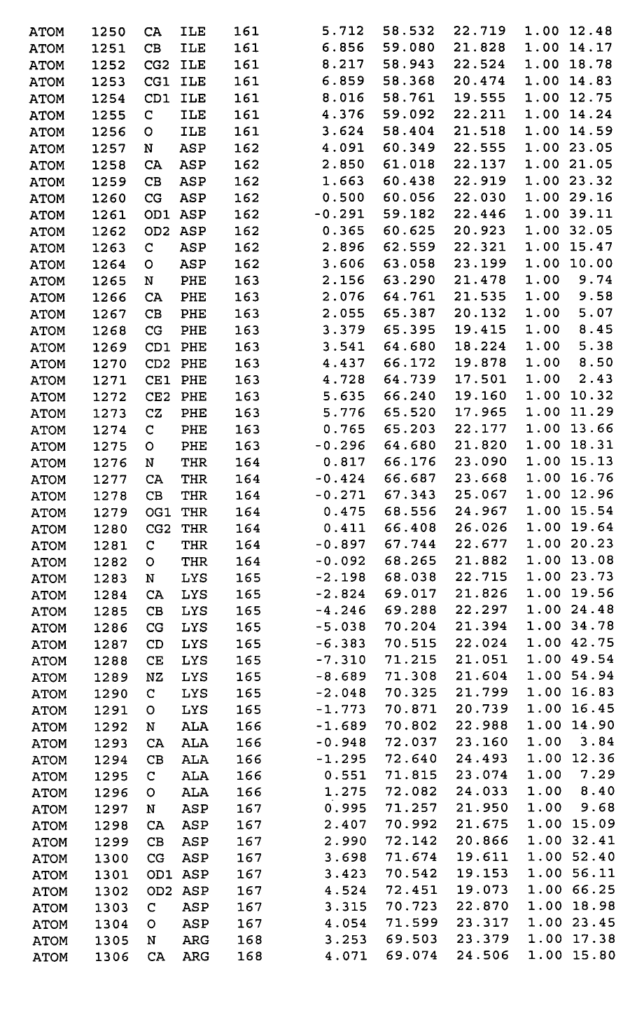

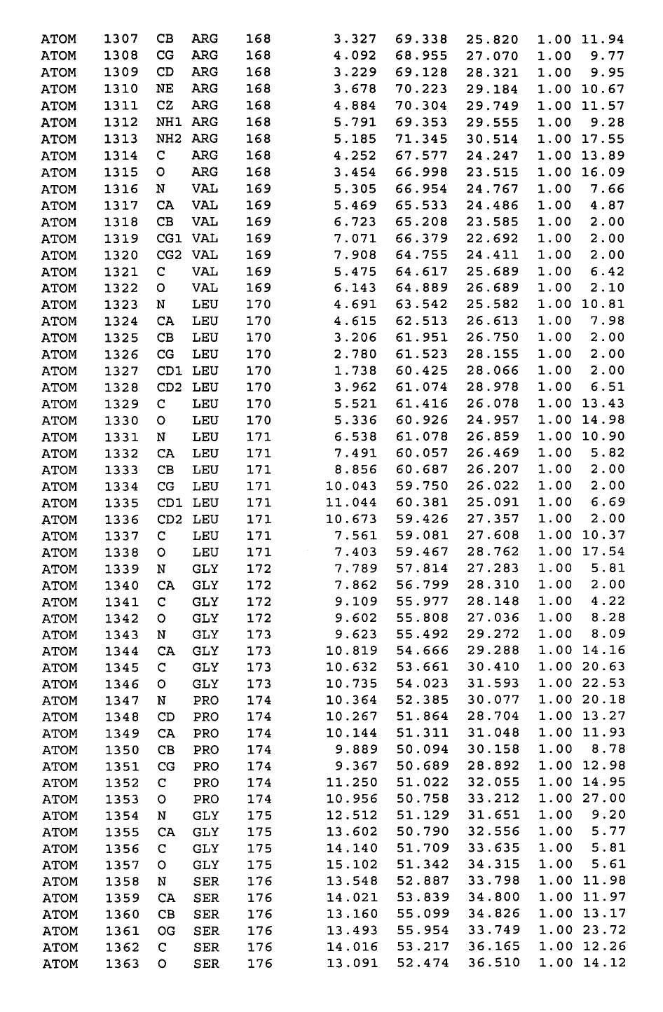

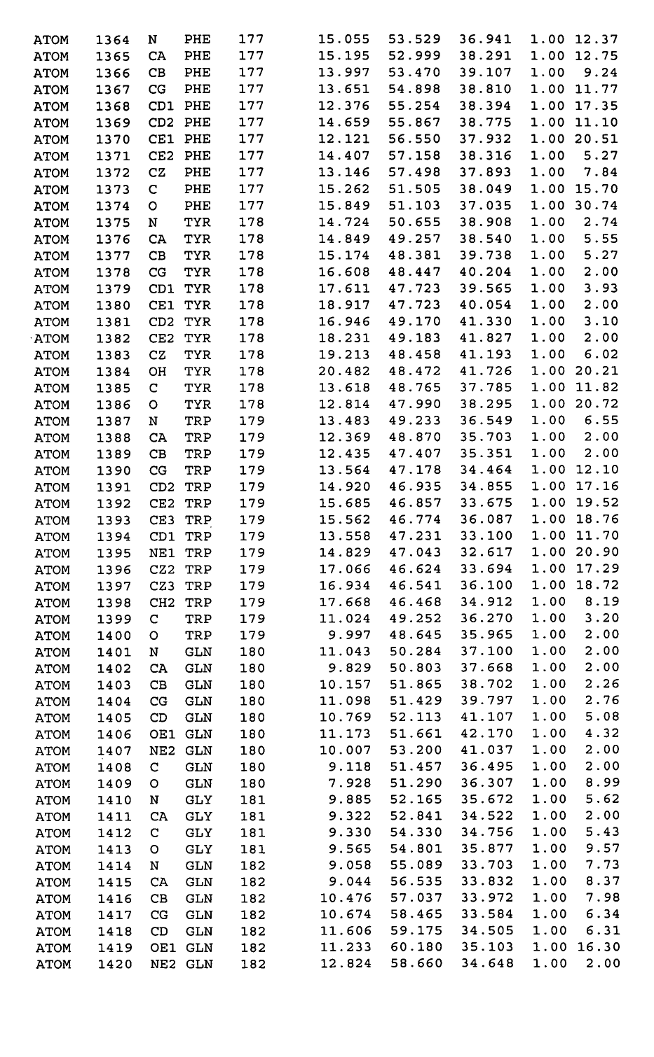

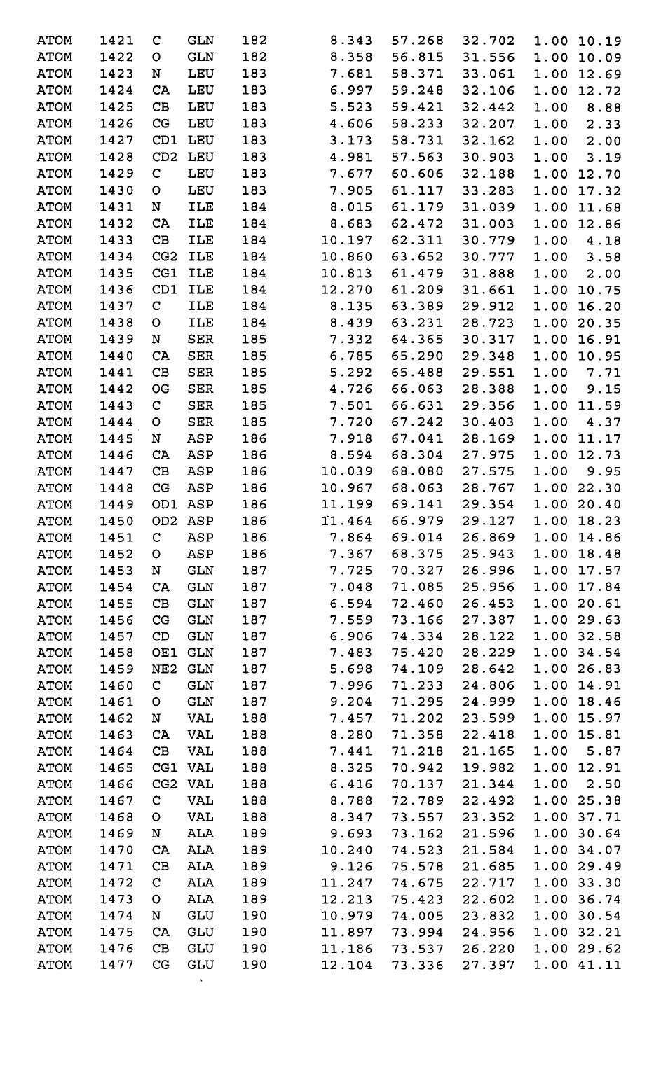

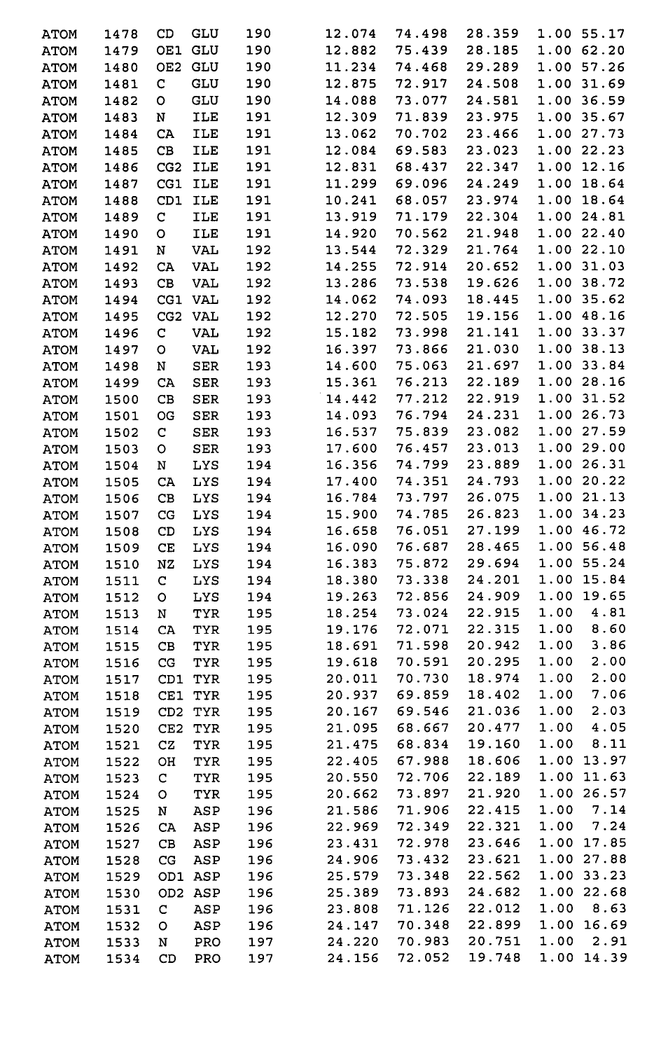

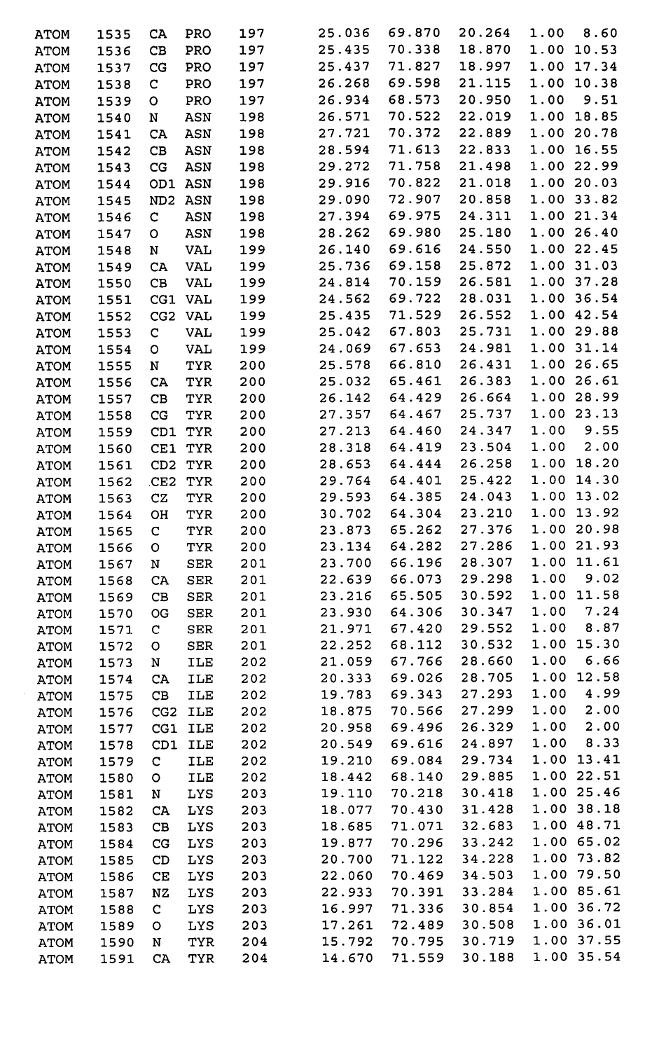

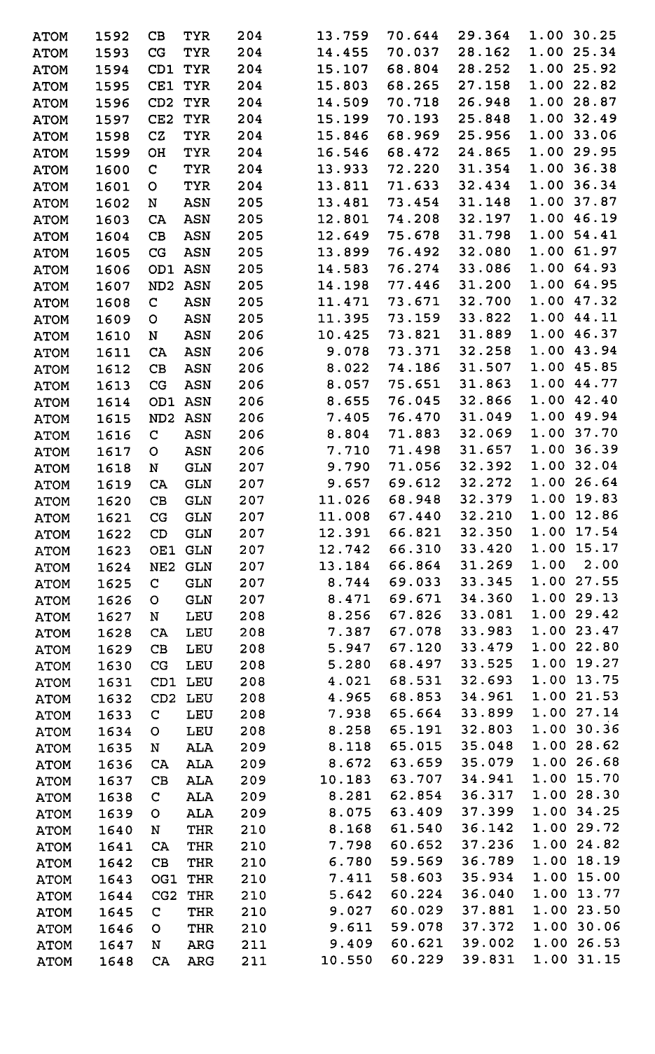

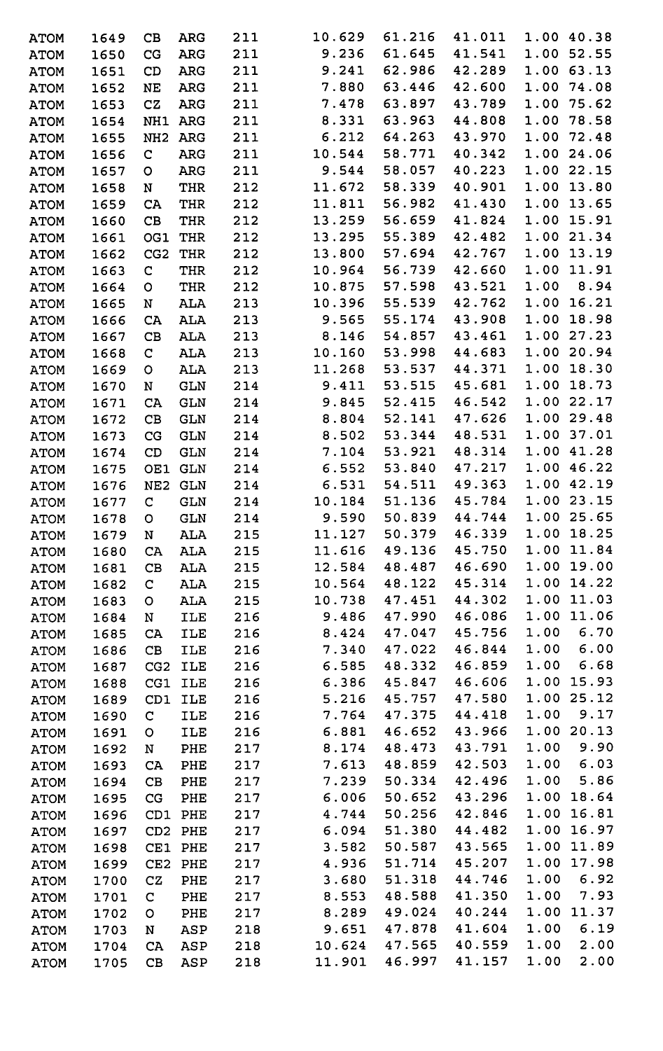

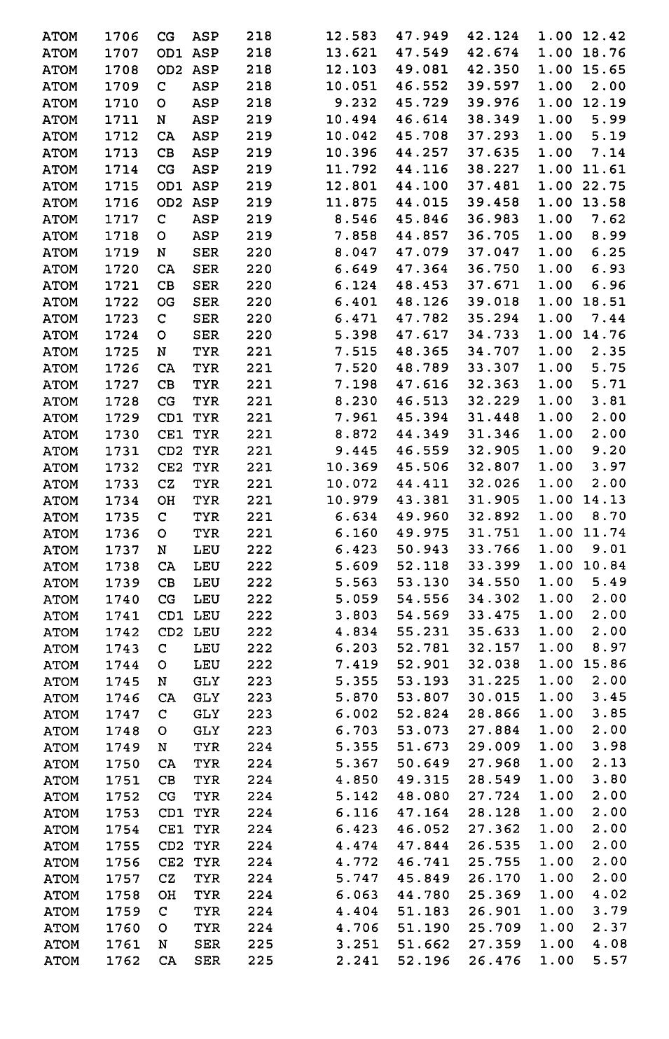

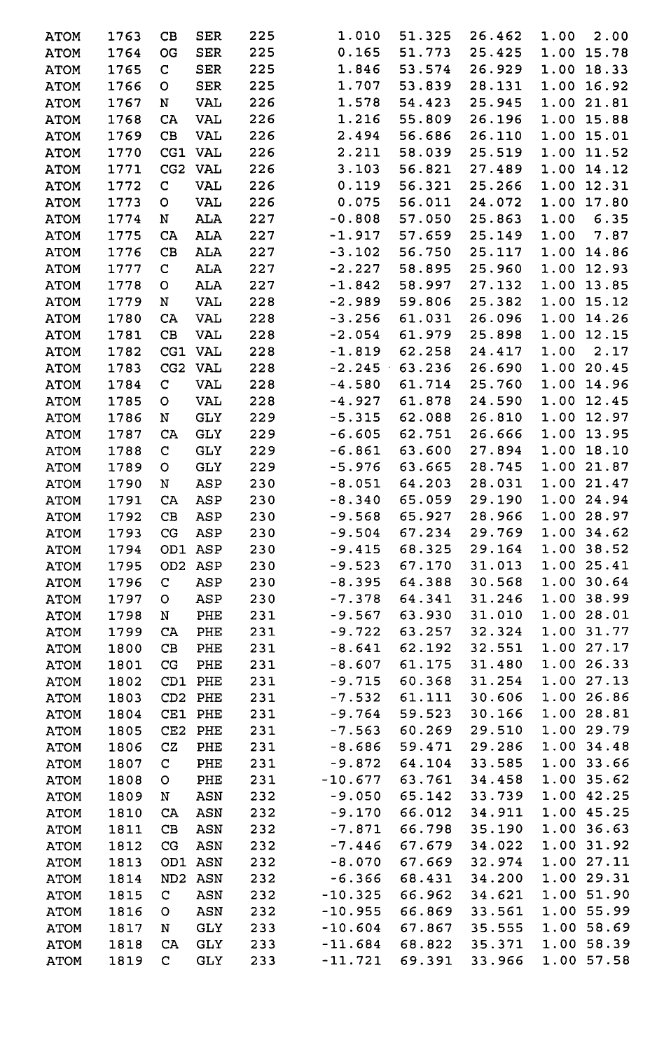

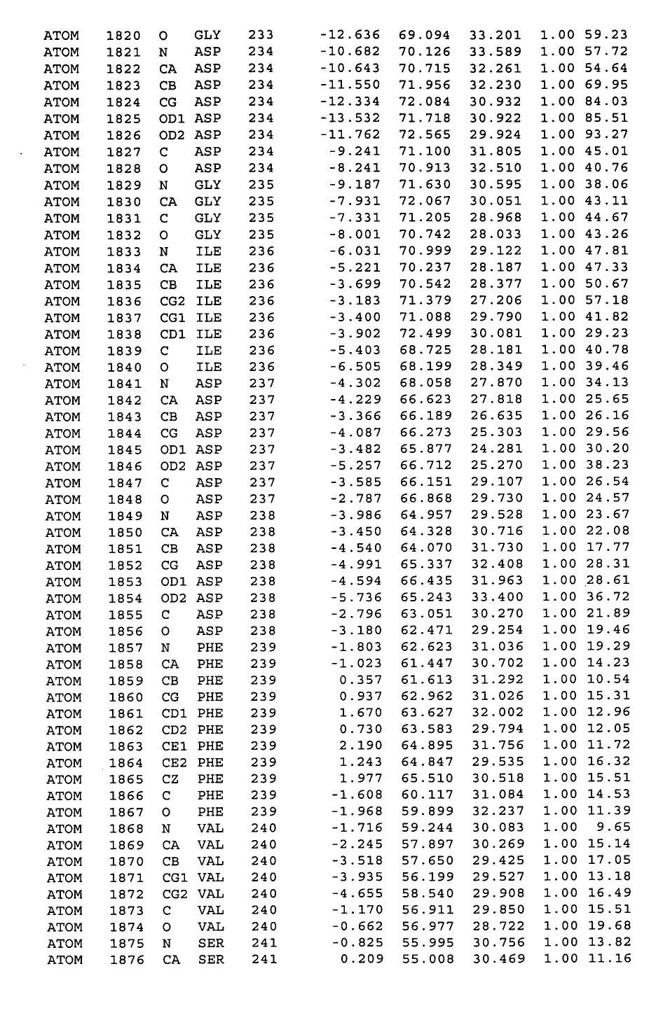

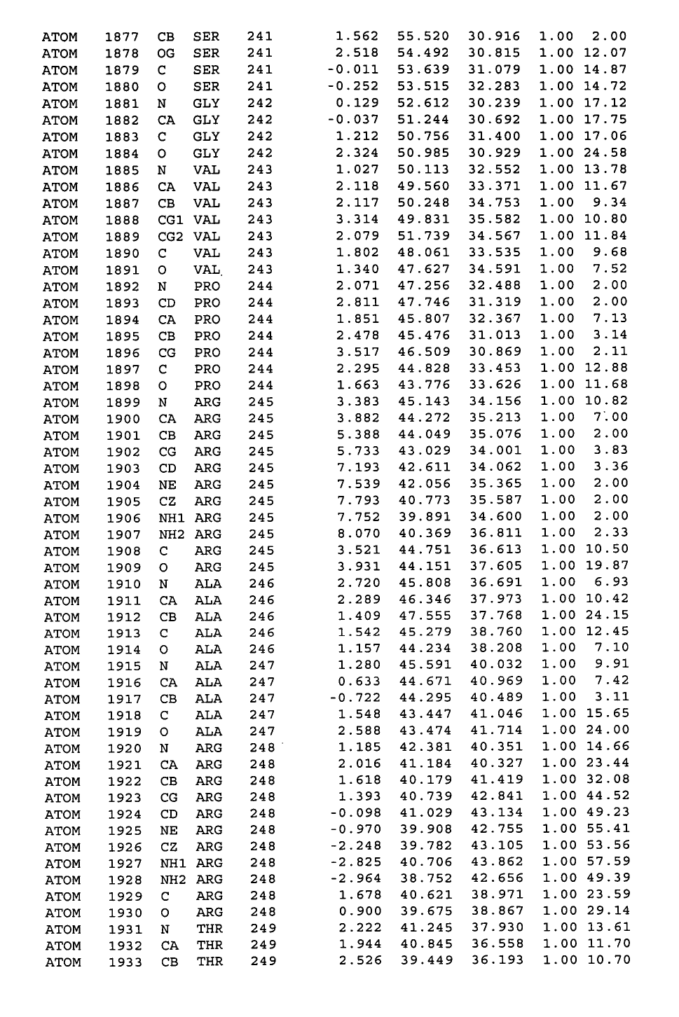

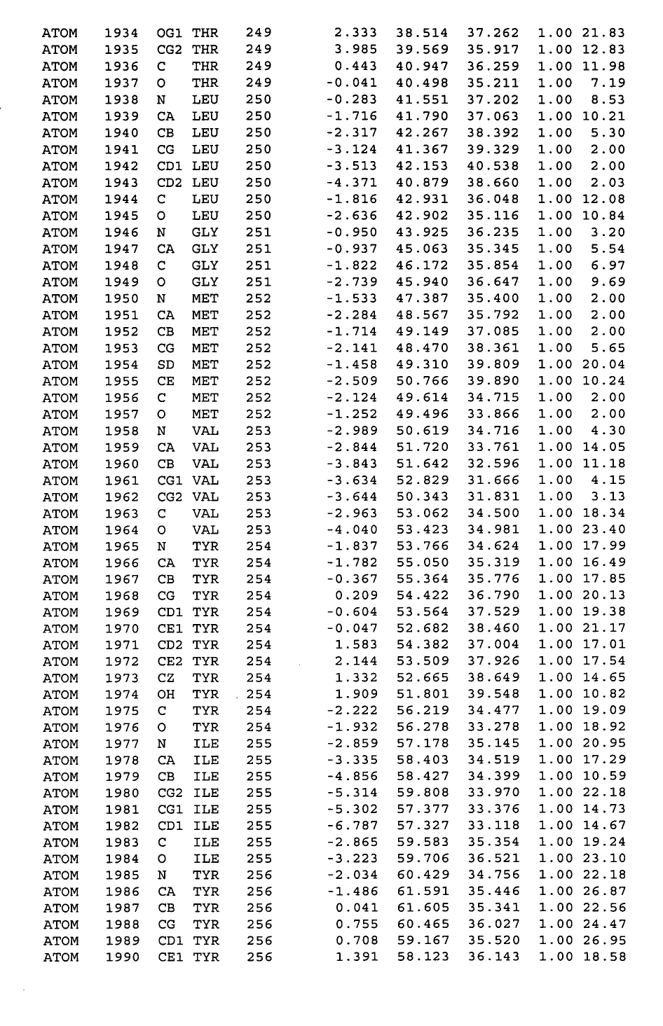

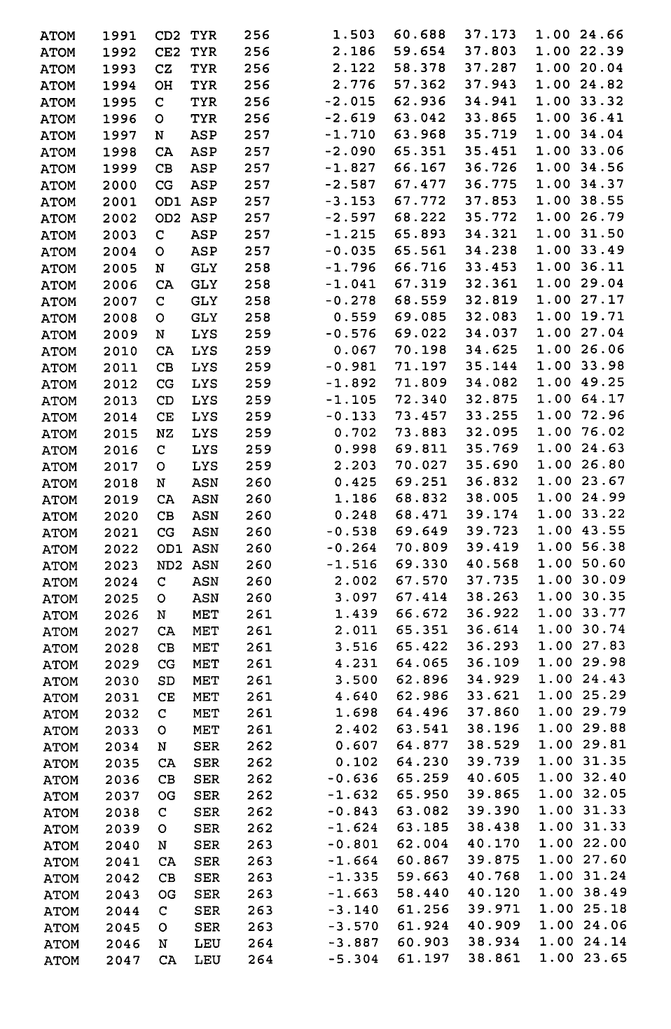

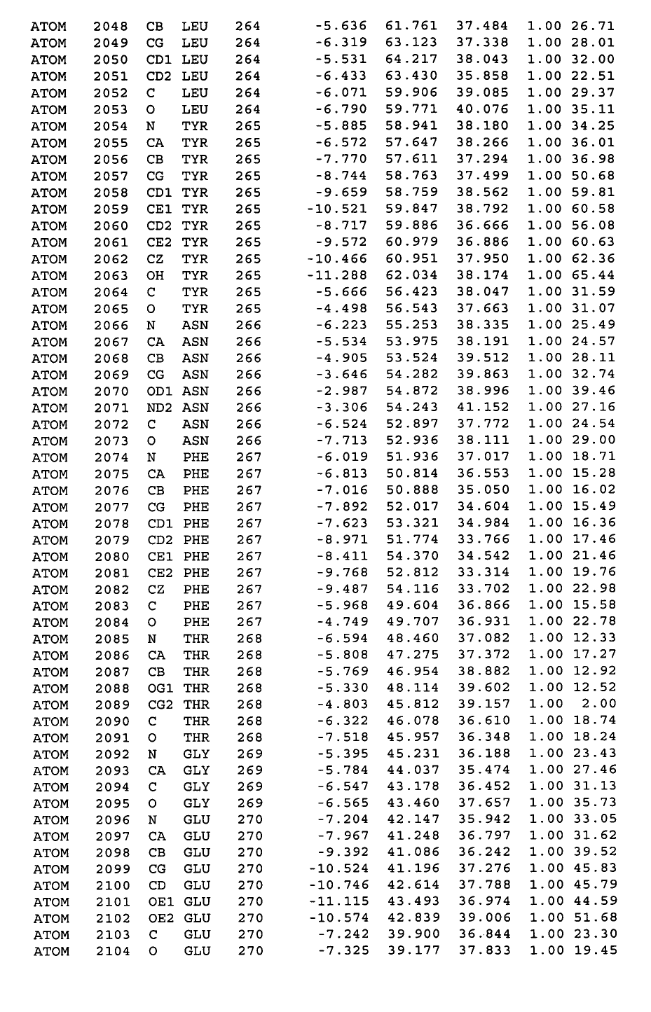

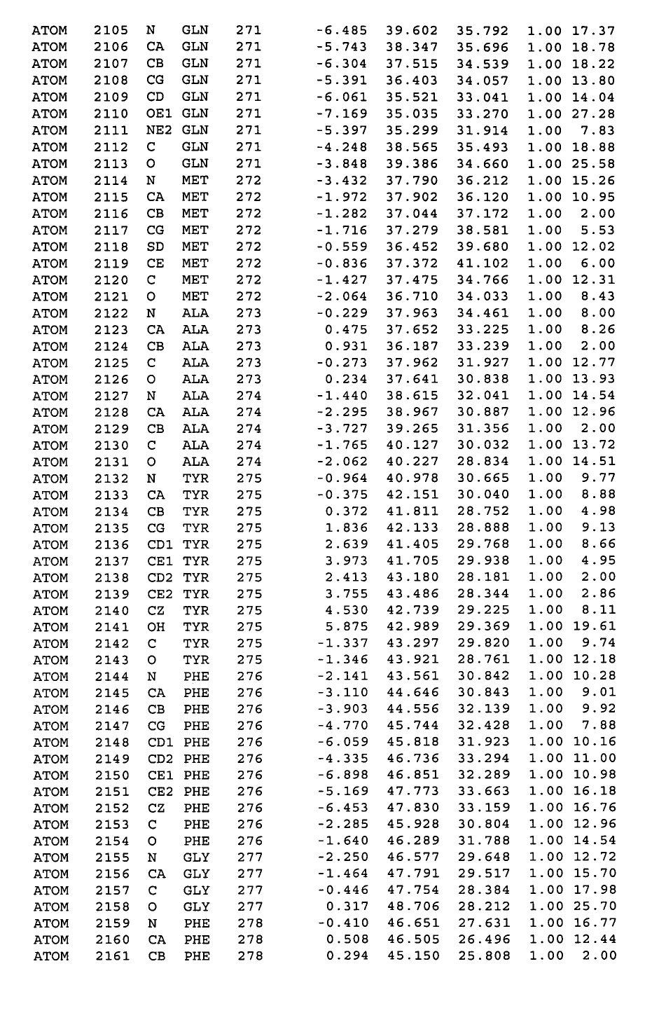

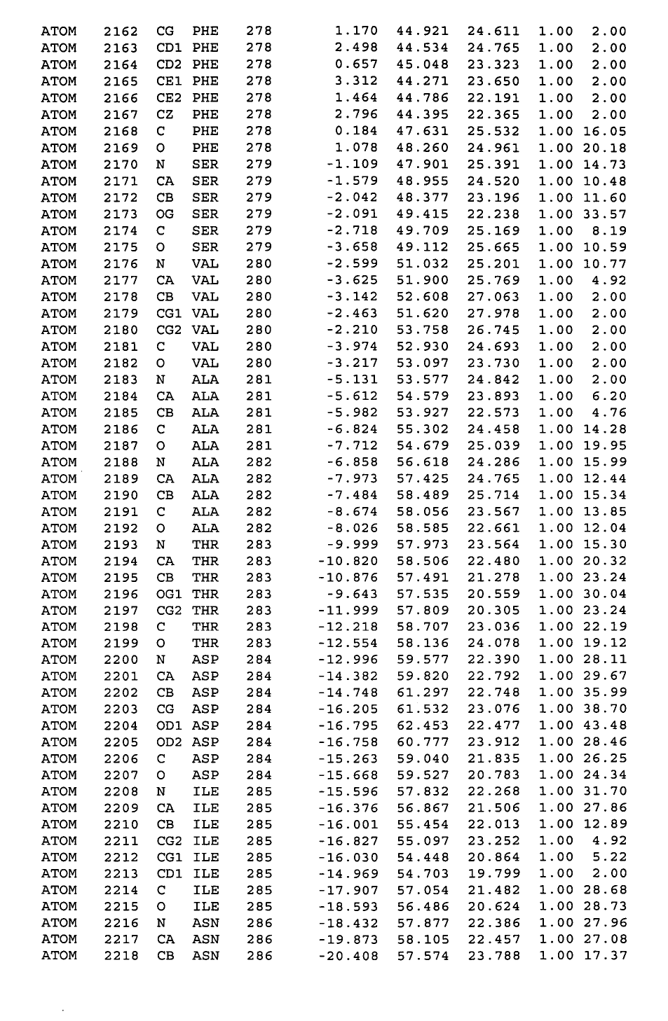

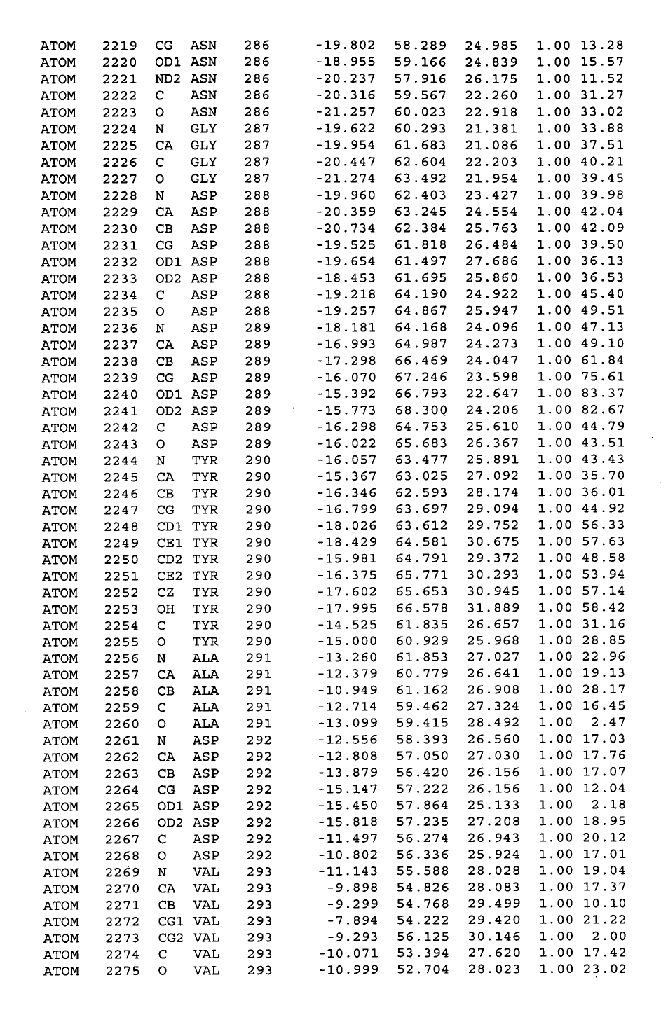

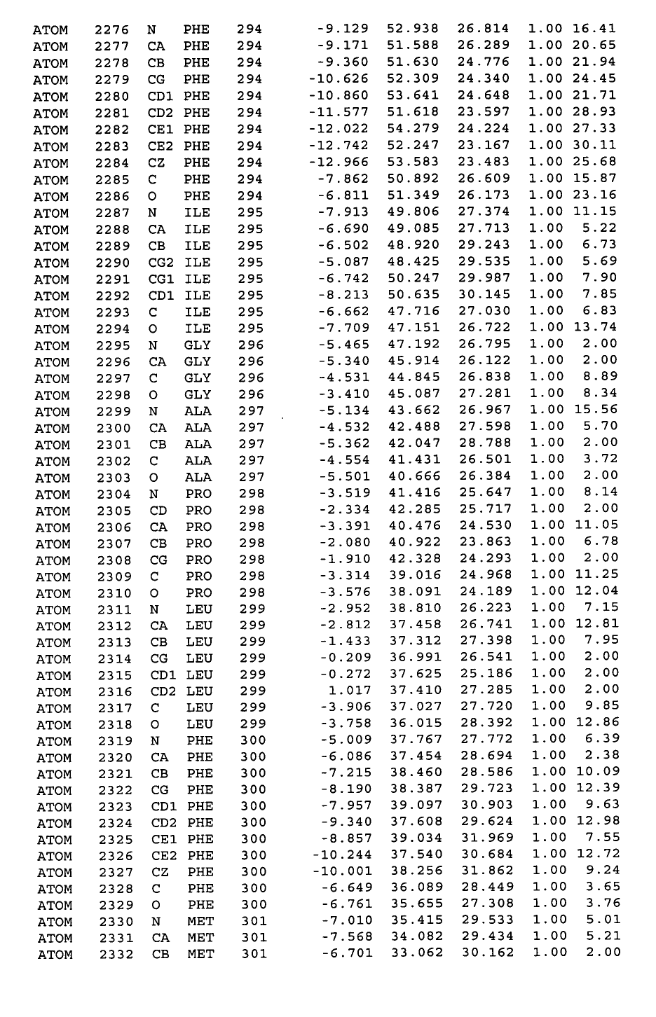

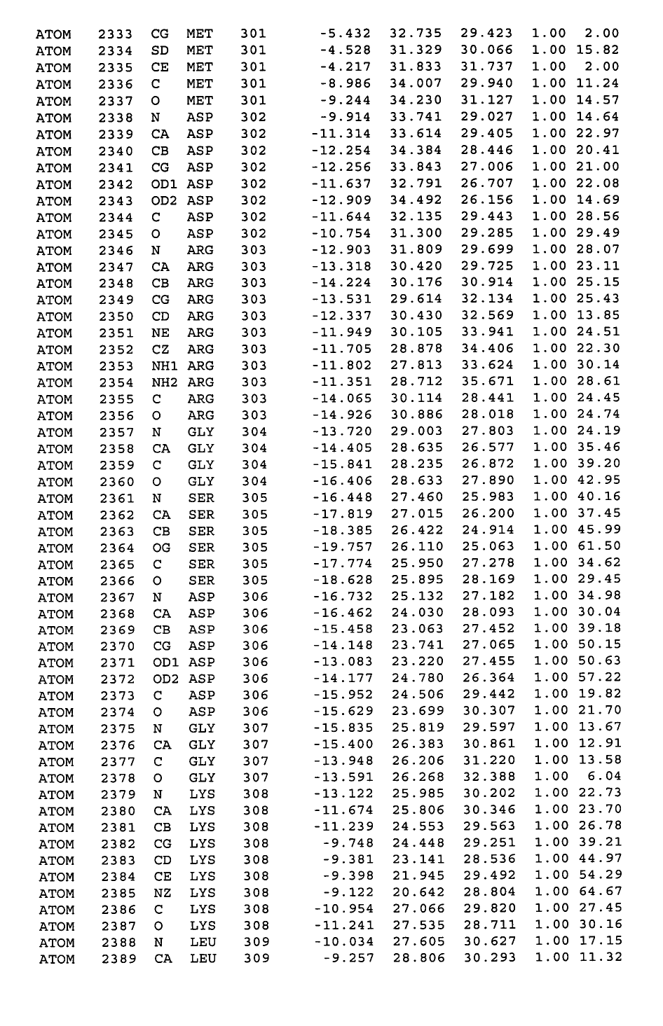

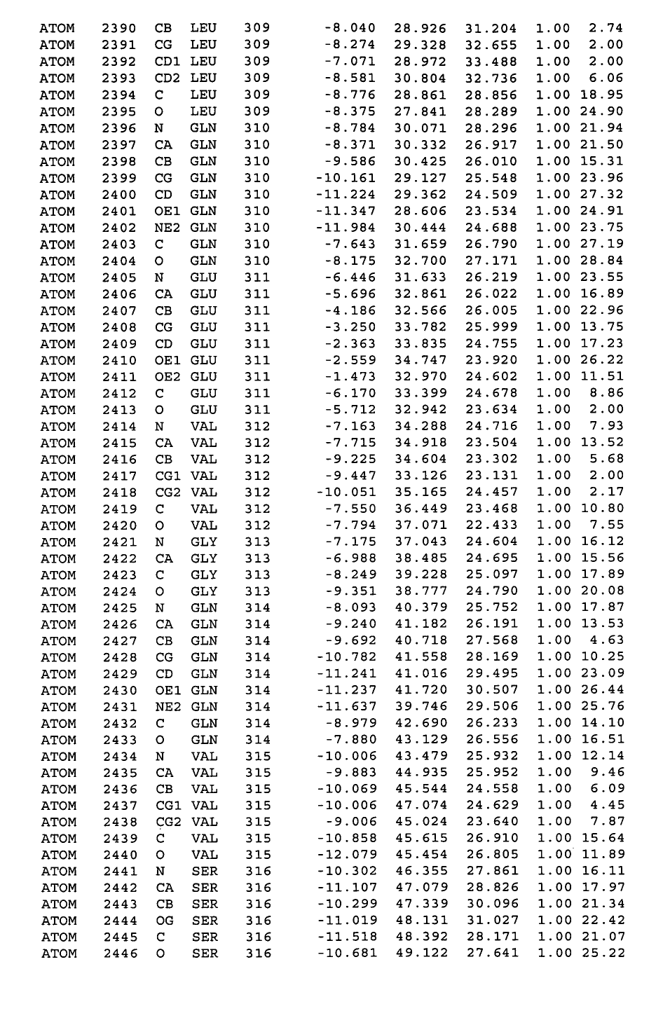

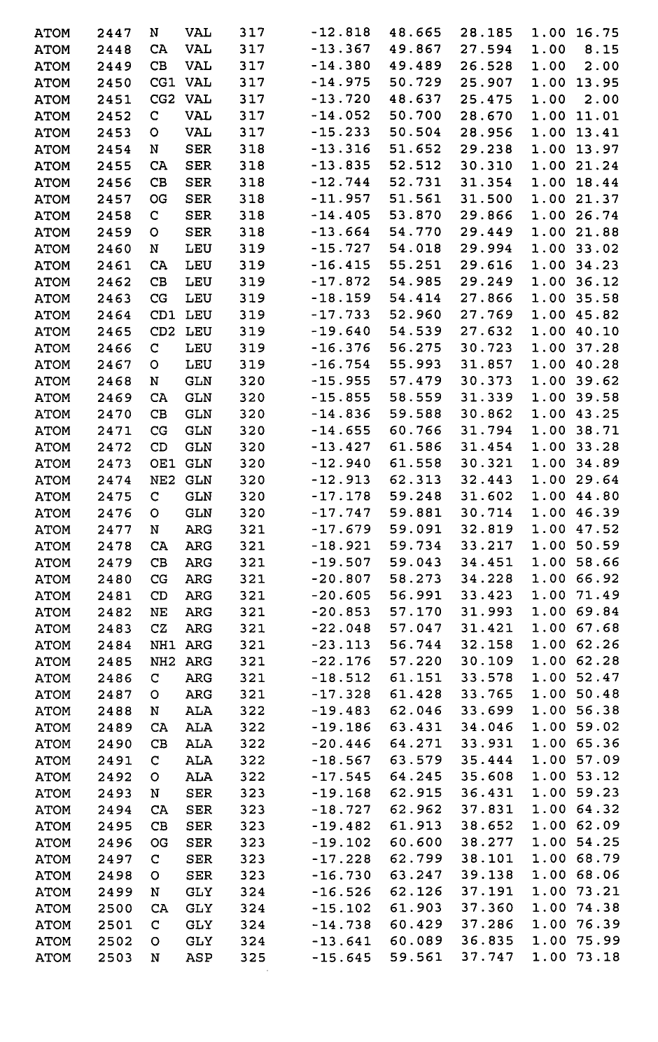

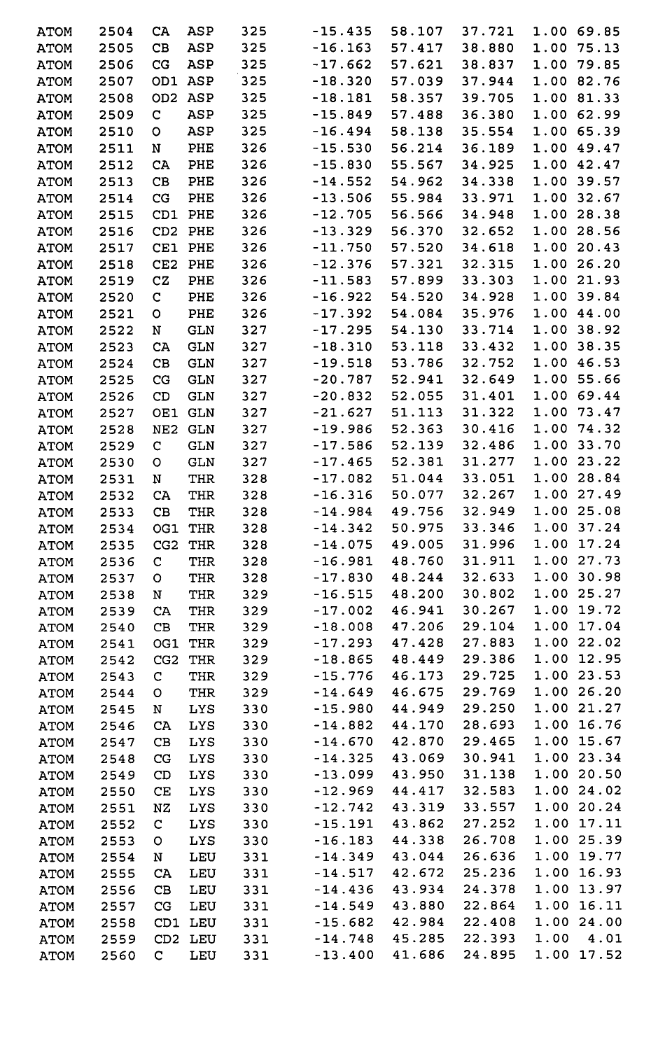

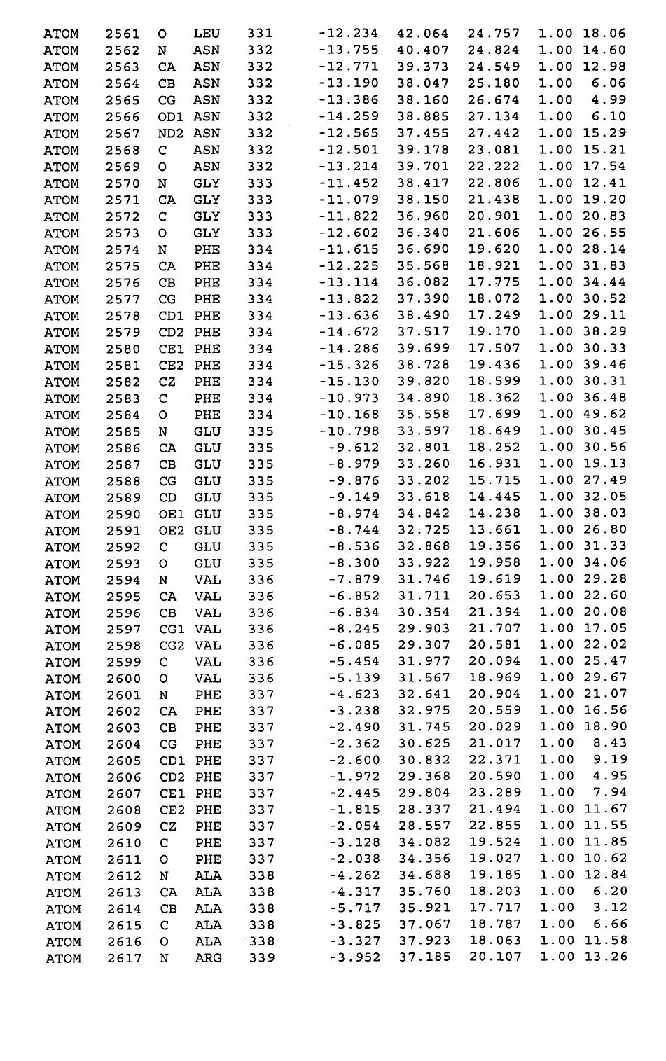

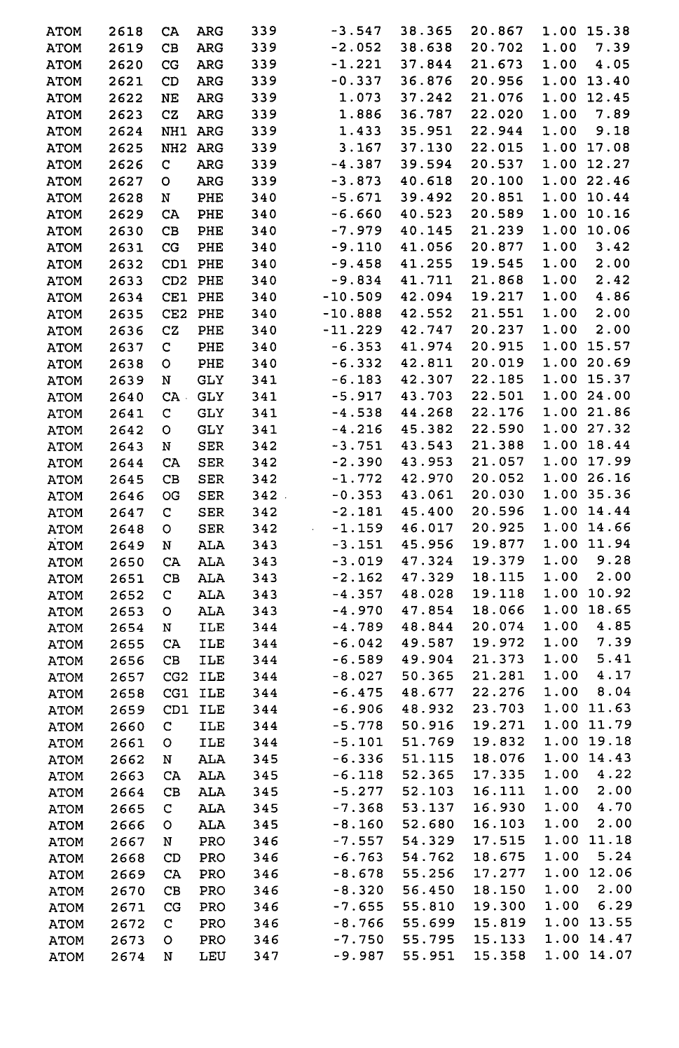

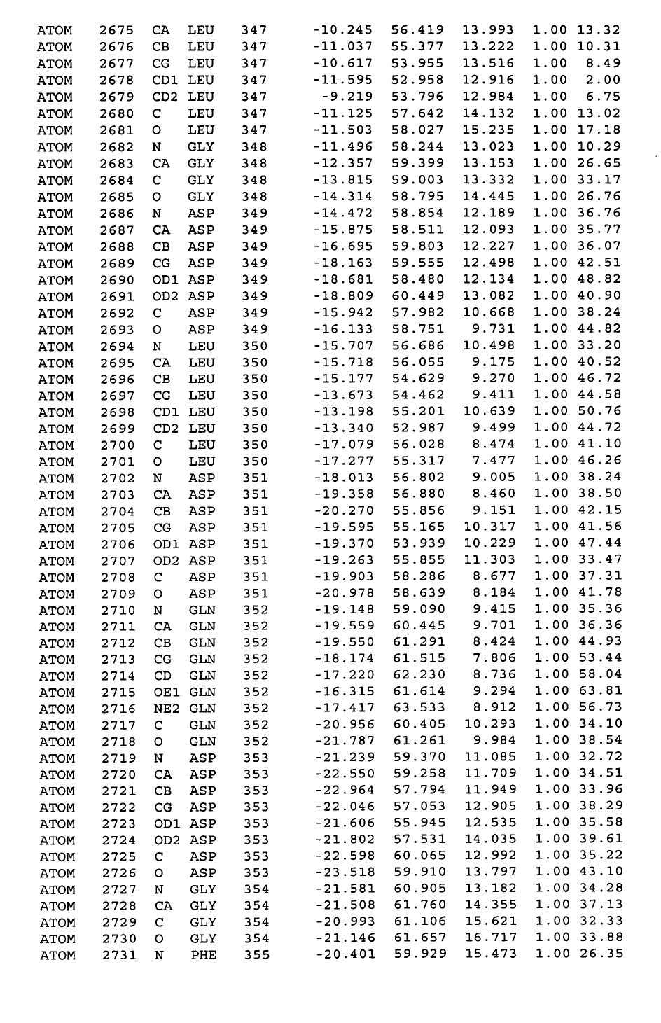

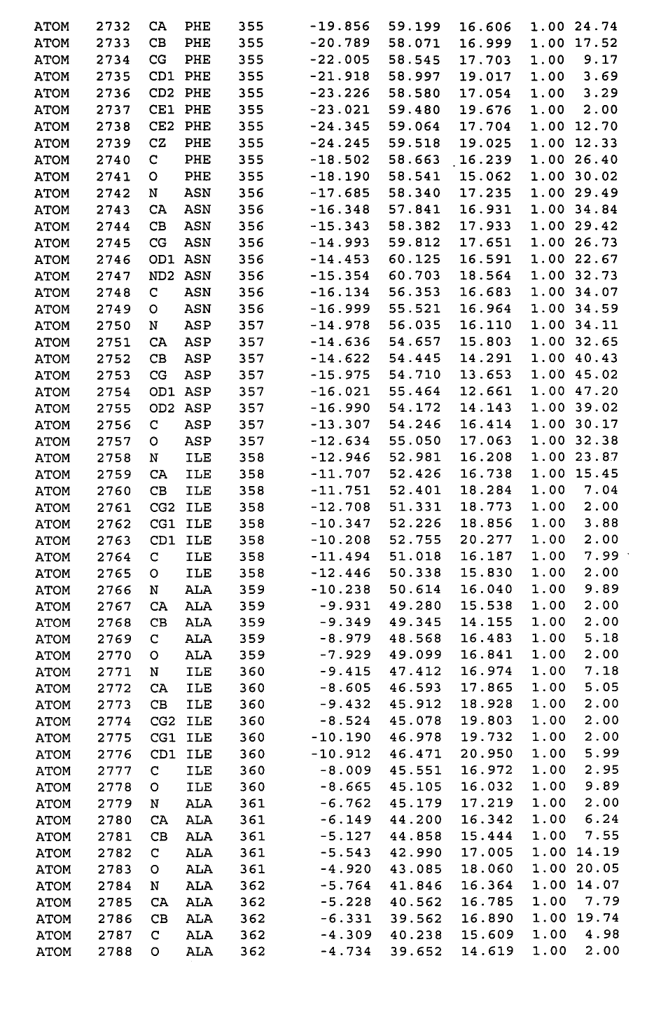

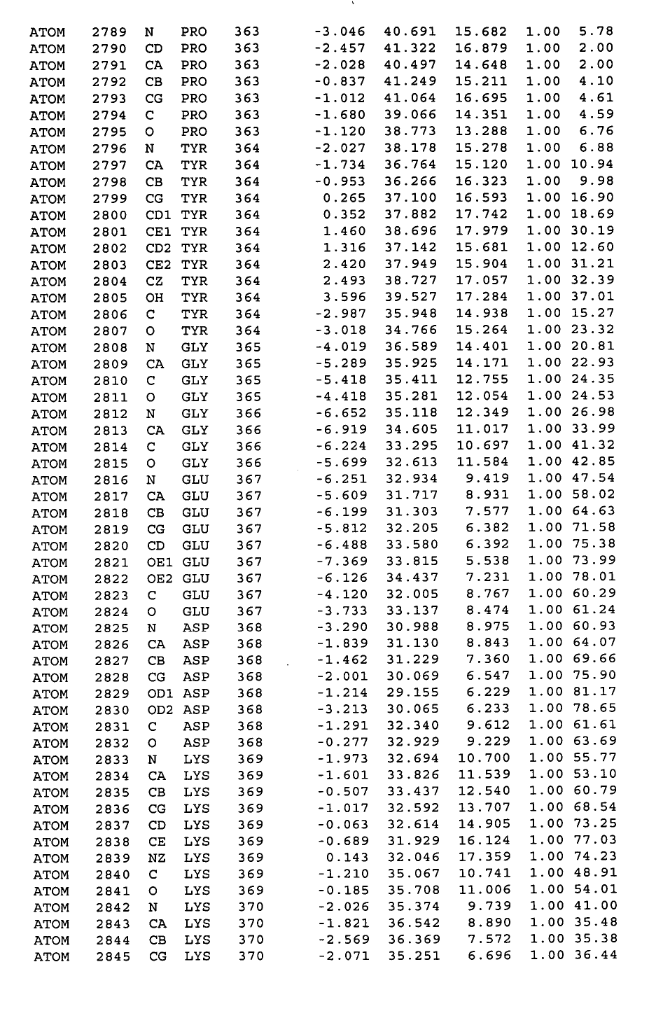

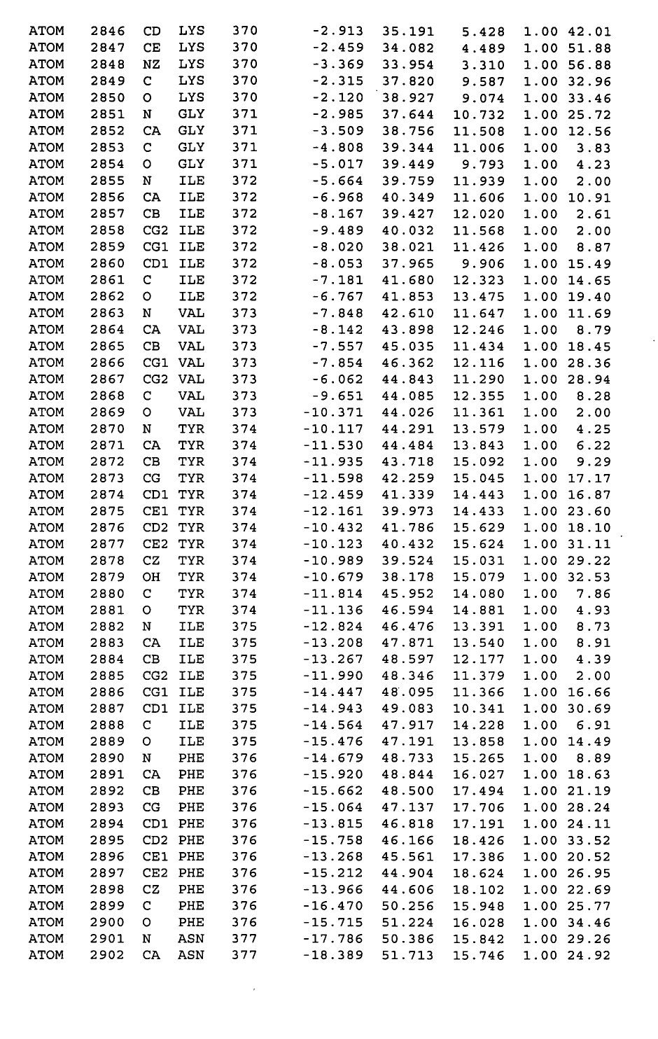

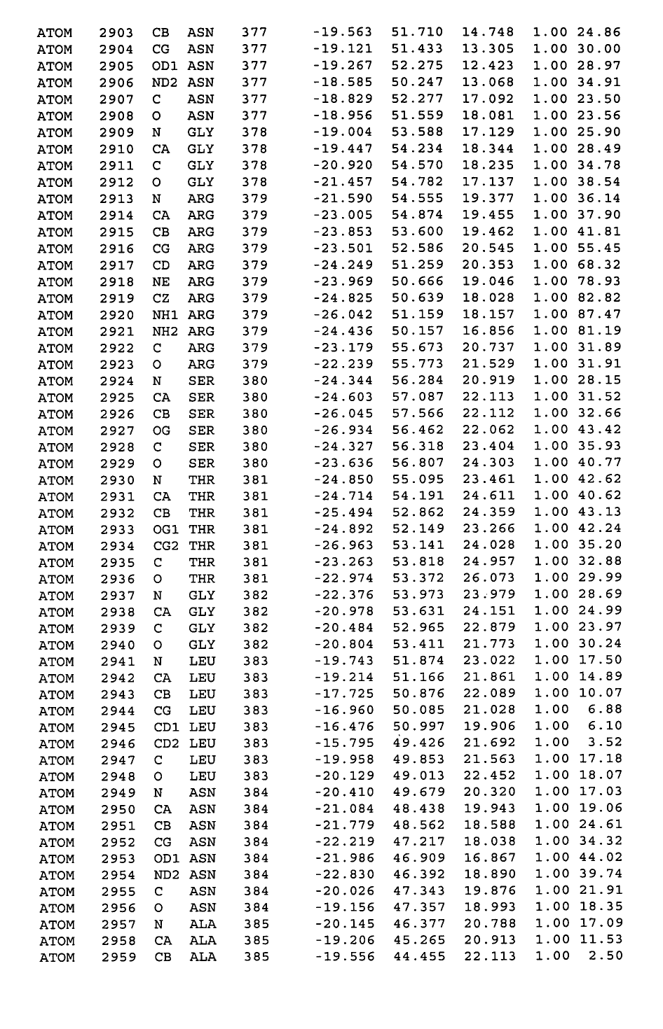

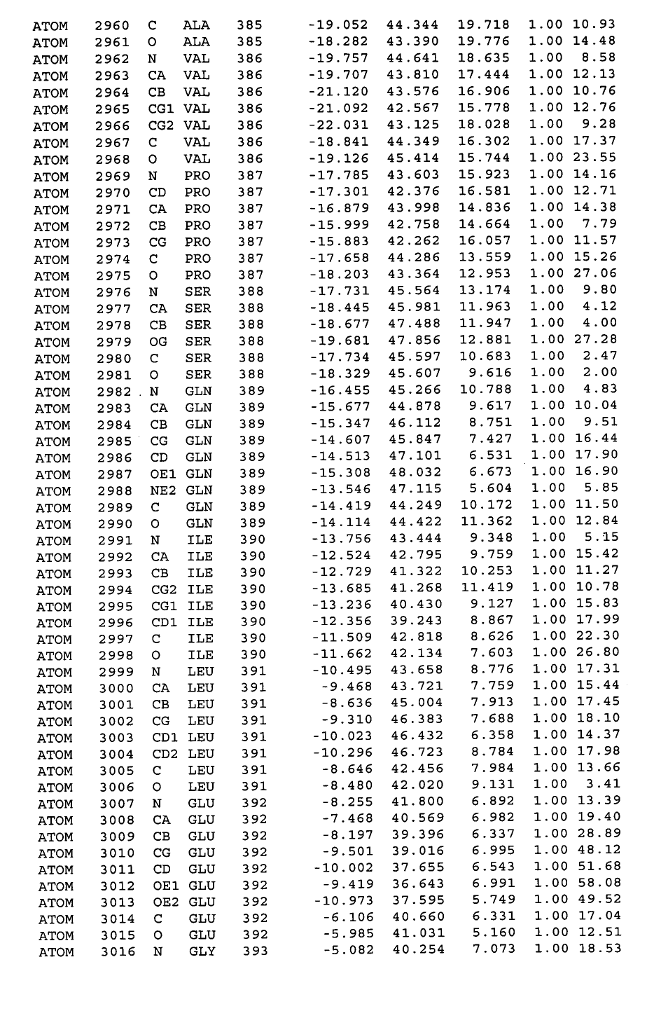

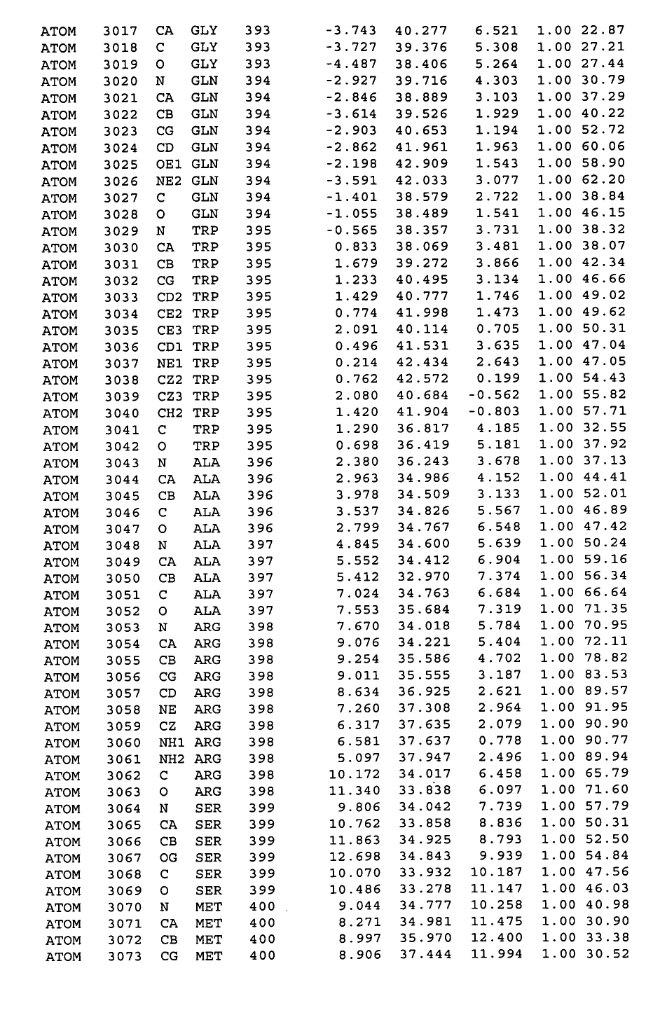

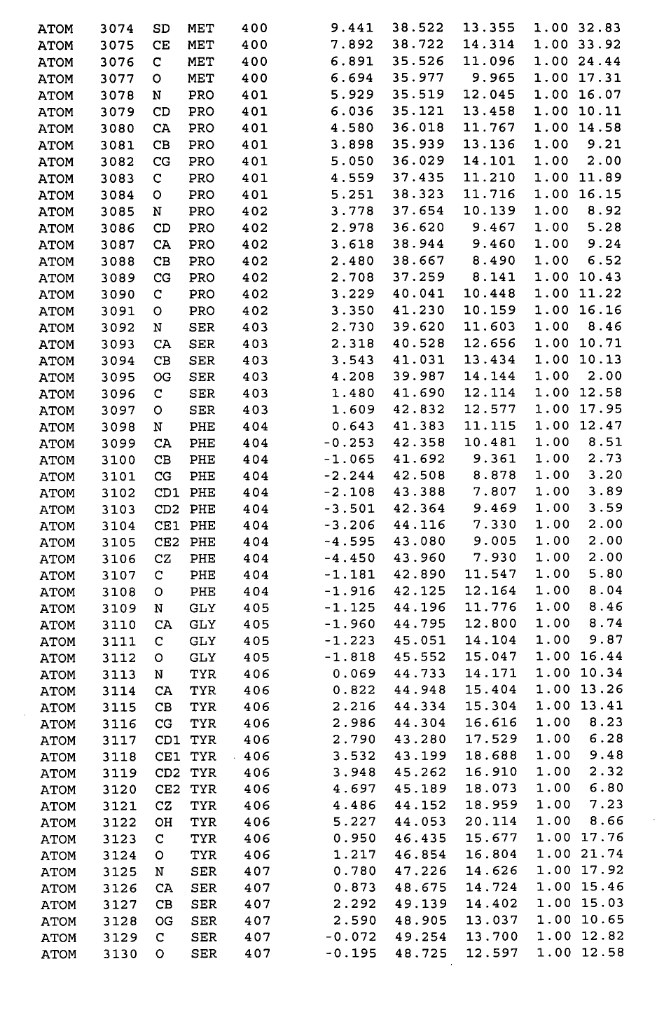

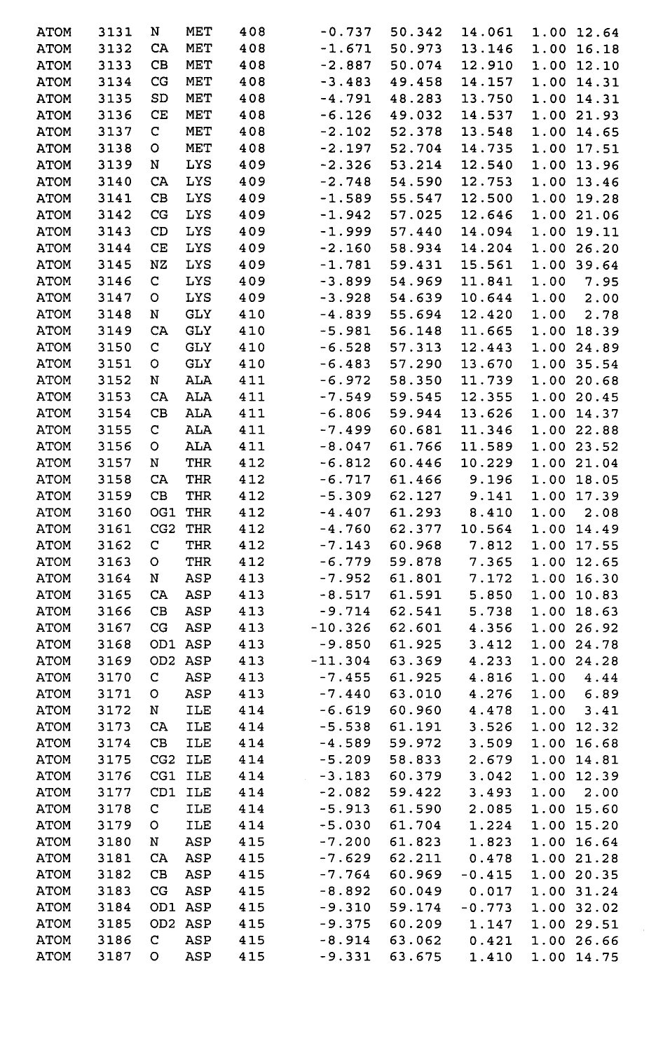

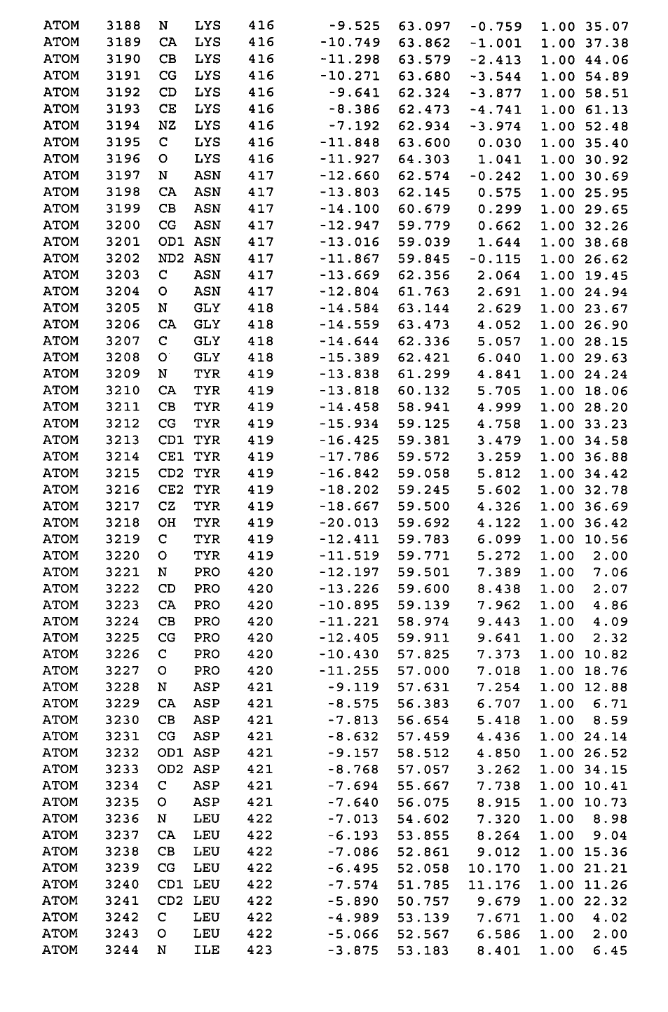

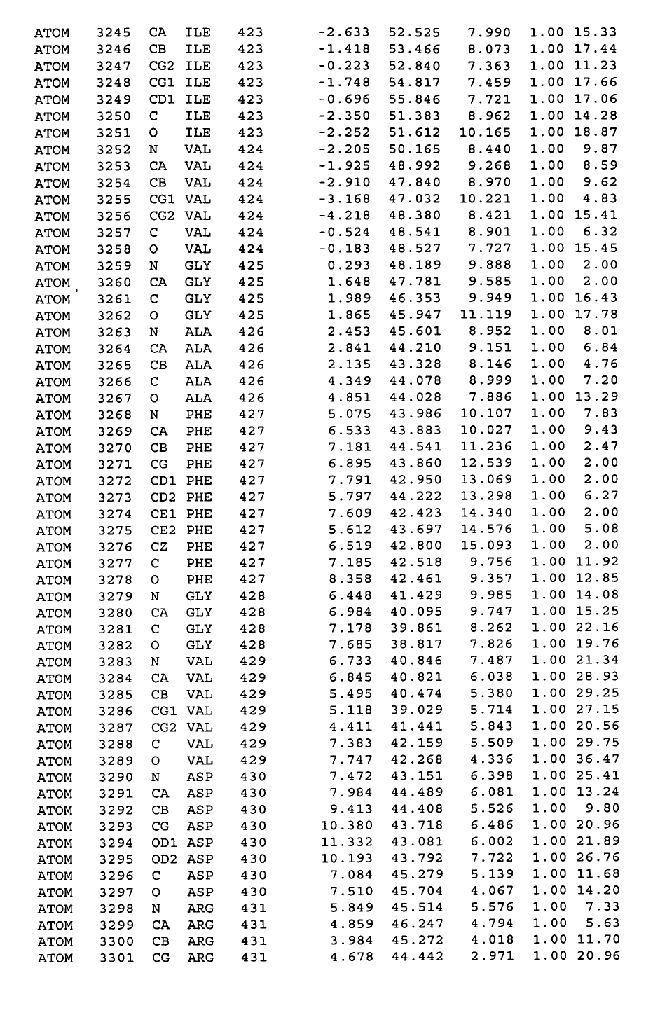

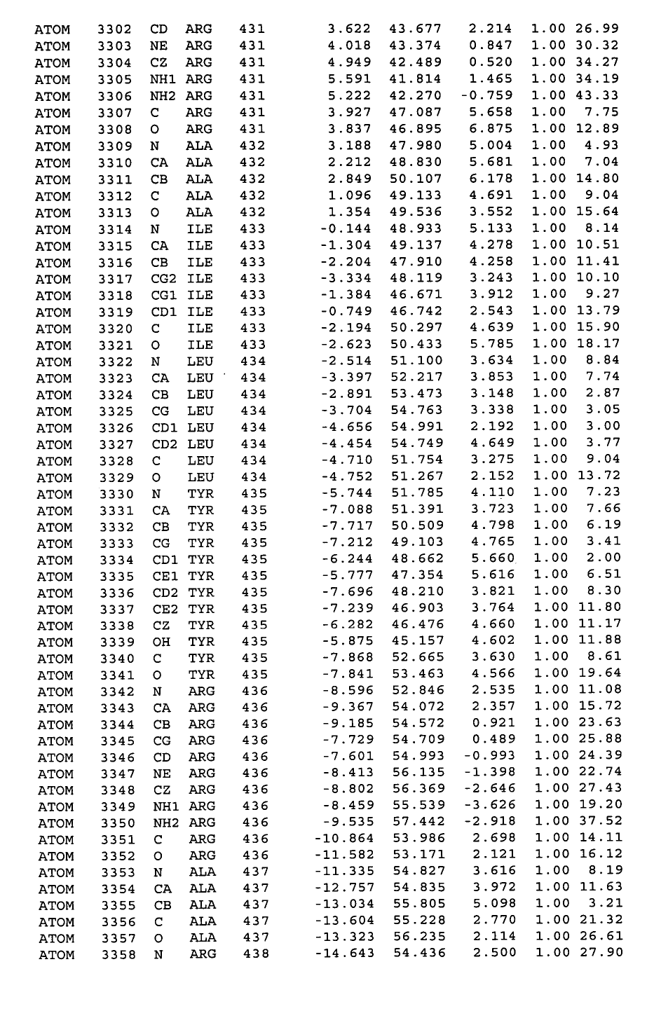

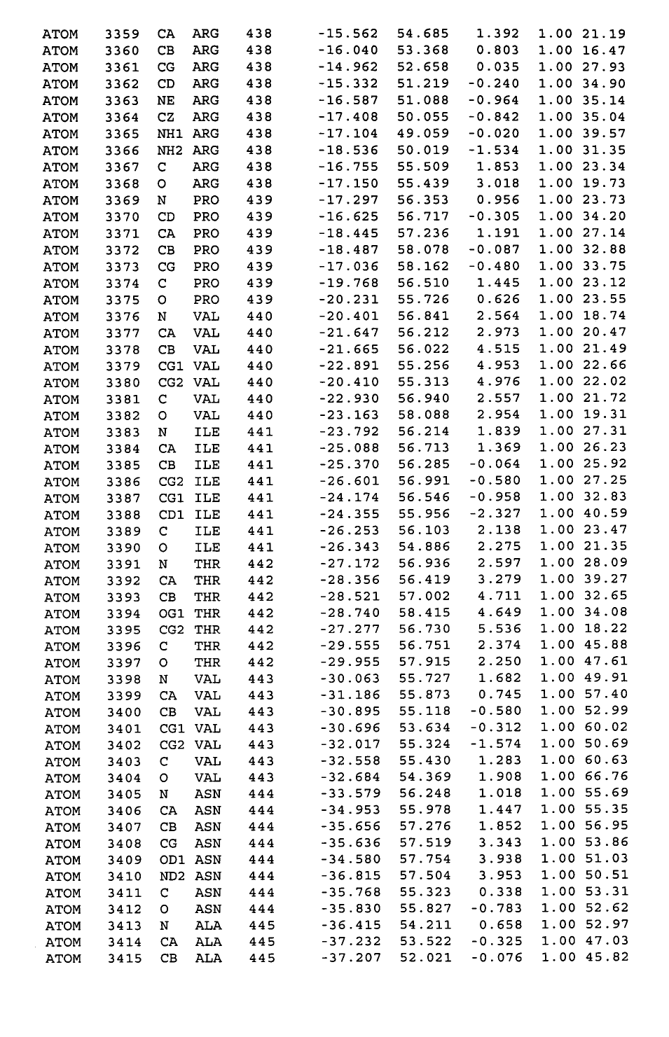

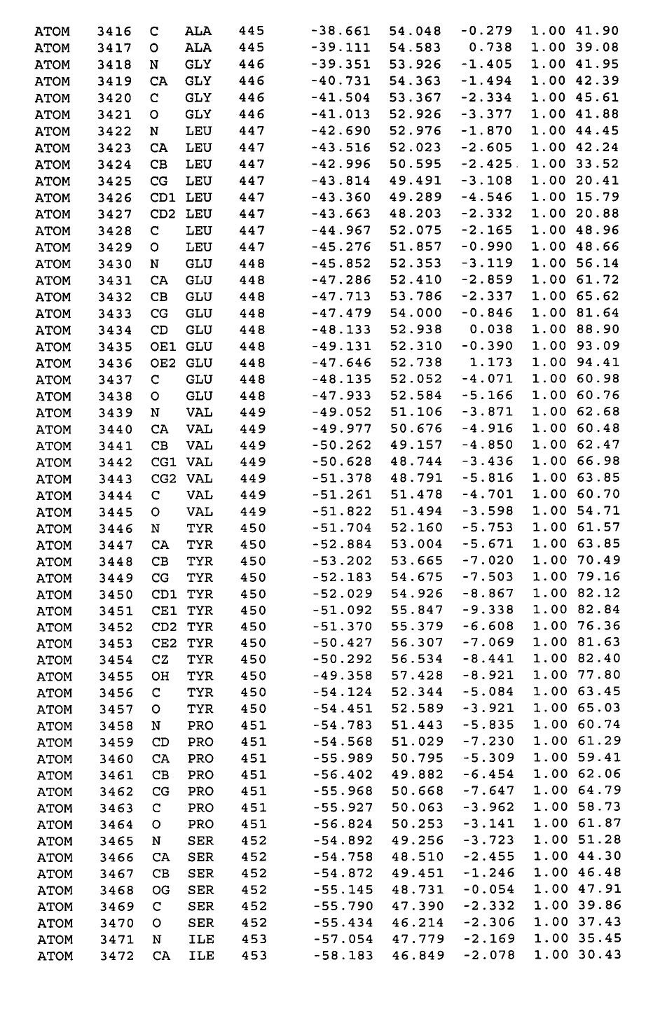

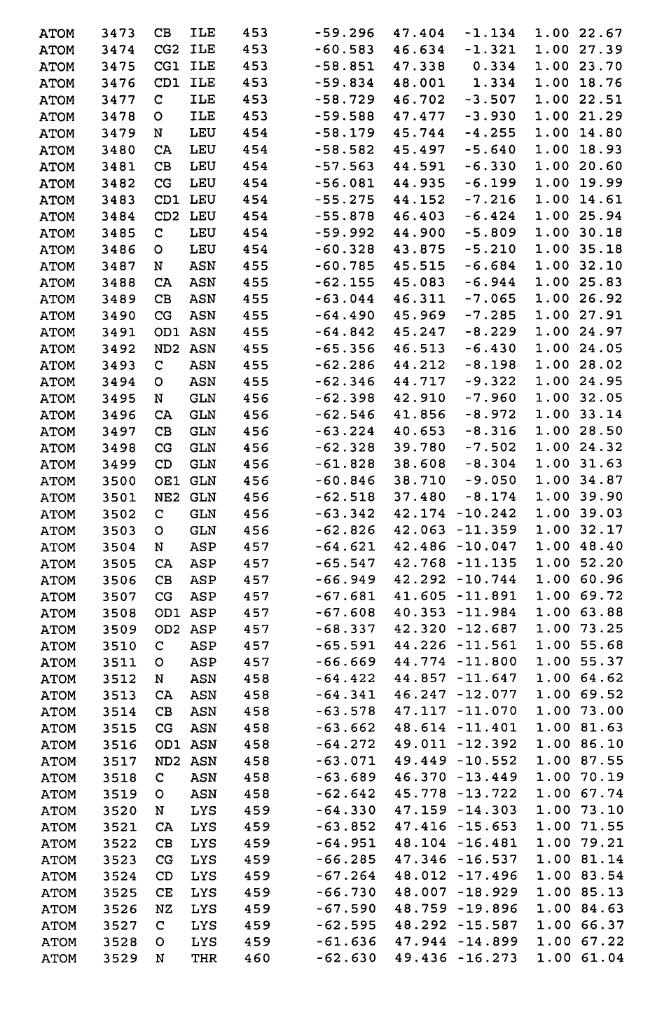

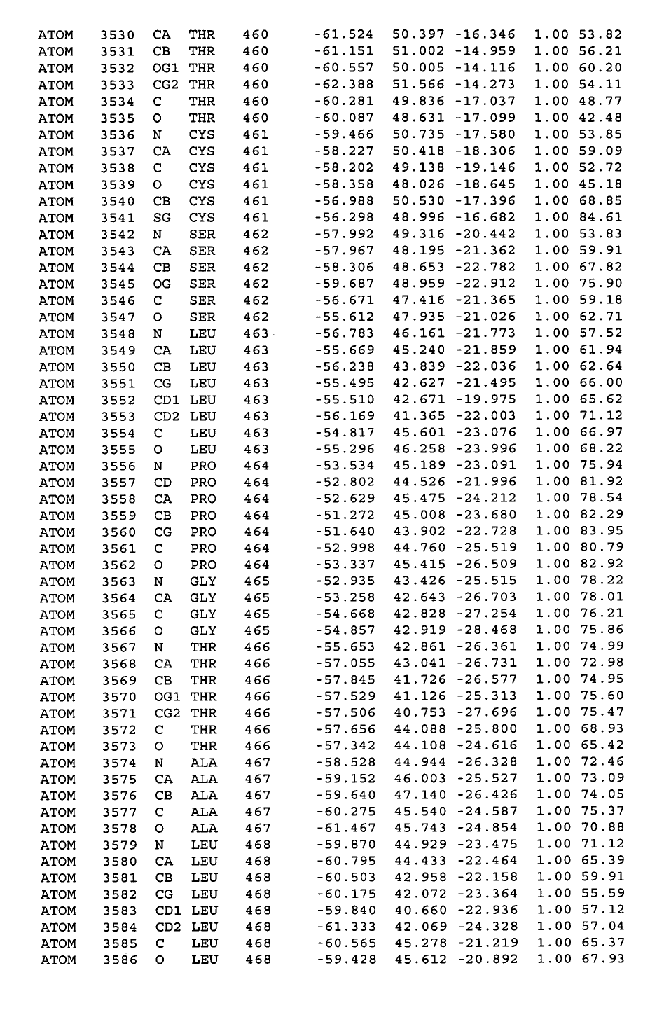

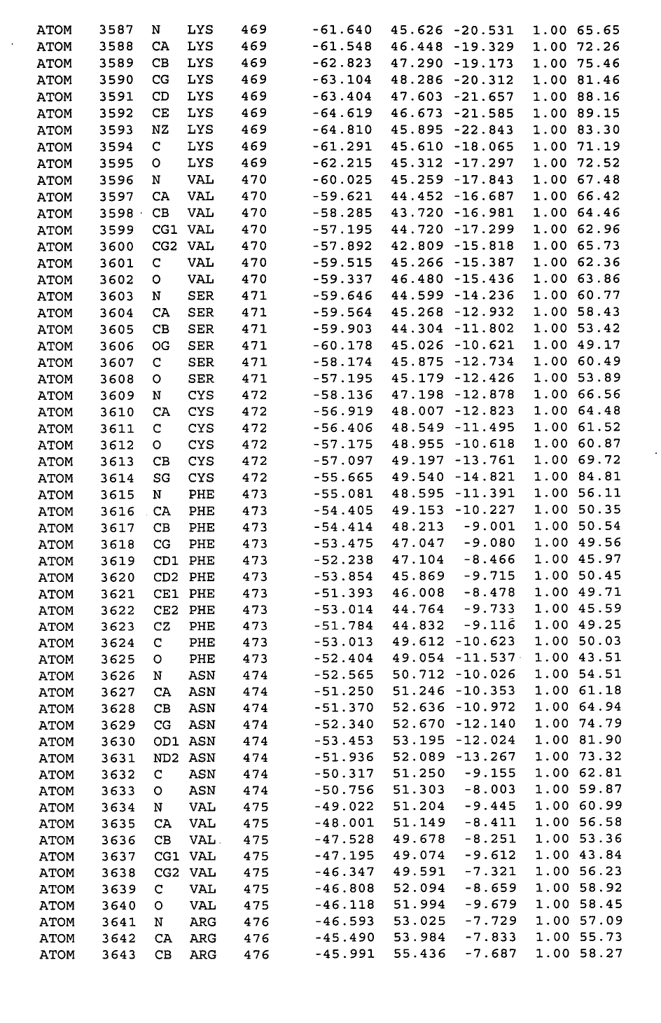

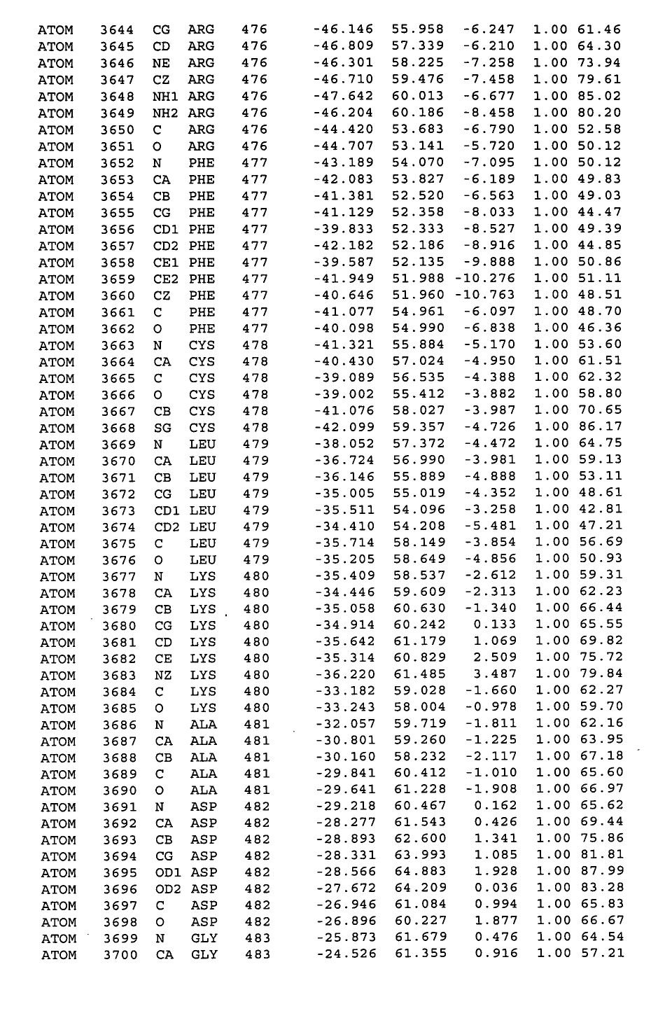

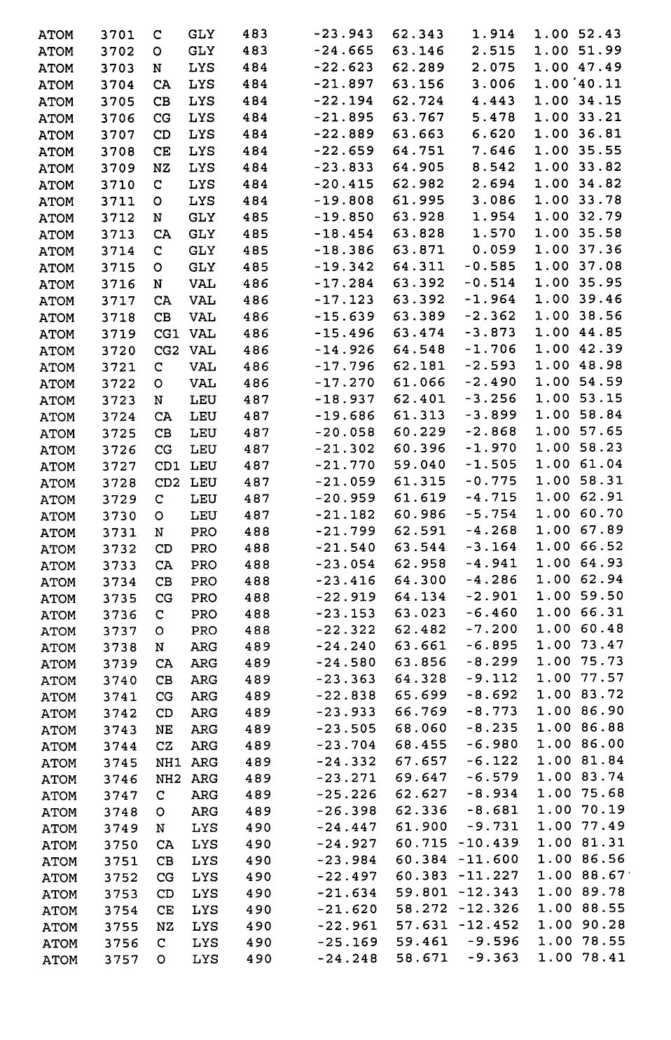

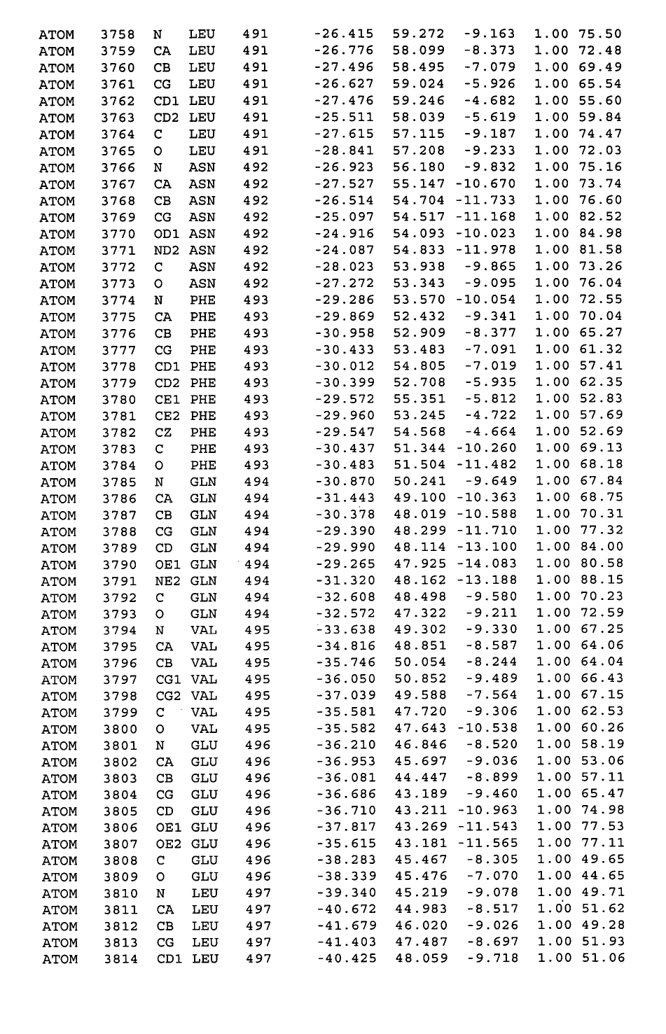

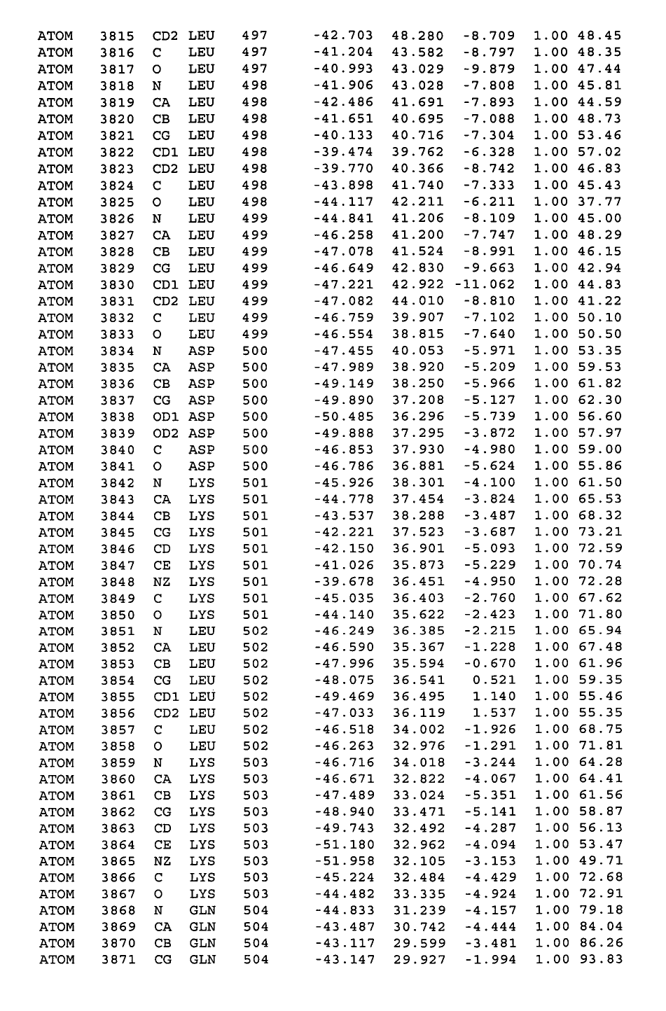

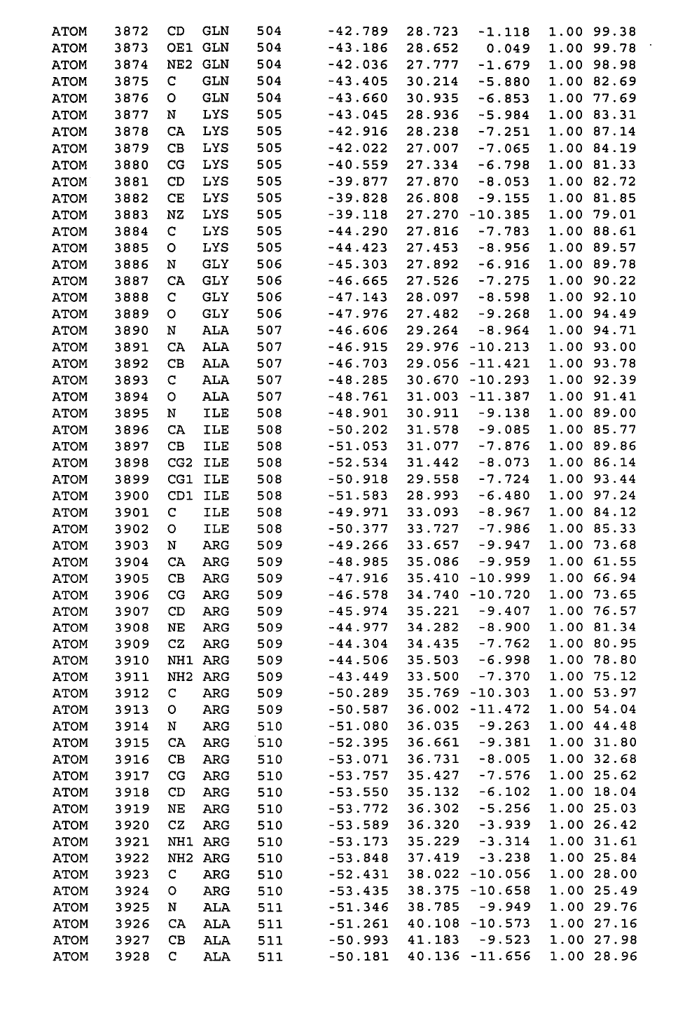

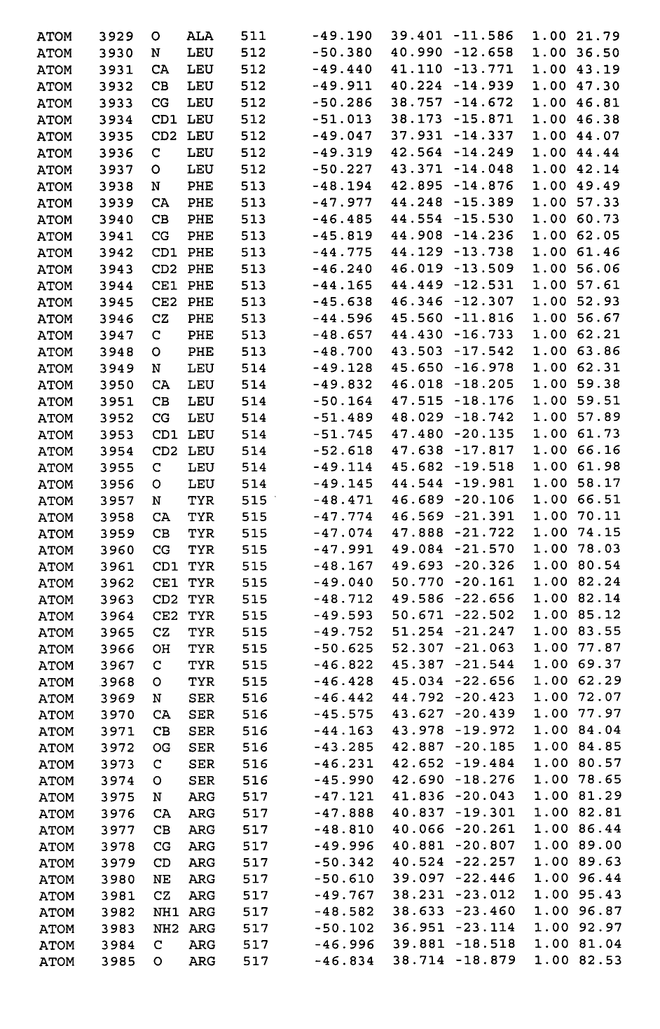

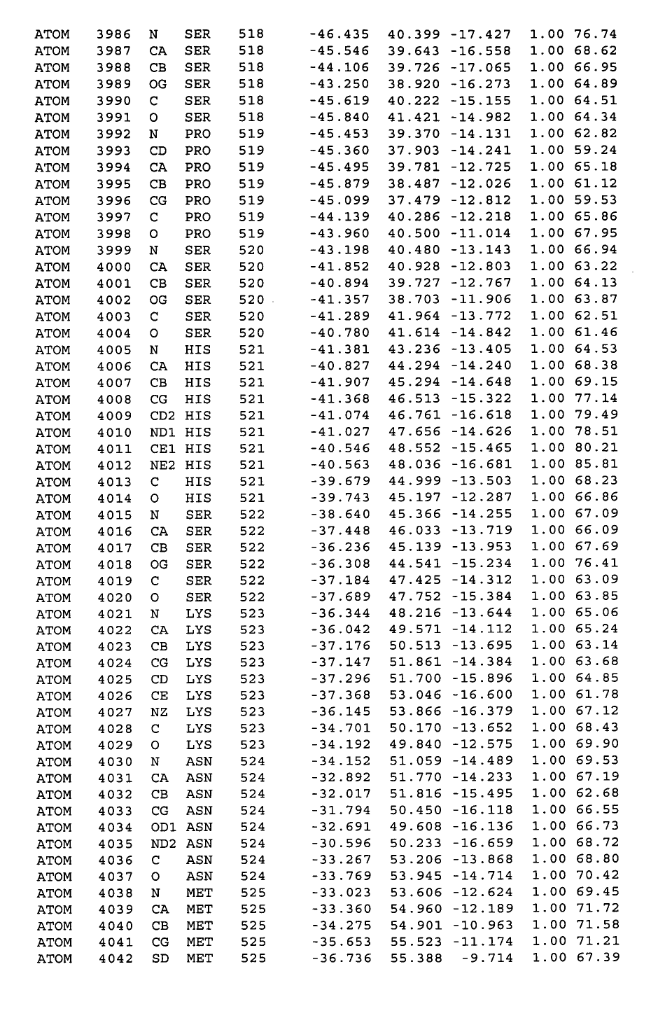

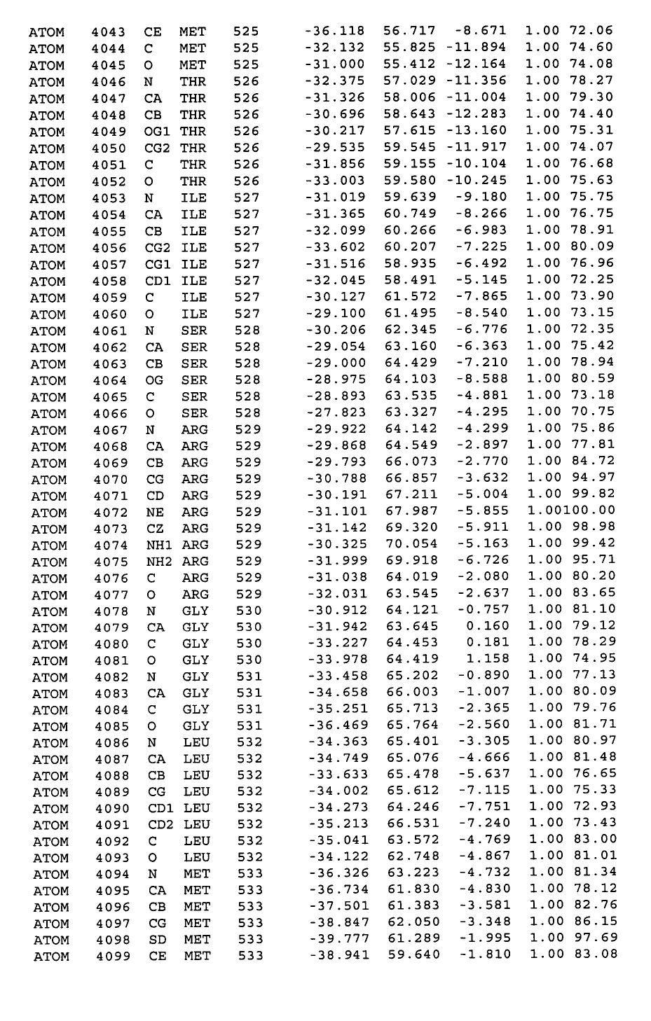

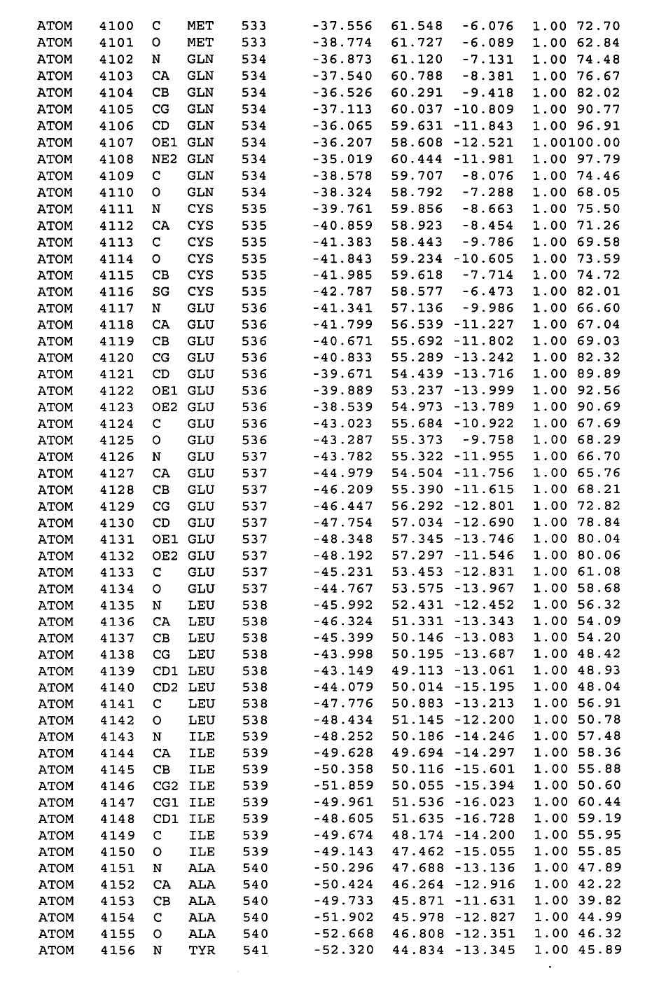

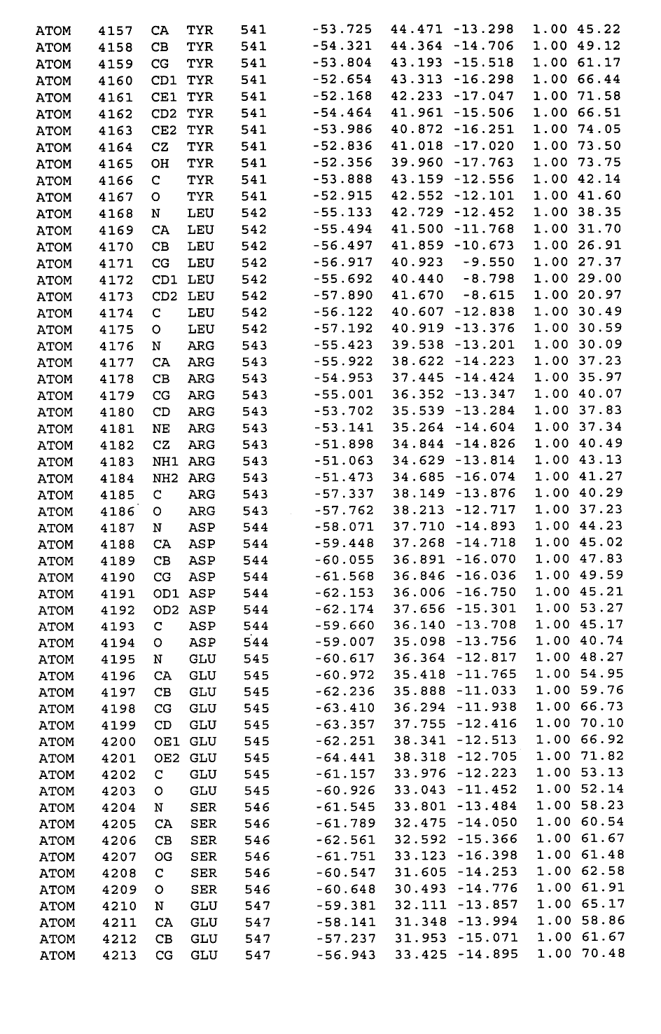

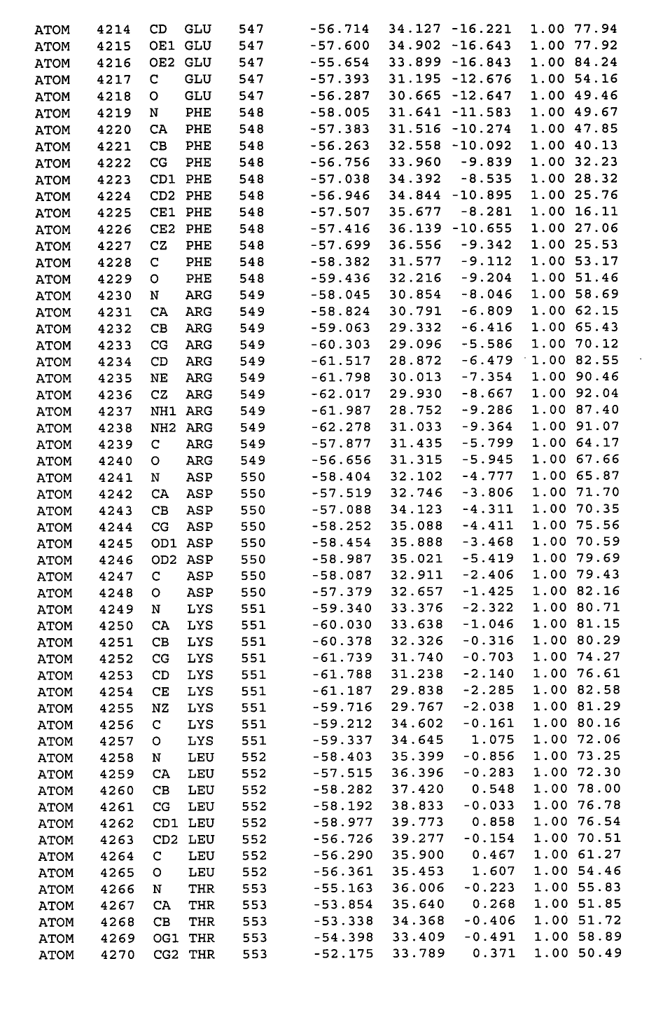

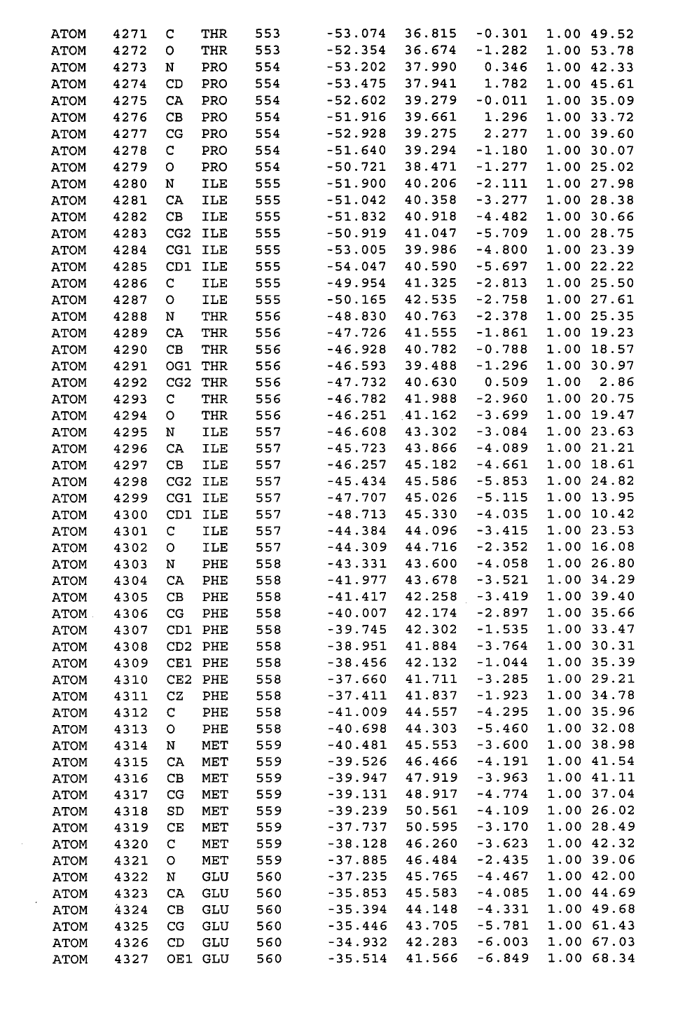

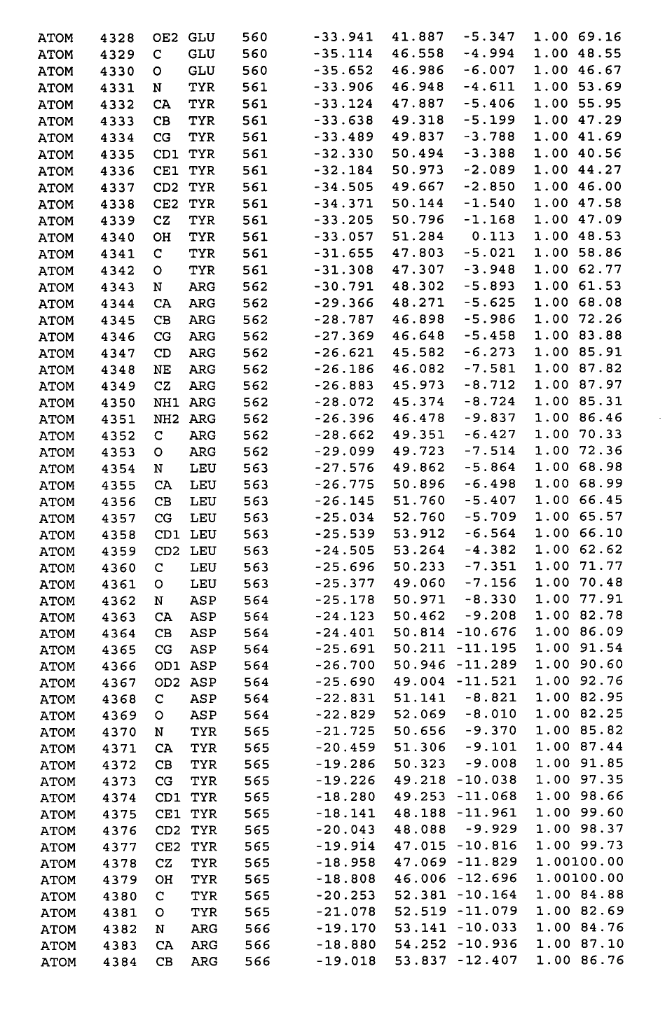

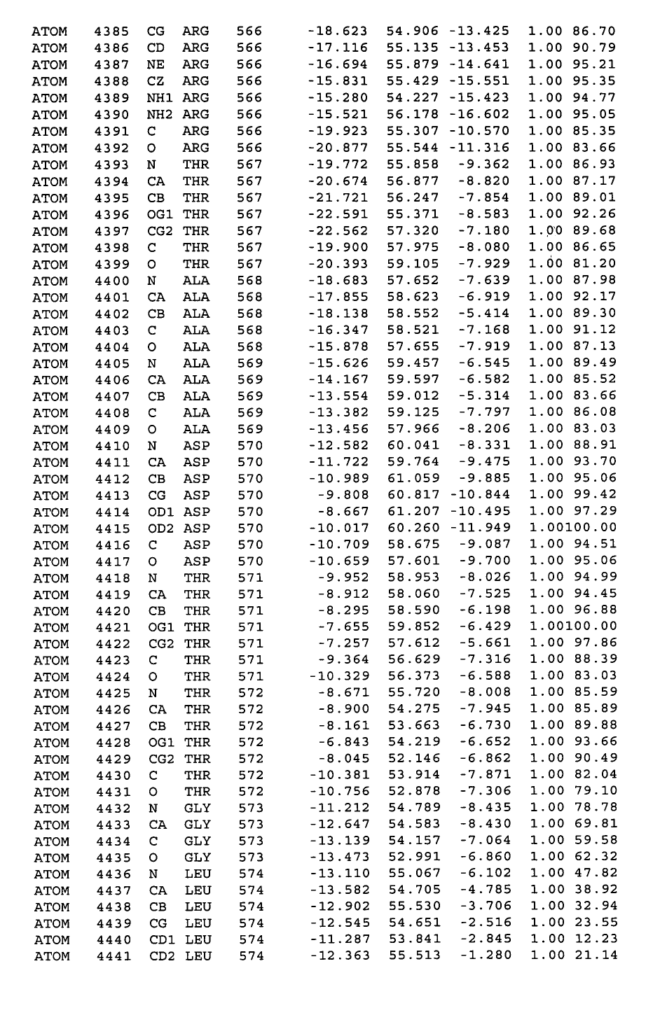

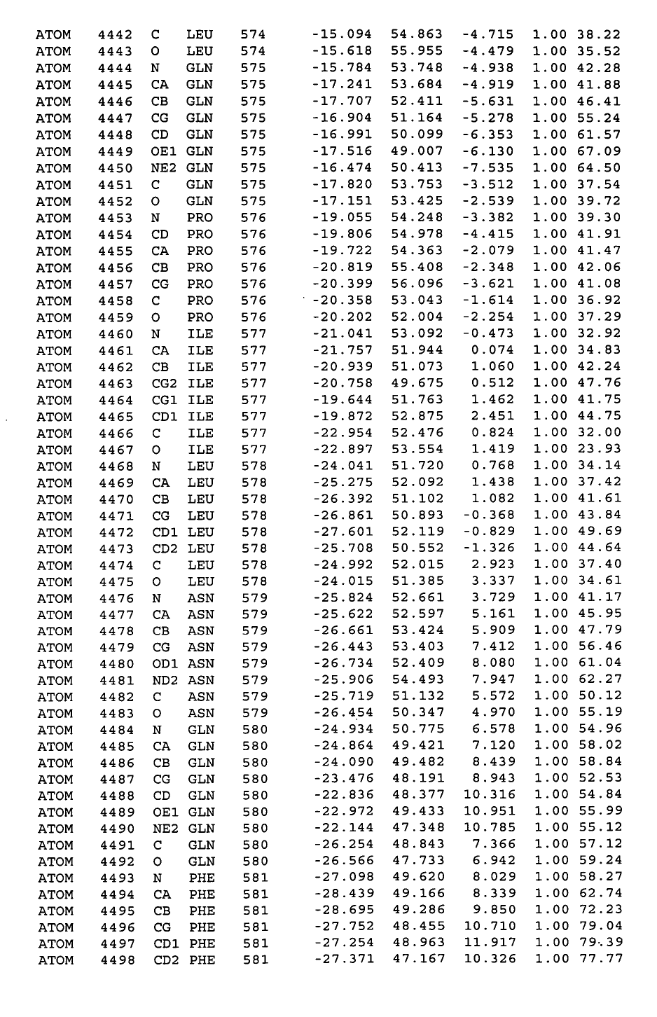

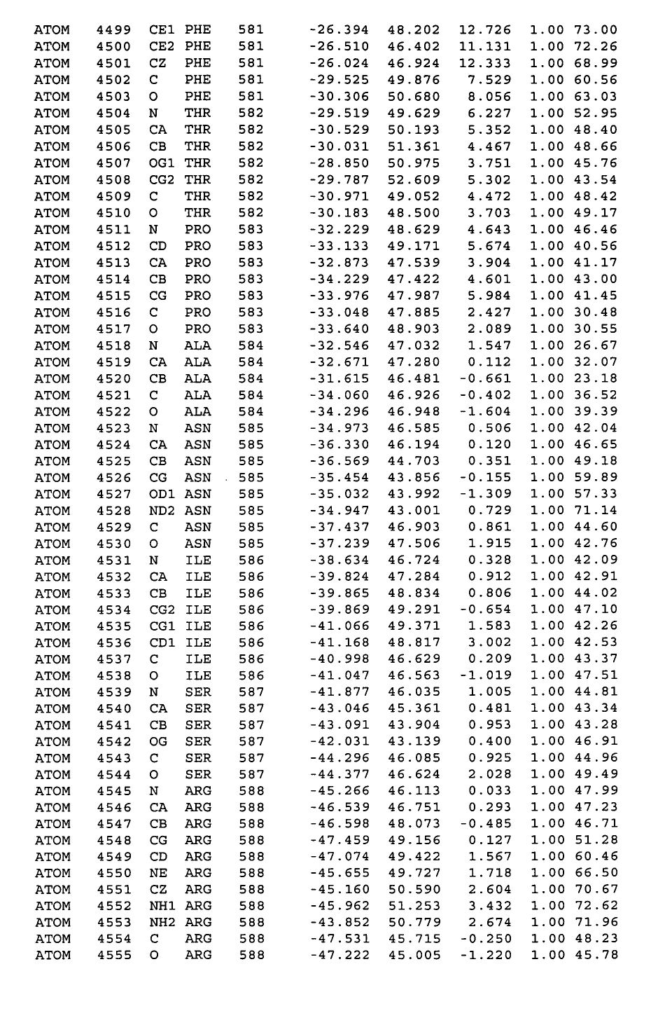

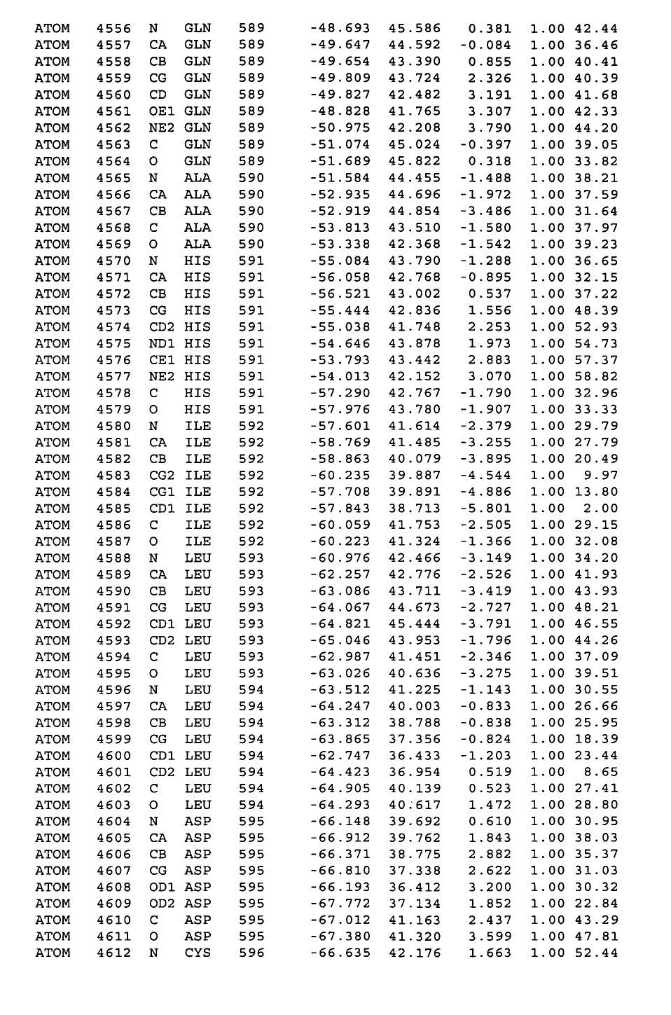

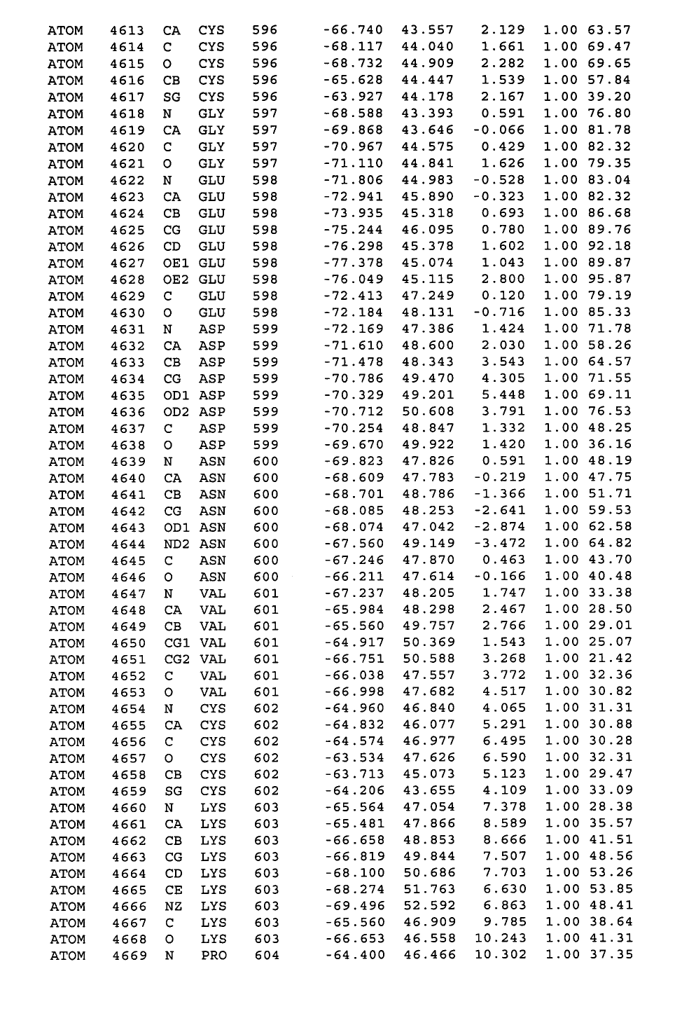

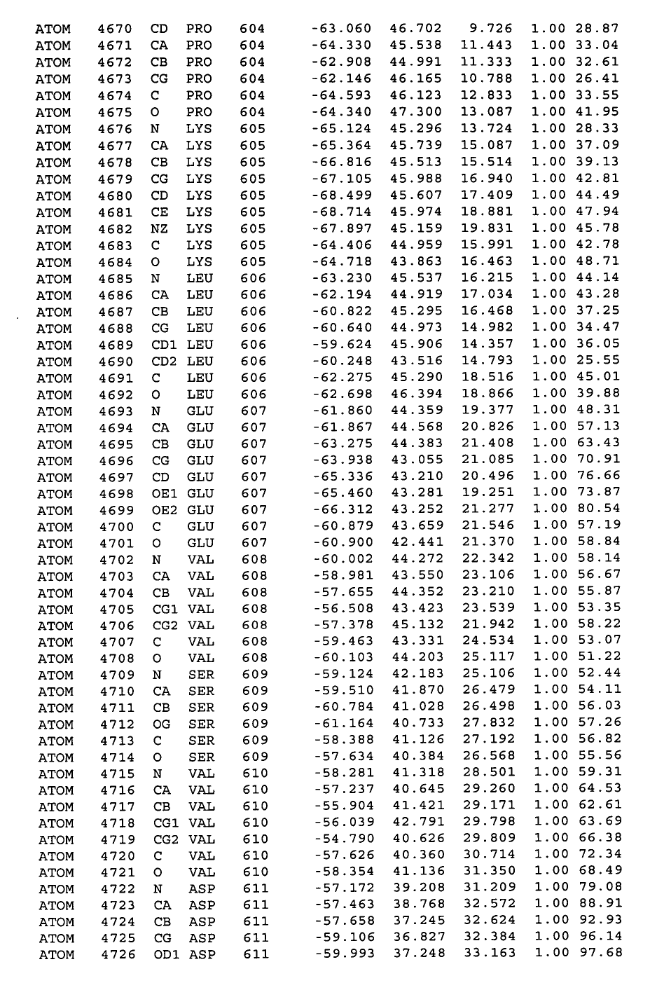

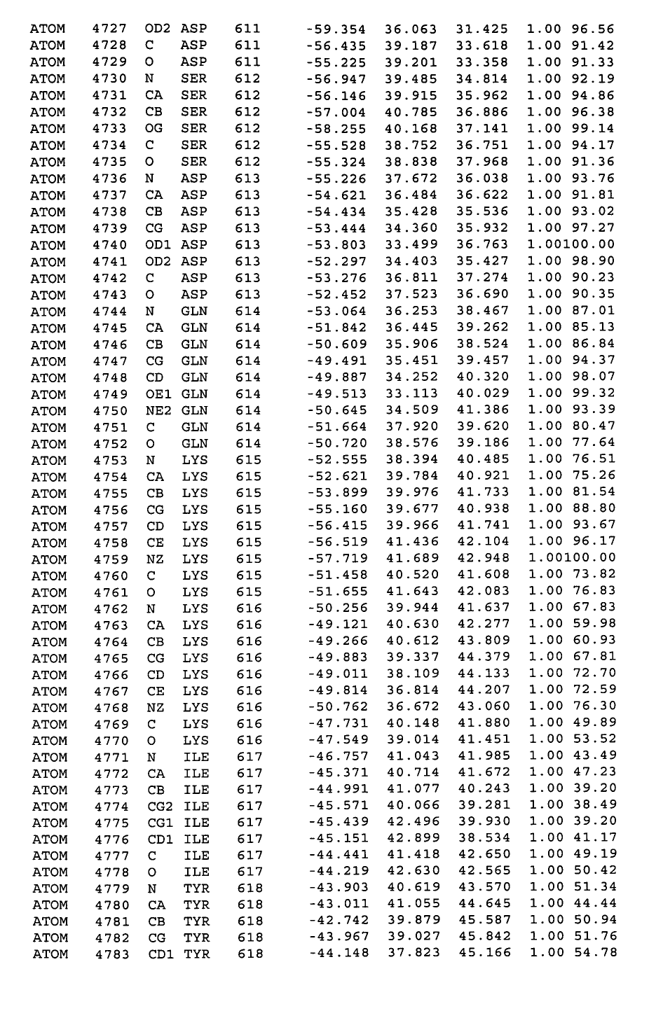

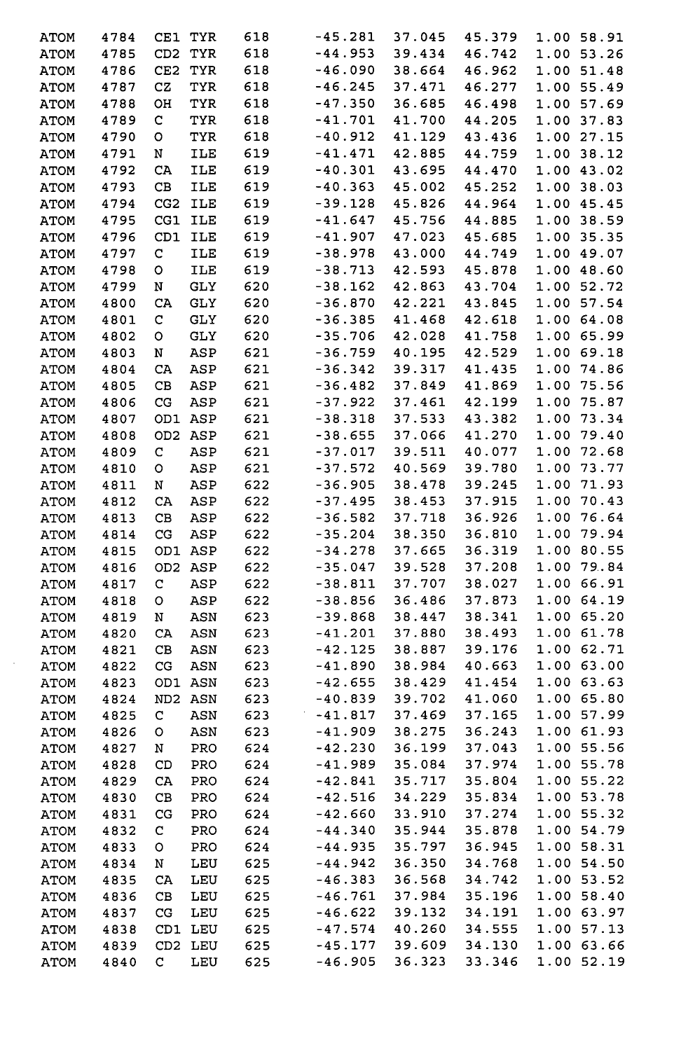

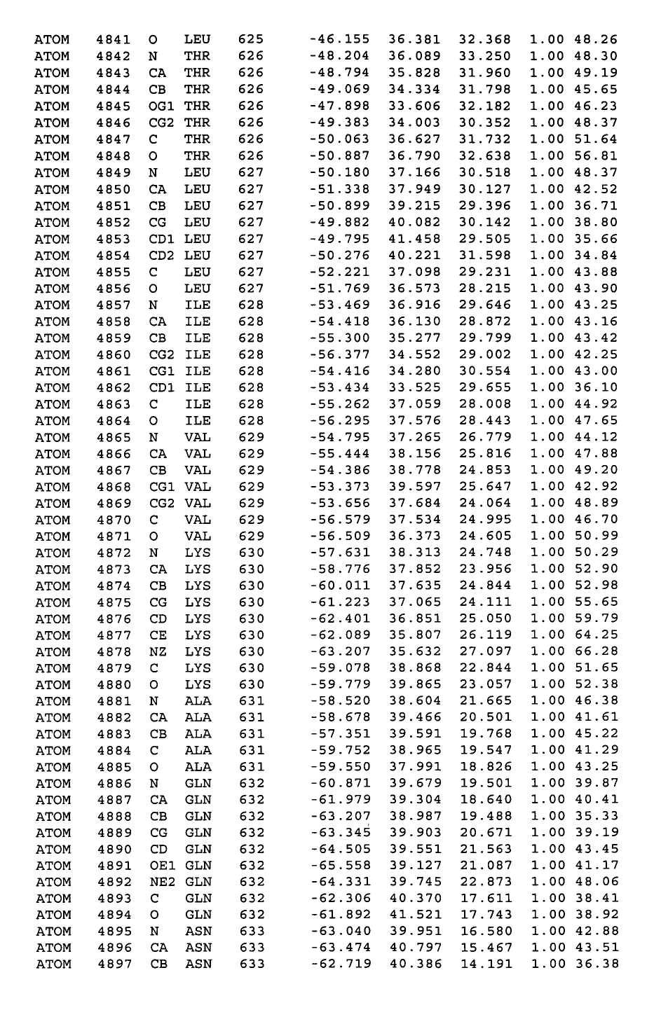

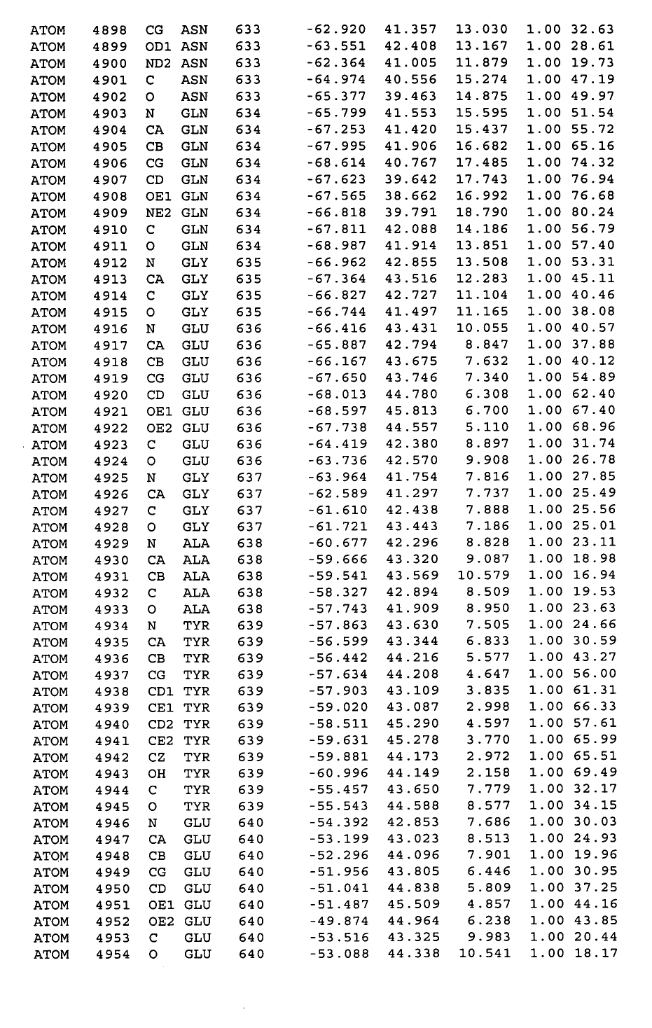

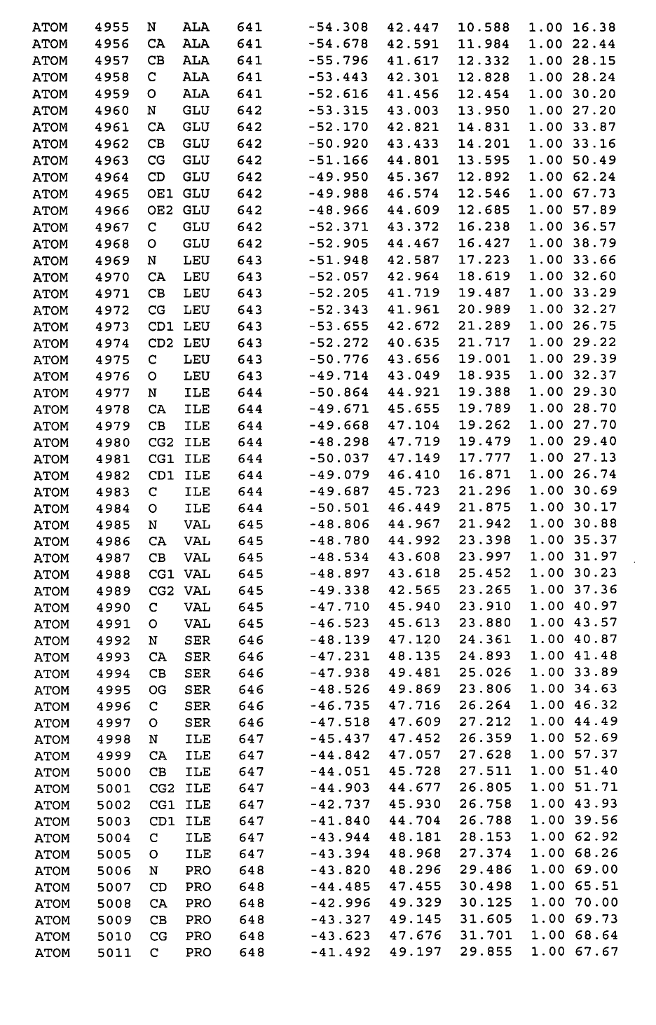

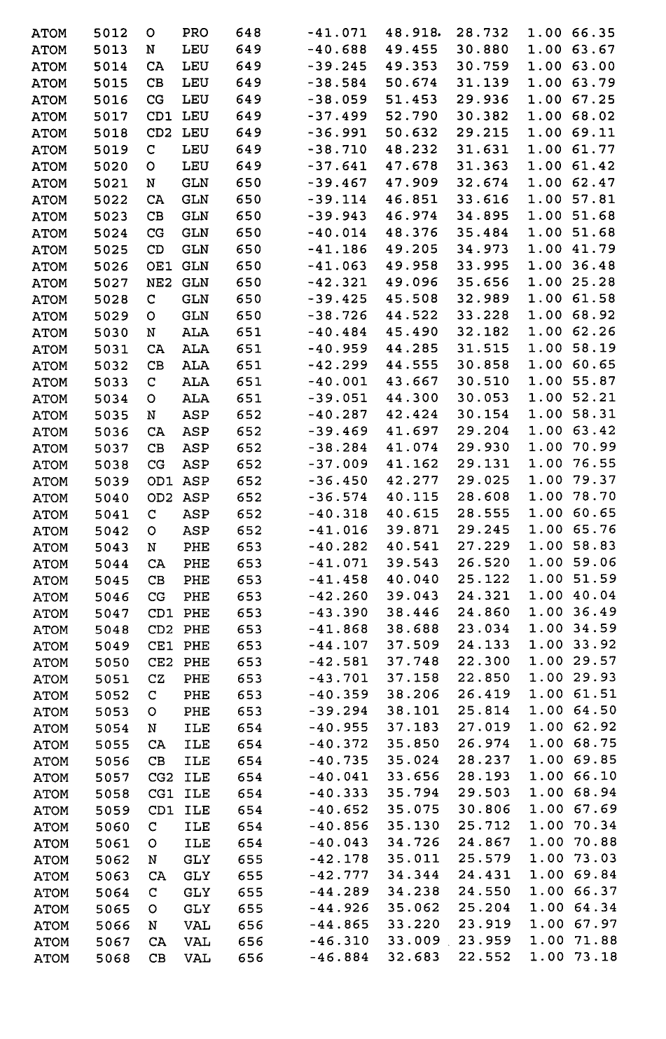

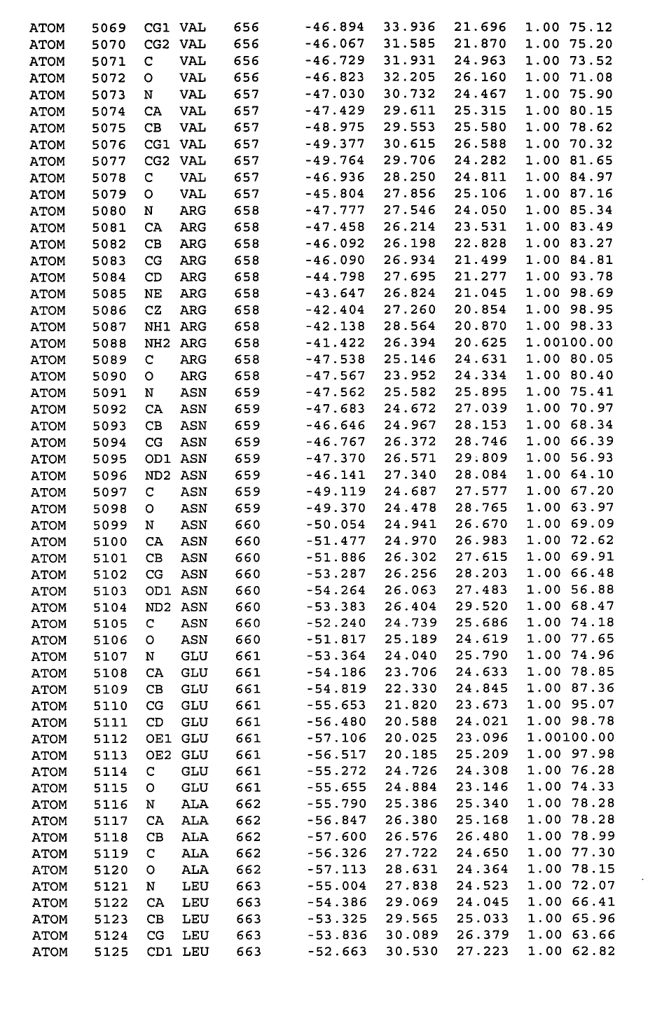

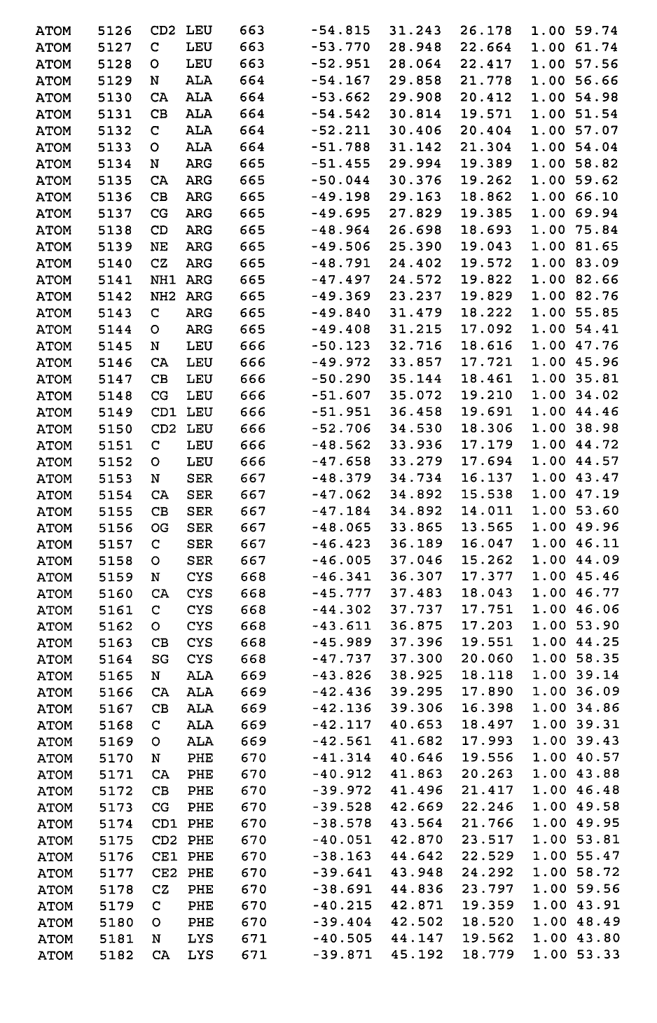

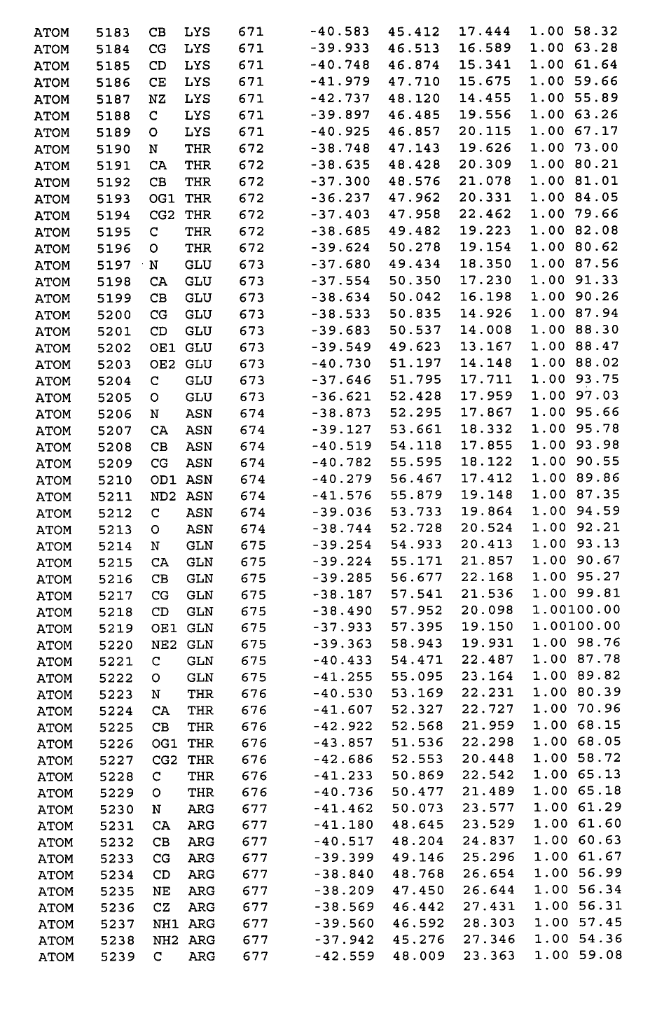

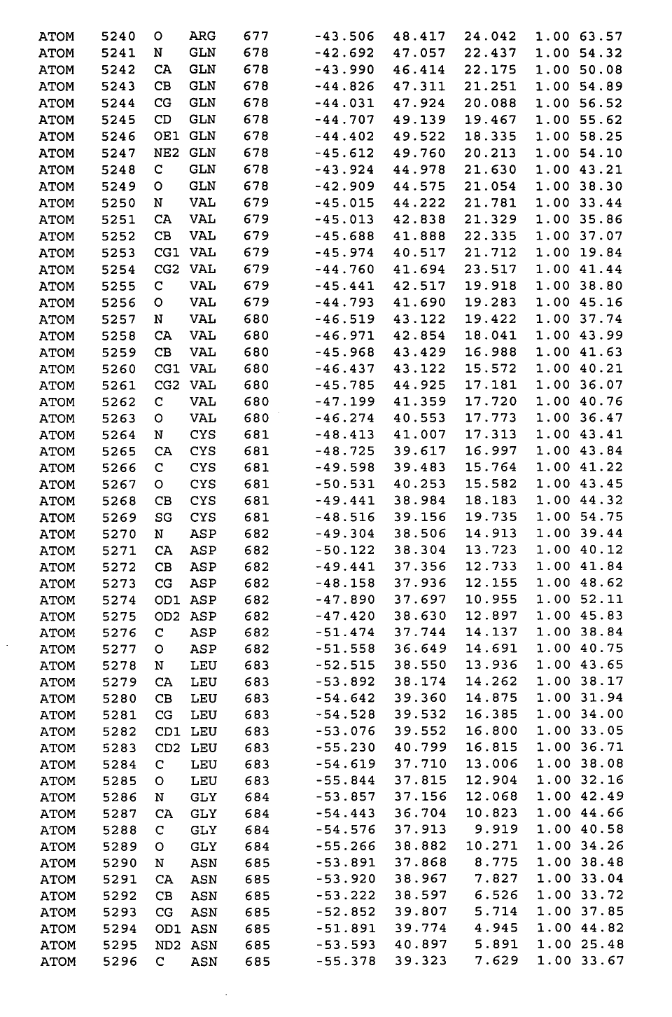

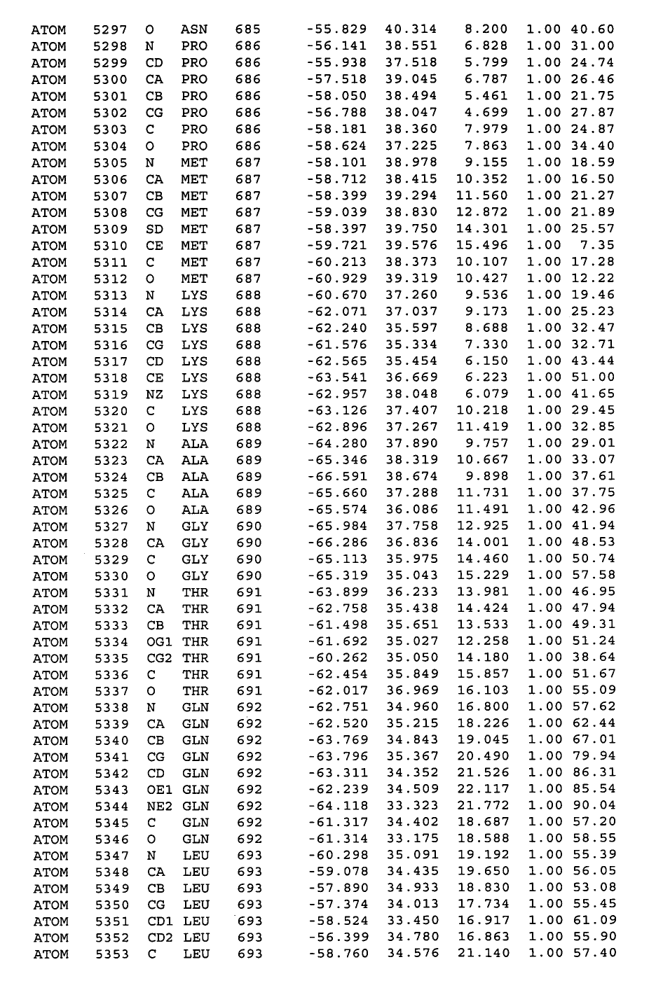

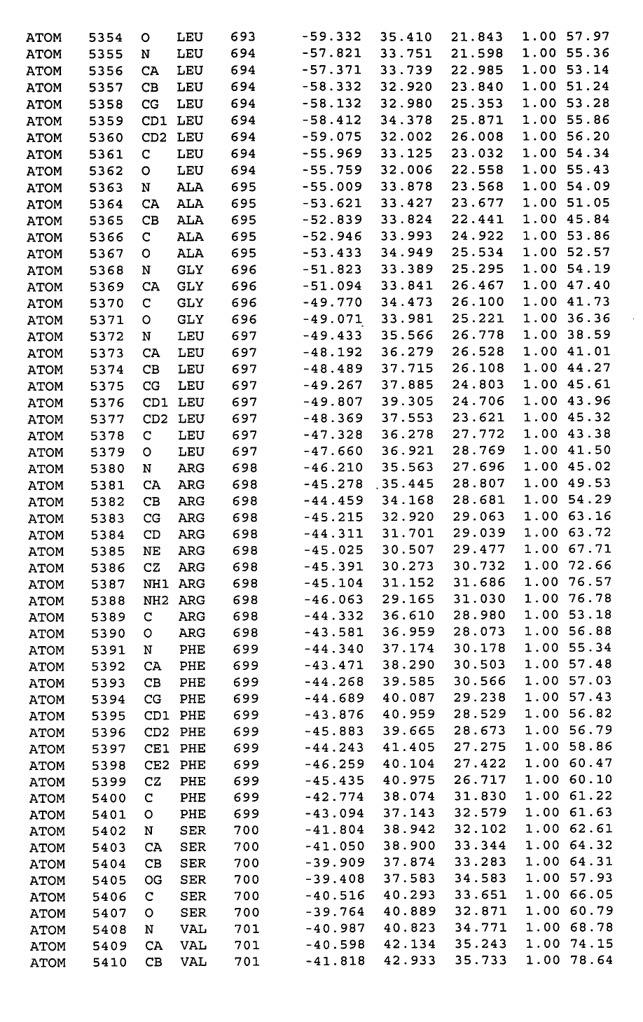

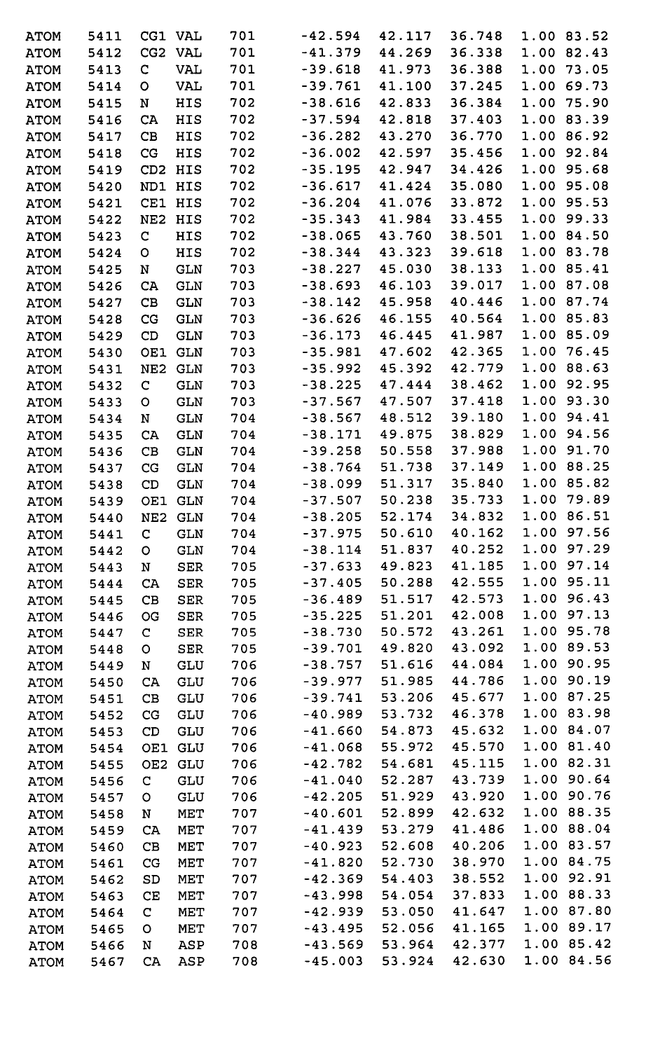

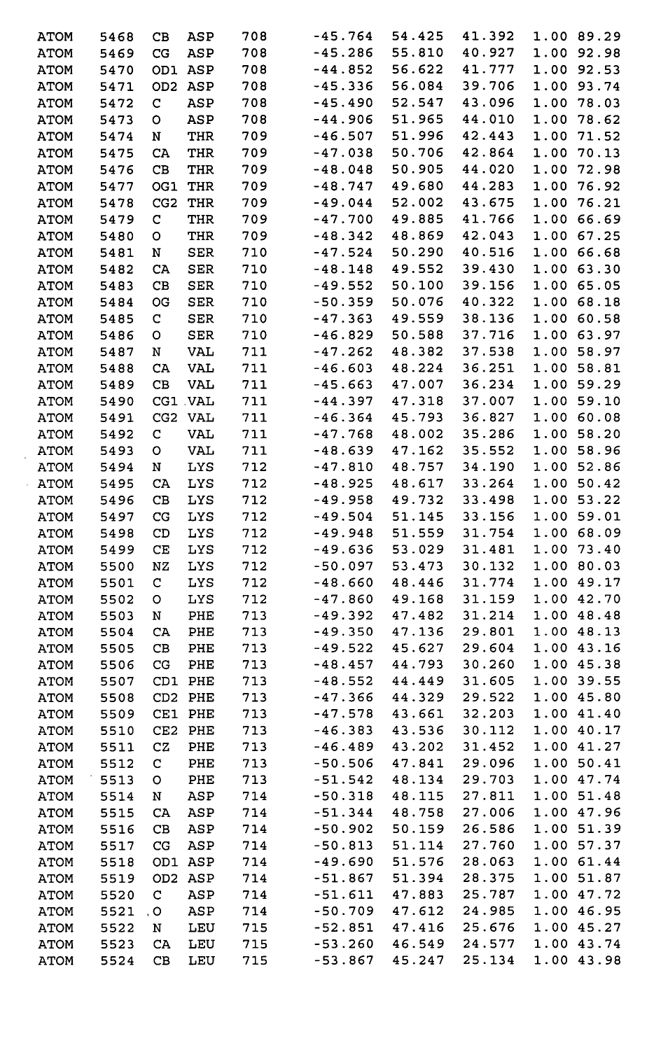

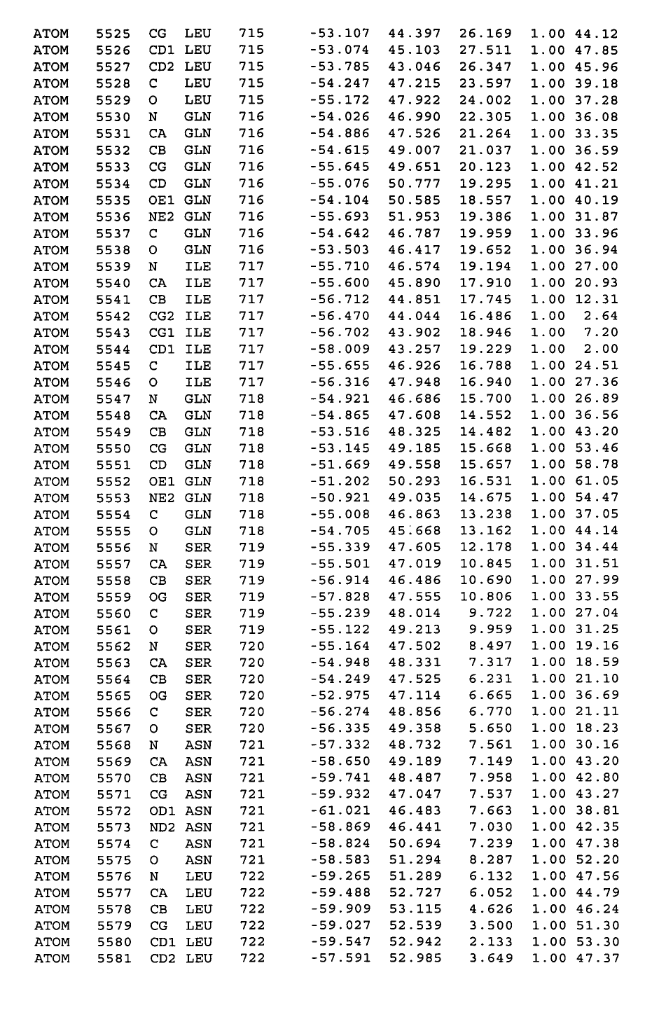

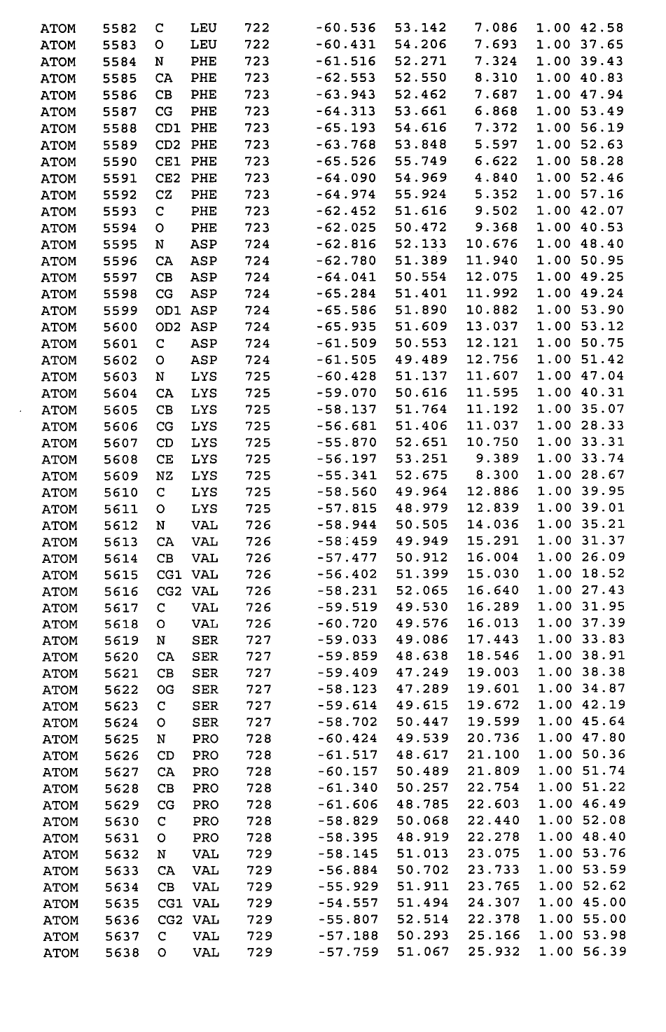

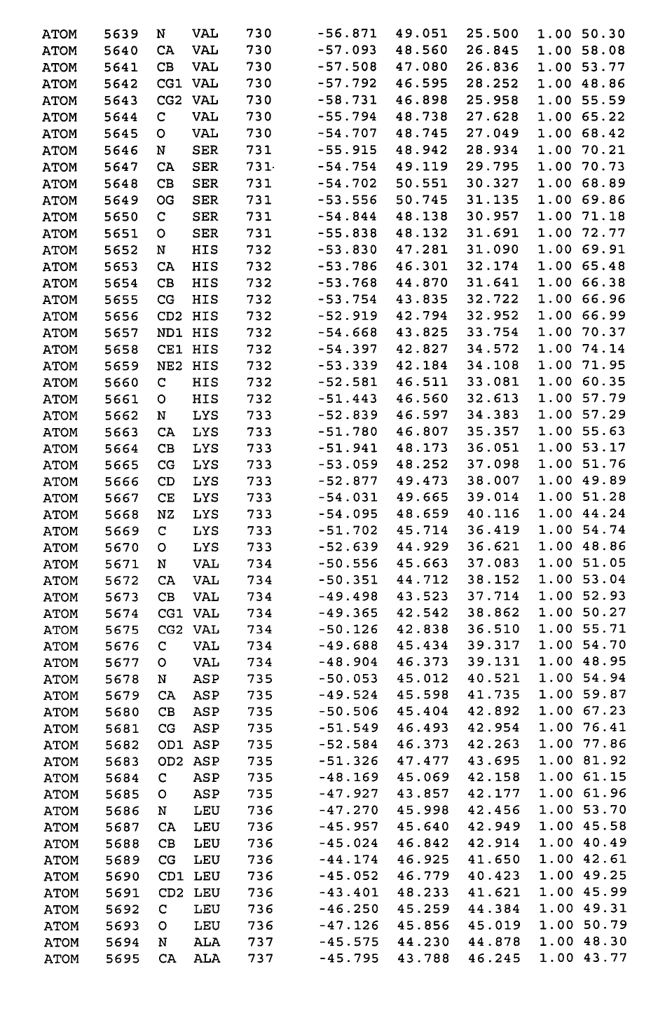

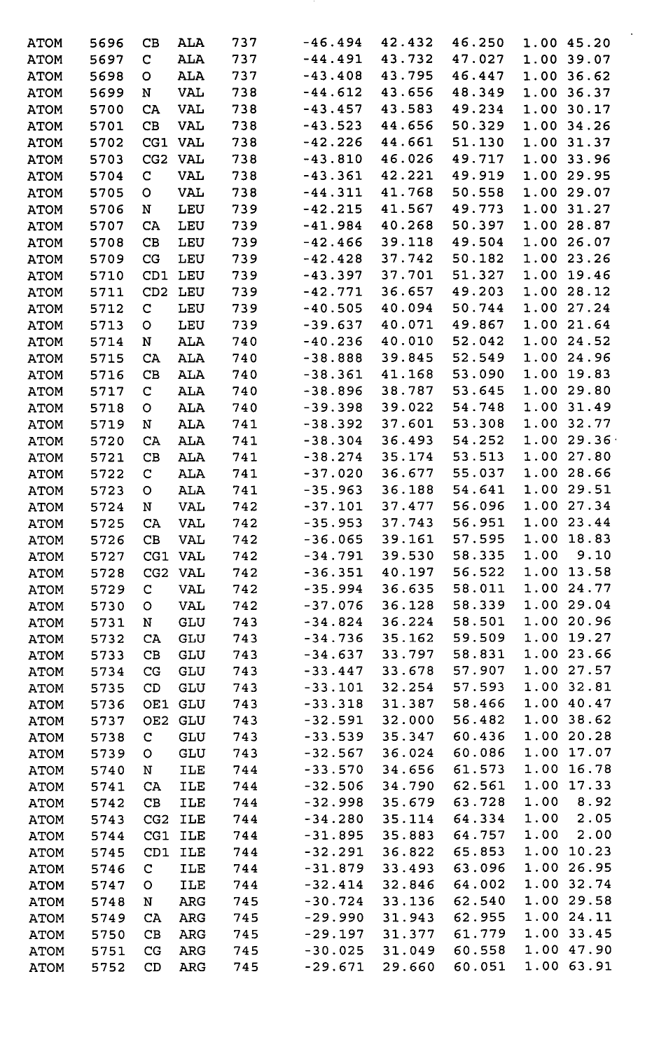

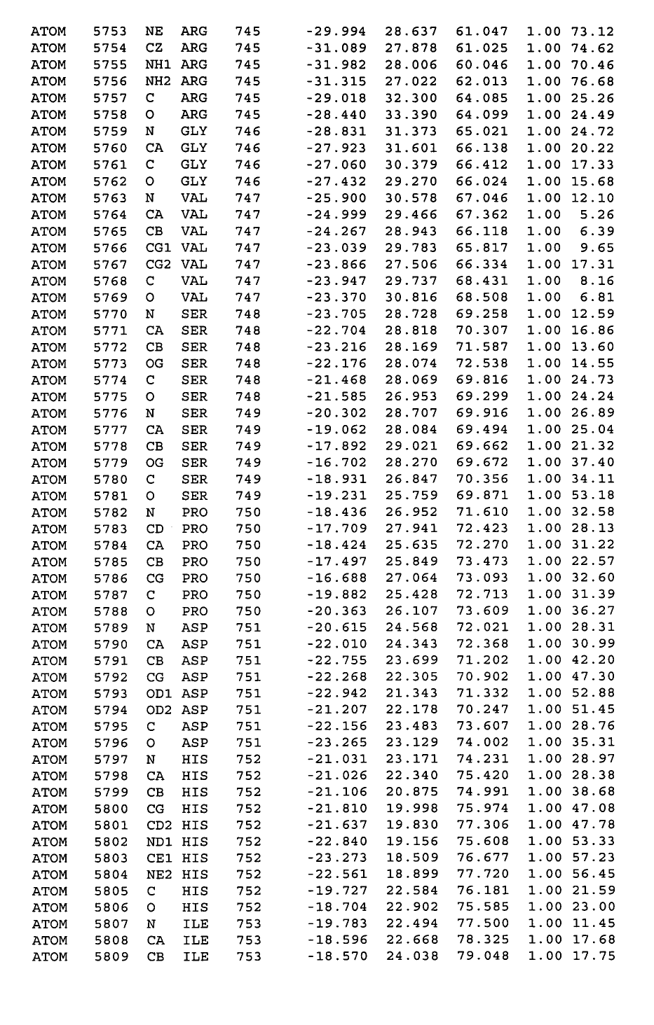

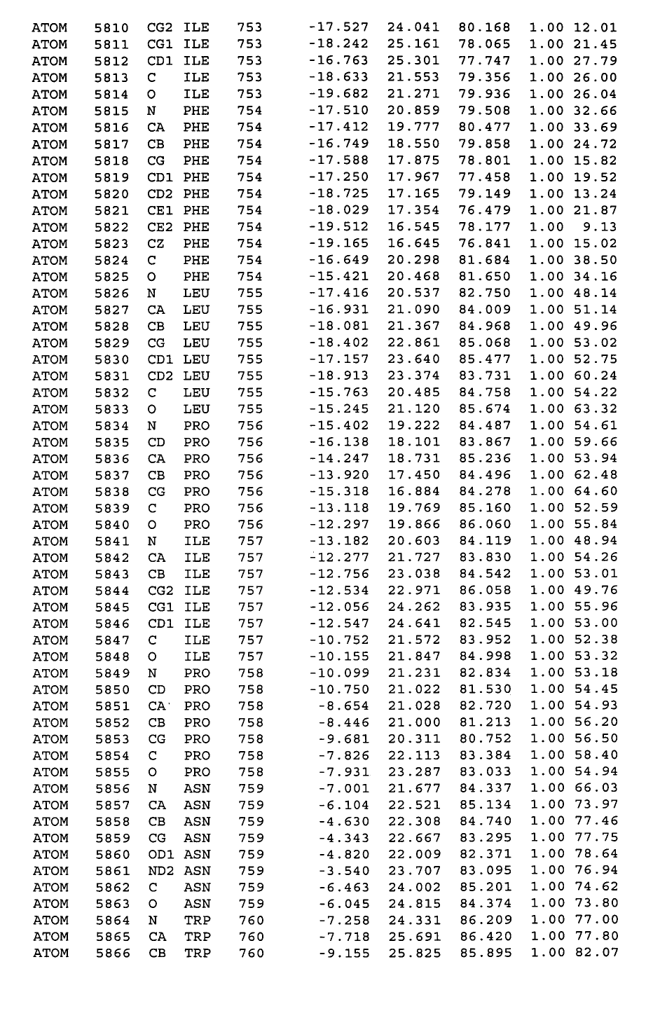

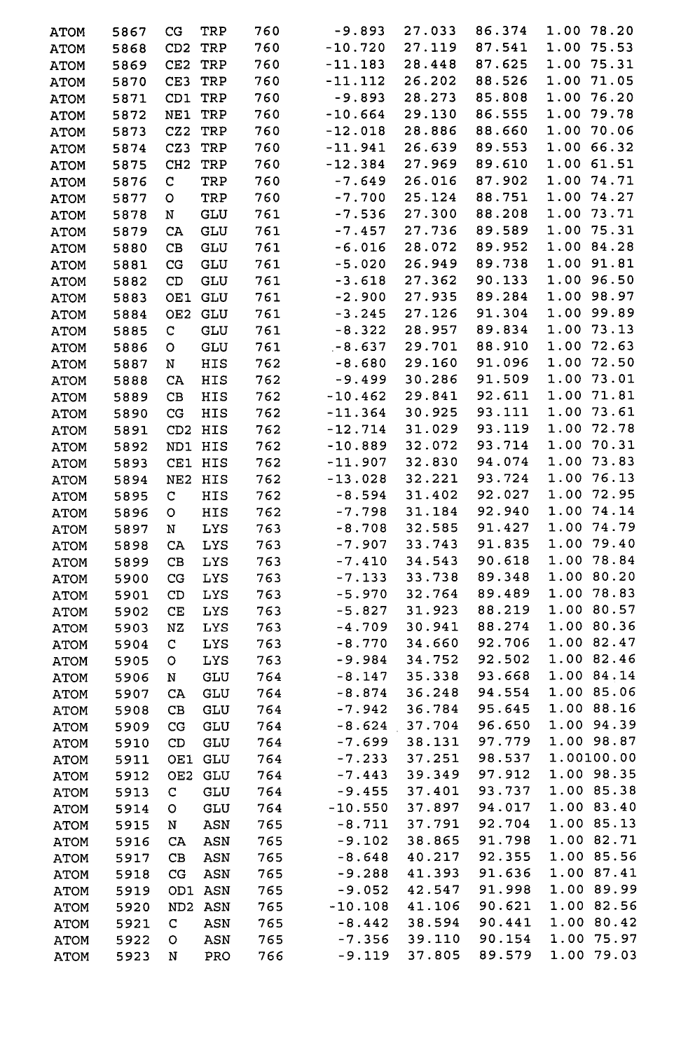

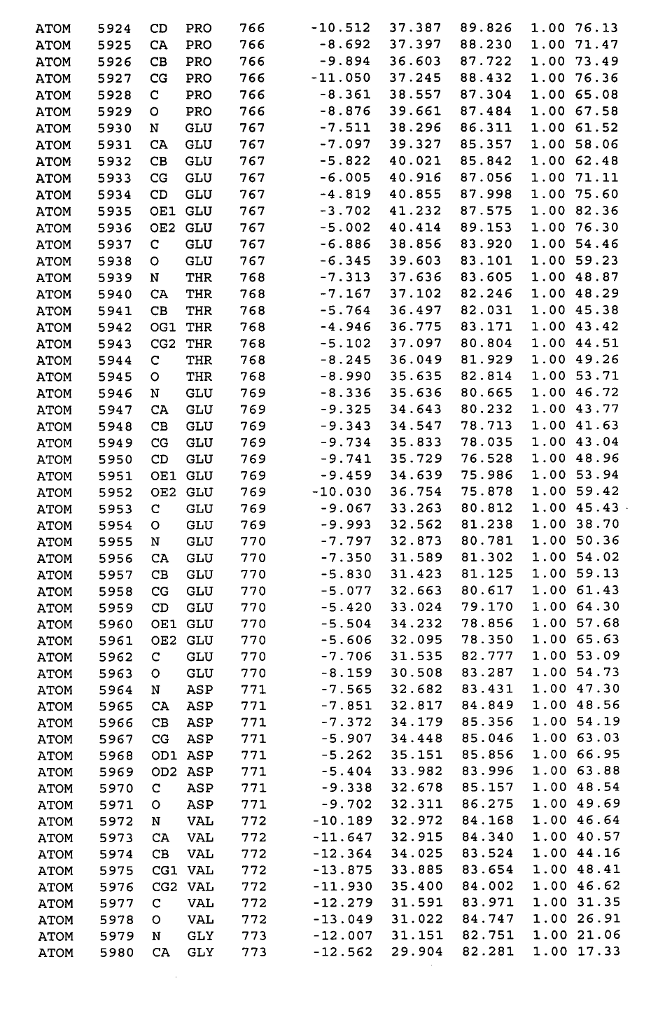

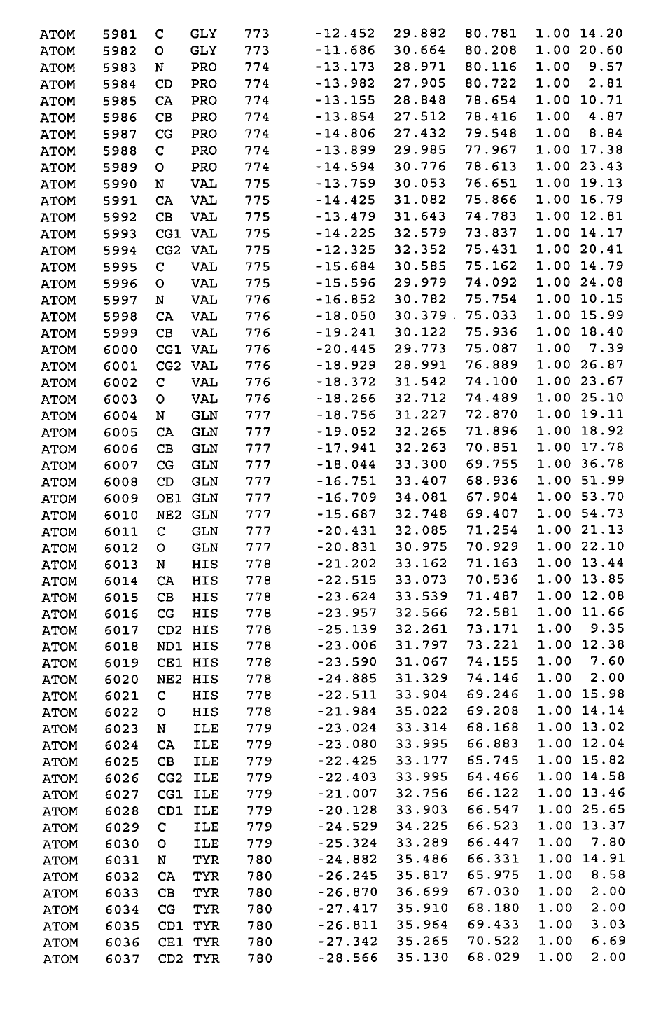

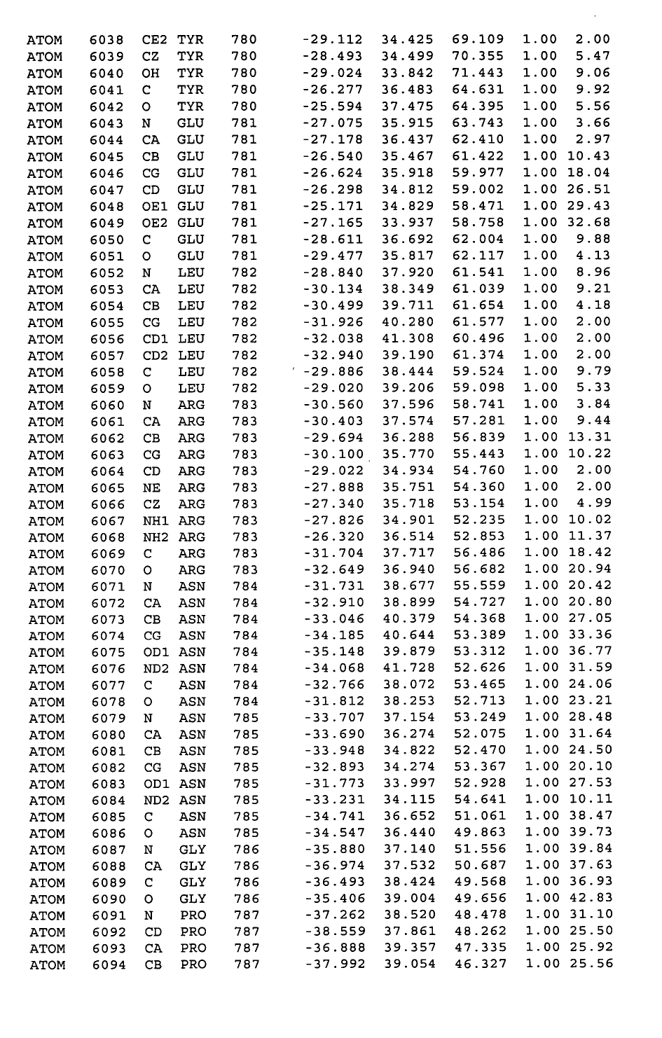

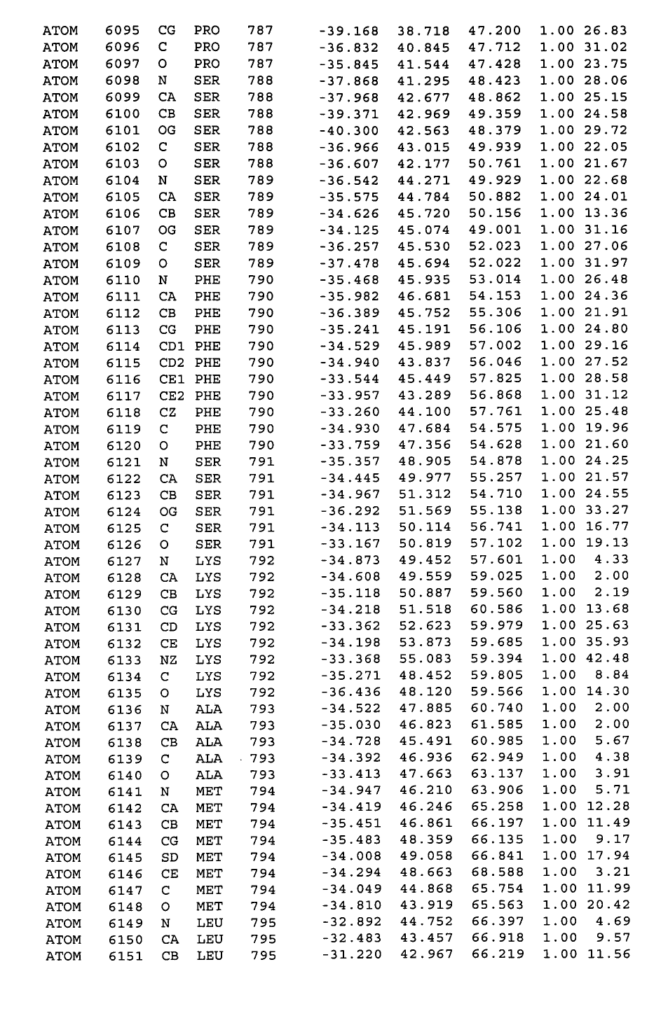

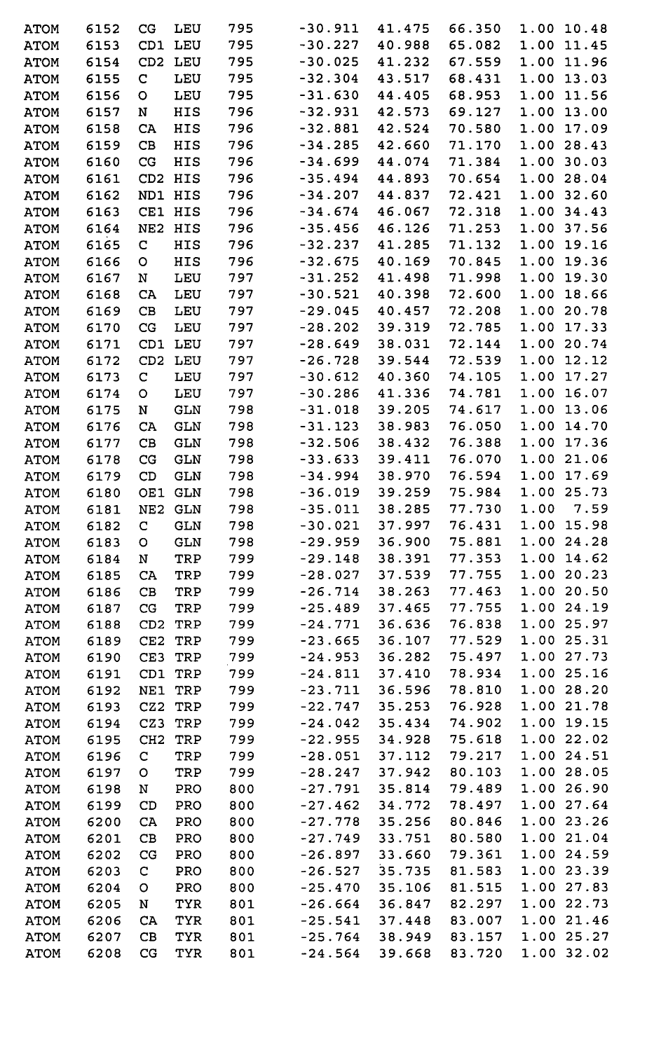

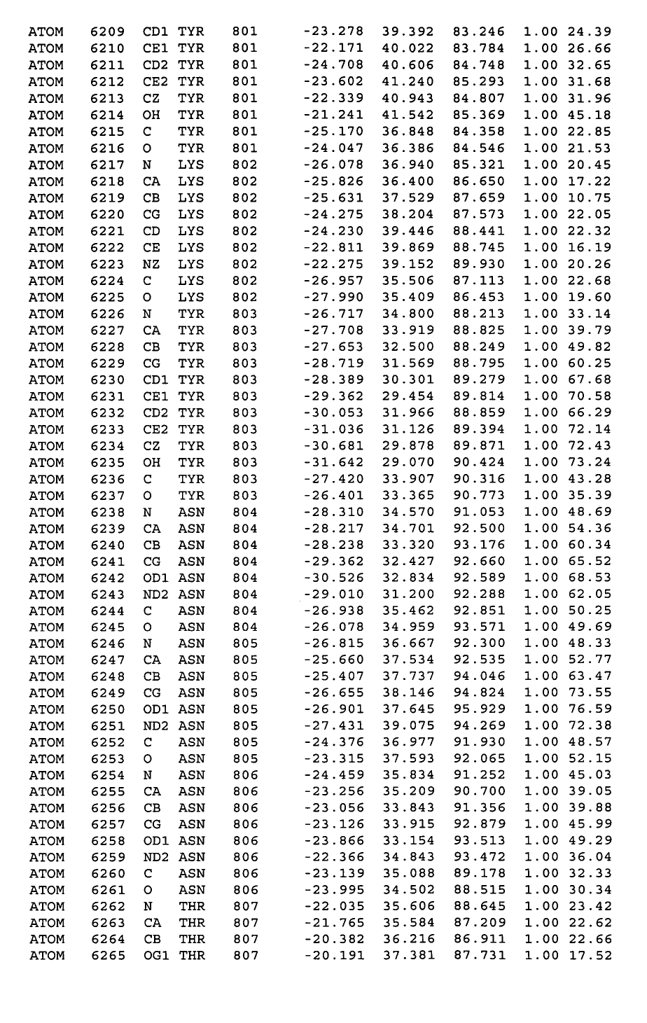

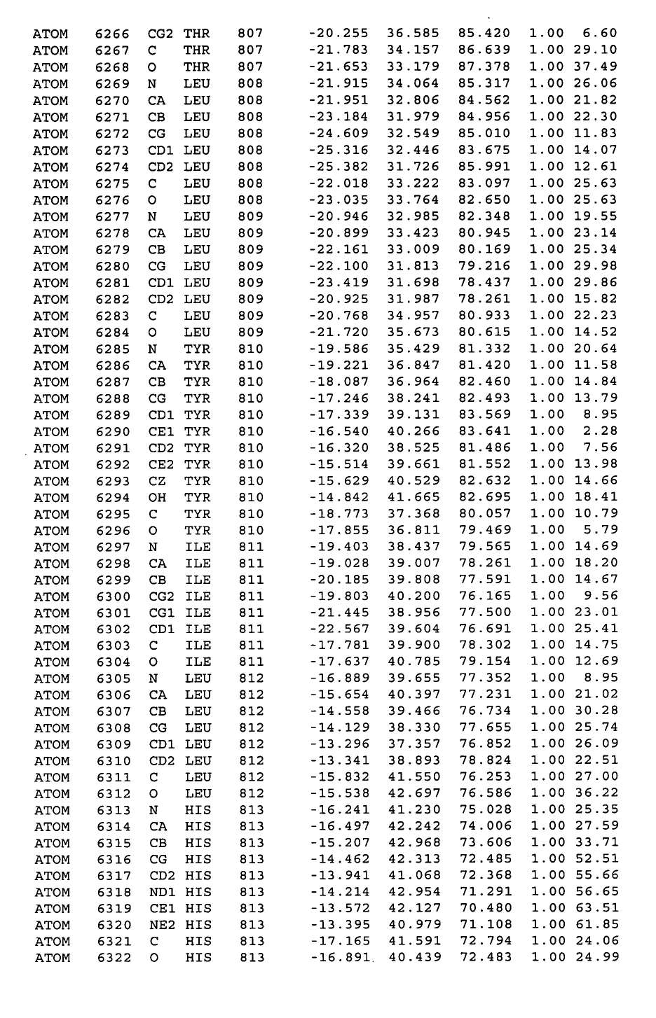

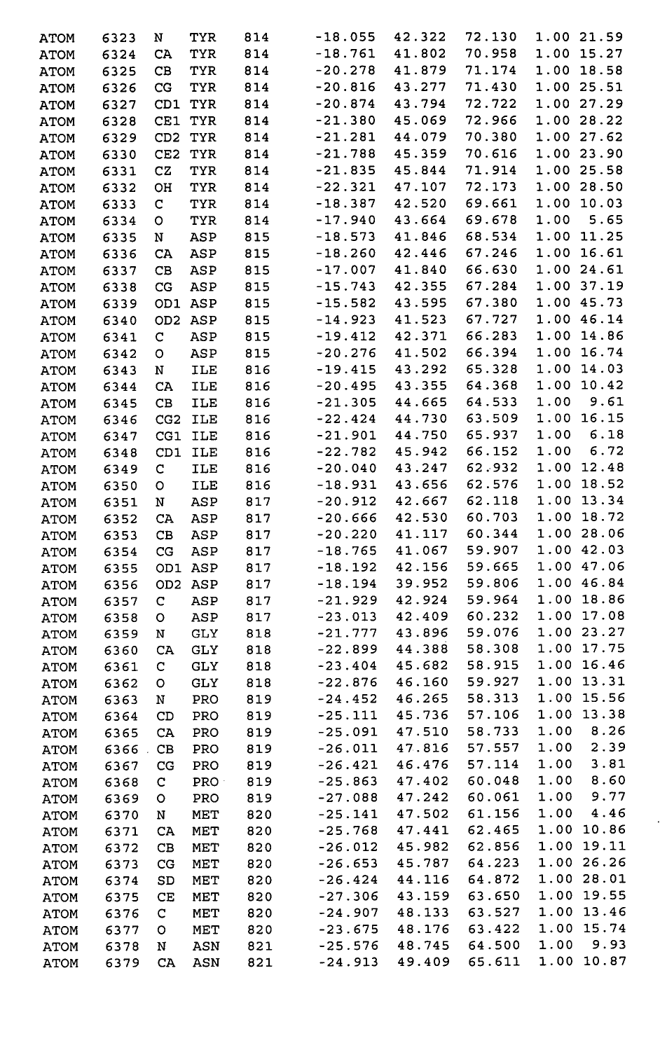

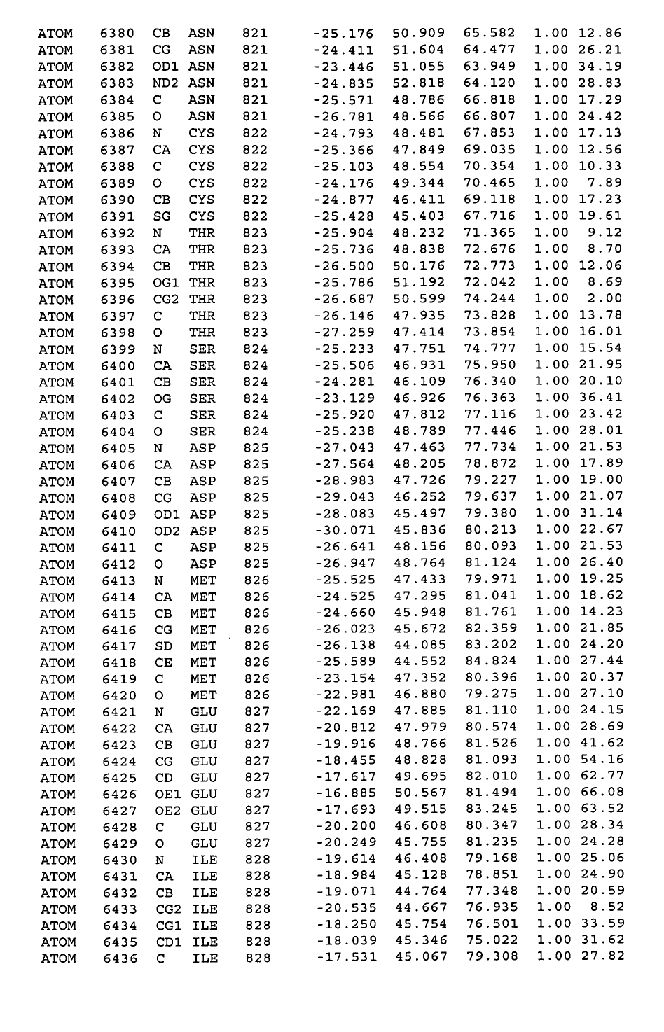

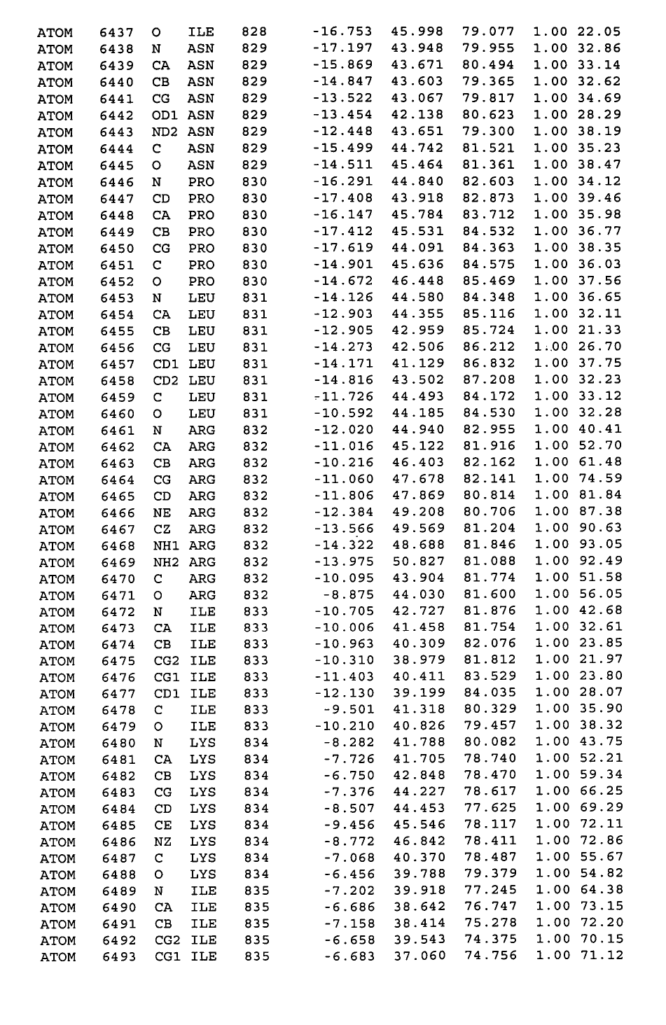

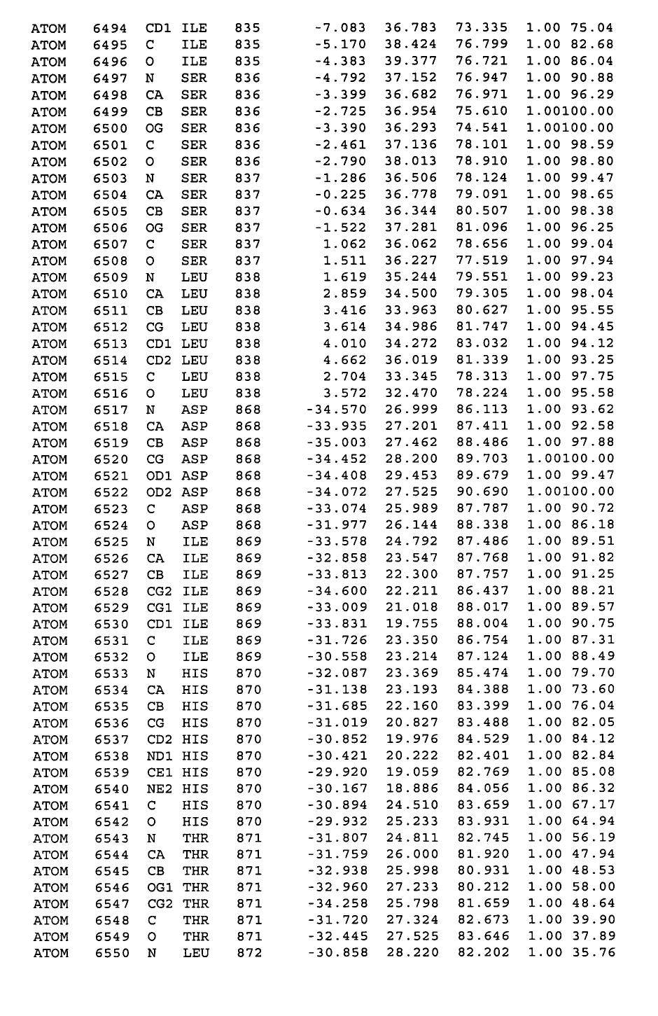

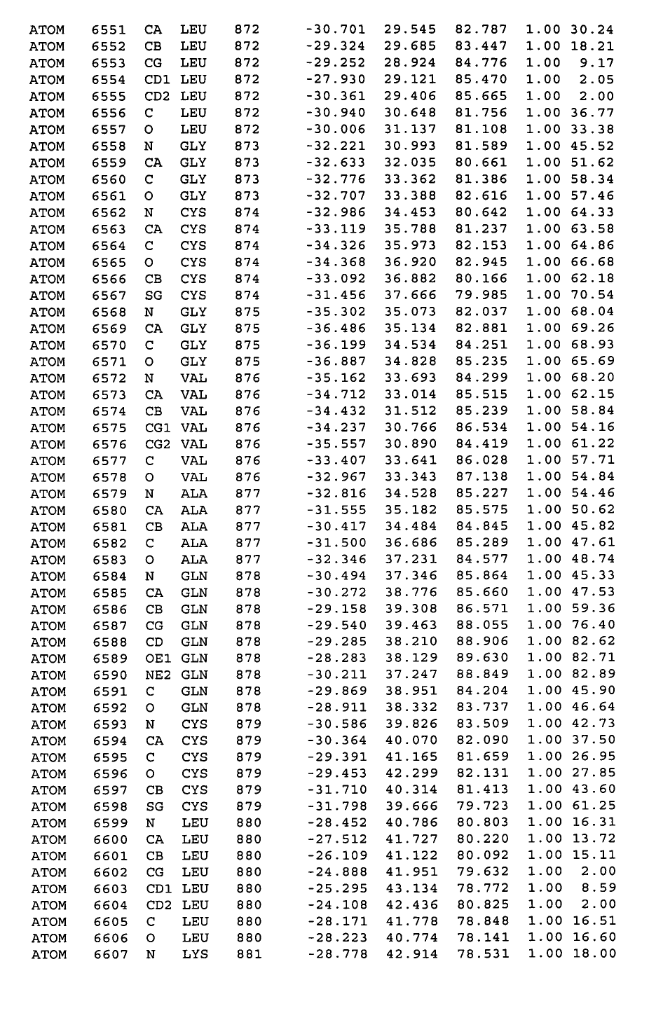

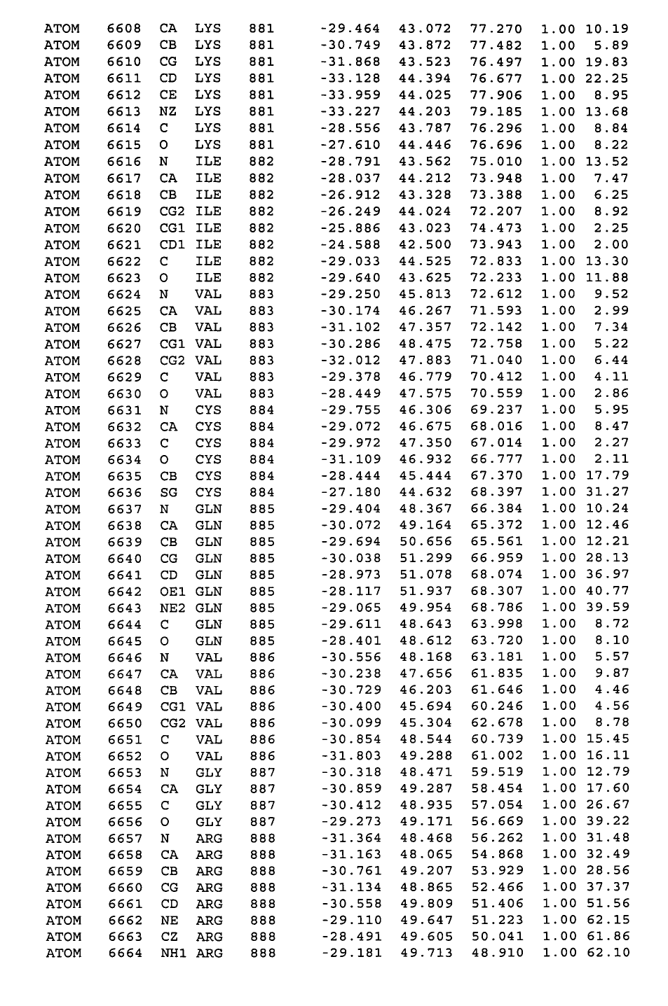

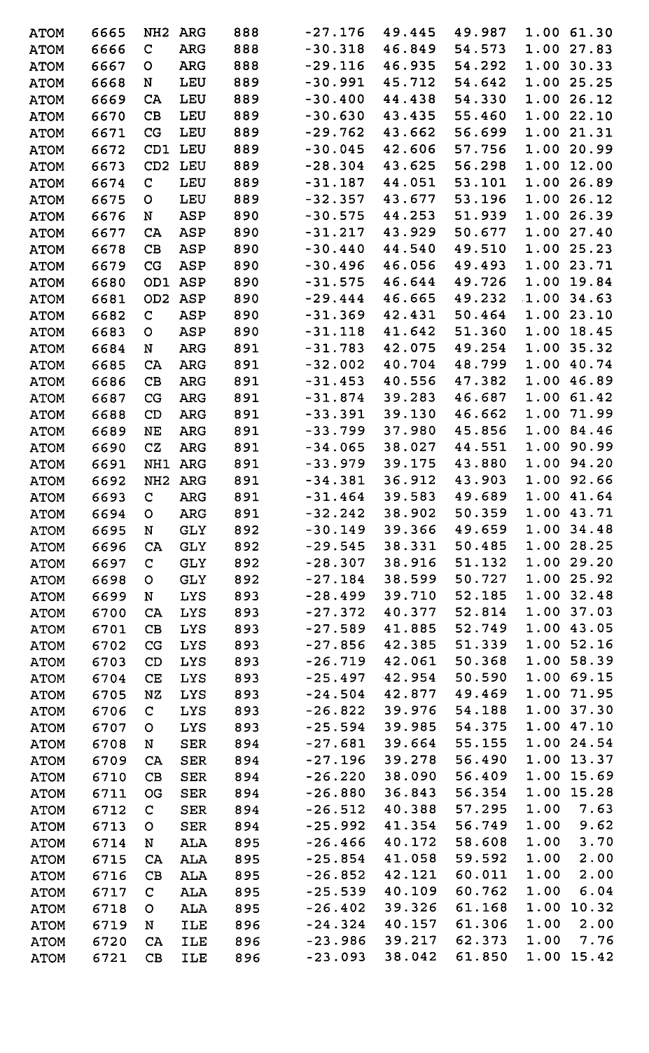

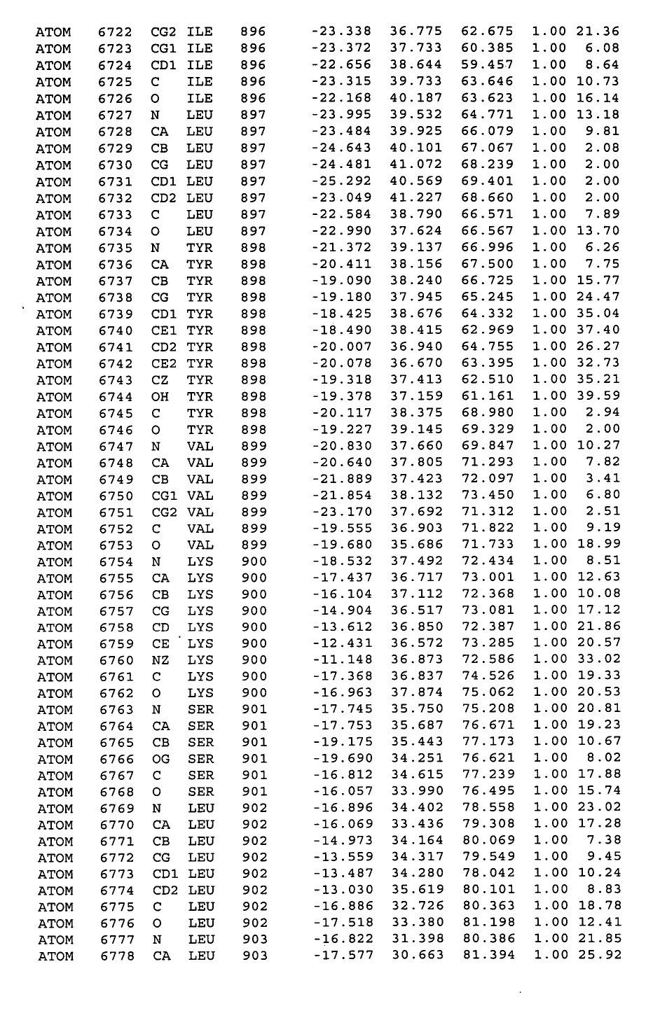

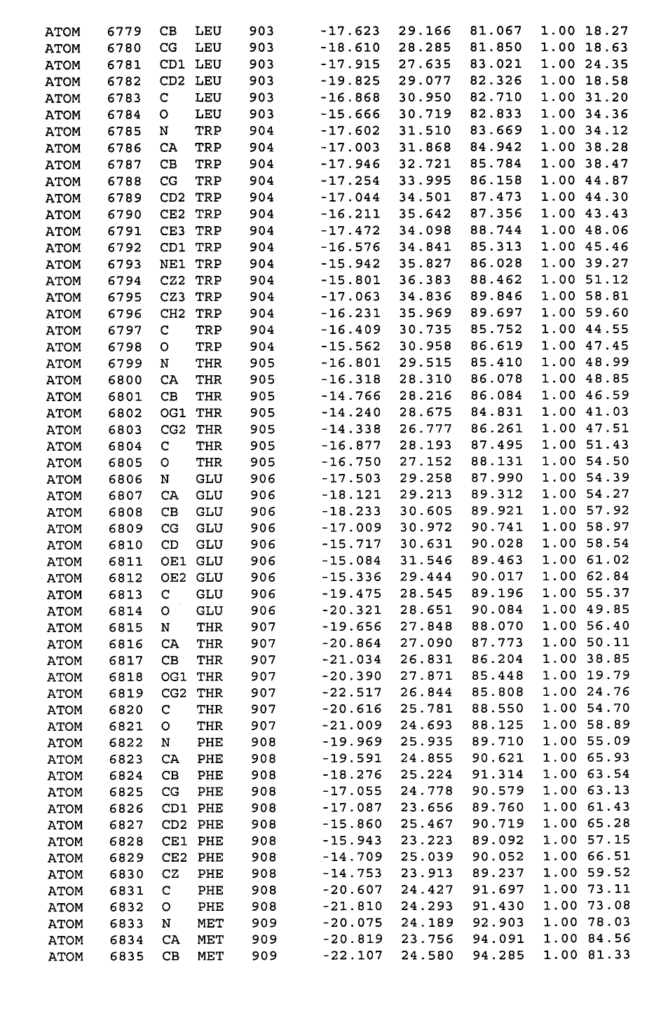

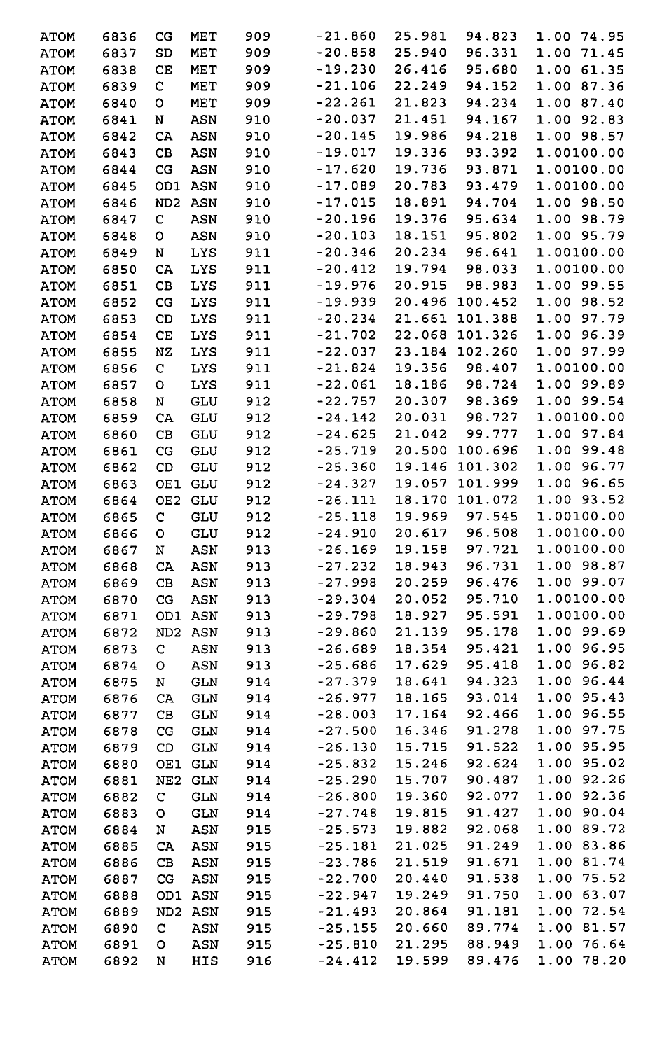

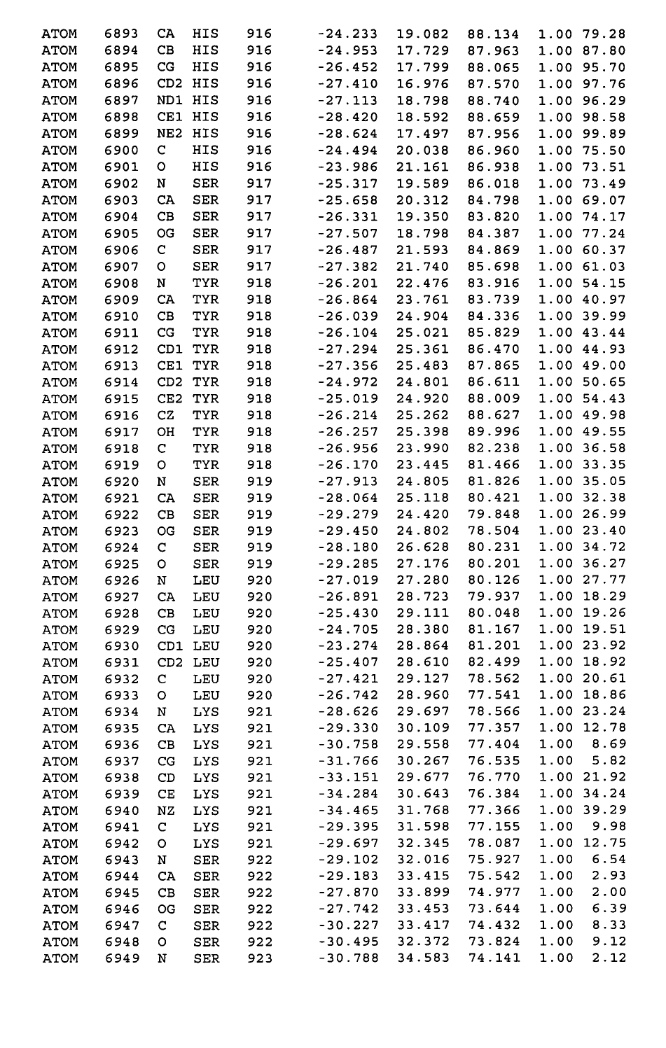

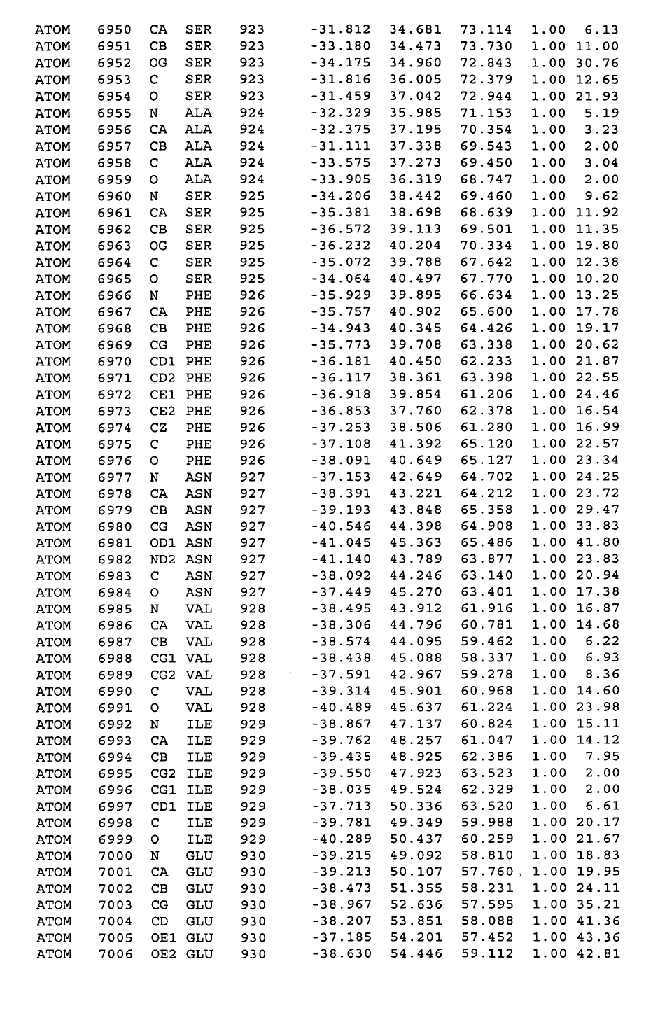

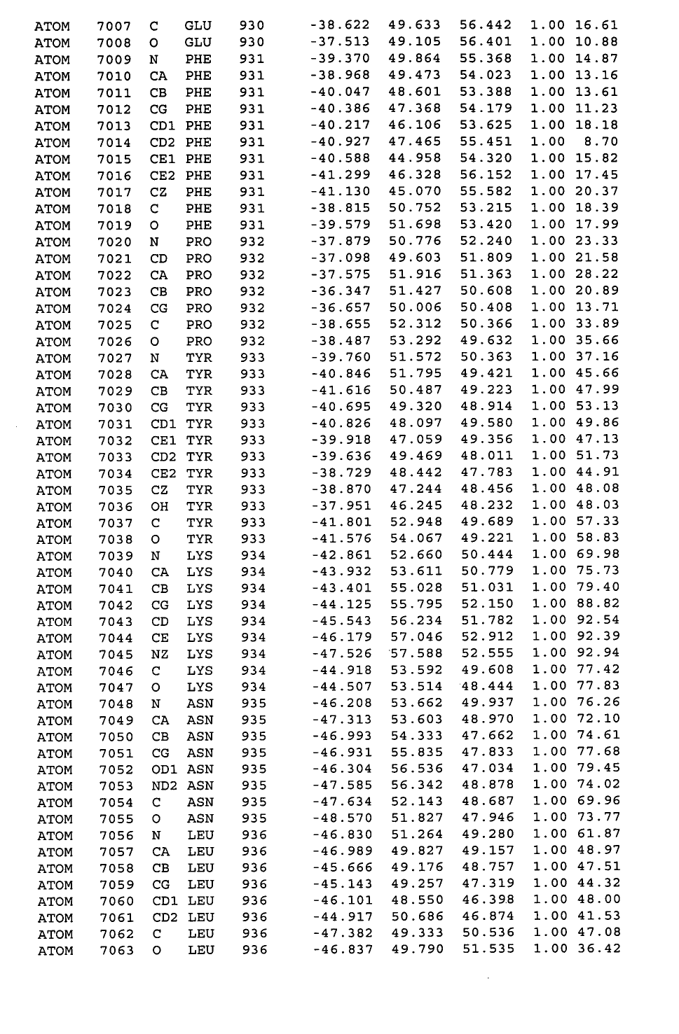

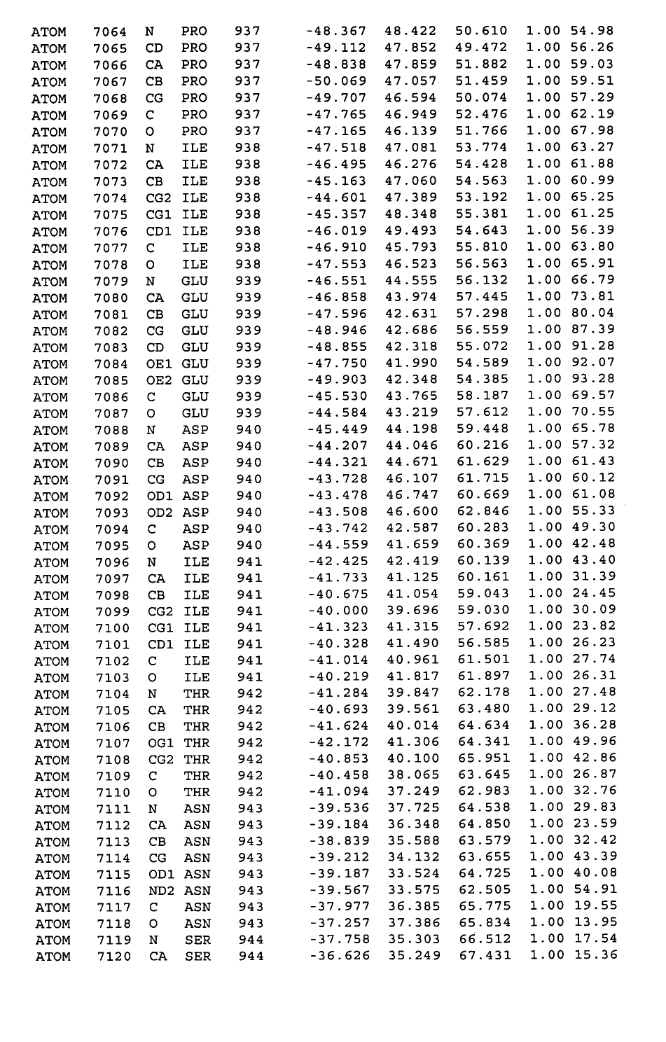

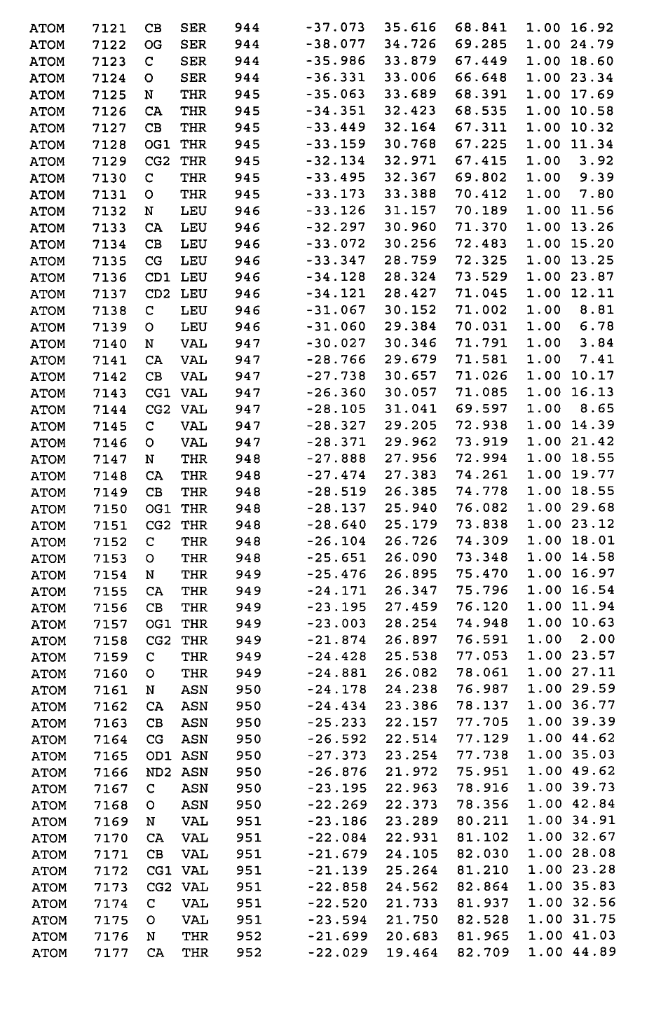

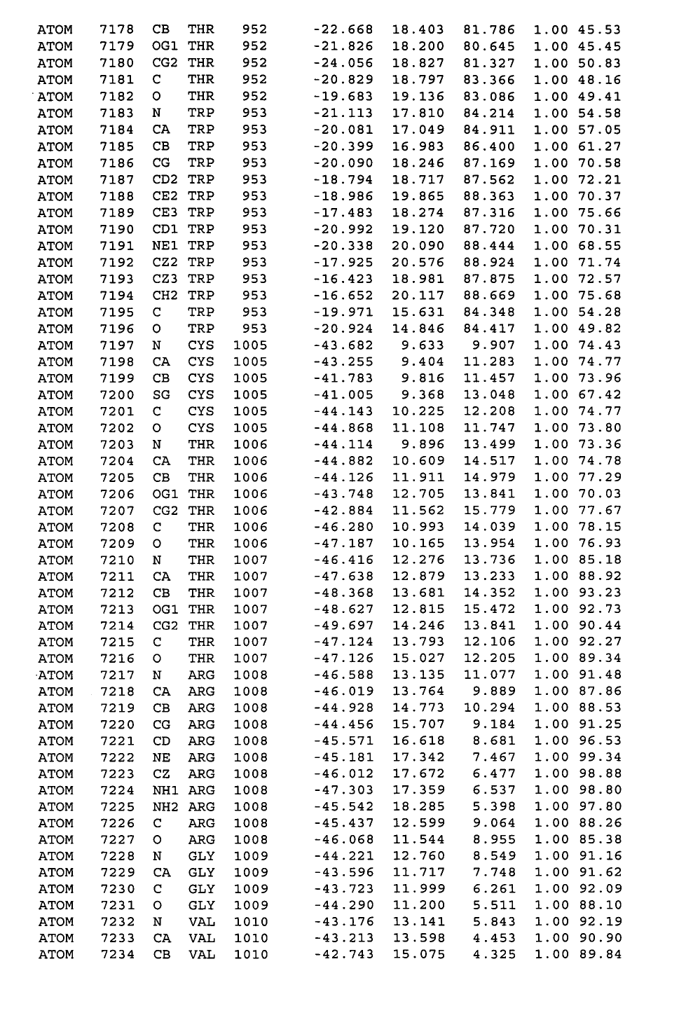

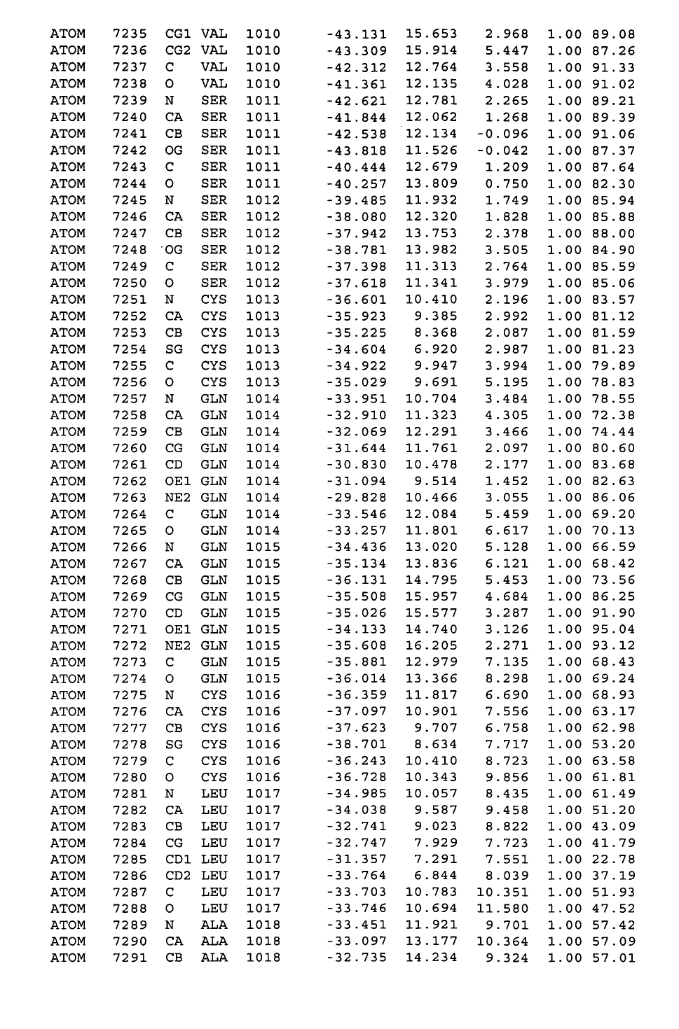

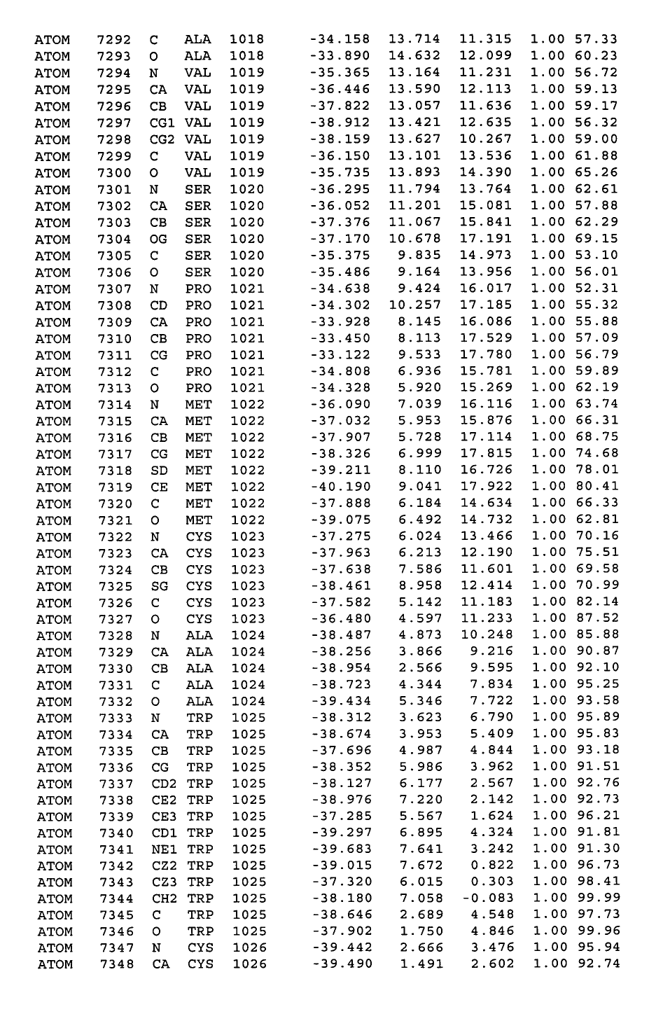

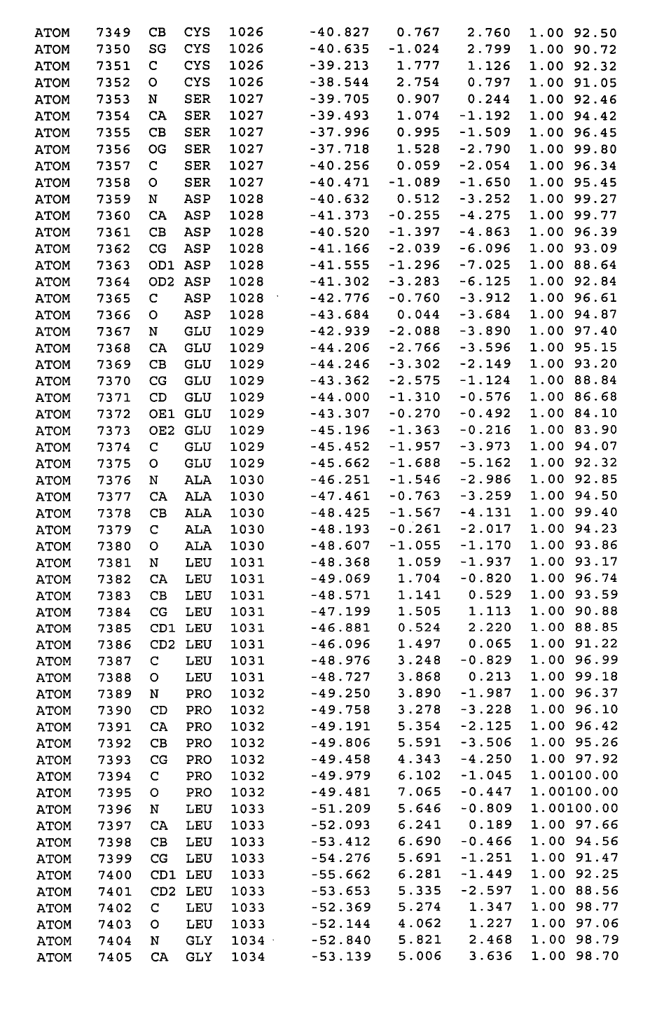

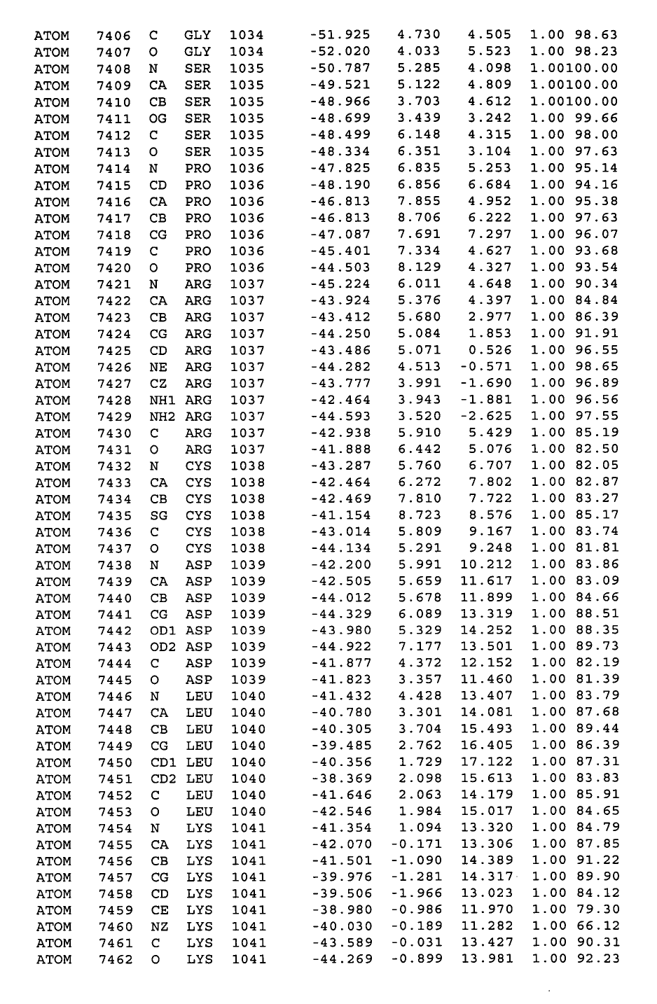

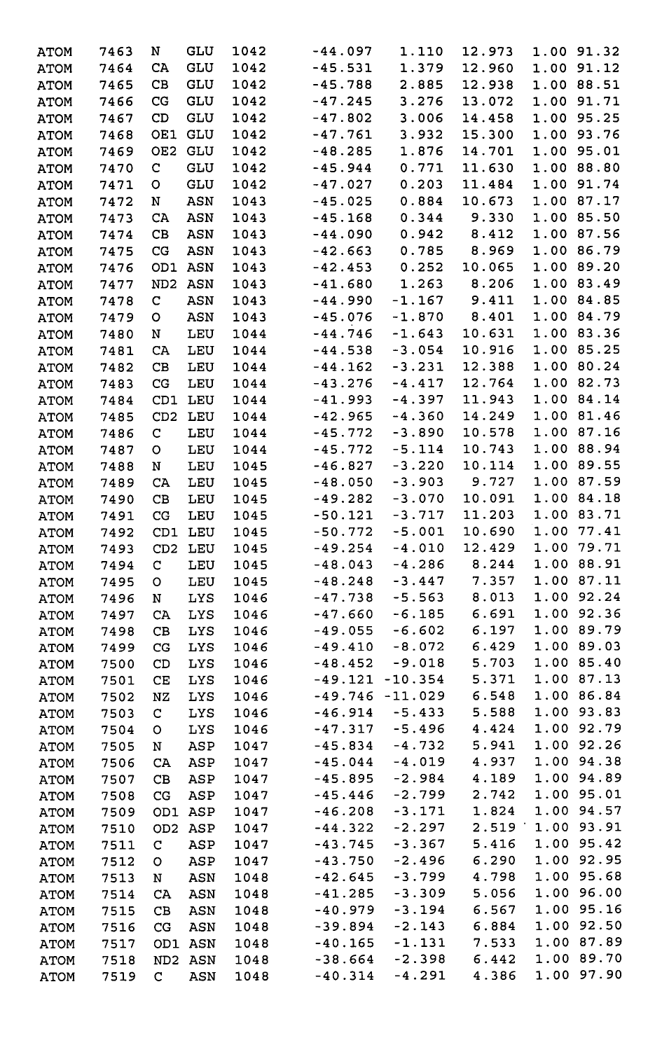

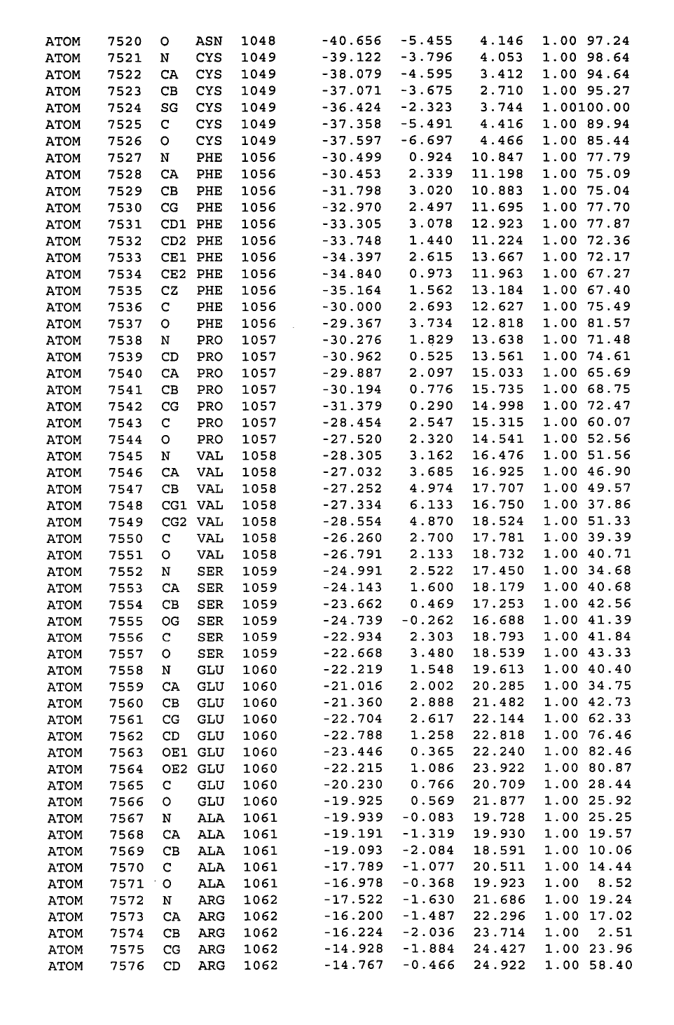

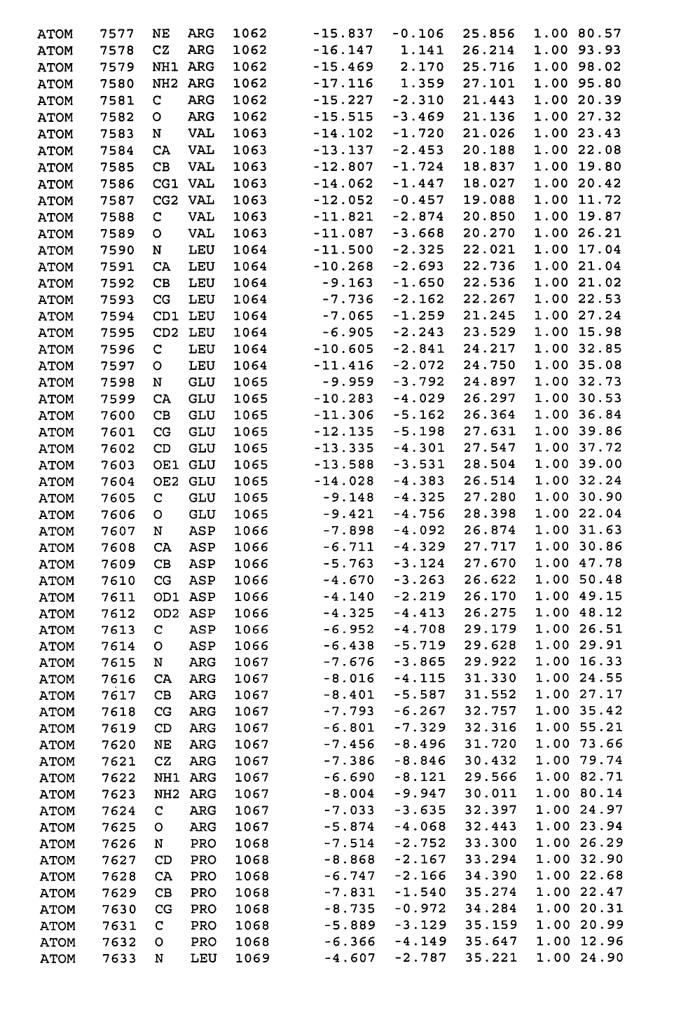

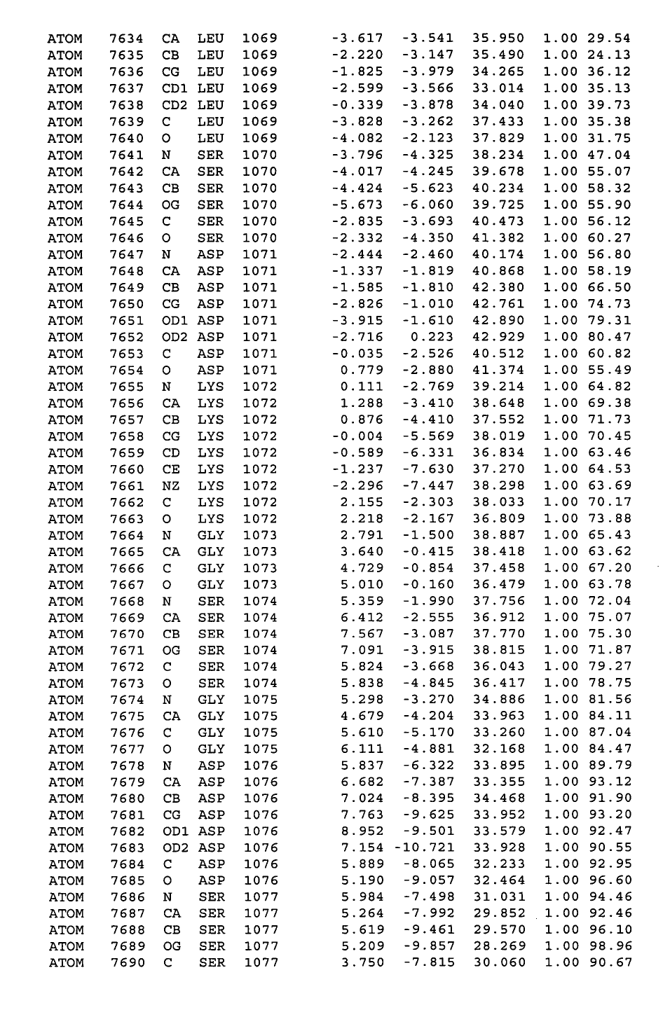

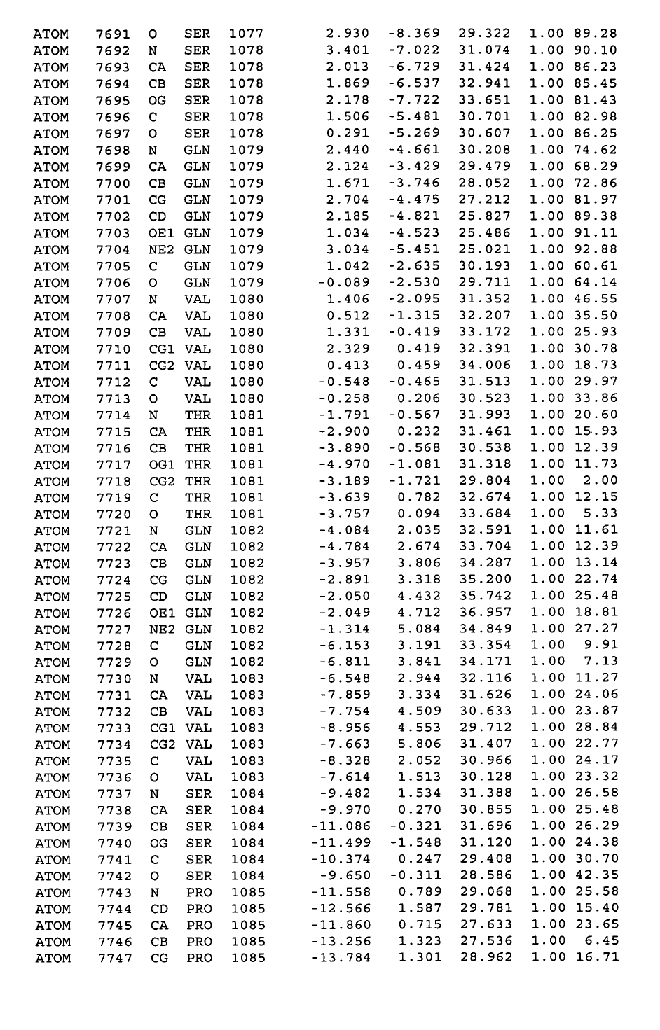

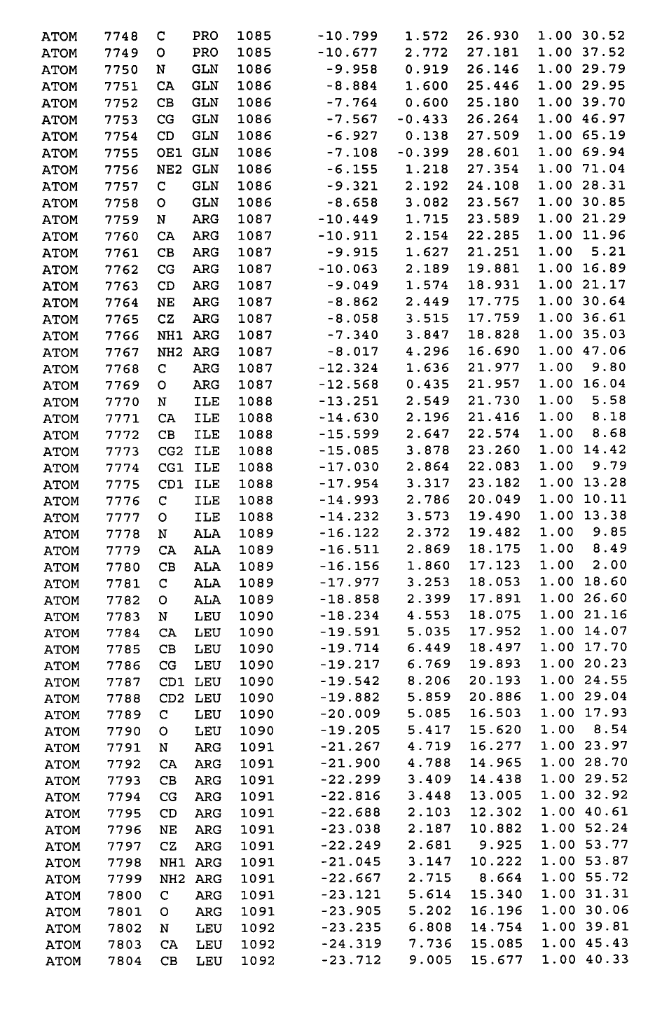

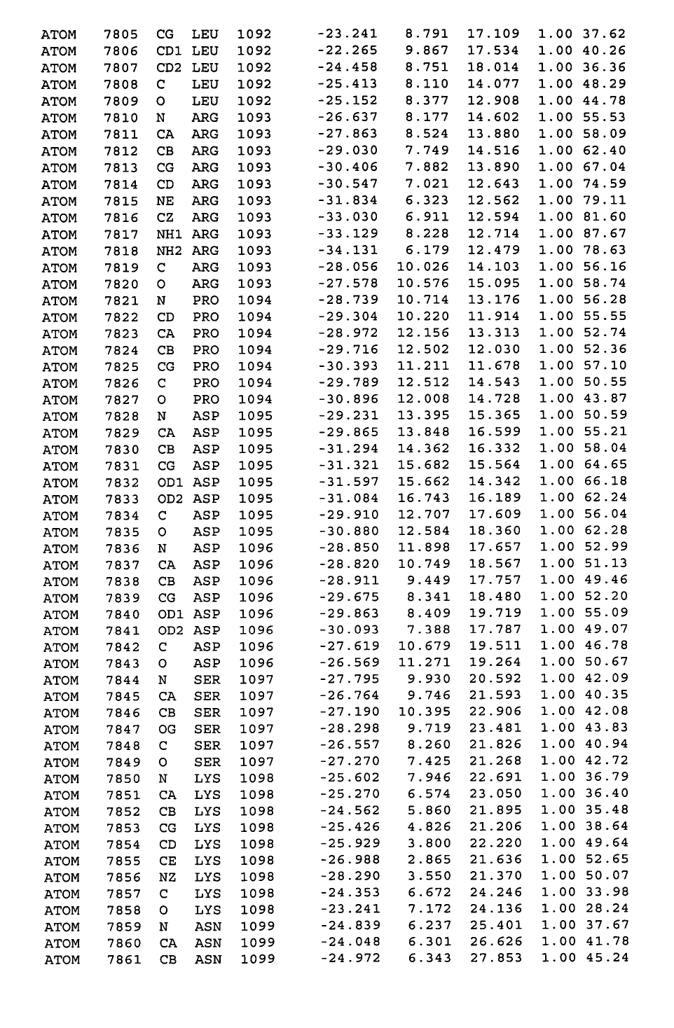

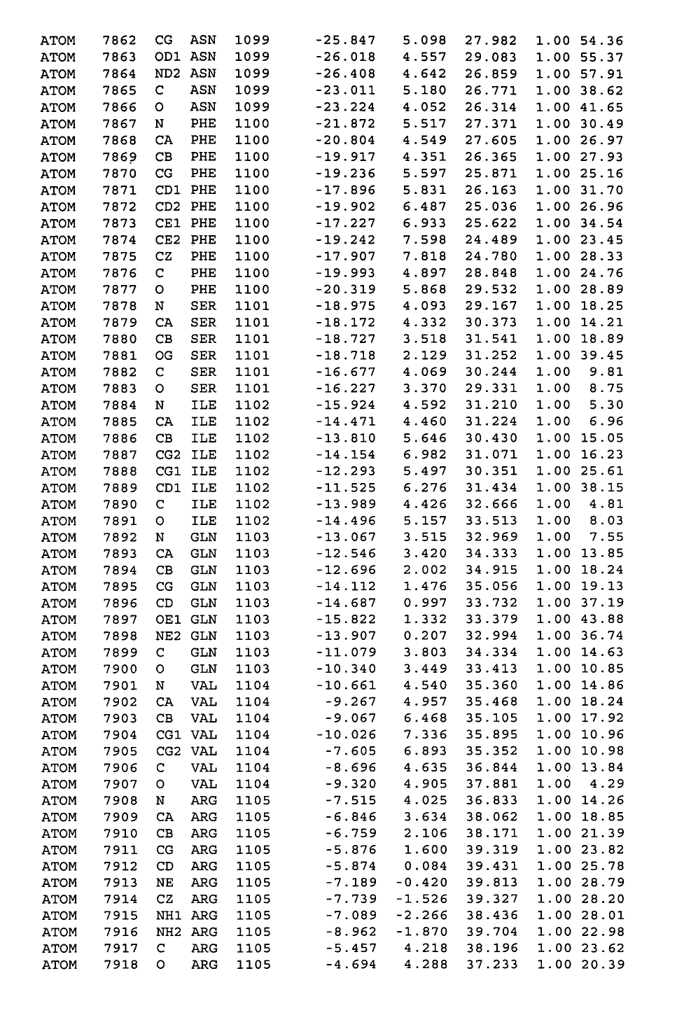

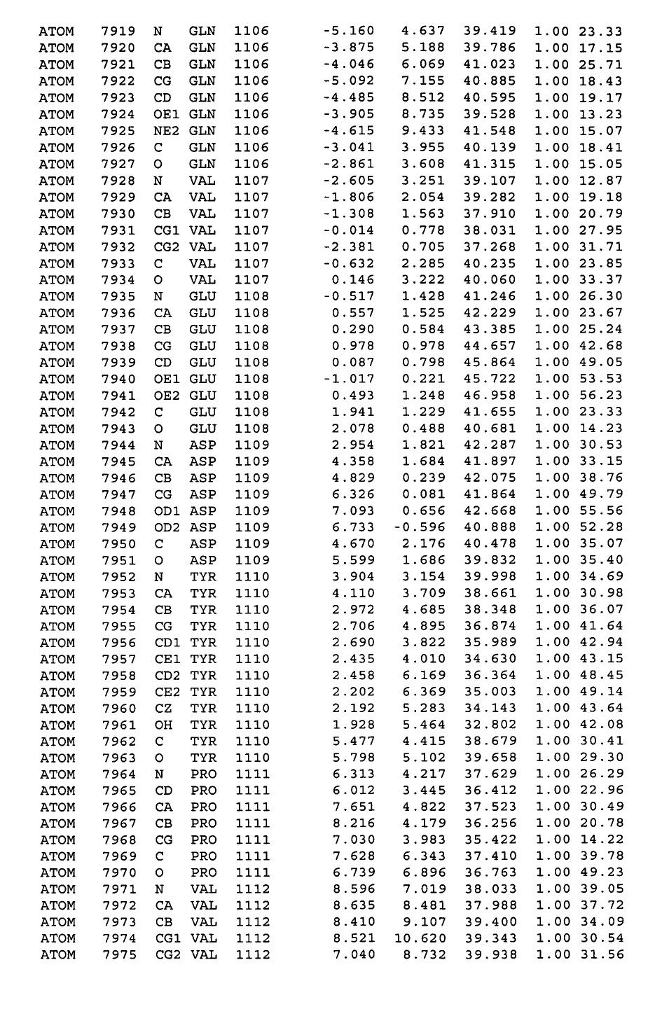

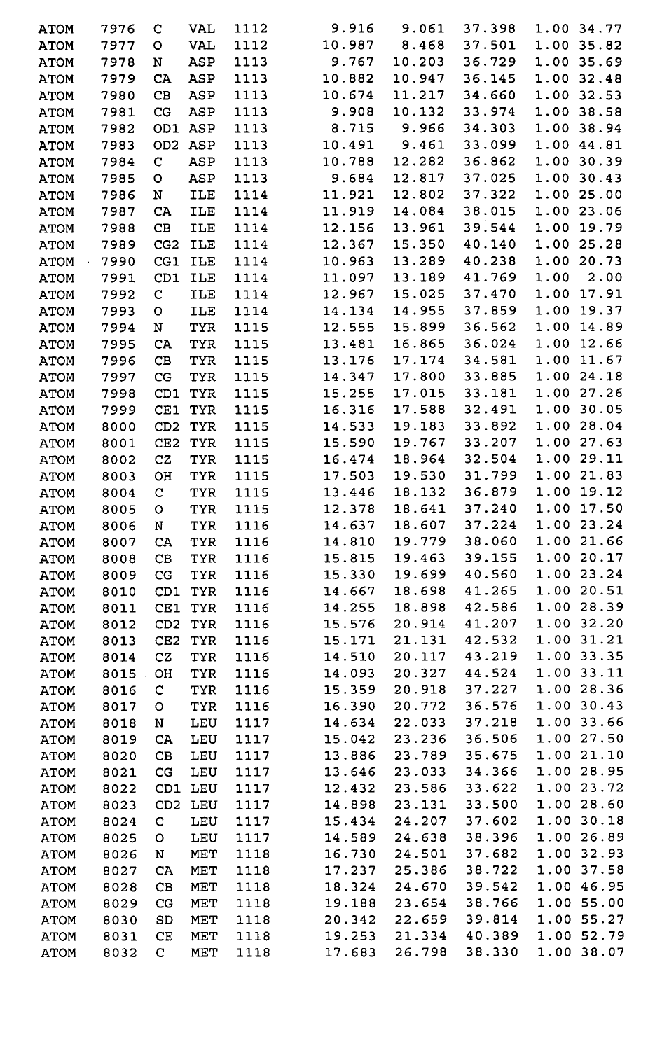

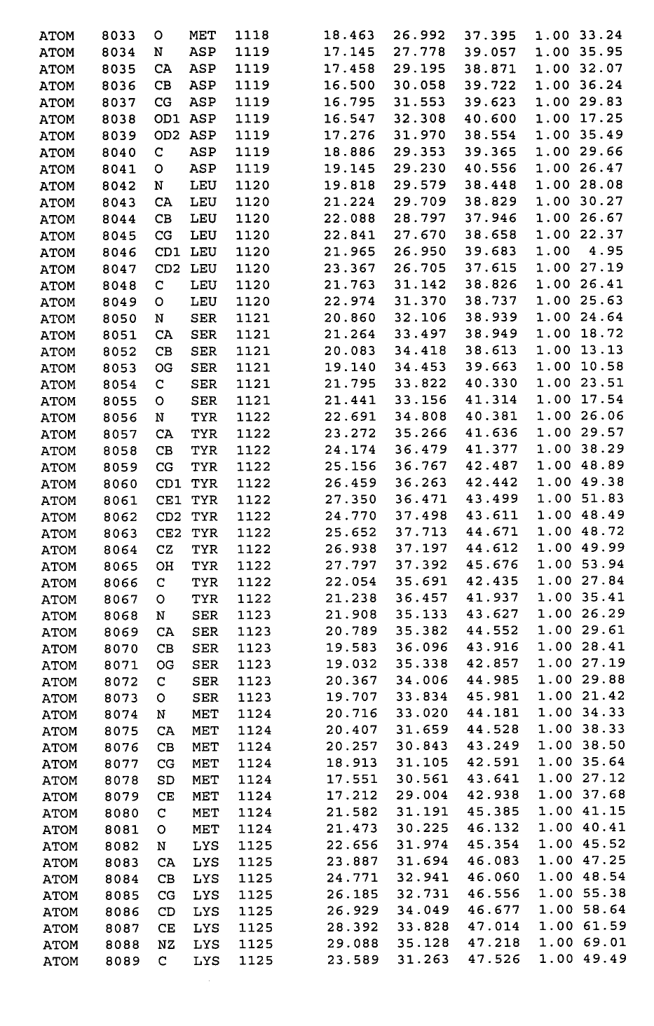

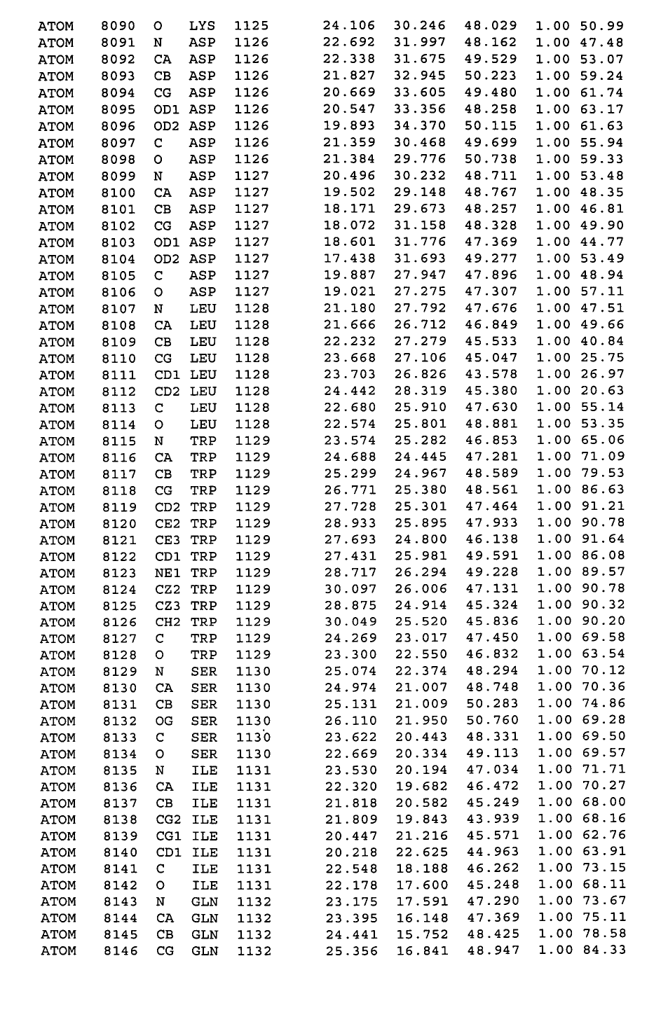

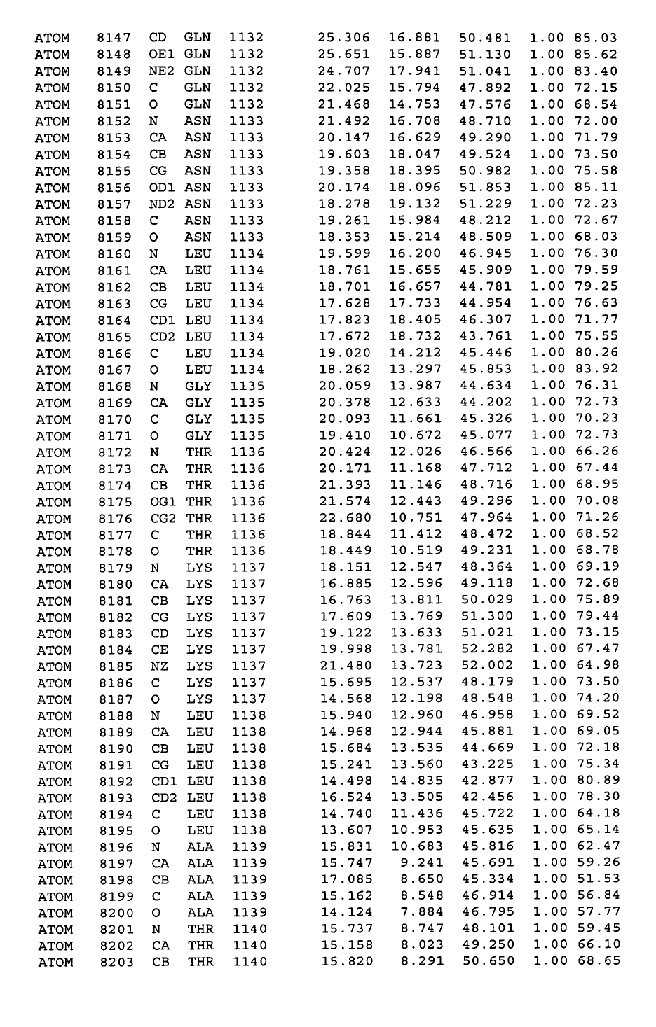

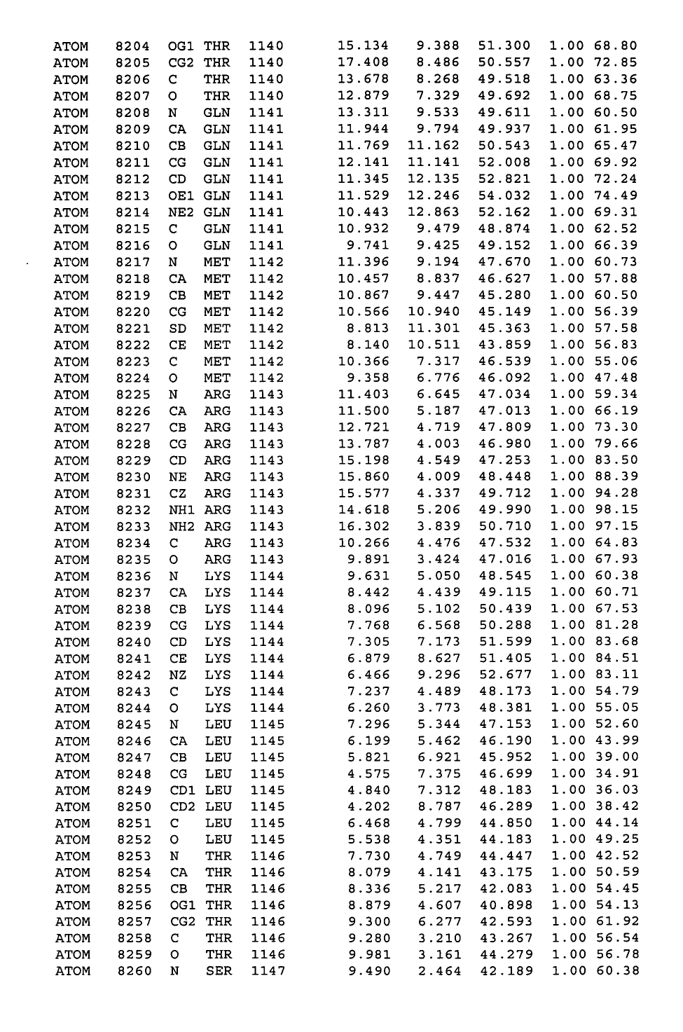

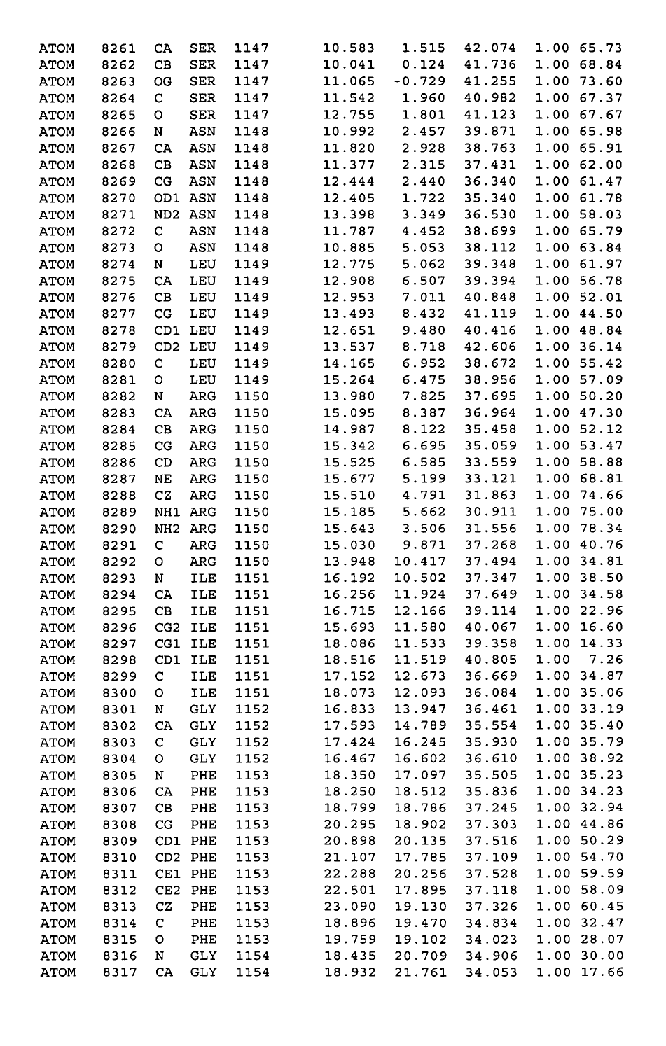

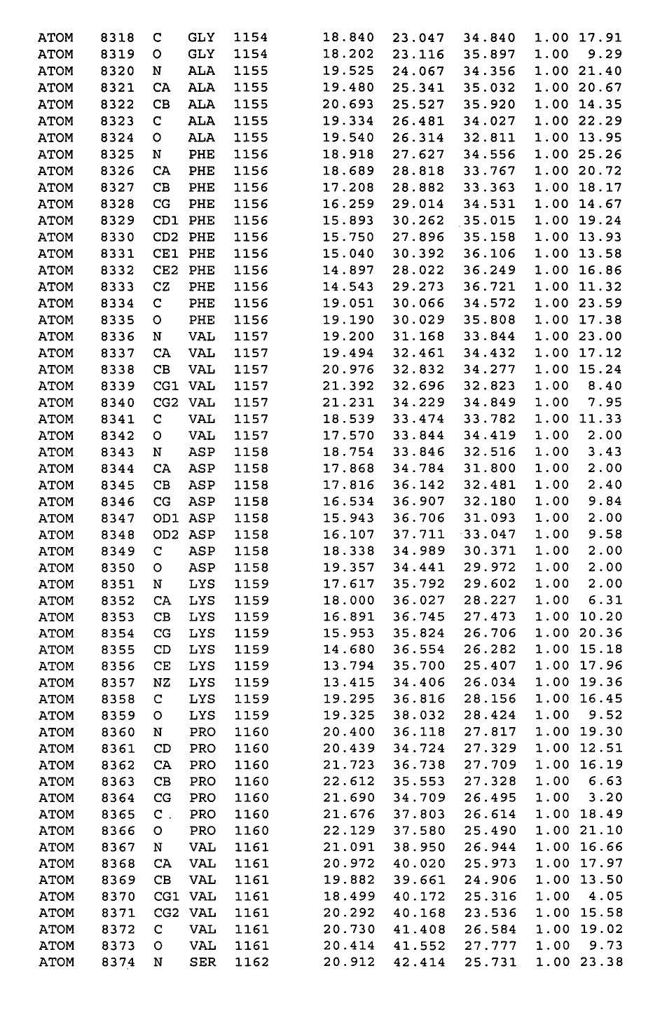

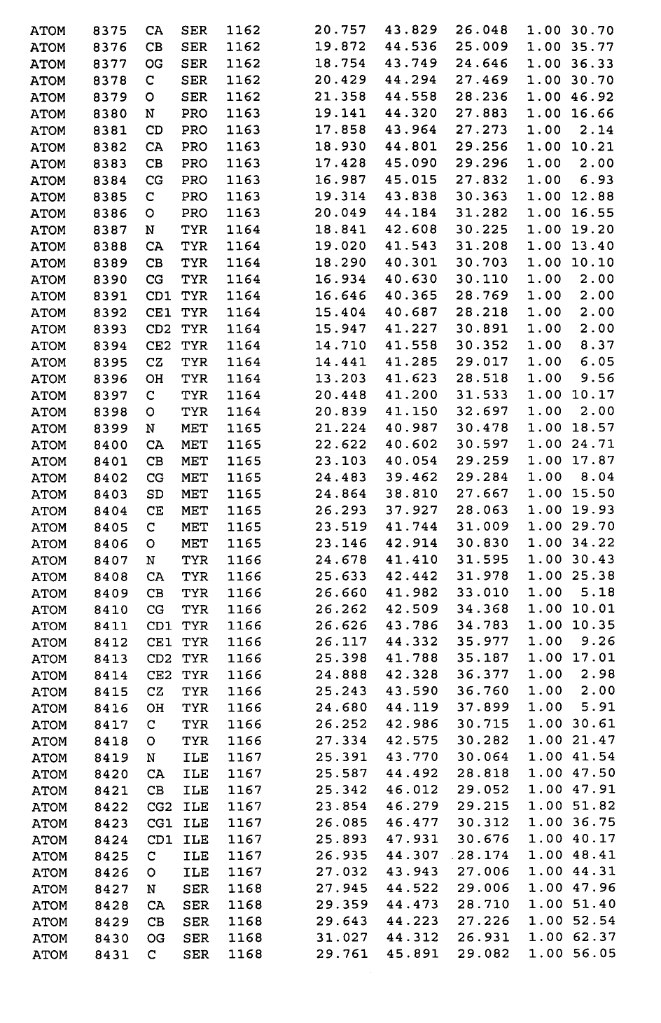

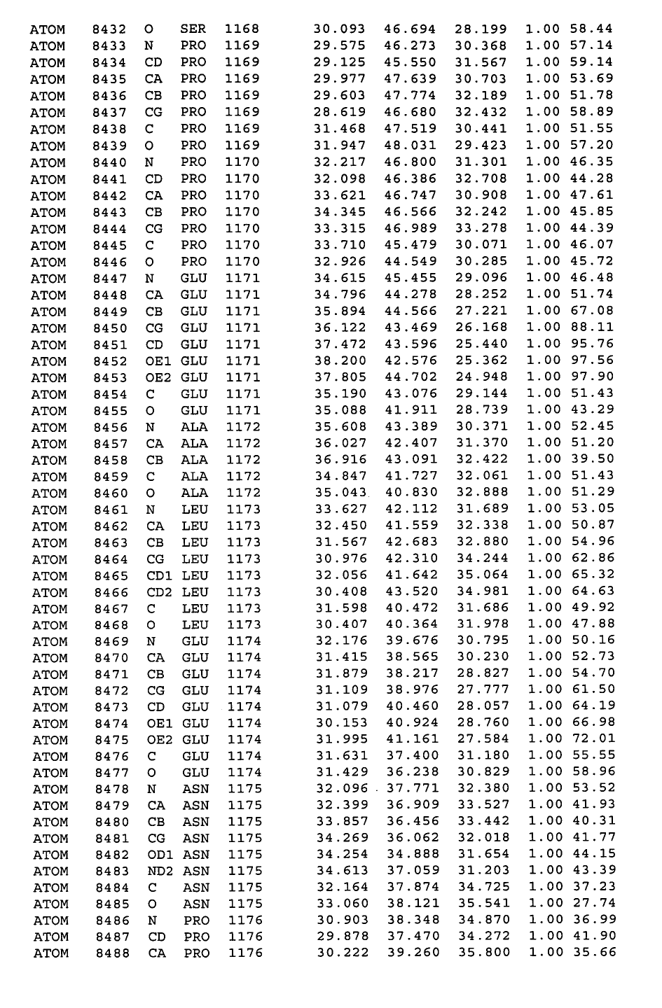

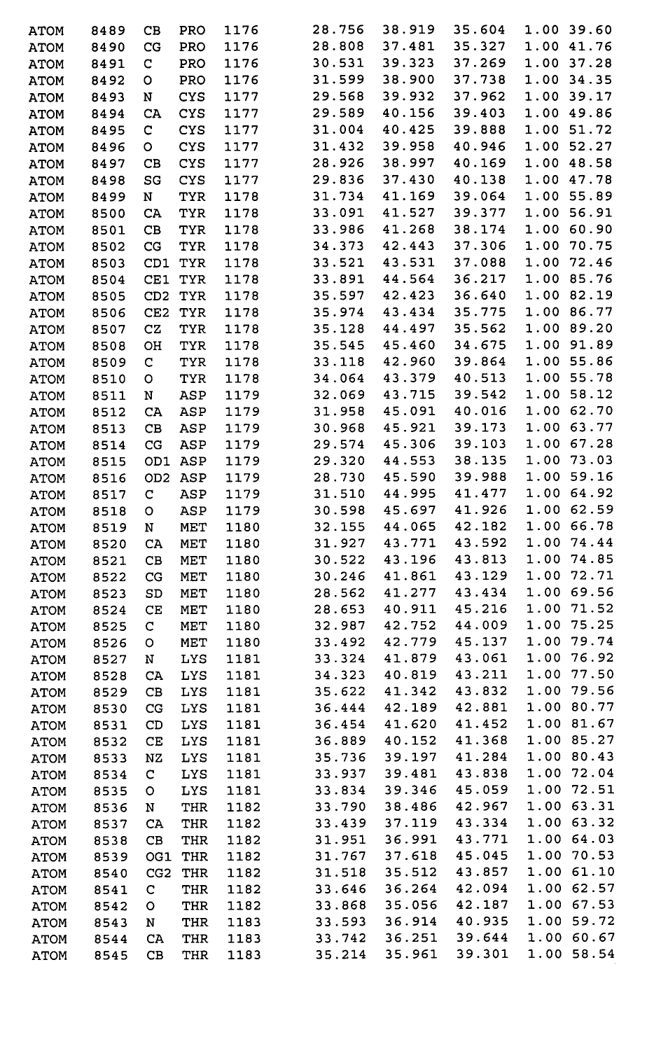

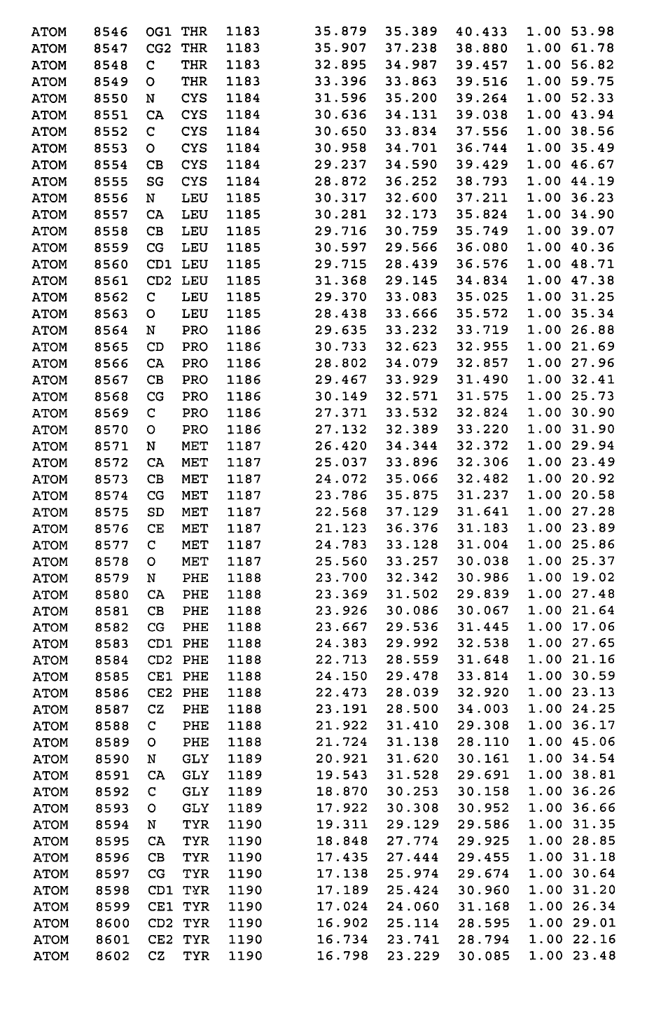

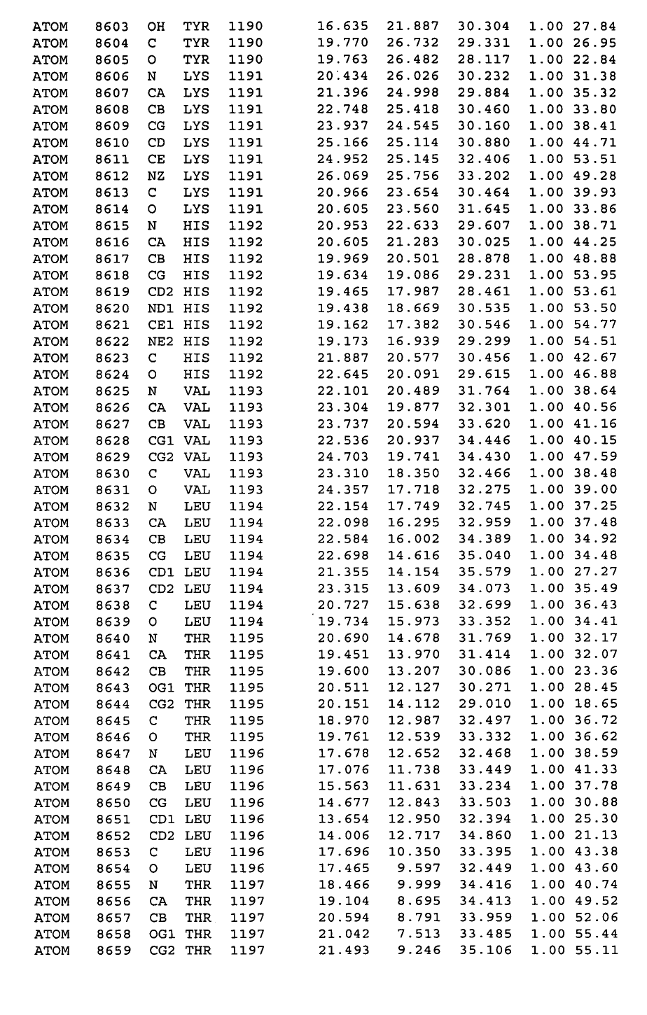

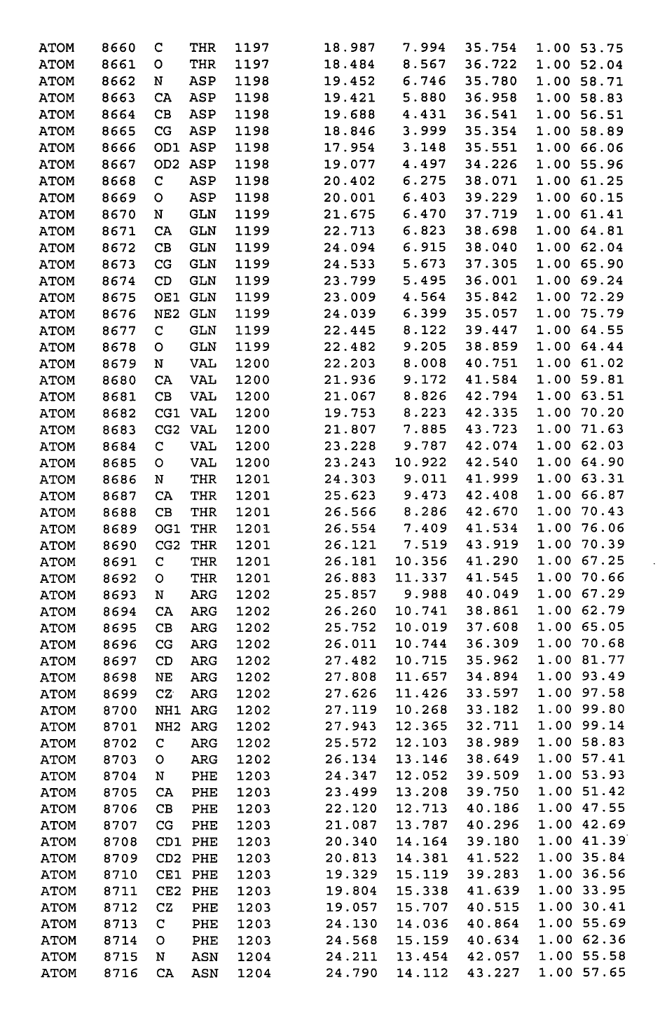

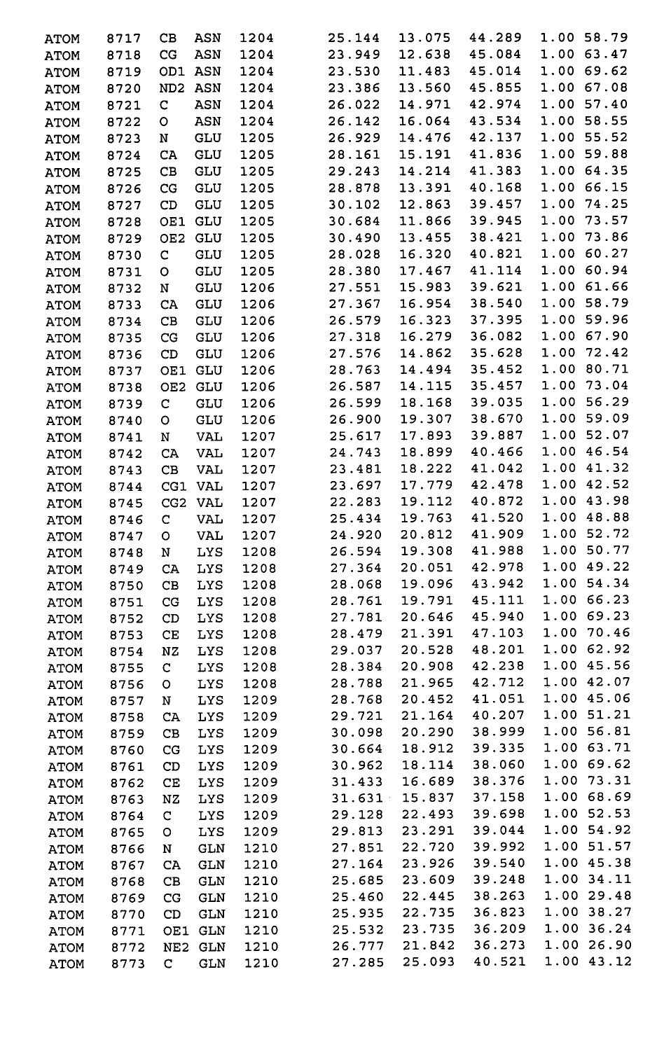

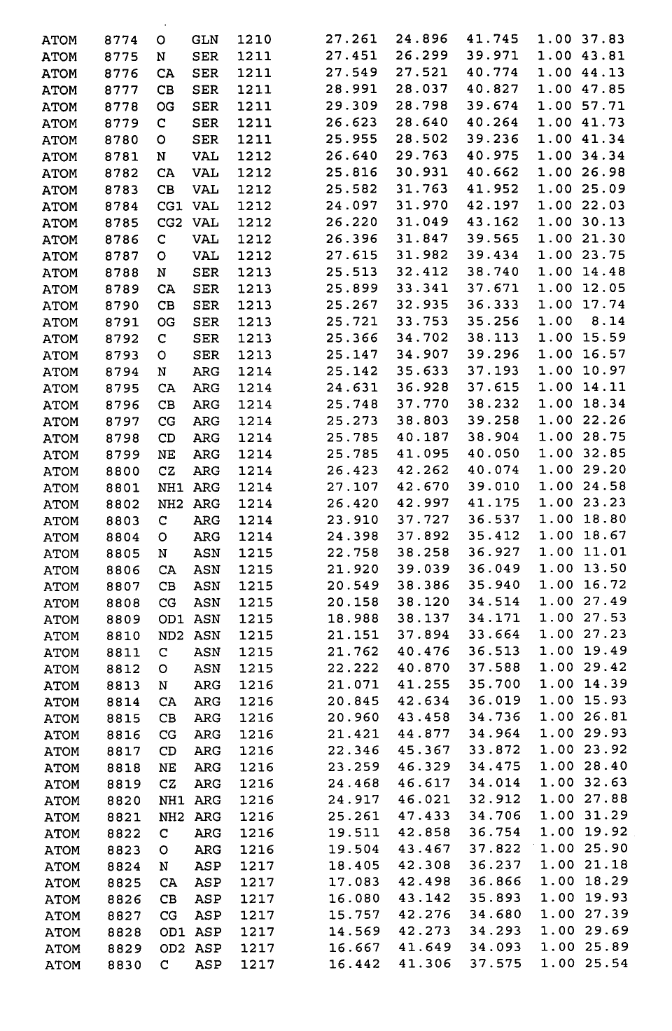

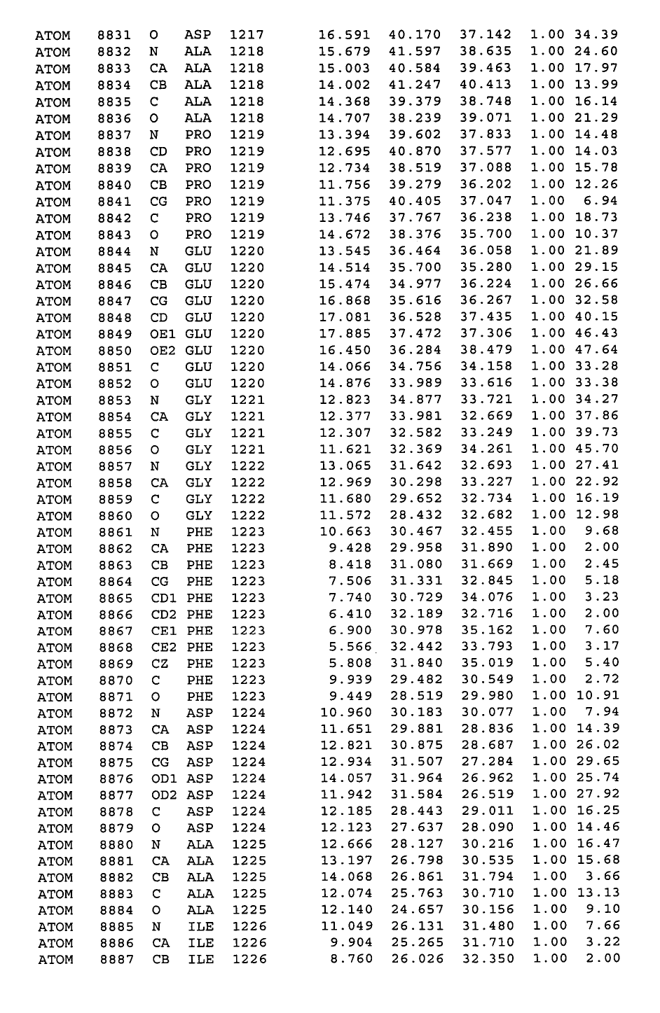

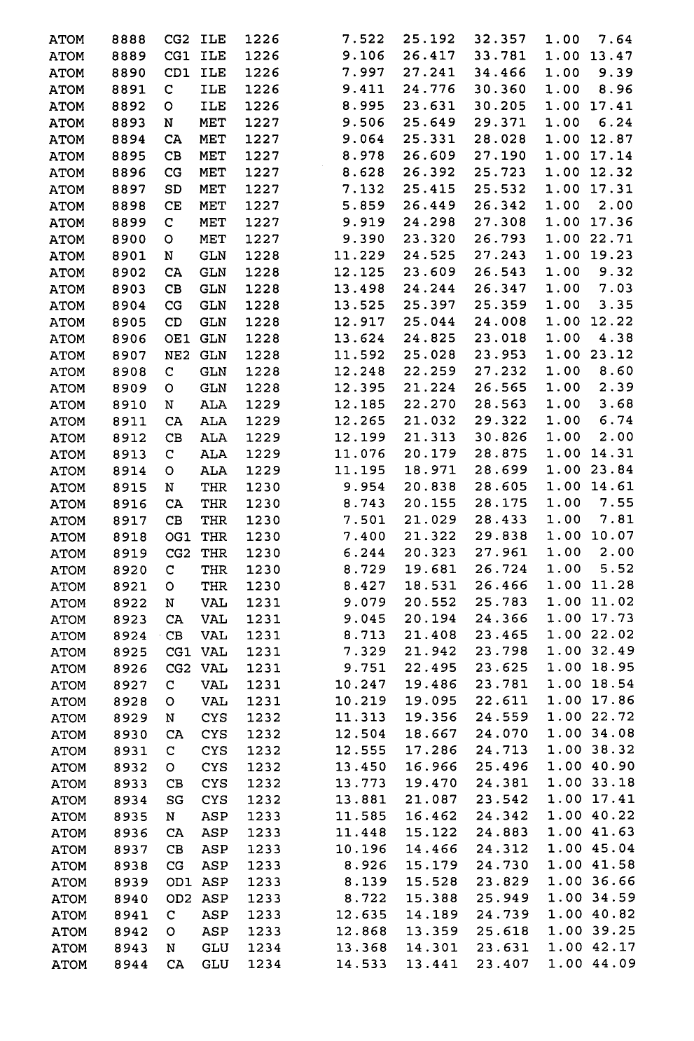

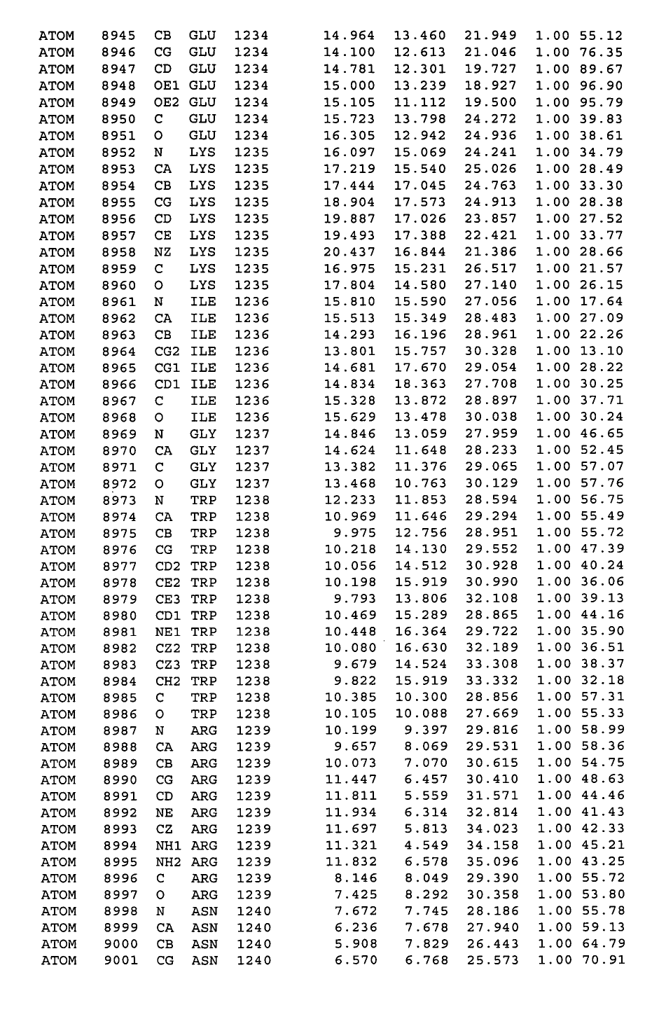

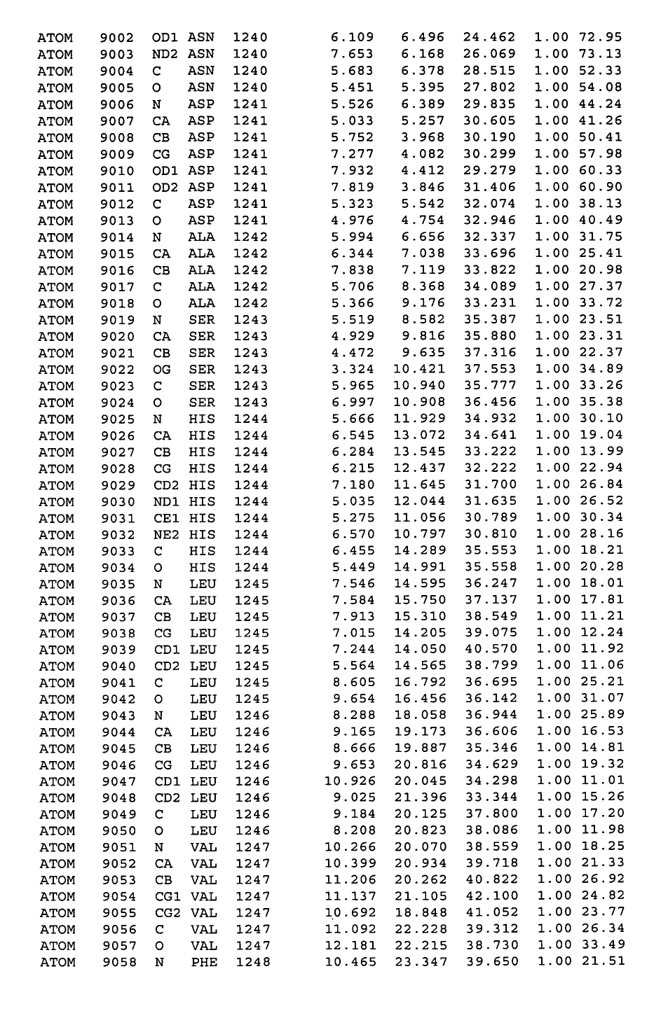

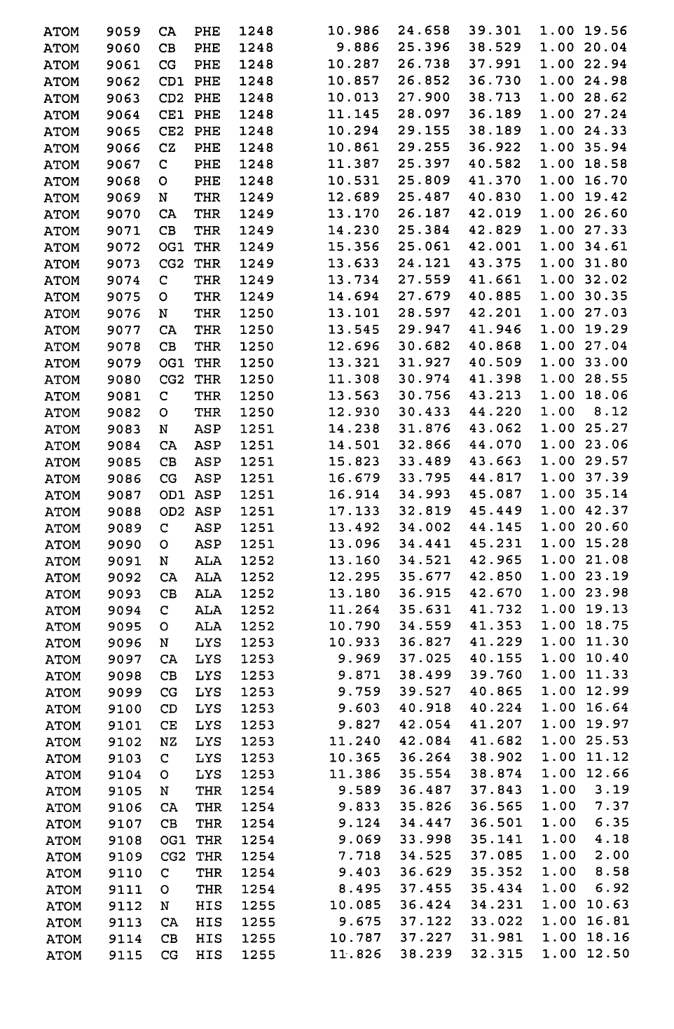

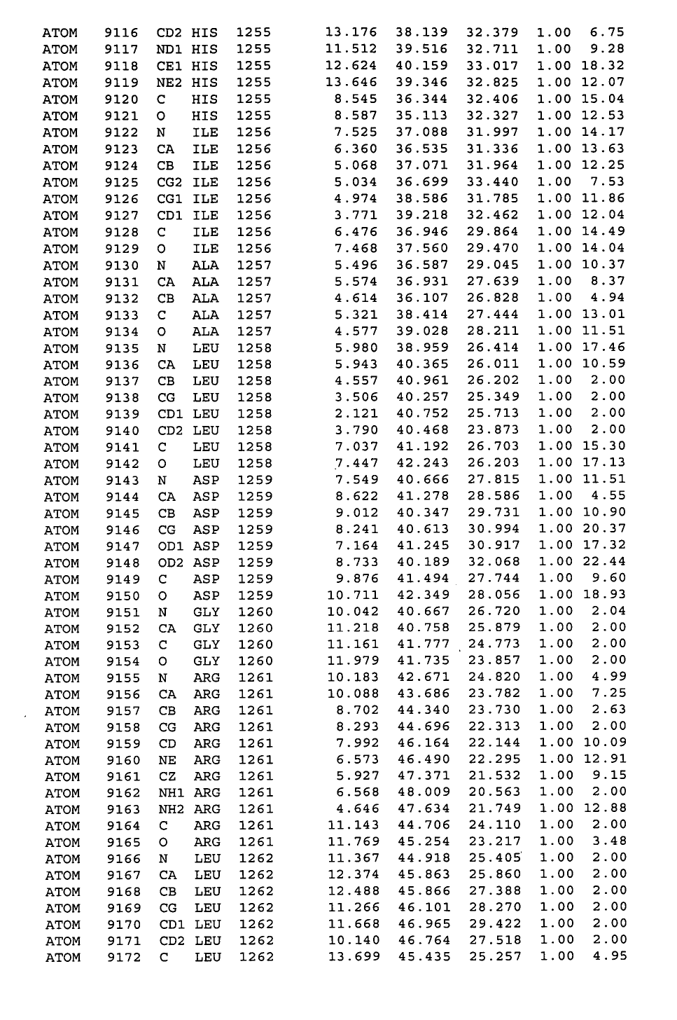

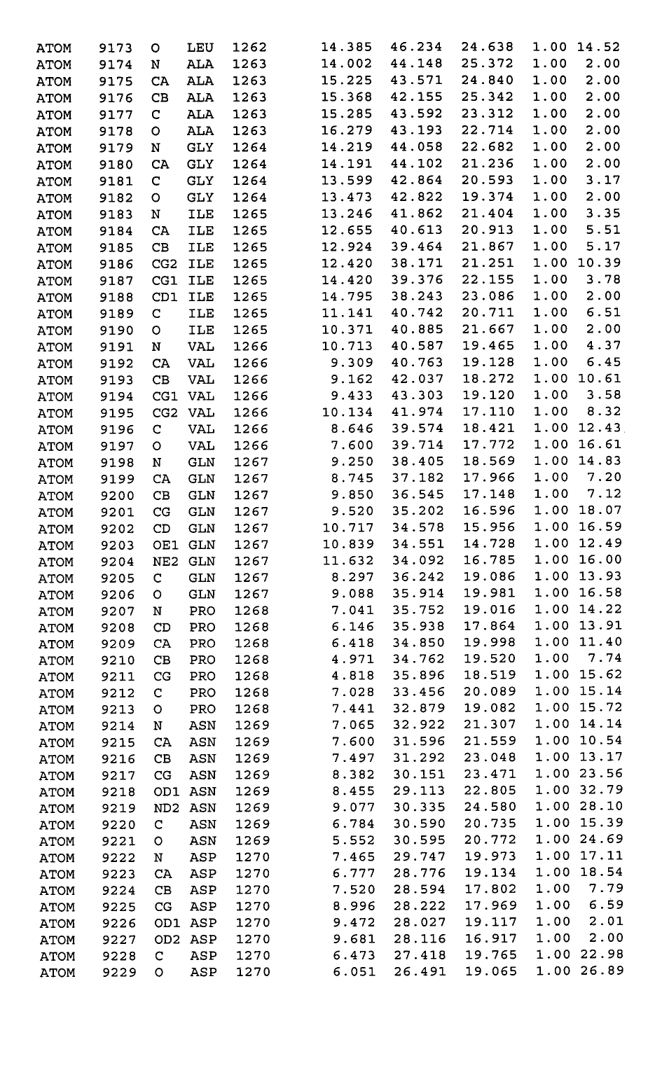

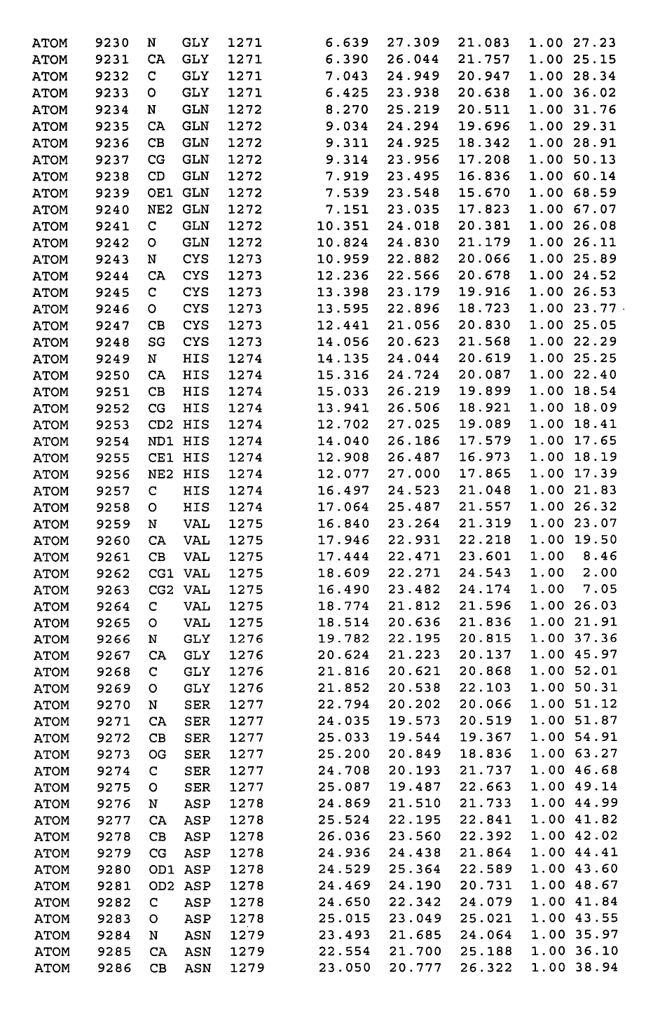

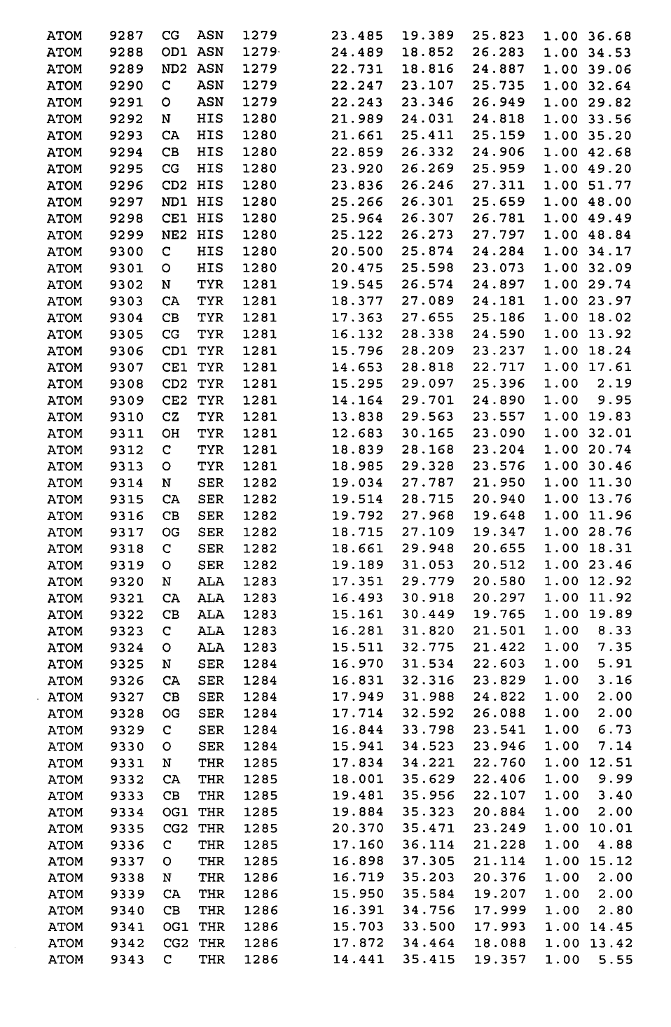

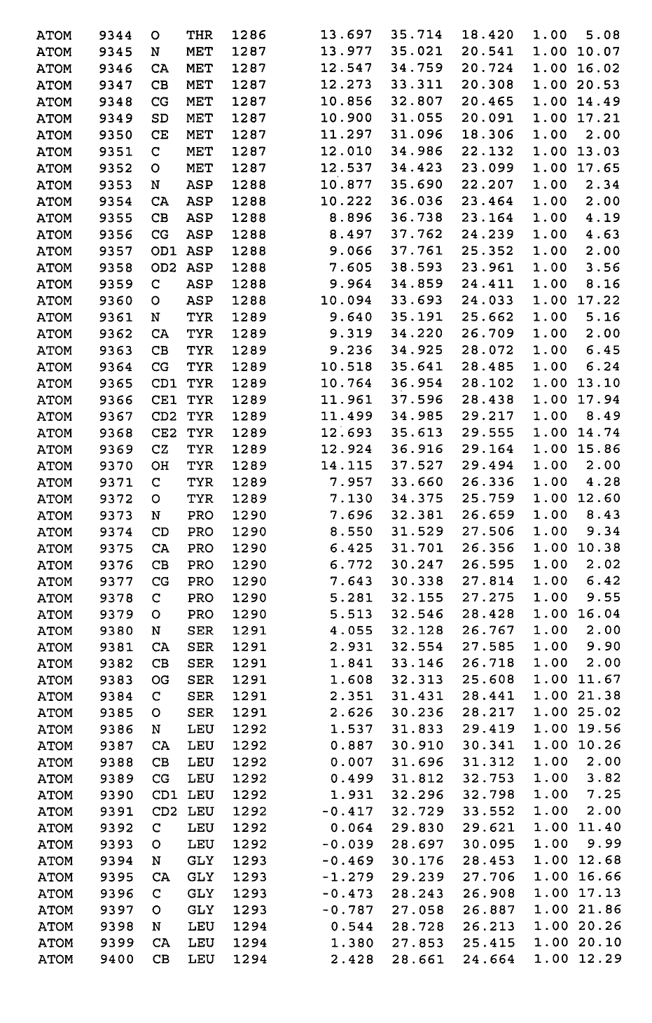

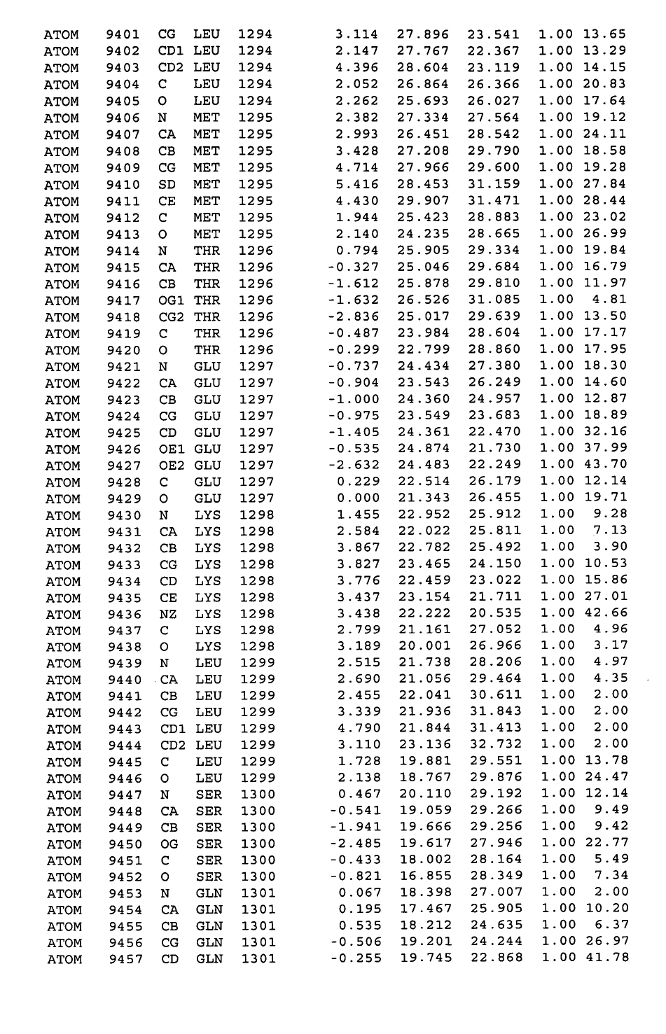

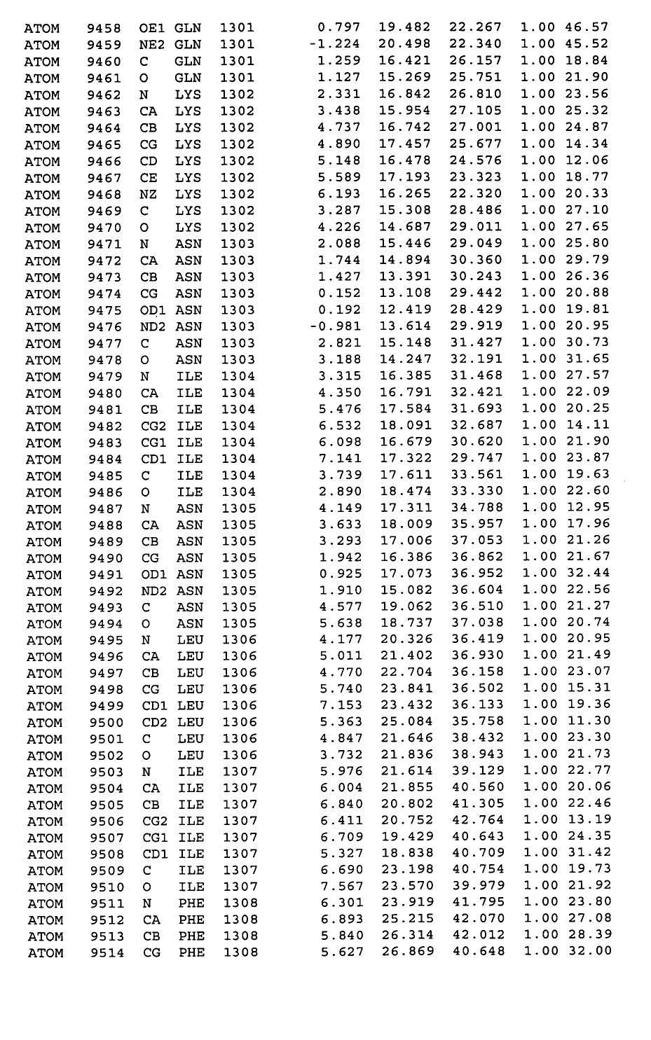

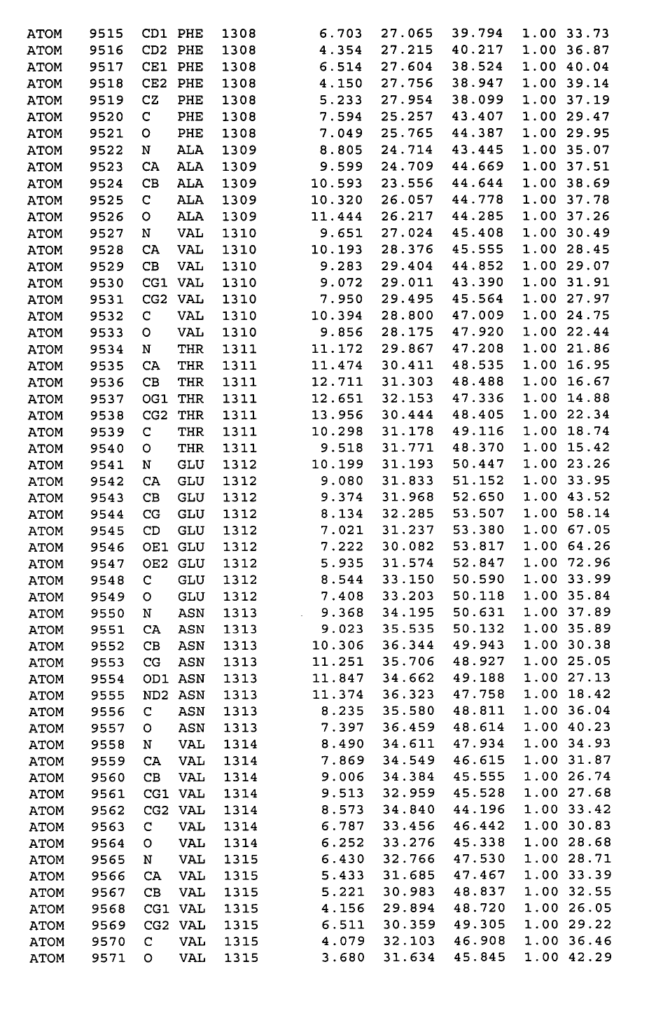

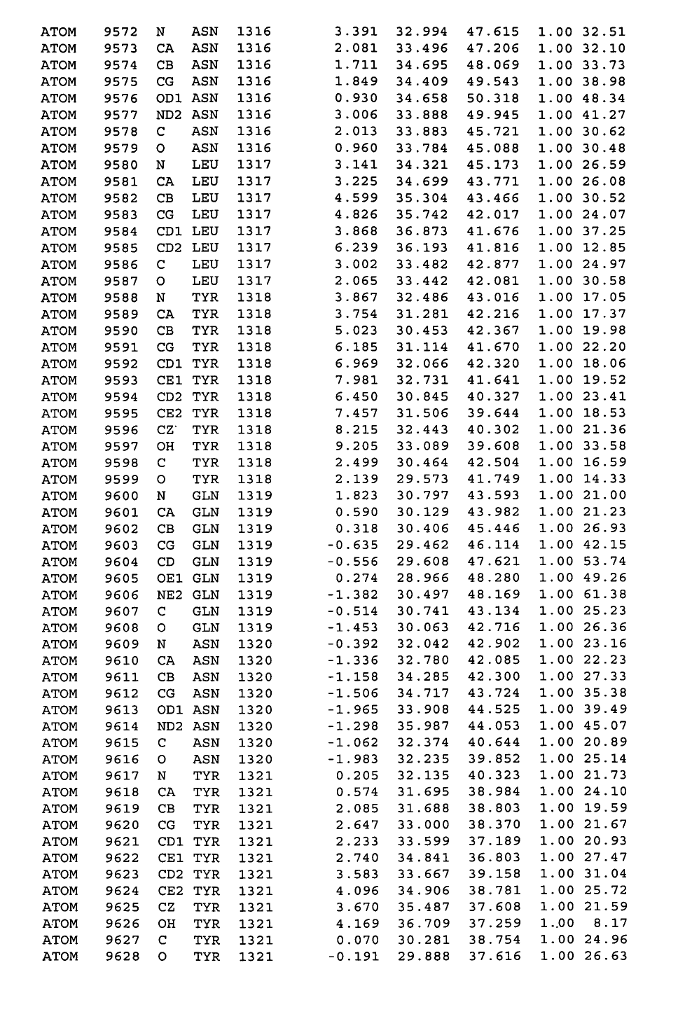

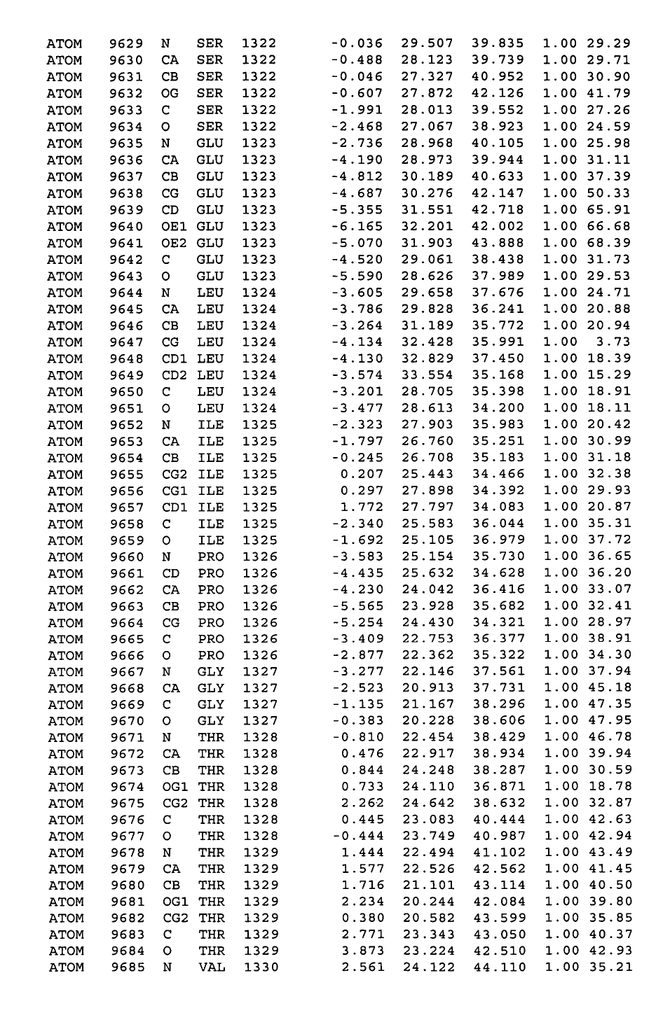

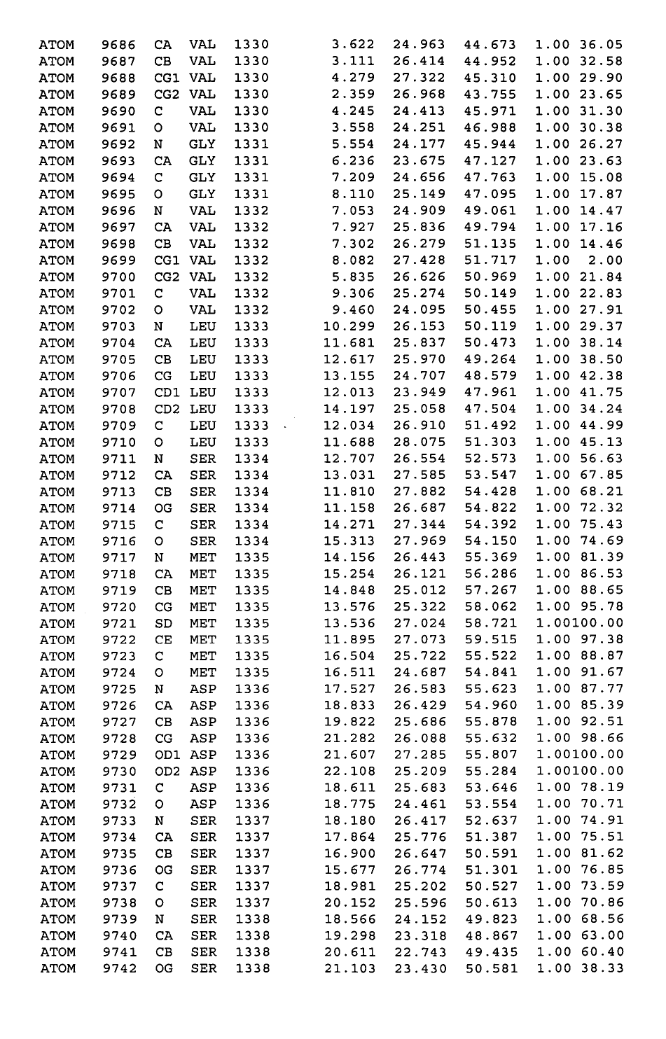

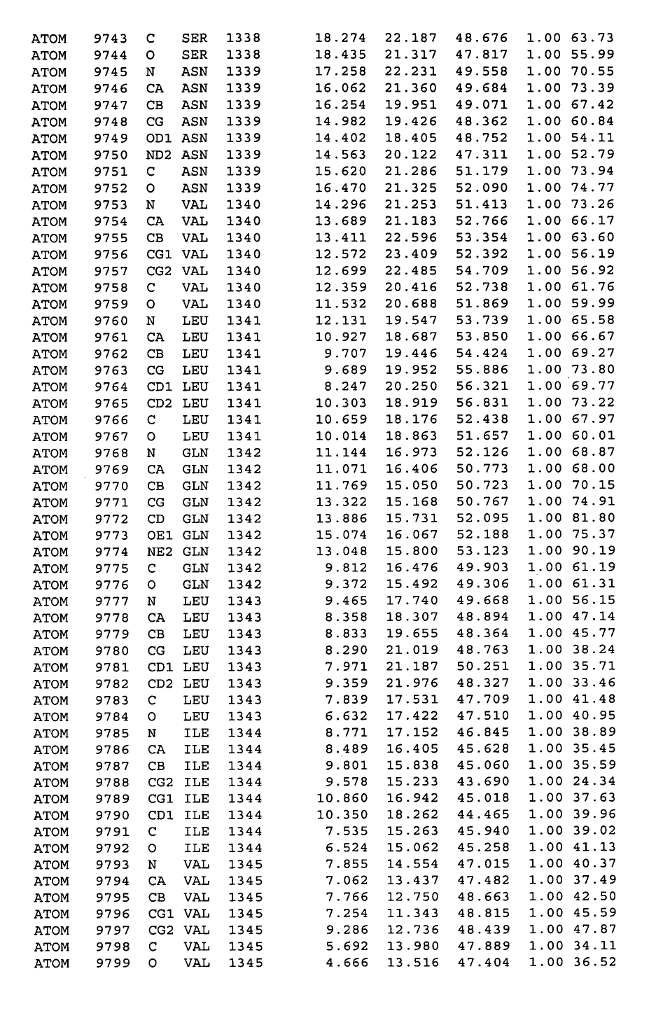

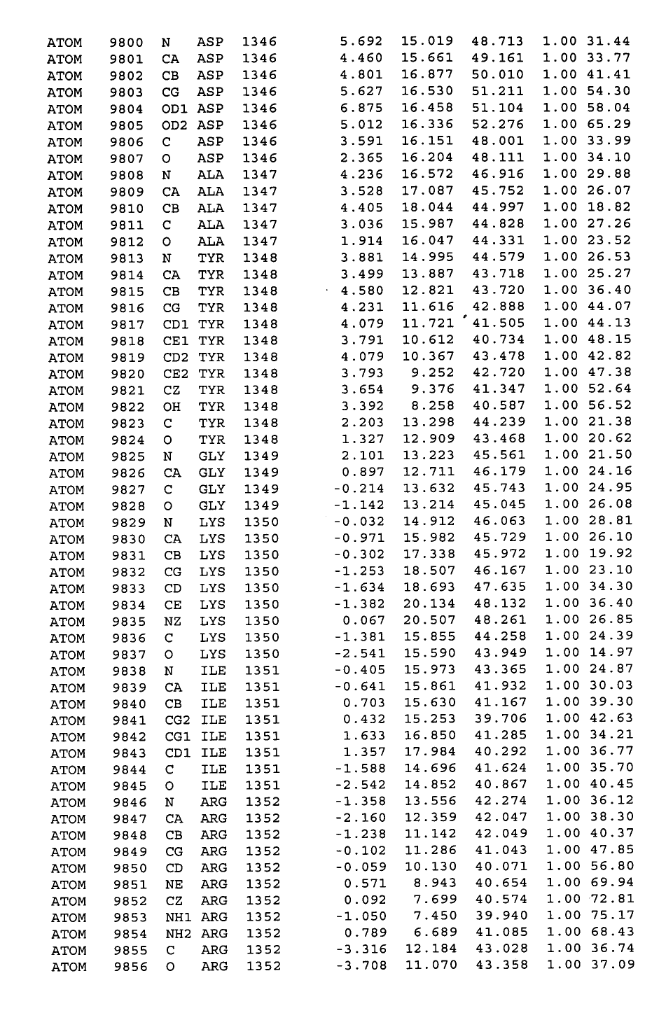

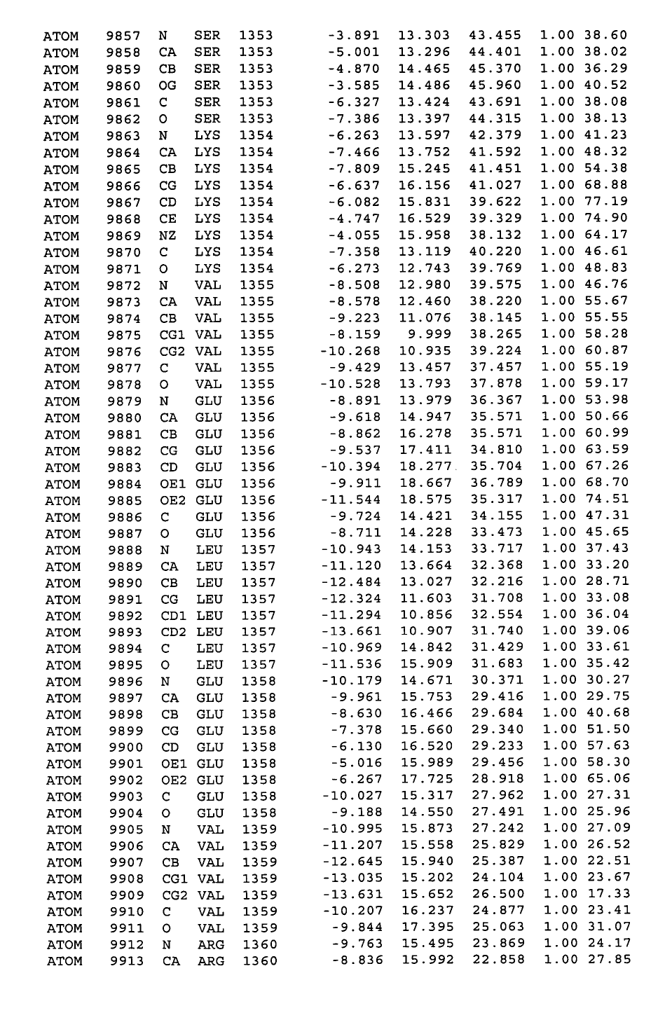

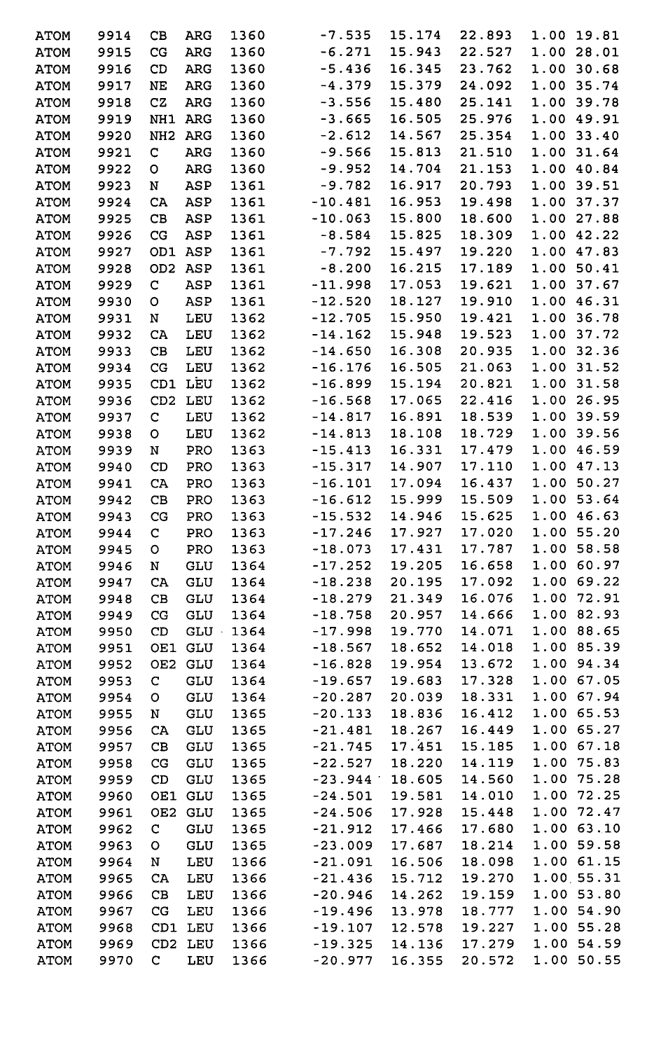

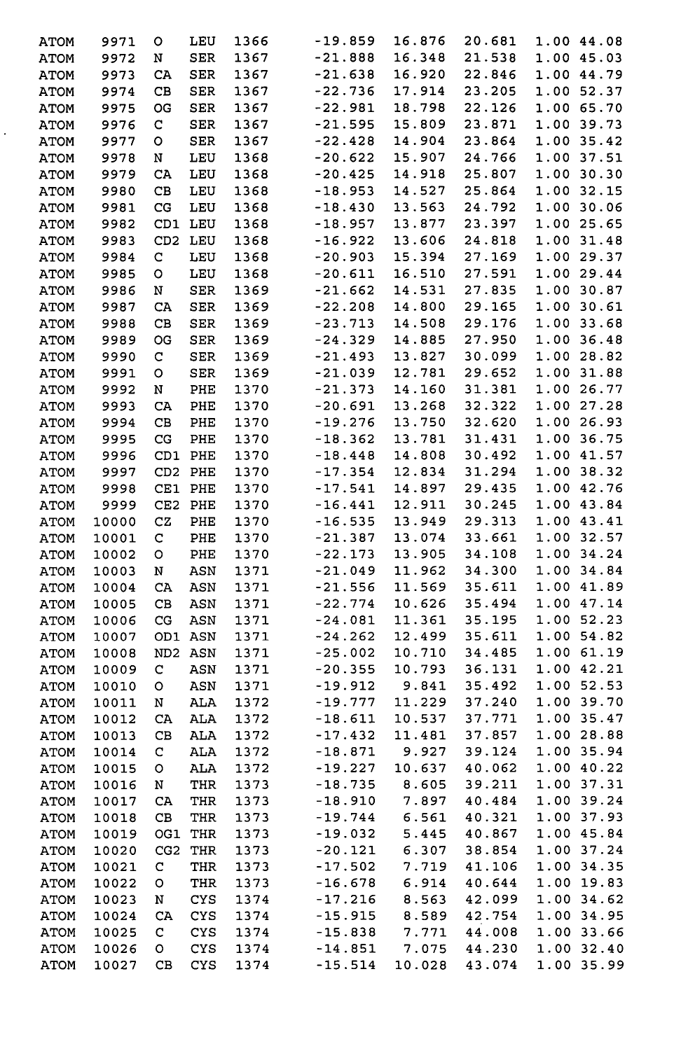

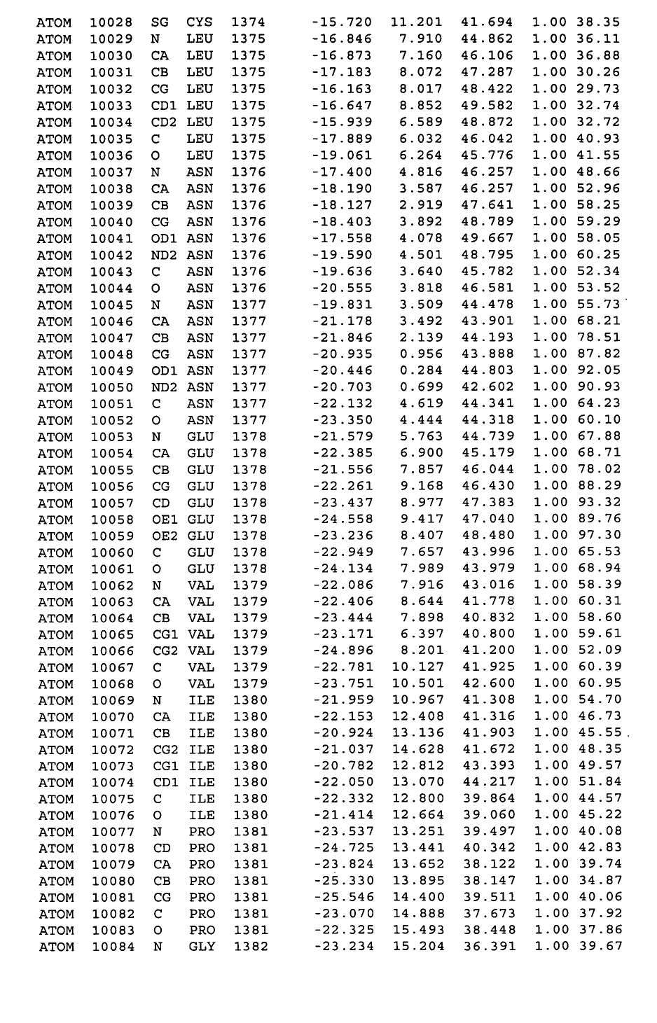

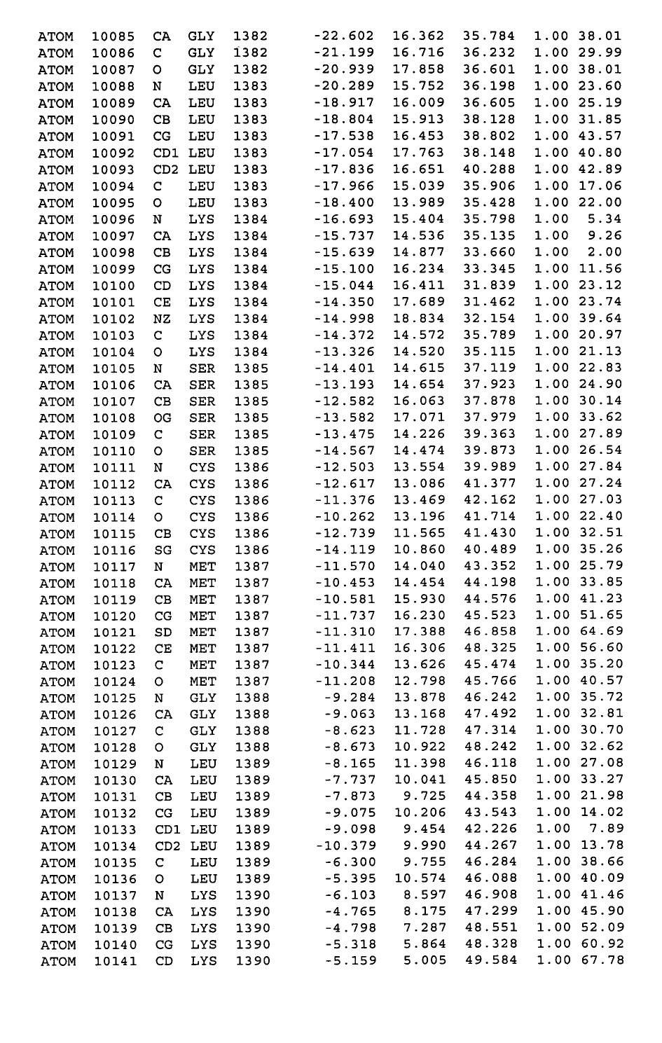

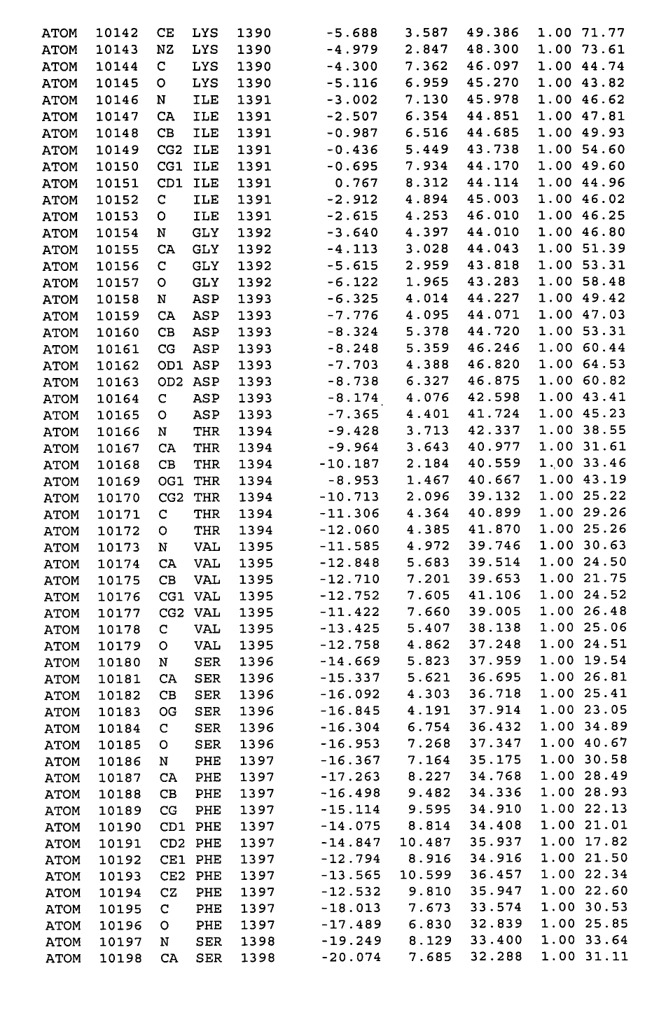

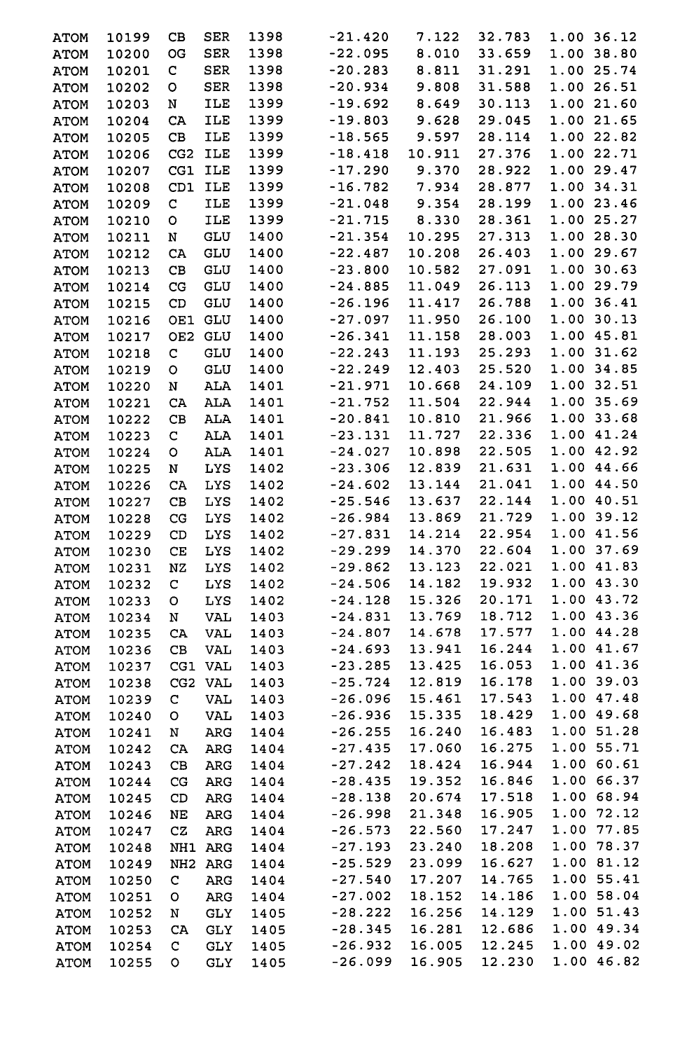

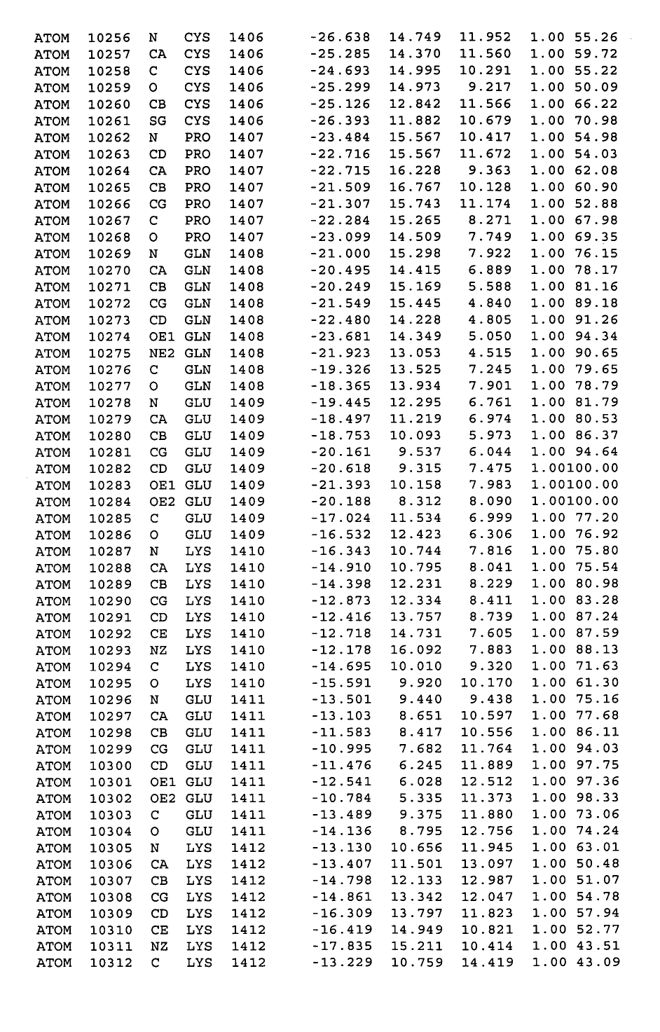

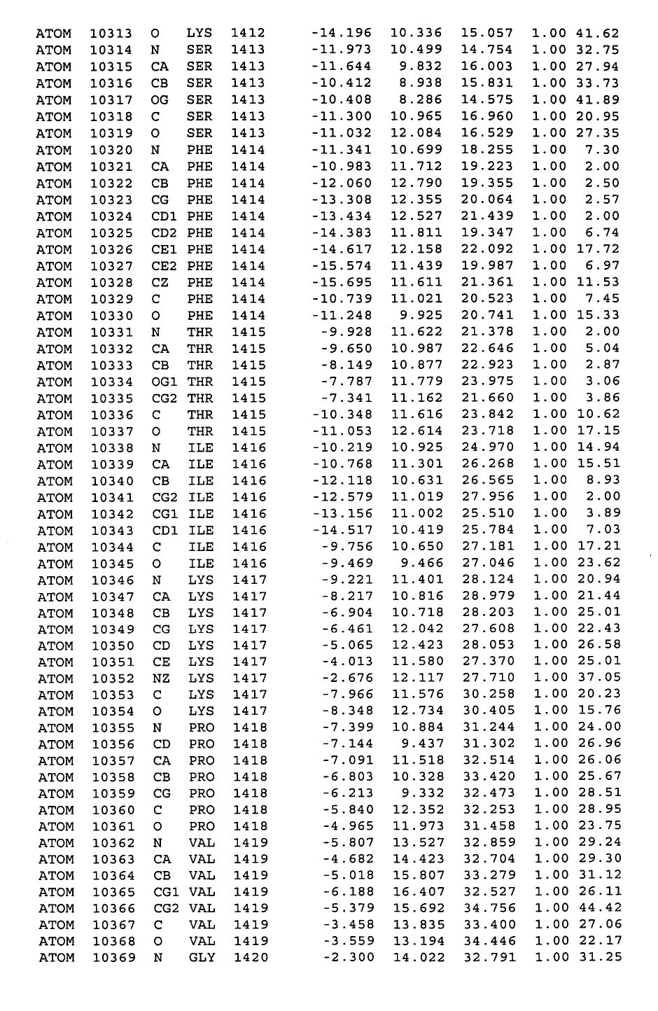

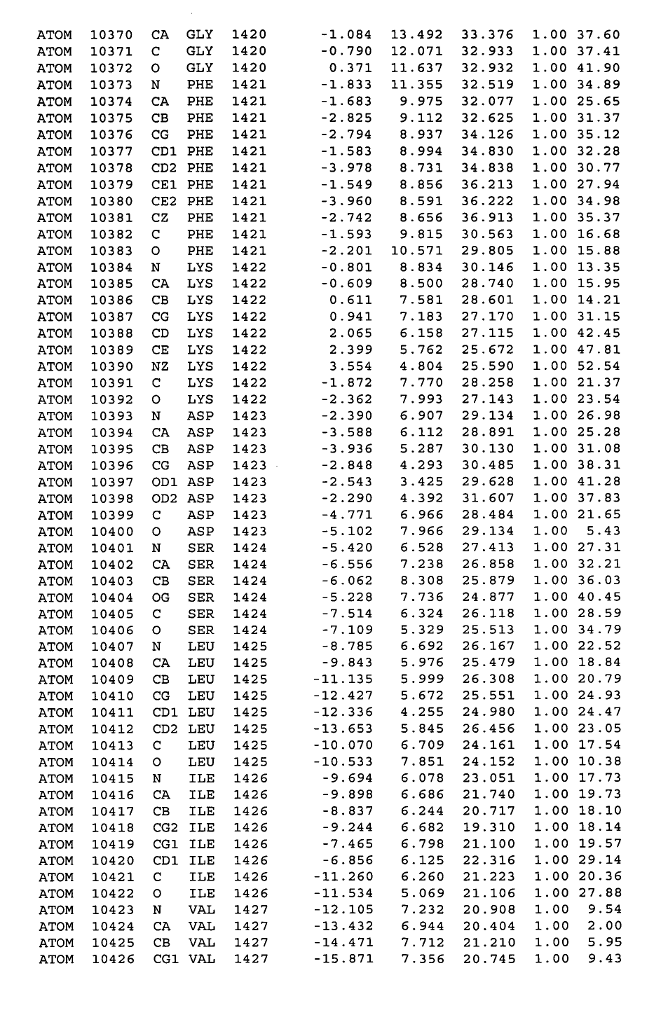

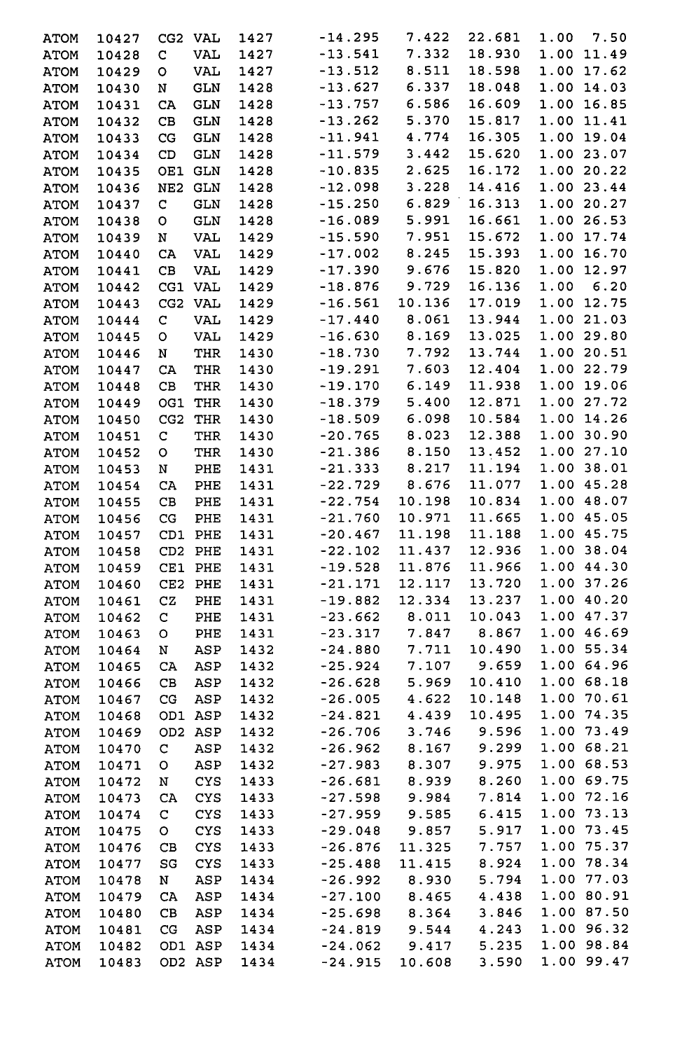

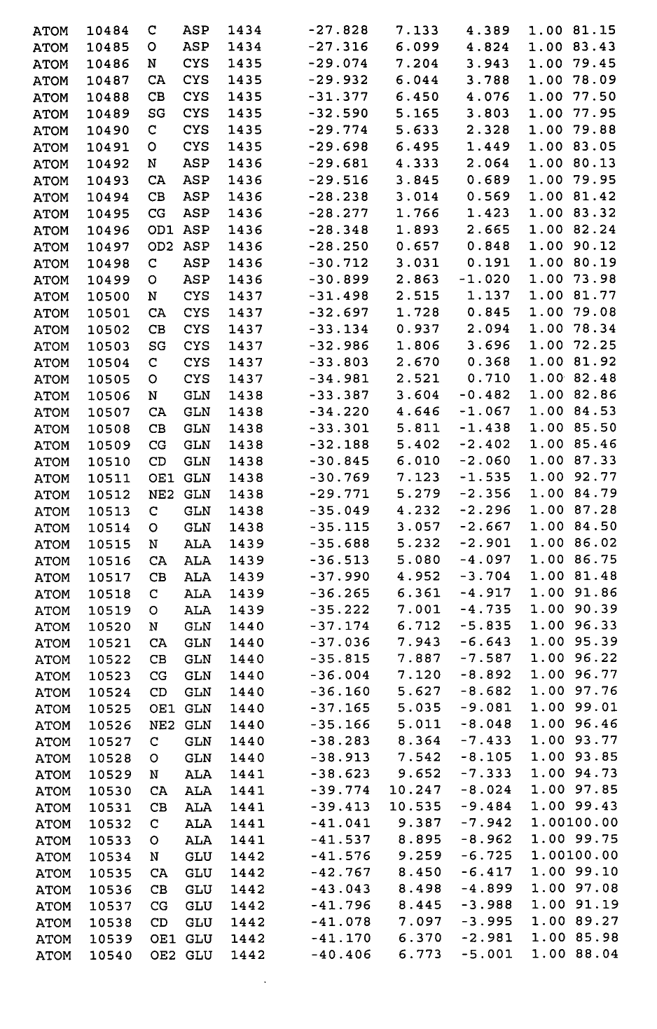

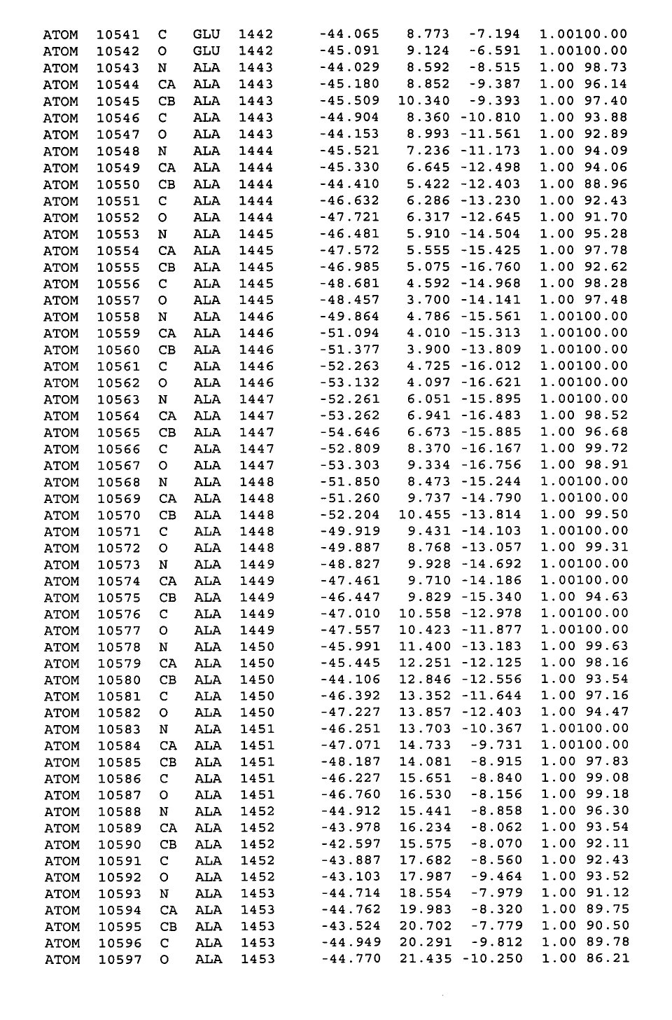

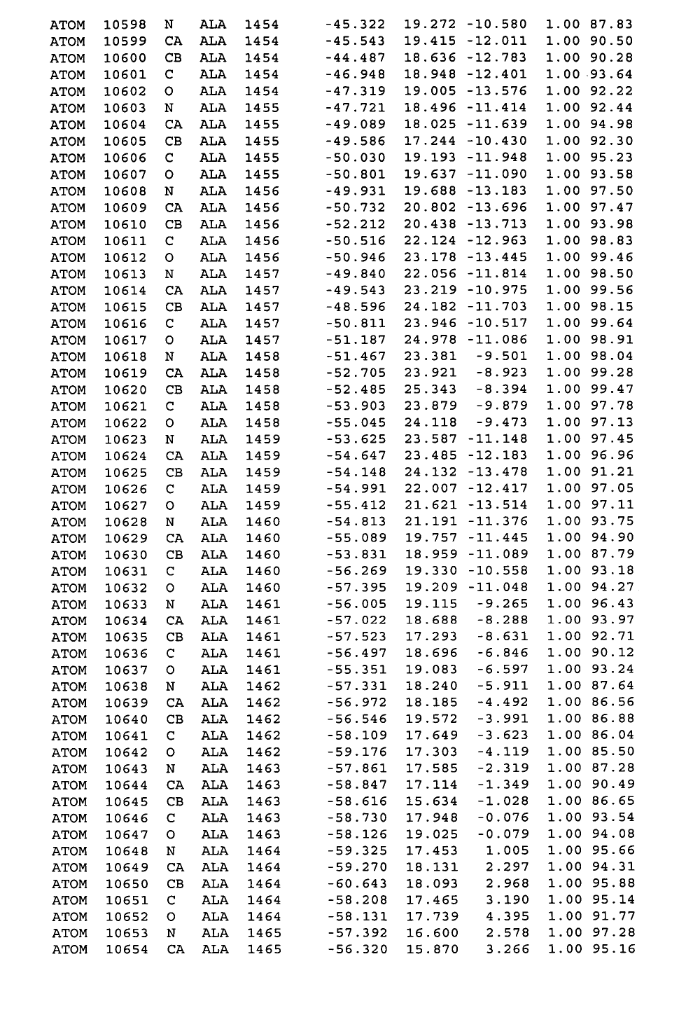

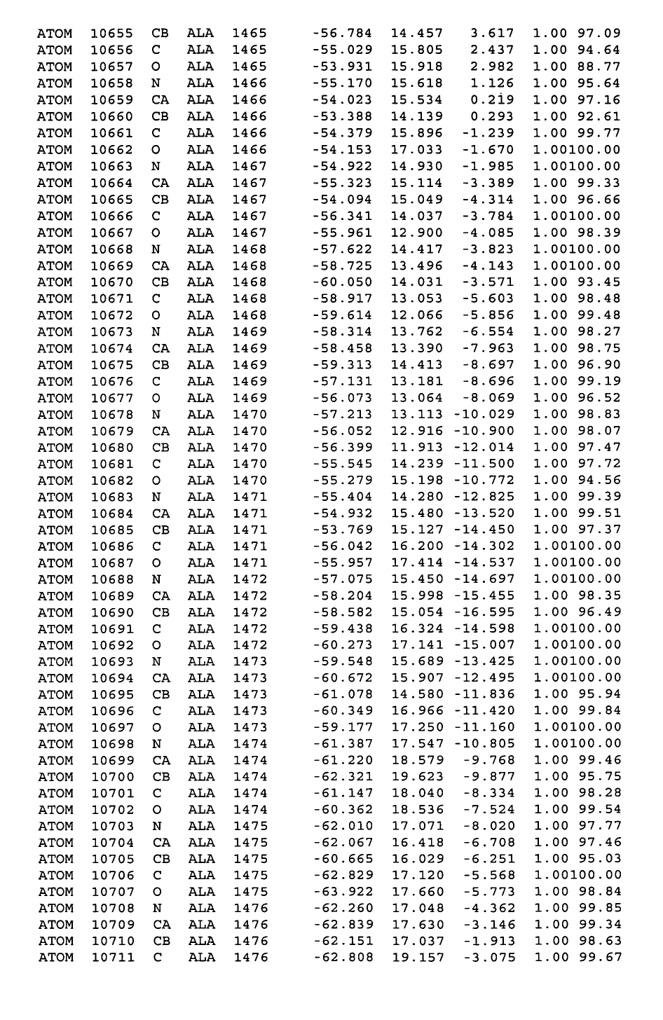

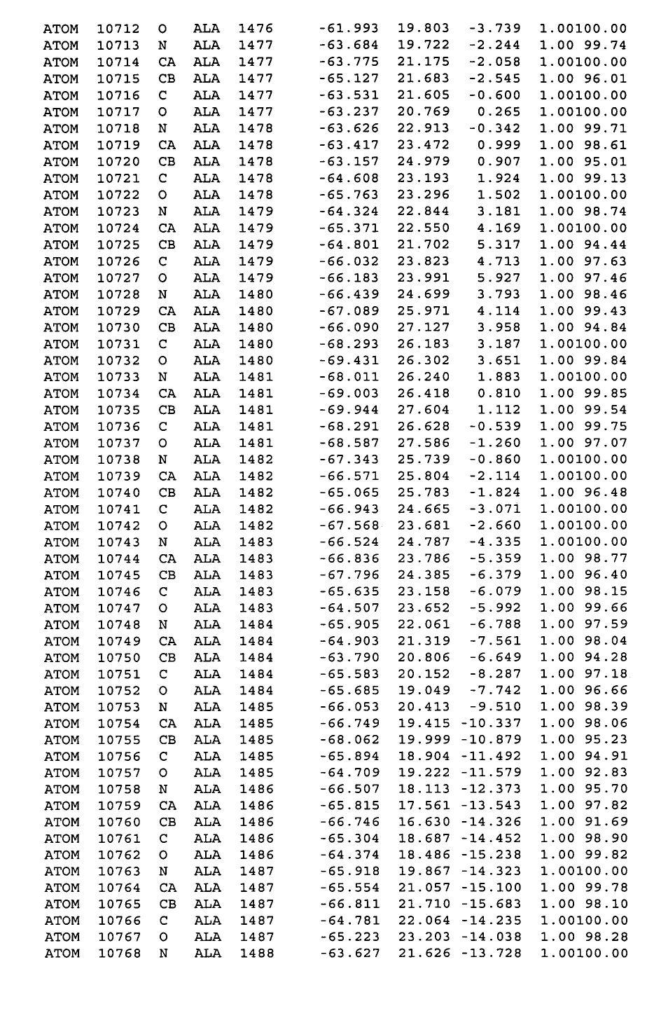

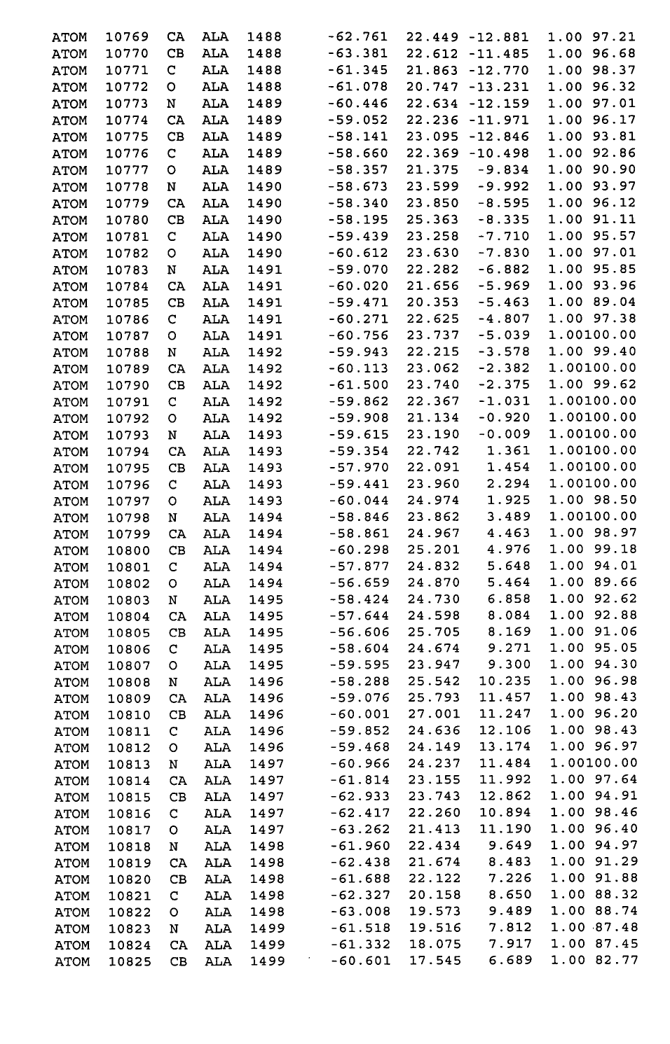

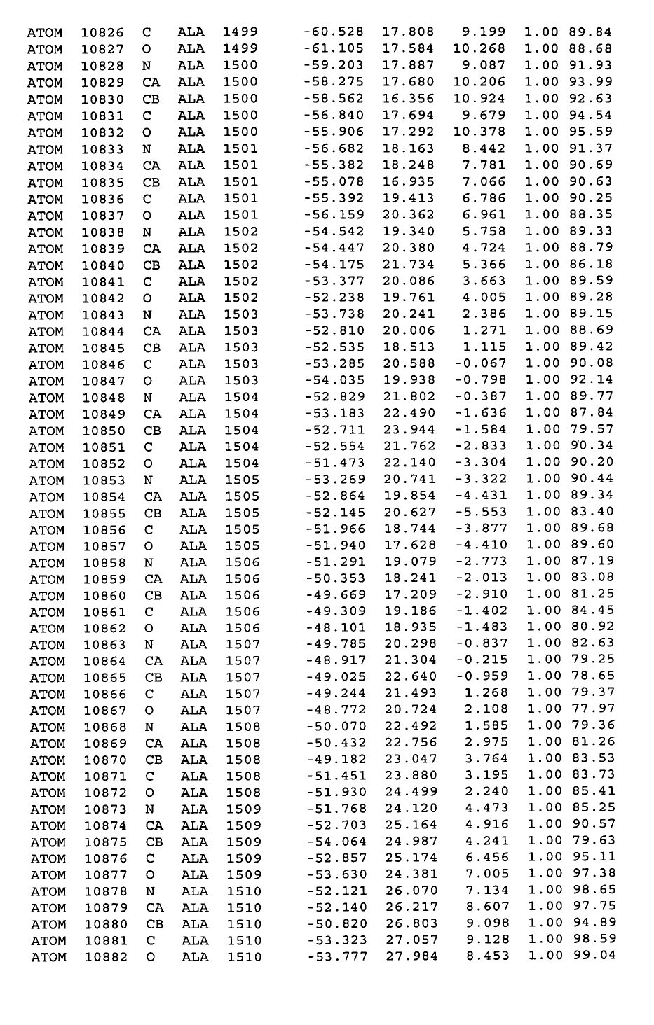

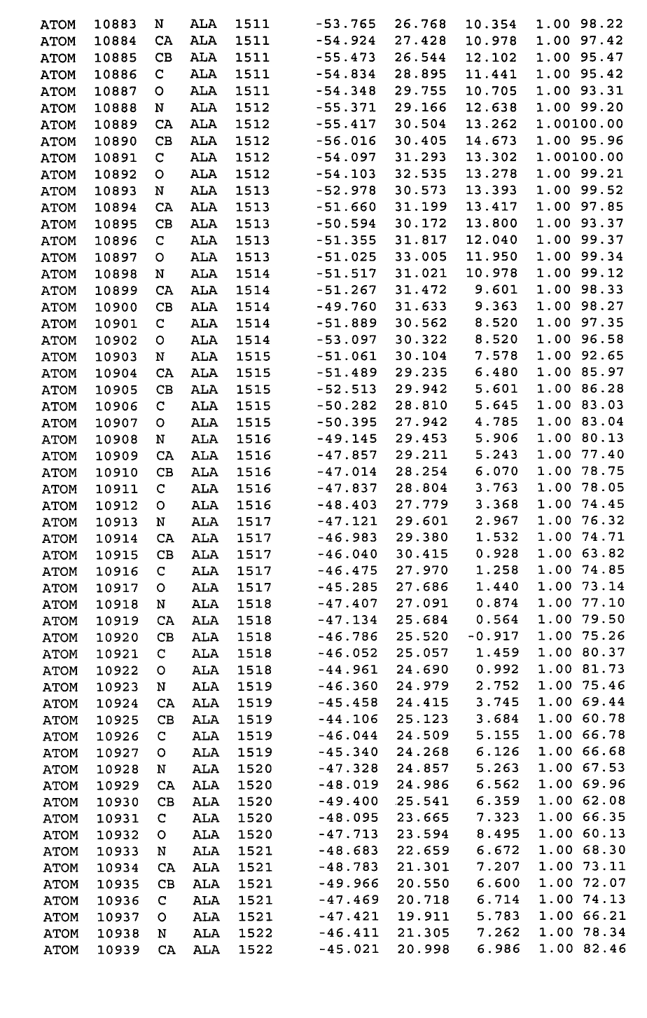

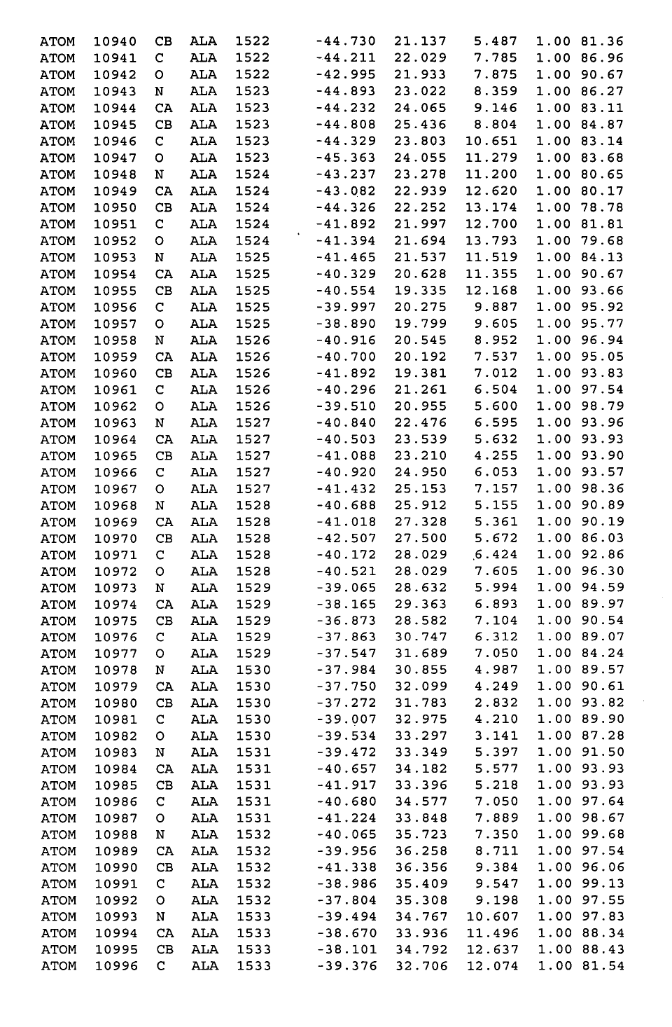

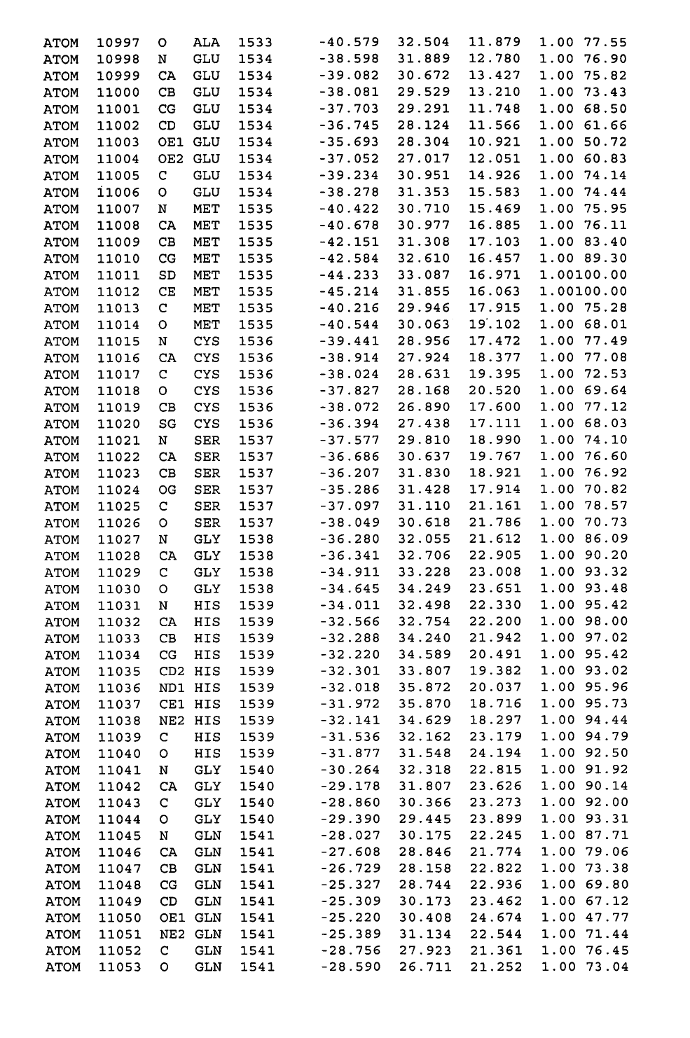

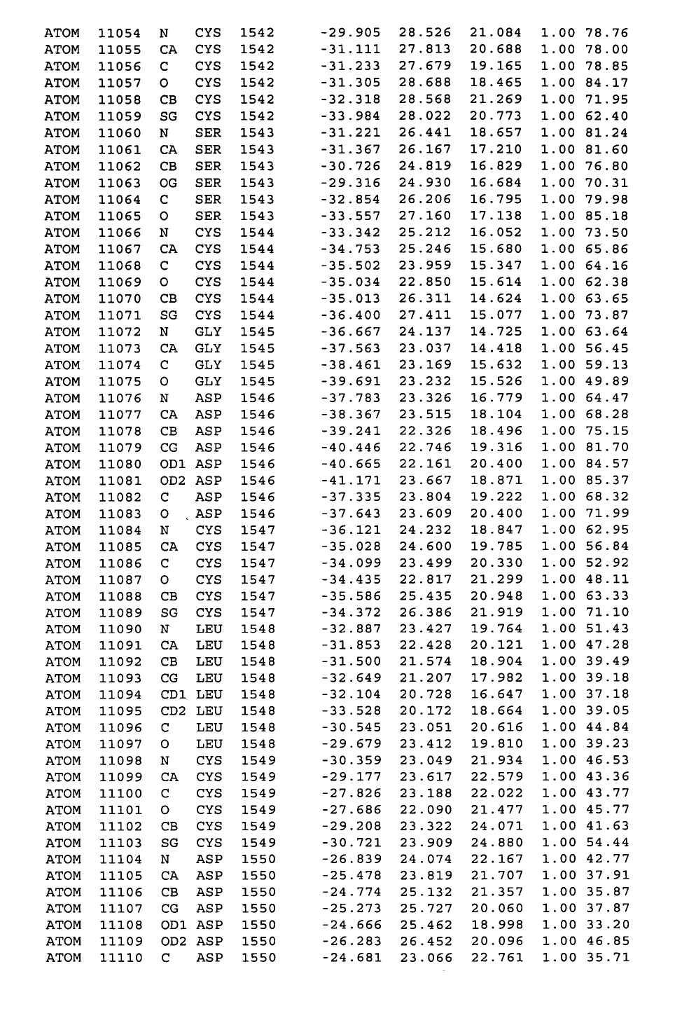

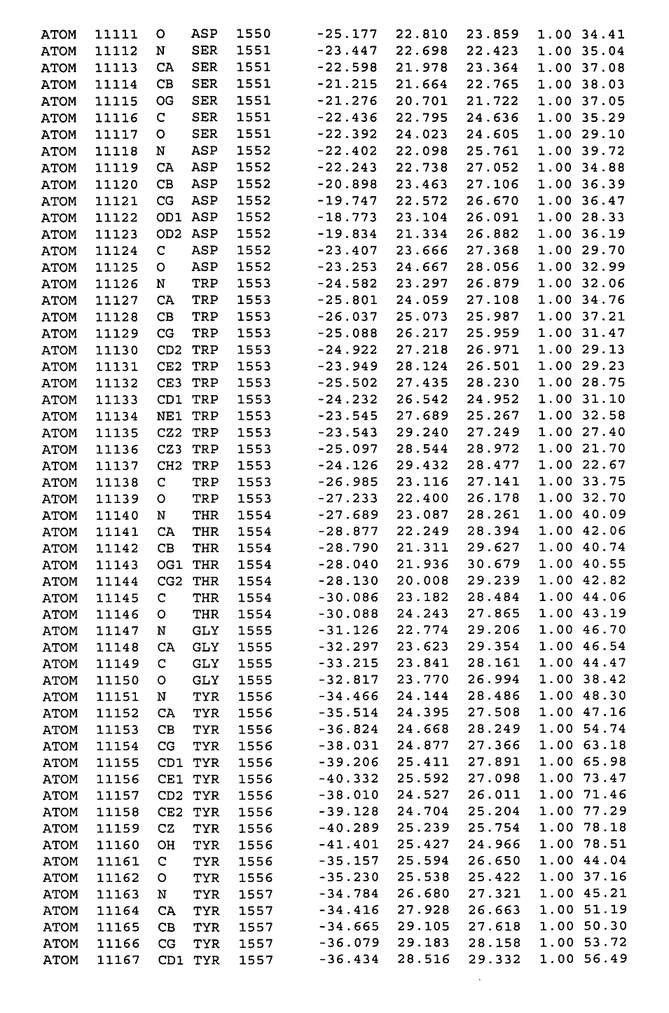

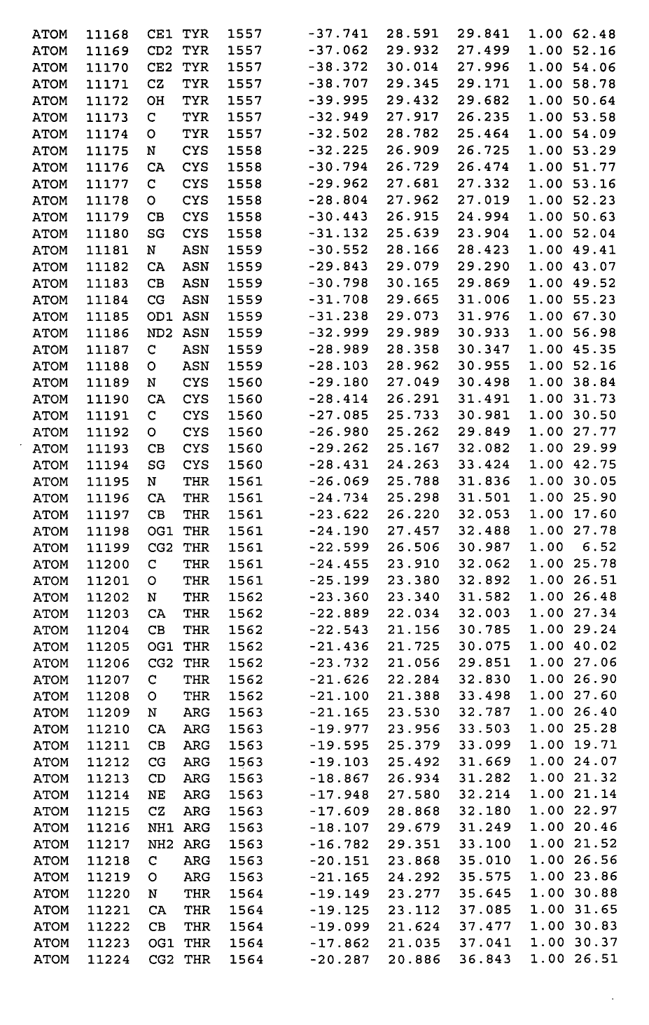

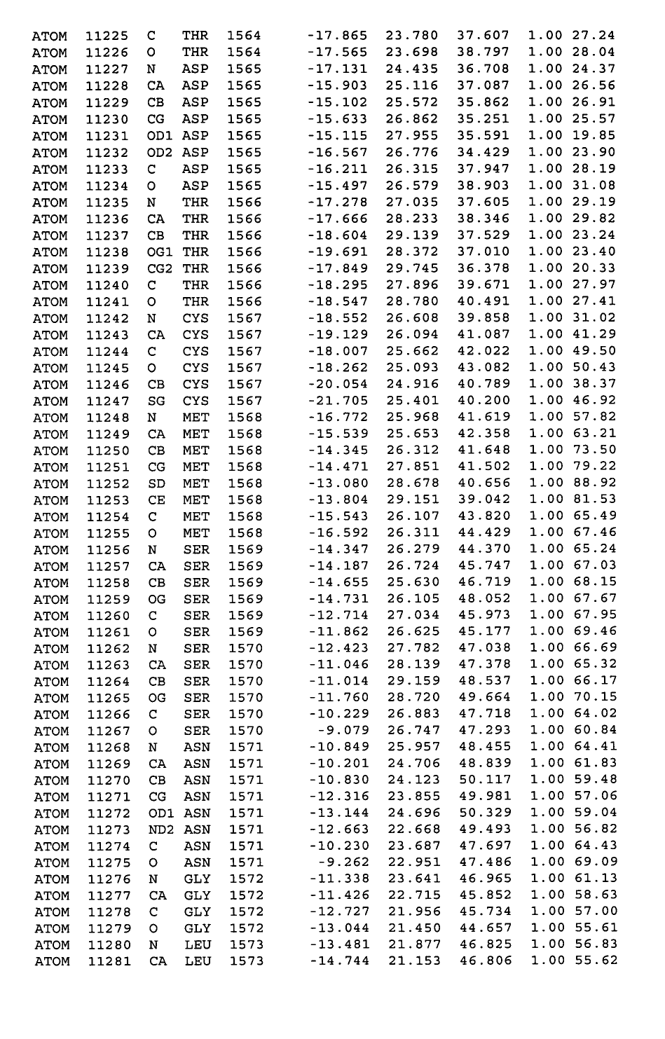

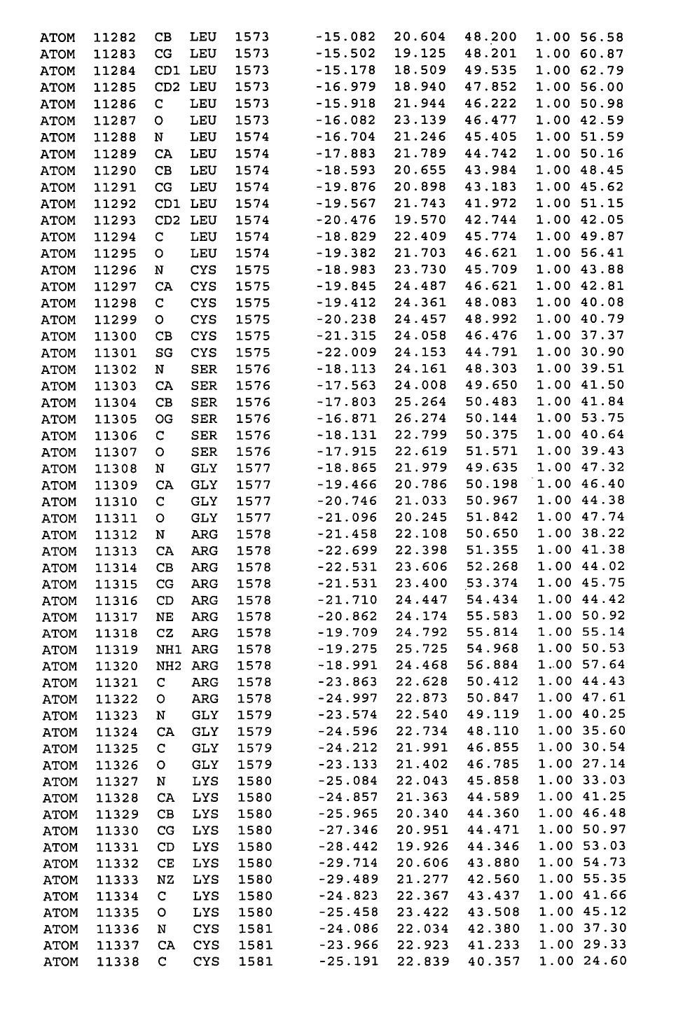

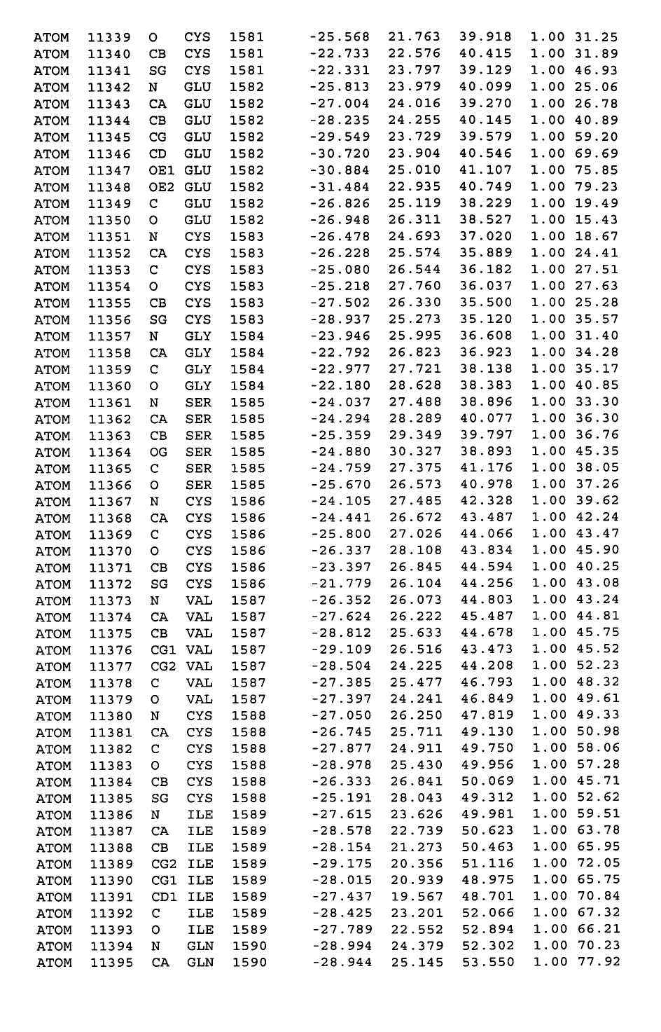

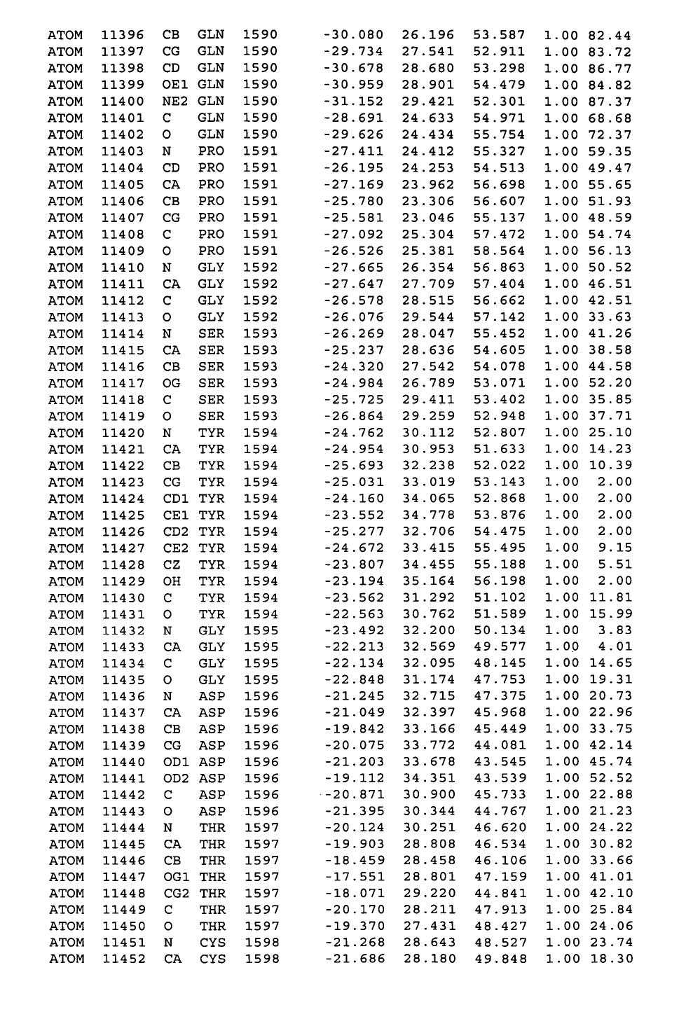

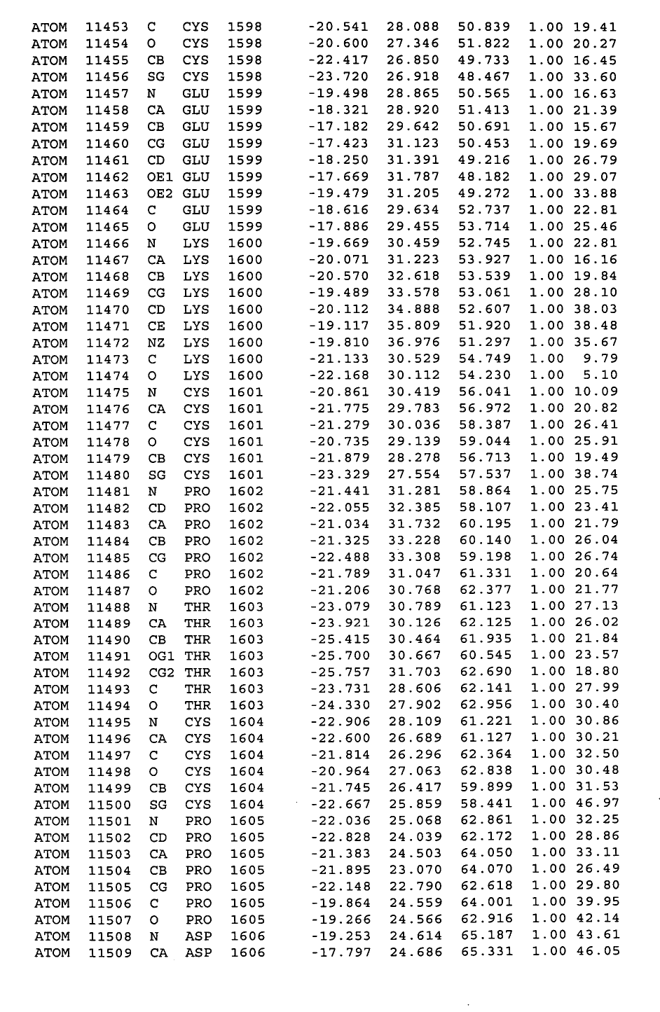

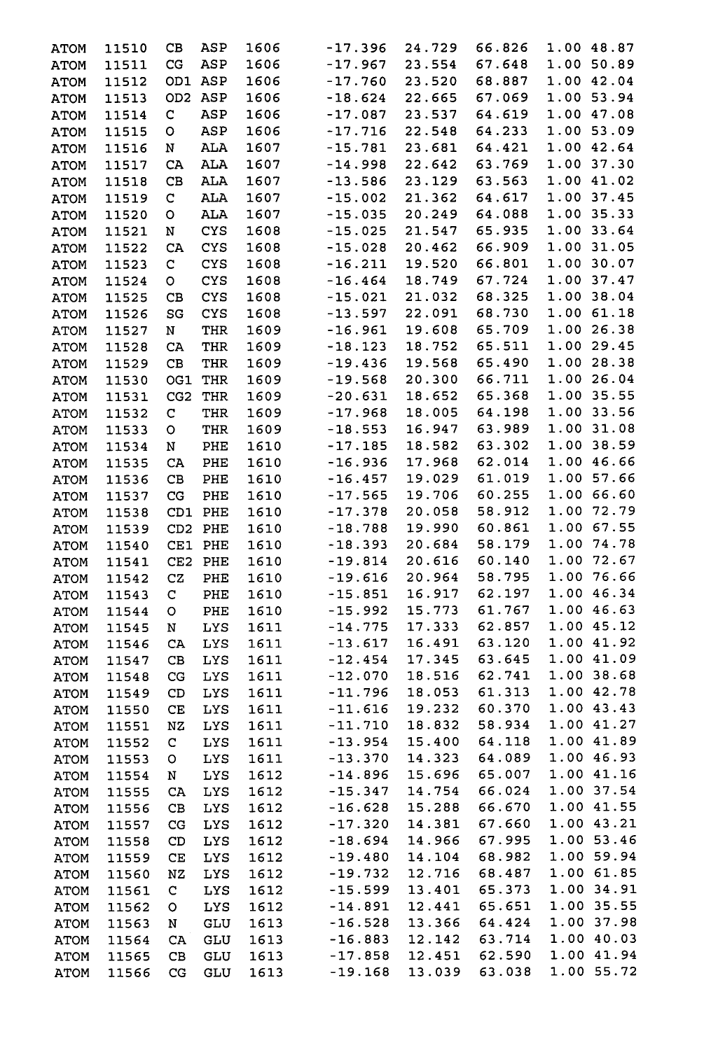

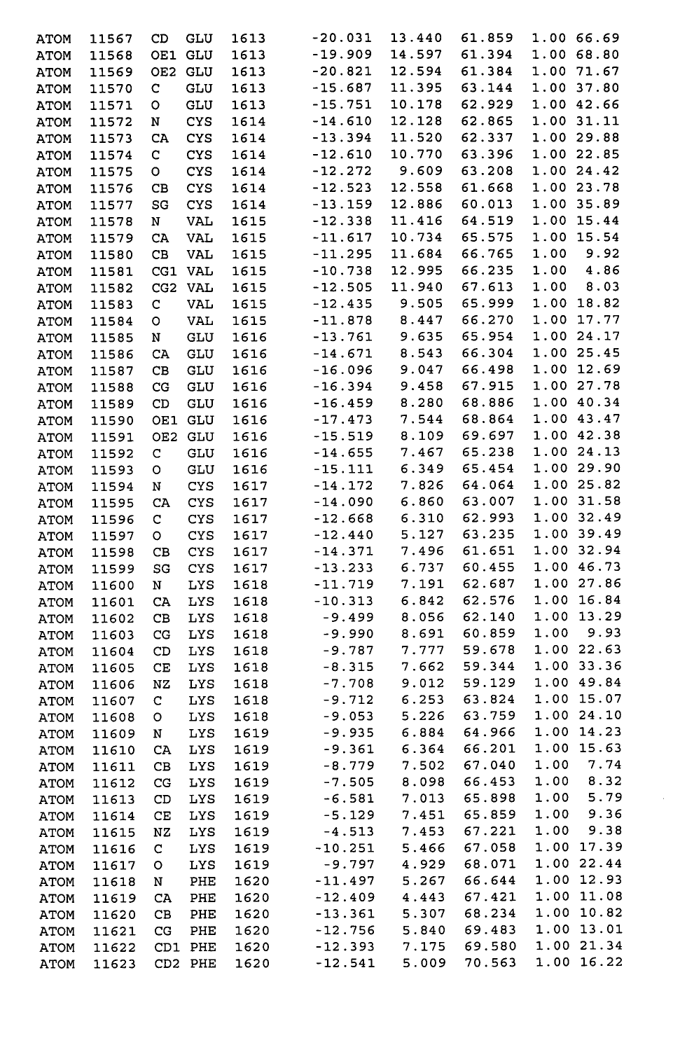

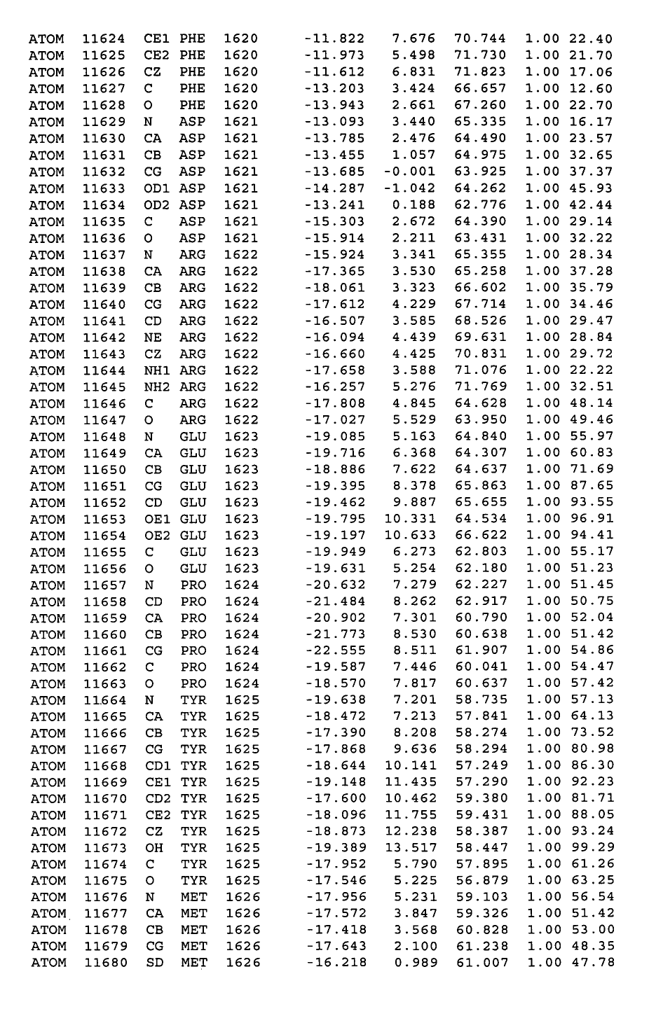

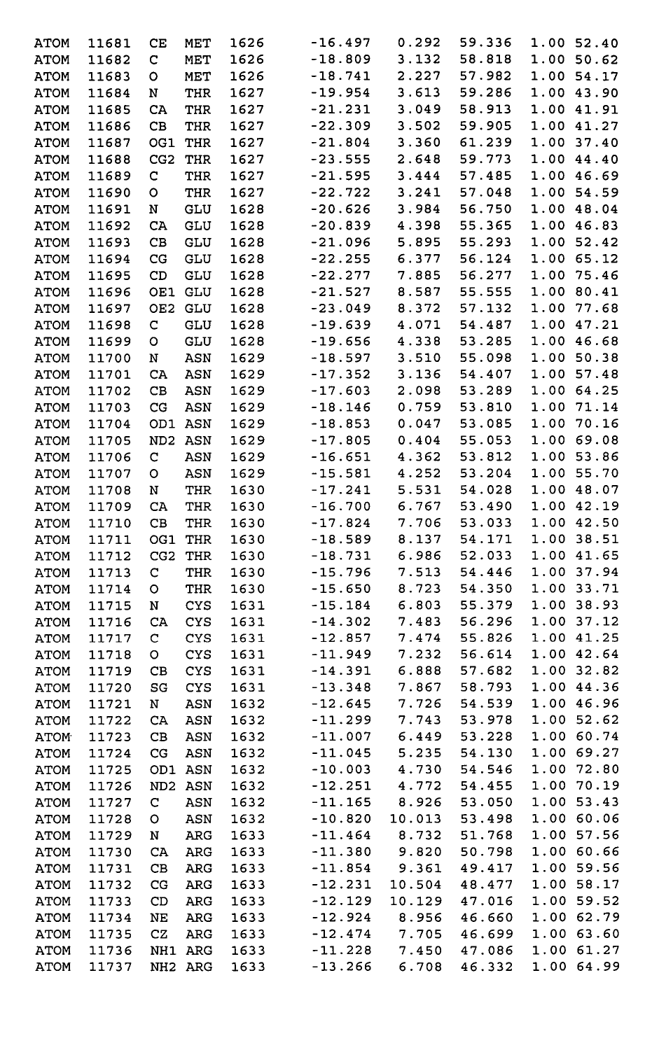

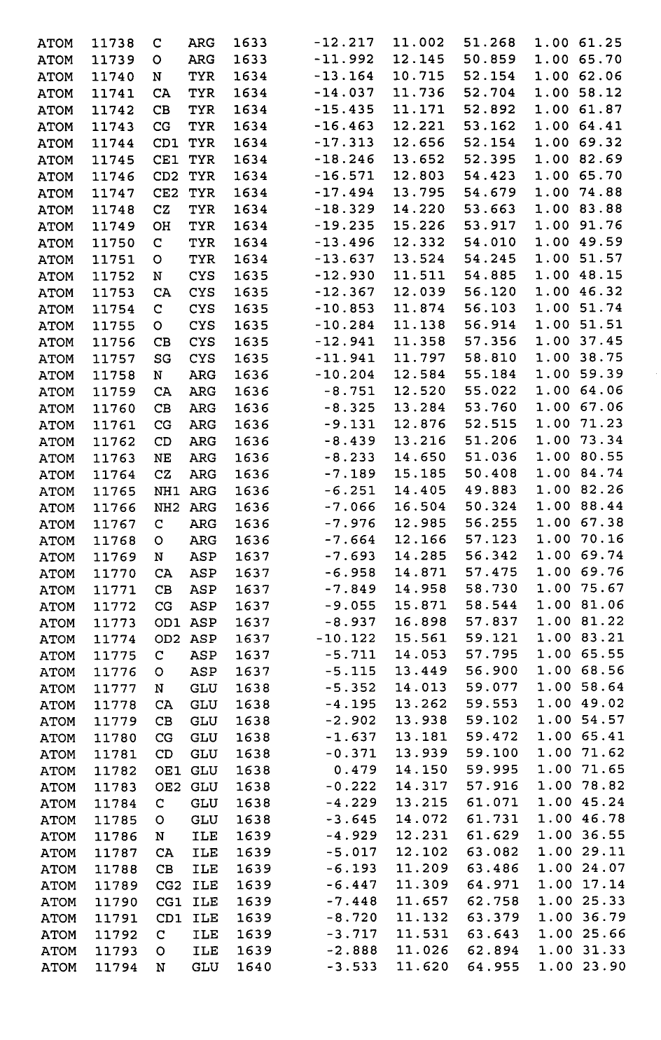

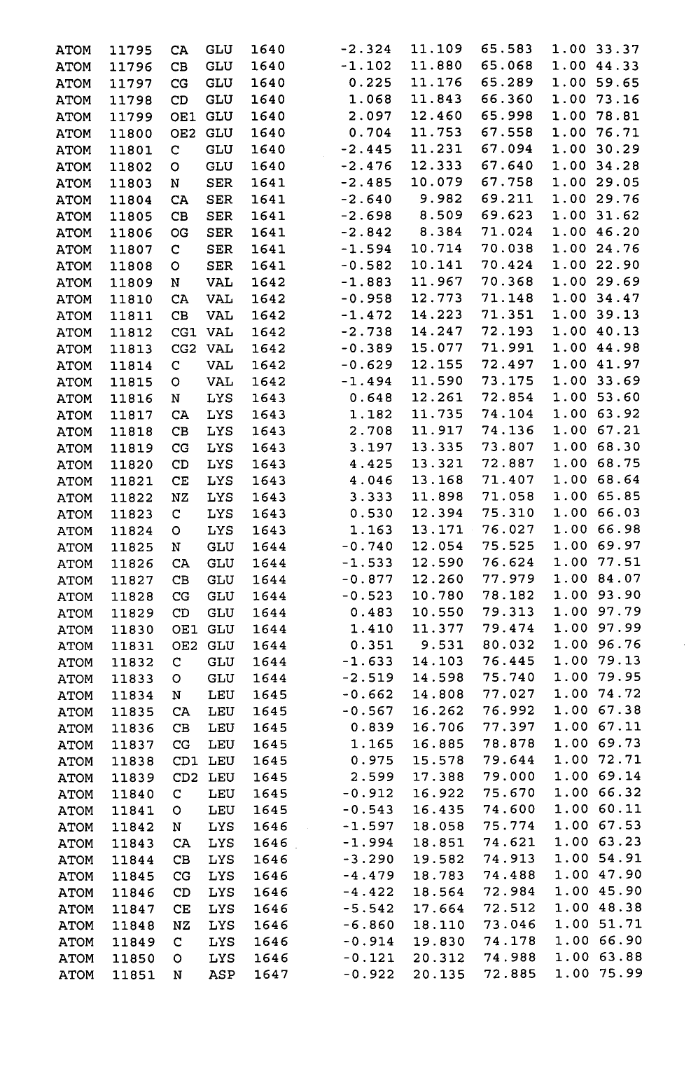

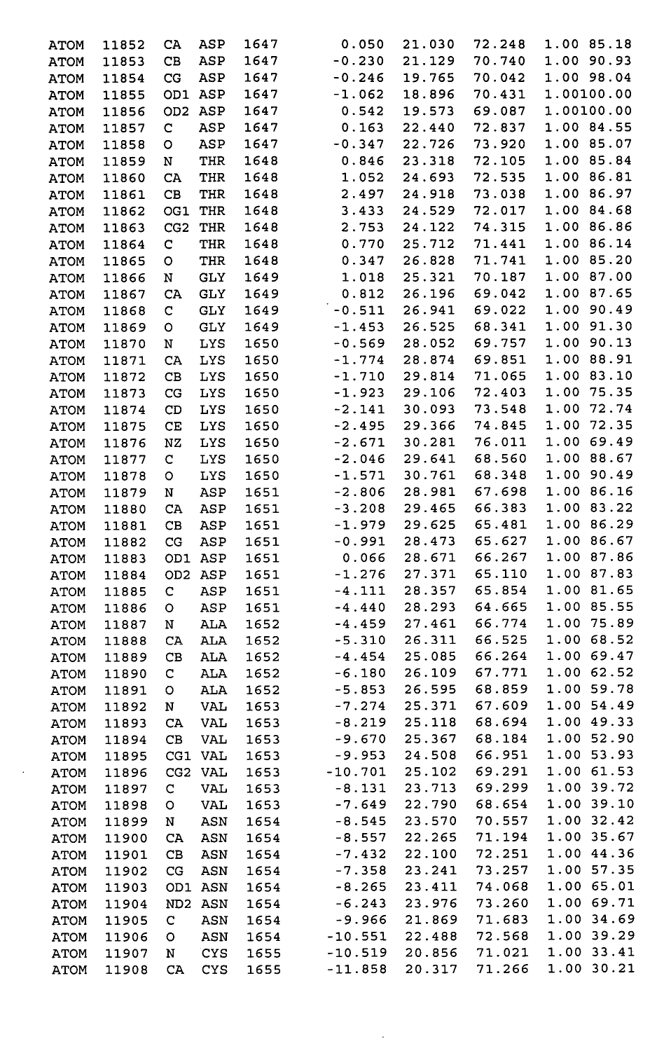

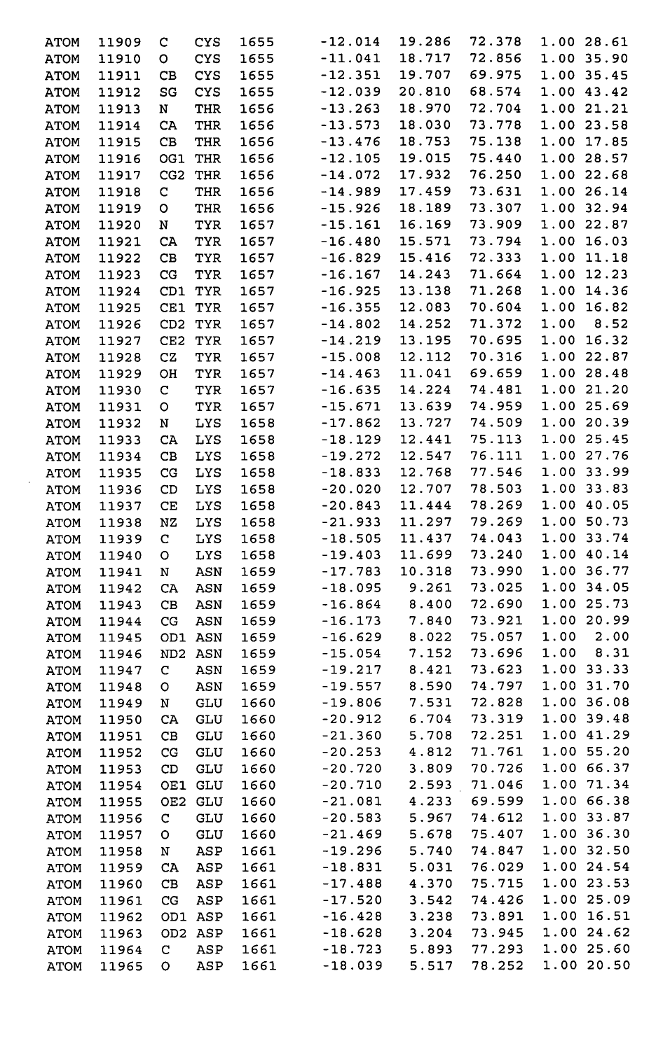

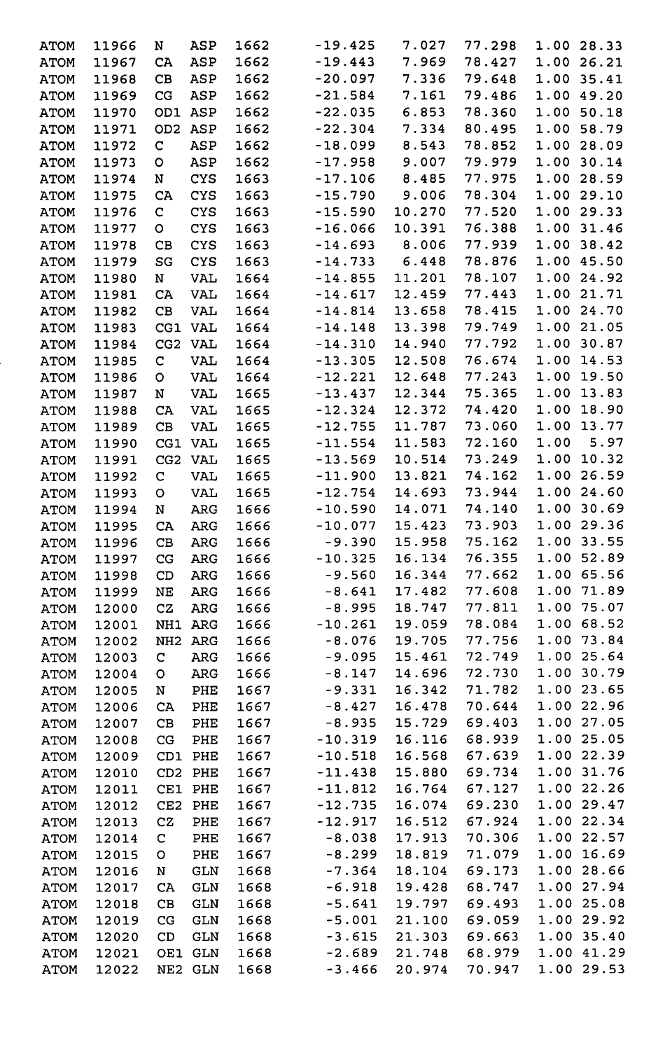

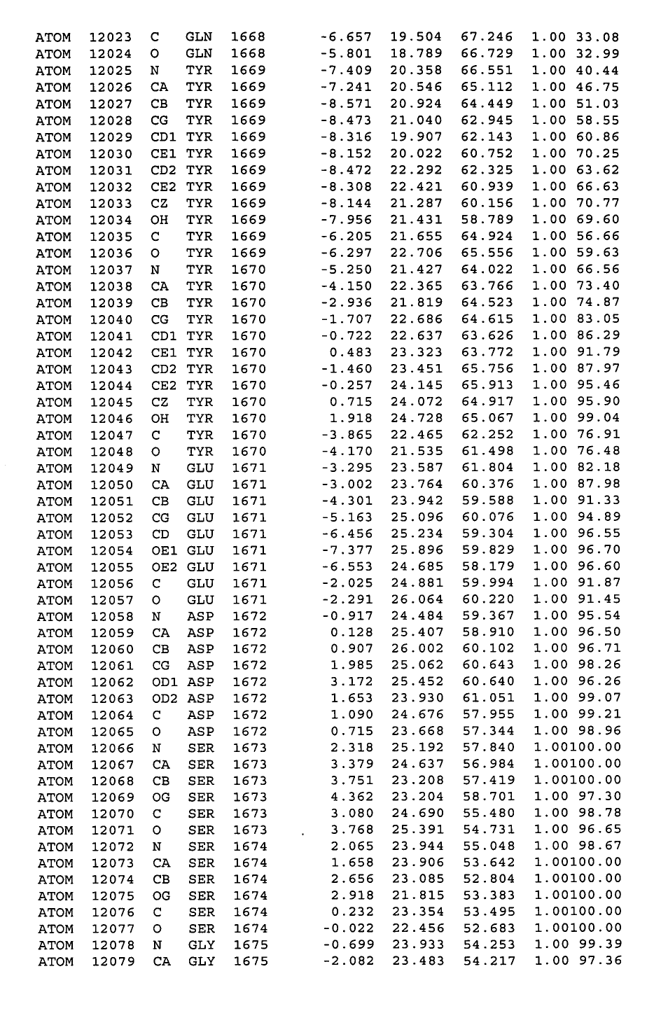

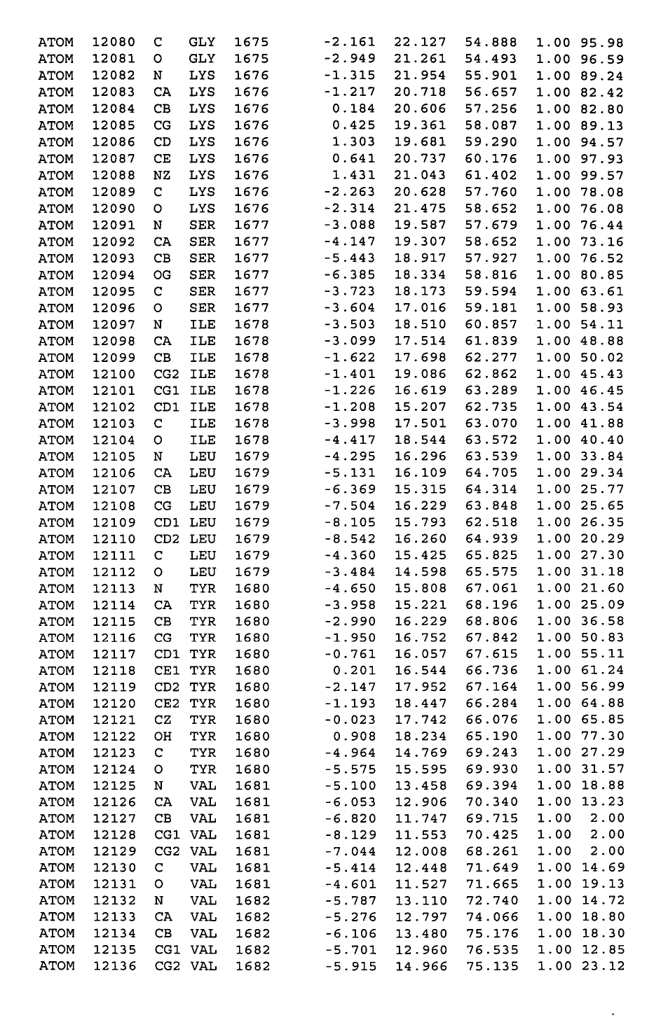

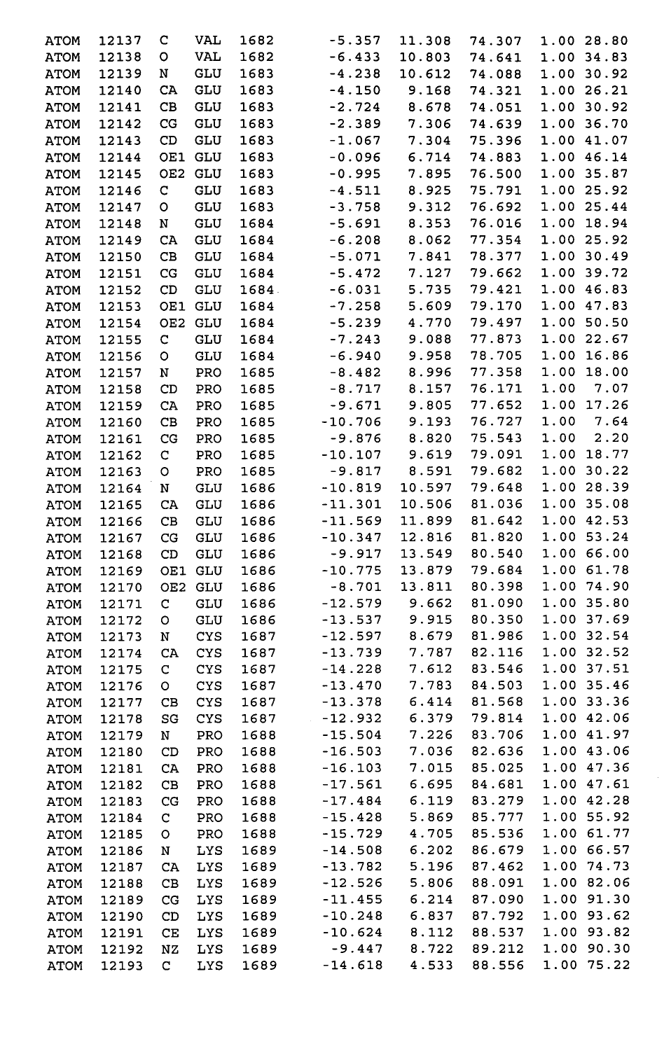

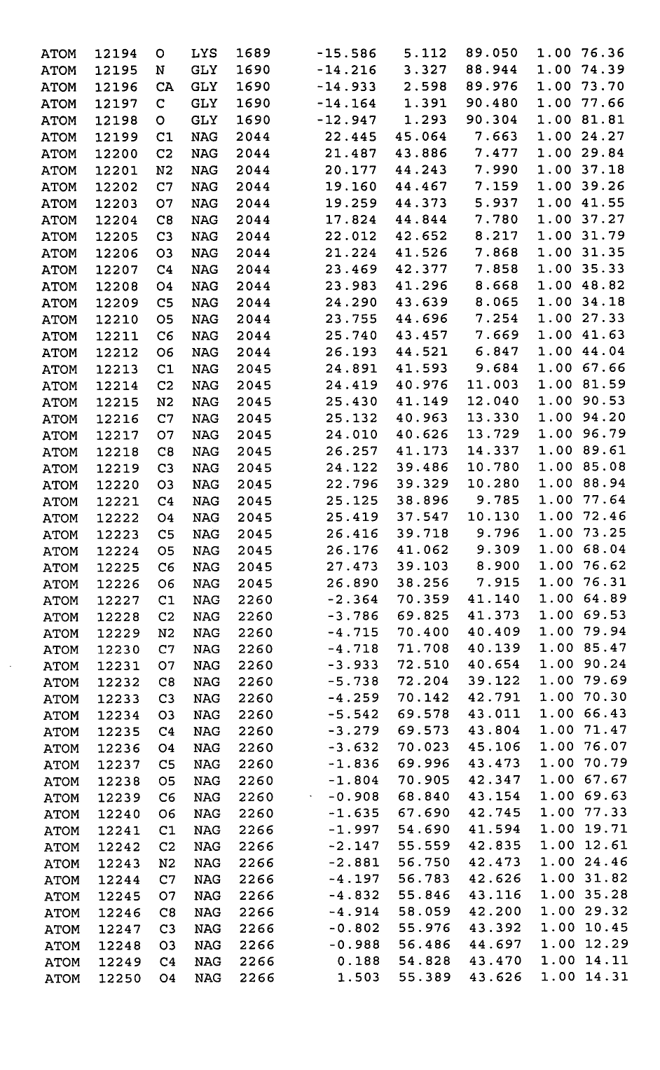

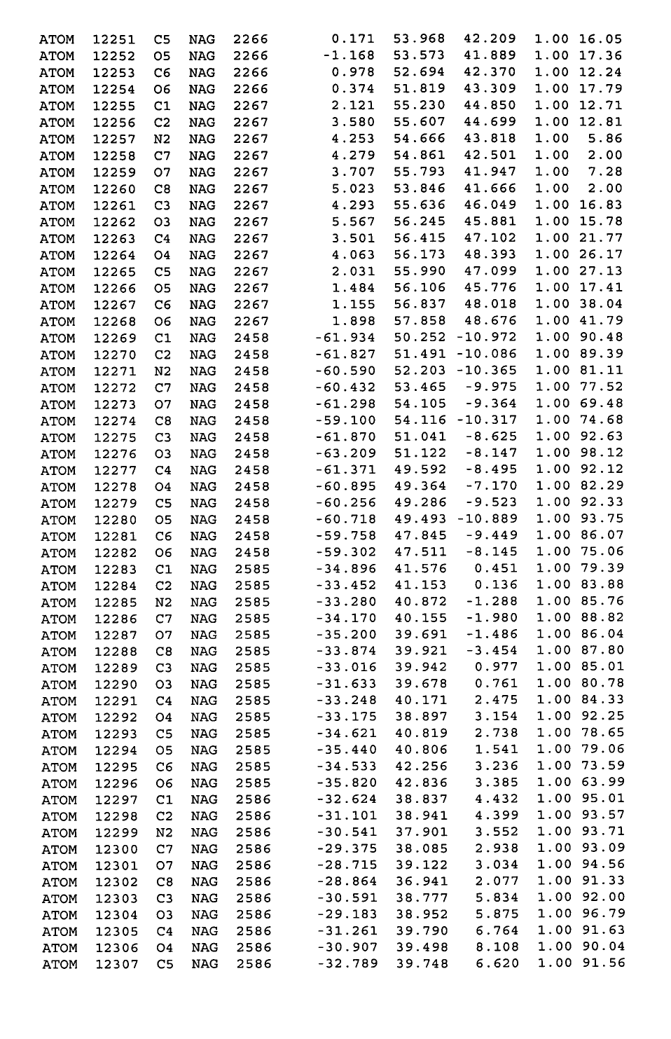

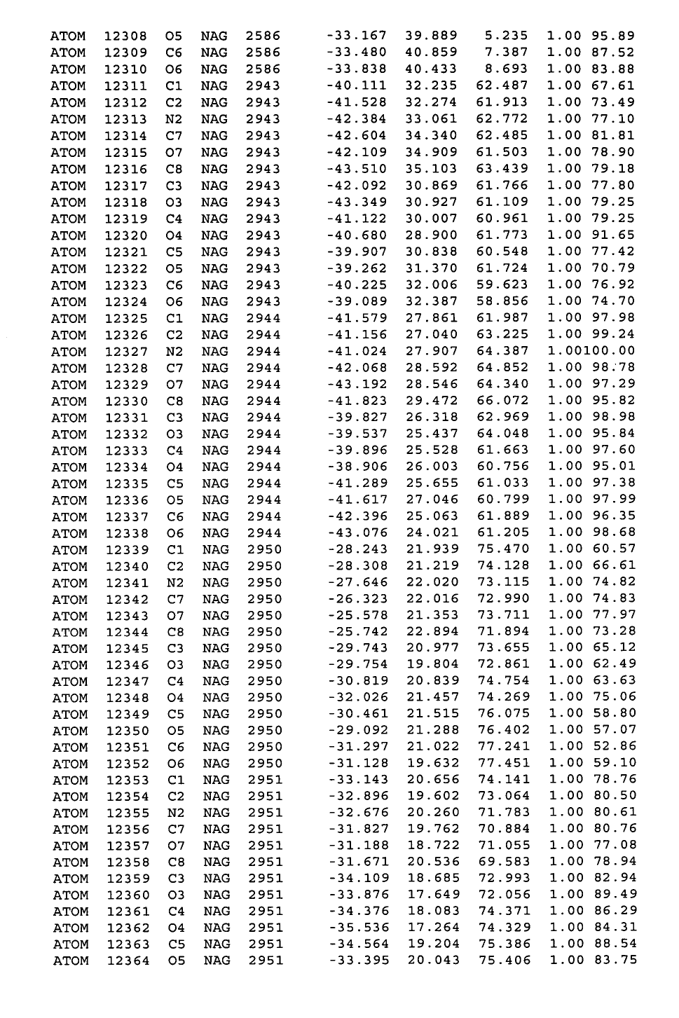

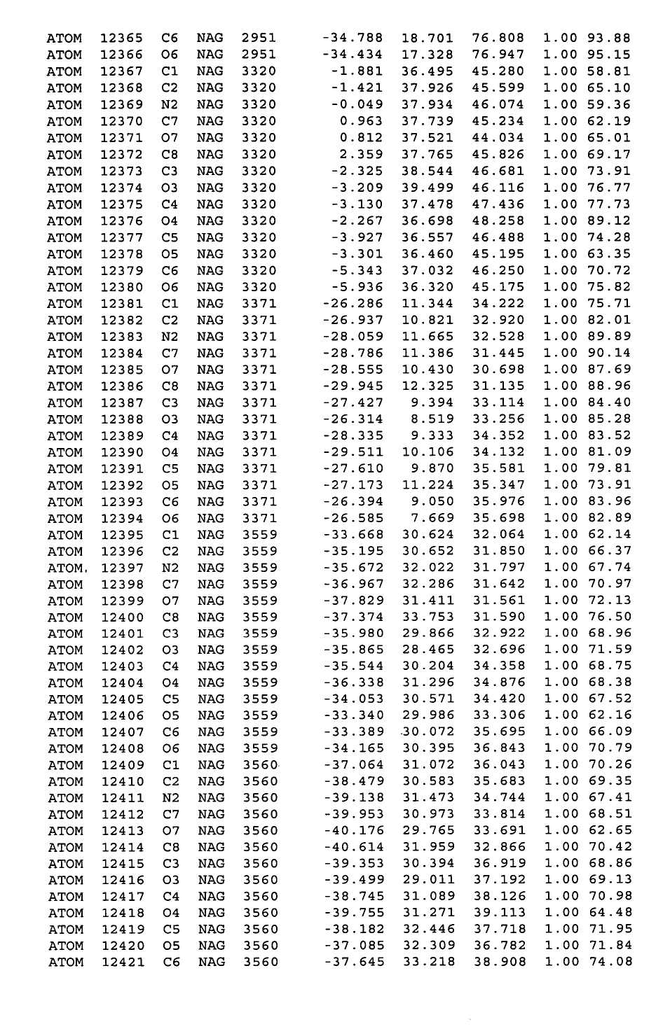

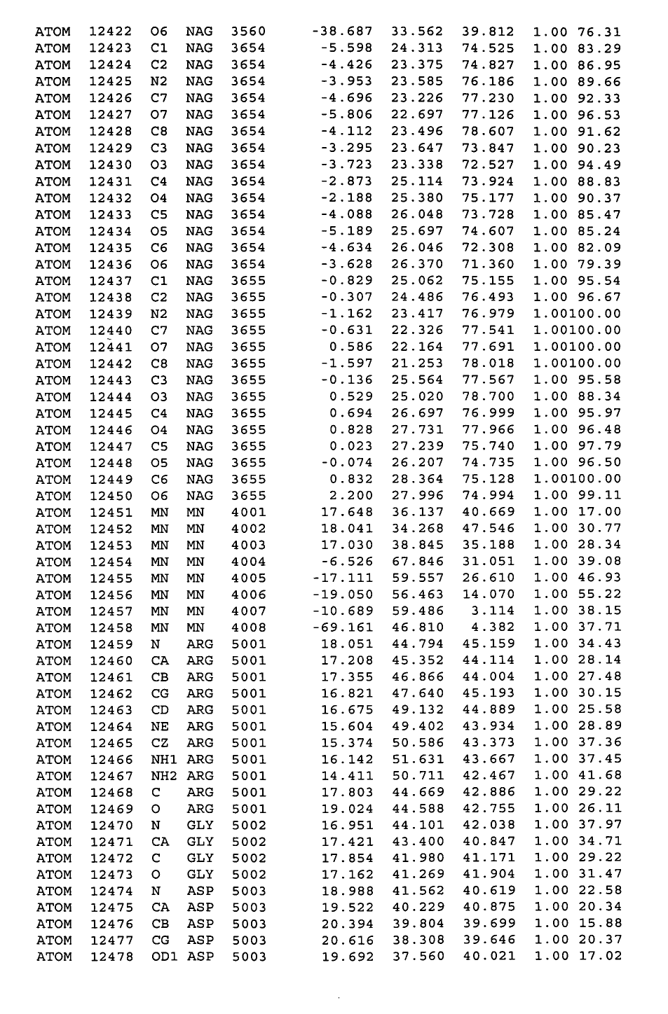

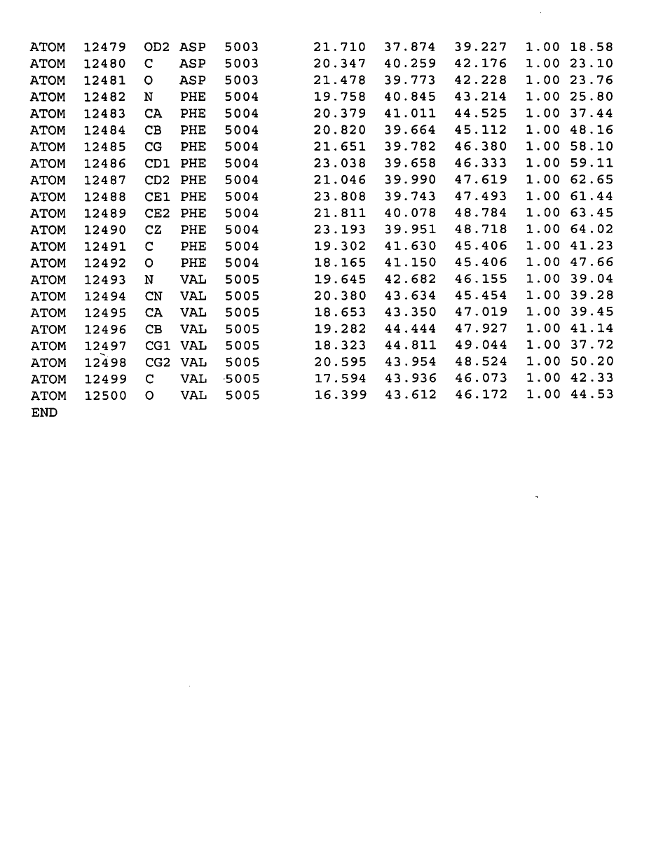

The invention is based, in part, on the identification of a mechanism for “deadbolt inhibition” of integrins and features methods for identifying compounds which bind to integrins (e.g., αVβ3), particularly compounds that inhibit activation of the integrin and inhibit ligand binding to the integrin thus providing deadbolt inhibition. The structural information in FIGS. 9 and 10 can be used to identify these various inhibitors of integrin (e.g., αVβ3) activity and ligand binding. Preferred mimetics and antagonists identified using the methods of the invention act to inactivate integrins in one or more in vitro or in vivo biological assay of the activity of an integrin (e.g., αVβ3).

The methods of the invention entail identification of compounds (e.g., small molecules naturally occurring polypeptides, and non-naturally occurring peptides or polypeptides (e.g., peptoids, peptidomimetics, antibodies, and the like) having a particular structure. The methods rely on the use of precise structural information derived from x-ray crystallographic studies which include the βA domain of an integrin (e.g., αVβ3). These crystallographic data permit the identification of atoms in the compound (e.g., naturally occurring and non-naturally occurring polypeptides, small molecules, and the like) that are important for integrin binding and integrin modulation (e.g. inhibition). More importantly, these data define a three-dimensional array of the important contact atoms. Other molecules which include a portion in which the atoms have a three-dimensional arrangement similar to some or all of these contact atoms are likely to be capable of binding an integrin and acting as an integrin modulator (e.g., inhibitor). The methods can also employ isolated integrin βA domains defined on the basis of their X-ray crystallographic or other high-resolution molecular structural data (e.g., via solution NMR), or on the basis of their homology to the structure of integrin αVβ3 as isolated domain or as fusion partner (e.g., with the Fc domain of immunoglobulin), and use conventional inhibitor screens based on the inhibition or stimulation of interaction of the βTD domain with the βA domain.

A recent study, based on analysis of negatively-stained EM (electron micrograph) images of extracellular integrins in solution, found that Mn²⁺ and RGD (arginine-glycine-aspartate) elicit global changes in the integrin, causing it to switch from the bent to the knee-straightened form, regardless of whether its legs are chemically tied²⁴. Straightening at the knees was proposed to be caused by an outward swing of the hybrid domain relative to βA, driven by a downward slide of the βA's C-terminal α7 helix. Since the bent form of extracellular or membrane-bound αVβ3 (both produced by locking the head to the legs through a disulfide bond introduced based on the αVβ3 atomic structure) was found inactive but could be activated by chemical reduction of disulfides²⁴, it was proposed that this “switchblade” model underlies integrin activation. While appealing, this model does not explain a number of observations. First, inside-out activation is rapid (<1 sec) and reversible. The switchblade model does not explain how the extended structure refolds within seconds, especially within the crowded confines of the extracellular matrix. Second, movement at the knees alone is insufficient to extend the 20 Å cryoEM structure of the native inactive αIIbβ3⁴⁵. Third, analysis of mAb epitope maps of active an inactive native αIIbβ3 suggest that both can be in the compact (bent) form⁸². Fourth, activation and extension can be dissociated: extracellular α5β1 tied at its legs can assume the straight conformation when unlocked, yet it is inactive⁷². Furthermore, fixing the structure in its bent unliganded state by artificial disulphides is expected to keep it unliganded, as tertiary and quaternary changes of the integrin are needed and occur even in the protein crystal lattice to allow RGD ligand binding (see above). Thus it may be that flexion/extension at the knees is an outcome, rather than a cause, of activation which may contribute to post-ligand binding event(s).

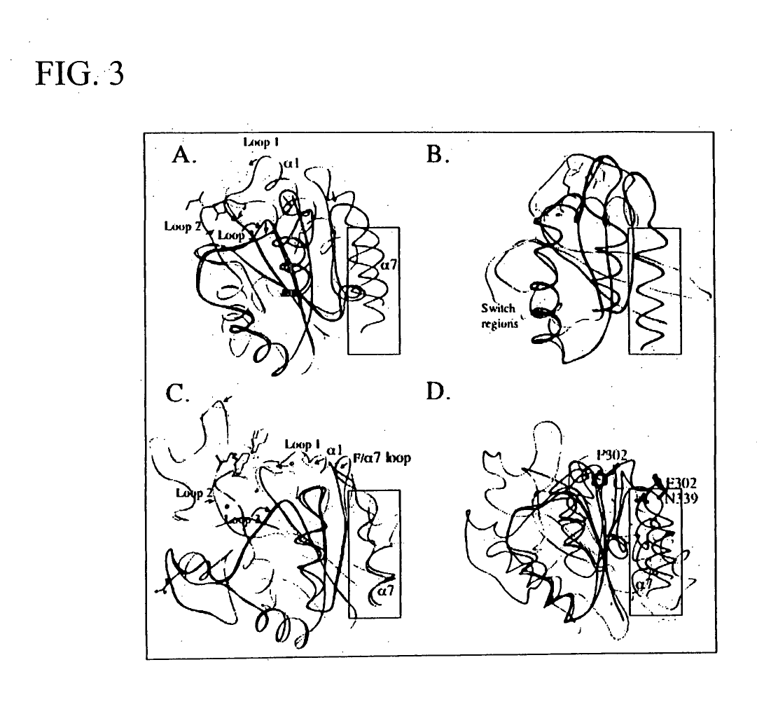

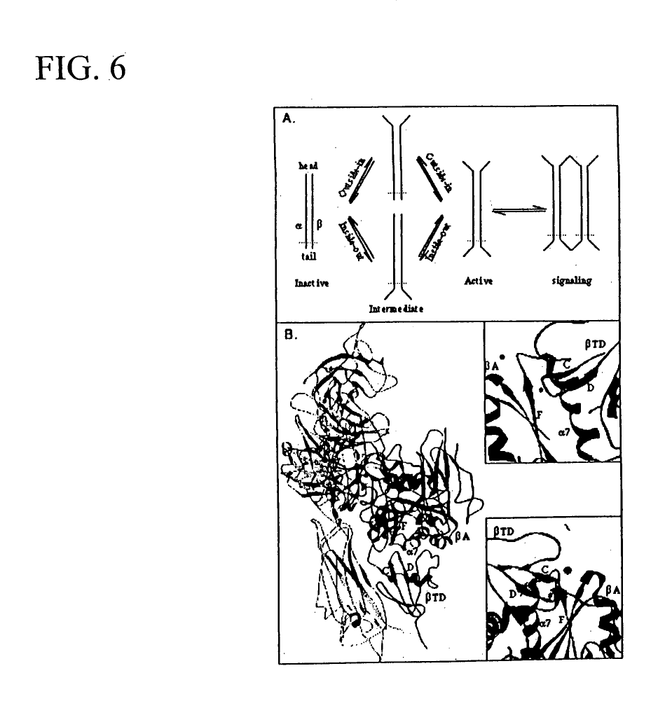

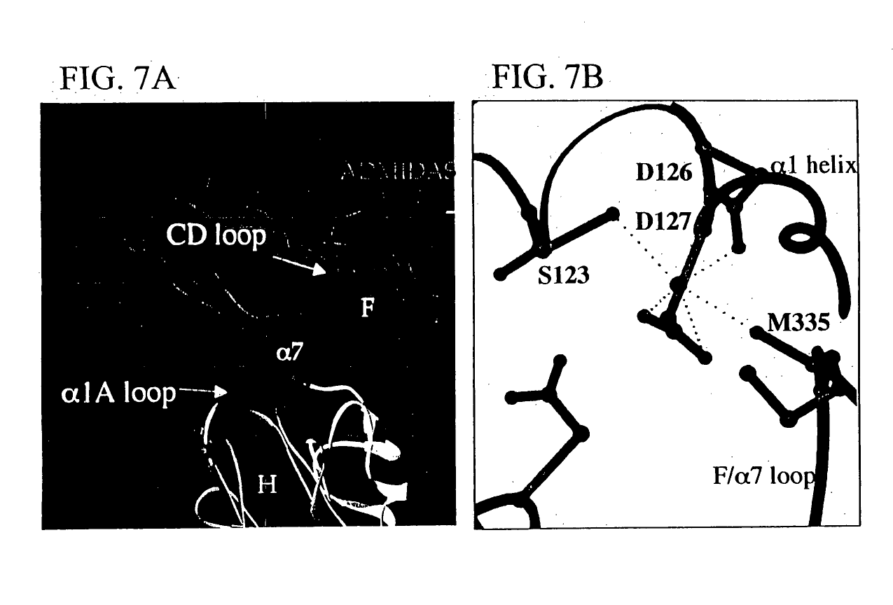

The flexible CD loop of the βTD contacts strand-F/α7 loop (Ser674 of the former contacts Val332 in βA) in unliganded αVβ3 (Table 1)(FIG. 6B), a region in βA that undergoes the most dramatic change upon RGD binding (FIG. 3C); this contact is lost in the liganded structure. The contact covers a very small surface area in unliganded αVβ3 and the βTD loop has high temperature factors. Thus this contact probably does not contribute much stabilizing energy in the crystal structure. The same side of the βTD (α1-strand-A loop) also makes a larger contact with the hybrid domain (Table 2). The close proximity of the βTD and βA domains may produce a more substantial contact following minor rearrangement of the α and β subunits and/or their domain interfaces in the membrane-bound structure. The βTD-βA contact can act as a modulatable or regulatable “deadbolt” to lock βA in an inactive state by preventing movement of the strand-F/α7 loop associated with the activation-initiated inward movement of the α1 helix. Separation of the integrin feet^{41,44} can lead through a piston-like, see-saw⁴¹ or rotational⁸³ movement of the membrane-embedded helices to leg separation. These movements can unlock the βTD “deadbolt”, freeing βA to exercise the protein movements observed in its liganded state, thus providing a pathway for rapid and reversible inside-out activation of an integrin. Based on this reasoning, one would not expect to see an extensive contact in an active integrin, because the deadbolt is not engaged in this structure. This may be precisely why the CD loop of βTD has high temperature factors: some mobility might help it to reengage the βA domain upon inactivation (sliding the deadbolt into place. In further support of this reasoning, binding of lovastatin at the strand-F/α7 interface in the αA domain from CD11a locks this integrin in the inactive state⁸⁴. It is noteworthy that the ADMIDAS (adjacent to metal-ion-dependent-adhesion-site) cation, which binds the activation-sensitive α1 helix of βA to the strand-F/α7 helix of the same domain, lies adjacent to the deadbolt site and may participate in the activation process by unlocking α1 when the strand-F/α7 loop flips. A particularly attractive feature of the deadbolt in inside-out activation is that it allows for comparatively easy transmission of changes from the membrane proximal βTD domain to the membrane-distal βA domain via a direct interface between the two domains. It suggests a dominant role of the bent conformation of αVβ3 observed in crystals and in solution and may explain the activating effects of mutations and/or activation-dependent changes in the PSI/EGF/βTD domains as well as in the adjacent calf-2 domain (reviewed in¹⁶), instances that may alter the position of the βTD relative to βA.

Significant progress has recently been made in elucidating the crystal structure of the integrin ectodomain in its unliganded and RGD-liganded states and the NMR structure of its cytoplasmic tails “feet”.^{22,23,44} The liganded structure helped define the basis of cation-dependent ligand binding specificity and helped in the development of models of the interaction of an integrin with more complex ligands. Comparison of the unliganded and liganded structures elucidated the tertiary changes leading to ligand binding and provided a minimalist view of the associated quaternary changes. This information resulted in the new “deadbolt inhibition” model for inside-out activation. Given the broad spectrum of diseases caused or exacerbated by dysregulated integrin function, this structural information provides a rich source for the rational development of specific therapeutic drugs targeting these receptors.

The αVβ3 structure has a curious analogy to the lovastatin binding site. Due to severe folding of the beta-3 chain, a membrane proximal loop of the βTD contacts the membrane distal F7 fold of the βA domain, at a position near where lovastatin contacts CD11a. On binding RGD-ligand, this contact is broken in a movement of 0.3 nm allowing movement within the βA domain. RGD-ligands can activate integrins. It is likely that this intrachain contact thus solves the problem of integrin activation for non-α-A domain integrins, explaining how an intracellular signal can communicate via 5 domains to the ligand-binding site, that is 23 nm away along the chain.

This mechanism suggests that the slot formed by the F1-F7 helices and the F7 pleat of the non-α A domains can be a general novel target, and will allow a new category of drugs to be rationally designed. These drugs will prevent integrin activation by locking the βA domain structure into a non-activatable form. The drugs would necessarily be completely different, and function by a completely different mechanism to those acting at the ligand binding site, and also, because they are mimics of an intrachain ligand, are also different from the lovastatin inhibition, that acts on extracellular activators of the integrin alpha chain. As the A domains have been successfully crystallized as independent domains, a rapid elucidation of the structure of these domains from as yet uncharacterized integrins can be made (i.e., by production of the βA domains as recombinant protein, and determination of their crystal structure), to allow rational drug design.

Starting points for rational drug design include: 1) the amino acid sequence of the βTD loops in β3 (C⁶⁶³VVRFQYYE⁶⁷¹D⁶⁷²S⁶⁷³S⁶⁷⁴G⁶⁷⁵ KSILYVVEEPEC⁶⁸⁷: SEQ ID No. 1 especially the central amino acids 671-675; and K⁶¹⁸KFDREPYMTENTCNR⁶³³YCRD: SEQ ID NO.2 especially arginine 633), 2) the homologous loops in the other relevant integrin β chains, 3) the high resolution NMR or X-ray crystallographic structures of the βA domains intact in the full-length integrin or isolated as a chain fragment containing the βA domain and variable fragments of the chain or of other appropriate fusion partners to allow rapid crystallization, and 4) structural homologues of the statin family of drugs. Screening for drug candidates in high-throughput assays can utilize the following: 1) assays based on ligand binding to integrins or recombinant ligand-binding integrin fragments (e.g., vitronectin, fibrinogen, or thrombospondin binding assays and β3A domains/βTD domain binding assays), 2) perturbation of the NMR signal, 3) perturbation at the isolated integrin receptor or βA domain-containing receptor fragment, or 4) via attachment at the cellular level.

Computer Modeling

The methods of the invention employ computer-based methods for identifying compounds having a desired structure. These computer-based methods fall into two broad classes: database methods and de novo design methods. Database methods fall in two main classes, those based on a compound (i.e., a ligand of a binding site alone) or those based on the three dimensional structure of the binding site. In the former approach, the compound of interest is compared to all compounds present in a database of chemical structures and compounds whose structure is in some way similar to the compound of interest are identified. In the latter approach, all compounds in a database are docked by appropriate computer software into the binding site, and their degree of fit is evaluated and ranked. The structures in the database are based on either experimental data, generated by NMR or x-ray crystallography, or modeled three-dimensional structures based on two-dimensional protein or DNA sequence data. In de novo design methods, models of compounds whose structure is in some way similar to the compound of interest are generated by a computer program using information derived from known structures (e.g., data generated by x-ray crystallography and/or theoretical rules). Such design methods can build a compound having a desired structure in either an atom-by-atom manner or by assembling stored small molecular fragments.

The success of both database and de novo methods in identifying compounds with activities similar to the compound of interest depends on the identification of the functionally relevant portion of the compound of interest. For drugs, the functionally relevant portion is referred to a pharmacophore. A pharmacophore then is an arrangement of structural features and functional groups important for biological activity.

Not all identified compounds having the desired pharmacophore will act as an integrin modulator. The actual activity can be finally determined only by measuring the activity of the compound in relevant biological assays. However, the methods of the invention are extremely valuable because it can be used to greatly reduce the number of compounds which must be tested to identify an actual mimetic.

Programs suitable for generating predicted three-dimensional structures from two-dimensional data include: Concord (Tripos Associated, St. Louis, Mo.), 3-D Builder (Chemical Design Ltd., Oxford, U.K.), Catalyst (Bio-CAD Corp., Mountain View, Calif.), Daylight (Abbott Laboratories, Abbott Park, Ill.).

Programs suitable for searching three-dimensional databases to identify molecules bearing a desired pharmacophore include: MACCS-3D and ISIS/3D (Molecular Design Ltd., San Leandro, Calif.), ChemDBS-3D (Chemical Design Ltd., Oxford, U.K.), Sybyl/3DB Unity (Tripos Associates, St. Louis, Mo.). Programs suitable for pharmacophore selection and design include: DISCO (Abbott Laboratories, Abbott Park, Ill.), Catalyst (Bio-CAD Corp., Mountain View, Calif.), and ChemDBS-3D (Chemical Design Ltd., Oxford, U.K.).

Databases of chemical structures are available from Cambridge Crystallographic Data Centre (Cambridge, U.K.) and Chemical Abstracts Service (Columbus, Ohio).

De novo design programs include Ludi (Biosym Technologies Inc., San Diego, Calif.) and Aladdin (Daylight Chemical Information Systems, Irvine Calif.), LEGEND (Nishibata, Y., Itai, A., Tetrahedron, 47, 8985 (1991))(Molecular Simultations, Burlington, Mass.), and LeapFrog (available from Tripos associates, St. Louis, Mo.).

Upon determination of the three-dimensional structure of an integrin, a potential modulator can be evaluated by any of several methods, alone or in combination. Such evaluation can utilize visual inspection of a three-dimensional representation of the binding site on the integrin, based on the x-ray coordinates of a crystal described herein, on a computer screen. Evaluation, or modeling, can be accomplished through the use of computer modeling techniques, hardware, and software known to those of ordinary skill in the art. This can additionally involve model building, model docking, or other analysis of protein-ligand interactions using software including, for example, QSC, GOLD (Jones et al., J. Mol. Biol., 245, 43-53, 1995), FlexX (Lengauer, Rarey, 1996), Autodock (Morris et.al., 1998), GLIDE, Modeler, or Sybyl, followed by energy minimization and molecular dynamics with standard molecular mechanics forcefields including, for example, CHARMM and AMBER. The three-dimensional structural information of an unliganded integrin, (e.g., the CD loop of the βTD contacting the strand-F/α7 loop of the βA domain in an unliganded integrin) can also be utilized in conjunction with computer modeling to generate computer models of other unliganded integrins. Computer models of unliganded integrin structures can be created using standard methods and techniques known to those of ordinary skill in the art, including software packages described herein.

Once the three-dimensional structure of a crystal, or solution structure via NMR, comprising an unliganded integrins determined, a potential non-ligand site binder (e.g., a binder that mimics the βTD binding of the strand-F/α7 loop of the βA domain) is examined through the use of computer modeling using a docking program such as QSC, GOLD, FlexX, or Autodock to identify potential non-ligand binding site binders to ascertain how well the shape and the chemical structure of the potential ligand will interact with the binding site. Computer programs can also be employed to estimate the attraction, repulsion, and steric hindrance of the two binding partners (i.e., the non-ligand binding site and the modulating binder). Generally tighter fit, lower steric hindrances, and greater attractive force between the potential ligand and the allosteric binding site are consistent with a tighter binding constant between the two. Furthermore, the more specificity in the design of a potential drug, the more likely that the drug will also not interact with other proteins. This will minimize potential side-effects due to unwanted interactions with other proteins.

A variety of methods are available to one skilled in the art for evaluating and virtually screening molecules or chemical fragments appropriate for associating with a protein, particularly an integrin. Such association can be in a variety of forms including, for example, steric interactions, van der Waals interactions, electrostatic interactions, solvation interactions, charge interactions, covalent bonding interactions, non-covalent bonding interactions (e.g., hydrogen-bonding interactions), entropically or enthalpically favorable interactions, and the like.

Numerous computer programs are available and suitable for rational drug design and the processes of computer modeling, model building, and computationally identifying, selecting and evaluating potential modulating compounds in the methods described herein. These include, for example, QSC (WO 01/98457), FlexX, Autodock, Glide, Accelrys' Discovery Studio, or Sybyl. Potential inhibitors can also be computationally designed “de novo” using such software packages as QSC (WO 01/98457), Accelrys' Discovery Studio, Sybyl, ISIS, ChemDraw, or Daylight. Compound deformation energy and electrostatic repulsion, can be evaluated using programs such as GAUSSIAN 92, AMBER, QUANTA/CHARMM, AND INSIGHT II/DISCOVER.

There are a number of ways to select moieties to fill individual binding pockets. These include QUANTA [Molecular Simulations, Inc., Burlington, Mass., 1992], SYBYL [Molecular Modeling Software, Tripos Associates, Inc., St. Louis, Mo., 1992], AMBER [S. J. Weiner, P. A. Kolhman, D. A. Case, U. C. Singh, C. Ghio, G. Alagona, and P. Weiner, J. Am. Chem. Soc., vol. 106, pp. 765-784 (1984)], or CHARMM [B. R. Brooks, R. E. Bruccoleri, B. D. Olafson, D. J. States, S Swaminathan, and M. Karplus, J. Comp. Chem. vol. 4, pp. 187-217 (1983)]. This modelling step may be followed by energy minimization with standard molecular mechanics forcefields such as CHARMM and AMBER. In addition, there are a number of more specialized computer programs to assist in the process of selecting the binding moieties of this invention.

These include:

1.) GRID (Goodford, P. J. A Computational Procedure for Determining Energetically Favorable Binding Sites on Biologically Important Macromolecules. J. Med. Chem., 28, pp. 849-857 (1985)). GRID is available from Oxford University, Oxford, UK.

2.) MCSS (Miranker, A.; Karplus, M. Functionality Maps of Binding Sites: A Multiple Copy Simultaneous Search Method. Proteins: Structure, Function and Genetics, 11, pp. 29-34 (1991)). MCSS is available from Molecular Simulations, Burlington, Mass.

3.) AUTODOCK (Goodsell, D. S.; Olsen, A. J. Automated Docking of Substrates to Proteins by Simmulated Annealing. PROTEINS: Structure, Function and Genetics, 8, pp. 195-202 (1990)). AUTODOCK is available from the Scripps Research Institute, La Jolla, Calif.

4.) DOCK (Kuntz, I. D.; Blaney, J. M.; Oatley, S. J.; Langridge, R.; Ferrin, T. E. A Geometric Approach to Macromolecule-Ligand Interactions. J. Mol. Biol., 161, pp. 269-288 (1982)). DOCK is available from the University of California, San Francisco, Calif.

5.) GOLD (Jones et al., J. Mol. Biol., 245, 43-53, 1995). GOLD is available from the Cambridge Crystallography Data Centre, Camdridge, UK.

6.) FlexX (T. Lengauer and M. Rarey, Computational Methods for Biomolecular Docking, Current Opinion in Structural Biology, Vol. 6, pp. 402-406, 1996). FlexX is available through Tripos Associated, St. Louis, Mo.

These computer evaluation and modeling techniques can be performed on any suitable hardware including for example, workstations available from Silicon Graphics, Sun Microsystems, and the like. These techniques, methods, hardware and software packages are representative and are not intended to be comprehensive listing. Other modeling techniques known in the art can also be employed in accordance with this invention. See for example, QSC (WO 01/98457), FlexX, Autodock, Glide, Accelrys' Discovery Studio, or Sybyl and software identified at various internet sites (e.g., netsci.org/Resources/Software/Modeling/CADD/

ch.cam.ac.uk/SGTL/software.html

cmm.info.nih.gov/modeling/universal_software.html

dasher.wustl.edu/tinker/

zeus.polsl.gliwice.pl/˜nikodem//linux4chemistry.html

nyu.edu/pages/mathmol/software.html

msi.umn.edu/user_support/software/MolecularModeling.html

us.expasy.org/

sisweb.com/software/model.htm).

A potential integrin modulator is selected by performing rational drug design with the three-dimensional structure (or structures) determined for the site on the βA domain, at which the βTD is in contact, described herein, in conjunction with or solely by computer modeling and methods described above. The potential modulator is then obtained from commercial sources or is synthesized from readily available starting materials using standard synthetic techniques and methodologies known to those of ordinary skill in the art. The potential inhibitor is then assayed to determine its ability to modulate the target (e.g., integrin, e.g., αVβ3) and/or integrin pathway.

A potential inhibitor can also be selected by screening a library of compounds (e.g., a combinatorial library, e.g., a mass-coded combinatorial library). The library of compounds can be screened by affinity screening in which members with the greatest affinity to a particular integrin at the new non-ligand binding site can be selected.

Once suitable binding moieties have been selected, they can be assembled into a single modulating binder. This assembly may be accomplished by connecting the various moieties to a central scaffold. The assembly process may, for example, be done by visual inspection followed by manual model building, again using software such as Quanta or Sybyl. A number of other programs may also be used to help select ways to connect the various moieties. These include: CAVEAT (Bartlett, P. A.; Shea, G. T.; Telfer, S. J.; Waterman, S. CAVEAT: A Program to Facilitate the Structure-Derived Design of Biologically Active Molecules. In “Molecular Recognition in Chemical and Biological Problems,” Special Pub., Royal Chem. Soc., 78, pp. 182-196 (1989))(available from the University of California, Berkeley, Calif.), 3D Database systems such as MACCS-3D (MDL Information Systems, San Leandro, Calif. (reviewed by Martin (Martin, Y. C. 3D Database Searching in Drug Design. J. Med. Chem., 35, pp. 2145-2154 (1992))), and HOOK (available from Molecular Simulations, Burlington, Mass.).

A number of techniques commonly used for modeling drugs may be employed (for a review, see: Cohen, N.C.; Blaney, J. M.; Humblet, C.; Gund, P.; Barry, D.C., “Molecular Modeling Software and Methods for Medicinal Chemistry”, J. Med. Chem., 33, pp. 883-894 (1990)). There are likewise a number of examples in the chemical literature of techniques that can be applied to specific drug design projects (for a review, see: Navia, M. A. and Murcko, M. A., “The Use of Structural Information in Drug Design”, Current Opinions in Structural Biology, 2, pp. 202-210 (1992)). Some examples of these specific applications include: Baldwin, J. J. et al., “Thienothiopyran-2-sulfonamides: Novel Topically Active Carbonic Anhydrase Inhibitors for the Treatment of Glaucoma”, J. Med. Chem., 32, pp. 2510-2513 (1989); Appelt, K. et al., “Design of Enzyme Inhibitors Using Iterative Protein Crystallographic Analysis”, J. Med. Chem., 34, pp. 1925-1934 (1991); and Ealick, S. E. et al., “Application of Crystallographic and Modeling Methods in the Design of Purine Nucleotide Phosphorylase Inhibitors” Proc. Nat. Acad. Sci USA, 88, pp. 11540-11544 (1991).

A variety of conventional techniques may be used to carry out each of the above evaluations as well as the evaluations necessary in screening a candidate compound in modulation (e.g., inhibition) of an integrin. Generally, these techniques involve determining the location and binding proximity of a given moiety, the occupied space of a bound modulator (e.g., inhibitor), the deformation energy of binding of a given compound and electrostatic interaction energies. Examples of conventional techniques useful in the above evaluations include: quantum mechanics, molecular mechanics, molecular dynamics, Monte Carlo sampling, systematic searches and distance geometry methods (G. R. Marshall, Ann. Ref. Pharmacol. Toxicol., 27, p. 193 (1987)). Specific computer software has been developed for use in carrying out these methods. Examples of programs designed for such uses include: Gaussian 92, revision E.2 (M. J. Frisch, Gaussian, Inc., Pittsburgh, Pa. © 1993); AMBER, version 4.0 (P. A. Kollman, University of California at San Francisco, © 1993); QUANTA/CHARMM [Molecular Simulations, Inc., Burlington, Mass. © 1992]; and Insight IL/Discover (Biosysm Technologies Inc., San Diego, Calif. © 1992). These programs may be implemented, for instance, using a Silicon Graphics Indigo 2 workstation or IBM RISC/6000 workstation model 550. Other hardware systems and software packages will be known and of evident applicability to those skilled in the art.

Conventional Screening

In addition to the computer-based technologies, the invention shows that the βTD binding interaction with the βA domain can be used in conventional drug library screening, to directly identify compounds capable of modulating the interaction between these two domains.

These assays may be based on binding and interaction assays where one partner is marked (e.g., by biotin or fluorescent labelling) and the other partner immobilized (e.g., on 96-well ELISA plates). Compound libraries are screened for their ability to enhance or block the interaction between the immobilized and the added biotinylated or fluorescent partner. Binding interaction is measured by anti-biotin antibodies, or fluorescence spectrometry analogous to the method described in detail for the αVβ3-vitronectin binding assay, below. Many other labelling technologies are usable in this method (e.g., radioactive marking, proximity assay).

Alternatively the interaction between the domains can be generated in a yeast two-hybrid system, using the βA domain (or larger protein fragment containing that domain) as bait and the βTD domain (or larger protein fragment containing that domain) as prey. Compound libraries can be tested for their ability to perturb the transcription of a suitable marker gene on a Gal4 promoter.

Compound Synthesis

Synthetic chemistry transformations and protecting group methodologies (protection and deprotection) useful in synthesizing the modulating compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., Jolm Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The modulating compounds described herein can contain one or more asymmetric centers and thus occur as racemates and racemic mixtures, single enantiomers, individual diastereomers and diastereomeric mixtures. All such isomeric forms of these compounds are expressly included in the present invention. The modulating compounds described herein can also be represented in multiple tautomeric forms, all of which are included herein. The modulating compounds can also occur in cis- or trans- or E- or Z-double bond isomeric forms. All such isomeric forms of such modulating compounds are expressly included in the present invention.

Conservative Amino Acid Substitution

Polypeptide mimetic compounds can have a different amino acid content as the polypeptide of SEQ ID No. 1 or SEQ ID No.2 and serve as a useful mimetic. Substitution mutants can include amino acid residues that represent either a conservative or non-conservative change (or, where more than one residue is varied, possibly both). A “conservative” substitution is one in which one amino acid residue is replaced with another having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The invention includes polypeptides that include one, two, three, five, or more conservative amino acid substitutions, where the resulting mutant polypeptide binds a non-ligand binding site of the integrin βA domain (e.g., strand-F/α7 loop in the βA domain of unliganded αVβ3).

Fragments or other mutant nucleic acids can be made by mutagenesis techniques well known in the art, including those applied to polynucleotides, cells, or organisms (e.g., mutations can be introduced randomly along all or part of the nucleic acid encoding the polypeptide of SEQ ID No. 1 by saturation mutagenesis), and the resultant proteins can be screened for ability to inhibit integrin activation as seen in one or more of the following assays.

Integrin Inhibition Assays

The utility of the methods of identifying compounds and compounds of the present invention can be assessed by testing in one or more of the following assays as described in detail below and further described in U.S. Pat. No. 6,489,333: Purified αVβ3 (human placenta)—Vitronectin ELISA, αVβ3—Vitronectin Binding Assay, Human Aortic Smooth Muscle Cell Migration Assay, In Vivo Angiogenesis Model, Pig Restenosis Model, Mouse Retinopathy Model. The assays are assumed to be made appropriate for the integrin of interest and the following are not limiting and merely serve as examples. A compound identified by the present invention is considered to be active if it has an IC₅₀ or Kᵢ value of less than about 10 μM for the inhibition of αVβ3-Vitronectin Binding Assay, with compounds preferably having Kᵢ values of less than about 0.1 βM. Tested compounds of the present invention are active in the αVβ3—Vitronectin Binding Assay as well as in cell-based assays of integrin adhesion mediated by the αVβ3-receptor. Generally, the assays can be adopted to more appropriately apply to the particular integrin of interest. For example, use of the appropriate ligand (e.g., RGD-containing, e.g., fibrinogen, vitronectin, fibronectin, thrombospondin, laminin, collagen, VCAM-1, ICAM-1, ICAM-2, Factor X, osteopontin, bone sialoprotein, or vWF), use of appropriate cell types, and use of appropriate conditions which is knowledge readily available to one skilled in the art. The assays that follow are useful for detecting inhibition or modulation of αVβ3, may also be directly applied to the testing of other integrins, or may be minimally modified for appropriate use for other integrins.

Purified αVβ3 (Human Placenta)—Vitronectin ELISA

The αVβ3 receptor can be isolated from human placental extracts prepared using octylglucoside. The extracts can be passed over an affinity column composed of anti-αVβ3 monoclonal antibody (LM609) to Affigel. The column can subsequently be washed extensively at pH 7 and pH 4.5 followed by elution at pH 3. The resulting sample can be concentrated by wheat germ agglutinin chromatography and can be identified by the presence of two bands on SDS gel and confirmed as αVβ3 by western blotting. The receptor can also be prepared in a soluble recombinant form using baculovirus expression as described.²¹

Affinity purified protein can be diluted at different levels and plated to 96 well plates. ELISA can be performed using fixed concentration of biotinylated vitronectin (approximately 80 nM/well). This receptor preparation is confirmed to contain the αVβ3 with no detectable levels of αVβ5 by gel (αVβ3) and by testing the effects of blocking antibodies for the αVβ3 or αVβ5 in the ELISA.

A submaximal concentration of biotinylated vitronectin can be selected based on a concentration response curve with a fixed concentration of receptor and variable concentrations of biotinylated vitronectin.

αVβ3—Vitronectin Binding Assay

Integrin-ligand binding interactions can be measured as detailed previously²¹. The purified receptor can be diluted with coating buffer (20 mM Tris HCl, 150 mM NaCl, 1.0 mM CaCl₂, 1.0 mM MgCl₂6H₂O, 10.0 μM MnCl₂.4H₂ O) and coated (100 μL/well) on Costar (3590) high capacity binding plates overnight at 4° C. The coating solution is discarded and the plates washed once with blocking/binding buffer (B/B buffer, 50 mM Tris HCl, 100 mM NaCl, 1.0 mM CaCl₂, 1.0 mM MgCl₂.6H₂O, 10.0 μM MnCl₂.4H₂ O). Receptor is then blocked (200 μL/well) with 3.5% BSA in B/B buffer for 2 hours at room temperature. After washing once with 1.0% BSA in B/B buffer, biotinylated vitronectin (100 μL) and either inhibitor (11 μL) or B/B buffer w/1.0% BSA (11 μL) is added to each well. The plates are incubated 2 hours at room temperature. The plates are washed twice with B/B buffer and incubated 1 hour at room temperature with anti-biotin alkaline phosphatase (100 μL/well) in B/B buffer containing 1.0% BSA. The plates are washed twice with B/B buffer and alkaline phosphatase substrate (100 μL) is added. Color is developed at room temperature. Color development is stopped by addition of 2N NaOH (25 μL/well) and absorbance is read at 405 nm. The IC₅₀ is the concentration of test substance needed to block 50% of the vitronectin binding to the receptor. A compound is considered to be active if it has an IC₅₀ value of less than or equal to about 10 μM in the αVβ3—Vitronectin Binding Assay. Compounds with an IC₅₀ less than 100 nM for the inhibition of vitronectin are desirable. Using the methodology described above, a number of compounds of the present invention can be found to exhibit an IC₅₀ of less than or equal to about 10 μM, thereby confirming the utility of the compounds of the present invention as effective αVβ3 integrin inhibitors.

βTD-βA Domain Binding Assay

Purified βTD as Fc-fusion protein is immobilized and the interaction with the biotinylated βA domain is measured as described above for the integrin αVβ3—vitronectin binding interaction. The IC₅₀ is the concentration of test substance needed to block 50% of the βTD binding to the receptor. A compound is considered to be active if it has an IC₅₀ value of less than or equal to about 10 μM in the βTD-βA domain binding assay. Compounds with an IC₅₀ less than 100 nM for the inhibition of βTD-βA interaction-are desirable.

Integrin Cell-Based Adhesion Assays

In the adhesion assays, a 96 well plate are coated with the appropriate ligand (e.g., fibrinogen, vitronectin, fibronectin, thrombospondin, laminin, collagen, VCAM-1, ICAM-1, ICAM-2, Factor X, osteopontin, bone sialoprotein, or vWF) for the integrin to be tested and incubated overnight at 4° C. The following day, the cells are harvested, washed, and loaded with a fluorescent dye. Compounds and cells are added together and then are immediately added to the coated plate. After incubation, loose cells are removed from the plate, and the plate (with adherent cells) is counted on a fluorometer. The ability of test compounds to inhibit cell adhesion by 50% is given by the IC₅₀ value and represents a measure of potency of inhibition of integrin mediated binding. Compounds are tested for their ability to block cell adhesion using integrin interaction assays specific for the integrin of interest.

Platelet Aggregation Assay

Venous blood is obtained from anesthetized mongrel dogs or from healthy human donors who are drug- and aspirin-free for at least two weeks prior to blood collection. Blood is collected into citrated Vacutainer tubes. The blood is centrifuged for 15 minutes at 150×g (850 RPM in a Sorvall RT6000 Tabletop Centrifuge with H-1000 B rotor) at room temperature, and platelet-rich plasma (PRP) is removed. The remaining blood is centrifuged for 15 minutes at 1500×g (26, 780 RPM) at room temperature, and platelet-poor plasma (PPP) is removed. Samples are assayed on a PAP-4 Platelet Aggregation Profiler, using PPP as the blank (100% transmittance). 200 μL of PRP (5×10⁸ platelets/mL) are added to each micro test tube, and transmittance is set to 0%. 20 μL of ADP (10 μM) is added to each tube, and the aggregation profiles are plotted (% transmittance versus time). Test agent (20 μL) is added at different concentrations prior to the addition of the platelet agonist. Results are expressed as % inhibition of agonist-induced platelet aggregation.

Human Aortic Smooth Muscle Cell Migration Assay

A method for assessing αVβ3-mediated smooth muscle cell migration and agents which inhibit αVβ3-mediated smooth muscle cell migration is described in Liaw et al., J. Clin. Invest. (1995) 95:713-724).

In Vivo Angiogenesis Model

A quantitative method for assessing angiogenesis and antiangiogenic agents is described in Passaniti et al., Laboratory Investigation (1992) 67:519-528.

Pig Restenosis Model

A method for assessing restenosis and agents which inhibit restenosis is described in Schwartz et al., J. Am. College of Cardiology (1992) 19:267-274.

Mouse Retinopathy Model

A method for assessing retinopathy and agents which inhibit retinopathy is described in Smith et al., Invest. Ophthal. & Visual Science (1994) 35: 101-111.

Contact Points of the “Deadbolt”

Table 1 below lists the contact points between the βTD and the βA domain. Table 2 below lists the more extensive contact points between the hybrid domain and the βTD. The amino acids listed in the source column of each table represent the contact points to which a modulator would be designed to bind. Modulators identified by the methods described herein would generally mimic the interactions of the peptides, c⁶⁶³ WRFQyyE⁶⁷¹ D⁶⁷²s⁶⁷³S⁶⁷⁴G⁶⁷⁵KSILYVVEEPEC⁶⁸⁷ (SEQ ID No. 1), and K⁶¹⁸KFDREPYMTENTCNRYCRD (SEQ ID NO. 2) specifically those listed in Table 1 and additionally those listed in Table 2, listed in the target column. The important amino acid contact to which an integrin modulator would bind include Ser673 as seen in Table 1. As seen in Table 2, the important amino acid contacts to which an integrin modulator would bind would include Arg633, Thr630, Glu628, and Arg636 (of the α1/strand A loop). The flexible CD loop of the βTD contacts strand-F/α7 loop (Ser674 of the former contacts Val332 in βA) in unliganded αVβ3 (Table 1)(FIG. 6B), a region in βA that undergoes the most dramatic change upon RGD binding (FIG. 3C); this contact is lost in the liganded structure. The contact covers a very small surface area in unliganded αVβ3 and the βTD loop has high temperature factors. Thus this contact probably does not contribute much stabilizing energy in the crystal structure. The same side of the βTD also makes a larger contact with the hybrid domain (Table 2). The close proximity of the βTD and βA domains may produce a more substantial contact following minor rearrangement of the α and β subunits and/or their domain interfaces in the membrane-bound structure. The βTD-βA contact can act as a modulatable or regulatable “deadbolt” to lock βA in an inactive state by preventing movement of the strand-F/α7 loop associated with the activation-initiated inward movement of the α1 helix.

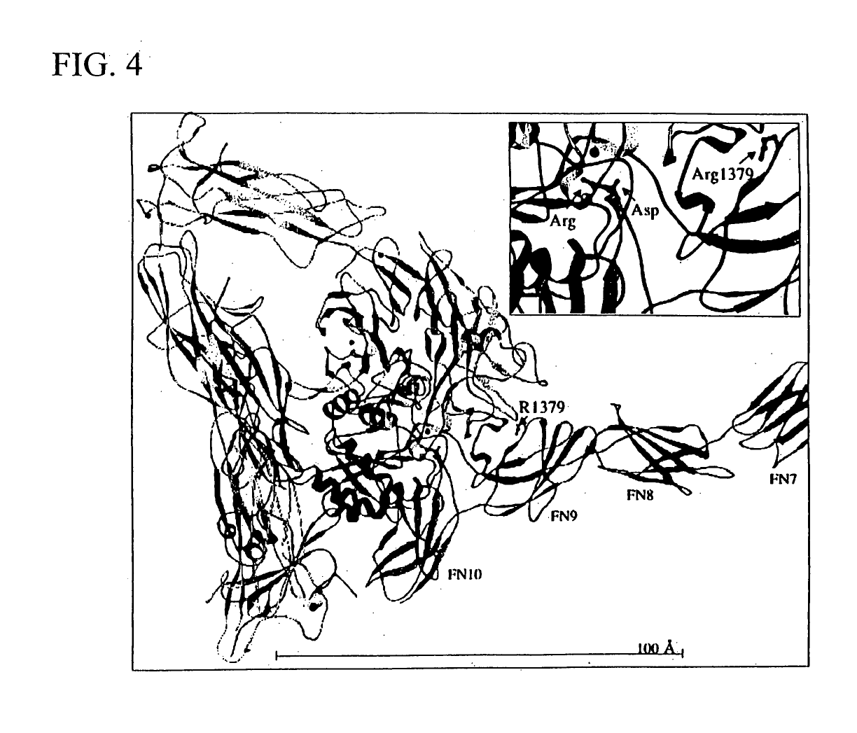

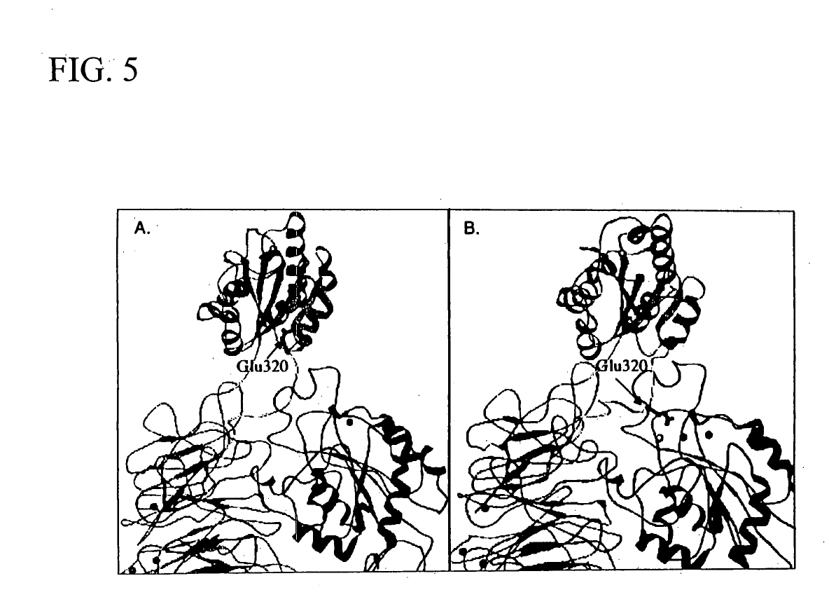

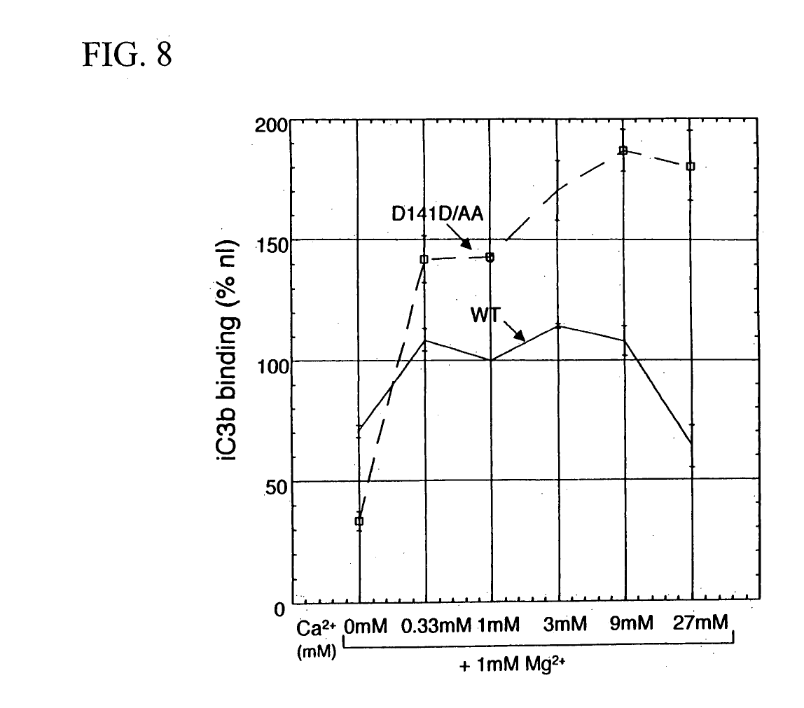

Interestingly, it was discovered that a D¹²⁶D¹²⁷/A¹²⁶A¹²⁷ mutant inactivates the ADMIDAS. Binding of the resulting membrane-bound integrin to physiologic ligand increases by 1.5-2-fold (FIG. 8). Given the location of the ADMIDAS in relation to the deadbolt, as seen in FIG. 4, it is likely that stabilizing ADMIDAS (with integrin modulators of the invention) in the unliganded state may also stabilize the deadbolt in the locked state. This region includes residues Y¹²²SMKDD (from α1) and S³³⁴NMSS (from the strand-F/α7 loop).

TABLE 2
The contact list between βA.pdb and βTD.pdb at 4.0 Å

| Source | atoms | target | | atoms | distance angle | |
|---|---|---|---|---|---|---|
| Val | 332A CG1 | ... | Ser | 673B O | ... | 3.77 |
| | | ... | Ser | 674B OG | ... | 2.80 |

TABLE 3
The contact list between hybrid.pdb and βTD.pdb at 4.0 Å

| Source | atoms | target | | atoms | distance angle | |
|---|---|---|---|---|---|---|
| Cys | 374A O | ... | Arg | 633B NH2 | ... | 3.21 *** |
| | | ... | Arg | 633B NE | ... | 3.39 * |
| | | ... | Arg | 633B CZ | ... | 3.75 |
| Leu | 375A CG | ... | Arg | 633B CB | ... | 3.90 |
| | | ... | Arg | 633B CG | ... | 3.72 |
| | | ... | Arg | 633B NE | ... | 3.63 |
| Leu | 375A CD1 | ... | Thr | 630B CG2 | ... | 3.52 |
| | | ... | Arg | 633B CB | ... | 3.46 |
| | | ... | Arg | 633B CG | ... | 3.42 |
| Leu | 375A CD2 | ... | Arg | 633B NH2 | ... | 3.80 |
| | | ... | Arg | 633B NE | ... | 3.93 |
| | | ... | Arg | 633B CZ | ... | 3.98 |
| Asn | 376A CG | ... | Glu | 628B O | ... | 3.81 |
| Asn | 376A OD | ... | Asn | 629B CB | ... | 3.71 |
| | | ... | Thr | 630B CG2 | ... | 3.52 |
| | | ... | Glu | 628B O | ... | 3.51 * |
| Asn | 376A ND2 | ... | Thr | 630B CG2 | ... | 3.71 |
| | | ... | Glu | 628B O | ... | 3.84 * |
| Met | 387A O | ... | Arg | 633B CD | ... | 3.40 |
| Gly | 388A CA | ... | Arg | 633B CD | ... | 3.94 |
| | | ... | Arg | 636B CD | ... | 3.89 |
| | | ... | Arg | 636B NH1 | ... | 3.66 |
| Gly | 388A C | ... | Arg | 633B CD | ... | 3.44 |
| | | ... | Arg | 633B NH1 | ... | 3.81 |
| Gly | 388A O | ... | Arg | 633B CA | ... | 3.78 |
| | | ... | Arg | 633B CG | ... | 3.89 |
| | | ... | Arg | 633B CD | ... | 3.03 |
| | | ... | Arg | 633B NE | ... | 3.78 * |
| | | ... | Arg | 633B CZ | ... | 3.77 |
| | | ... | Arg | 633B NH1 | ... | 2.99 *** |
| Leu | 389A CA | ... | Arg | 633B NH1 | ... | 3.53 |
| Leu | 389A CD2 | ... | Arg | 633B NH2 | ... | 3.87 |
| | | ... | Arg | 633B CD | ... | 3.61 |
| | | ... | Arg | 633B NE | ... | 3.23 |
| | | ... | Arg | 633B CZ | ... | 3.47 |
| | | ... | Arg | 633B NH1 | ... | 3.99 |
| Asp | 393A OD2 | ... | Arg | 633B NH2 | ... | 3.71 * |
| | | ... | Arg | 633B NH1 | ... | 3.41 * |

* weak bond
** strong bond
*** very strong bond

### REFERENCES

1 Hynes, R. O., Integrins: bidirectional, allosteric signaling machines. Cell 2002. 110: 673-687.
2 Humphries, M. J., Integrin structure. Biochem Soc Trans 2000. 28: 311-339.
3 Ruoslahti, E. and Pierschbacher, M. D., New perspectives in cell adhesion: RGD and integrins. Science (Wash. DC) 1987. 238: 491-497.
4 Plow, E. F., Haas, T. A., Zhang, L., Loftus, J. and Smith, J. W., Ligand binding to integrins. J Biol Chem 2000. 275: 21785-21788.
5 Dana, N., Fathallah, D. F. and Arnaout, M. A., Expression of a soluble and functional form of the human β2 integrin CD11b/CD18. Proc. Natl. Acad. Sci. (USA) 1991. 88: 3106-3110.
6 Ginsberg, M. H., Du, X. and Plow, E. F., Inside-out integrin signalling. Curr Opin Cell Biol 1992.4: 766-771.
7 Stewart, M. and Hogg, N., Regulation of leukocyte integrin function: affinity vs. avidity. J Cell Biochem 1996.61: 554-561.
8 Hato, T., Pampori, N. and Shattil, S. J., Complementary roles for receptor clustering and conformational change in the adhesive and signaling functions of integrin alphaIIb beta3. J Cell Biol 1998. 141: 1685-1695.
9 Constantin, G., Majeed, M., Giagulli, C., Piccio, L., Kim, J. Y., Butcher, E. C. and Laudanna, C., Chemokines trigger immediate beta2 integrin affinity and mobility changes: differential regulation and roles in lymphocyte arrest under flow. Immunity 2000. 13: 759-769.
10 Tsuchida, J., Ueki, S., Takada, Y., Saito, Y. and Takagi, J., The ‘ligand-induced conformational change’ of alpha 5 beta 1 integrin. Relocation of alpha 5 subunit to uncover the beta 1 stalk region. J Cell Sci 1998. 111 (Pt 12): 1759-1766.
11 Pelletier, A. J., Kunicki, T., Ruggeri, Z. M. and Quaranta, V., The activation state of the integrin alpha IIb beta 3 affects outside-in signals leading to cell spreading and focal adhesion kinase phosphorylation. J Biol Chem 1995. 270: 18133-18140.
12 Du, X., Gu, M., Weisel, J. W., Nagaswami, C., Bennett, J. S., Bowditch, R. and Ginsberg, M. H., Long range propagation of conformational changes in integrin alpha IIb beta 3. J Biol Chem 1993. 268: 23087-23092.
13 Frelinger, A. L., Du, X., Plow, E. F. and Ginsberg, M. H., Monoclonal antibodies to ligand-occupied conformers of integrin αIIbβ3 (Glycoprotein IIb-IIIa) alter receptor affinity, specificty, and function. J. Biol. Chem. 1991. 266: 17106-17111.
14 Hynes, R. O., Integrins: Versatility, modulation and signaling in cell adhesion. Cell 1992. 69: 11-26.
15 Humphries, J. D., Askari, J. A., Zhang, X. P., Takada, Y., Humphries, M. J. and Mould, A. P., Molecular basis of ligand recognition by integrin alpha5beta 1. II. Specificity of arg-gly-Asp binding is determined by Trp157 OF THE alpha subunit. J Biol Chem 2000. 275: 20337-20345.
16 Arnaout, M. A., Integrin structure: new twists and turns in dynamic cell adhesion. Immunol Rev 2002. 186: 125-140.
17 Colombatti, A., Bonaldo, P. and Doliana, R., Type A modules: Interacting domains found in several non-fibrillar collagens and in other extracellular matrix proteins. Matrix 1993. 13: 297-306.
18 Michishita, M., Videm, V. and Arnaout, M. A., A novel divalent cation-binding site in the A domain of the β2 integrin CR3 (CD11b/CD18) is essential for ligand binding. Cell 1993. 72: 857-867.
19 Lee, J.-O., Rieu, P., Arnaout, M. A. and Liddington, R., Crystal Structure of the A-domain from the α-subunit of β2 integrin complement receptor type 3 (CR3, CD11b/CD18). Cell 1995. 80: 631-638.
20 Lee, J.-O., Anne-Bankston, L., Arnaout, M. A. and Liddington, R. C., Two conformations of the integrin A-domain (I-domain): a pathway for activation? Structure 1995. 3: 1333-1340.
21 Mehta, R. J., Diefenbach, B., Brown, A., Cullen, E., Jonczyk, A., Gussow, D., Luckenbach, G. A. and Goodman, S. L., Transmembrane-truncated alphavbeta3 integrin retains high affinity for ligand binding: evidence for an ‘inside-out’ suppressor? Biochem J 1998. 330 (Pt 2): 861-869.
22 Xiong, J. P., Stehle, T., Diefenbach, B., Zhang, R., Dunker, R., Scott, D. L., Joachimiak, A., Goodman, S. L. and Arnaout, M. A., Crystal structure of the extracellular segment of integrin alpha Vbeta3. Science 2001. 294: 339-345.
23 Xiong, J. P., Stehle, T., Zhang, R., Joachimiak, A., Frech, M., Goodman, S. L. and Arnaout, M. A., Crystal structure of the extracellular segment of integrin alpha Vbeta3 in complex with an Arg-Gly-Asp ligand. Science 2002. 296: 151-155.
24 Takagi, J., Petre, B. M., Walz, T. and Springer, T. A., Global conformational rearrangements in integrin extracellular domains in outside-in and inside-out signaling. Cell 2002. 110: 599-511.
25 Bork, P., Doerks, T., Springer, T. A. and Snel, B., Domains in plexins: links to integrins and transcription factors. Trends Biochem Sci 1999. 24: 261-263.
26 Gabow, P. A. and Bennett, W. M., Renal manifestations: complication management and long-term outcome of autosomal dominant polycystic kidney disease. [Review]. Seminars in Nephrology 1991. 11: 643-652.
27 Pepinsky, R. B., Mumford, R. A., Chen, L. L., Leone, D., Amo, S. E., Riper, G. V., Whitty, A., Dolinski, B., Lobb, R. R., Dean, D.C., Chang, L. L., Raab, C. E., Si, Q., Hagmann, W. K. and Linghiam, R. B., Comparative assessment of the ligand and metal ion binding properties of integrins alpha9beta1 and alpha4beta1. Biochemistry 2002. 41: 7125-7141.
28 Feuston, B. P., Culberson, J. C., Duggan, M. E., Hartman, G. D., Leu, C. T. and Rodan, S. B., Binding Model for Nonpeptide Antagonists of alpha(v)beta(3) Integrin. J Med Chem 2002. 45: 5640-5648.
29 Emsley, J., Knight, C. G., Farudale, R. W., Barnes, M. J. and Liddington, R. C., Structural basis of collagen recognition by integrin alpha2beta1. Cell 2000. 101: 47-56.
30 Xiong, J. P., Li, R., Essafi, M., Stehle, T. and Arnaout, M. A., An isoleucine-based allosteric switch controls affinity and shape shifting in integrin CD11b A-domain. J Biol Chem 2000. 275: 38762-38767.
31 Li, R., Rieu, P., Griffith, D. L., Scott, D. and Arnaout, M. A., Two functional states of the CD11b A-domain: correlations with key features of two Mn2+-complexed crystal structures. J Cell Biol 1998. 143: 1523-1534.
32 Beglova, N., Blacklow, S. C., Takagi, J. and Springer, T. A., Cysteine-rich module structure reveals a fulcrum for integrin rearrangement upon activation. Nat Struct Biol 2002. 9: 282-287.
33 Liddington, R. C., Will the real integrin please stand up? Structure (Camb) 2002. 10: 605-607.
34 Lu, G., TOP: A new method for Protein Structure Comparisons and Similarity Searches. Journal of Appl. Cryst. 2000. 33: 176-183.
35 Jones, T., An interactive graphics program for protein crystallography. J. Appl. Crystallogr. 1978. 11: 268.
36 Woodside, D. G., Liu, S. and Ginsberg, M. H., Integrin activation. Thromb Haemost 2001. 86: 316-323.
37 O'Toole, T. E., Katagiri, Y., Faull, R. J., Peter, K., Tamura, R., Quaranta, V., Loftus, J. C., Shattil, S. J. and Ginsberg, M. H., Integrin cytoplasmic domains mediate inside-out signal transduction. J. Cell Biol. 1994. 124: 1047-1059.
38 Haas, T. A. and Plow, E. F., The cytoplasmic domain of alphaIIb beta3. A ternary complex of the integrin alpha and beta subunits and a divalent cation. J. Biol. Chem. 1996. 271: 6017-6026.
39 Vallar, L., Melchior, C., Plancon, S., Drobecq, H., Lippens, G., Regnault, V. and Kieffer, N., Divalent cations differentially regulate integrin alphaIIb cytoplasmic tail binding to beta3 and to calcium- and integrin-binding protein. J Biol Chem 1999. 274: 17257-17266.
40 Ginsberg, M. H., Yaspan, B., Forsyth, J., Ulmer, T. S., Campbell, I. D. and Slepak, M., A membrane-distal segment of the integrin alpha Ilb cytoplasmic domain regulates integrin activation. J Biol Chem 2001. 276: 22514-22521.
41 Hughes, P. E., Diaz-Gonzalez, F., Leong, L., Wu, C., McDonald, J. A., Shattil, S. J. and Ginsberg, M. H., Breaking the Integrin Hinge. J. Biol. Chem. 1996. 271: 6571-6574.
42 Li, R., Babu, C. R., Lear, J. D., Wand, A. J., Bennett, J. S. and DeGrado, W. F., Oligomerization of the integrin alphaIIbbeta3: roles of the transmembrane and cytoplasmic domains. Proc Natl Acad Sci USA 2001. 98: 12462-12467.
43 Ulmer, T. S., Yaspan, B., Ginsberg, M. H. and Campbell, I. D., NMR analysis of structure and dynamics of the cytosolic tails of integrin alpha IIb beta 3 in aqueous solution. Biochemistry 2001. 40: 7498-7508.
44 Vinogradova, O., Velyvis, A., Velyviene, A., Hu, B., Haas, T., Plow, E. and Qin, J., A structural mechanism of integrin alpha(IIb)beta(3) “inside-out” activation as regulated by its cytoplasmic face. Cell 2002. 110: 587-597.
45 Adair, B. D. and Yeager, M., Three-dimensional model of the human platelet integrin alpha IIbbeta 3 based on electron cryomicroscopy and x-ray crystallography. Proc Natl Acad Sci USA 2002. 99: 14059-14064.
46 Masumoto, A. and Hemler, M. E., Mutation of putative divalent cation sites in the a4 subunit of the integrin VLA-4: Distinct effects on adhesion to CS1/fibronectin, VCAM-1, and Invasin. J. Cell Biol. 1993. 123: 245-253.
47 Wilcox, D. A., Paddock, C. M., Lyman, S., Gill, J. C. and Newman, P. J., Glanzmann thrombasthenia resulting from a single amino acid substitution between the second and third calcium-binding domains of GPIIb. Role of the GPIIb amino terminus in integrin subunit association. J Clin Invest 1995. 95: 1553-1560.
48 Mitchell, W. B., Li, J., Singh, F., Michelson, A. D., Bussel, J., Coller, B. S. and French, D. L., Two novel mutations in the {alpha}IIb calcium-binding domains identify hydrophobic regions essential for {alpha}IIb{beta}3 biogenesis. Blood 2002.
49 Gotwals, P. J., Chi-Rosso, G., Ryan, S. T., Sizing, I., Zafari, M., Benjamin, C., Singh, J., Venyaminov, S. Y., Pepinsky, R. B. and Koteliansky, V., Divalent cations stabilize the alpha 1 beta 1 integrin I domain. Biochemistry 1999. 38: 8280-8288.
50 Masumoto, A. and Hemler, M. E., Multiple activation states of VLA-4. Mechanistic differences between adhesion to CS1/fibronectin and to vascular cell adhesion molecule-1. J Biol Chem 1993.268: 228-234.
51 Smith, J. W., Piotrowicz, R. S. and Mathis, D., A mechanism for divalent cation regulation of beta 3-integrins. Journal of Biological Chemistry 1994. 269: 960-967.
52 Mould, A. P., Getting integrins into shape: recent insights into how integrin activity is regulated by conformational changes. J Cell Sci 1996. 109 (Pt 11): 2613-2618.
53 Yan, B., Hu, D. D., Knowles, S. K. and Smith, J. W., Probing chemical and conformational differences in the resting and active conformers of platelet integrin alpha(IIb)beta(3). J Biol Chem 2000. 275: 7249-7260.
54 Chen, L. L., Whitty, A., Scott, D., Lee, W. C., Cornebise, M., Adams, S. P., Petter, R. C., Lobb, R. R. and Pepinsky, R. B., Evidence that ligand and metal ion binding to integrin alpha 4beta 1 are regulated through a coupled equilibrium. J Biol Chem 2001. 276: 36520-36529.
55 Grzesiak, J. J., Davis, G. E., Kirchhofer, D. and Pierschbacher, M. D., Regulation of alpha 2 beta 1-mediated fibroblast migration on type I collagen by shifts in the concentrations of extracellular Mg2+ and Ca2+. J Cell Biol 1992. 117: 1109-1117.
56 de Chateau, M., Chen, S., Salas, A. and Springer, T. A., Kinetic and mechanical basis of rolling through an integrin and novel Ca2+-dependent rolling and Mg2+-dependent firm adhesion modalities for the alpha 4 beta 7-MAdCAM-1 interaction. Biochemistry 2001. 40: 13972-13979.
57 Xiong, Y. M. and Zhang, L., Structure-function of the putative I-domain within the integrin beta 2 subunit. J Biol Chem 2001. 276: 19340-19349.
58 Hu, D. D., Barbas, C. F. and Smith, J. W., An allosteric Ca2+ binding site on the beta3-integrins that regulates the dissociation rate for RGD ligands. J Biol Chem 1996. 271: 21745-21751.
59 Dechantsreiter, M. A., Planker, E., Matha, B., Lohof, E., Holzemann, G., Jonczyk, A., Goodman, S. L. and Kessler, H., N-Methylated cyclic RGD peptides as highly active and selective alpha(V)beta(3) integrin antagonists. J Med Chem 1999. 42: 3033-3040.
60 Arnaout, M. A., Goodman, S. L. and Xiong, J. P., Coming to grips with integrin binding to ligands. Curr Opin Cell Biol 2002. 14: 641-651.
61 Leahy, D. J., Aukhil, I. and Erickson, H. P., 2.0 A crystal structure of a four-domain segment of human fibronectin encompassing the RGD loop and synergy region. Cell 1996. 84: 155-164.
62 Redick, S. D., Settles, D. L., Briscoe, G. and Erickson, H. P., Defining fibronectin's cell adhesion synergy site by site-directed mutagenesis. J Cell Biol 2000. 149: 521-527.
63 Kauf, A. C., Hough, S. M. and Bowditch, R. D., Recognition of fibronectin by the platelet integrin alpha IIb beta 3 involves an extended interface with multiple electrostatic interactions. Biochemistry 2001. 40: 9159-9166.
64 Copie, V., Tomita, Y., Akiyama, S. K., Aota, S., Yamada, K. M., Venable, R. M., Pastor, R. W., Krueger, S. and Torchia, D. A., Solution structure and dynamics of linked cell attachment modules of mouse fibronectin containing the RGD and synergy regions: comparison with the human fibronectin crystal structure. J Mol Biol 1998. 277: 663-682.
65 Goodman, T. G. and Bajt, M. L., Identifying the putative metal ion-dependent adhesion site in the beta2 (CD18) subunit required for alphaLbeta2 and alphaMbeta2 ligand interactions. J Biol Chem 1996. 271: 23729-23736.
66 Puzon-McLaughlin, W. and Takada, Y., Critical residues for ligand binding in an I domain-like structure of the integrin β1 subunit. J. Biol. Chem. 1996. 271: 20438-20443.
67 Alonso, J. L., Essafi, M., Xiong, J. P., Stehle, T. and Arnaout, M. A., Does the integrin alphaA domain act as a ligand for its betaA domain? Curr Biol 2002. 12: R340-342.
68 Huth, J. R., Olejniczak, E. T., Mendoza, R., Liang, H., Harris, E. A., Lupher, M. L., Jr., Wilson, A. E., Fesik, S. W. and Staunton, D. E., NMR and mutagenesis evidence for an I domain allosteric site that regulates lymphocyte function-associated antigen 1 ligand binding. Proc NatlAcad Sci USA 2000. 97: 5231-5236.
69 Lu, C., Shimaoka, M., Zang, Q., Takagi, J. and Springer, T. A., Locking in alternate conformations of the integrin alphaLbeta2 I domain with disulfide bonds reveals functional relationships among integrin domains. Proc Natl Acad Sci USA 2001. 98: 2393-2398.
70 Du, X., Plow, E. F., Frelinger, A. L., O'Toole, T. E., Loftus, J. C. and Ginsberg, M. H., Ligands “activate” integrin aIIbb3 (platelet GPIIb-IIIa). Cell 1991. 65: 409-416.
71 Legler, D. F., Wiedle, G., Ross, F. P. and Imhof, B. A., Superactivation of integrin alphavbeta3 by low antagonist concentrations. J Cell Sci 2001. 114: 1545-1553.
72 Takagi, J., Erickson, H. P. and Springer, T. A., C-terminal opening mimics ‘inside-out’ activation of integrin alpha5beta1. Nat Struct Biol 2001. 8: 412-416.
73 Mazurov, A. V., Khaspekova, S. G., Byzova, T. V., Tikhomirov, O., Berndt, M. C., Steiner, B. and Kouns, W. C., Stimulation of platelet glycoprotein IIb-IIIa (alpha IIb beta 3-integrin) functional activity by a monoclonal antibody to the N-terminal region of glycoprotein IIIa. FEBS Lett 1996. 391: 84-88.
74 Andrieux, A., Rabiet, M. J., Chapel, A., Concord, E. and Marguerie, G., A highly conserved sequence of the Arg-Gly-Asp-binding domain of the integrin beta 3 subunit is sensitive to stimulation. J Biol Chem 1991. 266: 14202-14207.
75 Kouns, W. C., Steiner, B., Kunicki, T. J., Moog, S., Jutzi, J., Jennings, L. K., Cazenave, J. P. and Lanza, F., Activation of the fibrinogen binding site on platelets isolated from a patient with the Strasbourg I variant of Glanzmann's thrombasthenia. Blood 1994. 84: 1108-1115.
76 Calvete, J. J., On the structure and function of platelet integrin alpha IIb beta 3, the fibrinogen receptor. Proc Soc Exp Biol Med 1995. 208: 346-360.
77 Sims, P. J., Ginsberg, M. H., Plow, E. F. and Shattil, S. J., Effect of platelet activation on the conformation of the plasma membrane glycoprotein IIb-IIIa complex. J Biol Chem 1991. 266: 7345-7352.
78 Valentin, N., Visentin, G. P. and Newman, P. J., Involvement of the cysteine-rich domain of glycoprotein IIa in the expression of the human platelet alloantigen, PIA1: evidence for heterogeneity in the humoral response. Blood 1995. 85: 3028-3033.
79 Mould, A. P., Askari, J. A., Barton, S., Kline, A. D., McEwan, P. A., Craig, S. E. and Humphries, M. J., Integrin activation involves a conformational change in the alpha 1 helix of the beta subunit A-domain. J Biol Chem 2002. 277: 19800-19805.
80 Honda, S., Tomiyama, Y., Pelletier, A. J., Annis, D., Honda, Y., Orchekowski, R., Ruggeri, Z. and Kunicki, T. J., Topography of ligand-induced binding sites, including a novel cation-sensitive epitope (AP5) at the amino terminus, of the human integrin beta 3 subunit. J Biol Chem 1995.270: 11947-11954.
81 Mould, A. P., Garratt, A. N., Puzon-McLaughlin, W., Takada, Y. and Humphries, M. J., Regulation of integrin function: evidence that bivalent-cation-induced conformational changes lead to the unmasking of ligand-binding sites within integrin alpha5 beta1. Biochem J 1998. 331 (Pt 3): 821-828.
82 Calzada, M. J., Alvarez, M. V. and Gonzalez-Rodriguez, J., Agonist-specific structural rearrangements of integrin alpha IIbbeta 3. Confirmation of the bent conformation in platelets at rest and after activation. J Biol Chem 2002. 277: 39899-39908.
83 Gottschalk, K. E., Adams, P. D., Brunger, A. T. and Kessler, H., Transmembrane signal transduction of the alpha(IIb)beta(3) integrin. Protein Sci 2002. 11: 1800-1812.
84 Kallen, J., Welzenbach, K., Ramage, P., Geyl, D., Kriwacki, R., Legge, G., Cottens, S., Weitz-Schmidt, G. and Hommel, U., Structural basis for LFA-1 inhibition upon lovastatin binding to the CD11a I-domain. J Mol Biol 1999. 292: 1-9.
85 CCP4, CCP4, Collaborative computational project, 4, Daresbury, UK. Acta Crystallogr D 1994.50: 760.

We claim:

1. A method for evaluating the ability of a compound to associate with a molecule or molecular complex comprising a non-ligand binding site of an integrin βA domain, the method comprising:
(a) employing computational means to perform a fitting operation between the compound and β tail domain (βTD) contact region on the strand-F/α7 loop of an unliganded integrin (e.g., αVβ3); and
(b) analyzing the results of the fitting operation to quantify said association potential.

2. The method of claim 1 wherein the compound mimics the interaction of a peptide, comprising the amino acid sequence C⁶⁶³VVRFQYYE⁶⁷¹D⁶⁷²S⁶⁷³S⁶⁷⁴G⁶⁷⁵KSILYVVEEPEC⁶⁸⁷ or a fragment thereof or K⁶¹⁸KFDREPYMTENTCNR⁶³³YCRD or a fragment thereof, with the strand-F/α7 loop of a βA domain of the integrin.

3. A method for identifying a candidate selective modulator of the activity of an integrin, the method comprising:
(a) modeling test compounds that fit spatially and preferentially into a βA domain non-ligand binding site of an integrin of interest using an atomic structural model of the integrin βA domain, wherein the atomic structural model is generated using amino acid sequence comprising C⁶⁶³VVRFQYYE⁶⁷¹D⁶⁷²S⁶⁷³S⁶⁷⁴G⁶⁷⁵ KSEYVVEEPEC⁶⁸⁷ or a fragment thereof, or K⁶¹⁸KFDREPYMTENTCNR⁶³³YCRD or fragment thereof;
(b) screening test compounds in a biological assay for integrin activation characterized by binding of a test compound to the βA domain non-ligand binding site of the integrin; and
(c) identifying a test compound that selectively modulates the activity of the integrin, and optionally,
(d) screening an identified test compound in a biological assay for its ability to prevent interaction of the βA domain and the βTD domain by binding of the identified test compound to the βA domain non-ligand binding site of the integrin, and
(e) identifying the screened test compound as a compound capable of selectively modulating the activity of an integrin from the pool of test compounds. In various embodiments: the modulating comprises inhibiting the integrin activation and the modulating comprises inhibiting ligand binding to the integrin.

4. A method of identifying a candidate inhibitor of the activity of an integrin, the method comprising:
(a) introducing into a suitable computer program information defining a non-ligand binding site of an integrin βA domain, the information comprising a conformation defined by the coordinate atoms as in Table 1 or Table 2 or both as defined in FIGS. 9 and 10, wherein the program displays the three-dimensional structure thereof;
(b) creating a three dimensional structure of a test compound in the computer program;
(c) superimposing the model of the test compound on the model of the non-ligand binding site of the integrin βA domain; and
(d) assessing whether the test compound model fits spatially into the non-ligand binding site.

5. The method of claim 4 wherein the atoms are selected from those in Table 1 and Table 2.

6. A method for identifying a candidate integrin modulating compound, the method comprising:
(a) generating a three-dimensional structure of the βTD contact region of strand-F/α7 loop of an βA domain of a non-ligand bound integrin;
(b) employing the three dimensional structure to design or select the candidate integrin modulating compound, and
(c) identifying said candidate integrin modulating compound by the data obtained by steps (a) and (b).

7. The method of claim 6 further comprising (d) providing the integrin modulating compound; and (e) determining the ability of the integrin inhibitor to bind to the integrin by contacting the modulating compound with the integrin.

8. A method for identifying a candidate integrin modulating compound, the method comprising:
(a) expressing recombinant integrin fragments containing βA domain and βTD domains; and
(b) employing the protein-protein interaction of these two domains to in screening assays to identify modulators of the βA domain and βTD domain interaction.

9. The method of claim 8 further comprising: (c) providing the integrin modulating compound; and
(d) determining the ability of the integrin modulator to bind to the integrin by measuring the interaction of the modulating compound with the integrin; and (e) using the modulating compound as a basis for drug design.

10. A method of inhibiting activation of an integrin the method comprising contacting a compound with an integrin thereby locking the βA domain structure of the integrin into a non-activatable form.

11. The method of claim 10 wherein the compound mimics an intrachain ligand in its interaction with the integrin.

12. The method of claim 10 wherein the intrachain ligand comprises the sequence of SEQ ID No:1, or a fragment thereof, or SEQ ID No. 2, or a fragment thereof.

13. The method of claim 10 wherein the intrachain ligand is a member of the statin family.

14. A method of identifying an integrin modulator comprising:
(a) selecting a potential inhibitor by performing rational drug design with the three-dimensional structural coordinates of Table 1 or Table 2 or both as define in FIGS. 9 and 10, wherein selecting is performed in conjunction with computer modeling;
(b) contacting the potential inhibitor with an integrin domain; and
(c) detecting the ability of the potential inhibitor for inhibiting the integrin.

15. The method of claim 14 wherein step (c) is performed using a ligand binding assay.

16. The method of claim 14 wherein step (c) is performed using a cellular-based assay.

17. The method of claim 16 further comprising:
(d) growing a supplemental crystal comprising a complex formed between the integrin domain and a first potential inhibitor from step (a), the supplemental crystal effectively diffracts X-rays for the atomic coordinates of the complex a resolution of greater than 4.0 Å;
(e) determining the three-dimensional structure of the supplemental crystal;
(f) selecting a second potential inhibitor by performing rational drug design with the three-dimensional structure determined for the supplemental crystal, wherein selecting is performed in conjunction with computer modeling;
(g) contacting the second potential inhibitor with the integrin domain; and
(h) detecting the ability of the second potential inhibitor for inhibiting the integrin.

18. A compound identified by the method of claim 3 with the proviso that the compound is not lovastatin.

19. A pharmaceutical compositions comprising such compounds and a pharmaceutically acceptable carrier.

20. A method for modulating, inhibiting or stimulating binding of ligands or associated proteins to integrins by modifying the interaction of integrin beta-A domain (βA) with the beta-tail domain (βTD).

21. The method of claim 20 wherein the integrin is selected from the group consisting of: αVβ1, αVβ3, αVβ5, αVβ6, αVβ8, α3β1, α4β1, α5β1, α6β1, α6β4, α7β1, α9β1, α4β7, gp3b3a, α1β1, α2β1, α10β1, α11β1, LFA-1, MAC-1, and α150β95.

22. The method of claim 20 wherein the interaction of βA, with βTD is investigated using either computational or biochemical or biophysical techniques.

23. The method of claim 20 wherein either βA, βTD or both serve as structures on which the computational or biochemical or biophysical techniques are based.

24. A method for evaluating the ability of a compound to associate with a molecule or molecular complex comprising a non-ligand binding site of an integrin βA domain, the method comprising:
(a) constructing a computer model of the binding site defined by structure coordinates wherein the root mean square deviation between the structure coordinates and the structure coordinates of the amino acids of Table 1 or Table 2 according to FIGS. 9 and 10 is not more than about 4.0 Å;
(b) selecting a compound to be evaluated by a method selected from the group consisting of (i) assembling molecular fragments into the compound, (ii) selecting a compound from a small molecule database, (iii) de novo ligand design of the compound, and (iv) modifying a known inhibitor, or a portion thereof, of a protein kinase;
(c) employing computational means to perform a fitting program operation between computer models of the compound to be evaluated and the binding site in order to provide an energy-minimized configuration of the compound in the binding site; and
(d) evaluating the results of the fitting operation to quantify the association between the said compound and the binding site, whereby evaluating the ability of the compound to associate with the binding site.

25. The method according to claim 24, wherein the binding site is further defined by the structure coordinates of one or more of SEQ ID NO: 1-4 amino acids according to FIGS. 9 and 10.

26. The method according to claim 24 wherein said molecule or molecular complex is defined by the set of structure coordinates for SEQ ID NO:1-4 amino acids according to FIGS. 9 and 10.

27. The method of claim 24 wherein the root mean square deviation is not more than 3 Å.

28. The method of claim 24 wherein the root mean square deviation is not more than 2.5 Å.

29. The method of claim 24 wherein the root mean square deviation is not more than 2 Å.

30. The method of claim 24 wherein the root mean square deviation is not more than 1.5 Å.

31. A method for identifying an activator or inhibitor of an integrin a non-ligand binding site of an integrin βA domain, the method comprising:
(a) constructing a computer model of the binding site defined by structure coordinates wherein the root mean square deviation between the structure coordinates and the structure coordinates of the amino acids of Table 1 or Table 2 according to FIGS. 9 and 10 is not more than about 4.0 Å;
(b) selecting a compound to be evaluated as a potential activator or inhibitor by a method selected from the group consisting of (i) assembling molecular fragments into the compound, (ii) selecting the compound from a small molecule database, (iii) de novo ligand design of the compound, and (iv) modifying a known inhibitor, or a portion thereof, of an integrin;
(c) employing computational means to perform a fitting program operation between computer models of the compound to be evaluated and the binding site in order to provide an energy-minimized configuration of the compound in the binding site;
(d) evaluating the results of the fitting operation to quantity the association between the compound and the binding site model, whereby evaluating the ability of the the compound to associate with the said binding pocket;
(e) providing the compound; and
(f) contacting the compound with the molecule to determine the ability of the compound to activate or inhibit the molecule.

32. The method according to claim 31, wherein the binding site is further defined by the structure coordinates of one or more of SEQ ID NO:1-4 amino acids according to FIGS. 9 and 10.

33. The method according to claim 31 wherein said molecule or molecular complex is defined by the set of structure coordinates for SEQ ID NO:1-4 amino acids according to FIGS. 9 and 10.

34. The method of claim 31 wherein the root mean square deviation is not more than 3 Å.

35. The method of claim 31 wherein the root mean square deviation is not more than 2.5 Å.

36. The method of claim 31 wherein the root mean square deviation is not more than 2 Å.

37. The method of claim 31 wherein the root mean square deviation is not more than 1.5 Å.
